(19) United States
(12) Patent Application Publication
(10) Pub. No.: US 2025/0276017 A1
(43) Pub. Date: Sep. 4, 2025

(54) Compositions and Methods for Genomic Editing

(71) Applicant: Intellia Therapeutics, Inc., Cambridge, MA (US)

(72) Inventors: Ruan Oliveira, Boston, MA (US); Sung Hee Choi, Brookline, MA (US); Biao Liu, Cambridge, MA (US); Radhika Barve, Cambridge, MA (US)

(73) Assignee: Intellia Therapeutics, Inc., Cambridge, MA (US)

(21) Appl. No.: 18/980,502

(22) Filed: Dec. 13, 2024

Related U.S. Application Data
(63) Continuation of application No. PCT/US23/68499, filed on Jun. 15, 2023.
(60) Provisional application No. 63/352,990, filed on Jun. 16, 2022.

(51) Int. Cl. A61K 35/17; C12N 5/0783; C12N 9/22; C12N 15/11

(52) U.S. Cl. CPC A61K 35/17; C12N 5/0636; C12N 9/226; C12N 15/111; C12N 2310/20; C12N 2510/00

(57) ABSTRACT

Compositions and methods for ex vivo genomic editing using Neisseria meningitidis (Nine) CRISPR/Cas9 systems are disclosed. The present disclosure provides for engineered cells comprising a genetical modification for use e.g., in adoptive cell transfer therapies.

### CROSS-REFERENCE TO RELATED APPLICATION

This application is a continuation of International Application No. PCT/US2023/068499, filed Jun. 15, 2023, which claims the benefit under 35 USC 119(e) of U.S. Provisional Application No. 63/352,990, filed Jun. 16, 2022, the content of each of which is herein incorporated by reference in its entirety.

### SEQUENCE LISTING

This application contains a sequence listing, which has been submitted electronically in XML file format and is hereby incorporated by reference in its entirety. Said XML file, created on Jun. 13, 2023, is named “01155-0055-00PCT_SL.xml” and is 20,586,099 bytes in size.

### INTRODUCTION AND SUMMARY

The present disclosure relates to genomic editing using Neisseria meningitidis CRISPR/Cas9 systems.

The ability to downregulate endogenous T-cell receptor (TCR) alpha and beta units, MHC class I, and MHC class II loci is critical for many in vivo and ex vivo utilities, e.g., when using allogeneic cells (originating from a donor) for transplantation or e.g., for creating a cell population in vitro that does not activate T cells. In particular, the transfer of allogeneic cells into a subject is of great interest to the field of cell therapy. The use of allogeneic cells has been limited due to the problem of rejection by the recipient subject's immune cells, which recognize the transplanted cells as foreign and mount an attack. To avoid the problem of immune rejection, cell-based therapies have focused on autologous approaches that use a subject's own cells as the cell source for therapy, an approach that is time-consuming and costly.

Typically, immune rejection of allogeneic cells results from a mismatching of major histocompatibility complex (MHC) molecules between the donor and recipient. Within the human population, MHC molecules exist in various forms, including e.g., numerous genetic variants of any given MHC gene, i.e., alleles, encoding different forms of MHC protein. The primary classes of MHC molecules are referred to as MHC class I and MHC class II. MHC class I molecules (e.g., HLA-A, HLA-B, and HLA-C in humans) are expressed on all nucleated cells and present antigens to activate cytotoxic T cells (CD8+ T cells or CTLs). MHC class II molecules (e.g., HLA-DP, HLA-DQ, and HLA-DR in humans) are expressed on only certain cell types (e.g., B cells, dendritic cells, and macrophages) and present antigens to activate helper T cells (CD4+ T cells or Th cells), which in turn provide signals to B cells to produce antibodies.

Slight differences, e.g., in MHC alleles between individuals can cause the T cells in a recipient to become activated. During T cell development, an individual's T cell repertoire is tolerized to one's own MHC molecules, but T cells that recognize another individual's MHC molecules may persist in circulation and are referred to as alloreactive T cells. Alloreactive T cells can become activated e.g., by the presence of another individual's cells expressing MHC molecules in the body, causing e.g., graft versus host disease and transplant rejection.

Methods and compositions for reducing the susceptibility of an allogeneic cell to rejection are of interest, including e.g., reducing the cell's expression of MHC protein to avoid recipient T cell responses. In practice, the ability to genetically modify an allogeneic cell for transplantation into a subject has been hampered by the requirement for multiple genomic edits to reduce all MHC protein expression, while at the same time, avoiding other harmful recipient immune responses. For example, while strategies to deplete MHC class I protein may reduce activation of CTLs, cells that lack MHC class I on their surface are susceptible to lysis by natural killer (NK) cells of the immune system because NK cell activation is regulated by MHC class I-specific inhibitory receptors. Therefore, safely reducing or eliminating expression of MHC class I has proven challenging. Genomic editing strategies to deplete MHC class II molecules have also proven difficult particularly in certain cell types for reasons including low editing efficiencies and low cell survival rates, preventing practical application as a cell therapy.

Thus, there exists a need for improved methods and compositions for modifying cells to overcome the problem of recipient immune rejection and the technical difficulties associated with the multiple genetic modifications required to produce a safer cell for transplant. The present disclosure provides genomic editing using Neisseria meningitidis CRISPR/Cas9 systems. NmeCas9 is smaller than Streptococcus pyogenes Cas9 (SpyCas9), allowing NmeCas9 to be suitable for messenger RNA (mRNA)-based delivery methods. NmeCas9 has an advantageous specificity and low off-target cleavage rates.

The engineered cell comprises a genetic modification in the HLA-A, TRAC, TRBC, or CIITA (class II major histocompatibility complex transactivator), which may be useful in cell therapy. The disclosure further provides compositions and methods to reduce or eliminate surface expression of endogenous T-cell receptor, MHC class I or II protein in a cell by genetically modifying the HLA-A, TRAC, TRBC, or CIITA gene.

In some embodiments, a method of reducing surface expression of HLA-A protein in an engineered cell relative to an unmodified cell, is provided, the method comprising contacting a cell with a composition of any of embodiments provided herein. In some embodiments, a method of reducing surface expression of TRAC protein in an engineered cell relative to an unmodified cell, is provided, the method comprising contacting a cell with a composition of any of embodiments provided herein. In some embodiments, a method of reducing surface expression of TRBC protein in an engineered cell relative to an unmodified cell, is provided, the method comprising contacting a cell with a composition of any of embodiments provided herein. In some embodiments, a method of reducing surface expression of MHC class II protein in an engineered cell relative to an unmodified cell, is provided, the method comprising contacting a cell with a composition of any of embodiments provided herein.

### BRIEF DESCRIPTION OF THE DRAWINGS

FIG. 1A shows the distribution of percent of HLA-A2−, HLA-A3− T cells following editing with HLA-A targeting guides.

FIG. 1B shows the percent of HLA-A2− HLA-A3+ (HLA-A-), HLA-A+, HLA-A3+ (HLA-A3−) and HLA-A2−, HLA-A3− T cells following editing with select guides.

FIG. 1C shows percent HLA-B− T cells following editing with select HLA-A guides.

FIG. 2 shows percent HLA-A2 negative T cells following editing with a dilution series of HLA-A guides.

FIG. 3A shows the distribution of percent of CD3− T cells following editing with TRAC guides.

FIG. 3B shows percent editing at the TRAC locus.

FIG. 4 shows percent CD3 negative T cells following editing with a dilution series of TRAC guides.

FIG. 5A shows the distribution of percent of CD3− T cells following editing with TRBC guides.

FIG. 5B shows percent editing at the TRBC loci.

FIG. 6 shows percent CD3 negative T cells following editing with a dilution series of TRBC guides.

FIG. 7A shows the distribution of percent of HLA-DP, DQ, DR− T cells following editing with CIITA guides.

FIG. 7B shows percent editing at the CIITA locus.

FIG. 8 shows percentage of HLA II-DR, DP, DQ negative cells on the left vertical axis and mean percent editing on the right vertical axis following editing with CIITA guides.

FIG. 9A shows the percent of reads with C to T conversions at the CIITA locus following editing with a dilution series of CIITA guides.

FIG. 9B shows the percent of HLA-DP, DQ, DR negative T cells following editing with a dilution series of CIITA guides.

FIGS. 10A-10B show editing at the AAVS1 locus represented as indel frequency.

FIGS. 11A-11B show indel frequency at AAVS1 following editing with a dilution series of AAVS1 guides.

FIG. 12 shows mean editing frequency at various TRAC sgRNA concentrations.

FIG. 13 shows mean editing frequency at the CIITA locus.

FIG. 14 shows mean percentage of T cells negative for HLA-A2 surface expression.

FIG. 15 shows mean percentage editing at the CIITA locus.

FIGS. 16A-16L show mean percentage editing at a series of doses of the noted guide RNAs.

FIGS. 17A-17D show mean percentage editing of CD8+ T cells that are negative for respective expression markers at various sgRNA concentrations.

### DETAILED DESCRIPTION

The present disclosure provides engineered cells, as well as methods and compositions for genetically modifying a cell to make an engineered cell and populations of engineered cells, that are useful, for example, for adoptive cell transfer (ACT) therapies. The disclosure provided herein overcomes certain hurdles of prior methods by providing methods and compositions for genetically modifying the HLA-A, TRAC, TRBC, CIITA, or AAVS1 locus to reduce expression of HLA-A, TRAC, TRBC, or MHC class II protein on the surface of a cell. In some embodiments, the disclosure provides engineered cells with reduced or eliminated surface expression of HLA-A, TRAC, TRBC, or MHC class II as a result of a genetic modification in the HLA-A, TRAC, TRBC, or CIITA gene. In some embodiments, the disclosure provides compositions and methods for reducing or eliminating expression of HLA-A, TRAC, TRBC, or MHC class II protein and compositions and methods to further reduce the cell's susceptibility to immune rejection. For example, in some embodiments, the methods and compositions comprise reducing or eliminating surface expression of HLA-A protein by genetically modifying the HLA-A gene. In some embodiments, the methods and compositions comprise reducing or eliminating surface expression of TRAC protein by genetically modifying the TRAC gene. In some embodiments, the methods and compositions comprise reducing or eliminating surface expression of TRBC1 protein by genetically modifying the TRBC1 gene. In some embodiments, the methods and compositions comprise reducing or eliminating surface expression of TRBC2 protein by genetically modifying the TRBC2 gene. In some embodiments, the methods and compositions comprise reducing or eliminating surface expression of MHC class II protein by genetically modifying CIITA. The engineered cell compositions produced by the methods disclosed herein have desirable properties, including e.g., reduced expression of MHC molecules, reduced immunogenicity in vitro and in vivo, increased survival, and increased genetic compatibility with greater subjects for transplant.

The term “about” or “approximately” means an acceptable error for a particular value as determined by one of ordinary skill in the art, which depends in part on how the value is measured or determined, or a degree of variation that does not substantially affect the properties of the described subject matter, or within the tolerances accepted in the art, e.g., within 10%, 5%, 2%, or 1%. Accordingly, unless indicated to the contrary, the numerical parameters set forth in the following specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

Provided herein are the following numbered embodiments:
Embodiment 1 is an engineered cell, which has reduced or eliminated surface expression of HLA-A relative to an unmodified cell, comprising a genetic modification in the HLA-A gene, wherein the genetic modification comprises at least one nucleotide within the genomic coordinates chr6:29942540-29945459.
Embodiment 2 is the engineered cell of embodiment 1, which has reduced or eliminated surface expression of HLA-A relative to an unmodified cell, comprising a genetic modification in the HLA-A gene, wherein the genetic modification comprises at least one nucleotide within the genomic coordinates chosen from any one of the genomic coordinates listed in Table 1 or wherein the genetic modification comprises at least one nucleotide within the genomic coordinates targeted by a guide RNA comprising a guide sequence of any one of SEQ ID NOs: 66, 61, 2-60, 62-65, 67-80.
Embodiment 3 is an engineered cell, which has reduced or eliminated surface expression of HLA-A relative to an unmodified cell, comprising a genetic modification in the HLA-A gene, wherein the genetic modification comprises at least one nucleotide within the genomic coordinates chosen from: chr6:29942891-29942915; chr6:29942609-29942633; chr6:29944266-29944290; chr6:29942889-29942913; chr6:29944471-29944495; and chr6:29944470-29944494.
Embodiment 4 is an engineered human cell, which has reduced or eliminated surface expression of HLA-A relative to an unmodified cell, comprising a genetic modification in the HLA-A gene, wherein the genetic modification comprises at least one nucleotide within the genomic coordinates chosen from: chr6:29942891-29942915; chr6:29942609-29942633; chr6:29942889-29942913; chr6:29944471-29944495; chr6:29944266-29944290; chr6:29942785-29942809.
Embodiment 5 is an engineered human cell, which has reduced or eliminated surface expression of HLA-A relative to an unmodified cell, comprising a genetic modification in an HLA-A gene, wherein the genetic modification comprises an indel, a C to T substitution, or an A to G substitution within the genomic coordinates chosen from: chr6:29942891-29942915; chr6:29942609-29942633; chr6:29944266-29944290; chr6:29942889-29942913; chr6:29944471-29944495; and chr6:29944470-29944494.
Embodiment 6 is an engineered human cell, which has reduced or eliminated surface expression of HLA-A relative to an unmodified cell, comprising a genetic modification in an HLA-A gene, wherein the genetic modification comprises an indel, a C to T substitution, or an A to G substitution within the genomic coordinates chosen from: chr6:29942891-29942915; chr6:29942609-29942633; chr6:29942889-29942913; chr6:29944471-29944495; chr6:29944266-29944290; chr6:29942785-29942809.
Embodiment 7 is the engineered cell of any one of embodiments 1-6, wherein the HLA-A expression is reduced or eliminated by a genomic editing system that binds to an HLA-A genomic target sequence comprising at least 5 contiguous nucleotides within the genomic coordinates chosen from: chr6:29942891-29942915; chr6:29942609-29942633; chr6:29944266-29944290; chr6:29942889-29942913; chr6:29944471-29944495; and chr6:29944470-29944494 or chosen from chr6:29942891-29942915; chr6:29942609-29942633; chr6:29942889-29942913; chr6:29944471-29944495; chr6:29944266-29944290; chr6:29942785-29942809.
Embodiment 8 is the engineered cell of any one of embodiments 1-7, wherein the genetic modification comprises at least one nucleotide within the genomic coordinates chosen from any one of the genomic coordinates listed in Table 1.
Embodiment 9 is the engineered cell of any one of embodiments 1-8, wherein the cell is homozygous for HLA-C.
Embodiment 10 is the engineered cell of any one of embodiments 1-9, wherein the cell is homozygous for HLA-B and homozygous for HLA-C.
Embodiment 11 is a composition comprising an HLA-A guide RNA and optionally an RNA-guided DNA binding agent or nucleic acid encoding an RNA-guided DNA binding agent, wherein the HLA-A guide RNA comprises: i. a guide sequence selected from SEQ ID NOs: 66, 61, 2-60, 62-65, 67-80; ii. at least 19, 20, 21, 22, 23, or 24 contiguous nucleotides of a sequence selected from SEQ ID NOs: 66, 61, 2-60, 62-65, 67-80; or iii. a guide sequence that is at least 95%, 90%, or 85% identical to a sequence selected from SEQ ID NOs: 66, 61, 2-60, 62-65, 67-80; iv. a sequence that comprises 10 contiguous nucleotides±10 nucleotides of a genomic coordinate listed in Table 1; v. at least 20, 21, 22, 23, or 24, contiguous nucleotides of a sequence from (iv); or vi. a guide sequence that is at least 95%, 90%, or 85% identical to a sequence selected from (iv).
Embodiment 12 is a method of making an engineered human cell, which has reduced or eliminated surface expression of HLA-A protein relative to an unmodified cell, comprising contacting a cell with a composition comprising an HLA-A guide RNA and optionally an RNA-guided DNA binding agent or nucleic acid encoding an RNA-guided DNA binding agent, wherein the HLA-A guide RNA comprises: i. a guide sequence selected from SEQ ID NOs: 66, 61, 2-60, 62-65, 67-80; ii. at least 20, 21, 22, 23, or 24 contiguous nucleotides of a sequence selected from SEQ ID NOs: 66, 61, 2-60, 62-65, 67-80; or iii. a guide sequence that is at least 95%, 90%, or 85% identical to a sequence selected from SEQ ID NOs: 66, 61, 2-60, 62-65, 67-80; iv. a sequence that comprises 10 contiguous nucleotides±10 nucleotides of a genomic coordinate listed in Table 1; v. at least 20, 21, 22, 23, or 24 contiguous nucleotides of a sequence from (iv); or vi. a guide sequence that is at least 95%, 90%, or 85% identical to a sequence selected from (iv).
Embodiment 13 is a method of reducing surface expression of HLA-A protein in a human cell relative to an unmodified cell, comprising contacting a cell with a composition comprising an HLA-A guide RNA and optionally an RNA-guided DNA binding agent or nucleic acid encoding an RNA-guided DNA binding agent, wherein the HLA-A guide RNA comprises: i. a guide sequence selected from SEQ ID NOs: 66, 61, 2-60, 62-65, 67-80; ii. at least 20, 21, 22, 23, or 24 contiguous nucleotides of a sequence selected from SEQ ID NOs: 66, 61, 2-60, 62-65, 67-80; or iii. a guide sequence that is at least 95%, 90%, or 85% identical to a sequence selected from SEQ ID NOs: 66, 61, 2-60, 62-65, 67-80; iv. a sequence that comprises 10 contiguous nucleotides±10 nucleotides of a genomic coordinate listed in Table 1; v. at least 20, 21, 22, 23, or 24 contiguous nucleotides of a sequence from (iv); or vi. a guide sequence that is at least 95%, 90%, or 85% identical to a sequence selected from (iv).
Embodiment 14 is the composition or method of any one of embodiments 11-13, wherein the HLA-A guide RNA comprises a guide sequence of any one of SEQ ID NO: 66, 61, 13, 55, 70, and 71.
Embodiment 15 is the composition or method of any one of embodiments 11-13, wherein the HLA-A guide RNA comprises a guide sequence of any one of SEQ ID NOs: 61, 66, 13, 17, 55, and 70.
Embodiment 16 is the composition or method of any one of embodiments 11-13, wherein the HLA-A guide RNA comprises a guide sequence comprising a sequence of SEQ ID NO: 61.
Embodiment 17 is the composition or method of any one of embodiments 11-13, wherein the HLA-A guide RNA comprises a guide sequence comprising a sequence of SEQ ID NO: 66.
Embodiment 18 is a population of cells comprising the engineered cells of any one of embodiments 1-10, or the engineered cells produced by the method of any one of embodiments 12-17 or by use of the composition of embodiment 11.
Embodiment 19 is a pharmaceutical composition comprising (a) the engineered cells of any one of embodiments 1-10; the engineered cells produced by the method of any one of embodiments 12-17 or by use of the composition of embodiment 11; or (b) a population of cells of embodiment 18.
Embodiment 20 is an engineered human cell, which has reduced or eliminated surface expression of TRAC relative to an unmodified cell, comprising a genetic modification in the TRAC gene, wherein the genetic modification comprises at least one nucleotide within the genomic coordinates chr14:22547505-22551621 or chr14:22547462-22551621.
Embodiment 21 is the engineered cell of embodiment 20, wherein the genetic modification comprises at least one nucleotide within the genomic coordinates chosen from any one of the genomic coordinates listed in Table 2 or wherein the genetic modification comprises at least one nucleotide within the genomic coordinates targeted by a guide RNA comprising a guide sequence of any one of SEQ ID NOs: 111, 107, 101-106, 108-110, and 112-120.
Embodiment 22 is the engineered cell of embodiment 20 or 21, wherein the genetic modification comprises at least one nucleotide within the genomic coordinates chosen from: chr14:22550574-22550598; chr14:22550544-22550568; chr14:22547505-22547529; or chr14:22547525-22547549; chr14:22547674-22547698.
Embodiment 23 is the engineered cell of embodiment 20 or 21, wherein the genetic modification comprises at least one nucleotide within the genomic coordinates chr14:22547481-22547505.
Embodiment 24 is the engineered cell of embodiment 20 or 21, wherein the genetic modification comprises at least one nucleotide within the genomic coordinates chr14:22547471-22547495.
Embodiment 25 is the engineered cell of embodiment 20 or 21, wherein the genetic modification comprises at least one nucleotide within the genomic coordinates chr14:22547470-22547494.
Embodiment 26 is the engineered cell of embodiment 20 or 21, wherein the genetic modification comprises at least one nucleotide within the genomic coordinates chr14:22547462-22547486.
Embodiment 27 is an engineered human cell, which has reduced or eliminated expression of TRAC relative to an unmodified cell, comprising a genetic modification in the TRAC gene, wherein the genetic modification comprises an indel, a C to T substitution, or an A to G substitution within the genomic coordinates chosen from: chr14:22550574-22550598; chr14:22550544-22550568; chr14:22547505-22547529; chr14:22547525-22547549; or chr14:22547674-22547698.
Embodiment 28 is the engineered cell of any one of embodiments 20-27, wherein the TRAC expression is reduced or eliminated by a genomic editing system that binds to a TRAC target sequence comprising at least 5 contiguous nucleotides within the genomic coordinates chosen from: chr14:22550574-22550598; chr14:22550544-22550568; chr14:22547505-22547529; chr14:22547525-22547549; or chr14:22547674-22547698.
Embodiment 29 is a composition comprising: a) a TRAC guide RNA comprising a guide sequence that i) targets a TRAC genomic target sequence; or ii) directs an RNA-guided DNA binding agent to induce a double stranded break (DSB) or a single-stranded break (SSB) in a TRAC genomic target sequence comprising at least 10 contiguous nucleotides within the genomic coordinates chosen from: chr14:22550574-22550598; chr14:22550544-22550568; chr14:22547505-22547529; chr14:22547525-22547549; or chr14:22547674-22547698.
Embodiment 30 is a composition comprising: (a) a TRAC guide RNA and optionally (b) an RNA-guided DNA binding agent or a nucleic acid encoding an RNA-guided DNA binding agent, wherein the TRAC guide RNA comprises: i) a guide sequence selected from SEQ ID NOs: 111, 107, 101-106, 108-110, and 112-120; ii) at least 20, 21, 22, 23, or 24 contiguous nucleotides of a sequence selected from SEQ ID NOs: 111, 107, 101-106, 108-110, and 112-120; or iii) a guide sequence that is at least 95%, 90%, or 85% identical to a sequence selected from SEQ ID NOs: 111, 107, 101-106, 108-110, and 112-120; or iv) a sequence that comprises 10 contiguous nucleotides±10 nucleotides of a genomic coordinate listed in Table 2; v) at least 20, 21, 22, 23, or 24 contiguous nucleotides of a sequence from (iv); or vi) a guide sequence that is at least 95%, 90%, or 85% identical to a sequence selected from (iv).
Embodiment 31 is the composition of embodiment 30, for use in altering a DNA sequence within the TRAC locus in a cell.
Embodiment 32 is the composition of embodiment 30, for use in reducing or eliminating the expression of TRAC protein in a cell.
Embodiment 33 is a method of making an engineered human cell, which has reduced or eliminated surface expression of TRAC protein relative to an unmodified cell, comprising contacting a cell with a composition comprising: (a) a TRAC guide RNA and optionally (b) an RNA-guided DNA binding agent or a nucleic acid encoding an RNA-guided DNA binding agent, wherein the TRAC guide RNA comprises: i) a guide sequence selected from SEQ ID NOs: 111, 107, 101-106, 108-110, and 112-120; ii) at least 20, 21, 22, 23, or 24 contiguous nucleotides of a sequence selected from SEQ ID NOs: 111, 107, 101-106, 108-110, and 112-120; or iii) a guide sequence that is at least 95%, 90%, or 85% identical to a sequence selected from SEQ ID NOs: 111, 107, 101-106, 108-110, and 112-120; or iv) a sequence that comprises 10 contiguous nucleotides±10 nucleotides of a genomic coordinate listed in Table 2; v) at least 20, 21, 22, 23, or 24 contiguous nucleotides of a sequence from (iv); or vi) a guide sequence that is at least 95%, 90%, or 85% identical to a sequence selected from (iv).
Embodiment 34 is a method of reducing surface expression of TRAC protein in a human cell relative to an unmodified cell, comprising contacting a cell with a composition comprising: (a) a TRAC guide RNA and optionally (b) an RNA-guided DNA binding agent or a nucleic acid encoding an RNA-guided DNA binding agent, wherein the TRAC guide RNA comprises: i) a guide sequence selected from SEQ ID NOs: 111, 107, 101-106, 108-110, and 112-120; ii) at least 20, 21, 22, 23, 24, or 25 contiguous nucleotides of a sequence selected from SEQ ID NOs: 111, 107, 101-106, 108-110, and 112-120; or iii) a guide sequence that is at least 95%, 90%, or 85% identical to a sequence selected from SEQ ID NOs: 111, 107, 101-106, 108-110, and 112-120; or iv) a sequence that comprises 10 contiguous nucleotides±10 nucleotides of a genomic coordinate listed in Table 2; v) at least 20, 21, 22, 23, 24, or 25 contiguous nucleotides of a sequence from (iv); or vi) a guide sequence that is at least 95%, 90%, or 85% identical to a sequence selected from (iv).
Embodiment 35 is the composition or method of any one of embodiments 29-34, wherein the TRAC guide RNA comprises a guide sequence of any one of SEQ ID NO: 111, 107, 101, 102, and 103.
Embodiment 36 is the composition or method of any one of embodiments 29-34, wherein the TRAC guide RNA comprises a guide sequence comprising a sequence of SEQ ID NO: 107.
Embodiment 37 is the composition or method of any one of embodiments 29-34, wherein the TRAC guide RNA comprises a guide sequence comprising a sequence of SEQ ID NO: 111.
Embodiment 38 is the composition or method of any one of embodiments 29-34, wherein the TRAC guide RNA comprises a guide sequence comprising a sequence of SEQ ID NO: 117.
Embodiment 39 is the composition or method of any one of embodiments 29-34, wherein the TRAC guide RNA comprises a guide sequence comprising a sequence of SEQ ID NO: 118.
Embodiment 40 is the composition or method of any one of embodiments 29-34, wherein the TRAC guide RNA comprises a guide sequence comprising a sequence of SEQ ID NO: 119.
Embodiment 41 is the composition or method of any one of embodiments 29-34, wherein the TRAC guide RNA comprises a guide sequence comprising a sequence of SEQ ID NO: 120.
Embodiment 42 is a population of cells comprising the engineered cells of any one of embodiments 20-28 or the engineered cells produced by use of the composition of claim 29 or 30 or the method of any one of embodiments 33-41, wherein greater than about 50%, greater than about 55%, greater than about 60%, greater than about 65%, greater than about 70%, greater than about 75%, greater than about 80%, greater than about 85%, greater than about 90%, greater than about 95%, greater than about 98%, or greater than about 99% of the population of cells are CD3-cells.
Embodiment 43 is a population of cells comprising the engineered cells of any one of embodiments 20-28, or the engineered cells produced by use of the composition of embodiment 29 or 30 or by the method of any one of embodiments 33-41, or the population of cells of embodiment 42, wherein greater than about 50%, greater than about 55%, greater than about 60%, greater than about 65%, greater than about 70%, greater than about 75%, greater than about 80%, greater than about 85%, greater than about 90%, greater than about 95%, greater than about 98%, or greater than about 99% of the population lacks an endogenous T-cell receptor.
Embodiment 44 is a population of cells comprising the engineered cells of any one of embodiments 20-28, or the engineered cells produced by use of the composition of embodiment 29 or 30 or by the method of any one of embodiments 33-41, or the population of cells of embodiment 42 or 43, wherein the expression of the TRAC gene in the population has been reduced relative to an unaltered population of the same cell by at least about 50%, at least about 55%, by at least about 60%, at least about 65%, at least about 70%, by at least about 75%, at least about 80%, at least about 85%, by at least about 90%, at least about 95%, or at least about 98%, or at least about 99%.
Embodiment 45 is a pharmaceutical composition comprising the engineered cells of any one of embodiments 20-28, or the engineered cells produced by use of the composition of embodiment 29 or 30 or by the method of any one of embodiments 33-41, or the population of cells of any one of embodiments 42-44.
Embodiment 46 is an engineered cell, which has reduced or eliminated surface expression of TRBC relative to an unmodified cell, comprising a genetic modification in the TRBC locus, wherein the genetic modification comprises at least one nucleotide within the genomic coordinates chr7: 142791756-142802543.
Embodiment 47 is an engineered cell, which has reduced or eliminated surface expression of TRBC relative to an unmodified cell, comprising a genetic modification in the TRBC locus, wherein the genetic modification comprises at least one nucleotide within the genomic coordinates: (a) chr7:142791862-142793149; (b) chr7: 142791756-142792721; or (c) chr7:142801104-142802543; or wherein the genetic modification comprises at least one nucleotide within the genomic coordinates targeted by a guide RNA comprising a guide sequence of any one of SEQ ID NOs: 215, 201-214, and 216-265.
Embodiment 48 is the engineered cell of embodiments 46 or 47, wherein the genetic modification comprises at least one nucleotide within the genomic coordinates chosen from any one of the genomic coordinates listed in Table 3.
Embodiment 49 is the engineered cell of embodiments 46 or 47, wherein the genetic modification comprises at least one nucleotide within the genomic coordinates chosen from: (a) chr7:142792690-142792714; or chr7:142792693-142792717; or (b) chr7:142791756-142791780; chr7:142791761-142791785; chr7:142791820-142791844; chr7:142791939-142791963; chr7:142791940-142791964; or chr7:142792004-142792028; or (c) chr7:142801104-142801124; chr7:142802103-142802127; or chr7:142802106-142802130.
Embodiment 50 is the engineered cell of embodiment 46 or 47, wherein the genetic modification comprises at least one nucleotide within the genomic coordinates chosen from: chr7:142792690-142792714; chr7:142802103-142802127; and chr7:142802106-14280213.
Embodiment 51 is an engineered cell, which has reduced or eliminated expression of TRBC relative to an unmodified cell, comprising a genetic modification in the human TRBC gene, wherein the genetic modification comprises an indel, a C to T substitution, or an A to G substitution within the genomic coordinates (a) chr7:142791862-142793149; (b) chr7: 142791756-142792721; or (c) chr7:142801104-142802543.
Embodiment 52 is an engineered human cell, which has reduced or eliminated expression of TRBC relative to an unmodified cell, comprising a genetic modification in the TRBC gene, wherein the genetic modification comprises an indel, a C to T substitution, or an A to G substitution within the genomic coordinates chosen from: (a) chr7:142792690-142792714 or chr7:142792693-142792717; or (b) chr7:142791756-142791780; chr7:142791761-142791785; chr7:142791820-142791844; chr7:142791939-142791963; chr7:142791940-142791964; or chr7:142792004-142792028; or (c) chr7:142801104-142801124; chr7:142802103-142802127; or chr7:142802106-142802130.
Embodiment 53 is an engineered human cell, which has reduced or eliminated expression of TRBC relative to an unmodified cell, comprising a genetic modification in the TRBC gene, wherein the genetic modification comprises an indel, a C to T substitution, or an A to G substitution within the genomic coordinates chosen from: chr7:142792690-142792714; chr7:142802103-142802127; and chr7:142802106-14280213.
Embodiment 54 is the engineered cell of any one of embodiments 46-53, wherein the TRBC expression is reduced or eliminated by a genomic editing system that binds to a TRBC target sequence comprising at least 5 contiguous nucleotides within the genomic coordinates chosen from: (a) chr7:142792690-142792714 or chr7:142792693-142792717; or (b) chr7:142791756-142791780; chr7:142791761-142791785; chr7:142791820-142791844; chr7:142791939-142791963; chr7:142791940-142791964; or chr7:142792004-142792028; or (c) chr7:142801104-142801124; chr7:142802103-142802127; or chr7:142802106-142802130.
Embodiment 55 is the engineered cell of any one of embodiments 46-54, wherein the TRBC expression is reduced or eliminated by a genomic editing system that binds to a TRBC target sequence comprising at least 5 contiguous nucleotides within the genomic coordinates chosen from: chr7:142792690-142792714; chr7:142802103-142802127; and chr7:142802106-14280213.
Embodiment 56 is a composition comprising (a) a TRBC guide RNA and optionally (b) an RNA-guided DNA binding agent or a nucleic acid encoding an RNA-guided DNA binding agent, wherein the TRBC guide RNA comprises: i) a guide sequence selected from SEQ ID NOs: 215, 201-214, and 216-265; ii) at least 20, 21, 22, 23, 24, or 25 contiguous nucleotides of a sequence selected from SEQ ID NOs: 215, 201-214, and 216-265; iii) a guide sequence that is at least 95%, 90%, or 85% identical to a sequence selected from SEQ ID NOs: 215, 201-214, and 216-265; iv) a sequence that comprises 10 contiguous nucleotides±10 nucleotides of a genomic coordinate listed in Table 3; v) at least 20, 21, 22, 23, 24, or 25 contiguous nucleotides of a sequence from (iv); or vi) a guide sequence that is at least 95%, 90%, or 85% identical to a sequence selected from (iv).
Embodiment 57 is the composition of embodiment 56, for use in altering a DNA sequence within the TRBC locus in a cell.
Embodiment 58 is the composition of embodiment 56, for use in reducing or eliminating the expression of TRBC protein in a cell.
Embodiment 59 is a method of making an engineered human cell, which has reduced or eliminated surface expression of TRBC protein relative to an unmodified cell, comprising contacting a cell with: (a) a TRBC guide RNA and optionally (b) an RNA-guided DNA binding agent or a nucleic acid encoding an RNA-guided DNA binding agent, wherein the TRBC guide RNA comprises: i) a guide sequence selected from SEQ ID NOs: 215, 201-214, and 216-265; ii) at least 20, 21, 22, 23, 24, or 25 contiguous nucleotides of a sequence selected from SEQ ID NOs: 215, 201-214, and 216-265; iii) a guide sequence that is at least 95%, 90%, or 85% identical to a sequence selected from SEQ ID NOs: 215, 201-214, and 216-265; iv) a sequence that comprises 10 contiguous nucleotides±10 nucleotides of a genomic coordinate listed in Table 3; v) at least 20, 21, 22, 23, 24, or 25 contiguous nucleotides of a sequence from (iv); or vi) a guide sequence that is at least 95%, 90%, or 85% identical to a sequence selected from (iv).
Embodiment 60 is a method of reducing surface expression of TRBC protein in a human cell relative to an unmodified cell, comprising contacting a cell with: (a) a TRBC guide RNA and optionally (b) an RNA-guided DNA binding agent or a nucleic acid encoding an RNA-guided DNA binding agent, wherein the TRBC guide RNA comprises: i) a guide sequence selected from SEQ ID NOs: 215, 201-214, and 216-265; ii) at least 20, 21, 22, 23, 24, or 25 contiguous nucleotides of a sequence selected from SEQ ID NOs: 215, 201-214, and 216-265; iii) a guide sequence that is at least 95%, 90%, or 85% identical to a sequence selected from SEQ ID NOs: 215, 201-214, and 216-265; iv) a sequence that comprises 10 contiguous nucleotides±10 nucleotides of a genomic coordinate listed in Table 3; v) at least 20, 21, 22, 23, 24, or 25 contiguous nucleotides of a sequence from (iv); or vi) a guide sequence that is at least 95%, 90%, or 85% identical to a sequence selected from (iv).
Embodiment 61 is the method or composition of any one of embodiments 56-60, wherein the TRBC guide RNA comprises a guide sequence of any one of SEQ ID NO: 215, 216, 223, 224, 229, 230, 246, 259, and 260.
Embodiment 62 is the method or composition of any one of embodiments 56-61, wherein the TRBC guide RNA comprises a guide sequence of any one of SEQ ID NO: 215, 216, 224, 229, 246, 259, and 260.
Embodiment 63 is the method or composition of any one of embodiments 56-62, wherein the TRBC guide RNA comprises a guide sequence of any one of SEQ ID NOs: 215, 259, and 260.
Embodiment 64 is a population of cells comprising the engineered cells of any one of embodiments 46-55 or the engineered cells produced by use of the composition of embodiment 56 or by the method of any one of embodiments 59-63, wherein greater than about 50%, greater than about 55%, greater than about 60%, greater than about 65%, greater than about 70%, greater than about 75%, greater than about 80%, greater than about 85%, greater than about 90%, greater than about 95%, greater than about 98%, or greater than about 99% of the population of cells are CD3-cells.
Embodiment 65 is a population of cells comprising the engineered cells of any one of embodiments 46-55 or the engineered cells produced by use of the composition of embodiment 56 or by the method of any one of embodiments 59-63, wherein greater than about 50%, greater than about 55%, greater than about 60%, greater than about 65%, greater than about 70%, greater than about 75%, greater than about 80%, greater than about 85%, greater than about 90%, greater than about 95%, greater than about 98%, or greater than about 99% of the population lacks an endogenous T-cell receptor.
Embodiment 66 is a population of cells comprising the engineered cells of any one of embodiments 46-55 or the engineered cells produced by use of the composition of embodiment 56 or by the method of any one of embodiments 59-63, wherein the expression of the TRBC gene in the population has been reduced relative to an unaltered population of the same cell by at least about 50%, at least about 55%, by at least about 60%, at least about 65%, at least about 70%, by at least about 75%, at least about 80%, at least about 85%, by at least about 90%, at least about 95%, at least about 98%, or at least about 99%.
Embodiment 67 is a pharmaceutical composition comprising (a) the engineered cells of any one of embodiments 46-55, or the engineered cells produced by use of the composition of embodiment 56 or by the method of any one of embodiments 59-63, or (b) a population of cells of any one of embodiments 64-66.
Embodiment 68 is an engineered cell, which has reduced or eliminated surface expression of MHC class II relative to an unmodified cell, comprising a genetic modification in the CIITA gene, wherein the genetic modification comprises at least one nucleotide within the genomic coordinates chosen from: (a) chr16:10877363-10907788 or (b) chr16:10906515-10908136.
Embodiment 69 is the engineered cell of embodiment 68, wherein the genetic modification comprises at least one nucleotide within the genomic coordinates chosen from any one of the genomic coordinates listed in Table 4.
Embodiment 70 is the engineered cell of embodiment 68, wherein the genetic modification comprises at least one nucleotide within the genomic coordinates chosen from: (a) chr16:10907504-10907528; chr16:10906643-10906667; chr16:10907508-10907532; chr16:10907539-10907559; chr16:10895658-10895682; chr16:10895668-10895692; chr16:10895750-10895774; chr16:10895753-10895777; chr16:10895754-10895778; chr16:10898684-10898708; chr16:10901529-10901553; chr16:10902121-10902145; chr16:10902701-10902725; chr16:10904726-10904750; chr16:10904760-10904784; chr16:10906493-10906517; chr16:10906515-10906539; chr16:10906631-10906655; chr16:10906636-10906660; chr16:10906770-10906794; chr16:10906788-10906812; chr16:10906789-10906813; chr16:10906816-10906840; chr16:10907148-10907172; chr16:10907254-10907278; chr16:10907331-10907355; chr16:10907477-10907501; chr16:10907497-10907521; chr16:10907503-10907527; chr16:10907574-10907598; or (b) chr16:10906889-10906913 or chr16:10907504-10907528.
Embodiment 71 is the engineered cell of embodiment 68, wherein the genetic modification comprises at least one nucleotide within the genomic coordinates chosen from: chr16:10907504-10907528; chr16:10906643-10906667; chr16:10895658-10895682; chr16:10902701-10902725; chr16:10906493-10906517; chr16:10906631-10906655; chr16:10907477-10907501; chr16:10907497-10907521; or chr16:10907508-10907532.
Embodiment 72 is an engineered cell, which has reduced or eliminated surface expression of MHC class II relative to an unmodified cell, comprising a genetic modification in the CIITA locus, wherein the genetic modification comprises an indel, a C to T substitution, or an A to G substitution within the genomic coordinates chosen from: (a) chr16:10907504-10907528; chr16:10906643-10906667; chr16:10907508-10907532; chr16:10907539-10907559; chr16:10895658-10895682; chr16:10895668-10895692; chr16:10895750-10895774; chr16:10895753-10895777; chr16:10895754-10895778; chr16:10898684-10898708; chr16:10901529-10901553; chr16:10902121-10902145; chr16:10902701-10902725; chr16:10904726-10904750; chr16:10904760-10904784; chr16:10906493-10906517; chr16:10906515-10906539; chr16:10906631-10906655; chr16:10906636-10906660; chr16:10906770-10906794; chr16:10906788-10906812; chr16:10906789-10906813; chr16:10906816-10906840; chr16:10907148-10907172; chr16:10907254-10907278; chr16:10907331-10907355; chr16:10907477-10907501; chr16:10907497-10907521; chr16:10907503-10907527; chr16:10907574-10907598; or (b) chr16:10907504-10907528.
Embodiment 73 is an engineered cell, which has reduced or eliminated surface expression of MHC class II relative to an unmodified cell, comprising a genetic modification in the CIITA locus, wherein the genetic modification comprises an indel, a C to T substitution, or an A to G substitution within the genomic coordinates chosen from: chr16:10907504-10907528; chr16:10906643-10906667; chr16:10895658-10895682; chr16:10902701-10902725; chr16:10906493-10906517; chr16:10906631-10906655; chr16:10907477-10907501; and chr16:10907497-10907521; chr16:10907504-10907528; chr16:10907508-10907532.
Embodiment 74 is the engineered cell of any one of embodiments 68-73, wherein the MHC class II expression is reduced or eliminated by a genomic editing system that binds to a CIITA genomic target sequence comprising at least 5 contiguous nucleotides within the genomic coordinates chosen from (a) chr16:10907504-10907528; chr16:10906643-10906667; chr16:10907508-10907532; chr16:10907539-10907559; chr16:10895658-10895682; chr16:10895668-10895692; chr16:10895750-10895774; chr16:10895753-10895777; chr16:10895754-10895778; chr16:10898684-10898708; chr16:10901529-10901553; chr16:10902121-10902145; chr16:10902701-10902725; chr16:10904726-10904750; chr16:10904760-10904784; chr16:10906493-10906517; chr16:10906515-10906539; chr16:10906631-10906655; chr16:10906636-10906660; chr16:10906770-10906794; chr16:10906788-10906812; chr16:10906789-10906813; chr16:10906816-10906840; chr16:10907148-10907172; chr16:10907254-10907278; chr16:10907331-10907355; chr16:10907477-10907501; chr16:10907497-10907521; chr16:10907503-10907527; chr16:10907574-10907598; or chr16:10907504-10907528.
Embodiment 75 is the engineered cell of any one of embodiments 68-74, wherein the MHC class II expression is reduced or eliminated by a genomic editing system that binds to a CIITA genomic target sequence comprising at least 5 contiguous nucleotides within the genomic coordinates chosen from: chr16:10907504-10907528; chr16:10906643-10906667; chr16:10895658-10895682; chr16:10902701-10902725; chr16:10906493-10906517; chr16:10906631-10906655; chr16:10907477-10907501; chr16:10907497-10907521; or chr16:10907508-10907532.
Embodiment 76 is a composition comprising (a) a CIITA guide RNA and optionally (b) an RNA-guided DNA binding agent or a nucleic acid encoding an RNA-guided DNA binding agent, wherein the CIITA guide RNA comprises: i) a guide sequence selected from SEQ ID NOs: 301, 422, 302-421, and 423-576; or ii) at least 20, 21, 22, 23, 24, or 25 contiguous nucleotides of a sequence selected from SEQ ID NOs: 301, 422, 302-421, and 423-576; or iii) a guide sequence that is at least 95%, 90%, or 85% identical to a sequence selected from SEQ ID NOs: 301, 422, 302-421, and 423-576; or iv) a sequence that comprises 10 contiguous nucleotides±10 nucleotides of a genomic coordinate listed in Table 4; or v) at least 20, 21, 22, 23, 24, or 25 contiguous nucleotides of a sequence from (iv); or vi) a guide sequence that is at least 95%, 90%, or 85% identical to a sequence selected from (iv).
Embodiment 77 is the composition of embodiment 76, for use in altering a DNA sequence within the CIITA gene in a cell.
Embodiment 78 is the composition of embodiment 76, for use in reducing or eliminating the expression of the CIITA in a cell.
Embodiment 79 is a method of making an engineered human cell, which has reduced or eliminated surface expression of MHC class II protein relative to an unmodified cell, comprising contacting a cell with: (a) a CIITA guide RNA and optionally (b) an RNA-guided DNA binding agent or a nucleic acid encoding an RNA-guided DNA binding agent, wherein the CIITA guide RNA comprises: i) a guide sequence selected from SEQ ID NOs: 301, 422, 302-421, and 423-576; or ii) at least 20, 21, 22, 23, 24, or 25 contiguous nucleotides of a sequence selected from SEQ ID NOs: 301, 422, 302-421, and 423-576; or iii) a guide sequence that is at least 95%, 90%, or 85% identical to a sequence selected from SEQ ID NOs: 301, 422, 302-421, and 423-576; or iv) a sequence that comprises 10 contiguous nucleotides±10 nucleotides of a genomic coordinate listed in Table 4; or v) at least 20, 21, 22, 23, 24, or 25 contiguous nucleotides of a sequence from (iv); or vi) a guide sequence that is at least 95%, 90%, or 85% identical to a sequence selected from (iv).
Embodiment 80 is a method of reducing surface expression of MHC class II protein in a human cell relative to an unmodified cell, comprising contacting a cell with: (a) a CIITA guide RNA and optionally (b) an RNA-guided DNA binding agent or a nucleic acid encoding an RNA-guided DNA binding agent, wherein the CIITA guide RNA comprises: i) a guide sequence selected from SEQ ID NOs: 301, 422, 302-421, and 423-576; or ii) at least 20, 21, 22, 23, 24, or 25 contiguous nucleotides of a sequence selected from SEQ ID NOs: 301, 422, 302-421, and 423-576; or iii) a guide sequence that is at least 95%, 90%, or 85% identical to a sequence selected from SEQ ID NOs: 301, 422, 302-421, and 423-576; or iv) a sequence that comprises 10 contiguous nucleotides±10 nucleotides of a genomic coordinate listed in Table 4; or v) at least 20, 21, 22, 23, 24, or 25 contiguous nucleotides of a sequence from (iv); or vi) a guide sequence that is at least 95%, 90%, or 85% identical to a sequence selected from (iv).
Embodiment 81 is the method or composition of any one of embodiments 76-80, wherein the CIITA guide RNA comprises a guide sequence of any one of SEQ ID NOs: 301, 422, 302, 320, 321, 324, 326, 327, 332, 354, 361, 372, 400, 408, 414, 415, 419, 420, 428, 431, 432, 434, 451, 455, 458, 462, 463, 464, 468.
Embodiment 82 is the method or composition of any one of embodiments 76-81, wherein the CIITA guide RNA comprises a guide sequence of any one of SEQ ID NO: 538.
Embodiment 83 is the method or composition of any one of embodiments 76-81, wherein the CIITA guide RNA comprises a guide sequence of any one of SEQ ID NOs: 301, 422, 302, 320, 372, 414, 419, 462, and 463.
Embodiment 84 is a population of cells comprising the engineered cells of any one of embodiments 68-75 or the engineered cells produced by use of the composition of embodiment 76 or by the method of any one of embodiments 79-83, wherein greater than about 50%, greater than about 55%, greater than about 60%, greater than about 65%, greater than about 70%, greater than about 75%, greater than about 80%, greater than about 85%, greater than about 90%, greater than about 95%, greater than about 98%, or greater than about 99% of the population of cells are MHC class II molecules negative as measured by flow cytometry.
Embodiment 85 is a population of cells comprising the engineered cells of any one of embodiments 68-75 or the engineered cells produced by use of the composition of embodiment 76 or by the method of any one of embodiments 79-83, wherein greater than about 50%, greater than about 55%, greater than about 60%, greater than about 65%, greater than about 70%, greater than about 75%, greater than about 80%, greater than about 85%, greater than about 90%, greater than about 95%, greater than about 98%, or greater than about 99% of the population of cells are negative for MHC class II molecules as measured by next generation sequencing (NGS).
Embodiment 86 is a population of cells comprising the engineered cells of any one of embodiments 68-75 or the engineered cells produced by use of the composition of embodiment 76 or by the method of any one of embodiments 79-83, wherein the expression of MHC Class II molecules in the population has been reduced relative to an unaltered population of the same cell by at least about 50%, at least about 55%, by at least about 60%, at least about 65%, at least about 70%, by at least about 75%, at least about 80%, at least about 85%, by at least about 90%, at least about 95%, at least about 98%, or at least about 99%.
Embodiment 87 is a pharmaceutical composition comprising (a) the engineered cells of any one of embodiments 68-75 or the engineered cells produced by use of the composition of embodiment 76 or by the method of any one of embodiments 79-83, or (b) a population of cells of any one of embodiments 84-86.
Embodiment 88 is an engineered cell comprising a genetic modification in the AAVS1 locus, wherein the genetic modification comprises at least one nucleotide within the genomic coordinates chosen from: chr19: 55115151-55116209.
Embodiment 89 is the engineered cell of embodiment 88, wherein the genetic modification comprises at least one nucleotide within the genomic coordinates chosen from any one of the genomic coordinates listed in Table 5.
Embodiment 90 is the engineered cell of embodiment 88, wherein the genetic modification comprises at least one nucleotide within the genomic coordinates chosen from: chr19:55115218-55115242; chr19:55115477-55115501; chr19:55115504-55115528; chr19:55115513-55115537; chr19:55115514-55115538; chr19:55115517-55115541; chr19:55115518-55115542; chr19:55115549-55115573; chr19:55115574-55115598; chr19:55115606-55115630; chr19:55115933-55115957; chr19:55116026-55116050; chr19:55116045-55116069; chr19:55116084-55116108; chr19:55115276-55115300; chr19:55115509-55115533; chr19:55115579-55115603; chr19:55115863-55115887; chr19:55115906-55115930; or chr19:55116006-55116030.
Embodiment 91 is an engineered cell comprising a genetic modification in the AAVS1 gene, wherein the genetic modification comprises an indel, a C to T substitution, or an A to G substitution within the genomic coordinates chosen from: chr19:55115218-55115242; chr19:55115477-55115501; chr19:55115504-55115528; chr19:55115513-55115537; chr19:55115514-55115538; chr19:55115517-55115541; chr19:55115518-55115542; chr19:55115549-55115573; chr19:55115574-55115598; chr19:55115606-55115630; chr19:55115933-55115957; chr19:55116026-55116050; chr19:55116045-55116069; chr19:55116084-55116108; chr19:55115276-55115300; chr19:55115509-55115533; chr19:55115579-55115603; chr19:55115863-55115887; chr19:55115906-55115930; or chr19:55116006-55116030.
Embodiment 92 is the engineered cell of any one of embodiments 88-91, wherein the genetic modification is induced by a genomic editing system that binds to an AAVS1 genomic target sequence comprising at least 5 contiguous nucleotides within the genomic coordinates chosen from chr19:55115218-55115242; chr19:55115477-55115501; chr19:55115504-55115528; chr19:55115513-55115537; chr19:55115514-55115538; chr19:55115517-55115541; chr19:55115518-55115542; chr19:55115549-55115573; chr19:55115574-55115598; chr19:55115606-55115630; chr19:55115933-55115957; chr19:55116026-55116050; chr19:55116045-55116069; chr19:55116084-55116108; chr19:55115276-55115300; chr19:55115509-55115533; chr19:55115579-55115603; chr19:55115863-55115887; chr19:55115906-55115930; or chr19:55116006-55116030.
Embodiment 93 is a composition comprising: a) an AAVS1 guide RNA comprising a guide sequence that i) targets an AAVS1 genomic target sequence; or ii) directs an RNA-guided DNA binding agent to induce a double stranded break (DSB) or a single-stranded break (SSB) in an AAVS1 genomic target sequence comprising at least 10 contiguous nucleotides within the genomic coordinates chosen from: chr19:55115218-55115242; chr19:55115477-55115501; chr19:55115504-55115528; chr19:55115513-55115537; chr19:55115514-55115538; chr19:55115517-55115541; chr19:55115518-55115542; chr19:55115549-55115573; chr19:55115574-55115598; chr19:55115606-55115630; chr19:55115933-55115957; chr19:55116026-55116050; chr19:55116045-55116069; chr19:55116084-55116108; chr19:55115276-55115300; chr19:55115509-55115533; chr19:55115579-55115603; chr19:55115863-55115887; chr19:55115906-55115930; or chr19:55116006-55116030.
Embodiment 94 is a composition comprising: a) an AAVS1 guide RNA (gRNA) and optionally (b) an RNA-guided DNA binding agent or a nucleic acid encoding an RNA-guided DNA binding agent, wherein the AAVS1 guide RNA comprises: i) a guide sequence selected from SEQ ID NOs: 601-774; ii) at least 20, 21, 22, 23, 24, or 25 contiguous nucleotides of a sequence selected from SEQ ID NOs: 601-774; iii) a guide sequence at least 95%, 90%, or 85% identical to a sequence selected from SEQ ID NOs: 601-774; iv) a sequence that comprises 10 contiguous nucleotides±10 nucleotides of a genomic coordinate listed in Table 5; v) at least 20, 21, 22, 23, 24, or 25 contiguous nucleotides of a sequence from (iv); or vi) a guide sequence that is at least 95%, 90%, or 85% identical to a sequence selected from (iv).
Embodiment 95 is a method of making an engineered human cell comprising contacting a cell with: (a) an AAVS1 guide RNA and optionally (b) an RNA-guided DNA binding agent or a nucleic acid encoding an RNA-guided DNA binding agent, wherein the AAVS1 guide RNA comprises: i) a guide sequence selected from SEQ ID NOs: 601-774; ii) at least 20, 21, 22, 23, 24, or 25 contiguous nucleotides of a sequence selected from SEQ ID NOs: 601-774; iii) a guide sequence at least 95%, 90%, or 85% identical to a sequence selected from SEQ ID NOs: 601-774; iv) a sequence that comprises 10 contiguous nucleotides±10 nucleotides of a genomic coordinate listed in Table 5; v) at least 20, 21, 22, 23, or 24, contiguous nucleotides of a sequence from (iv); or vi) a guide sequence that is at least 95%, 90%, or 85% identical to a sequence selected from (iv).
Embodiment 96 is the method or composition of any one of embodiments 93-95, wherein the AAVS1 guide RNA comprises a guide sequence of any one of SEQ ID NOs: 611, 620, 622, 626, 627, 628, 629, 632, 633, 634, 656, 659, 660, 661, 673, 691, 692, 730, 734, and 746.
Embodiment 97 is the engineered cell, population of cells, pharmaceutical composition, or method of any one of embodiments 1-96, wherein the genetic modification comprises at least 2, at least 3, at least 4, at least 5, at least 6, at least 7, at least 8, at least 9, or at least 10 contiguous nucleotides within the genomic coordinates.
Embodiment 98 is the engineered cell, population of cells, pharmaceutical composition, or method of any one of embodiments 1-97, wherein the genetic modification comprises at least 5, 6, 7, 8, 9, or 10 contiguous nucleotides within the genomic coordinates.
Embodiment 99 is the engineered cell, population of cells, pharmaceutical composition, or method of any one of embodiments 1-98, wherein the genomic target sequence comprises at least 10 contiguous nucleotides within the genomic coordinates.
Embodiment 100 is the engineered cell, population of cells, pharmaceutical composition, or method of any one of embodiments 1-99, wherein the genomic target sequence comprises at least 15 contiguous nucleotides within the genomic coordinates.
Embodiment 101 is the engineered cell, population of cells, pharmaceutical composition, or method of any one of embodiments 1-100, wherein the genomic target sequence comprises at least 17, 18, 19, 20, 21, 22, 23, or 24 contiguous nucleotides within the genomic coordinates.
Embodiment 102 is the engineered cell, population of cells, pharmaceutical composition, or method of any one of embodiments 1-101, wherein the genetic modification comprises an indel.
Embodiment 103 is the engineered cell of any one of embodiments 1-102, wherein the genetic modification comprises an insertion of a heterologous coding sequence.
Embodiment 104 is the engineered cell, population of cells, pharmaceutical composition, or method of any one of embodiments 1-103, wherein the genetic modification comprises at least one A to G substitution within the genomic coordinates.
Embodiment 105 is the engineered cell, population of cells, pharmaceutical composition, or method of any one of embodiments 1-104, wherein the genetic modification comprises at least one C to T substitution within the genomic coordinates.
Embodiment 106 is the engineered cell, population of cells, pharmaceutical composition, or method of any one of embodiments 1-105, wherein the cell has a genetic modification in the CIITA gene.
Embodiment 107 is the engineered cell, population of cells, pharmaceutical composition, or method of any one of embodiments 1-106, wherein the cell has reduced expression of TRAC protein on the surface of the cell.
Embodiment 108 is the engineered cell, population of cells, pharmaceutical composition, or method of any one of embodiments 1-107, wherein the cell has reduced expression of TRBC protein on the surface of the cell.
Embodiment 109 is the engineered cell, population of cells, pharmaceutical composition, or method of any one of embodiments 1-108, wherein the cell has reduced expression of MHC class II molecules on the surface of the cell.
Embodiment 110 is the engineered cell, population of cells, pharmaceutical composition, or method of any one of embodiments 1-109, wherein the engineered cell is an immune cell.
Embodiment 111 is the engineered cell, population of cells, pharmaceutical composition, or method of any one of embodiments 1-110, wherein the cell is a primary cell.
Embodiment 112 is the engineered cell, population of cells, pharmaceutical composition, or method of any one of embodiments 1-111, wherein the engineered cell is a monocyte, macrophage, mast cell, dendritic cell, or granulocyte.
Embodiment 113 is the engineered cell, population of cells, pharmaceutical composition, or method of any one of embodiments 1-112, wherein the engineered cell is a lymphocyte.
Embodiment 114 is the engineered cell, population of cells, pharmaceutical composition, or method of any one of embodiments 1-113, wherein the cell is a T cell.
Embodiment 115 is the engineered cell, population of cells, pharmaceutical composition, or method of any one of embodiments 1-114, wherein the cell is a CD8+ T cell.
Embodiment 116 is the engineered cell, population of cells, pharmaceutical composition, or method of any one of embodiments 1-114, wherein the cell is a CD4+ T cell.
Embodiment 117 is the engineered cell, population of cells, pharmaceutical composition, or method of any one of embodiments 1-113, wherein the cell is a natural killer (NK) cell.
Embodiment 118 is the engineered cell, population of cells, pharmaceutical composition, or method of any one of embodiments 1-113, wherein the cell is a macrophage.
Embodiment 119 is the engineered cell, population of cells, pharmaceutical composition, or method of any one of embodiments 1-113, wherein the cell is a B cell.
Embodiment 120 is the engineered cell, population of cells, pharmaceutical composition, or method of any one of embodiments 1-113, wherein the cell is a plasma B cell.
Embodiment 121 is the engineered cell, population of cells, pharmaceutical composition, or method of any one of embodiments 1-113, wherein the cell is memory B cell.
Embodiment 122 is the engineered cell, population of cells, pharmaceutical composition, or method of any one of embodiments 1-109, wherein the cell is a stem cell.
Embodiment 123 is the engineered cell, population of cells, pharmaceutical composition, or method of any one of embodiments 1-109, wherein the cell is a progenitor cell.
Embodiment 124 is the engineered cell, population of cells, pharmaceutical composition, or method of any one of embodiments 1-109, wherein the cell is an HSC.
Embodiment 125 is the engineered cell, population of cells, pharmaceutical composition, or method of any one of embodiments 1-109, wherein the cell is an iPSC.
Embodiment 126 is the engineered cell, population of cells, pharmaceutical composition, or method of any one of embodiments 1-121, wherein the cell is an activated cell.
Embodiment 127 is the engineered cell, population of cells, pharmaceutical composition, or method of any one of embodiments 1-121, wherein the cell is a non-activated cell.
Embodiment 128 is the population of embodiment 18 or pharmaceutical composition of embodiment 19, wherein the population of cells is at least 65%, at least 70%, at least 75%, at least 80%, at least 85%, at least 90%, at least 95%, at least 96%, at least 97%, at least 98%, or at least 99% HLA-A negative as measured by flow cytometry.
Embodiment 129 is the population of any one of embodiments 42-44 and 64-66 or pharmaceutical composition of embodiment 45 or 67, wherein the population of cells is at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or at least 99.5% endogenous TCR protein negative as measured by flow cytometry.
Embodiment 130 is the population of any one of embodiments 84-86 or pharmaceutical composition of embodiment 87, wherein the population of cells is at least 65%, at least 70%, at least 75%, at least 80%, at least 85%, at least 90%, at least 95%, at least 96%, at least 97%, at least 98%, at least 99%, or at least 99.5% HLA-DP, DQ, DR negative as measured by flow cytometry.
Embodiment 131 is the population of embodiment 18 or pharmaceutical composition of embodiment 19, wherein at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or at least 99.5% of the population of cells comprises the genetic modification in the HLA-A gene as measured by next-generation sequencing (NGS).
Embodiment 132 is the population of any one of embodiments 42-44 or pharmaceutical composition of embodiment 45, wherein at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% of the population of cells comprises the genetic modification in the TRAC gene as measured by next-generation sequencing (NGS).
Embodiment 133 is the population of any one of embodiments 64-66 or pharmaceutical composition of embodiment 67, wherein at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% of the population of cells comprises the genetic modification in the TRBC gene as measured by next-generation sequencing (NGS).
Embodiment 134 is the population of any one of embodiments 84-86 or pharmaceutical composition of embodiment 87, wherein at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% of the population of cells comprises the genetic modification in the CIITA gene as measured by next-generation sequencing (NGS).
Embodiment 135 is the engineered cell, population of cells, pharmaceutical composition, or method of any one of embodiments 1-134, wherein the cell is an allogeneic cell.
Embodiment 136 is the engineered cell, population of cells, pharmaceutical composition, or method of any one of embodiments 1-135, wherein the cell is a primary cell.
Embodiment 137 is the engineered cell, population of cells, pharmaceutical composition, or method of any one of the embodiments 1-136, wherein the cell is a CD4+ T cell.
Embodiment 138 is the engineered cell, population of cells, pharmaceutical composition, or method of any one of embodiments 1-136, wherein the cell is a CD8+ T cell.
Embodiment 139 is the engineered cell, population of cells, pharmaceutical composition, or method of any one of embodiments 1-136, wherein the cell is a memory T cell.
Embodiment 140 is the engineered cell, population of cells, pharmaceutical composition, or method of any one of embodiments 1-136, wherein the cell is a B cell.
Embodiment 141 is the engineered cell, population of cells, pharmaceutical composition, or method of any one of embodiments 1-136, wherein the cell is a plasma B cell.
Embodiment 142 is the engineered cell, population of cells, pharmaceutical composition, or method of any one of embodiments 1-136, wherein the cell is a memory B cell.
Embodiment 143 is the engineered cell, population of cells, pharmaceutical composition, or method of any one of embodiments 1-136, wherein the cell is a natural killer (NK) cell.
Embodiment 144 is the engineered cell, population of cells, pharmaceutical composition, or method of any one of embodiments 1-136, wherein the cell is a macrophage.
Embodiment 145 is the engineered cell, population of cells, pharmaceutical composition, or method of any one of embodiments 1-136, wherein the cell is stem cell.
Embodiment 146 is the engineered cell, population of cells, pharmaceutical composition, or method of any one of embodiments 1-136, wherein the cell is a pluripotent stem cell (PSC).
Embodiment 147 is the engineered cell, population of cells, pharmaceutical composition, or method of any one of embodiments 1-136, wherein the cell is a hematopoietic stem cell (HSC).
Embodiment 148 is the engineered cell, population of cells, pharmaceutical composition, or method of any one of embodiments 1-136, wherein the cell is an induced pluripotent stem cell (iPSC).
Embodiment 149 is the engineered cell, population of cells, pharmaceutical composition, or method of any one of embodiments 1-136, wherein the cell is a mesenchymal stem cell (MSC).
Embodiment 150 is the engineered cell, population of cells, pharmaceutical composition, or method of any one of embodiments 1-136, wherein the cell is a neural stem cell (NSC).
Embodiment 151 is the engineered cell, population of cells, pharmaceutical composition, or method of any one of embodiments 1-136, wherein the cell is a limbal stem cell (LSC).
Embodiment 152 is the engineered cell, population of cells, pharmaceutical composition, or method of any one of embodiments 1-136, wherein the cell is a progenitor cell, e.g. an endothelial progenitor cell or a neural progenitor cell.
Embodiment 153 is the engineered cell, population of cells, pharmaceutical composition, or method of any one of embodiments 1-136, wherein the cell is a tissue-specific primary cell.
Embodiment 154 is the engineered cell, population of cells, pharmaceutical composition, or method of any one of embodiments 1-136, wherein the cell is a chosen from: chondrocyte, myocyte, and keratinocyte.
Embodiment 155 is the engineered cell, population of cells, pharmaceutical composition, or method of any one of embodiments 1-136, wherein the cell is an activated cell.
Embodiment 156 is the engineered cell, population of cells, pharmaceutical composition, or method of any one of embodiments 1-136, wherein the cell is a non-activated cell.
Embodiment 157 is the engineered cell, population of cells, pharmaceutical composition, or method of any one of embodiments 1-156, wherein the cells are engineered with a genomic editing system.
Embodiment 158 is the engineered cell, population of cells, pharmaceutical composition, or method of any one of embodiments 7-157, wherein the genomic editing system comprises an RNA-guided DNA-binding agent or a nucleic acid encoding an RNA-guided DNA binding agent.
Embodiment 159 is the engineered cell, population of cells, pharmaceutical composition, or method of embodiment 158, wherein the RNA-guided DNA-binding agent or the RNA-guided DNA-binding agent encoded by the nucleic acid is N. meningitidis Cas9 (NmeCas9).
Embodiment 160 is the engineered cell, population of cells, pharmaceutical composition, or method of embodiment 159, wherein the NmeCas9 is Nme1Cas9, Nme2Cas9, or Nme3Cas9.
Embodiment 161 is the engineered cell, population of cells, pharmaceutical composition, or method of any one of embodiments 158-160, wherein the RNA-guided DNA-binding agent or the RNA-guided DNA-binding agent encoded by the nucleic acid has double-stranded endonuclease activity.
Embodiment 162 is the engineered cell, population of cells, pharmaceutical composition, or method of any one of embodiments 158-161, wherein the RNA-guided DNA-binding agent or the RNA-guided DNA-binding agent encoded by the nucleic acid has nickase activity.
Embodiment 163 is the engineered cell, population of cells, pharmaceutical composition, or method of any one of embodiments 158-161, wherein the RNA-guided DNA-binding agent or the RNA-guided DNA-binding agent encoded by the nucleic acid comprises a dCas9 DNA binding domain.
Embodiment 164 is the engineered cell, population of cells, pharmaceutical composition, or method of any one of embodiments 158-160, wherein the RNA-guided DNA-binding agent or nucleic acid encoding the RNA-guided DNA binding agent is a A to G base editor.
Embodiment 165 is the engineered cell, population of cells, pharmaceutical composition, or method of any one of embodiments 158-160, wherein the RNA-guided DNA-binding agent or nucleic acid encoding the RNA-guided DNA binding agent is a C to T base editor.
Embodiment 166 is the engineered cell, population of cells, pharmaceutical composition, or method of any one of embodiments 158-165, wherein the RNA-guided DNA-binding agent or the RNA-guided DNA-binding agent encoded by the nucleic acid comprises a deaminase region.
Embodiment 167 is the engineered cell of embodiment 158, wherein the RNA-guided DNA-binding agent or the RNA-guided DNA-binding agent encoded by the nucleic acid comprises an APOBEC3A deaminase (A3A) and an N. meningitidis Cas9 nickase.
Embodiment 168 is the engineered cell, population of cells, pharmaceutical composition, or method of any one of embodiments 1-167, wherein the guide RNA is provided to the cell in a vector.
Embodiment 169 is the engineered cell, population of cells, pharmaceutical composition, or method of any one of embodiments 1-168, wherein the RNA-guided DNA binding agent is provided to the cell in a vector, optionally in the same vector as the guide RNA.
Embodiment 170 is the engineered cell, population of cells, pharmaceutical composition, or method of any one of embodiments 1-169, wherein the exogenous nucleic acid is provided to the cell in a vector.
Embodiment 171 is the engineered cell, population of cells, pharmaceutical composition, or method of any one of embodiments 168-170, wherein the vector is a viral vector.
Embodiment 172 is the engineered cell, population of cells, pharmaceutical composition, or method of any one of embodiments 168-170, wherein the vector is a non-viral vector.
Embodiment 173 is the engineered cell, population of cells, pharmaceutical composition, or method of any one of embodiments 168-171, wherein the vector is a lentiviral vector.
Embodiment 174 is the engineered cell, population of cells, pharmaceutical composition, or method of any one of embodiments 168-171, wherein the vector is a retroviral vector.
Embodiment 175 is the engineered cell, population of cells, pharmaceutical composition, or method of any one of embodiments 168-171, wherein the vector is an AAV.
Embodiment 176 is the engineered cell, population of cells, pharmaceutical composition, or method of any one of embodiments 1-175, wherein the guide RNA is provided to the cell in a lipid nucleic acid assembly composition, optionally in the same lipid nucleic acid assembly composition as an RNA-guided DNA binding agent.
Embodiment 177 is the engineered cell, population of cells, pharmaceutical composition, or method of any one of embodiments 1-176, wherein the exogenous nucleic acid is provided to the cell in a lipid nucleic acid assembly composition.
Embodiment 178 is the engineered cell, population of cells, pharmaceutical composition, or method of embodiment 176 or 177, wherein the lipid nucleic acid assembly composition is a lipid nanoparticle (LNP).
Embodiment 179 is the engineered cell, population of cells, pharmaceutical composition, or method of any one of embodiments 1-178, wherein the exogenous nucleic acid is integrated into the genome of the cell.
Embodiment 180 is the engineered cell, population of cells, pharmaceutical composition, or method of any one of embodiments 1-179, wherein the exogenous nucleic acid is integrated into the genome of the cell by homologous recombination (HR).
Embodiment 181 is the engineered cell, population of cells, pharmaceutical composition, or method of any one of embodiments 1-180, wherein the exogenous nucleic acid is integrated into a safe harbor locus in the genome of the cell.
Embodiment 182 is the engineered cell, population of cells, pharmaceutical composition, or method of any one of embodiments 1-181, wherein the guide RNA is a single guide RNA.
Embodiment 183 is the engineered cell, population of cells, pharmaceutical composition, or method of embodiment 182, wherein the single guide RNA comprises the nucleotide sequence of SEQ ID NO: 900 3′ to the guide sequence.
Embodiment 184 is the engineered cell, population of cells, pharmaceutical composition, or method of embodiment 182 or 183, wherein the single guide RNA comprises a guide region and a conserved region, wherein the conserved region comprising one or more of: (a) a shortened repeat/anti-repeat region, wherein the shortened repeat/anti-repeat region lacks 2-24 nucleotides, wherein (i) one or more of nucleotides 37-48 and 53-64 is deleted and optionally one or more of nucleotides 37-64 is substituted relative to SEQ ID NO: 900; and (ii) nucleotide 36 is linked to nucleotide 65 by at least 2 nucleotides; or (b) a shortened hairpin 1 region, wherein the shortened hairpin 1 lacks 2-10, optionally 2-8 nucleotides, wherein (i) one or more of nucleotides 82-86 and 91-95 is deleted and optionally one or more of positions 82-96 is substituted relative to SEQ ID NO: 900; and (ii) nucleotide 81 is linked to nucleotide 96 by at least 4 nucleotides; or (c) a shortened hairpin 2 region, wherein the shortened hairpin 2 lacks 2-18, optionally 2-16 nucleotides, wherein (i) one or more of nucleotides 113-121 and 126-134 is deleted and optionally one or more of nucleotides 113-134 is substituted relative to SEQ ID NO: 900; and (ii) nucleotide 112 is linked to nucleotide 135 by at least 4 nucleotides; wherein one or both nucleotides 144-145 are optionally deleted relative to SEQ ID NO: 900; wherein at least 10 nucleotides are modified nucleotides.
Embodiment 185 is the engineered cell, population of cells, pharmaceutical composition, or method of any one of embodiments 1-184, wherein the guide RNA comprises at least one modification.
Embodiment 186 is the engineered cell, population of cells, pharmaceutical composition, or method of embodiment 185, wherein the modification comprises a modified nucleotide selected from a 2′—O-methyl (2′—OMe) modified nucleotide, 2′—O-(2-methoxyethyl) (2′—O-moe) modified nucleotide, a 2′-fluoro (2′—F) modified nucleotide, a phosphorothioate (PS) linkage between nucleotides, or an inverted abasic modified nucleotide.
Embodiment 187 is the engineered cell, population of cells, pharmaceutical composition, or method of any one of embodiments 1-186, wherein the gRNA comprises a 5′ end modification, a modification in the repeat/anti-repeat region, a modification in the hairpin 1 region, a modification in the hairpin 2 region, or a 3′ end modification.
Embodiment 188 is the engineered cell, population of cells, pharmaceutical composition, or method of embodiment 187, wherein the 5′ end modification comprises at least one PS linkage, and wherein one or more of: i. there is one PS linkage, and the linkage is between the first and second nucleotides; ii. there are two PS linkages between the first three nucleotides; iii. there are PS linkages between any one or more of the first four nucleotides; and iv. there are PS linkages between any one or more of the first five nucleotides.
Embodiment 189 is the engineered cell, population of cells, pharmaceutical composition, or method of embodiment 187 or 188, wherein the 5′ end modification further comprises at least one 2′—OMe, 2′—O-moe, inverted abasic, or 2′—F modified nucleotide.
Embodiment 190 is the engineered cell, population of cells, pharmaceutical composition, or method of any one of embodiments 187-189, wherein the 5′ end modification comprises: i. a modification of one or more of the first 1-4 nucleotides, wherein the modification is a PS linkage, inverted abasic nucleotide, 2′—OMe, 2′—O-moe, or 2′—F; ii. a modification to the first nucleotide with 2′—OMe, 2′—O-moe, or 2′—F, and an optional one or two PS linkages to the next nucleotide or the first nucleotide of the 3′ tail; iii. a modification to the first or second nucleotide with 2′—OMe, 2′—O-moe, or 2′—F, and optionally one or more PS linkages; iv. a modification to the first, second, or third nucleotides with 2′—OMe, 2′—O-moe, or 2′—F, and optionally one or more PS linkages; or v. a modification to the first, second, third, or forth nucleotides with 2′—OMe, 2′—O-moe, or 2′—F, and optionally one or more PS linkages.
Embodiment 191 is the engineered cell, population of cells, pharmaceutical composition, or method of any one of embodiments 187-180, wherein the 3′ end modification comprises at least one PS linkage, and wherein one or more of: i. there is one PS linkage, and the linkage is between the last and second to last nucleotides; ii. there are two PS linkages between the last three nucleotides; and iii. there are PS linkages between any one or more of the last four nucleotides.
Embodiment 192 is the engineered cell, population of cells, pharmaceutical composition, or method of 191, wherein the 3′ end modification further comprises at least one 2′-OMe, 2′—O-moe, inverted abasic, or 2′—F modified nucleotide.
Embodiment 193 is the engineered cell, population of cells, pharmaceutical composition, or method of 192, wherein the 3′ end modification comprises: i. a modification of one or more of the last 1-4 nucleotides, wherein the modification is a PS linkage, inverted abasic nucleotide, 2′—OMe, 2′—O-moe, or 2′—F; ii. a modification to the last nucleotide with 2′—OMe, 2′-O-moe, or 2′—F, and an optional one or two PS linkages to the next nucleotide or the first nucleotide of the 3′ tail; iii. a modification to the last or second to last nucleotide with 2′—OMe, 2′—O-moe, or 2′—F, and optionally one or more PS linkages; iv. a modification to the last, second to last, or third to last nucleotides with 2′—OMe, 2′—O-moe, or 2′—F, and optionally one or more PS linkages; or v. a modification to the last, second to last, third to last, or fourth to last nucleotides with 2′—OMe, 2′—O-moe, or 2′—F, and optionally one or more PS linkages.
Embodiment 194 is the engineered cell, population of cells, pharmaceutical composition, or method of 193, further comprising a 3′ tail comprising a 2′—O-Me modified nucleotide.
Embodiment 195 is the engineered cell, population of cells, pharmaceutical composition, or method of any one of embodiments 1-194, wherein the guide RNA comprises a 5′ end modification or a 3′ end modification.
Embodiment 196 is the method or composition of any one of embodiments 1-195, wherein the guide RNA comprises:
   the guide sequence, wherein the guide sequence comprises: 2′—O-Me modified nucleotides at the first four nucleotides 1-4; PS linkages between nucleotides 1-2, 2-3, and 3-4; and 2′—O-Me modified nucleotides at nucleotides 5, 8, 9, 11, 13, 18, and 22 of the guide sequence; a shortened repeat/anti-repeat region, wherein nucleotides 38-48 and 53-63 are deleted relative to SEQ ID NO: 900, comprising: 2′—O-Me modified nucleotides at nucleotides 25, 29, 30, 31, 32, 37, 49-52, 64, 65, 69, 70, and 73; a shortened hairpin 1 region, wherein nucleotides 86 and 91 are deleted relative to SEQ ID NO: 900, comprising: 2′—O-Me modified nucleotides at nucleotides 80, 81, 83, 84, 85, 87-90, 92-94, and 99; 2′—O-Me modified nucleotide at nucleotide 101 between the shortened hairpin 1 region and the shortened hairpin 2 region; a shortened hairpin 2 region, wherein nucleotides 112-120 and 127-135 are deleted relative to SEQ ID NO: 900, comprising: 2′—O-Me modified nucleotides at nucleotides 104, 110, 111, 122-125, 142, and 143, PS linkages between nucleotides 141-142 and 142-143, wherein one or both nucleotides 144-145 are optionally deleted relative to SEQ ID NO: 900.
Embodiment 197 is the method or composition of any one of embodiments 1-196, wherein the guide RNA comprises the modified nucleotides of any one of SEQ ID NOs: 904-909, 911, 995-997, and 1081-1089.
Embodiment 198 is the method or composition of any one of embodiments 1-197, wherein the guide RNA comprises the modified nucleotides of SEQ ID NO: 995.
Embodiment 199 is the method or composition of any one of embodiments 1-198, wherein the guide RNA comprises the modified nucleotides of SEQ ID NO: 1083.
Embodiment 200 is the method or composition of any one of embodiments 1-199, wherein the guide RNA is modified according to the pattern of any one of SEQ ID NOs: 904-909, 911, and 995-997, wherein each N in the pattern is any natural or non-natural nucleotide wherein the N's are collectively any one of the guide sequences of Tables 1-5.
Embodiment 201 is the method or composition of any one of embodiments 1-200, wherein the guide RNA is modified according to the pattern of SEQ ID NO: 995, wherein each N in the pattern is any natural or non-natural nucleotide wherein the N's are collectively any one of the guide sequences of Tables 1-5.
Embodiment 202 is a method of administering the engineered cell, population of cells, pharmaceutical composition of any one of embodiments 1-10, 18-28, 42-55, 64-75, 84-92, and 97-195 to a subject in need thereof.
Embodiment 203 is a method of administering the engineered cell, population of cells, or pharmaceutical composition of any one of embodiments 1-10, 18-28, 42-55, 64-75, 84-92, and 97-195 to a subject as an adoptive cell transfer (ACT) therapy.
Embodiment 204 is a method of treating a disease or disorder comprising administering the engineered cell, population of cells, or pharmaceutical composition of any one of embodiments 1-10, 18-28, 42-55, 64-75, 84-92, and 97-195 to a subject in need thereof.
Embodiment 205 is the engineered cell, population of cells, pharmaceutical composition, or method of any one of embodiments 1-10, 18-28, 42-55, 64-75, 84-92, and 97-195, for use in administering to a subject as an adoptive cell transfer (ACT) therapy.
Embodiment 206 is the engineered cell, population of cells, pharmaceutical composition, or method of any one of embodiments 1-10, 18-28, 42-55, 64-75, 84-92, and 97-195, for use in treating a subject with cancer.
Embodiment 207 is the engineered cell, population of cells, pharmaceutical composition, or method of any one of embodiments 1-10, 18-28, 42-55, 64-75, 84-92, and 97-195, for use in treating a subject with an infectious disease.
Embodiment 208 is the engineered cell, population of cells, pharmaceutical composition, or method of any one of embodiments 1-10, 18-28, 42-55, 64-75, 84-92, and 97-195, for use in treating a subject with an autoimmune disease.

### I. Definitions

Unless stated otherwise, the following terms and phrases as used herein are intended to have the following meanings:

The term “or combinations thereof” as used herein refers to all permutations and combinations of the listed terms preceding the term. For example, “A, B, C, or combinations thereof” is intended to include at least one of: A, B, C, AB, AC, BC, or ABC, and if order is important in a particular context, also BA, CA, CB, ACB, CBA, BCA, BAC, or CAB. Continuing with this example, expressly included are combinations that contain repeats of one or more item or term, such as BB, AAA, AAB, BBC, CBBA, CABA, and so forth. The skilled artisan will understand that typically there is no limit on the number of items or terms in any combination, unless otherwise apparent from the context.

As used herein, the term “kit” refers to a packaged set of related components, such as one or more polynucleotides or compositions and one or more related materials such as delivery devices (e.g., syringes), solvents, solutions, buffers, instructions, or desiccants.

An “allogeneic” cell, as used herein, refers to a cell originating from a donor subject of the same species as a recipient subject, wherein the donor subject and recipient subject have genetic dissimilarity, e.g., genes at one or more loci that are not identical. Thus, e.g., a cell is allogeneic with respect to the subject to be administered the cell. As used herein, a cell that is removed or isolated from a donor, that will not be re-introduced into the original donor, is considered an allogeneic cell.

An “autologous” cell, as used herein, refers to a cell derived from the same subject to whom the material will later be re-introduced. Thus, e.g., a cell is considered autologous if it is removed from a subject and it will then be re-introduced into the same subject.

“CIITA” or “CIITA” or “C2TA,” as used herein, refers to the nucleic acid sequence or protein sequence of “class II major histocompatibility complex transactivator;” the human gene has accession number NC_000016.10 (range 10866208 . . . 10941562), reference GRCh38.p13. The CIITA protein in the nucleus acts as a positive regulator of MHC class II gene transcription and is required for MHC class II protein expression.

As used herein, “MHC” or “MHC molecule(s)” or “MHC protein” or “MHC complex(es),” refers to a major histocompatibility complex molecule (or plural), and includes e.g., MHC class I and MHC class II molecules. In humans, MHC molecules are referred to as “human leukocyte antigen” complexes or “HLA molecules” or “HLA protein.” The use of terms “MHC” and “HLA” are not meant to be limiting; as used herein, the term “MHC” may be used to refer to human MHC molecules, i.e., HLA molecules. Therefore, the terms “MHC” and “HLA” are used interchangeably herein.

The term “HLA-A,” as used herein in the context of HLA-A protein, refers to the MHC class I protein molecule, which is a heterodimer consisting of a heavy chain (encoded by the HLA-A gene) and a light chain (i.e., beta-2 microglobulin). The term “HLA-A” or “HLA-A gene,” as used herein in the context of nucleic acids refers to the gene encoding the heavy chain of the HLA-A protein molecule. The HLA-A gene is also referred to as “HLA class I histocompatibility, A alpha chain;” the human gene has accession number NC_000006.12 (29942532 . . . 29945870). The HLA-A gene is known to have thousands of different versions (also referred to as “alleles”) across the population (and an individual may receive two different alleles of the HLA-A gene). A public database for HLA-A alleles, including sequence information, may be accessed at IPD-IMGT/HLA: https://www.ebi.ac.uk/ipd/imgt/hla/. All alleles of HLA-A are encompassed by the terms “HLA-A” and “HLA-A gene.”

“HLA-B” as used herein in the context of nucleic acids refers to the gene encoding the heavy chain of the HLA-B protein molecule. The HLA-B is also referred to as “HLA class I histocompatibility, B alpha chain;” the human gene has accession number NC_000006.12 (31353875 . . . 31357179).

“HLA-C” as used herein in the context of nucleic acids refers to the gene encoding the heavy chain of the HLA-C protein molecule. The HLA-C is also referred to as “HLA class I histocompatibility, C alpha chain;” the human gene has accession number NC_000006.12 (31268749 . . . 31272092).

The term “TRAC,” as used herein in the context of TRAC protein, refers to the T-cell receptor a-chain. “TRAC” as used herein in the context of nucleic acids refers to the gene encoding the T-cell receptor a-chain. A human wild-type TRAC sequence is available at NCBI Gene ID: 28755; Ensembl: ENSG00000277734. T Cell Receptor Alpha Constant, TCRA, IMD7, TRCA and TRA are gene synonyms for TRAC.

The term “TRBC” (or “TRBC1/2”) is used to refer to the nucleic acid sequence or amino acid sequence of the “T-cell receptor 0-chain”, e.g., TRBC1 and TRBC2. The terms “TRBC1” and “TRBC2,” as used herein in the context of TRBC proteins, refer to two homologous proteins that comprise the T-cell receptor 0-chain. “TRBC1” and “TRBC2” as used herein in the context of nucleic acids refers to the genes encoding the T-cell receptor 0-chain. A human wild-type TRBC1 sequence is available at NCBI Gene ID: 28639; Ensembl: ENSG00000211751. T Cell Receptor Beta Constant, V_segment Translation Product, BV05S1J2.2, TCRBC1, and TCRB are gene synonyms for TRBC1. A human wild-type TRBC2 sequence is available at NCBI Gene ID: 28638; Ensembl: ENSG00000211772. T Cell Receptor Beta Constant, V_segment Translation Product, and TCRBC2 are gene synonyms for TRBC2.

As used herein, the term “AAVS1” refers to the genomic location at chr19:50900000-58617616 according to hg38.

As used herein, the term “within the genomic coordinates” includes the boundaries of the genomic coordinate range given. For example, if chr6:29942854-chr6:29942913 is given, the coordinates chr6:29942854-chr6:29942913 are encompassed. Throughout this application, the referenced genomic coordinates are based on genomic annotations in the GRCh38 (also referred to as hg38) assembly of the human genome from the Genome Reference Consortium, available at the National Center for Biotechnology Information website. Tools and methods for converting genomic coordinates between one assembly and another are known in the art and can be used to convert the genomic coordinates provided herein to the corresponding coordinates in another assembly of the human genome, including conversion to an earlier assembly generated by the same institution or using the same algorithm (e.g., from GRCh38 to GRCh37), and conversion of an assembly generated by a different institution or algorithm (e.g., from GRCh38 to NCBI33, generated by the International Human Genome Sequencing Consortium). Available methods and tools known in the art include, but are not limited to, NCBI Genome Remapping Service, available at the National Center for Biotechnology Information website, UCSC LiftOver, available at the UCSC Genome Brower website, and Assembly Converter, available at the Ensembl.org website.

As used herein, the term “homozygous” refers to having two identical alleles of a particular gene.

As used herein, the term “subject” is intended to include living organisms in which an immune response can be elicited, including e.g., mammals, primates, humans.

“Polynucleotide” and “nucleic acid” are used herein to refer to a multimeric compound comprising nucleosides or nucleoside analogs which have nitrogenous heterocyclic bases or base analogs linked together along a backbone, including conventional RNA, DNA, mixed RNA-DNA, and polymers that are analogs thereof. A nucleic acid “backbone” can be made up of a variety of linkages, including one or more of sugar-phosphodiester linkages, peptide-nucleic acid bonds (“peptide nucleic acids” or PNA; PCT No. WO 95/32305), phosphorothioate linkages, methylphosphonate linkages, or combinations thereof. Sugar moieties of a nucleic acid can be ribose, deoxyribose, or similar compounds with substitutions, e.g., 2′ methoxy or 2′ halide substitutions. Nitrogenous bases can be conventional bases (A, G, C, T, U), analogs thereof (e.g., modified uridines such as 5-methoxyuridine, pseudouridine, or N1-methylpseudouridine, or others); inosine; derivatives of purines or pyrimidines (e.g., N⁴-methyl deoxyguanosine, deaza- or aza-purines, deaza- or aza-pyrimidines, pyrimidine bases with substituent groups at the 5 or 6 position (e.g., 5-methylcytosine), purine bases with a substituent at the 2, 6, or 8 positions, 2-amino-6-methylaminopurine, O⁶-methylguanine, 4-thio-pyrimidines, 4-amino-pyrimidines, 4-dimethylhydrazine-pyrimidines, and O⁴-alkyl-pyrimidines; U.S. Pat. No. 5,378,825 and PCT No. WO 93/13121). For general discussion see The Biochemistry of the Nucleic Acids 5-36, Adams et al., ed., 11^{th} ed., 1992). Nucleic acids can include one or more “abasic” residues where the backbone includes no nitrogenous base for position(s) of the polymer (U.S. Pat. No. 5,585,481). A nucleic acid can comprise only conventional RNA or DNA sugars, bases and linkages, or can include both conventional components and substitutions (e.g., conventional bases with 2′ methoxy linkages, or polymers containing both conventional bases and one or more base analogs). Nucleic acid includes “locked nucleic acid” (LNA), an analogue containing one or more LNA nucleotide monomers with a bicyclic furanose unit locked in an RNA mimicking sugar conformation, which enhance hybridization affinity toward complementary RNA and DNA sequences (Vester and Wengel, 2004, Biochemistry 43(42):13233-41). RNA and DNA have different sugar moieties and can differ by the presence of uracil or analogs thereof in RNA and thymine or analogs thereof in DNA. [00263]“Guide RNA”, “gRNA”, and simply “guide” are used herein interchangeably to refer to, for example, the guide that directs an RNA-guided DNA binding agent to a target DNA and can be a single guide RNA, or the combination of a crRNA and a trRNA (also known as tracrRNA). Exemplary gRNAs include Class II Cas nuclease guide RNAs, in modified or unmodified forms. The crRNA and trRNA may be associated as a single RNA molecule (single guide RNA, sgRNA) or in two separate RNA strands (dual guide RNA, dgRNA). “Guide RNA” or “gRNA” refers to each type. The trRNA may be a naturally occurring sequence, or a trRNA sequence with modifications or variations compared to naturally-occurring sequences.

As used herein, a “guide sequence” refers to a sequence within a guide RNA that is complementary to a target sequence and functions to direct a guide RNA to a target sequence for binding or modification (e.g., cleavage) by an RNA-guided DNA binding agent. A “guide sequence” may also be referred to as a “targeting sequence,” or a “spacer sequence.” A guide sequence can be 19, 20, 21, 22, 23, or 24, or 25 nucleotides in length, e.g., in the case of Neisseria meningitides. In some embodiments, the Nme Cas9 guide sequence comprises at least 22, 23, or 24 contiguous nucleotides of a sequence selected from SEQ ID NOs: 2-80, 101-120, 201, 265, 301, 302, 304-576, or 601-774. In some embodiments, the target sequence is in a gene or on a chromosome, for example, and is complementary to the guide sequence. In some embodiments, the degree of complementarity or identity between a guide sequence and its corresponding target sequence is at least 80%, 85%, 90%, or 95%. For example, in some embodiments, the guide sequence comprises a sequence 24 contiguous nucleotides of a sequence selected from SEQ ID NO: 2-80, 101-120, 201, 265, 301, 302, 304-576, or 601-774. In some embodiments, the guide sequence and the target region may be 100% complementary or identical. In other embodiments, the guide sequence and the target region may contain at least one mismatch, i.e., one nucleotide that is not identical or not complementary, depending on the reference sequence. For example, the guide sequence and the target sequence may contain 1-2, preferably no more than 1 mismatch, where the total length of the target sequence is 19, 20, 21, 22, 23, or 24, nucleotides, or more. In some embodiments, the guide sequence and the target region may contain 1-2 mismatches where the guide sequence comprises at least 24 nucleotides, or more. In some embodiments, the guide sequence and the target region may contain 1-2 mismatches where the guide sequence comprises 24 nucleotides. That is, the guide sequence and the target region may form a duplex region having base pairs, or more. In certain embodiments, the duplex region may include 1-2 mismatches such that guide strand and target sequence are not fully complementary. Mismatch positions are known in the art as provided in, for example, PAM distal mismatches tend to be better tolerated than PAM proximal matches. Mismatch tolerances at other positions are known in the art (see, e.g., Edraki et al., 2019. Mol. Cell, 73:1-13).

Target sequences for RNA-guided DNA binding agents include both the positive and negative strands of genomic DNA (i.e., the sequence given and the sequence's reverse compliment), as a nucleic acid substrate for an RNA-guided DNA binding agent is a double stranded nucleic acid. Accordingly, where a guide sequence is said to be “complementary to a target sequence”, it is to be understood that the guide sequence may direct a guide RNA to bind to the reverse complement of a target sequence. Thus, in some embodiments, where the guide sequence binds the reverse complement of a target sequence, the guide sequence is identical to certain nucleotides of the target sequence (e.g., the target sequence not including the PAM) except for the substitution of U for T in the guide sequence.

As used herein, an “RNA-guided DNA binding agent” means a polypeptide or complex of polypeptides having RNA and DNA binding activity, or a DNA-binding subunit of such a complex, wherein the DNA binding activity is sequence-specific and depends on the presence of a PAM and the sequence of the guide RNA. Exemplary RNA-guided DNA binding agents include Cas cleavases/nickases and inactivated forms thereof (“dCas DNA binding agents”). “Cas nuclease”, also called “Cas protein” as used herein, encompasses Cas cleavases, Cas nickases, and dCas DNA binding agents. Cas cleavases/nickases and dCas DNA binding agents include a Csm or Cmr complex of a type III CRISPR system, the Cas10, Csm1, or Cmr2 subunit thereof, a Cascade complex of a type I CRISPR system, the Cas3 subunit thereof, and Class 2 Cas nucleases.

As used herein, a “Class 2 Cas nuclease” is a single-chain polypeptide with RNA-guided DNA binding activity. Class 2 Cas nucleases include Class 2 Cas cleavases/nickases (e.g., H840A, D10A, or N863A variants of Spy Cas9 and D16A and H588A of Nme Cas9, e.g., Nme2 Cas9), which further have RNA-guided DNA cleavases or nickase activity, and Class 2 dCas DNA binding agents, in which cleavase/nickase activity is inactivated. Class 2 Cas nucleases include, for example, Cas9, Cpf1, C2c1, C2c2, C2c3, HF Cas9 (e.g., N497A, R661A, Q695A, Q926A variants), HypaCas9 (e.g., N692A, M694A, Q695A, H698A variants), eSPCas9(1.0) (e.g., K810A, K1003A, R1060A variants), and eSPCas9(1.1) (e.g., K848A, K1003A, R1060A variants) proteins and modifications thereof. Cpf1 protein, Zetsche et al., Cell, 163: 1-13 (2015), is homologous to Cas9, and contains a RuvC-like nuclease domain. Cpf1 sequences of Zetsche are incorporated by reference in their entirety. See, e.g., Zetsche, Tables S1 and S3. See, e.g., Makarova et al., Nat Rev Microbiol, 13(11): 722-36 (2015); Shmakov et al., Molecular Cell, 60:385-397 (2015).

Several Cas9 orthologs have been obtained from N. meningitidis (Esvelt et al., NAT. METHODS, vol. 10, 2013, 1116-1121; Hou et al., PNAS, vol. 110, 2013, pages 15644-15649) (Nme1Cas9, Nme2Cas9, and Nme3Cas9). The Nme2Cas9 ortholog functions efficiently in mammalian cells, recognizes an N4CC PAM, and can be used for in vivo editing with cognate gRNAs (Ran et al., NATURE, vol. 520, 2015, pages 186-191; Kim et al., NAT. COMMUN., vol. 8, 2017, pages 14500). Nme2Cas9 can be specific and selective, e.g. capable of low off-target editing (Lee et al., MOL. THER., vol. 24, 2016, pages 645-654; Kim et al., 2017). See also e.g., WO/2020081568 (e.g., pages 28 and 42), describing an Nme2Cas9 D16A nickase, the contents of which are hereby incorporated by reference in its entirety. Throughout, “NmeCas9” or “Nme Cas9” is generic and encompasses any type of NmeCas9, including, Nme1Cas9, Nme2Cas9, and Nme3Cas9.

Exemplary nucleotide and polypeptide sequences of Cas9 molecules are provided in Table 7. Methods for identifying alternate nucleotide sequences encoding Cas9 polypeptide sequences, including alternate naturally occurring variants, are known in the art. Sequences with at least 75%, 80%, 85%, 90%, 95%, 96%, 97%, 98%, or 99% identity to any of the Cas9 nucleic acid sequences, or nucleic acid sequences encoding the amino acid sequences provided herein are also contemplated.

As used herein, the term “editor” refers to an agent comprising a polypeptide that is capable of making a modification within a DNA sequence. In some embodiments, the editor is a cleavase, such as a Cas9 cleavase. In some embodiments, the editor is capable of deaminating a base within a DNA molecule, and it may be called a base editor. In some embodiments, the editor is capable of deaminating a cytosine (C) in DNA. In some embodiments, the editor is a fusion protein comprising an RNA-guided nickase fused to a cytidine deaminase. In some embodiments, the editor is a fusion protein comprising an RNA-guided nickase fused to an APOBEC3A deaminase (A3A). In some embodiments, the editor comprises a Cas9 nickase fused to an APOBEC3A deaminase (A3A). In some embodiments, the editor is a fusion protein comprising an RNA-guided nickase fused to a cytidine deaminase and a UGI. In some embodiments, the editor lacks a UGI.

As used herein, a “cytidine deaminase” means a polypeptide or complex of polypeptides that is capable of cytidine deaminase activity, that is catalyzing the hydrolytic deamination of cytidine or deoxycytidine, typically resulting in uridine or deoxyuridine. Cytidine deaminases encompass enzymes in the cytidine deaminase superfamily, and in particular, enzymes of the APOBEC family (APOBEC1, APOBEC2, APOBEC4, and APOBEC3 subgroups of enzymes), activation-induced cytidine deaminase (AID or AICDA) and CMP deaminases (see, e.g., Conticello et al., Mol. Biol. Evol. 22:367-77, 2005; Conticello, Genome Biol. 9:229, 2008; Muramatsu et al., J. Biol. Chem. 274: 18470-6, 1999); Carrington et al., Cells 9:1690 (2020)). In some embodiments, variants of any known cytidine deaminase or APOBEC protein are encompassed. Variants include proteins having a sequence that differs from wild-type protein by one or several mutations (i.e., substitutions, deletions, insertions), such as one or several single point substitutions. For instance, a shortened sequence could be used, e.g., by deleting N-terminal, C-terminal, or internal amino acids, preferably one to four amino acids at the C-terminus of the sequence. As used herein, the term “variant” refers to allelic variants, splicing variants, and natural or artificial mutants, which are homologous to a reference sequence. The variant is “functional” in that it shows a catalytic activity of DNA editing.

As used herein, the term “APOBEC3A” refers to a cytidine deaminase such as the protein expressed by the human A3A gene. The APOBEC3A may have catalytic DNA editing activity. An amino acid sequence of APOBEC3A has been described (UniPROT accession ID: p31941) and is included herein as SEQ ID NO: 827. In some embodiments, the APOBEC3A protein is a human APOBEC3A protein or a wild-type protein. Variants include proteins having a sequence that differs from wild-type APOBEC3A protein by one or several mutations (i.e., substitutions, deletions, insertions), such as one or several single point substitutions. For instance, a shortened APOBEC3A sequence could be used, e.g., by deleting N-terminal, C-terminal, or internal amino acids, preferably one to four amino acids at the C-terminus of the sequence. As used herein, the term “variant” refers to allelic variants, splicing variants, and natural or artificial mutants, which are homologous to an APOBEC3A reference sequence. The variant is “functional” in that it shows a catalytic activity of DNA editing. In some embodiments, an APOBEC3A (such as a human APOBEC3A) has a wild-type amino acid position 57 (as numbered in the wild-type sequence). In some embodiments, an APOBEC3A (such as a human APOBEC3A) has an asparagine at amino acid position 57 (as numbered in the wild-type sequence).

As used herein, a “nickase” is an enzyme that creates a single-strand break (also known as a “nick”) in double strand DNA, i.e., cuts one strand but not the other of the DNA double helix. As used herein, an “RNA-guided DNA nickase” means a polypeptide or complex of polypeptides having DNA nickase activity, wherein the DNA nickase activity is sequence-specific and depends on the sequence of the RNA. Exemplary RNA-guided DNA nickases include Cas nickases. Class 2 Cas nickases include, polypeptides in which either the HNH or RuvC catalytic domain is inactivated, for example, Cas9 (e.g., H840A, D10A, or N863A variants of SpyCas9 or D16A variant of NmeCas9). Exemplary amino acid substitutions in the HNH or HNH-like nuclease domain or RuvC or RuvC-like domains for N. meningitidis include Nme2Cas9 D16A (HNH nickase) and Nme2Cas9 H588A (RuvC nickase). Cpf1, C2c1, C2c2, C2c3, HF Cas9 (e.g., N497A, R661A, Q695A, Q926A variants), HypaCas9 (e.g., N692A, M694A, Q695A, H698A variants), eSPCas9(1.0) (e.g., K810A, K1003A, R1060A variants), and eSPCas9(1.1) (e.g., K848A, K1003A, R1060A variants) proteins and modifications thereof. Cpf1 protein, Zetsche et al., Cell, 163: 1-13 (2015), is homologous to Cas9, and contains a RuvC-like protein domain. Cpf1 sequences of Zetsche are incorporated by reference in their entirety. See, e.g., Zetsche, Tables S1 and S3. “Cas9” encompasses S. pyogenes (Spy) Cas9, the variants of Cas9 listed herein, and equivalents thereof. See, e.g., Makarova et al., Nat Rev Microbiol, 13(11): 722-36 (2015); Shmakov et al., Molecular Cell, 60:385-397 (2015).

As used herein, the term “fusion protein” refers to a hybrid polypeptide which comprises protein domains from at least two different proteins. One protein may be located at the amino-terminal (N-terminal) portion of the fusion protein or at the carboxy-terminal (C-terminal) protein thus forming an “amino-terminal fusion protein” or a “carboxy-terminal fusion protein,” respectively. Any of the proteins provided herein may be produced by any method known in the art. For example, the proteins provided herein may be produced via recombinant protein expression and purification, which is especially suited for fusion proteins comprising a peptide linker. Methods for recombinant protein expression and purification are well known, and include those described by Green and Sambrook, Molecular Cloning: A Laboratory Manual (4th ed., Cold Spring Harbor Laboratory Press, Cold Spring Harbor, N.Y. (2012)), the entire contents of which are incorporated herein by reference.

The term “linker,” as used herein, refers to a chemical group or a molecule linking two adjacent molecules or moieties. Typically, the linker is positioned between, or flanked by, two groups, molecules, or other moieties and connected to each one via a covalent bond. In some embodiments, the linker is an amino acid or a plurality of amino acids (e.g., a peptide or protein) such as a 16-amino acid residue “XTEN” linker, or a variant thereof (See, e.g., the Examples; and Schellenberger et al. A recombinant polypeptide extends the in vivo half-life of peptides and proteins in a tunable manner. Nat. Biotechnol. 27, 1186-1190 (2009)). In some embodiments, the XTEN linker comprises the sequence SGSETPGTSESATPES (SEQ ID NO: 930), SGSETPGTSESA (SEQ ID NO: 931), or SGSETPGTSESATPEGGSGGS (SEQ ID NO: 932). In some embodiments, the linker is a peptide linker comprising one or more sequences selected from SEQ ID NOs: 933-994.

As used herein, the term “uracil glycosylase inhibitor” or “UGI” refers to a protein that is capable of inhibiting a uracil-DNA glycosylase (UDG) base-excision repair enzyme.

As used herein, “open reading frame” or “ORF” of a gene refers to a sequence consisting of a series of codons that specify the amino acid sequence of the protein that the gene codes for. The ORF begins with a start codon (e.g., ATG in DNA or AUG in RNA) and ends with a stop codon, e.g., TAA, TAG or TGA in DNA or UAA, UAG, or UGA in RNA.

As used herein, “ribonucleoprotein” (RNP) or “RNP complex” refers to a guide RNA together with an RNA-guided DNA binding agent, such as a Cas nuclease, e.g., a Cas cleavase, Cas nickase, or dCas DNA binding agent (e.g., Cas9). In some embodiments, the guide RNA guides the RNA-guided DNA binding agent such as Cas9 to a target sequence, and the guide RNA hybridizes with the target sequence and the agent binds to the target sequence; in cases where the agent is a cleavase or nickase, binding can be followed by cleaving or nicking.

As used herein, a first sequence is considered to “comprise a sequence with at least X % identity to” a second sequence if an alignment of the first sequence to the second sequence shows that X % or more of the positions of the second sequence in its entirety are matched by the first sequence. For example, the sequence AAGA comprises a sequence with 100% identity to the sequence AAG because an alignment would give 100% identity in that there are matches to all three positions of the second sequence. The differences between RNA and DNA (generally the exchange of uridine for thymidine or vice versa) and the presence of nucleoside analogs such as modified uridines do not contribute to differences in identity or complementarity among polynucleotides as long as the relevant nucleotides (such as thymidine, uridine, or modified uridine) have the same complement (e.g., adenosine for all of thymidine, uridine, or modified uridine; another example is cytosine and 5-methylcytosine, both of which have guanosine or modified guanosine as a complement). Thus, for example, the sequence 5′-AXG where X is any modified uridine, such as pseudouridine, N1-methyl pseudouridine, or 5-methoxyuridine, is considered 100% identical to AUG in that both are perfectly complementary to the same sequence (5′-CAU). Exemplary alignment algorithms are the Smith-Waterman and Needleman-Wunsch algorithms, which are well-known in the art. One skilled in the art will understand what choice of algorithm and parameter settings are appropriate for a given pair of sequences to be aligned; for sequences of generally similar length and expected identity >50% for amino acids or >75% for nucleotides, the Needleman-Wunsch algorithm with default settings of the Needleman-Wunsch algorithm interface provided by the EBI at the www.ebi.ac.uk web server is generally appropriate.

“Messenger RNA” or “mRNA” is used herein to refer to a polynucleotide and comprises an open reading frame that can be translated into a polypeptide (i.e., can serve as a substrate for translation by a ribosome and amino-acylated tRNAs). mRNA can comprise one or more modifications as provided below.

As used herein, a “genetic modification” is a change at the DNA level, e.g. induced by a CRISPR/Cas9 gRNA and Cas9 system. A genetic modification may comprise an insertion, deletion, or substitution (i.e., base sequence substitution, i.e., mutation), typically within a defined sequence or genomic locus. A genetic modification changes the nucleic acid sequence of the DNA. A genetic modification may be at a single nucleotide position. A genetic modification may be at multiple nucleotides, e.g., 2, 3, 4, 5 or more nucleotides, typically in close proximity to each other, e.g., contiguous nucleotides. A genetic modification can be in a coding sequence, e.g., an exon sequence. A genetic modification can be at a splice site, i.e., sufficiently close to a splice acceptor site or a splice donor site to disrupt splicing. A genetic modification can include insertion of a nucleotide sequence not endogenous to the genomic locus, e.g., insertion of a coding sequence of a heterologous open reading frame or gene. As used herein, a genetic modification can be used to prevent translation of an endogenous full-length protein having an amino acid sequence of the full-length protein prior to genetic modification of the genomic locus. Prevention of translation of an endogenous full-length protein or gene product includes prevention of translation of a protein or gene product of any length. Translation of an endogenous full-length protein can be prevented, for example, by a frameshift mutation that results in the generation of a premature stop codon or by generation of a nonsense mutation. Translation of an endogenous full-length protein can be prevented by disruption of splicing. Translation of an endogenous full-length protein can be prevented by the insertion of a heterologous coding sequence. Translation of an endogenous full-length protein, e.g., when the endogenous full-length protein contains an unwanted mutation, can be prevented by making a change at one or more positions to change an endogenous full-length protein coding sequence to provide a modified full-length coding sequence different from the endogenous sequence present in the cell, e.g., correction of a point mutation. Translation of an endogenous full-length protein can be prevented by altering the splicing of the endogenous full-length protein to produce a different protein by alternative splicing.

As used herein, “indel” refers to an insertion or deletion mutation consisting of a number of nucleotides that are either inserted, deleted, or inserted and deleted, e.g. at the site of double-stranded breaks (DSBs), in a target nucleic acid. As used herein, when indel formation results in an insertion, the insertion is a random insertion at the site of a DSB and may or may not be directed by or based on a template sequence.

As used herein, a “heterologous coding sequence” refers to a coding sequence that has been introduced as an exogenous source within a cell (e.g., inserted at a genomic locus such as a safe harbor locus including a TCR gene locus). That is, the introduced coding sequence is heterologous with respect to at least its insertion site. A polypeptide expressed from such heterologous coding sequence gene is referred to as a “heterologous polypeptide.” The heterologous coding sequence can be naturally-occurring or engineered, and can be wild-type or a variant. The heterologous coding sequence may include nucleotide sequences other than the sequence that encodes the heterologous polypeptide (e.g., an internal ribosomal entry site). The heterologous coding sequence can be a coding sequence that occurs naturally in the genome, as a wild-type or a variant (e.g., mutant). For example, although the cell contains the coding sequence of interest (as a wild-type or as a variant), the same coding sequence or variant thereof can be introduced as an exogenous source for, e.g., expression at a locus that is highly expressed. The heterologous gcoding sequence can also be a coding sequence that is not naturally occurring in the genome, or that expresses a heterologous polypeptide that does not naturally occur in the genome. “Heterologous coding sequence”, “exogenous coding sequence”, and “transgene” are used interchangeably. In some embodiments, the heterologous coding sequence or transgene includes an exogenous nucleic acid sequence, e.g., a nucleic acid sequence is not endogenous to the recipient cell. In some embodiments, the heterologous coding sequence or transgene includes an exogenous nucleic acid sequence, e.g., a nucleic acid sequence that does not naturally occur in the recipient cell. For example, a heterologous coding sequence may be heterologous with respect to its insertion site and with respect to its recipient cell.

As used herein, “reduced or eliminated” expression of a protein on a cell refers to a partial or complete loss of expression of the protein relative to an unmodified cell. In some embodiments, the surface expression of a protein on a cell is measured by flow cytometry and has “reduced” or “eliminated” surface expression relative to an unmodified cell as evidenced by a reduction in fluorescence signal upon staining with the same antibody against the protein. A cell that has “reduced” or “eliminated” surface expression of a protein by flow cytometry relative to an unmodified cell may be referred to as “negative” for expression of that protein as evidenced by a fluorescence signal similar to a cell stained with an isotype control antibody. The “reduction” or “elimination” of protein expression can be measured by other known techniques in the field with appropriate controls known to those skilled in the art.

As used herein, “knockdown” refers to a decrease in expression of a particular gene product (e.g., protein, mRNA, or both), e.g., as compared to expression of an unedited target sequence. Knockdown of a protein can be measured by detecting total cellular amount of the protein from a sample, such as a tissue, fluid, or cell population of interest. It can also be measured by measuring a surrogate, marker, or activity for the protein. Methods for measuring knockdown of mRNA are known and include analyzing mRNA isolated from a sample of interest. In some embodiments, “knockdown” may refer to some loss of expression of a particular gene product, for example a decrease in the amount of mRNA transcribed or a decrease in the amount of protein expressed by a cell or population of cells (including in vivo populations such as those found in tissues).

As used herein, “knockout” refers to a loss of expression from a particular gene or of a particular protein in a cell. Knockout can result in a decrease in expression below the level of detection of the assay. Knockout can be measured either by detecting total cellular amount of a protein in a cell, a tissue or a population of cells.

As used herein, a “target sequence” or “genomic target sequence” refers to a sequence of nucleic acid in a target gene that has complementarity to the guide sequence of the gRNA. The interaction of the target sequence and the guide sequence directs an RNA-guided DNA binding agent to bind, and potentially nick or cleave (depending on the activity of the agent), within the target sequence.

As used herein, “treatment” refers to any administration or application of a therapeutic for disease or disorder in a subject, and includes inhibiting the disease, arresting its development, relieving one or more symptoms of the disease, curing the disease, or preventing one or more symptoms of the disease, including recurrence of the symptom.

Reference will now be made in detail to certain embodiments of the invention, examples of which are illustrated in the accompanying drawings. While the invention is described in conjunction with the illustrated embodiments, it will be understood that they are not intended to limit the invention to those embodiments. On the contrary, the invention is intended to cover all alternatives, modifications, and equivalents, which may be included within the invention as defined by the appended claims and included embodiments.

Before describing the present teachings in detail, it is to be understood that the disclosure is not limited to specific compositions or process steps, as such may vary. It should be noted that, as used in this specification and the appended claims, the singular form “a”, “an” and “the” include plural references unless the context clearly dictates otherwise. Thus, for example, reference to “a conjugate” includes a plurality of conjugates and reference to “a cell” includes a plurality of cells and the like.

Numeric ranges are inclusive of the numbers defining the range. Measured and measurable values are understood to be approximate, taking into account significant digits and the error associated with the measurement. Also, the use of “comprise”, “comprises”, “comprising”, “contain”, “contains”, “containing”, “include”, “includes”, and “including” are not intended to be limiting. It is to be understood that both the foregoing general description and detailed description are exemplary and explanatory only and are not restrictive of the teachings.

Unless specifically noted in the specification, embodiments in the specification that recite “comprising” various components are also contemplated as “consisting of” or “consisting essentially of” the recited components; embodiments in the specification that recite “consisting of” various components are also contemplated as “comprising” or “consisting essentially of” the recited components; and embodiments in the specification that recite “consisting essentially of” various components are also contemplated as “consisting of” or “comprising” the recited components (this interchangeability does not apply to the use of these terms in the claims). The term “or” is used in an inclusive sense, i.e., equivalent to “and/or,” unless the context clearly indicates otherwise.

The section headings used herein are for organizational purposes only and are not to be construed as limiting the desired subject matter in any way. In the event that any material incorporated by reference contradicts any term defined in this specification or any other express content of this specification, this specification controls. While the present teachings are described in conjunction with various embodiments, it is not intended that the present teachings be limited to such embodiments. On the contrary, the present teachings encompass various alternatives, modifications, and equivalents, as will be appreciated by those of skill in the art.

### II. Genetically Modified Cells

### A. Engineered Cell Compositions

The present disclosure provides engineered cell compositions which have reduced or eliminated surface expression of HLA-A, HLA-B, TRAC, TRBC, and/or MHC class II relative to an unmodified cell as disclosed herein. In some embodiments, the engineered cell composition comprises a genetic modification in the HLA-A, HLA-B, TRAC, TRBC, and/or CIITA gene. In some embodiments, the engineered cell composition comprises a genetic modification in each of the HLA-A, HLA-B, and CIITA genes. In some embodiments, the engineered cell is an allogeneic cell. In some embodiments, the engineered cell with reduced HLA-A, HLA-B, TRAC, TRBC, and/or MHC class II expression is useful for adoptive cell transfer therapies. In some embodiments, the engineered cell comprises additional genetic modifications in the genome of the cell to yield a cell that is desirable for allogeneic transplant purposes.

In some embodiments, an engineered cell which has reduced or eliminated surface expression of HLA-A relative to an unmodified cell is provided, comprising a genetic modification in the HLA-A gene, wherein the modification comprises at least one nucleotide from within the genomic coordinates chr6:29942540-29945459. In some embodiments, an engineered cell which has reduced or eliminated surface expression of HLA-A relative to an unmodified cell is provided, comprising a genetic modification in the HLA-A gene, wherein the modification comprises at least one nucleotide from within the genomic coordinates chosen from: chr6:29942891-29942915; chr6:29942609-29942633; chr6:29944266-29944290; chr6:29942889-29942913; chr6:29944471-29944495; and chr6:29944470-29944494.

In some embodiments, an engineered cell which has reduced or eliminated surface expression of HLA-A relative to an unmodified cell is provided, comprising a genetic modification in the HLA-A gene, wherein the modification comprises at least one nucleotide from within the genomic coordinates chosen from: chr6:29942891-29942915; chr6:29942609-29942633; chr6:29942889-29942913; chr6:29944471-29944495; chr6:29944266-29944290; chr6:29942785-29942809.

In some embodiments, an engineered cell which has reduced or eliminated surface expression of HLA-A relative to an unmodified cell is provided, comprising a genetic modification in the HLA-A gene, wherein the modification comprises at least 5 contiguous nucleotides within the genomic coordinates chr6:29942540-29945459. In some embodiments, the engineered cell which has reduced or eliminated surface expression of HLA-A relative to an unmodified cell, comprises a genetic modification in the HLA-A gene, wherein the modification comprises at least 6, 7, 8, 9, or 10 contiguous nucleotides within the genomic coordinates.

In some embodiments, an engineered cell which has reduced or eliminated surface expression of HLA-A relative to an unmodified cell is provided, comprising a genetic modification in the HLA-A gene, wherein the modification comprises at least 5 contiguous nucleotides within the genomic coordinates chosen from: chr6:29942891-29942915; chr6:29942609-29942633; chr6:29944266-29944290; chr6:29942889-29942913; chr6:29944471-29944495; and chr6:29944470-29944494. In some embodiments, the engineered cell which has reduced or eliminated surface expression of HLA-A relative to an unmodified cell, comprises a genetic modification in the HLA-A gene, wherein the modification comprises at least 6, 7, 8, 9, or 10 contiguous nucleotides within the genomic coordinates.

In some embodiments, an engineered cell which has reduced or eliminated surface expression of HLA-A relative to an unmodified cell is provided, comprising a genetic modification in the HLA-A gene, wherein the modification comprises at least 5 contiguous nucleotides within the genomic coordinates chosen from: chr6:29942891-29942915; chr6:29942609-29942633; chr6:29942889-29942913; chr6:29944471-29944495; chr6:29944266-29944290; chr6:29942785-29942809. In some embodiments, the engineered cell which has reduced or eliminated surface expression of HLA-A relative to an unmodified cell, comprises a genetic modification in the HLA-A gene, wherein the modification comprises at least 6, 7, 8, 9, or 10 contiguous nucleotides within the genomic coordinates.

In some embodiments, an engineered cell which has reduced or eliminated surface expression of HLA-A relative to an unmodified cell is provided, comprising a genetic modification in the HLA-A gene, wherein the modification comprises at least one C to T substitution or at least one A to G substitution within the genomic coordinates chr6:29942540-29945459.

In some embodiments, an engineered cell which has reduced or eliminated surface expression of HLA-A relative to an unmodified cell is provided, comprising a genetic modification in the HLA-A gene, wherein the modification comprises at least one C to T substitution or at least one A to G substitution within the genomic coordinates chosen from: chr6:29942891-29942915; chr6:29942609-29942633; chr6:29944266-29944290; chr6:29942889-29942913; chr6:29944471-29944495; and chr6:29944470-29944494.

In some embodiments, an engineered cell which has reduced or eliminated surface expression of HLA-A relative to an unmodified cell is provided, comprising a genetic modification in the HLA-A gene, wherein the modification comprises at least one C to T substitution or at least one A to G substitution within the genomic coordinates chosen from: chr6:29942891-29942915; chr6:29942609-29942633; chr6:29942889-29942913; chr6:29944471-29944495; chr6:29944266-29944290; chr6:29942785-29942809.

In some embodiments, an engineered cell, which has reduced or eliminated surface expression of HLA-A relative to an unmodified cell, comprises a genetic modification in the HLA-A gene, wherein the genetic modification comprises an indel, a C to T substitution, or an A to G substitution within the genomic coordinates chosen from: (a) chr6:29942891-29942915; chr6:29942609-29942633; chr6:29944266-29944290; chr6:29942889-29942913; chr6:29944471-29944495; and chr6:29944470-29944494 or (b) chr6:29942891-29942915; chr6:29942609-29942633; chr6:29942889-29942913; chr6:29944471-29944495; chr6:29944266-29944290; chr6:29942785-29942809. In some embodiments, the genetic modification comprises at least 5 contiguous nucleotides within the genomic coordinates. In some embodiments, the genetic modification comprises at least 6, 7, 8, 9, or 10 contiguous nucleotides within the genomic coordinates. In some embodiments, the genetic modification comprises at least one C to T substitution or at least one A to G substitution within the genomic coordinates.

In some embodiments, an engineered cell is provided that has reduced or eliminated surface expression of HLA-A by a genomic editing system that binds to an HLA-A genomic target sequence comprising at least 10 contiguous nucleotides within the genomic coordinates chosen from: (a) chr6:29942891-29942915; chr6:29942609-29942633; chr6:29944266-29944290; chr6:29942889-29942913; chr6:29944471-29944495; and chr6:29944470-29944494 or (b) chr6:29942891-29942915; chr6:29942609-29942633; chr6:29942889-29942913; chr6:29944471-29944495; chr6:29944266-29944290; chr6:29942785-29942809. In some embodiments, an engineered cell is provided that has reduced or eliminated surface expression of HLA-A by a genomic editing system that binds to an HLA-A genomic target sequence comprising at least 5 contiguous nucleotides within the genomic coordinates. In some embodiments, the HLA-A genomic target sequence comprises at least 15 contiguous nucleotides within the genomic coordinates. In some embodiments, the genomic editing system comprises an RNA-guided DNA-binding agent. In some embodiments, the RNA-guided DNA-binding agent comprises a Cas9 protein, such as an N. meningitidis Cas9.

In some embodiments, an engineered cell is provided that has reduced or eliminated surface expression of HLA-A by a genomic editing system that binds to an HLA-A genomic target sequence comprising at least 5 contiguous nucleotides within the genomic coordinates chosen from: (a) chr6:29942891-29942915; chr6:29942609-29942633; chr6:29944266-29944290; chr6:29942889-29942913; chr6:29944471-29944495; and chr6:29944470-29944494 or (b) chr6:29942891-29942915; chr6:29942609-29942633; chr6:29942889-29942913; chr6:29944471-29944495; chr6:29944266-29944290; chr6:29942785-29942809. In some embodiments, the HLA-A genomic target sequence comprises at least 10 contiguous nucleotides within the genomic coordinates. In some embodiments, the HLA-A genomic target sequence comprises at least 15 contiguous nucleotides within the genomic coordinates. In some embodiments, the genomic editing system comprises an RNA-guided DNA-binding agent. In some embodiments, the RNA-guided DNA-binding agent comprises a Cas9 protein, such as an N. Meningitidis.

In some embodiments, an engineered cell which has reduced or eliminated surface expression of TRAC relative to an unmodified cell is provided, comprising a genetic modification in the TRAC gene, wherein the modification comprises at least one nucleotide from within the genomic coordinates chr14:22547462-22551621. In some embodiments, an engineered cell which has reduced or eliminated surface expression of TRAC relative to an unmodified cell is provided, comprising a genetic modification in the TRAC gene, wherein the modification comprises at least one nucleotide from within the genomic coordinates chr14:22547505-22551621. In some embodiments, an engineered cell which has reduced or eliminated surface expression of TRAC relative to an unmodified cell is provided, comprising a genetic modification in the TRAC gene, wherein the modification comprises at least one nucleotide from within the genomic coordinates from: chr14:22547505-22547529; chr14:22547525-22547549; chr14:22547674-22547698; chr14:22550544-22550568; or chr14:22550574-22550598.

In some embodiments, an engineered cell which has reduced or eliminated surface expression of TRAC relative to an unmodified cell is provided, comprising a genetic modification in the TRAC gene, wherein the modification comprises at least 5 contiguous nucleotides within the genomic coordinates chr14:22547462-22551621. In some embodiments, an engineered cell which has reduced or eliminated surface expression of TRAC relative to an unmodified cell is provided, comprising a genetic modification in the TRAC gene, wherein the modification comprises at least 5 contiguous nucleotides within the genomic coordinates chr14:22547505-22551621. In some embodiments, the engineered cell which has reduced or eliminated surface expression of TRAC relative to an unmodified cell, comprises a genetic modification in the TRAC gene, wherein the modification comprises at least 6, 7, 8, 9, or 10 contiguous nucleotides within the genomic coordinates.

In some embodiments, an engineered cell which has reduced or eliminated surface expression of TRAC relative to an unmodified cell is provided, comprising a genetic modification in the TRAC gene, wherein the modification comprises at least 5 contiguous nucleotides within the genomic coordinates chosen from: chr14:22547505-22547529; chr14:22547525-22547549; chr14:22547674-22547698; chr14:22550544-22550568; or chr14:22550574-22550598. In some embodiments, the engineered cell which has reduced or eliminated surface expression of TRAC relative to an unmodified cell, comprises a genetic modification in the TRAC gene, wherein the modification comprises at least 6, 7, 8, 9, or 10 contiguous nucleotides within the genomic coordinates.

In some embodiments, an engineered cell which has reduced or eliminated surface expression of TRAC relative to an unmodified cell is provided, comprising a genetic modification in the TRAC gene, wherein the modification comprises at least one C to T substitution or at least one A to G substitution within the genomic coordinates chr14:22547505-22551621 or chr14:22547462-22551621.

In some embodiments, an engineered cell which has reduced or eliminated surface expression of TRAC relative to an unmodified cell is provided, comprising a genetic modification in the TRAC gene, wherein the modification comprises at least one C to T substitution or at least one A to G substitution within the genomic coordinates chosen from: chr14:22547505-22547529; chr14:22547525-22547549; chr14:22547674-22547698; chr14:22550544-22550568; or chr14:22550574-22550598.

In some embodiments, an engineered cell, which has reduced or eliminated surface expression of TRAC relative to an unmodified cell, comprises a genetic modification in the TRAC gene, wherein the genetic modification comprises an indel, a C to T substitution, or an A to G substitution within the genomic coordinates chosen from: (a) chr14:22547505-22547529; chr14:22547525-22547549; chr14:22547674-22547698; chr14:22550544-22550568; and chr14:22550574-22550598. In some embodiments, the genetic modification comprises at least 5 contiguous nucleotides within the genomic coordinates. In some embodiments, the genetic modification comprises at least 6, 7, 8, 9, or 10 contiguous nucleotides within the genomic coordinates. In some embodiments, the genetic modification comprises at least one C to T substitution or at least one A to G substitution within the genomic coordinates.

In some embodiments, an engineered cell is provided that has reduced or eliminated surface expression of TRAC by a genomic editing system that binds to a TRAC genomic target sequence comprising at least 10 contiguous nucleotides within the genomic coordinates chosen from: (a) chr14:22547505-22547529; chr14:22547525-22547549; chr14:22547674-22547698; chr14:22550544-22550568; and chr14:22550574-22550598. In some embodiments, an engineered cell is provided that has reduced or eliminated surface expression of TRAC by a genomic editing system that binds to a TRAC genomic target sequence comprising at least 5 contiguous nucleotides within the genomic coordinates. In some embodiments, the TRAC genomic target sequence comprises at least 15 contiguous nucleotides within the genomic coordinates. In some embodiments, the genomic editing system comprises an RNA-guided DNA-binding agent. In some embodiments, the RNA-guided DNA-binding agent comprises a Cas9 protein, such as an N. meningitidis Cas9.

In some embodiments, an engineered cell is provided that has reduced or eliminated surface expression of TRAC by a genomic editing system that binds to a TRAC genomic target sequence comprising at least 5 contiguous nucleotides within the genomic coordinates chosen from: (a) chr14:22547505-22547529; chr14:22547525-22547549; chr14:22547674-22547698; chr14:22550544-22550568; and chr14:22550574-22550598. In some embodiments, the TRAC genomic target sequence comprises at least 10 contiguous nucleotides within the genomic coordinates. In some embodiments, the TRAC genomic target sequence comprises at least 15 contiguous nucleotides within the genomic coordinates. In some embodiments, the genomic editing system comprises an RNA-guided DNA-binding agent. In some embodiments, the RNA-guided DNA-binding agent comprises a Cas9 protein, such as an N. Meningitidis.

In some embodiments, an engineered cell which has reduced or eliminated surface expression of TRBC relative to an unmodified cell is provided, comprising a genetic modification in the TRBC gene, wherein the modification comprises at least one nucleotide from within the genomic coordinates chosen from: chr7:142791756-142802543. In some embodiments, an engineered cell which has reduced or eliminated surface expression of TRBC relative to an unmodified cell is provided, comprising a genetic modification in the TRBC gene, wherein the modification comprises at least one nucleotide from within the genomic coordinates chosen from: (a) chr7:142791862-142793149; (b) chr7: 142791756-142792721; or (c) chr7:142801104-142802543.

In some embodiments, an engineered cell which has reduced or eliminated surface expression of TRBC relative to an unmodified cell is provided, comprising a genetic modification in the TRBC gene, wherein the modification comprises at least one nucleotide from within the genomic coordinates from: (a) chr7:142792690-142792714; or chr7:142792693-142792717; or (b) chr7:142791756-142791780; chr7:142791761-142791785; chr7:142791820-142791844; chr7:142791939-142791963; chr7:142791940-142791964; or chr7:142792004-142792028; or (c) chr7:142801104-142801124; chr7:142802103-142802127; or chr7:142802106-142802130.

In some embodiments, an engineered cell which has reduced or eliminated surface expression of TRBC relative to an unmodified cell is provided, comprising a genetic modification in the TRBC gene, wherein the modification comprises at least one nucleotide from within the genomic coordinates chosen from: chr7:142792690-142792714; chr7:142802103-142802127; and chr7:142802106-14280213.

In some embodiments, an engineered cell which has reduced or eliminated surface expression of TRBC relative to an unmodified cell is provided, comprising a genetic modification in the TRBC gene, wherein the modification comprises at least 5 contiguous nucleotides within the genomic coordinates chosen from: (a) chr7:142791862-142793149; (b) chr7: 142791756-142792721; or (c) chr7:142801104-142802543. In some embodiments, the engineered cell which has reduced or eliminated surface expression of TRBC relative to an unmodified cell, comprises a genetic modification in the TRBC gene, wherein the modification comprises at least 6, 7, 8, 9, or 10 contiguous nucleotides within the genomic coordinates.

In some embodiments, an engineered cell which has reduced or eliminated surface expression of TRBC relative to an unmodified cell is provided, comprising a genetic modification in the TRBC gene, wherein the modification comprises at least 5 contiguous nucleotides within the genomic coordinates chosen from: (a) chr7:142792690-142792714; or chr7:142792693-142792717; or (b) chr7:142791756-142791780; chr7:142791761-142791785; chr7:142791820-142791844; chr7:142791939-142791963; chr7:142791940-142791964; or chr7:142792004-142792028; or (c) chr7:142801104-142801124; chr7:142802103-142802127; or chr7:142802106-142802130. In some embodiments, the engineered cell which has reduced or eliminated surface expression of TRBC relative to an unmodified cell, comprises a genetic modification in the TRBC gene, wherein the modification comprises at least 6, 7, 8, 9, or 10 contiguous nucleotides within the genomic coordinates.

In some embodiments, an engineered cell which has reduced or eliminated surface expression of TRBC relative to an unmodified cell is provided, comprising a genetic modification in the TRBC gene, wherein the modification comprises at least 5 contiguous nucleotides within the genomic coordinates chosen from: chr7:142792690-142792714; chr7:142802103-142802127; and chr7:142802106-14280213. In some embodiments, the engineered cell which has reduced or eliminated surface expression of TRBC relative to an unmodified cell, comprises a genetic modification in the TRBC gene, wherein the modification comprises at least 6, 7, 8, 9, or 10 contiguous nucleotides within the genomic coordinates.

In some embodiments, an engineered cell which has reduced or eliminated surface expression of TRBC relative to an unmodified cell is provided, comprising a genetic modification in the TRBC gene, wherein the modification comprises at least one C to T substitution or at least one A to G substitution within the genomic coordinates chosen from:

In some embodiments, an engineered cell which has reduced or eliminated surface expression of TRBC relative to an unmodified cell is provided, comprising a genetic modification in the TRBC gene, wherein the modification comprises at least one C to T substitution or at least one A to G substitution within the genomic coordinates chosen from: (a) chr7:142791862-142793149; (b) chr7: 142791756-142792721; or (c) chr7:142801104-142802543.

In some embodiments, an engineered cell which has reduced or eliminated surface expression of TRBC relative to an unmodified cell is provided, comprising a genetic modification in the TRBC gene, wherein the modification comprises at least one C to T substitution or at least one A to G substitution within the genomic coordinates chosen from: (a) chr7:142792690-142792714; or chr7:142792693-142792717; or (b) chr7:142791756-142791780; chr7:142791761-142791785; chr7:142791820-142791844; chr7:142791939-142791963; chr7:142791940-142791964; or chr7:142792004-142792028; or (c) chr7:142801104-142801124; chr7:142802103-142802127; or chr7:142802106-142802130.

In some embodiments, an engineered cell which has reduced or eliminated surface expression of TRBC relative to an unmodified cell is provided, comprising a genetic modification in the TRBC gene, wherein the modification comprises at least one C to T substitution or at least one A to G substitution within the genomic coordinates chosen from: chr7:142792690-142792714; chr7:142802103-142802127; and chr7:142802106-14280213.

In some embodiments, an engineered cell, which has reduced or eliminated surface expression of TRBC relative to an unmodified cell, comprises a genetic modification in the TRBC gene, wherein the genetic modification comprises an indel, a C to T substitution, or an A to G substitution within the genomic coordinates chosen from: (a) chr7:142792690-142792714; or chr7:142792693-142792717; or (b) chr7:142791756-142791780; chr7:142791761-142791785; chr7:142791820-142791844; chr7:142791939-142791963; chr7:142791940-142791964; or chr7:142792004-142792028; or (c) chr7:142801104-142801124; chr7:142802103-142802127; or chr7:142802106-142802130. In some embodiments, the genetic modification comprises at least 5 contiguous nucleotides within the genomic coordinates. In some embodiments, the genetic modification comprises at least 6, 7, 8, 9, or 10 contiguous nucleotides within the genomic coordinates. In some embodiments, the genetic modification comprises at least one C to T substitution or at least one A to G substitution within the genomic coordinates.

In some embodiments, an engineered cell, which has reduced or eliminated surface expression of TRBC relative to an unmodified cell, comprises a genetic modification in the TRBC gene, wherein the genetic modification comprises an indel, a C to T substitution, or an A to G substitution within the genomic coordinates chosen from: chr7:142792690-142792714; chr7:142802103-142802127; and chr7:142802106-14280213. In some embodiments, the genetic modification comprises at least 5 contiguous nucleotides within the genomic coordinates. In some embodiments, the genetic modification comprises at least 6, 7, 8, 9, or 10 contiguous nucleotides within the genomic coordinates. In some embodiments, the genetic modification comprises at least one C to T substitution or at least one A to G substitution within the genomic coordinates.

In some embodiments, an engineered cell is provided that has reduced or eliminated surface expression of TRBC by a genomic editing system that binds to a TRBC genomic target sequence comprising at least 10 contiguous nucleotides within the genomic coordinates chosen from: (a) chr7:142792690-142792714; or chr7:142792693-142792717; or (b) chr7:142791756-142791780; chr7:142791761-142791785; chr7:142791820-142791844; chr7:142791939-142791963; chr7:142791940-142791964; or chr7:142792004-142792028; or (c) chr7:142801104-142801124; chr7:142802103-142802127; or chr7:142802106-142802130. In some embodiments, an engineered cell is provided that has reduced or eliminated surface expression of TRBC by a genomic editing system that binds to a TRBC genomic target sequence comprising at least 5 contiguous nucleotides within the genomic coordinates. In some embodiments, the TRBC genomic target sequence comprises at least 15 contiguous nucleotides within the genomic coordinates. In some embodiments, the genomic editing system comprises an RNA-guided DNA-binding agent. In some embodiments, the RNA-guided DNA-binding agent comprises a Cas9 protein, such as an N. meningitidis Cas9.

In some embodiments, an engineered cell is provided that has reduced or eliminated surface expression of TRBC by a genomic editing system that binds to a TRBC genomic target sequence comprising at least 10 contiguous nucleotides within the genomic coordinates chosen from: chr7:142792690-142792714; chr7:142802103-142802127; and chr7:142802106-14280213. In some embodiments, an engineered cell is provided that has reduced or eliminated surface expression of TRBC by a genomic editing system that binds to a TRBC genomic target sequence comprising at least 5 contiguous nucleotides within the genomic coordinates. In some embodiments, the TRBC genomic target sequence comprises at least 15 contiguous nucleotides within the genomic coordinates. In some embodiments, the genomic editing system comprises an RNA-guided DNA-binding agent. In some embodiments, the RNA-guided DNA-binding agent comprises a Cas9 protein, such as an N. meningitidis Cas9.

In some embodiments, an engineered cell is provided that has reduced or eliminated surface expression of TRBC by a genomic editing system that binds to a TRBC genomic target sequence comprising at least 5 contiguous nucleotides within the genomic coordinates chosen from: (a) chr7:142792690-142792714; or chr7:142792693-142792717; or (b) chr7:142791756-142791780; chr7:142791761-142791785; chr7:142791820-142791844; chr7:142791939-142791963; chr7:142791940-142791964; or chr7:142792004-142792028; or (c) chr7:142801104-142801124; chr7:142802103-142802127; or chr7:142802106-142802130. In some embodiments, the TRBC genomic target sequence comprises at least 10 contiguous nucleotides within the genomic coordinates. In some embodiments, the TRBC genomic target sequence comprises at least 15 contiguous nucleotides within the genomic coordinates. In some embodiments, the genomic editing system comprises an RNA-guided DNA-binding agent. In some embodiments, the RNA-guided DNA-binding agent comprises a Cas9 protein, such as an N. Meningitidis.

In some embodiments, an engineered cell is provided that has reduced or eliminated surface expression of TRBC by a genomic editing system that binds to a TRBC genomic target sequence comprising at least 5 contiguous nucleotides within the genomic coordinates chosen from: chr7:142792690-142792714; chr7:142802103-142802127; and chr7:142802106-14280213. In some embodiments, the TRBC genomic target sequence comprises at least 10 contiguous nucleotides within the genomic coordinates. In some embodiments, the TRBC genomic target sequence comprises at least 15 contiguous nucleotides within the genomic coordinates. In some embodiments, the genomic editing system comprises an RNA-guided DNA-binding agent. In some embodiments, the RNA-guided DNA-binding agent comprises a Cas9 protein, such as an N. Meningitidis.

In some embodiments, an engineered cell which has reduced or eliminated surface expression of MHC class II relative to an unmodified cell is provided, comprising a genetic modification in the CIITA locus, wherein the modification comprises at least one nucleotide of the genomic coordinates (a) chr16:10877363-10907788 or (b) chr16:10906515-10908136.

In some embodiments, an engineered cell which has reduced or eliminated surface expression of MHC class II relative to an unmodified cell is provided, comprising a genetic modification in the CIITA gene, wherein the modification comprises at least one nucleotide of the genomic coordinates chosen from: (a) chr16:10907504-10907528; chr16:10907508-10907532; chr16:10907539-10907559; chr16:10895658-10895682; chr16:10895668-10895692; chr16:10895750-10895774; chr16:10895753-10895777; chr16:10895754-10895778; chr16:10898684-10898708; chr16:10901529-10901553; chr16:10902121-10902145; chr16:10902701-10902725; chr16:10904726-10904750; chr16:10904760-10904784; chr16:10906493-10906517; chr16:10906515-10906539; chr16:10906631-10906655; chr16:10906636-10906660; chr16:10906643-10906667; chr16:10906770-10906794; chr16:10906788-10906812; chr16:10906789-10906813; chr16:10906816-10906840; chr16:10907148-10907172; chr16:10907254-10907278; chr16:10907331-10907355; chr16:10907477-10907501; chr16:10907497-10907521; chr16:10907503-10907527; and chr16:10907574-10907598; or (b) chr16:10906889-10906913; and chr16:10907504-10907528.

In some embodiments, an engineered cell which has reduced or eliminated surface expression of MHC class II relative to an unmodified cell is provided, comprising a genetic modification in the CIITA gene, wherein the modification comprises at least one nucleotide of the genomic coordinates chosen from: chr16:10895658-10895682; chr16:10902701-10902725; chr16:10906493-10906517; chr16:10906631-10906655; chr16:10906643-10906667; chr16:10907477-10907501; and chr16:10907497-10907521; chr16:10907504-10907528; and chr16:10907508-10907532.

In some embodiments, an engineered cell which has reduced or eliminated surface expression of MHC class II relative to an unmodified cell is provided, comprising a genetic modification in the CIITA gene, wherein the modification comprises at least 5 contiguous nucleotides within the genomic coordinates chosen from: (a) chr16:10877363-10907788 or (b) chr16:10906515-10908136. In some embodiments, the engineered cell which has reduced or eliminated surface expression of MHC class II relative to an unmodified cell, comprises a genetic modification in the CIITA gene, wherein the modification comprises at least 6, 7, 8, 9, or 10 contiguous nucleotides within the genomic coordinates.

In some embodiments, an engineered cell which has reduced or eliminated surface expression of MHC class II relative to an unmodified cell is provided, comprising a genetic modification in the CIITA gene, wherein the modification comprises at least 5 contiguous nucleotides within the genomic coordinates chosen from: (a) chr16:10907504-10907528; chr16:10907508-10907532; chr16:10907539-10907559; chr16:10895658-10895682; chr16:10895668-10895692; chr16:10895750-10895774; chr16:10895753-10895777; chr16:10895754-10895778; chr16:10898684-10898708; chr16:10901529-10901553; chr16:10902121-10902145; chr16:10902701-10902725; chr16:10904726-10904750; chr16:10904760-10904784; chr16:10906493-10906517; chr16:10906515-10906539; chr16:10906631-10906655; chr16:10906636-10906660; chr16:10906643-10906667; chr16:10906770-10906794; chr16:10906788-10906812; chr16:10906789-10906813; chr16:10906816-10906840; chr16:10907148-10907172; chr16:10907254-10907278; chr16:10907331-10907355; chr16:10907477-10907501; chr16:10907497-10907521; chr16:10907503-10907527; chr16:10907574-10907598; or (b) chr16:10906889-10906913; or chr16:10907504-10907528. In some embodiments, the engineered cell which has reduced or eliminated surface expression of MHC class II relative to an unmodified cell, comprises a genetic modification in the CIITA gene, wherein the modification comprises at least 6, 7, 8, 9, or 10 contiguous nucleotides within the genomic coordinates.

In some embodiments, an engineered cell which has reduced or eliminated surface expression of MHC class II relative to an unmodified cell is provided, comprising a genetic modification in the CIITA gene, wherein the modification comprises at least 5 contiguous nucleotides within the genomic coordinates chosen from: chr16:10895658-10895682; chr16:10902701-10902725; chr16:10906493-10906517; chr16:10906631-10906655; chr16:10906643-10906667; chr16:10907477-10907501; and chr16:10907497-10907521; chr16:10907504-10907528; and chr16:10907508-10907532. In some embodiments, the engineered cell which has reduced or eliminated surface expression of MHC class II relative to an unmodified cell, comprises a genetic modification in the CIITA gene, wherein the modification comprises at least 6, 7, 8, 9, or 10 contiguous nucleotides within the genomic coordinates.

In some embodiments, an engineered cell which has reduced or eliminated surface expression of MHC class II relative to an unmodified cell is provided, comprising a genetic modification in the CIITA gene, wherein the modification comprises at least one C to T substitution or at least one A to G substitution within the genomic coordinates chosen from: (a) chr16:10877363-10907788 or (b) chr16:10906515-10908136.

In some embodiments, an engineered cell which has reduced or eliminated surface expression of MHC class II relative to an unmodified cell is provided, comprising a genetic modification in the CIITA gene, wherein the modification comprises at least one C to T substitution or at least one A to G substitution within the genomic coordinates chosen from: (a) chr16:10907504-10907528; chr16:10907508-10907532; chr16:10907539-10907559; chr16:10895658-10895682; chr16:10895668-10895692; chr16:10895750-10895774; chr16:10895753-10895777; chr16:10895754-10895778; chr16:10898684-10898708; chr16:10901529-10901553; chr16:10902121-10902145; chr16:10902701-10902725; chr16:10904726-10904750; chr16:10904760-10904784; chr16:10906493-10906517; chr16:10906515-10906539; chr16:10906631-10906655; chr16:10906636-10906660; chr16:10906643-10906667; chr16:10906770-10906794; chr16:10906788-10906812; chr16:10906789-10906813; chr16:10906816-10906840; chr16:10907148-10907172; chr16:10907254-10907278; chr16:10907331-10907355; chr16:10907477-10907501; chr16:10907497-10907521; chr16:10907503-10907527; chr16:10907574-10907598; or (b) chr16:10906889-10906913; or chr16:10907504-10907528.

In some embodiments, an engineered cell which has reduced or eliminated surface expression of MHC class II relative to an unmodified cell is provided, comprising a genetic modification in the CIITA gene, wherein the modification comprises at least one C to T substitution or at least one A to G substitution within the genomic coordinates chosen from: chr16:10895658-10895682; chr16:10902701-10902725; chr16:10906493-10906517; chr16:10906631-10906655; chr16:10906643-10906667; chr16:10907477-10907501; and chr16:10907497-10907521; chr16:10907504-10907528; and chr16:10907508-10907532.

In some embodiments, an engineered cell which has reduced or eliminated surface expression of MHC class II relative to an unmodified cell is provided, comprising a genetic modification in the CIITA gene, wherein the modification comprises at least one nucleotide within the genomic coordinates chosen from: (a) chr16:10877363-10907788 or (b) chr16:10906515-10908136.

In some embodiments, an engineered cell which has reduced or eliminated surface expression of MHC class II relative to an unmodified cell is provided, comprising a genetic modification in the CIITA gene, wherein the modification comprises at least one nucleotide within the genomic coordinates chosen from: (a) chr16:10907504-10907528; chr16:10907508-10907532; chr16:10907539-10907559; chr16:10895658-10895682; chr16:10895668-10895692; chr16:10895750-10895774; chr16:10895753-10895777; chr16:10895754-10895778; chr16:10898684-10898708; chr16:10901529-10901553; chr16:10902121-10902145; chr16:10902701-10902725; chr16:10904726-10904750; chr16:10904760-10904784; chr16:10906493-10906517; chr16:10906515-10906539; chr16:10906631-10906655; chr16:10906636-10906660; chr16:10906643-10906667; chr16:10906770-10906794; chr16:10906788-10906812; chr16:10906789-10906813; chr16:10906816-10906840; chr16:10907148-10907172; chr16:10907254-10907278; chr16:10907331-10907355; chr16:10907477-10907501; chr16:10907497-10907521; chr16:10907503-10907527; chr16:10907574-10907598; or (b) chr16:10906889-10906913; or chr16:10907504-10907528.

In some embodiments, an engineered cell which has reduced or eliminated surface expression of MHC class II relative to an unmodified cell is provided, comprising a genetic modification in the CIITA gene, wherein the modification comprises at least one nucleotide within the genomic coordinates chosen from: chr16:10895658-10895682; chr16:10902701-10902725; chr16:10906493-10906517; chr16:10906631-10906655; chr16:10906643-10906667; chr16:10907477-10907501; and chr16:10907497-10907521; chr16:10907504-10907528; and chr16:10907508-10907532.

In some embodiments, an engineered cell, which has reduced or eliminated surface expression of MHC class II relative to an unmodified cell, comprises a genetic modification in the CIITA gene, the genetic modification comprises at least one nucleotide within the genomic coordinates chosen from: (a) chr16:10877363-10907788 or (b) chr16:10906515-10908136.

In some embodiments, an engineered cell, which has reduced or eliminated surface expression of MHC class II relative to an unmodified cell, comprises a genetic modification in the CIITA gene, the genetic modification comprises at least one nucleotide within the genomic coordinates chosen from: chr16:10907504-10907528; chr16:10907508-10907532; chr16:10907539-10907559; chr16:10895658-10895682; chr16:10895668-10895692; chr16:10895750-10895774; chr16:10895753-10895777; chr16:10895754-10895778; chr16:10898684-10898708; chr16:10901529-10901553; chr16:10902121-10902145; chr16:10902701-10902725; chr16:10904726-10904750; chr16:10904760-10904784; chr16:10906493-10906517; chr16:10906515-10906539; chr16:10906631-10906655; chr16:10906636-10906660; chr16:10906643-10906667; chr16:10906770-10906794; chr16:10906788-10906812; chr16:10906789-10906813; chr16:10906816-10906840; chr16:10907148-10907172; chr16:10907254-10907278; chr16:10907331-10907355; chr16:10907477-10907501; chr16:10907497-10907521; chr16:10907503-10907527; chr16:10907574-10907598; or (b) chr16:10906889-10906913; or chr16:10907504-10907528.

In some embodiments, an engineered cell, which has reduced or eliminated surface expression of MHC class II relative to an unmodified cell, comprises a genetic modification in the CIITA gene, the genetic modification comprises at least one nucleotide within the genomic coordinates chosen from: chr16:10895658-10895682; chr16:10902701-10902725; chr16:10906493-10906517; chr16:10906631-10906655; chr16:10906643-10906667; chr16:10907477-10907501; and chr16:10907497-10907521; chr16:10907504-10907528; and chr16:10907508-10907532.

In some embodiments, an engineered cell, which has reduced or eliminated surface expression of MHC class II relative to an unmodified cell, comprises a genetic modification in the CIITA gene, wherein the genetic modification comprises an indel, a C to T substitution, or an A to G substitution within the genomic coordinates chosen from: (a) chr16:10907504-10907528; chr16:10907508-10907532; chr16:10907539-10907559; chr16:10895658-10895682; chr16:10895668-10895692; chr16:10895750-10895774; chr16:10895753-10895777; chr16:10895754-10895778; chr16:10898684-10898708; chr16:10901529-10901553; chr16:10902121-10902145; chr16:10902701-10902725; chr16:10904726-10904750; chr16:10904760-10904784; chr16:10906493-10906517; chr16:10906515-10906539; chr16:10906631-10906655; chr16:10906636-10906660; chr16:10906643-10906667; chr16:10906770-10906794; chr16:10906788-10906812; chr16:10906789-10906813; chr16:10906816-10906840; chr16:10907148-10907172; chr16:10907254-10907278; chr16:10907331-10907355; chr16:10907477-10907501; chr16:10907497-10907521; chr16:10907503-10907527; chr16:10907574-10907598; or (b) chr16:10906889-10906913; or chr16:10907504-10907528. In some embodiments, the genetic modification comprises at least 5 contiguous nucleotides within the genomic coordinates. In some embodiments, the genetic modification comprises at least 6, 7, 8, 9, or 10 contiguous nucleotides within the genomic coordinates. In some embodiments, the genetic modification comprises at least one C to T substitution or at least one A to G substitution within the genomic coordinates.

In some embodiments, an engineered cell, which has reduced or eliminated surface expression of MHC class II relative to an unmodified cell, comprises a genetic modification in the CIITA gene, wherein the genetic modification comprises an indel, a C to T substitution, or an A to G substitution within the genomic coordinates chosen from: chr16:10895658-10895682; chr16:10902701-10902725; chr16:10906493-10906517; chr16:10906631-10906655; chr16:10906643-10906667; chr16:10907477-10907501; and chr16:10907497-10907521; chr16:10907504-10907528; and chr16:10907508-10907532. In some embodiments, the genetic modification comprises at least 5 contiguous nucleotides within the genomic coordinates. In some embodiments, the genetic modification comprises at least 6, 7, 8, 9, or 10 contiguous nucleotides within the genomic coordinates. In some embodiments, the genetic modification comprises at least one C to T substitution or at least one A to G substitution within the genomic coordinates.

In some embodiments, an engineered cell is provided that has reduced or eliminated surface expression of MHC class II by a genomic editing system that binds to a CIITA genomic target sequence comprising at least 10 contiguous nucleotides within the genomic coordinates chosen from: (a) chr16:10907504-10907528; chr16:10907508-10907532; chr16:10907539-10907559; chr16:10895658-10895682; chr16:10895668-10895692; chr16:10895750-10895774; chr16:10895753-10895777; chr16:10895754-10895778; chr16:10898684-10898708; chr16:10901529-10901553; chr16:10902121-10902145; chr16:10902701-10902725; chr16:10904726-10904750; chr16:10904760-10904784; chr16:10906493-10906517; chr16:10906515-10906539; chr16:10906631-10906655; chr16:10906636-10906660; chr16:10906643-10906667; chr16:10906770-10906794; chr16:10906788-10906812; chr16:10906789-10906813; chr16:10906816-10906840; chr16:10907148-10907172; chr16:10907254-10907278; chr16:10907331-10907355; chr16:10907477-10907501; chr16:10907497-10907521; chr16:10907503-10907527; chr16:10907574-10907598; or (b) chr16:10906889-10906913; or chr16:10907504-10907528.

In some embodiments, an engineered cell is provided that has reduced or eliminated surface expression of MHC class II by a genomic editing system that binds to a CIITA genomic target sequence comprising at least 10 contiguous nucleotides within the genomic coordinates chosen from: (a) chr16:10895658-10895682; chr16:10902701-10902725; chr16:10906493-10906517; chr16:10906631-10906655; chr16:10906643-10906667; chr16:10907477-10907501; and chr16:10907497-10907521; chr16:10907504-10907528; and chr16:10907508-10907532. In some embodiments, an engineered cell is provided that has reduced or eliminated surface expression of MHC class II by a genomic editing system that binds to a CIITA genomic target sequence comprising at least 5 contiguous nucleotides within the genomic coordinates. In some embodiments, the CIITA genomic target sequence comprises at least 15 contiguous nucleotides within the genomic coordinates. In some embodiments, the genomic editing system comprises an RNA-guided DNA-binding agent. In some embodiments, the RNA-guided DNA-binding agent comprises a Cas9 protein, such as an N. meningitidis Cas9.

In some embodiments, an engineered cell is provided that has reduced or eliminated surface expression of MHC class II by a genomic editing system that binds to a CIITA genomic target sequence comprising at least 5 contiguous nucleotides within the genomic coordinates chosen from: (a) chr16:10907504-10907528; chr16:10907508-10907532; chr16:10907539-10907559; chr16:10895658-10895682; chr16:10895668-10895692; chr16:10895750-10895774; chr16:10895753-10895777; chr16:10895754-10895778; chr16:10898684-10898708; chr16:10901529-10901553; chr16:10902121-10902145; chr16:10902701-10902725; chr16:10904726-10904750; chr16:10904760-10904784; chr16:10906493-10906517; chr16:10906515-10906539; chr16:10906631-10906655; chr16:10906636-10906660; chr16:10906643-10906667; chr16:10906770-10906794; chr16:10906788-10906812; chr16:10906789-10906813; chr16:10906816-10906840; chr16:10907148-10907172; chr16:10907254-10907278; chr16:10907331-10907355; chr16:10907477-10907501; chr16:10907497-10907521; chr16:10907503-10907527; chr16:10907574-10907598; or (b) chr16:10906889-10906913; or chr16:10907504-10907528.

In some embodiments, an engineered cell is provided that has reduced or eliminated surface expression of MHC class II by a genomic editing system that binds to a CIITA genomic target sequence comprising at least 5 contiguous nucleotides within the genomic coordinates chosen from: chr16:10895658-10895682; chr16:10902701-10902725; chr16:10906493-10906517; chr16:10906631-10906655; chr16:10906643-10906667; chr16:10907477-10907501; and chr16:10907497-10907521; chr16:10907504-10907528; and chr16:10907508-10907532 . . . In some embodiments, the CIITA genomic target sequence comprises at least 10 contiguous nucleotides within the genomic coordinates. In some embodiments, the CIITA genomic target sequence comprises at least 15 contiguous nucleotides within the genomic coordinates. In some embodiments, the genomic editing system comprises an RNA-guided DNA-binding agent. In some embodiments, the RNA-guided DNA-binding agent comprises a Cas9 protein, such as an N. Meningitidis.

In some embodiments, an engineered cell is provided comprising a genetic modification in the AAVS1 gene, wherein the modification comprises at least one nucleotide from within the genomic coordinates chr19:55115151-55116209.

In some embodiments, an engineered cell is provided, comprising a genetic modification in the AAVS1 gene, wherein the modification comprises at least one nucleotide from within the genomic coordinates chosen from: chr19:55115218-55115242; chr19:55115477-55115501; chr19:55115504-55115528; chr19:55115513-55115537; chr19:55115514-55115538; chr19:55115517-55115541; chr19:55115518-55115542; chr19:55115549-55115573; chr19:55115574-55115598; chr19:55115606-55115630; chr19:55115933-55115957; chr19:55116026-55116050; chr19:55116045-55116069; chr19:55116084-55116108; chr19:55115276-55115300; chr19:55115509-55115533; chr19:55115579-55115603; chr19:55115863-55115887; chr19:55115906-55115930; orchr19:55116006-55116030.

In some embodiments, an engineered cell is provided, comprising a genetic modification in the AAVS1 gene, wherein the modification comprises at least 5 contiguous nucleotides within the genomic coordinates chr19:55115151-55116209. In some embodiments, the engineered cell comprises a genetic modification in the AAVS1 gene, wherein the modification comprises at least 6, 7, 8, 9, or 10 contiguous nucleotides within the genomic coordinates.

In some embodiments, an engineered cell is provided, comprising a genetic modification in the AAVS1 gene, wherein the modification comprises at least 5 contiguous nucleotides within the genomic coordinates chosen from: chr19:55115218-55115242; chr19:55115477-55115501; chr19:55115504-55115528; chr19:55115513-55115537; chr19:55115514-55115538; chr19:55115517-55115541; chr19:55115518-55115542; chr19:55115549-55115573; chr19:55115574-55115598; chr19:55115606-55115630; chr19:55115933-55115957; chr19:55116026-55116050; chr19:55116045-55116069; chr19:55116084-55116108; chr19:55115276-55115300; chr19:55115509-55115533; chr19:55115579-55115603; chr19:55115863-55115887; chr19:55115906-55115930; or chr19:55116006-55116030. In some embodiments, the engineered cell comprises a genetic modification in the AAVS1 gene, wherein the modification comprises at least 6, 7, 8, 9, or 10 contiguous nucleotides within the genomic coordinates.

In some embodiments, an engineered cell is provided, comprising a genetic modification in the AAVS1 gene, wherein the modification comprises at least one C to T substitution or at least one A to G substitution within the genomic coordinates chr19:55115151-55116209.

In some embodiments, an engineered cell is provided, comprising a genetic modification in the AAVS1 gene, wherein the modification comprises at least one C to T substitution or at least one A to G substitution within the genomic coordinates chosen from: chr19:55115218-55115242; chr19:55115477-55115501; chr19:55115504-55115528; chr19:55115513-55115537; chr19:55115514-55115538; chr19:55115517-55115541; chr19:55115518-55115542; chr19:55115549-55115573; chr19:55115574-55115598; chr19:55115606-55115630; chr19:55115933-55115957; chr19:55116026-55116050; chr19:55116045-55116069; chr19:55116084-55116108; chr19:55115276-55115300; chr19:55115509-55115533; chr19:55115579-55115603; chr19:55115863-55115887; chr19:55115906-55115930; or chr19:55116006-55116030.

In some embodiments, an engineered cell is provided that comprises a genetic modification in the AAVS1 gene, wherein the genetic modification comprises an indel, a C to T substitution, or an A to G substitution within the genomic coordinates chosen from: chr19:55115218-55115242; chr19:55115477-55115501; chr19:55115504-55115528; chr19:55115513-55115537; chr19:55115514-55115538; chr19:55115517-55115541; chr19:55115518-55115542; chr19:55115549-55115573; chr19:55115574-55115598; chr19:55115606-55115630; chr19:55115933-55115957; chr19:55116026-55116050; chr19:55116045-55116069; chr19:55116084-55116108; chr19:55115276-55115300; chr19:55115509-55115533; chr19:55115579-55115603; chr19:55115863-55115887; chr19:55115906-55115930; or chr19:55116006-55116030. In some embodiments, the genetic modification comprises at least 5 contiguous nucleotides within the genomic coordinates. In some embodiments, the genetic modification comprises at least 6, 7, 8, 9, or 10 contiguous nucleotides within the genomic coordinates. In some embodiments, the genetic modification comprises at least one C to T substitution or at least one A to G substitution within the genomic coordinates.

In some embodiments, an engineered cell is provided that has a genetic modification in AAVS1 induced by a genomic editing system that binds to an AAVS1 genomic target sequence comprising at least 10 contiguous nucleotides within the genomic coordinates chosen from: chr19:55115218-55115242; chr19:55115477-55115501; chr19:55115504-55115528; chr19:55115513-55115537; chr19:55115514-55115538; chr19:55115517-55115541; chr19:55115518-55115542; chr19:55115549-55115573; chr19:55115574-55115598; chr19:55115606-55115630; chr19:55115933-55115957; chr19:55116026-55116050; chr19:55116045-55116069; chr19:55116084-55116108; chr19:55115276-55115300; chr19:55115509-55115533; chr19:55115579-55115603; chr19:55115863-55115887; chr19:55115906-55115930; or chr19:55116006-55116030. In some embodiments, an engineered cell is provided that has a genetic modification in AAVS1 induced by a genomic editing system that binds to an AAVS1 genomic target sequence comprising at least 5 contiguous nucleotides within the genomic coordinates. In some embodiments, the AAVS1 genomic target sequence comprises at least 15 contiguous nucleotides within the genomic coordinates. In some embodiments, the genomic editing system comprises an RNA-guided DNA-binding agent. In some embodiments, the RNA-guided DNA-binding agent comprises a Cas9 protein, such as an N. meningitidis Cas9.

In some embodiments, an engineered cell is provided that has a genetic modification in AAVS1 induced by a genomic editing system that binds to an AAVS1 genomic target sequence comprising at least 5 contiguous nucleotides within the genomic coordinates chosen from: chr19:55115218-55115242; chr19:55115477-55115501; chr19:55115504-55115528; chr19:55115513-55115537; chr19:55115514-55115538; chr19:55115517-55115541; chr19:55115518-55115542; chr19:55115549-55115573; chr19:55115574-55115598; chr19:55115606-55115630; chr19:55115933-55115957; chr19:55116026-55116050; chr19:55116045-55116069; chr19:55116084-55116108; chr19:55115276-55115300; chr19:55115509-55115533; chr19:55115579-55115603; chr19:55115863-55115887; chr19:55115906-55115930; or chr19:55116006-55116030. In some embodiments, the AAVS1 genomic target sequence comprises at least 10 contiguous nucleotides within the genomic coordinates. In some embodiments, the AAVS1 genomic target sequence comprises at least 15 contiguous nucleotides within the genomic coordinates. In some embodiments, the genomic editing system comprises an RNA-guided DNA-binding agent. In some embodiments, the RNA-guided DNA-binding agent comprises a Cas9 protein, such as an N. Meningitidis.

In some embodiments, for each given range of genomic coordinates, a range may encompass +/−10 nucleotides on either end of the specified coordinates. For each given range of genomic coordinates, the range may encompass +/−5 nucleotides on either end of the range. For example, if chr16:10923222-10923242 is given, in some embodiments the genomic target sequence or genetic modification may fall within chr16:10923212-10923252.

In some embodiments, a given range of genomic coordinates may comprise a target sequence on both strands of the DNA (i.e., the plus (+) strand and the minus (−) strand).

Genetic modifications in the HLA-A, TRAC, TRBC, CIITA, and AAVS1 genes are described further herein.

In some embodiments, a genetic modification in the HLA-A, TRAC, TRBC, CIITA, or AAVS1 locus comprises any one or more of an insertion, deletion, substitution, or deamination of at least one nucleotide in a target sequence. In some embodiments, the engineered cell which has reduced or eliminated surface expression of HLA-A relative to an unmodified cell is provided, comprising a genetic modification in the HLA-A gene. In some embodiments, the engineered cell which has reduced or eliminated surface expression of TRAC relative to an unmodified cell is provided, comprising a genetic modification in the TRAC gene. In some embodiments, the engineered cell which has reduced or eliminated surface expression of TRBC1 relative to an unmodified cell is provided, comprising a genetic modification in the TRBC1 gene. In some embodiments, the engineered cell which has reduced or eliminated surface expression of TRBC2 relative to an unmodified cell is provided, comprising a genetic modification in the TRBC2 gene. In some embodiments, the engineered cell which has reduced or eliminated surface expression of MHC class II relative to an unmodified cell is provided, comprising a genetic modification in the CIITA gene.

In some embodiments, the engineered cell which has reduced or eliminated surface expression of HLA-A, TRAC, TRBC, or MHC class II relative to an unmodified cell is provided, comprising a genetic modification in the HLA-A, TRAC, TRBC, or CIITA gene, wherein the cell further has reduced or eliminated expression of an endogenous TCR protein relative to an unmodified cell. In some embodiments, the engineered cell which has reduced or eliminated surface expression of HLA-A, TRAC, TRBC, or MHC class II relative to an unmodified cell is provided, comprising a genetic modification in the HLA-A, TRAC, TRBC, or CIITA gene, wherein the cell further comprises an exogenous nucleic acid, and further has reduced or eliminated expression of an endogenous TCR protein relative to an unmodified cell. In some embodiments, the engineered cell which has reduced or eliminated surface expression of HLA-A, TRAC, TRBC, or MHC class II relative to an unmodified cell is provided, comprising a genetic modification in the HLA-A, TRAC, TRBC, or CIITA gene, wherein the cell further has reduced or eliminated surface expression of MHC class I, and wherein the cell further has reduced or eliminated expression of an endogenous TCR protein relative to an unmodified cell.

In some embodiments, the engineered cell which has reduced or eliminated surface expression of HLA-A, TRAC, TRBC, or MHC class II relative to an unmodified cell is provided, comprising a genetic modification in the HLA-A, TRAC, TRBC, or CIITA gene, wherein the cell further comprises an exogenous nucleic acid, and wherein the cell further has reduced or eliminated surface expression of MHC class I, and wherein the cell further has reduced or eliminated expression of an endogenous TCR protein relative to an unmodified cell. In some embodiments, the engineered cell has reduced or eliminated expression of a TRAC protein relative to an unmodified cell. In some embodiments, the engineered cell has reduced or eliminated expression of a TRBC protein relative to an unmodified cell.

In some embodiments, the engineered cell which has reduced or eliminated surface expression of MHC class II relative to an unmodified cell is provided, comprising a genetic modification in the CIITA gene, wherein the modification comprises at least one nucleotide of an exon within the genomic coordinates chr16:10902662-chr16:10923285, and wherein the cell further comprises an exogenous nucleic acid, and wherein the cell further has reduced or eliminated surface expression of HLA-A, and wherein the cell further has reduced or eliminated expression of an endogenous TCR protein relative to an unmodified cell. In some embodiments, the engineered cell has reduced or eliminated expression of a TRAC protein relative to an unmodified cell. In some embodiments, the engineered cell has reduced or eliminated expression of a TRBC protein relative to an unmodified cell. In some embodiments, the engineered cell comprises a genetic modification in the HLA-A gene. In some embodiments, the engineered cell comprises a genetic modification that reduces expression of HLA-A protein on the surface of the engineered cell.

The engineered cell may be any of the exemplary cell types disclosed herein. In some embodiments, the engineered cell is an immune cell. In some embodiments, the engineered cell is a hematopoetic stem cell (HSC). In some embodiments, the engineered cell is an induced pluripotent stem cell (iPSC). In some embodiments, the engineered cell is a monocyte, macrophage, mast cell, dendritic cell, or granulocyte. In some embodiments, the engineered cell is monocyte. In some embodiments, the engineered cell is a macrophage. In some embodiments, the engineered cell is a mast cell. In some embodiments, the engineered cell is a dendritic cell.

In some embodiments, the engineered cell is a granulocyte. In some embodiments, the engineered cell is a lymphocyte. In some embodiments, the engineered cell is a T cell. In some embodiments, the engineered cell is a CD4+ T cell. In some embodiments, the engineered cell is a CD8+ T cell. In some embodiments, the engineered cell is a memory T cell. In some embodiments, the engineered cell is a B cell. In some embodiments, the engineered cell is a plasma B cell. In some embodiments, the engineered cell is a memory B cell.

In some embodiments, the disclosure provides a pharmaceutical composition comprising any one of the engineered cells disclosed herein. In some embodiments, the pharmaceutical composition comprises a population of any one of the engineered cells disclosed herein. In some embodiments, the population of cells is at least 65%, at least 70%, at least 75%, at least 80%, at least 85%, at least 90%, at least 95%, at least 96%, at least 97%, at least 98%, at least 99%, or at least 99.5% negative for the surface antigen (e.g., HLA-A, MHC Class II (HLA-DP, DQ, DR), or endogenous TCR) as measured by flow cytometry. In some embodiments, the population of engineered cells that is at least 65% negative as measured by flow cytometry. In some embodiments, the population of engineered cells that is at least 70% negative as measured by flow cytometry. In some embodiments, the population of engineered cells is at least 80% negative as measured by flow cytometry. In some embodiments, the population of engineered cells is at least 90% negative as measured by flow cytometry. In some embodiments, the population of engineered cells is at least 91% negative as measured by flow cytometry. In some embodiments, the population of engineered cells is at least 92% negative as measured by flow cytometry. In some embodiments, the population of engineered cells is at least 93% negative as measured by flow cytometry. In some embodiments, the population of engineered cells is at least 94% negative as measured by flow cytometry. In some embodiments, the population of engineered cells is at least 95% endogenous TCR protein negative as measured by flow cytometry. In some embodiments, the population of engineered cells is at least 97% endogenous TCR protein negative as measured by flow cytometry. In some embodiments, the population of engineered cells is at least 98% endogenous TCR protein negative as measured by flow cytometry. In some embodiments, the population of engineered cells is at least 99% endogenous TCR protein negative as measured by flow cytometry.

In some embodiments, methods are provided for administering the engineered cells or pharmaceutical compositions disclosed herein to a subject in need thereof. In some embodiments, methods are provided for administering the engineered cells or pharmaceutical compositions disclosed herein to a subject as an ACT therapy. In some embodiments, methods are provided for administering the engineered cells or pharmaceutical compositions disclosed herein to a subject as a treatment for cancer. In some embodiments, methods are provided for administering the engineered cells or pharmaceutical compositions disclosed herein to a subject as a treatment for an autoimmune disease. In some embodiments, methods are provided for administering the engineered cells or pharmaceutical compositions disclosed herein to a subject as a treatment for an infectious disease.

### B. Methods and Compositions for Reducing or Eliminating Surface Expression of HLA-A, TRAC, TRBC, and MHC Class II

The present disclosure provides methods and compositions for reducing or eliminating surface expression of HLA-A, TRAC, TRBC, or MHC class II protein on a cell relative to an unmodified cell by genetically modifying the HLA-A, TRAC, TRBC, or CIITA gene. The resultant genetically modified cell may also be referred to herein as an engineered cell. In some embodiments, an already-genetically modified (or engineered) cell may be the starting cell for further genetic modification using the methods or compositions provided herein. In some embodiments, the cell is an allogeneic cell. In some embodiments, a cell with reduced HLA-A, TRAC, TRBC, or MHC class II expression is useful for adoptive cell transfer therapies. In some embodiments, editing of the HLA-A, TRAC, TRBC, or CIITA gene is combined with additional genetic modifications to yield a cell that is desirable for allogeneic transplant purposes.

In some embodiments, the methods comprise reducing or eliminating surface expression of HLA-A protein on the surface of a cell comprising contacting a cell with a composition comprising an HLA-A guide RNA comprising a guide sequence that targets an HLA-A genomic target sequence comprising at least 10 contiguous nucleotides within the genomic coordinates chr6:29942540-29945459. In some embodiments, the methods further comprise contacting the cell with an RNA-guided DNA binding agent or a nucleic acid encoding an RNA-guided DNA binding agent. In some embodiments, the RNA-guided DNA binding agent is Cas9. In some embodiments, the RNA-guided DNA binding agent is N. meningitidis Cas9. In some embodiments, the HLA-A guide RNA is a N. meningitidis Cas9 guide RNA. In some embodiments, the RNA-guided DNA binding agent comprises a deaminase domain. In some embodiments the RNA-guided DNA binding agent comprises an APOBEC3A deaminase (A3A) and an RNA-guided nickase. In some embodiments, the expression of HLA-A protein on the surface of the cell (i.e., engineered cell) is thereby reduced. In some embodiments, the HLA-A guide RNA comprises a guide sequence selected from SEQ ID NO: 2-80. In some embodiments, the methods comprise making an engineered cell, which has reduced or eliminated surface expression of HLA-A protein relative to an unmodified cell, comprising contact the cell with a composition comprising an HLA-A guide RNA comprising a guide sequence that targets a genomic target comprising at least 10 contiguous nucleotides within the genomic coordinates chr6:29942540-29945459. In some embodiments, the methods further comprise contacting the cell with an RNA-guided DNA binding agent or a nucleic acid encoding an RNA-guided DNA binding agent. In some embodiments, the RNA-guided DNA binding agent is Cas9. In some embodiments, the RNA-guided DNA binding agent is N. meningitidis Cas9. In some embodiments, the HLA-A guide RNA is a N. meningitidis Cas9 guide RNA. In some embodiments, the RNA-guided DNA binding agent comprises a deaminase region. In some embodiments the RNA-guided DNA binding agent comprises an APOBEC3A deaminase (A3A) and an RNA-guided nickase. In some embodiments, the expression of HLA-A protein on the surface of the cell (i.e., engineered cell) is thereby reduced. In some embodiments, the HLA-A guide RNA comprises a guide sequence selected from SEQ ID NO: 2-80.

In some embodiments, the methods comprise genetically modifying a cell to reduce or eliminate the surface expression of HLA-A protein comprising contacting the cell with a composition comprising an HLA-A guide RNA comprising a guide sequence that targets a genomic target comprising at least 10 contiguous nucleotides within the genomic coordinates chr6:29942540-29945459. In some embodiments, the methods further comprise contacting the cell with an RNA-guided DNA binding agent or a nucleic acid encoding an RNA-guided DNA binding agent. In some embodiments, the RNA-guided DNA binding agent is Cas9. In some embodiments, the RNA-guided DNA binding agent is N. meningitidis Cas9. In some embodiments, the HLA-A guide RNA is a N. meningitidis Cas9 guide RNA. In some embodiments, the RNA-guided DNA binding agent comprises a deaminase region. In some embodiments the RNA-guided DNA binding agent comprises an APOBEC3A deaminase (A3A) and an RNA-guided nickase. In some embodiments, the expression of HLA-A protein on the surface of the cell (i.e., engineered cell) is thereby reduced. In some embodiments, the HLA-A guide RNA comprises a guide sequence selected from SEQ ID NO: 2-80.

In some embodiments, the methods comprise genetically modifying HLA-A comprising contacting a cell with a composition comprising an HLA-A guide RNA comprising a guide sequence that targets a genomic target comprising at least 10 contiguous nucleotides within the genomic coordinates chr6:29942540-29945459. In some embodiments, the methods further comprise contacting the cell with an RNA-guided DNA binding agent or a nucleic acid encoding an RNA-guided DNA binding agent. In some embodiments, the RNA-guided DNA binding agent is Cas9. In some embodiments, the RNA-guided DNA binding agent is N. meningitidis Cas9. In some embodiments, the HLA-A guide RNA is a N. meningitidis Cas9 guide RNA. In some embodiments, the RNA-guided DNA binding agent comprises a deaminase region. In some embodiments the RNA-guided DNA binding agent comprises an APOBEC3A deaminase (A3A) and an RNA-guided nickase. In some embodiments, the expression of HLA-A protein on the surface of the cell (i.e., engineered cell) is thereby reduced. In some embodiments, the HLA-A guide RNA comprises a guide sequence selected from SEQ ID NO: 2-80.

In some embodiments, the methods comprise inducing a DSB or a single stranded break (SSB) in HLA-A comprising contacting a cell with a composition comprising an HLA-A guide RNA comprising a guide sequence targets a genomic target comprising at least 10 contiguous nucleotides within the genomic coordinates chr6:29942540-29945459. In some embodiments, the methods further comprise contacting the cell with an RNA-guided DNA binding agent or a nucleic acid encoding an RNA-guided DNA binding agent. In some embodiments, the RNA-guided DNA binding agent is Cas9. In some embodiments, the RNA-guided DNA binding agent is S. pyogenes Cas9. In some embodiments, the HLA-A guide RNA is a N. meningitidis Cas9 guide RNA. In some embodiments, the RNA-guided DNA binding agent comprises a deaminase region. In some embodiments the RNA-guided DNA binding agent comprises an APOBEC3A deaminase (A3A) and an RNA-guided nickase. In some embodiments, the expression of HLA-A protein on the surface of the cell (i.e., engineered cell) is thereby reduced. In some embodiments, the HLA-A guide RNA comprises a guide sequence selected from SEQ ID NO: 2-80.

In some embodiments, the methods comprise reducing expression of the HLA-A protein in a cell comprising delivering a composition to a cell comprising contacting a cell with a composition comprising an HLA-A guide RNA comprising a guide sequence targets a genomic target comprising at least 10 contiguous nucleotides within the genomic coordinates chr6:29942540-29945459. In some embodiments, the methods further comprise contacting the cell with an RNA-guided DNA binding agent or a nucleic acid encoding an RNA-guided DNA binding agent. In some embodiments, the RNA-guided DNA binding agent is Cas9. In some embodiments, the RNA-guided DNA binding agent is N. meningitidis Cas9. In some embodiments, the HLA-A guide RNA is a N. meningitidis Cas9 guide RNA. In some embodiments, the RNA-guided DNA binding agent comprises a deaminase region. In some embodiments the RNA-guided DNA binding agent comprises an APOBEC3A deaminase (A3A) and an RNA-guided nickase. In some embodiments, the expression of HLA-A protein on the surface of the cell (i.e., engineered cell) is thereby reduced. In some embodiments, the HLA-A guide RNA comprises a guide sequence selected from SEQ ID NO: 2-80.

In some embodiments, the methods of reducing expression of an HLA-A protein on the surface of a cell comprise contacting a cell with any one or more of the HLA-A guide RNAs disclosed herein. In some embodiments, the HLA-A guide RNA comprises a guide sequence selected from SEQ ID NO: 2-80.

In some embodiments, compositions are provided comprising an HLA-A guide RNA comprising a guide sequence that targets a genomic target comprising at least 10 contiguous nucleotides within the genomic coordinates chr6:29942540-29945459. In some embodiments, the composition further comprises an RNA-guided DNA binding agent or a nucleic acid encoding an RNA-guided DNA binding agent. In some embodiments, the composition comprises an RNA-guided DNA binding agent that is Cas9. In some embodiments, the RNA-guided DNA binding agent is N. meningitidis Cas9. In some embodiments, the HLA-A guide RNA is a N. meningitidis Cas9 guide RNA. In some embodiments, the RNA-guided DNA binding agent comprises a deaminase region. In some embodiments the RNA-guided DNA binding agent comprises an APOBEC3A deaminase (A3A) and an RNA-guided nickase. In some embodiments, the HLA-A guide RNA comprises a guide sequence selected from SEQ ID NO: 2-80.

In some embodiments, a composition is provided, the composition comprising: a) an HLA-A guide RNA (gRNA) comprising i) a guide sequence selected from SEQ ID NOs: 2-80; or ii) at least 19, 20, 21, 22, 23, or 24 contiguous nucleotides of a sequence selected from SEQ ID NOs: 2-80; or iii) a guide sequence at least 95%, 90%, or 85% identical to a sequence selected from SEQ ID NOs: 2-80; or iv) a sequence that comprises 10 contiguous nucleotides±10 nucleotides of a genomic coordinate listed in Table 1; or v) at least 19, 20, 21, 22, 23, or 24 contiguous nucleotides of a sequence from (iv); or vi) a guide sequence that is at least 95%, 90%, or 85% identical to a sequence selected from (iv). In some embodiments, the HLA-A guide RNA (gRNA) is a single guide RNA.

In some embodiments, a method of making an engineered cell, which has reduced or eliminated surface expression of HLA-A protein relative to an unmodified cell, is provided, the method comprising contacting a cell with a composition of any of the embodiments provided herein. In some embodiments, the composition comprises an HLA-A guide RNA, comprising a guide sequence of any one of: SEQ ID NOs: 1, 13, 55, 61, 66, 70, and 71. In some embodiments, the composition comprises an HLA-A guide RNA, comprising a guide sequence of any one of: SEQ ID NOs: 13, 55, 61, 66, 70, and 71. In some embodiments, the composition comprises an HLA-A guide RNA, comprising a guide sequence of any one of: SEQ ID NOs: 13, 17, 55, 61, 66, and 70.

In some embodiments, a method of reducing surface expression of HLA-A protein in an engineered cell relative to an unmodified cell, is provided, the method comprising contacting a cell with a composition of any of embodiments provided herein.

In some embodiments, the composition further comprises a uracil glycosylase inhibitor (UGI). In some embodiments, the composition comprises an RNA-guided DNA binding agent that the RNA-guided DNA binding agent generates a cytosine (C) to thymine (T) conversion with the HLA-A genomic target sequence. In some embodiments, the composition comprises an RNA-guided DNA binding agent that generates a adenosine (A) to guanine (G) conversion with the HLA-A genomic target sequence.

In some embodiments, an engineered cell produced by the methods described herein is provided. In some embodiments, the engineered cell produced by the methods and compositions described herein is an allogeneic cell. In some embodiments, the methods produce a composition comprising an engineered cell having reduced HLA-A expression. In some embodiments, the methods produce a composition comprising an engineered cell having reduced HLA-A protein expression. In some embodiments, the methods produce a composition comprising an engineered cell having reduced HLA-A levels in the cell nucleus. In some embodiments, the methods produce a composition comprising an engineered cell that expresses a truncated form of the HLA-A protein. In some embodiments, the methods produce a composition comprising an engineered cell that produces no detectable HLA-A protein. In some embodiments, the engineered cell has reduced HLA-A expression, reduced HLA-A protein, or reduced HLA-A levels in the cell nucleus as compared to an unmodified cell. In some embodiments, the engineered cell produced by the methods disclosed herein elicits a reduced response from CD4+ T cells as compared to an unmodified cell as measured in an in vitro cell culture assay containing CD4+ T cells.

In some embodiments, an engineered cell produced by the methods or compositions disclosed herein is provided wherein the cell has reduced or eliminated surface expression of HLA-A protein and wherein the cell comprises a genetic modification comprising at least 5 contiguous nucleotides within the genomic coordinates chr6:29942540-29945459. In some embodiments, an engineered cell produced by the methods or compositions disclosed herein is provided wherein the cell has reduced or eliminated surface expression of HLA-A protein and wherein the cell comprises a genetic modification comprising at least 10 contiguous nucleotides within the genomic coordinates chr6:29942540-29945459. In some embodiments, an engineered cell produced by the methods or compositions disclosed herein is provided wherein the cell has reduced or eliminated surface expression of HLA-A protein and wherein the cell comprises a genetic modification comprising at least one C to T substitution or at least one A to G substitution within the genomic coordinates chr6:29942540-29945459.

In some embodiments, the methods comprise reducing or eliminating surface expression of TRAC protein on the surface of a cell comprising contacting a cell with a composition comprising a TRAC guide RNA comprising a guide sequence that targets a genomic target comprising at least 10 contiguous nucleotides within the genomic coordinates chr14:22547505-22551621 or chr14:22547462-22551621. In some embodiments, the methods further comprise contacting the cell with an RNA-guided DNA binding agent or a nucleic acid encoding an RNA-guided DNA binding agent. In some embodiments, the RNA-guided DNA binding agent is Cas9. In some embodiments, the RNA-guided DNA binding agent is N. meningitidis Cas9. In some embodiments, the TRAC guide RNA is a N. meningitidis Cas9 guide RNA. In some embodiments, the RNA-guided DNA binding agent comprises a deaminase domain. In some embodiments the RNA-guided DNA binding agent comprises an APOBEC3A deaminase (A3A) and an RNA-guided nickase. In some embodiments, the expression of TRAC protein on the surface of the cell (i.e., engineered cell) is thereby reduced. In some embodiments, the TRAC guide RNA comprises a guide sequence selected from SEQ ID NO: 101-120.

In some embodiments, the methods comprise making an engineered cell, which has reduced or eliminated surface expression of TRAC protein relative to an unmodified cell, comprising contact the cell with a composition comprising a TRAC guide RNA comprising a guide sequence that targets a genomic target comprising at least 10 contiguous nucleotides within the genomic coordinates chr14:22547505-22551621 or chr14:22547462-22551621. In some embodiments, the methods further comprise contacting the cell with an RNA-guided DNA binding agent or a nucleic acid encoding an RNA-guided DNA binding agent. In some embodiments, the RNA-guided DNA binding agent is Cas9. In some embodiments, the RNA-guided DNA binding agent is N. meningitidis Cas9. In some embodiments, the TRAC guide RNA is a N. meningitidis Cas9 guide RNA. In some embodiments, the RNA-guided DNA binding agent comprises a deaminase region. In some embodiments the RNA-guided DNA binding agent comprises an APOBEC3A deaminase (A3A) and an RNA-guided nickase. In some embodiments, the expression of TRAC protein on the surface of the cell (i.e., engineered cell) is thereby reduced. In some embodiments, the TRAC guide RNA comprises a guide sequence selected from SEQ ID NO: 101-120.

In some embodiments, the methods comprise genetically modifying a cell to reduce or eliminate the surface expression of TRAC protein comprising contacting the cell with a composition comprising a TRAC guide RNA comprising a guide sequence that targets a genomic target comprising at least 10 contiguous nucleotides within the genomic coordinates chr14:22547505-22551621 or chr14:22547462-22551621. In some embodiments, the methods further comprise contacting the cell with an RNA-guided DNA binding agent or a nucleic acid encoding an RNA-guided DNA binding agent. In some embodiments, the RNA-guided DNA binding agent is Cas9. In some embodiments, the RNA-guided DNA binding agent is N. meningitidis Cas9. In some embodiments, the TRAC guide RNA is a N. meningitidis Cas9 guide RNA. In some embodiments, the RNA-guided DNA binding agent comprises a deaminase region. In some embodiments the RNA-guided DNA binding agent comprises an APOBEC3A deaminase (A3A) and an RNA-guided nickase. In some embodiments, the expression of TRAC protein on the surface of the cell (i.e., engineered cell) is thereby reduced. In some embodiments, the TRAC guide RNA comprises a guide sequence selected from SEQ ID NO: 101-120.

In some embodiments, the methods comprise genetically modifying TRAC comprising contacting a cell with a composition comprising a TRAC guide RNA comprising a guide sequence that targets a genomic target comprising at least 10 contiguous nucleotides within the genomic coordinates chr14:22547505-22551621 or chr14:22547462-22551621. In some embodiments, the methods further comprise contacting the cell with an RNA-guided DNA binding agent or a nucleic acid encoding an RNA-guided DNA binding agent. In some embodiments, the RNA-guided DNA binding agent is Cas9. In some embodiments, the RNA-guided DNA binding agent is N. meningitidis Cas9. In some embodiments, the TRAC guide RNA is a N. meningitidis Cas9 guide RNA. In some embodiments, the RNA-guided DNA binding agent comprises a deaminase region. In some embodiments the RNA-guided DNA binding agent comprises an APOBEC3A deaminase (A3A) and an RNA-guided nickase. In some embodiments, the expression of TRAC protein on the surface of the cell (i.e., engineered cell) is thereby reduced. In some embodiments, the TRAC guide RNA comprises a guide sequence selected from SEQ ID NO: 101-120.

In some embodiments, the methods comprise inducing a DSB or a single stranded break (SSB) in TRAC comprising contacting a cell with a composition comprising a TRAC guide RNA comprising a guide sequence targets a genomic target comprising at least 10 contiguous nucleotides within the genomic coordinates chr14:22547505-22551621 or chr14:22547462-22551621. In some embodiments, the methods further comprise contacting the cell with an RNA-guided DNA binding agent or a nucleic acid encoding an RNA-guided DNA binding agent. In some embodiments, the RNA-guided DNA binding agent is Cas9. In some embodiments, the RNA-guided DNA binding agent is S. pyogenes Cas9. In some embodiments, the TRAC guide RNA is a N. meningitidis Cas9 guide RNA. In some embodiments, the RNA-guided DNA binding agent comprises a deaminase region. In some embodiments the RNA-guided DNA binding agent comprises an APOBEC3A deaminase (A3A) and an RNA-guided nickase. In some embodiments, the expression of TRAC protein on the surface of the cell (i.e., engineered cell) is thereby reduced. In some embodiments, the TRAC guide RNA comprises a guide sequence selected from SEQ ID NO: 101-120.

In some embodiments, the methods comprise reducing expression of the TRAC protein in a cell comprising delivering a composition to a cell comprising contacting a cell with a composition comprising a TRAC guide RNA comprising a guide sequence targets a genomic target comprising at least 10 contiguous nucleotides within the genomic coordinates chr14:22547505-22551621 or chr14:22547462-22551621. In some embodiments, the methods further comprise contacting the cell with an RNA-guided DNA binding agent or a nucleic acid encoding an RNA-guided DNA binding agent. In some embodiments, the RNA-guided DNA binding agent is Cas9. In some embodiments, the RNA-guided DNA binding agent is N. meningitidis Cas9. In some embodiments, the TRAC guide RNA is a N. meningitidis Cas9 guide RNA. In some embodiments, the RNA-guided DNA binding agent comprises a deaminase region. In some embodiments the RNA-guided DNA binding agent comprises an APOBEC3A deaminase (A3A) and an RNA-guided nickase. In some embodiments, the expression of TRAC protein on the surface of the cell (i.e., engineered cell) is thereby reduced. In some embodiments, the TRAC guide RNA comprises a guide sequence selected from SEQ ID NO: 101-120.

In some embodiments, the methods of reducing expression of a TRAC protein on the surface of a cell comprise contacting a cell with any one or more of the TRAC guide RNAs disclosed herein. In some embodiments, the TRAC guide RNA comprises a guide sequence selected from SEQ ID NO: 101-120.

In some embodiments, compositions are provided comprising a TRAC guide RNA comprising a guide sequence that targets a genomic target comprising at least 10 contiguous nucleotides within the genomic coordinates chr14:22547505-22551621 or chr14:22547462-22551621. In some embodiments, the composition further comprises an RNA-guided DNA binding agent or a nucleic acid encoding an RNA-guided DNA binding agent. In some embodiments, the composition comprises an RNA-guided DNA binding agent that is Cas9. In some embodiments, the RNA-guided DNA binding agent is N. meningitidis Cas9. In some embodiments, the TRAC guide RNA is a N. meningitidis Cas9 guide RNA. In some embodiments, the RNA-guided DNA binding agent comprises a deaminase region. In some embodiments the RNA-guided DNA binding agent comprises an APOBEC3A deaminase (A3A) and an RNA-guided nickase. In some embodiments, the TRAC guide RNA comprises a guide sequence selected from SEQ ID NO: 101-120.

In some embodiments, a composition is provided, the composition comprising: a) a TRAC guide RNA (gRNA) comprising i) a guide sequence selected from SEQ ID NOs: 101-120; or ii) at least 19, 20, 21, 22, 23, or 24 contiguous nucleotides of a sequence selected from SEQ ID NOs: 101-120; or iii) a guide sequence at least 95%, 90%, or 85% identical to a sequence selected from SEQ ID NOs: 101-120; or iv) a sequence that comprises 10 contiguous nucleotides±10 nucleotides of a genomic coordinate listed in Table 2; or v) at least 19, 20, 21, 22, 23, or 24 contiguous nucleotides of a sequence from (iv); or vi) a guide sequence that is at least 95%, 90%, or 85% identical to a sequence selected from (v). In some embodiments, the TRAC guide RNA (gRNA) is a single-guide RNA (sgRNA).

In some embodiments, a method of making an engineered cell, which has reduced or eliminated surface expression of TRAC protein relative to an unmodified cell, is provided, the method comprising contacting a cell with a composition of any of the embodiments provided herein. In some embodiments, the composition comprises a TRAC guide RNA, comprising a guide sequence of any one of: SEQ ID NO: 101, 102, 103, 105, 107, 109, 111, and 115. In some embodiments, the composition comprises a TRAC guide RNA comprising a guide sequence of any one of: SEQ ID NO: 101, 102, 103, 107, and 111.

In some embodiments, a method of reducing surface expression of TRAC protein in an engineered cell relative to an unmodified cell, is provided, the method comprising contacting a cell with a composition of any of embodiments provided herein.
embodiments, the composition further comprises a uracil glycosylase inhibitor (UGI). In some embodiments, the composition comprises an RNA-guided DNA binding agent that the RNA-guided DNA binding agent generates a cytosine (C) to thymine (T) conversion with the TRAC genomic target sequence. In some embodiments, the composition comprises an RNA-guided DNA binding agent that generates a adenosine (A) to guanine (G) conversion with the TRAC genomic target sequence.

In some embodiments, an engineered cell produced by the methods described herein is provided. In some embodiments, the engineered cell produced by the methods and compositions described herein is an allogeneic cell. In some embodiments, the methods produce a composition comprising an engineered cell having reduced TRAC expression. In some embodiments, the methods produce a composition comprising an engineered cell having reduced TRAC protein expression. In some embodiments, the methods produce a composition comprising an engineered cell having reduced TRAC levels in the cell nucleus. In some embodiments, the methods produce a composition comprising an engineered cell that expresses a truncated form of the TRAC protein. In some embodiments, the methods produce a composition comprising an engineered cell that produces no detectable TRAC protein. In some embodiments, the engineered cell has reduced TRAC expression, reduced TRAC protein, or reduced TRAC levels in the cell nucleus as compared to an unmodified cell. In some embodiments, the engineered cell produced by the methods disclosed herein elicits a reduced response from CD4+ T cells as compared to an unmodified cell as measured in an in vitro cell culture assay containing CD4+ T cells.

In some embodiments, an engineered cell produced by the methods or compositions disclosed herein is provided wherein the cell has reduced or eliminated surface expression of TRAC protein and wherein the cell comprises a genetic modification comprising at least 5 contiguous nucleotides within the genomic coordinates chr14:22547505-22551621 or chr14:22547462-22551621. In some embodiments, an engineered cell produced by the methods or compositions disclosed herein is provided wherein the cell has reduced or eliminated surface expression of TRAC protein and wherein the cell comprises a genetic modification comprising at least 10 contiguous nucleotides within the genomic coordinates chr14:22547505-22551621 or chr14:22547462-22551621. In some embodiments, an engineered cell produced by the methods or compositions disclosed herein is provided wherein the cell has reduced or eliminated surface expression of TRAC protein and wherein the cell comprises a genetic modification comprising at least one C to T substitution or at least one A to G substitution within the genomic coordinates chr14:22547505-22551621 or chr14:22547462-22551621.

In some embodiments, the methods comprise reducing or eliminating surface expression of TRBC protein on the surface of a cell comprising contacting a cell with a composition comprising a TRBC guide RNA comprising a guide sequence that targets a genomic target comprising at least 10 contiguous nucleotides within the genomic coordinates chosen from chr7:142791756-142802543. In some embodiments, the methods comprise reducing or eliminating surface expression of TRBC protein on the surface of a cell comprising contacting a cell with a composition comprising a TRBC guide RNA comprising a guide sequence that targets a genomic target comprising at least 10 contiguous nucleotides within the genomic coordinates chosen from: (a) chr7:142791862-142793149; (b) chr7: 142791756-142792721; or (c) chr7:142801104-142802543. In some embodiments, the methods further comprise contacting the cell with an RNA-guided DNA binding agent or a nucleic acid encoding an RNA-guided DNA binding agent. In some embodiments, the RNA-guided DNA binding agent is Cas9. In some embodiments, the RNA-guided DNA binding agent is N. meningitidis Cas9. In some embodiments, the TRBC guide RNA is a N. meningitidis Cas9 guide RNA. In some embodiments, the RNA-guided DNA binding agent comprises a deaminase domain. In some embodiments the RNA-guided DNA binding agent comprises an APOBEC3A deaminase (A3A) and an RNA-guided nickase. In some embodiments, the expression of TRBC protein on the surface of the cell (i.e., engineered cell) is thereby reduced. In some embodiments, the TRBC guide RNA comprises a guide sequence selected from SEQ ID NO: 201-265.

In some embodiments, the methods comprise making an engineered cell, which has reduced or eliminated surface expression of TRBC protein relative to an unmodified cell, comprising contact the cell with a composition comprising a TRBC guide RNA comprising a guide sequence that targets a genomic target comprising at least 10 contiguous nucleotides within the genomic coordinates coordinates chosen from: (a) chr7:142791862-142793149; (b) chr7: 142791756-142792721; or (c) chr7:142801104-142802543. In some embodiments, the methods further comprise contacting the cell with an RNA-guided DNA binding agent or a nucleic acid encoding an RNA-guided DNA binding agent. In some embodiments, the RNA-guided DNA binding agent is Cas9. In some embodiments, the RNA-guided DNA binding agent is N. meningitidis Cas9. In some embodiments, the TRBC guide RNA is a N. meningitidis Cas9 guide RNA. In some embodiments, the RNA-guided DNA binding agent comprises a deaminase region. In some embodiments the RNA-guided DNA binding agent comprises an APOBEC3A deaminase (A3A) and an RNA-guided nickase. In some embodiments, the expression of TRBC protein on the surface of the cell (i.e., engineered cell) is thereby reduced. In some embodiments, the TRBC guide RNA comprises a guide sequence selected from SEQ ID NO: 201-265.

In some embodiments, the methods comprise genetically modifying a cell to reduce or eliminate the surface expression of TRBC protein comprising contacting the cell with a composition comprising a TRBC guide RNA comprising a guide sequence that targets a genomic target comprising at least 10 contiguous nucleotides within the genomic coordinates chosen from: (a) chr7:142791862-142793149; (b) chr7: 142791756-142792721; or (c) chr7:142801104-142802543. In some embodiments, the methods further comprise contacting the cell with an RNA-guided DNA binding agent or a nucleic acid encoding an RNA-guided DNA binding agent. In some embodiments, the RNA-guided DNA binding agent is Cas9. In some embodiments, the RNA-guided DNA binding agent is N. meningitidis Cas9. In some embodiments, the TRBC guide RNA is a N. meningitidis Cas9 guide RNA. In some embodiments, the RNA-guided DNA binding agent comprises a deaminase region. In some embodiments the RNA-guided DNA binding agent comprises an APOBEC3A deaminase (A3A) and an RNA-guided nickase. In some embodiments, the expression of TRBC protein on the surface of the cell (i.e., engineered cell) is thereby reduced. In some embodiments, the TRBC guide RNA comprises a guide sequence selected from SEQ ID NO: 201-265.

In some embodiments, the methods comprise genetically modifying TRBC comprising contacting a cell with a composition comprising a TRBC guide RNA comprising a guide sequence that targets a genomic target comprising at least 10 contiguous nucleotides within the genomic coordinates coordinates chosen from: (a) chr7:142791862-142793149; (b) chr7: 142791756-142792721; or (c) chr7:142801104-142802543. In some embodiments, the methods further comprise contacting the cell with an RNA-guided DNA binding agent or a nucleic acid encoding an RNA-guided DNA binding agent. In some embodiments, the RNA-guided DNA binding agent is Cas9. In some embodiments, the RNA-guided DNA binding agent is N. meningitidis Cas9. In some embodiments, the TRBC guide RNA is a N. meningitidis Cas9 guide RNA. In some embodiments, the RNA-guided DNA binding agent comprises a deaminase region. In some embodiments the RNA-guided DNA binding agent comprises an APOBEC3A deaminase (A3A) and an RNA-guided nickase. In some embodiments, the expression of TRBC protein on the surface of the cell (i.e., engineered cell) is thereby reduced. In some embodiments, the TRBC guide RNA comprises a guide sequence selected from SEQ ID NO: 201-265.

In some embodiments, the methods comprise inducing a DSB or a single stranded break (SSB) in TRBC comprising contacting a cell with a composition comprising a TRBC guide RNA comprising a guide sequence targets a genomic target comprising at least 10 contiguous nucleotides within the genomic coordinates coordinates chosen from: (a) chr7:142791862-142793149; (b) chr7: 142791756-142792721; or (c) chr7:142801104-142802543. In some embodiments, the methods further comprise contacting the cell with an RNA-guided DNA binding agent or a nucleic acid encoding an RNA-guided DNA binding agent. In some embodiments, the RNA-guided DNA binding agent is Cas9. In some embodiments, the RNA-guided DNA binding agent is S. pyogenes Cas9. In some embodiments, the TRBC guide RNA is a N. meningitidis Cas9 guide RNA. In some embodiments, the RNA-guided DNA binding agent comprises a deaminase region. In some embodiments the RNA-guided DNA binding agent comprises an APOBEC3A deaminase (A3A) and an RNA-guided nickase. In some embodiments, the expression of TRBC protein on the surface of the cell (i.e., engineered cell) is thereby reduced. In some embodiments, the TRBC guide RNA comprises a guide sequence selected from SEQ ID NO: 201-265.

In some embodiments, the methods comprise reducing expression of the TRBC protein in a cell comprising delivering a composition to a cell comprising contacting a cell with a composition comprising a TRBC guide RNA comprising a guide sequence targets a genomic target comprising at least 10 contiguous nucleotides within the genomic coordinates coordinates chosen from: (a) chr7:142791862-142793149; (b) chr7: 142791756-142792721; or (c) chr7:142801104-142802543. In some embodiments, the methods further comprise contacting the cell with an RNA-guided DNA binding agent or a nucleic acid encoding an RNA-guided DNA binding agent. In some embodiments, the RNA-guided DNA binding agent is Cas9. In some embodiments, the RNA-guided DNA binding agent is N. meningitidis Cas9. In some embodiments, the TRBC guide RNA is a N. meningitidis Cas9 guide RNA. In some embodiments, the RNA-guided DNA binding agent comprises a deaminase region. In some embodiments the RNA-guided DNA binding agent comprises an APOBEC3A deaminase (A3A) and an RNA-guided nickase. In some embodiments, the expression of TRBC protein on the surface of the cell (i.e., engineered cell) is thereby reduced. In some embodiments, the TRBC guide RNA comprises a guide sequence selected from SEQ ID NO: 201-265.

In some embodiments, the methods of reducing expression of an TRBC protein on the surface of a cell comprise contacting a cell with any one or more of the TRBC guide RNAs disclosed herein. In some embodiments, the TRBC guide RNA comprises a guide sequence selected from SEQ ID NO: 201-265.

In some embodiments, compositions are provided comprising a TRBC guide RNA comprising a guide sequence that targets a genomic target comprising at least 10 contiguous nucleotides within the genomic coordinates chosen from: (a) chr7:142791862-142793149; (b) chr7: 142791756-142792721; or (c) chr7:142801104-142802543. In some embodiments, the composition further comprises an RNA-guided DNA binding agent or a nucleic acid encoding an RNA-guided DNA binding agent. In some embodiments, the composition comprises an RNA-guided DNA binding agent that is Cas9. In some embodiments, the RNA-guided DNA binding agent is N. meningitidis Cas9. In some embodiments, the TRBC guide RNA is a N. meningitidis Cas9 guide RNA. In some embodiments, the RNA-guided DNA binding agent comprises a deaminase region. In some embodiments the RNA-guided DNA binding agent comprises an APOBEC3A deaminase (A3A) and an RNA-guided nickase. In some embodiments, the TRBC guide RNA comprises a guide sequence selected from SEQ ID NO: 201-265.

In some embodiments, a composition is provided, the composition comprising: a) a TRBC guide RNA (gRNA) comprising i) a guide sequence selected from SEQ ID NOs: 201-265; or ii) at least 19, 20, 21, 22, 23, or 24 contiguous nucleotides of a sequence selected from SEQ ID NOs: 201-265; or iii) a guide sequence at least 95%, 90%, or 85% identical to a sequence selected from SEQ ID NOs: 201-265; or iv) a sequence that comprises 10 contiguous nucleotides±10 nucleotides of a genomic coordinate listed in Table 3; or v) at least 19, 20, 21, 22, 23, or 24 contiguous nucleotides of a sequence from (iv); or vi) a guide sequence that is at least 95%, 90%, or 85% identical to a sequence selected from (v). In some embodiments, the TRBC guide RNA that is a single-guide RNA (sgRNA).

In some embodiments, the composition further comprises a uracil glycosylase inhibitor (UGI). In some embodiments, the composition comprises an RNA-guided DNA binding agent that the RNA-guided DNA binding agent generates a cytosine (C) to thymine (T) conversion with the TRBC genomic target sequence. In some embodiments, the composition comprises an RNA-guided DNA binding agent that generates a adenosine (A) to guanine (G) conversion with the TRBC genomic target sequence.

In some embodiments, an engineered cell produced by the methods described herein is provided. In some embodiments, the engineered cell produced by the methods and compositions described herein is an allogeneic cell. In some embodiments, the methods produce a composition comprising an engineered cell having reduced TRBC expression. In some embodiments, the methods produce a composition comprising an engineered cell having reduced TRBC protein expression. In some embodiments, the methods produce a composition comprising an engineered cell having reduced TRBC levels in the cell nucleus. In some embodiments, the methods produce a composition comprising an engineered cell that expresses a truncated form of the TRBC protein. In some embodiments, the methods produce a composition comprising an engineered cell that produces no detectable TRBC protein. In some embodiments, the engineered cell has reduced TRBC expression, reduced TRBC protein, or reduced TRBC levels in the cell nucleus as compared to an unmodified cell. In some embodiments, the engineered cell produced by the methods disclosed herein elicits a reduced response from CD4+ T cells as compared to an unmodified cell as measured in an in vitro cell culture assay containing CD4+ T cells.

In some embodiments, an engineered cell produced by the methods or compositions disclosed herein is provided wherein the cell has reduced or eliminated surface expression of TRBC protein and wherein the cell comprises a genetic modification comprising at least 5 contiguous nucleotides within the genomic coordinates chosen from: (a) chr7:142791862-142793149; (b) chr7: 142791756-142792721; or (c) chr7:142801104-142802543. In some embodiments, an engineered cell produced by the methods or compositions disclosed herein is provided wherein the cell has reduced or eliminated surface expression of TRBC protein and wherein the cell comprises a genetic modification comprising at least 10 contiguous nucleotides within the genomic coordinates chosen from: (a) chr7:142791862-142793149; (b) chr7: 142791756-142792721; or (c) chr7:142801104-142802543. In some embodiments, an engineered cell produced by the methods or compositions disclosed herein is provided wherein the cell has reduced or eliminated surface expression of TRBC protein and wherein the cell comprises a genetic modification comprising at least one C to T substitution or at least one A to G substitution within the genomic coordinates chosen from: (a) chr7:142791862-142793149; (b) chr7: 142791756-142792721; or (c) chr7:142801104-142802543.

In some embodiments, an engineered cell, which has reduced or eliminated surface expression of TRBC relative to an unmodified cell, is provided, the engineered cell comprising a genetic modification in the TRBC gene, wherein the genetic modification comprises at least one nucleotide within the genomic coordinates chosen from: (a) chr7:142792690-142792714; or chr7:142792693-142792717; or (b) chr7:142791756-142791780; chr7:142791761-142791785; chr7:142791820-142791844; chr7:142791939-142791963; chr7:142791940-142791964; or chr7:142792004-142792028; or (c) chr7:142801104-142801124; chr7:142802103-142802127; or chr7:142802106-142802130.

In some embodiments, an engineered cell, which has reduced or eliminated surface expression of TRBC relative to an unmodified cell, is provided, the engineered cell comprising a genetic modification in the TRBC gene, wherein the genetic modification comprises an indel, a C to T substitution, or an A to G substitution within the genomic coordinates chosen from: (a) chr7:142792690-142792714; or chr7:142792693-142792717; or (b) chr7:142791756-142791780; chr7:142791761-142791785; chr7:142791820-142791844; chr7:142791939-142791963; chr7:142791940-142791964; or chr7:142792004-142792028; or (c) chr7:142801104-142801124; chr7:142802103-142802127; or chr7:142802106-142802130.

In some embodiments, a method of making an engineered cell, which has reduced or eliminated surface expression of TRBC protein relative to an unmodified cell, is provided, the method comprising contacting a cell with a composition of any of the embodiments provided herein. In some embodiments, the composition comprises a TRBC guide RNA, comprising a guide sequence of any one of: SEQ ID NO: 215, 216, 223, 224, 229, 230, 246, 259, and 260. In some embodiments, the composition comprises a TRBC guide RNA, comprising a guide sequence of any one of: SEQ ID NO: 215, 216, 224, 229, 246, 259, and 260. In some embodiments, the composition comprises a TRBC guide RNA, comprising a guide sequence of any one of SEQ ID NOs: 215, 259, and 260.

In some embodiments, the methods comprise reducing or eliminating surface expression of MHC class II protein on the surface of a cell comprising contacting a cell with a composition comprising a CIITA guide RNA comprising a guide sequence that targets a genomic target comprising at least 10 contiguous nucleotides within the genomic coordinates chosen from: (a) chr16:10877363-10907788 or (b) chr16:10906515-10908136. In some embodiments, the methods further comprise contacting the cell with an RNA-guided DNA binding agent or a nucleic acid encoding an RNA-guided DNA binding agent. In some embodiments, the RNA-guided DNA binding agent is Cas9. In some embodiments, the RNA-guided DNA binding agent is N. meningitidis Cas9. In some embodiments, the CIITA guide RNA is a N. meningitidis Cas9 guide RNA. In some embodiments, the RNA-guided DNA binding agent comprises a deaminase domain. In some embodiments the RNA-guided DNA binding agent comprises an APOBEC3A deaminase (A3A) and an RNA-guided nickase. In some embodiments, the expression of MHC class II protein on the surface of the cell (i.e., engineered cell) is thereby reduced. In some embodiments, the CIITA guide RNA comprises a guide sequence selected from SEQ ID NO: 301, 302, 304-576.

In some embodiments, the methods comprise making an engineered cell, which has reduced or eliminated surface expression of MHC class II protein relative to an unmodified cell, comprising contact the cell with a composition comprising a CIITA guide RNA comprising a guide sequence that targets a genomic target comprising at least 10 contiguous nucleotides within the genomic coordinates chosen from: (a) chr16:10877363-10907788 or (b) chr16:10906515-10908136. In some embodiments, the methods further comprise contacting the cell with an RNA-guided DNA binding agent or a nucleic acid encoding an RNA-guided DNA binding agent. In some embodiments, the RNA-guided DNA binding agent is Cas9. In some embodiments, the RNA-guided DNA binding agent is N. meningitidis Cas9. In some embodiments, the CIITA guide RNA is a N. meningitidis Cas9 guide RNA. In some embodiments, the RNA-guided DNA binding agent comprises a deaminase region. In some embodiments the RNA-guided DNA binding agent comprises an APOBEC3A deaminase (A3A) and an RNA-guided nickase. In some embodiments, the expression of MHC class II protein on the surface of the cell (i.e., engineered cell) is thereby reduced. In some embodiments, the CIITA guide RNA comprises a guide sequence selected from SEQ ID NO: 301, 302, 304-576.

In some embodiments, the methods comprise genetically modifying a cell to reduce or eliminate the surface expression of MHC class II protein comprising contacting the cell with a composition comprising a CIITA guide RNA comprising a guide sequence that targets a genomic target comprising at least 10 contiguous nucleotides within the genomic coordinates chosen from: (a) chr16:10877363-10907788 or (b) chr16:10906515-10908136. In some embodiments, the methods further comprise contacting the cell with an RNA-guided DNA binding agent or a nucleic acid encoding an RNA-guided DNA binding agent. In some embodiments, the RNA-guided DNA binding agent is Cas9. In some embodiments, the RNA-guided DNA binding agent is N. meningitidis Cas9. In some embodiments, the CIITA guide RNA is a N. meningitidis Cas9 guide RNA. In some embodiments, the RNA-guided DNA binding agent comprises a deaminase region. In some embodiments the RNA-guided DNA binding agent comprises an APOBEC3A deaminase (A3A) and an RNA-guided nickase. In some embodiments, the expression of MHC class II protein on the surface of the cell (i.e., engineered cell) is thereby reduced. In some embodiments, the CIITA guide RNA comprises a guide sequence selected from SEQ ID NO: 301, 302, 304-576.

In some embodiments, the methods comprise genetically modifying CIITA comprising contacting a cell with a composition comprising a CIITA guide RNA comprising a guide sequence that targets a genomic target comprising at least 10 contiguous nucleotides within the genomic coordinates chosen from: (a) chr16:10877363-10907788 or (b) chr16:10906515-10908136. In some embodiments, the methods further comprise contacting the cell with an RNA-guided DNA binding agent or a nucleic acid encoding an RNA-guided DNA binding agent. In some embodiments, the RNA-guided DNA binding agent is Cas9. In some embodiments, the RNA-guided DNA binding agent is N. meningitidis Cas9. In some embodiments, the CIITA guide RNA is a N. meningitidis Cas9 guide RNA. In some embodiments, the RNA-guided DNA binding agent comprises a deaminase region. In some embodiments the RNA-guided DNA binding agent comprises an APOBEC3A deaminase (A3A) and an RNA-guided nickase. In some embodiments, the expression of MHC class II protein on the surface of the cell (i.e., engineered cell) is thereby reduced. In some embodiments, the CIITA guide RNA comprises a guide sequence selected from SEQ ID NO: 301, 302, 304-576.

In some embodiments, the methods comprise inducing a DSB or an single stranded break (SSB) in CIITA comprising contacting a cell with a composition comprising a CIITA guide RNA comprising a guide sequence targets a genomic target comprising at least 10 contiguous nucleotides within the genomic coordinates chosen from: (a) chr16:10877363-10907788 or (b) chr16:10906515-10908136. In some embodiments, the methods further comprise contacting the cell with an RNA-guided DNA binding agent or a nucleic acid encoding an RNA-guided DNA binding agent. In some embodiments, the RNA-guided DNA binding agent is Cas9. In some embodiments, the RNA-guided DNA binding agent is S. pyogenes Cas9. In some embodiments, the CIITA guide RNA is a N. meningitidis Cas9 guide RNA. In some embodiments, the RNA-guided DNA binding agent comprises a deaminase region. In some embodiments the RNA-guided DNA binding agent comprises an APOBEC3A deaminase (A3A) and an RNA-guided nickase. In some embodiments, the expression of MHC class II protein on the surface of the cell (i.e., engineered cell) is thereby reduced. In some embodiments, the CIITA guide RNA comprises a guide sequence selected from SEQ ID NO: 301, 302, 304-576.

In some embodiments, the methods comprise reducing expression of the CIITA protein in a cell comprising delivering a composition to a cell comprising contacting a cell with a composition comprising a CIITA guide RNA comprising a guide sequence targets a genomic target comprising at least 10 contiguous nucleotides within the genomic coordinates chosen from: (a) chr16:10877363-10907788 or (b) chr16:10906515-10908136. In some embodiments, the methods further comprise contacting the cell with an RNA-guided DNA binding agent or a nucleic acid encoding an RNA-guided DNA binding agent. In some embodiments, the RNA-guided DNA binding agent is Cas9. In some embodiments, the RNA-guided DNA binding agent is N. meningitidis Cas9. In some embodiments, the CIITA guide RNA is a N. meningitidis Cas9 guide RNA. In some embodiments, the RNA-guided DNA binding agent comprises a deaminase region. In some embodiments the RNA-guided DNA binding agent comprises an APOBEC3A deaminase (A3A) and an RNA-guided nickase. In some embodiments, the expression of MHC class II protein on the surface of the cell (i.e., engineered cell) is thereby reduced. In some embodiments, the CIITA guide RNA comprises a guide sequence selected from SEQ ID NO: 301, 302, 304-576.

In some embodiments, the methods of reducing expression of an MHC class II protein on the surface of a cell comprise contacting a cell with any one or more of the CIITA guide RNAs disclosed herein. In some embodiments, the CIITA guide RNA comprises a guide sequence selected from SEQ ID NO: 301, 302, 304-576.

In some embodiments, compositions are provided comprising a CIITA guide RNA comprising a guide sequence that targets a genomic target comprising at least 10 contiguous nucleotides within the genomic coordinates chosen from: (a) chr16:10877363-10907788 or (b) chr16:10906515-10908136. In some embodiments, the composition further comprises an RNA-guided DNA binding agent or a nucleic acid encoding an RNA-guided DNA binding agent. In some embodiments, the composition comprises an RNA-guided DNA binding agent that is Cas9. In some embodiments, the RNA-guided DNA binding agent is N. meningitidis Cas9. In some embodiments, the CIITA guide RNA is a N. meningitidis Cas9 guide RNA. In some embodiments, the RNA-guided DNA binding agent comprises a deaminase region. In some embodiments the RNA-guided DNA binding agent comprises an APOBEC3A deaminase (A3A) and an RNA-guided nickase. In some embodiments, the CIITA guide RNA comprises a guide sequence selected from SEQ ID NO: 301, 302, 304-576.

In some embodiments, a composition is provided, the composition comprising: a) a CIITA guide RNA (gRNA) comprising i) a guide sequence selected from SEQ ID NOs: 301, 302, 304-576; or ii) at least 19, 20, 21, 22, 23, or 24 contiguous nucleotides of a sequence selected from SEQ ID NOs: 301, 302, 304-576; or iii) a guide sequence at least 95%, 90%, or 85% identical to a sequence selected from SEQ ID NOs: 301, 302, 304-576; or iv) a sequence that comprises 10 contiguous nucleotides±10 nucleotides of a genomic coordinate listed in Table 4; or v) at least 19, 20, 21, 22, 23, or 24 contiguous nucleotides of a sequence from (iv); or vi) a guide sequence that is at least 95%, 90%, or 85% identical to a sequence selected from (v). In some embodiments, the CIITA guide RNA that is a single-guide RNA (sgRNA).

In some embodiments, a method of making an engineered cell, which has reduced or eliminated surface expression of MHC class II protein relative to an unmodified cell, is provided, the method comprising contacting a cell with a composition of any of the embodiments provided herein. In some embodiments, the composition comprises a CIITA guide RNA, comprising a guide sequence of any one of: SEQ ID NOs: 301-302, 320-321, 324, 326, 327, 332, 354, 361, 372, 400, 415, 419-420, 422, 428, 431, 432, 434, 451, 455, 458, 462-464, and 468. In some embodiments, the composition comprises a CIITA guide RNA, comprising a guide sequence of any one of: SEQ ID NOs: 301, 302, 320, 372, 414, 419, 422, and 462-463.

In some embodiments, the composition further comprises a uracil glycosylase inhibitor (UGI). In some embodiments, the composition comprises an RNA-guided DNA binding agent that the RNA-guided DNA binding agent generates a cytosine (C) to thymine (T) conversion with the CIITA genomic target sequence. In some embodiments, the composition comprises an RNA-guided DNA binding agent that generates a adenosine (A) to guanine (G) conversion with the CIITA genomic target sequence.

In some embodiments, an engineered cell produced by the methods described herein is provided. In some embodiments, the engineered cell produced by the methods and compositions described herein is an allogeneic cell. In some embodiments, the methods produce a composition comprising an engineered cell having reduced MHC class II expression. In some embodiments, the methods produce a composition comprising an engineered cell having reduced CIITA protein expression. In some embodiments, the methods produce a composition comprising an engineered cell having reduced CIITA levels in the cell nucleus. In some embodiments, the methods produce a composition comprising an engineered cell that expresses a truncated form of the CIITA protein. In some embodiments, the methods produce a composition comprising an engineered cell that produces no detectable CIITA protein. In some embodiments, the engineered cell has reduced MHC class II expression, reduced CIITA protein, or reduced CIITA levels in the cell nucleus as compared to an unmodified cell. In some embodiments, the engineered cell produced by the methods disclosed herein elicits a reduced response from CD4+ T cells as compared to an unmodified cell as measured in an in vitro cell culture assay containing CD4+ T cells.

In some embodiments, an engineered cell produced by the methods or compositions disclosed herein is provided wherein the cell has reduced or eliminated surface expression of MHC class II protein and wherein the cell comprises a genetic modification comprising at least 5 contiguous nucleotides within the genomic coordinates chosen from: (a) chr16:10877363-10907788 or (b) chr16:10906515-10908136. In some embodiments, an engineered cell produced by the methods or compositions disclosed herein is provided wherein the cell has reduced or eliminated surface expression of MHC class II protein and wherein the cell comprises a genetic modification comprising at least 10 contiguous nucleotides within the genomic coordinates chosen from: (a) chr16:10877363-10907788 or (b) chr16:10906515-10908136. In some embodiments, an engineered cell produced by the methods or compositions disclosed herein is provided wherein the cell has reduced or eliminated surface expression of MHC class II protein and wherein the cell comprises a genetic modification comprising at least one C to T substitution or at least one A to G substitution within the genomic coordinates chosen from: (a) chr16:10877363-10907788 or (b) chr16:10906515-10908136.

In some embodiments, the methods comprise making an engineered cell comprising contact the cell with a composition comprising an AAVS1 guide RNA comprising a guide sequence that targets a genomic target comprising at least 10 contiguous nucleotides within the genomic coordinates chr19:55115151-55116209. In some embodiments, the methods further comprise contacting the cell with an RNA-guided DNA binding agent or a nucleic acid encoding an RNA-guided DNA binding agent. In some embodiments, the RNA-guided DNA binding agent is Cas9. In some embodiments, the RNA-guided DNA binding agent is N. meningitidis Cas9. In some embodiments, the AAVS1 guide RNA is a N. meningitidis Cas9 guide RNA. In some embodiments, the RNA-guided DNA binding agent comprises a deaminase region. In some embodiments the RNA-guided DNA binding agent comprises an APOBEC3A deaminase (A3A) and an RNA-guided nickase. In some embodiments, the AAVS1 guide RNA comprises a guide sequence selected from SEQ ID NO: 601-774.

In some embodiments, the methods comprise genetically modifying a cell comprising contacting the cell with a composition comprising an AAVS1 guide RNA comprising a guide sequence that targets a genomic target comprising at least 10 contiguous nucleotides within the genomic coordinates chr19:55115151-55116209. In some embodiments, the methods further comprise contacting the cell with an RNA-guided DNA binding agent or a nucleic acid encoding an RNA-guided DNA binding agent. In some embodiments, the RNA-guided DNA binding agent is Cas9. In some embodiments, the RNA-guided DNA binding agent is N. meningitidis Cas9. In some embodiments, the AAVS1 guide RNA is a N. meningitidis Cas9 guide RNA. In some embodiments, the RNA-guided DNA binding agent comprises a deaminase region. In some embodiments the RNA-guided DNA binding agent comprises an APOBEC3A deaminase (A3A) and an RNA-guided nickase. In some embodiments, the AAVS1 guide RNA comprises a guide sequence selected from SEQ ID NO: 601-774.

In some embodiments, the methods comprise genetically modifying AAVS1 comprising contacting a cell with a composition comprising an AAVS1 guide RNA comprising a guide sequence that targets a genomic target comprising at least 10 contiguous nucleotides within the genomic coordinates chr19:55115151-55116209. In some embodiments, the methods further comprise contacting the cell with an RNA-guided DNA binding agent or a nucleic acid encoding an RNA-guided DNA binding agent. In some embodiments, the RNA-guided DNA binding agent is Cas9. In some embodiments, the RNA-guided DNA binding agent is N. meningitidis Cas9. In some embodiments, the AAVS1 guide RNA is a N. meningitidis Cas9 guide RNA. In some embodiments, the RNA-guided DNA binding agent comprises a deaminase region. In some embodiments the RNA-guided DNA binding agent comprises an APOBEC3A deaminase (A3A) and an RNA-guided nickase. In some embodiments, the AAVS1 guide RNA comprises a guide sequence selected from SEQ ID NO: 601-774.

In some embodiments, the methods comprise inducing a DSB or a single stranded break (SSB) in AAVS1 comprising contacting a cell with a composition comprising an AAVS1 guide RNA comprising a guide sequence targets a genomic target comprising at least 10 contiguous nucleotides within the genomic coordinates chr19:55115151-55116209. In some embodiments, the methods further comprise contacting the cell with an RNA-guided DNA binding agent or a nucleic acid encoding an RNA-guided DNA binding agent. In some embodiments, the RNA-guided DNA binding agent is Cas9. In some embodiments, the RNA-guided DNA binding agent is S. pyogenes Cas9. In some embodiments, the AAVS1 guide RNA is a N. meningitidis Cas9 guide RNA. In some embodiments, the RNA-guided DNA binding agent comprises a deaminase region. In some embodiments the RNA-guided DNA binding agent comprises an APOBEC3A deaminase (A3A) and an RNA-guided nickase. In some embodiments, the AAVS1 guide RNA comprises a guide sequence selected from SEQ ID NO: 601-774.

In some embodiments, the methods comprise delivering a composition to a cell comprising contacting a cell with a composition comprising an AAVS1 guide RNA comprising a guide sequence targets a genomic target comprising at least 10 contiguous nucleotides within the genomic coordinates chr19:55115151-55116209. In some embodiments, the methods further comprise contacting the cell with an RNA-guided DNA binding agent or a nucleic acid encoding an RNA-guided DNA binding agent. In some embodiments, the RNA-guided DNA binding agent is Cas9. In some embodiments, the RNA-guided DNA binding agent is N. meningitidis Cas9. In some embodiments, the AAVS1 guide RNA is a N. meningitidis Cas9 guide RNA. In some embodiments, the RNA-guided DNA binding agent comprises a deaminase region. In some embodiments the RNA-guided DNA binding agent comprises an APOBEC3A deaminase (A3A) and an RNA-guided nickase. In some embodiments, the AAVS1 guide RNA comprises a guide sequence selected from SEQ ID NO: 601-774.

In some embodiments, the methods of genetically modifying the AAVS1 gene comprise contacting a cell with any one or more of the AAVS1 guide RNAs disclosed herein. In some embodiments, the AAVS1 guide RNA comprises a guide sequence selected from SEQ ID NO: 601-774.

In some embodiments, compositions are provided comprising an AAVS1 guide RNA comprising a guide sequence that targets a genomic target comprising at least 10 contiguous nucleotides within the genomic coordinates chr19:55115151-55116209. In some embodiments, the composition further comprises an RNA-guided DNA binding agent or a nucleic acid encoding an RNA-guided DNA binding agent. In some embodiments, the composition comprises an RNA-guided DNA binding agent that is Cas9. In some embodiments, the RNA-guided DNA binding agent is N. meningitidis Cas9. In some embodiments, the AAVS1 guide RNA is a N. meningitidis Cas9 guide RNA. In some embodiments, the RNA-guided DNA binding agent comprises a deaminase region. In some embodiments the RNA-guided DNA binding agent comprises an APOBEC3A deaminase (A3A) and an RNA-guided nickase. In some embodiments, the AAVS1 guide RNA comprises a guide sequence selected from SEQ ID NO: 601-774.

In some embodiments, a composition is provided, the composition comprising: a) an AAVS1 guide RNA (gRNA) comprising i) a guide sequence selected from SEQ ID NOs: 601-774; or ii) at least 19, 20, 21, 22, 23, or 24 contiguous nucleotides of a sequence selected from SEQ ID NOs: 601-774; or iii) a guide sequence at least 95%, 90%, or 85% identical to a sequence selected from SEQ ID NOs: 601-774; or iv) a sequence that comprises 10 contiguous nucleotides±10 nucleotides of a genomic coordinate listed in Table 5; or v) at least 19, 20, 21, 22, 23, or 24 contiguous nucleotides of a sequence from (iv); or vi) a guide sequence that is at least 95%, 90%, or 85% identical to a sequence selected from (v). In some embodiments, the AAVS1 guide RNA (gRNA) is a single guide RNA.

In some embodiments, a method of making an engineered cell is provided, the method comprising contacting a cell with a composition of any of the embodiments provided herein. In some embodiments, the composition comprises an AAVS1 guide RNA, comprising a guide sequence of any one of from: SEQ ID NOs: 611, 620, 622, 626, 627, 628, 629, 632, 633, 634, 656, 659, 660, 661, 673, 691, 692, 730, 734, and 746.

In some embodiments, the composition further comprises a uracil glycosylase inhibitor (UGI). In some embodiments, the composition comprises an RNA-guided DNA binding agent that the RNA-guided DNA binding agent generates a cytosine (C) to thymine (T) conversion with the AAVS1 genomic target sequence. In some embodiments, the composition comprises an RNA-guided DNA binding agent that generates a adenosine (A) to guanine (G) conversion with the AAVS1 genomic target sequence.

In some embodiments, an engineered cell produced by the methods described herein is provided. In some embodiments, the engineered cell produced by the methods and compositions described herein is an allogeneic cell. In some embodiments, the engineered cell produced by the methods disclosed herein elicits a reduced response from CD4+ T cells as compared to an unmodified cell as measured in an in vitro cell culture assay containing CD4+ T cells.

In some embodiments, an engineered cell produced by the methods or compositions disclosed herein is provided wherein the cell comprises a genetic modification comprising at least 5 contiguous nucleotides within the genomic coordinates chr19:55115151-55116209. In some embodiments, an engineered cell produced by the methods or compositions disclosed herein is provided wherein the cell comprises a genetic modification comprising at least 10 contiguous nucleotides within the genomic coordinates chr19:55115151-55116209. In some embodiments, an engineered cell produced by the methods or compositions disclosed herein is provided wherein the cell comprises a genetic modification comprising at least one C to T substitution or at least one A to G substitution within the genomic coordinates chr19:55115151-55116209. In some embodiments, the compositions disclosed herein further comprise a pharmaceutically acceptable carrier. In some embodiments, a cell produced by the compositions disclosed herein comprising a pharmaceutically acceptable carrier is provided. In some embodiments, compositions comprising the cells disclosed herein are provided.

### C. HLA-A Guide RNAs

The methods and compositions provided herein disclose HLA-A guide RNAs useful for reducing the expression of HLA-A protein on the surface of a cell. In some embodiments, such guide RNAs direct an RNA-guided DNA binding agent to an HLA-A genomic target sequence and may be referred to herein as “HLA-A guide RNAs.” In some embodiments, the HLA-A guide RNA directs an RNA-guided DNA binding agent to a human HLA-A genomic target sequence. In some embodiments, the HLA-A guide RNA comprises a guide sequence selected from SEQ ID NO: 2-80.

In some embodiments, the methods and compositions disclosed herein comprise an HLA-A guide RNA comprising a guide sequence that targets an HLA-A genomic target sequence comprising at least 10 nucleotides within the genomic coordinates chr6:29942540-29945459. In some embodiments, the method and composition disclosed herein comprise an HLA-A guide RNA comprising a guide sequence that targets an HLA-A genomic target sequence comprising at least one nucleotide within the genomic coordinates chr6:29942540-29945459.

In some embodiments, the methods and compositions disclosed herein comprise an HLA-A guide RNA comprising a guide sequence that directs an RNA-guided DNA binding agent to induce a double stranded break (DSB) or a single-stranded break (SSB) in an HLA-A gene, wherein the HLA-A guide RNA targets and HLA-A genomic target sequence comprising at least 10 contiguous nucleotides within the genomic coordinates chr6:29942540-29945459. In some embodiments, the methods and compositions disclosed herein comprise an HLA-A guide RNA comprising a guide sequence that directs an RNA-guided DNA binding agent to induce a double stranded break (DSB) or a single-stranded break (SSB) in an HLA-A gene, wherein the HLA-A guide RNA targets an HLA-A genomic target sequence comprising at least one nucleotide within the genomic coordinates chr6:29942540-29945459.

In some embodiments, the methods and compositions disclose an HLA-A guide RNA that directs an RNA-guided DNA binding agent to induce a double stranded break (DSB) or a single-stranded break (SSB) in an HLA-A genomic target sequence. In some embodiments, the methods and compositions disclose an HLA-A guide RNA that directs an RNA-guided DNA binding agent to make a cut in an HLA-A genomic target sequence. In embodiments wherein the RNA-guided DNA cutting agent is Cas9, the cut occurs at the third base from the protospacer adjacent motif (PAM) sequence.

In some embodiments, a composition is provided comprising an HLA-A guide RNA described herein and an RNA-guided DNA binding agent or a nucleic acid encoding an RNA-guided DNA binding agent.

In some embodiments, a composition is provided comprising an HLA-A single-guide RNA (sgRNA) comprising a guide sequence that targets a genomic target comprising at least 10 contiguous nucleotides within the genomic coordinates chr6:29942540-29945459. In some embodiments, a composition is provided comprising an HLA-A sgRNA described herein and an RNA-guided DNA binding agent or a nucleic acid encoding an RNA-guided DNA binding agent.

In some embodiments, a composition is provided comprising an HLA-A dual-guide RNA (dgRNA) comprising a guide sequence that targets a genomic target comprising at least 10 contiguous nucleotides within the genomic coordinates chr6:29942540-29945459. In some embodiments, a composition is provided comprising an HLA-A dgRNA described herein and an RNA-guided DNA binding agent or a nucleic acid encoding an RNA-guided DNA binding agent.

In some embodiments, the TRAC gRNA comprises a guide sequence selected from any one of SEQ ID NOs: 2-80. Exemplary HLA-A guide sequences are shown below in Table 1 (SEQ ID NOs: 2-80) with corresponding guide RNA sequences 2-80.

TABLE 1
Exemplary HLA-A guide sequences

| | | | Exemplary | | |
|---|---|---|---|---|---|
| | | | Guide RNA | | |
| | SEQ ID | | Full | | |
| | NO to | | Sequence | Exemplary Guide | |
| | the | | (SEQ ID | RNA Modified | Genomic |
| Guide | Guide | Guide | NOs: | Sequence (SEQ ID | Coordinates |
| ID | Sequence | Sequence | 1002-1080) | NOs: 2002-2080) | (hg38) |
| G028854 | 2 | CCUGGGUCUGG | CCUGGGUCUGG | mC*mC*mU*mGmGGUm | chr6: |
| | | UCCUCCCCAUC | UCCUCCCCAUC | CmUGmGUmCCUCCmCC | 29944223-29944247 |
| | | CC | CCGUUGUAGCU | AUmCCCmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G028855 | 3 | GUGGAGACCAG | GUGGAGACCAG | mG*mU*mG*mGmAGAm | chr6: |
| | | GCCUGCAGGGG | GCCUGCAGGGG | CmCAmGGmCCUGCmAG | 29944264-29944288 |
| | | AU | AUGUUGUAGCU | GGmGAUmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G028856 | 4 | CCGUGUCCUGG | CCGUGUCCUGG | mC*mC*mG*mUmGUCm | chr6: |
| | | GUCUGGUCCUC | GUCUGGUCCUC | CmUGmGGmUCUGGmUC | 29944229-29944253 |
| | | CC | CCGUUGUAGCU | CUmCCCmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G028857 | 5 | UCCGUGUCCUG | UCCGUGUCCUG | mU*mC*mC*mGmUGUm | chr6: |
| | | GGUCUGGUCCU | GGUCUGGUCCU | CmCUmGGmGUCUGmGU | 29944230-29944254 |
| | | CC | CCGUUGUAGCU | CCmUCCmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G028858 | 6 | CACAUGGCAGG | CACAUGGCAGG | mC*mA*mC*mAmUGGm | chr6: |
| | | UGUAUCUCUGC | UGUAUCUCUGC | CmAGmGUmGUAUCmUC | 29944327-29944351 |
| | | UC | UCGUUGUAGCU | UGmCUCmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G028859 | 7 | UGCUGCACAUG | UGCUGCACAUG | mU*mG*mC*mUmGCAm | chr6: |
| | | GCAGGUGUAUC | GCAGGUGUAUC | CmAUmGGmCAGGUmGU | 29944332-29944356 |
| | | UC | UCGUUGUAGCU | AUmCUCmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G028860 | 8 | CCGCACGAACU | CCGCACGAACU | mC*mC*mG*mCmACGm | chr6: |
| | | GCGUGUCGUCC | GCGUGUCGUCC | AmACmUGmCGUGUmCG | 29942830-29942854 |
| | | AC | ACGUUGUAGCU | UCmCACmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G028861 | 9 | CGCACGAACUG | CGCACGAACUG | mC*mG*mC*mAmCGAm | chr6: |
| | | CGUGUCGUCCA | CGUGUCGUCCA | AmCUmGCmGUGUCmGU | 29942829-29942853 |
| | | CG | CGGUUGUAGCU | CCmACGmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G028862 | 10 | GUGCGCUGCAG | GUGCGCUGCAG | mG*mU*mG*mCmGCUm | chr6: |
| | | CGUCUCCUUCC | CGUCUCCUUCC | GmCAmGCmGUCUCmCU | 29943511-29943535 |
| | | CG | CGGUUGUAGCU | UCmCCGmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G028863 | 11 | CCGAGGAUGGC | CCGAGGAUGGC | mC*mC*mG*mAmGGAm | chr6: |
| | | CGUCAUGGCGC | CGUCAUGGCGC | UmGGmCCmGUCAUmGG | 29942547-29942571 |
| | | CC | CCGUUGUAGCU | CGmCCCmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G028864 | 12 | CGAGGAUGGCC | CGAGGAUGGCC | mC*mG*mA*mGmGAUm | chr6: |
| | | GUCAUGGCGCC | GUCAUGGCGCC | GmGCmCGmUCAUGmGC | 29942548-29942572 |
| | | CC | CCGUUGUAGCU | GCmCCCmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G028865 | 13 | AAGGUUCCAUC | AAGGUUCCAUC | mA*mA*mG*mGmUUCm | chr6: |
| | | CCCUGCAGGCC | CCCUGCAGGCC | CmAUmCCmCCUGCmAG | 29944266-29944290 |
| | | UG | UGGUUGUAGCU | GCmCUGmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G028866 | 14 | AGACCAGGCCU | AGACCAGGCCU | mA*mG*mA*mCmCAGm | chr6: |
| | | GCAGGGGAUGG | GCAGGGGAUGG | GmCCmUGmCAGGGmGA | 29944268-29944292 |
| | | AA | AAGUUGUAGCU | UGmGAAmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G028867 | 15 | ACUUCUGGAAG | ACUUCUGGAAG | mA*mC*mU*mUmCUGm | chr6: |
| | | GUUCCAUCCCC | GUUCCAUCCCC | GmAAmGGmUUCCAmUC | 29944274-29944298 |
| | | UG | UGGUUGUAGCU | CCmCUGmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G028868 | 16 | CGAUGAAGCGG | CGAUGAAGCGG | mC*mG*mA*mUmGAAm | chr6: |
| | | GGCUCCCCGCG | GGCUCCCCGCG | GmCGmGGmGCUCCmCC | 29942795-29942819 |
| | | GC | GCGUUGUAGCU | GCmGGCmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G028869 | 17 | UCCGUGUCCCG | UCCGUGUCCCG | mU*mC*mC*mGmUGUm | chr6: |
| | | GCCCGGCCGCG | GCCCGGCCGCG | CmCCmGGmCCCGGmCC | 29942785-29942809 |
| | | GG | GGGUUGUAGCU | GCmGGGmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G028870 | 18 | CCGUGUCCCGG | CCGUGUCCCGG | mC*mC*mG*mUmGUCm | chr6: |
| | | CCCGGCCGCGG | CCCGGCCGCGG | CmCGmGCmCCGGCmCG | 29942786-29942810 |
| | | GG | GGGUUGUAGCU | CGmGGGmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G028871 | 19 | CAGACGCCGAG | CAGACGCCGAG | mC*mA*mG*mAmCGCm | chr6: |
| | | GAUGGCCGUCA | GAUGGCCGUCA | CmGAmGGmAUGGCmCG | 29942541-29942565 |
| | | UG | UGGUUGUAGCU | UCmAUGmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G028872 | 20 | CCAGACGCCGA | CCAGACGCCGA | mC*mC*mA*mGmACGm | chr6: |
| | | GGAUGGCCGUC | GGAUGGCCGUC | CmCGmAGmGAUGGmCC | 29942540-29942564 |
| | | AU | AUGUUGUAGCU | GUmCAUmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G028873 | 21 | AGACGCCGAGG | AGACGCCGAGG | mA*mG*mA*mCmGCCm | chr6: |
| | | AUGGCCGUCAU | AUGGCCGUCAU | GmAGmGAmUGGCCmGU | 29942542-29942566 |
| | | GG | GGGUUGUAGCU | CAmUGGmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G028874 | 22 | CUGUCGAACCG | CUGUCGAACCG | mC*mU*mG*mUmCGAm | chr6: |
| | | CACGAACUGCG | CACGAACUGCG | AmCCmGCmACGAAmCU | 29942838-29942862 |
| | | UG | UGGUUGUAGCU | GCmGUGmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G028875 | 23 | UCGUGCGGUUC | UCGUGCGGUUC | mU*mC*mG*mUmGCGm | chr6: |
| | | GACAGCGACGC | GACAGCGACGC | GmUUmCGmACAGCmGA | 29942852-29942876 |
| | | CG | CGGUUGUAGCU | CGmCCGmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G028876 | 24 | CCAUCCCCAUC | CCAUCCCCAUC | mC*mC*mA*mUmCCCm | chr6: |
| | | GUGGGCAUCAU | GUGGGCAUCAU | CmAUmCGmUGGGCmAU | 29944517-29944541 |
| | | UG | UGGUUGUAGCU | CAmUUGmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G028877 | 25 | UCCUCUGGCUC | UCCUCUGGCUC | mU*mC*mC*mUmCUGm | chr6: |
| | | GCGGCGUCGCU | GCGGCGUCGCU | GmCUmCGmCGGCGmUC | 29942858-29942882 |
| | | GU | GUGUUGUAGCU | GCmUGUmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G028878 | 26 | CUCUGGUUGUA | CUCUGGUUGUA | mC*mU*mC*mUmGGUm | chr6: |
| | | GUAGCCGCGCA | GUAGCCGCGCA | UmGUmAGmUAGCCmGC | 29942988-29943012 |
| | | GG | GGGUUGUAGCU | GCmAGGmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G028879 | 27 | ACCUGGGGACC | ACCUGGGGACC | mA*mC*mC*mUmGGGm | chr6: |
| | | CUGCGCGGCUA | CUGCGCGGCUA | GmACmCCmUGCGCmGG | 29942984-29943008 |
| | | CU | CUGUUGUAGCU | CUmACUmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G028880 | 28 | CGCUCUGGUUG | CGCUCUGGUUG | mC*mG*mC*mUmCUGm | chr6: |
| | | UAGUAGCCGCG | UAGUAGCCGCG | GmUUmGUmAGUAGmCC | 29942990-29943014 |
| | | CA | CAGUUGUAGCU | GCmGCAmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G028881 | 29 | GCUCUGGUUGU | GCUCUGGUUGU | mG*mC*mU*mCmUGGm | chr6: |
| | | AGUAGCCGCGC | AGUAGCCGCGC | UmUGmUAmGUAGCmCG | 29942989-29943013 |
| | | AG | AGGUUGUAGCU | CGmCAGmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G028882 | 30 | UAUUUUUUUCU | UAUUUUUUUCU | mU*mA*mU*mUmUUUm | chr6: |
| | | AUAGUGUGAGA | AUAGUGUGAGA | UmUCmUAmUAGUGmUG | 29945435-29945459 |
| | | CA | CAGUUGUAGCU | AGmACAmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G028883 | 31 | ACGCCUACGAC | ACGCCUACGAC | mA*mC*mG*mCmCUAm | chr6: |
| | | GGCAAGGAUUA | GGCAAGGAUUA | CmGAmCGmGCAAGmGA | 29943342-29943366 |
| | | CA | CAGUUGUAGCU | UUmACAmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G028884 | 32 | UUUGUUCUACC | UUUGUUCUACC | mU*mU*mU*mGmUUCm | chr6: |
| | | CCAGGCAGUGA | CCAGGCAGUGA | UmACmCCmCAGGCmAG | 29945218-29945242 |
| | | CA | CAGUUGUAGCU | UGmACAmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G028885 | 33 | UUUUGUUCUAC | UUUUGUUCUAC | mU*mU*mU*mUmGUUm | chr6: |
| | | CCCAGGCAGUG | CCCAGGCAGUG | CmUAmCCmCCAGGmCA | 29945217-29945241 |
| | | AC | ACGUUGUAGCU | GUmGACmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G028886 | 34 | AGCGACCACAG | AGCGACCACAG | mA*mG*mC*mGmACCm | chr6: |
| | | CUCCAGUGAUC | CUCCAGUGAUC | AmCAmGCmUCCAGmUG | 29944558-29944582 |
| | | AC | ACGUUGUAGCU | AUmCACmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G028887 | 35 | GAGCUCCGUGU | GAGCUCCGUGU | mG*mA*mG*mCmUCCm | chr6: |
| | | CCUGGGUCUGG | CCUGGGUCUGG | GmUGmUCmCUGGGmUC | 29944234-29944258 |
| | | UC | UCGUUGUAGCU | UGmGUCmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G028888 | 36 | UCCACGAGCUC | UCCACGAGCUC | mU*mC*mC*mAmCGAm | chr6: |
| | | CGUGUCCUGGG | CGUGUCCUGGG | GmCUmCCmGUGUCmCU | 29944239-29944263 |
| | | UC | UCGUUGUAGCU | GGmGUCmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G028889 | 37 | ACGAGCUCCGU | ACGAGCUCCGU | mA*mC*mG*mAmGCUm | chr6: |
| | | GUCCUGGGUCU | GUCCUGGGUCU | CmCGmUGmUCCUGmGG | 29944236-29944260 |
| | | GG | GGGUUGUAGCU | UCmUGGmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G028890 | 38 | CGAGCUCCGUG | CGAGCUCCGUG | mC*mG*mA*mGmCUCm | chr6: |
| | | UCCUGGGUCUG | UCCUGGGUCUG | CmGUmGUmCCUGGmGU | 29944235-29944259 |
| | | GU | GUGUUGUAGCU | CUmGGUmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G028891 | 39 | GCAGGCCUGGU | GCAGGCCUGGU | mG*mC*mA*mGmGCCm | chr6: |
| | | CUCCACGAGCU | CUCCACGAGCU | UmGGmUCmUCCACmGA | 29944251-29944275 |
| | | CC | CCGUUGUAGCU | GCmUCCmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G028892 | 40 | UCCCCUGCAGG | UCCCCUGCAGG | mU*mC*mC*mCmCUGm | chr6: |
| | | CCUGGUCUCCA | CCUGGUCUCCA | CmAGmGCmCUGGUmCU | 29944257-29944281 |
| | | CG | CGGUUGUAGCU | CCmACGmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G028893 | 41 | AUCCGUGUCCC | AUCCGUGUCCC | mA*mU*mC*mCmGUGm | chr6: |
| | | GGCCCGGCCGC | GGCCCGGCCGC | UmCCmCGmGCCCGmGC | 29942784-29942808 |
| | | GG | GGGUUGUAGCU | CGmCGGmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G028894 | 42 | CCUUCCCGUUC | CCUUCCCGUUC | mC*mC*mU*mUmCCCm | chr6: |
| | | UCCAGGUAUCU | UCCAGGUAUCU | GmUUmCUmCCAGGmUA | 29943495-29943519 |
| | | GC | GCGUUGUAGCU | UCmUGCmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G028895 | 43 | CCGUUCUCCAG | CCGUUCUCCAG | mC*mC*mG*mUmUCUm | chr6: |
| | | GUAUCUGCGGA | GUAUCUGCGGA | CmCAmGGmUAUCUmGC | 29943490-29943514 |
| | | GC | GCGUUGUAGCU | GGmAGCmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G028896 | 44 | ACCUGCCAUGU | ACCUGCCAUGU | mA*mC*mC*mUmGCCm | chr6: |
| | | GCAGCAUGAGG | GCAGCAUGAGG | AmUGmUGmCAGCAmUG | 29944345-29944369 |
| | | GU | GUGUUGUAGCU | AGmGGUmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G028897 | 45 | CACCUGCCAUG | CACCUGCCAUG | mC*mA*mC*mCmUGCm | chr6: |
| | | UGCAGCAUGAG | UGCAGCAUGAG | CmAUmGUmGCAGCmAU | 29944344-29944368 |
| | | GG | GGGUUGUAGCU | GAmGGGmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G028898 | 46 | ACCUGGCAGCG | ACCUGGCAGCG | mA*mC*mC*mUmGGCm | chr6: |
| | | GGAUGGGGAGG | GGAUGGGGAGG | AmGCmGGmGAUGGmGG | 29944219-29944243 |
| | | AC | ACGUUGUAGCU | AGmGACmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G028899 | 47 | CACUGACCUGG | CACUGACCUGG | mC*mA*mC*mUmGACm | chr6: |
| | | CAGCGGGAUGG | CAGCGGGAUGG | CmUGmGCmAGCGGmGA | 29944214-29944238 |
| | | GG | GGGUUGUAGCU | UGmGGGmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G028900 | 48 | CCUGGCAGCGG | CCUGGCAGCGG | mC*mC*mU*mGmGCAm | chr6: |
| | | GAUGGGGAGGA | GAUGGGGAGGA | GmCGmGGmAUGGGmGA | 29944220-29944244 |
| | | CC | CCGUUGUAGCU | GGmACCmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G028901 | 49 | CCAUCUCAGGG | CCAUCUCAGGG | mC*mC*mA*mUmCUCm | chr6: |
| | | UGAGGGGCUUG | UGAGGGGCUUG | AmGGmGUmGAGGGmGC | 29944366-29944390 |
| | | GG | GGGUUGUAGCU | UUmGGGmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G028902 | 50 | UGGCCGUCAUG | UGGCCGUCAUG | mU*mG*mG*mCmCGUm | chr6: |
| | | GCGCCCCGAAC | GCGCCCCGAAC | CmAUmGGmCGCCCmCG | 29942554-29942578 |
| | | CC | CCGUUGUAGCU | AAmCCCmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G028903 | 51 | AUGUCCGCCGC | AUGUCCGCCGC | mA*mU*mG*mUmCCGm | chr6: |
| | | GGUCCAAGAGC | GGUCCAAGAGC | CmCGmCGmGUCCAmAG | 29943379-29943403 |
| | | GC | GCGUUGUAGCU | AGmCGCmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G028904 | 52 | GGUACCCGUGC | GGUACCCGUGC | mG*mG*mU*mAmCCCm | chr6: |
| | | GCUGCAGCGUC | GCUGCAGCGUC | GmUGmCGmCUGCAmGC | 29943518-29943542 |
| | | UC | UCGUUGUAGCU | GUmCUCmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G028905 | 53 | GGGAAGGAGAC | GGGAAGGAGAC | mG*mG*mG*mAmAGGm | chr6: |
| | | GCUGCAGCGCA | GCUGCAGCGCA | AmGAmCGmCUGCAmGC | 29943518-29943542 |
| | | CG | CGGUUGUAGCU | GCmACGmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G028906 | 54 | GCGGGCGCCGU | GCGGGCGCCGU | mG*mC*mG*mGmGCGm | chr6: |
| | | GGAUAGAGCAG | GGAUAGAGCAG | CmCGmUGmGAUAGmAG | 29942895-29942919 |
| | | GA | GAGUUGUAGCU | CAmGGAmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G028907 | 55 | UCCUGCUCUAU | UCCUGCUCUAU | mU*mC*mC*mUmGCUm | chr6: |
| | | CCACGGCGCCC | CCACGGCGCCC | CmUAmUCmCACGGmCG | 29942889-29942913 |
| | | GC | GCGUUGUAGCU | CCmCGCmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G028908 | 56 | CCCCUGGUACC | CCCCUGGUACC | mC*mC*mC*mCmUGGm | chr6: |
| | | CGUGCGCUGCA | CGUGCGCUGCA | UmACmCCmGUGCGmCU | 29943523-29943547 |
| | | GC | GCGUUGUAGCU | GCmAGCmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G028909 | 57 | UGGUACCCGUG | UGGUACCCGUG | mU*mG*mG*mUmACCm | chr6: |
| | | CGCUGCAGCGU | CGCUGCAGCGU | CmGUmGCmGCUGCmAG | 29943519-29943543 |
| | | CU | CUGUUGUAGCU | CGmUCUmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G028910 | 58 | CAUCUCAGGGU | CAUCUCAGGGU | mC*mA*mU*mCmUCAm | chr6: |
| | | GAGGGGCUUGG | GAGGGGCUUGG | GmGGmUGmAGGGGmCU | 29944365-29944389 |
| | | GC | GCGUUGUAGCU | UGmGGCmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G028911 | 59 | ACGCAGUUCGU | ACGCAGUUCGU | mA*mC*mG*mCmAGUm | chr6: |
| | | GCGGUUCGACA | GCGGUUCGACA | UmCGmUGmCGGUUmCG | 29942845-29942869 |
| | | GC | GCGUUGUAGCU | ACmAGCmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G028912 | 60 | CGCCCAGGUCU | CGCCCAGGUCU | mC*mG*mC*mCmCAGm | chr6: |
| | | GGGUCAGGGCC | GGGUCAGGGCC | GmUCmUGmGGUCAmGG | 29942595-29942619 |
| | | AG | AGGUUGUAGCU | GCmCAGmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G028913 | 61 | CACUCACCCGC | CACUCACCCGC | mC*mA*mC*mUmCACm | chr6: |
| | | CCAGGUCUGGG | CCAGGUCUGGG | CmCGmCCmCAGGUmCU | 29942609-29942633 |
| | | UC | UCGUUGUAGCU | GGmGUCmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G028914 | 62 | CCGCCCAGGUC | CCGCCCAGGUC | mC*mC*mG*mCmCCAm | chr6: |
| | | UGGGUCAGGGC | UGGGUCAGGGC | GmGUmCUmGGGUCmAG | 29942596-29942620 |
| | | CA | CAGUUGUAGCU | GGmCCAmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G028915 | 63 | CCCGCCCAGGU | CCCGCCCAGGU | mC*mC*mC*mGmCCCm | chr6: |
| | | CUGGGUCAGGG | CUGGGUCAGGG | AmGGmUCmUGGGUmCA | 29942597-29942621 |
| | | CC | CCGUUGUAGCU | GGmGCCmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G028916 | 64 | CAGCGACGCCG | CAGCGACGCCG | mC*mA*mG*mCmGACm | chr6: |
| | | CGAGCCAGAGG | CGAGCCAGAGG | GmCCmGCmGAGCCmAG | 29942865-29942889 |
| | | AU | AUGUUGUAGCU | AGmGAUmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G028917 | 65 | CGCGAGCCAGA | CGCGAGCCAGA | mC*mG*mC*mGmAGCm | chr6: |
| | | GGAUGGAGCCG | GGAUGGAGCCG | CmAGmAGmGAUGGmAG | 29942874-29942898 |
| | | CG | CGGUUGUAGCU | CCmGCGmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G028918 | 66 | GCUCUAUCCAC | GCUCUAUCCAC | mG*mC*mU*mCmUAUm | chr6: |
| | | GGCGCCCGCGG | GGCGCCCGCGG | CmCAmCGmGCGCCmCG | 29942891-29942915 |
| | | CU | CUGUUGUAGCU | CGmGCUmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G028919 | 67 | CACAUCAGAGC | CACAUCAGAGC | mC*mA*mC*mAmUCAm | chr6: |
| | | CCUGGGCACUG | CCUGGGCACUG | GmAGmCCmCUGGGmCA | 29945232-29945256 |
| | | UC | UCGUUGUAGCU | CUmGUCmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G028920 | 68 | CAGACCCAGGA | CAGACCCAGGA | mC*mA*mG*mAmCCCm | chr6: |
| | | CACGGAGCUCG | CACGGAGCUCG | AmGGmACmACGGAmGC | 29944243-29944267 |
| | | UG | UGGUUGUAGCU | UCmGUGmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G028921 | 69 | CCAGGACACGG | CCAGGACACGG | mC*mC*mA*mGmGACm | chr6: |
| | | AGCUCGUGGAG | AGCUCGUGGAG | AmCGmGAmGCUCGmUG | 29944248-29944272 |
| | | AC | ACGUUGUAGCU | GAmGACmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G028922 | 70 | CUCUGGGAAAA | CUCUGGGAAAA | mC*mU*mC*mUmGGGm | chr6: |
| | | GAGGGGAAGGU | GAGGGGAAGGU | AmAAmAGmAGGGGmAA | 29944471-29944495 |
| | | GA | GAGUUGUAGCU | GGmUGAmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G028923 | 71 | UCUGGGAAAAG | UCUGGGAAAAG | mU*mC*mU*mGmGGAm | chr6: |
| | | AGGGGAAGGUG | AGGGGAAGGUG | AmAAmGAmGGGGAmAG | 29944470-29944494 |
| | | AG | AGGUUGUAGCU | GUmGAGmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G028924 | 72 | AGACGCUGCAG | AGACGCUGCAG | mA*mG*mA*mCmGCUm | chr6: |
| | | CGCACGGGUAC | CGCACGGGUAC | GmCAmGCmGCACGmGG | 29943525-29943549 |
| | | CA | CAGUUGUAGCU | UAmCCAmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G028925 | 73 | UCCUUUUCUAU | UCCUUUUCUAU | mU*mC*mC*mUmUUUm | Unavailable. |
| | | CUGUGGGAAGA | CUGUGGGAAGA | CmUAmUCmUGUGGmGA | Imperfect |
| | | AA | AAGUUGUAGCU | AGmAAAmGUUGmUmAm | alignment to |
| | | | CCCUGAAACCG | GmCUCCCmUmGmAmAm | human genome. |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G028926 | 74 | CACUGCCUGGG | CACUGCCUGGG | mC*mA*mC*mUmGCCm | chr6: |
| | | GUAGAACAAAA | GUAGAACAAAA | UmGGmGGmUAGAAmCA | 29945209-29945233 |
| | | AC | ACGUUGUAGCU | AAmAACmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G028927 | 75 | ACAACCAGAGC | ACAACCAGAGC | mA*mC*mA*mAmCCAm | chr6: |
| | | GAGGCCGGUGA | GAGGCCGGUGA | GmAGmCGmAGGCCmGG | 29943008-29943032 |
| | | GU | GUGUUGUAGCU | UGmAGUmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G028928 | 76 | CUACAACCAGA | CUACAACCAGA | mC*mU*mA*mCmAACm | chr6: |
| | | GCGAGGCCGGU | GCGAGGCCGGU | CmAGmAGmCGAGGmCC | 29943006-29943030 |
| | | GA | GAGUUGUAGCU | GGmUGAmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G028929 | 77 | UACAACCAGAG | UACAACCAGAG | mU*mA*mC*mAmACCm | chr6: |
| | | CGAGGCCGGUG | CGAGGCCGGUG | AmGAmGCmGAGGCmCG | 29943007-29943031 |
| | | AG | AGGUUGUAGCU | GUmGAGmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G028930 | 78 | CCAGAGCGAGG | CCAGAGCGAGG | mC*mC*mA*mGmAGCm | chr6: |
| | | CCGGUGAGUGA | CCGGUGAGUGA | GmAGmGCmCGGUGmAG | 29943012-29943036 |
| | | CC | CCGUUGUAGCU | UGmACCmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G028933 | 79 | CCAUCCCGCUG | CCAUCCCGCUG | mC*mC*mA*mUmCCCm | chr6: |
| | | CCAGGUCAGUG | CCAGGUCAGUG | GmCUmGCmCAGGUmCA | 29944206-29944230 |
| | | UG | UGGUUGUAGCU | GUmGUGmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G028934 | 80 | GUAUCUGCGGA | GUAUCUGCGGA | mG*mU*mA*mUmCUGm | chr6: |
| | | GCCACUCCACG | GCCACUCCACG | CmGGmAGmCCACUmCC | 29943479-29943503 |
| | | CA | CAGUUGUAGCU | ACmGCAmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |

Throughout this application, the terms “mA,” “mC,” “mU,” or “mG” may be used to denote a nucleotide that has been modified with 2′—O-Me. Throughout this application, the terms A*, C*, U*, or G* may be used to denote a nucleotide that is linked to the next (e.g., 3′) nucleotide with a phosphorothioate (PS) bond.

In some embodiments, the HLA-A guide RNA comprises a guide sequence selected from SEQ ID NOs: 2-80. In some embodiments, the HLA-A guide RNA comprises a guide sequence that is at least 19, 20, 21, 22, 23, or 24 contiguous nucleotides of a sequence selected from SEQ ID NOs: 2-80. In some embodiments, the HLA-A guide RNA comprises a guide sequence that is at least 9500, 90%, or 85% identical to a sequence selected from SEQ ID NOs: 2-80. In some embodiments, the HLA-A guide RNA comprises a guide sequence that is at least 9500 identical to a sequence selected from SEQ ID NOs: 2-80.

In some embodiments, the HLA-A guide RNA comprises a guide sequence that comprises at least 10 contiguous nucleotides±10 nucleotides of a genomic coordinate listed in Table 1. As used herein, at least 10 contiguous nucleotides±10 nucleotides of a genomic coordinate means, for example, at least 10 contiguous nucleotides within the genomic coordinates wherein the genomic coordinates include 10 nucleotides in the 5′ direction and 10 nucleotides in the 3′ direction from the ranges listed in Table 1. For example, an HLA-A guide RNA may comprise 10 contiguous nucleotides within the genomic coordinates chosen from: chr6:29942891-29942915; chr6:29942609-29942633; chr6:29944266-29944290; chr6:29942889-29942913; chr6:29944471-29944495; and chr6:29944470-29944494; including the boundary nucleotides of these ranges. In some embodiments, the HLA-A guide RNA comprises a guide sequence that is at least 19, 20, 21, 22, 23, or 24 contiguous nucleotides of a sequence that comprises 10 contiguous nucleotides±10 nucleotides of a genomic coordinate listed in Table 1. In some embodiments, the HLA-A guide RNA comprises a guide sequence that is at least 95%, 90%, or 85% identical to a sequence selected from a sequence that is 19, 20, 21, 22, 23, or 24 contiguous nucleotides of a sequence that comprises 10 contiguous nucleotides±10 nucleotides of a genomic coordinate listed in Table 1.

In some embodiments, the HLA-A guide RNA comprises a guide sequence that comprises at least 15 contiguous nucleotides±10 nucleotides of a genomic coordinate listed in Table 1. In some embodiments, the HLA-A guide RNA comprises a guide sequence that comprises at least 24 contiguous nucleotides±10 nucleotides of a genomic coordinate listed in Table 1.

In some embodiments, the HLA-A guide RNA comprises SEQ ID NO: 2. In some embodiments, the HLA-A guide RNA comprises SEQ ID NO: 3. In some embodiments, the HLA-A guide RNA comprises SEQ ID NO: 4. In some embodiments, the HLA-A guide RNA comprises SEQ ID NO: 5. In some embodiments, the HLA-A guide RNA comprises SEQ ID NO: 6. In some embodiments, the HLA-A guide RNA comprises SEQ ID NO: 7. In some embodiments, the HLA-A guide RNA comprises SEQ ID NO: 8. In some embodiments, the HLA-A guide RNA comprises SEQ ID NO: 9. In some embodiments, the HLA-A guide RNA comprises SEQ ID NO: 10. In some embodiments, the HLA-A guide RNA comprises SEQ ID NO: 11. In some embodiments, the HLA-A guide RNA comprises SEQ ID NO: 12. In some embodiments, the HLA-A guide RNA comprises SEQ ID NO: 13. In some embodiments, the HLA-A guide RNA comprises SEQ ID NO: 14. In some embodiments, the HLA-A guide RNA comprises SEQ ID NO: 15. In some embodiments, the HLA-A guide RNA comprises SEQ ID NO: 16. In some embodiments, the HLA-A guide RNA comprises SEQ ID NO: 17. In some embodiments, the HLA-A guide RNA comprises SEQ ID NO: 18. In some embodiments, the HLA-A guide RNA comprises SEQ ID NO: 19. In some embodiments, the HLA-A guide RNA comprises SEQ ID NO: 20. In some embodiments, the HLA-A guide RNA comprises SEQ ID NO: 21. In some embodiments, the HLA-A guide RNA comprises SEQ ID NO: 22. In some embodiments, the HLA-A guide RNA comprises SEQ ID NO: 23. In some embodiments, the HLA-A guide RNA comprises SEQ ID NO: 24. In some embodiments, the HLA-A guide RNA comprises SEQ ID NO: 25. In some embodiments, the HLA-A guide RNA comprises SEQ ID NO: 26. In some embodiments, the HLA-A guide RNA comprises SEQ ID NO: 27. In some embodiments, the HLA-A guide RNA comprises SEQ ID NO: 28. In some embodiments, the HLA-A guide RNA comprises SEQ ID NO: 29. In some embodiments, the HLA-A guide RNA comprises SEQ ID NO: 30. In some embodiments, the HLA-A guide RNA comprises SEQ ID NO: 31. In some embodiments, the HLA-A guide RNA comprises SEQ ID NO: 32. In some embodiments, the HLA-A guide RNA comprises SEQ ID NO: 33. In some embodiments, the HLA-A guide RNA comprises SEQ ID NO: 34. In some embodiments, the HLA-A guide RNA comprises SEQ ID NO: 35. In some embodiments, the HLA-A guide RNA comprises SEQ ID NO: 36. In some embodiments, the HLA-A guide RNA comprises SEQ ID NO: 37. In some embodiments, the HLA-A guide RNA comprises SEQ ID NO: 38. In some embodiments, the HLA-A guide RNA comprises SEQ ID NO: 39. In some embodiments, the HLA-A guide RNA comprises SEQ ID NO: 40. In some embodiments, the HLA-A guide RNA comprises SEQ ID NO: 41. In some embodiments, the HLA-A guide RNA comprises SEQ ID NO: 42. In some embodiments, the HLA-A guide RNA comprises SEQ ID NO: 43. In some embodiments, the HLA-A guide RNA comprises SEQ ID NO: 44. In some embodiments, the HLA-A guide RNA comprises SEQ ID NO: 45. In some embodiments, the HLA-A guide RNA comprises SEQ ID NO: 46. In some embodiments, the HLA-A guide RNA comprises SEQ ID NO: 47. In some embodiments, the HLA-A guide RNA comprises SEQ ID NO: 48. In some embodiments, the HLA-A guide RNA comprises SEQ ID NO: 49. In some embodiments, the HLA-A guide RNA comprises SEQ ID NO: 50. In some embodiments, the HLA-A guide RNA comprises SEQ ID NO: 51. In some embodiments, the HLA-A guide RNA comprises SEQ ID NO: 52. In some embodiments, the HLA-A guide RNA comprises SEQ ID NO: 53. In some embodiments, the HLA-A guide RNA comprises SEQ ID NO: 54. In some embodiments, the HLA-A guide RNA comprises SEQ ID NO: 55. In some embodiments, the HLA-A guide RNA comprises SEQ ID NO: 56. In some embodiments, the HLA-A guide RNA comprises SEQ ID NO: 57. In some embodiments, the HLA-A guide RNA comprises SEQ ID NO: 58. In some embodiments, the HLA-A guide RNA comprises SEQ ID NO: 59. In some embodiments, the HLA-A guide RNA comprises SEQ ID NO: 60. In some embodiments, the HLA-A guide RNA comprises SEQ ID NO: 61. In some embodiments, the HLA-A guide RNA comprises SEQ ID NO: 62. In some embodiments, the HLA-A guide RNA comprises SEQ ID NO: 63. In some embodiments, the HLA-A guide RNA comprises SEQ ID NO: 64. In some embodiments, the HLA-A guide RNA comprises SEQ ID NO: 65. In some embodiments, the HLA-A guide RNA comprises SEQ ID NO: 66. In some embodiments, the HLA-A guide RNA comprises SEQ ID NO: 67. In some embodiments, the HLA-A guide RNA comprises SEQ ID NO: 68. In some embodiments, the HLA-A guide RNA comprises SEQ ID NO: 69. In some embodiments, the HLA-A guide RNA comprises SEQ ID NO: 70. In some embodiments, the HLA-A guide RNA comprises SEQ ID NO: 71. In some embodiments, the HLA-A guide RNA comprises SEQ ID NO: 72. In some embodiments, the HLA-A guide RNA comprises SEQ ID NO: 73. In some embodiments, the HLA-A guide RNA comprises SEQ ID NO: 74. In some embodiments, the HLA-A guide RNA comprises SEQ ID NO: 75. In some embodiments, the HLA-A guide RNA comprises SEQ ID NO: 76. In some embodiments, the HLA-A guide RNA comprises SEQ ID NO: 77. In some embodiments, the HLA-A guide RNA comprises SEQ ID NO: 78. In some embodiments, the HLA-A guide RNA comprises SEQ ID NO: 79. In some embodiments, the HLA-A guide RNA comprises SEQ ID NO: 80.

In some embodiments, the HLA-A guide RNA comprises a guide sequence of any one of: SEQ ID NOs: 13, 55, 61, 66, and 70-71.

In some embodiments, the HLA-A guide RNA comprises a sequence listed in Table 1. In some embodiments, the HLA-A guide RNA comprises a sequence of any one of SEQ ID NO: 2-80. In some embodiments, the HLA-A guide RNA comprises a sequence of SEQ ID NO: 61 or 66. In some embodiments, the HLA-A guide RNA comprises a sequence of SEQ ID NO: 61. In some embodiments, the HLA-A guide RNA comprises a sequence of SEQ ID NO: 61. In some embodiments, the HLA-A guide RNA comprises a guide sequence comprising a sequence of any one of SEQ ID NO: 2-80. In some embodiments, the HLA-A guide RNA comprises a guide sequence comprising a sequence of SEQ ID NO: 61 or 66. In some embodiments, the HLA-A guide RNA comprises a guide sequence comprising a sequence of SEQ ID NO: 61. In some embodiments, the HLA-A guide RNA comprises a guide sequence comprising a sequence of SEQ ID NO: 66. In some embodiments, the HLA-A guide RNA comprises a sequence of any one of SEQ ID NOs: 1002-1080. In some embodiments, the HLA-A guide RNA comprises a sequence of SEQ ID NO: 1061 or 1066. In some embodiments, the HLA-A guide RNA comprises a sequence of SEQ ID NO: 1061. In some embodiments, the HLA-A guide RNA comprises a sequence of SEQ ID NO: 1066. In some embodiments, the HLA-A guide RNA comprises a sequence of any one of SEQ ID NOs: 2002-2080, 3001, and 3002. In some embodiments, the HLA-A guide RNA comprises a sequence of any one of SEQ ID NOs: 2061, 2066, 3001, and 3002. In some embodiments, the HLA-A guide RNA comprises a sequence of SEQ ID NO: 2061. In some embodiments, the HLA-A guide RNA comprises a sequence of SEQ ID NO: 2066. In some embodiments, the HLA-A guide RNA comprises a sequence of SEQ ID NO: 3001. In some embodiments, the HLA-A guide RNA comprises a sequence of SEQ ID NO: 3002.

In some embodiments, the HLA-A guide RNA is a single guide RNA (sgRNA) comprising a sequence of any one of the sgRNA sequences listed in Table 1.

Additional embodiments of HLA-A guide RNAs are provided herein, including e.g., exemplary modifications to the guide RNA.

### 1. Genetic Modifications to HLA-A

In some embodiments, the methods and compositions disclosed herein genetically modify at least one nucleotide of the HLA-A gene in a cell. In some embodiments, the genetic modification to HLA-A reduces or eliminates the expression of HLA-A protein on the surface of the genetically modified cell (or engineered cell). Genetic modifications encompass the population of modifications that results from contact with a genomic editing system (e.g., the population of edits that result from Cas9 and an HLA-A guide RNA, or the population of edits that result from BC22 and an HLA-A guide RNA).

In some embodiments, the genetic modification comprises at least one nucleotide within the genomic coordinates chr6:29942540-29945459. In some embodiments, the genetic modification comprises at least one nucleotide within the genomic coordinates chosen from any of the genomic coordinates listed in Table 1.

In some embodiments, the genetic modification comprises at least one nucleotide within the genomic coordinates chosen from: chr6:29942891-29942915; chr6:29942609-29942633; chr6:29944266-29944290; chr6:29942889-29942913; chr6:29944471-29944495; and chr6:29944470-29944494.

In some embodiments, the genetic modification comprises at least one nucleotide within the genomic coordinates chosen from: chr6:29942891-29942915; chr6:29942609-29942633; chr6:29942889-29942913; chr6:29944471-29944495; chr6:29944266-29944290; chr6:29942785-29942809

In some embodiments, the genetic modification comprises at least 5 contiguous nucleotides within the genomic coordinates chosen from: chr6:29942891-29942915; chr6:29942609-29942633; chr6:29944266-29944290; chr6:29942889-29942913; chr6:29944471-29944495; and chr6:29944470-29944494. In some embodiments, the genetic modification comprises at least 10 contiguous nucleotides within the genomic coordinates chosen from: chr6:29942891-29942915; chr6:29942609-29942633; chr6:29944266-29944290; chr6:29942889-29942913; chr6:29944471-29944495; chr6:29944470-29944494. In some embodiments, the genetic modification comprises at least one indel, at least one C to T substitution, or at least one A to G substitution within the genomic coordinates chosen from: chr6:29942891-29942915; chr6:29942609-29942633; chr6:29944266-29944290; chr6:29942889-29942913; chr6:29944471-29944495; chr6:29944470-29944494.

In some embodiments, the genetic modification comprises at least 5 contiguous nucleotides within the genomic coordinates chosen from: chr6:29942891-29942915; chr6:29942609-29942633; chr6:29942889-29942913; chr6:29944471-29944495; chr6:29944266-29944290; chr6:29942785-29942809 In some embodiments, the genetic modification comprises at least 10 contiguous nucleotides within the genomic coordinates chosen from: chr6:29942891-29942915; chr6:29942609-29942633; chr6:29942889-29942913; chr6:29944471-29944495; chr6:29944266-29944290; chr6:29942785-29942809 In some embodiments, the genetic modification comprises at least one indel, at least one C to T substitution, or at least one A to G substitution within the genomic coordinates chosen from: chr6:29942891-29942915; chr6:29942609-29942633; chr6:29942889-29942913; chr6:29944471-29944495; chr6:29944266-29944290; chr6:29942785-29942809

In some embodiments, the modification to HLA-A comprises any one or more of an insertion, deletion, substitution, or deamination of at least one nucleotide in a target sequence. In some embodiments, the modification to HLA-A comprises an insertion of 1, 2, 3, 4 or 5 or more nucleotides in a target sequence. In some embodiments, the modification to HLA-A comprises a deletion of 1, 2, 3, 4 or 5 or more nucleotides in a target sequence. In other embodiments, the modification to HLA-A comprises an insertion of 1, 2, 3, 4, 5, 6, 7, 8, 9, 10, 15, 20 or 25 or more nucleotides in a target sequence. In other embodiments, the modification to HLA-A comprises a deletion of 1, 2, 3, 4, 5, 6, 7, 8, 9, 10, 15, 20 or 25 or more nucleotides in a target sequence. In some embodiments, the modification to HLA-A comprises an indel, which is generally defined in the art as an insertion or deletion of less than 1000 base pairs (bp). In some embodiments, the modification to HLA-A comprises an indel which results in a frameshift mutation in a target sequence. In some embodiments, the modification to HLA-A comprises a substitution of 1, 2, 3, 4, 5, 6, 7, 8, 9, 10, 15, 20 or 25 or more nucleotides in a target sequence. In some embodiments, the modification to HLA-A comprises one or more of an insertion, deletion, or substitution of nucleotides resulting from the incorporation of a template nucleic acid. In some embodiments, the modification to HLA-A is not transient.

In some embodiments, the methods and compositions disclosed herein modify the HLA-A gene in a cell using an RNA-guided DNA binding agent (e.g., a Cas enzyme). In some embodiments, the RNA-guided DNA binding agent is Cas9. In some embodiments, the RNA-guided DNA binding agent cuts within the HLA-A gene, wherein the HLA-A guide RNA targets an HLA-A genomic target sequence comprising at least 10 contiguous nucleotides within the genomic coordinates chr6:29942540-29945459.

In some embodiments, the genetic modification to HLA-A results in utilization of an out-of-frame stop codon. In some embodiments, the genetic modification to HLA-A results in exon skipping during splicing. In some embodiments, the genetic modification to HLA-A results in reduced HLA-A protein expression by the cell. In some embodiments, the modification to the HLA-A results in reduced or eliminated HLA-A protein expression on the surface of the cell.

In some embodiments, HLA-A expression on the surface of a cell is reduced as a result of the genetic modification to HLA-A. In some embodiments, HLA-A expression on the surface of a cell is absent as a result of the genetic modification to HLA-A.

### 2. Efficacy of HLA-A Guide RNAs

The efficacy of an HLA-A guide RNA may be determined by techniques available in the art that assess the editing efficiency of a guide RNA, the levels of HLA-A mRNA, or the levels of HLA-A protein in a target cell. In some embodiments, the reduction or elimination of HLA-A protein on the surface of a cell may be determined by comparison to an unmodified cell (or “relative to an unmodified cell”). An engineered cell or cell population may also be compared to a population of unmodified cells.

An “unmodified cell” (or “unmodified cells”) refers to a control cell (or cells) of the same type of cell in an experiment or test, wherein the “unmodified” control cell has not been contacted with an HLA-A guide (i.e., a non-engineered cell). Therefore, an unmodified cell (or cells) may be a cell that has not been contacted with a guide RNA, or a cell that has been contacted with a guide RNA that does not target HLA-A.

In some embodiments, the efficacy of an HLA-A guide RNA is determined by measuring the reduction or elimination of HLA-A protein on the surface of the target cells). In some embodiments, HLA-A protein expression is measured by flow cytometry (e.g., with an antibody against HLA-A2/HLA-A3). In some embodiments, the population of cells is enriched (e.g., by FACS or MACS) and is at least 65%, 70%, 80%, 90%, 91%, 92%, 93%, or 94% HLA-A negative as measured by flow cytometry relative to a population of unmodified cells. In some embodiments, the population of cells is not enriched (e.g., by FACS or MACS) and is at least 65%, 70%, 80%, 90%, 91%, 92%, 93%, or 94% HLA-A negative as measured by flow cytometry relative to a population of unmodified cells.

In some embodiments, the population of cells is at least 65% HLA-A negative as measured by flow cytometry relative to a population of unmodified cells. In some embodiments, the population of cells is at least 70% HLA-A negative as measured by flow cytometry relative to a population of unmodified cells. In some embodiments, the population of cells is at least 80% HLA-A negative as measured by flow cytometry relative to a population of unmodified cells. In some embodiments, the population of cells is at least 90% HLA-A negative as measured by flow cytometry relative to a population of unmodified cells. In some embodiments, the population of cells is at least 91% HLA-A negative as measured by flow cytometry relative to a population of unmodified cells. In some embodiments, the population of cells is at least 92% HLA-A negative as measured by flow cytometry relative to a population of unmodified cells. In some embodiments, the population of cells is at least 93% HLA-A negative as measured by flow cytometry relative to a population of unmodified cells. In some embodiments, the population of cells is at least 94% HLA-A negative as measured by flow cytometry relative to a population of unmodified cells.

In some embodiments, an effective HLA-A guide RNA may be determined by measuring the response of immune cells in vitro or in vivo (e.g., CD8+ T cells) to the genetically modified target cell. For example, a reduced response from CD8+ T cells is indicative of an effective HLA-A guide RNA. A CD8+ T cell response may be evaluated by an assay that measures CD8+ T cell activation responses, e.g., CD8+ T cell proliferation, expression of activation markers, and/or cytokine production (IL-2, IFN-γ, TNF-α) (e.g., flow cytometry, ELISA). The CD8+ T cell response may be assessed in vitro or in vivo. In some embodiments, the CD8+ T cell response may be evaluated by co-culturing the genetically modified cell with CD8+ T cells in vitro. In some embodiments, CD8+ T cell activity may be evaluated in an in vivo model, e.g., a rodent model. In an in vivo model, e.g., genetically modified cells may be administered with CD8+ T cell; survival of the genetically modified cells is indicative of the ability to avoid CD8+ T cell lysis. In some embodiments, the methods produce a composition comprising a cell that survives in vivo in the presence of CD8+ T cells for greater than 1, 2, 3, 4, 5, or 6 weeks or more. In some embodiments, the methods produce a composition comprising a cell that survives in vivo in the presence of CD8+ T cells for at least one week to six weeks. In some embodiments, the methods produce a composition comprising a cell that survives in vivo in the presence of CD8+ T cells for at least two to four weeks. In some embodiments, the methods produce a composition comprising a cell that survives in vivo in the presence of CD8+ T cells for at least four to six weeks. In some embodiments, the methods produce a composition comprising a cell that survives in vivo in the presence of CD8+ T cells for more than six weeks.

The efficacy of an HLA-A guide RNA may also be assessed by the survival of the cell post-editing. In some embodiments, the cell survives post editing for at least one week to six weeks. In some embodiments, the cell survives post editing for at least two weeks. In some embodiments, the cell survives post editing for at least three weeks. In some embodiments, the cell survives post editing for at least four weeks. In some embodiments, the cell survives post editing for at least five weeks. In some embodiments, the cell survives post editing for at least six weeks. In some embodiments, the cell survives post editing for at least twelve weeks. The viability of a genetically modified cell may be measured using standard techniques, including e.g., by measures of cell death, by flow cytometry live/dead staining, or cell proliferation.

### D. TRAC Guide RNAs

The methods and compositions provided herein disclose TRAC guide RNAs useful for reducing the expression of TRAC protein on the surface of a cell. In some embodiments, such guide RNAs direct an RNA-guided DNA binding agent to a TRAC genomic target sequence and may be referred to herein as “TRAC guide RNAs.” In some embodiments, the TRAC guide RNA directs an RNA-guided DNA binding agent to a human TRAC genomic target sequence. In some embodiments, the TRAC guide RNA comprises a guide sequence selected from SEQ ID NO: 101-120.

In some embodiments, the methods and compositions disclosed herein comprise a TRAC guide RNA comprising a guide sequence that targets a TRAC genomic target sequence comprising at least 10 nucleotides within the genomic coordinates chr14:22547505-22551621 or chr14:22547462-22551621. In some embodiments, the method and composition disclosed herein comprise a TRAC guide RNA comprising a guide sequence that targets a TRAC genomic target sequence comprising at least one nucleotide within the genomic coordinates chr14:22547505-22551621 or chr14:22547462-22551621.

In some embodiments, the methods and compositions disclosed herein comprise a TRAC guide RNA comprising a guide sequence that directs an RNA-guided DNA binding agent to induce a double stranded break (DSB) or a single-stranded break (SSB) in a TRAC gene, wherein the TRAC guide RNA targets and TRAC genomic target sequence comprising at least 10 contiguous nucleotides within the genomic coordinates chr14:22547505-22551621 or chr14:22547462-22551621. In some embodiments, the methods and compositions disclosed herein comprise a TRAC guide RNA comprising a guide sequence that directs an RNA-guided DNA binding agent to induce a double stranded break (DSB) or a single-stranded break (SSB) in a TRAC gene, wherein the TRAC guide RNA targets a TRAC genomic target sequence comprising at least one nucleotide within the genomic coordinates chr14:22547505-22551621 or chr14:22547462-22551621.

In some embodiments, the methods and compositions disclose a TRAC guide RNA that directs an RNA-guided DNA binding agent to induce a double stranded break (DSB) or a single-stranded break (SSB) in a TRAC genomic target sequence. In some embodiments, the methods and compositions disclose a TRAC guide RNA that directs an RNA-guided DNA binding agent to make a cut in a TRAC genomic target sequence. In embodiments wherein the RNA-guided DNA cutting agent is Cas9, the cut or “cut site” occurs at the third base from the protospacer adjacent motif (PAM) sequence.

In some embodiments, a composition is provided comprising a TRAC guide RNA described herein and an RNA-guided DNA binding agent or a nucleic acid encoding an RNA-guided DNA binding agent.

In some embodiments, a composition is provided comprising a TRAC single-guide RNA (sgRNA) comprising a guide sequence that targets a genomic target comprising at least 10 contiguous nucleotides within the genomic coordinates chr14:22547505-22551621 or chr14:22547462-22551621. In some embodiments, a composition is provided comprising a TRAC sgRNA described herein and an RNA-guided DNA binding agent or a nucleic acid encoding an RNA-guided DNA binding agent.

In some embodiments, a composition is provided comprising a TRAC dual-guide RNA (dgRNA) comprising a guide sequence that targets a genomic target comprising at least 10 contiguous nucleotides within the genomic coordinates chr14:22547505-22551621 or chr14:22547462-22551621. In some embodiments, a composition is provided comprising a TRAC dgRNA described herein and an RNA-guided DNA binding agent or a nucleic acid encoding an RNA-guided DNA binding agent.

In some embodiments, the TRAC gRNA comprises a guide sequence selected from any one of SEQ ID NOs: 101-120. Exemplary TRAC guide sequences are shown below in Table 2 (SEQ ID NOs: 101-120) with corresponding guide RNA sequences 101-120.

TABLE 2
Exemplary TRAC guide sequences.

| | | | Exemplary | | |
|---|---|---|---|---|---|
| | SEQ ID | | Guide RNA | Exemplary Guide | |
| | NO to | | Full Sequence | RNA Modified | Genomic |
| Guide | the Guide | Guide | (SEQ ID Nos: | Sequence (SEQ ID | Coordinates |
| ID | Sequence | Sequence | 1101-1120) | NOs: 2101-2120) | (hg38) |
| G021469 | 101 | AUAUCCAGAAC | AUAUCCAGAAC | mA*mU*mA*mUmCCAm | chr14: 22547505- |
| | | CCUGACCCUGC | CCUGACCCUGC | GmAAmCCmCUGACmCC | 22547529 |
| | | CG | CGGUUGUAGCU | UGmCCGmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G021481 | 102 | GCCGUGUACCA | GCCGUGUACCA | mG*mC*mC*mGmUGUm | chr14: 22547525- |
| | | GCUGAGAGACU | GCUGAGAGACU | AmCCmAGmCUGAGmAG | 22547549 |
| | | CU | CUGUUGUAGCU | ACmUCUmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G028935 | 103 | CAGGCCACAGC | CAGGCCACAGC | mC*mA*mG*mGmCCAm | chr14: 22547674- |
| | | ACUGUUGCUCU | ACUGUUGCUCU | CmAGmCAmCUGUUmGC | 22547698 |
| | | UG | UGGUUGUAGCU | UCmUUGmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G028936 | 104 | CCCAGCCCAGG | CCCAGCCCAGG | mC*mC*mC*mAmGCCm | chr14: 22547768- |
| | | UAAGGGCAGCU | UAAGGGCAGCU | CmAGmGUmAAGGGmCA | 22547792 |
| | | UU | UUGUUGUAGCU | GCmUUUmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G028937 | 105 | GUCUUACCUGU | GUCUUACCUGU | mG*mU*mC*mUmUACm | chr14: 22549665- |
| | | UUCAAAGCUUU | UUCAAAGCUUU | CmUGmUUmUCAAAmGC | 22549689 |
| | | UC | UCGUUGUAGCU | UUmUUCmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G028938 | 106 | CUUUGAAACAG | CUUUGAAACAG | mC*mU*mU*mUmGAAm | chr14: 22549671- |
| | | GUAAGACAGGG | GUAAGACAGGG | AmCAmGGmUAAGAmCA | 22549695 |
| | | GU | GUGUUGUAGCU | GGmGGUmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G028939 | 107 | UUAGGUUCGUA | UUAGGUUCGUA | mU*mU*mA*mGmGUUm | chr14: 22550544- |
| | | UCUGUAAAACC | UCUGUAAAACC | CmGUmAUmCUGUAmAA | 22550568 |
| | | AA | AAGUUGUAGCU | ACmCAAmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G028940 | 108 | UUACAGAUACG | UUACAGAUACG | mU*mU*mA*mCmAGAm | chr14: 22550550- |
| | | AACCUAAACUU | AACCUAAACUU | UmACmGAmACCUAmAA | 22550574 |
| | | UC | UCGUUGUAGCU | CUmUUCmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G028941 | 109 | UUGAAAGUUUA | UUGAAAGUUUA | mU*mU*mG*mAmAAGm | chr14: 22550552- |
| | | GGUUCGUAUCU | GGUUCGUAUCU | UmUUmAGmGUUCGmUA | 22550576 |
| | | GU | GUGUUGUAGCU | UCmUGUmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G028942 | 110 | ACUUUCAAAAC | ACUUUCAAAAC | mA*mC*mU*mUmUCAm | chr14: 22550568- |
| | | CUGUCAGUGAU | CUGUCAGUGAU | AmAAmCCmUGUCAmGU | 22550592 |
| | | UG | UGGUUGUAGCU | GAmUUGmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G028943 | 111 | AAAACCUGUCA | AAAACCUGUCA | mA*mA*mA*mAmCCUm | chr14: 22550574- |
| | | GUGAUUGGGUU | GUGAUUGGGUU | GmUCmAGmUGAUUmGG | 22550598 |
| | | CC | CCGUUGUAGCU | GUmUCCmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G028944 | 112 | UGUCAGUGAUU | UGUCAGUGAUU | mU*mG*mU*mCmAGUm | chr14: 22550580- |
| | | GGGUUCCGAAU | GGGUUCCGAAU | GmAUmUGmGGUUCmCG | 22550604 |
| | | CC | CCGUUGUAGCU | AAmUCCmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G028945 | 113 | GGGUUCCGAAU | GGGUUCCGAAU | mG*mG*mG*mUmUCCm | chr14: 22550591- |
| | | CCUCCUCCUGA | CCUCCUCCUGA | GmAAmUCmCUCCUmCC | 22550615 |
| | | AA | AAGUUGUAGCU | UGmAAAmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G028946 | 114 | AAGGCCCCUCA | AAGGCCCCUCA | mA*mA*mG*mGmCCCm | chr14: 22550652- |
| | | CCUCAGCUGGA | CCUCAGCUGGA | CmUCmACmCUCAGmCU | 22550676 |
| | | CC | CCGUUGUAGCU | GGmACCmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G028947 | 115 | AGCUUCAAGGC | AGCUUCAAGGC | mA*mG*mC*mUmUCAm | chr14: 22550658- |
| | | CCCUCACCUCA | CCCUCACCUCA | AmGGmCCmCCUCAmCC | 22550682 |
| | | GC | GCGUUGUAGCU | UCmAGCmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G028948 | 116 | AUUCCAGAUCU | AUUCCAGAUCU | mA*mU*mU*mCmCAGm | chr14: 22551597- |
| | | GCAAGAUUGUA | GCAAGAUUGUA | AmUCmUGmCAAGAmUU | 22551621 |
| | | AG | AGGUUGUAGCU | GUmAAGmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G021475 | 117 | AACCCUGAUCC | AACCCUGAUCC | mA*mA*mC*mCmCUGm | chr14: 22547481- |
| | | UCUUGUCCCAC | UCUUGUCCCAC | AmUCmCUmCUUGUmCC | 22547505 |
| | | AG | AGGUUGUAGCU | CAmCAGmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G021476 | 118 | AUCAUGUCCUA | AUCAUGUCCUA | mA*mU*mC*mAmUGUm | chr14: 22547471- |
| | | ACCCUGAUCCU | ACCCUGAUCCU | CmCUmAAmCCCUGmAU | 22547495 |
| | | CU | CUGUUGUAGCU | CCmUCUmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G021477 | 119 | GAUCAUGUCCU | GAUCAUGUCCU | mG*mA*mU*mCmAUGm | chr14: 22547470- |
| | | AACCCUGAUCC | AACCCUGAUCC | UmCCmUAmACCCUmGA | 22547494 |
| | | UC | UCGUUGUAGCU | UCmCUCmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G021478 | 120 | GGAAAUGAGAU | GGAAAUGAGAU | mG*mG*mA*mAmAUGm | chr14: 22547462- |
| | | CAUGUCCUAAC | CAUGUCCUAAC | AmGAmUCmAUGUCmCU | 22547486 |
| | | CC | CCGUUGUAGCU | AAmCCCmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |

Throughout this application, the terms “mA,” “MC”, “mU,” or “mG” may be used to denote a nucleotide that has been modified with 2′—O-Me. Throughout this application, the terms A*, C*, U*, or G* may be used to denote a nucleotide that is linked to the next (e.g., 3′) nucleotide with a phosphorothioate (PS) bond.

In some embodiments, the TRAC guide RNA comprises a guide sequence selected from SEQ ID NOs: 101-120. In some embodiments, the TRAC guide RNA comprises a guide sequence that is at least 19, 20, 21, 22, 23, or 24 contiguous nucleotides of a sequence selected from SEQ ID NOs: 101-120. In some embodiments, the TRAC guide RNA comprises a guide sequence that is at least 95%, 90%, or 85% identical to a sequence selected from SEQ ID NOs: 101-120. In some embodiments, the TRAC guide RNA comprises a guide sequence that is at least 95% identical to a sequence selected from SEQ ID NOs: 101-120.

In some embodiments, the TRAC guide RNA comprises a guide sequence that comprises at least 10 contiguous nucleotides±10 nucleotides of a genomic coordinate listed in Table 2. As used herein, at least 10 contiguous nucleotides±10 nucleotides of a genomic coordinate means, for example, at least 10 contiguous nucleotides within the genomic coordinates wherein the genomic coordinates include 10 nucleotides in the 5′ direction and 10 nucleotides in the 3′ direction from the ranges listed in Table 2. For example, a TRAC guide RNA may comprise 10 contiguous nucleotides within the genomic coordinates chosen from: chr14:22547505-22547529; chr14:22547525-22547549; chr14:22547674-22547698; chr14:22550544-22550568; or chr14:22550574-22550598; including the boundary nucleotides of these ranges. As another example, a TRAC guide RNA may comprise 10 contiguous nucleotides within the genomic coordinates chosen from: chr14:22547481-22547505; chr14:22547471-22547495; chr14:22547470-22547494; or chr14:22547462-22547486; including the boundary nucleotides of these ranges. In some embodiments, the TRAC guide RNA comprises a guide sequence that is at least 19, 20, 21, 22, 23, or 24 contiguous nucleotides of a sequence that comprises 10 contiguous nucleotides±10 nucleotides of a genomic coordinate listed in Table 2. In some embodiments, the TRAC guide RNA comprises a guide sequence that is at least 95%, 90%, or 85% identical to a sequence selected from a sequence that is 19, 20, 21, 22, 23, or 24 contiguous nucleotides of a sequence that comprises 10 contiguous nucleotides±10 nucleotides of a genomic coordinate listed in Table 2.

In some embodiments, the TRAC guide RNA comprises a guide sequence that comprises at least 15 contiguous nucleotides±10 nucleotides of a genomic coordinate listed in Table 2. In some embodiments, the TRAC guide RNA comprises a guide sequence that comprises at least 24 contiguous nucleotides±10 nucleotides of a genomic coordinate listed in Table 2.

In some embodiments, the TRAC guide RNA comprises SEQ ID NO: 101. In some embodiments, the TRAC guide RNA comprises SEQ ID NO: 102. In some embodiments, the TRAC guide RNA comprises SEQ ID NO: 103. In some embodiments, the TRAC guide RNA comprises SEQ ID NO: 104. In some embodiments, the TRAC guide RNA comprises SEQ ID NO: 105. In some embodiments, the TRAC guide RNA comprises SEQ ID NO: 106. In some embodiments, the TRAC guide RNA comprises SEQ ID NO: 107. In some embodiments, the TRAC guide RNA comprises SEQ ID NO: 108. In some embodiments, the TRAC guide RNA comprises SEQ ID NO: 109. In some embodiments, the TRAC guide RNA comprises SEQ ID NO: 110. In some embodiments, the TRAC guide RNA comprises SEQ ID NO: 111. In some embodiments, the TRAC guide RNA comprises SEQ ID NO: 112. In some embodiments, the TRAC guide RNA comprises SEQ ID NO: 113. In some embodiments, the TRAC guide RNA comprises SEQ ID NO: 114. In some embodiments, the TRAC guide RNA comprises SEQ ID NO: 115. In some embodiments, the TRAC guide RNA comprises SEQ ID NO: 116. In some embodiments, the TRAC guide RNA comprises SEQ ID NO: 117. In some embodiments, the TRAC guide RNA comprises SEQ ID NO: 118. In some embodiments, the TRAC guide RNA comprises SEQ ID NO: 119. In some embodiments, the TRAC guide RNA comprises SEQ ID NO: 120.

In some embodiments, the TRAC guide RNA comprises a nucleotide chosen from: SEQ ID NOs: 101-103, 107, 111, 117, or 118.

In some embodiments, the TRAC guide RNA comprises a sequence listed in Table 2. In some embodiments, the TRAC guide RNA comprises a sequence of any one of SEQ ID NOs: 101-120. In some embodiments, the TRAC guide RNA comprises a sequence of any one of SEQ ID NOs: 107, 111, and 117-120. In some embodiments, the TRAC guide RNA comprises a sequence of SEQ ID NO: 107. In some embodiments, the TRAC guide RNA comprises a sequence of SEQ ID NO: 111. In some embodiments, the TRAC guide RNA comprises a sequence of SEQ ID NO: 117. In some embodiments, the TRAC guide RNA comprises a sequence of SEQ ID NO: 118. In some embodiments, the TRAC guide RNA comprises a sequence of SEQ ID NO: 119. In some embodiments, the TRAC guide RNA comprises a sequence of SEQ ID NO: 120. In some embodiments, the TRAC guide RNA comprises a guide sequence comprising a sequence of any one of SEQ ID NOs: 101-120. In some embodiments, the TRAC guide RNA comprises a guide sequence comprising a sequence of any one of SEQ ID NOs: 107, 111, and 117-120. In some embodiments, the TRAC guide RNA comprises a guide sequence comprising a sequence of SEQ ID NO: 107. In some embodiments, the TRAC guide RNA comprises a guide sequence comprising a sequence of SEQ ID NO: 111. In some embodiments, the TRAC guide RNA comprises a guide sequence comprising a sequence of SEQ ID NO: 117. In some embodiments, the TRAC guide RNA comprises a guide sequence comprising a sequence of SEQ ID NO: 118. In some embodiments, the TRAC guide RNA comprises a guide sequence comprising a sequence of SEQ ID NO: 119. In some embodiments, the TRAC guide RNA comprises a guide sequence comprising a sequence of SEQ ID NO: 120. In some embodiments, the TRAC guide RNA comprises a sequence of any one of SEQ ID NOs: 1101-1120. In some embodiments, the TRAC guide RNA comprises a sequence of any one of SEQ ID NOs: 1107, 1111, and 1117-1120. In some embodiments, the TRAC guide RNA comprises a sequence of SEQ ID NO: 1107. In some embodiments, the TRAC guide RNA comprises a sequence of SEQ ID NO: 1111. In some embodiments, the TRAC guide RNA comprises a sequence of SEQ ID NO: 1117. In some embodiments, the TRAC guide RNA comprises a sequence of SEQ ID NO: 1118. In some embodiments, the TRAC guide RNA comprises a sequence of SEQ ID NO: 1119. In some embodiments, the TRAC guide RNA comprises a sequence of SEQ ID NO: 1120. In some embodiments, the TRAC guide RNA comprises a sequence of any one of SEQ ID NOs: 2101-2120, 3003, and 3004. In some embodiments, the TRAC guide RNA comprises a sequence of any one of SEQ ID NOs: 2107, 2111, 2117-2120, 3003, and 3004. In some embodiments, the TRAC guide RNA comprises a sequence of SEQ ID NO: 2107. In some embodiments, the TRAC guide RNA comprises a sequence of SEQ ID NO: 2111. In some embodiments, the TRAC guide RNA comprises a sequence of SEQ ID NO: 2117. In some embodiments, the TRAC guide RNA comprises a sequence of SEQ ID NO: 2118. In some embodiments, the TRAC guide RNA comprises a sequence of SEQ ID NO: 2119. In some embodiments, the TRAC guide RNA comprises a sequence of SEQ ID NO: 2120. In some embodiments, the TRAC guide RNA comprises a sequence of SEQ ID NO: 3003. In some embodiments, the TRAC guide RNA comprises a sequence of SEQ ID NO: 3004.

In some embodiments, the TRAC guide RNA is a single guide RNA (sgRNA) comprising a sequence of any one of the sgRNA sequences listed in Table 2.

Additional embodiments of TRAC guide RNAs are provided herein, including e.g., exemplary modifications to the guide RNA. 1. Genetic modifications to TRAC

In some embodiments, the methods and compositions disclosed herein genetically modify at least one nucleotide of the TRAC gene in a cell. In some embodiments, the genetic modification to TRAC reduces or eliminates the expression of TRAC protein on the surface of the genetically modified cell (or engineered cell). Genetic modifications encompass the population of modifications that results from contact with a genomic editing system (e.g., the population of edits that result from Cas9 and a TRAC guide RNA, or the population of edits that result from BC22 and a TRAC guide RNA).

In some embodiments, the genetic modification comprises at least one nucleotide within the genomic coordinates chr14:22547505-22551621 or chr14:22547462-22551621. In some embodiments, the genetic modification comprises at least one nucleotide within the genomic coordinates chosen from any of the genomic coordinates listed in Table 2.

In some embodiments, the genetic modification comprises at least one nucleotide within the genomic coordinates chosen from: chr14:22547505-22547529; chr14:22547525-22547549; chr14:22547674-22547698; chr14:22550544-22550568; or chr14:22550574-22550598. In some embodiments, the genetic modification comprises at least 5 contiguous nucleotides within the genomic coordinates chosen from: chr14:22547505-22547529; chr14:22547525-22547549; chr14:22547674-22547698; chr14:22550544-22550568; or chr14:22550574-22550598. In some embodiments, the genetic modification comprises at least 10 contiguous nucleotides within the genomic coordinates chosen from: chr14:22547505-22547529; chr14:22547525-22547549; chr14:22547674-22547698; chr14:22550544-22550568; or chr14:22550574-22550598. In some embodiments, the genetic modification comprises at least 10 contiguous nucleotides within the genomic coordinates chosen from: chr14:22547481-22547505; chr14:22547471-22547495; chr14:22547470-22547494; or chr14:22547462-22547486.

In some embodiments, the genetic modification comprises at least one indel, at least one C to T substitution, or at least one A to G substitution within the genomic coordinates chosen from: chr14:22547505-22547529; chr14:22547525-22547549; chr14:22547674-22547698; chr14:22550544-22550568; or chr14:22550574-22550598. In some embodiments, the genetic modification comprises at least one indel, at least one C to T substitution, or at least one A to G substitution within the genomic coordinates chosen from: chr14:22547481-22547505; chr14:22547471-22547495; chr14:22547470-22547494; or chr14:22547462-22547486.

In some embodiments, the modification to TRAC comprises any one or more of an insertion, deletion, substitution, or deamination of at least one nucleotide in a target sequence. In some embodiments, the modification to TRAC comprises an insertion of 1, 2, 3, 4 or 5 or more nucleotides in a target sequence. In some embodiments, the modification to TRAC comprises a deletion of 1, 2, 3, 4 or 5 or more nucleotides in a target sequence. In other embodiments, the modification to TRAC comprises an insertion of 1, 2, 3, 4, 5, 6, 7, 8, 9, 10, 15, 20 or 25 or more nucleotides in a target sequence. In other embodiments, the modification to TRAC comprises a deletion of 1, 2, 3, 4, 5, 6, 7, 8, 9, 10, 15, 20 or 25 or more nucleotides in a target sequence. In some embodiments, the modification to TRAC comprises an indel, which is generally defined in the art as an insertion or deletion of less than 1000 base pairs (bp). In some embodiments, the modification to TRAC comprises an indel which results in a frameshift mutation in a target sequence. In some embodiments, the modification to TRAC comprises a substitution of 1, 2, 3, 4, 5, 6, 7, 8, 9, 10, 15, 20 or 25 or more nucleotides in a target sequence. In some embodiments, the modification to TRAC comprises one or more of an insertion, deletion, or substitution of nucleotides resulting from the incorporation of a template nucleic acid. In some embodiments, the modification to TRAC comprises an insertion of a donor nucleic acid in a target sequence. In some embodiments, the modification to TRAC is not transient.

In some embodiments, the methods and compositions disclosed herein modify the TRAC gene in a cell using an RNA-guided DNA binding agent (e.g., a Cas enzyme). In some embodiments, the RNA-guided DNA binding agent is Cas9. In some embodiments, the RNA-guided DNA binding agent cuts within the TRAC gene, wherein the TRAC guide RNA targets a TRAC genomic target sequence comprising at least 10 contiguous nucleotides chr14:22547505-22551621 or chr14:22547462-22551621.

In some embodiments, the genetic modification to TRAC results in utilization of an out-of-frame stop codon. In some embodiments, the genetic modification to TRAC results in exon skipping during splicing. In some embodiments, the genetic modification to TRAC results in reduced TRAC protein expression by the cell. In some embodiments, the modification to the TRAC gene results in reduced or eliminated TRAC protein expression on the surface of the cell.

In some embodiments, TRAC expression on the surface of a cell is reduced as a result of the genetic modification to TRAC. In some embodiments, TRAC expression on the surface of a cell is absent as a result of the genetic modification to TRAC.

### 2. Efficacy of TRAC Guide RNAs

In some embodiments, the efficacy of a TRAC gRNA is determined when delivered or expressed together with other components forming an RNP. In some embodiments, the TRAC gRNA is expressed together with an RNA-guided DNA binding agent, such as a Cas protein, e.g. Cas9. In some embodiments, the TRAC gRNA is delivered to or expressed in a cell line that already stably expresses an RNA-guided DNA nuclease, such as a Cas nuclease or nickase, e.g. Cas9 nuclease or nickase. In some embodiments the TRAC gRNA is delivered to a cell as part of a RNP. In some embodiments, the TRAC gRNA is delivered to a cell along with a mRNA encoding an RNA-guided DNA nuclease, such as a Cas nuclease or nickase, e.g. Cas9 nuclease or nickase.

As described herein, use of an RNA-guided DNA nuclease and a TRAC guide RNA disclosed herein can lead to double-stranded breaks in the DNA which can produce errors in the form of insertion/deletion (indel) mutations upon repair by cellular machinery. Many mutations due to indels alter the reading frame or introduce premature stop codons and, therefore, produce a non-functional protein.

In some embodiments, the efficacy of particular TRAC gRNAs is determined based on in vitro models. In some embodiments, the in vitro model is HEK293 cells stably expressing Cas9 (HEK293_Cas9). In some embodiments the in vitro model is a peripheral blood mononuclear cell (PBMC). In some embodiments, the in vitro model is a T cell, such as primary human T cells. With respect to using primary cells, commercially available primary cells can be used to provide greater consistency between experiments. In some embodiments, the number of off-target sites at which a deletion or insertion occurs in an in vitro model (e.g., in T cell) is determined, e.g., by analyzing genomic DNA from transfected cells in vitro with Cas9 mRNA and the guide RNA. In some embodiments, such a determination comprises analyzing genomic DNA from the cells transfected in vitro with Cas9 mRNA, the TRAC guide RNA, and a donor oligonucleotide. Exemplary procedures for such determinations are provided in the working examples in which HEK293 cells, PBMCs, and human CD3+ T cells are used.

In some embodiments, the efficacy of particular TRAC gRNAs is determined across multiple in vitro cell models for a gRNA selection process. In some embodiments, a cell line comparison of data with selected TRAC gRNAs is performed. In some embodiments, cross screening in multiple cell models is performed.

In some embodiments, the efficacy of a TRAC guide RNA is measured by percent indels of TRAC. In some embodiments, the percent editing of TRAC is compared to the percent indels necessary to achieve knockdown of the TRAC protein products

In some embodiments, the efficacy of a guide RNA is measured by reduced or eliminated expression of a component of the T-cell receptor (TCR). In embodiments, the reduced or eliminated expression of a component of the T-cell receptor (TCR) includes reduced or eliminated expression of TRAC. In some embodiments, said reduced or eliminated expression of said component of the TCR is the result of introduction of one or more, e.g., one or two, e.g., one TRAC gRNA molecule described herein to said component of the TCR into said cell. In embodiments, said reduced or eliminated expression of a component of the TCR is as measured by flow cytometry, e.g., as described herein.

In some embodiments, the efficacy of a TRAC guide RNA is measured by the number and/or frequency of indels at off-target sequences within the genome of the target cell type, such as a T cell. In some embodiments, efficacious guide RNAs are provided which produce indels at off target sites at very low frequencies (e.g., <5%) in a cell population and/or relative to the frequency of indel creation at the target site. Thus, the disclosure provides for guide RNAs which do not exhibit off-target indel formation in the target cell type (e.g., a T cell), or which produce a frequency of off-target indel formation of <5% in a cell population and/or relative to the frequency of indel creation at the target site. In some embodiments, the disclosure provides guide RNAs which do not exhibit any off target indel formation in the target cell type (e.g., T cell). In some embodiments, guide RNAs are provided which produce indels at less than 5 off-target sites, e.g., as evaluated by one or more methods described herein. In some embodiments, guide RNAs are provided which produce indels at less than or equal to 4, 3, 2, or 1 off-target site(s) e.g., as evaluated by one or more methods described herein. In some embodiments, the off-target site(s) does not occur in a protein coding region in the target cell (e.g., hepatocyte) genome.

In some embodiments, detecting gene editing events, such as the formation of insertion/deletion (“indel”) mutations and homology directed repair (HDR) events in target DNA utilize linear amplification with a tagged primer and isolating the tagged amplification products (herein after referred to as “LAM-PCR,” or “Linear Amplification (LA)” method).

In some embodiments, the efficacy of a guide RNA is measured by the levels of functional protein complexes comprising the expressed protein product of the gene. In some embodiments, the efficacy of a guide RNA is measured by flow cytometric analysis of TCR expression by which the live population of edited cells is analyzed for loss of the TCR.

### E. TRBC Guide RNAs

The methods and compositions provided herein disclose TRBC guide RNAs useful for reducing the expression of TRBC1 protein or TRBC2 protein, or both, on the surface of a cell. In some embodiments, such guide RNAs direct an RNA-guided DNA binding agent to a TRBC1 genomic target sequence and may be referred herein as “TRBC1 guide RNAs.” In some embodiments, the TRBC1 guide RNA directs an RNA-guided DNA binding agent to a human TRBC1 genomic target sequence. In some embodiments, such guide RNAs direct an RNA-guided DNA binding agent to a TRBC2 genomic target sequence and may be referred herein as “TRBC2 guide RNAs.” In some embodiments, the TRBC2 guide RNA directs an RNA-guided DNA binding agent to a human TRBC2 genomic target sequence. In some embodiments, such guide RNAs direct an RNA-guided DNA binding agent to a TRBC1 genomic target sequence and to a TRBC2 genomic target sequence and may be referred herein as “TRBC1/2 guide RNAs.” In some embodiments, the TRBC1/2 guide RNA directs an RNA-guided DNA binding agent to a human TRBC1 genomic target sequence and to a human TRBC2 genomic target sequence. In some embodiments, a TRBC1 guide RNA may contain a mismatch to a TRBC2 genomic target sequence, but the TRBC1 guide RNA may still direct an RNA-guided DNA binding agent to a TRBC2 genomic target sequence. In some embodiments, a TRBC2 guide RNA may contain a mismatch to a TRBC1 genomic target sequence, but the TRBC2 guide RNA may still direct an RNA-guided DNA binding agent to a TRBC1 genomic target sequence.

In some embodiments, the methods and compositions disclosed herein comprise a TRBC1 guide RNA comprising a guide sequence that targets a TRBC1 genomic target sequence comprising at least 10 nucleotides within the genomic coordinates chr7:142791862-142793149. In some embodiments, the method and composition disclosed herein comprise a TRBC1 guide RNA comprising a guide sequence that targets a TRBC1 genomic target sequence comprising at least one nucleotide within the genomic coordinates chr7:142791862-142793149.

In some embodiments, the methods and compositions disclosed herein comprise a TRBC2 guide RNA comprising a guide sequence that targets a TRBC2 genomic target sequence comprising at least 10 nucleotides within the genomic coordinates chr7:142801104-142802543. In some embodiments, the method and composition disclosed herein comprise a TRBC2 guide RNA comprising a guide sequence that targets a TRBC2 genomic target sequence comprising at least one nucleotide within the genomic coordinates chr7:142801104-142802543.

In some embodiments, the methods and compositions disclosed herein comprise a TRBC1 guide RNA comprising a guide sequence that directs an RNA-guided DNA binding agent to induce a double stranded break (DSB) or a single-stranded break (SSB) in a TRBC1 gene, wherein the TRBC1 guide RNA targets and TRBC1 genomic target sequence comprising at least 10 contiguous nucleotides within the genomic coordinates chr7:142791862-142793149. In some embodiments, the methods and compositions disclosed herein comprise a TRBC1 guide RNA comprising a guide sequence that directs an RNA-guided DNA binding agent to induce a double stranded break (DSB) or a single-stranded break (SSB) in a TRBC1 gene, wherein the TRBC1 guide RNA targets a TRBC1 genomic target sequence comprising at least one nucleotide within the genomic coordinates chr7:142791862-142793149.

In some embodiments, the methods and compositions disclosed herein comprise a TRBC2 guide RNA comprising a guide sequence that directs an RNA-guided DNA binding agent to induce a double stranded break (DSB) or a single-stranded break (SSB) in a TRBC2 gene, wherein the TRBC2 guide RNA targets and TRBC2 genomic target sequence comprising at least 10 contiguous nucleotides within the genomic coordinates chr7:142801104-142802543. In some embodiments, the methods and compositions disclosed herein comprise a TRBC2 guide RNA comprising a guide sequence that directs an RNA-guided DNA binding agent to induce a double stranded break (DSB) or a single-stranded break (SSB) in a TRBC2 gene, wherein the TRBC2 guide RNA targets a TRBC2 genomic target sequence comprising at least one nucleotide within the genomic coordinates chr7:142801104-142802543.

In some embodiments, the methods and compositions disclose a TRBC1 guide RNA that directs an RNA-guided DNA binding agent to induce a double stranded break (DSB) or a single-stranded break (SSB) in a TRBC1 or TRBC2 genomic target sequence. In some embodiments, the methods and compositions disclose a TRBC1 or TRBC2 guide RNA that directs an RNA-guided DNA binding agent to make a cut in a TRBC1 or TRBC2 genomic target sequence. In embodiments wherein the RNA-guided DNA cutting agent is Cas9, the cut or “cut site” occurs at the third base from the protospacer adjacent motif (PAM) sequence.

In some embodiments, a composition is provided comprising a TRBC guide RNA described herein and an RNA-guided DNA binding agent or a nucleic acid encoding an RNA-guided DNA binding agent.

In some embodiments, a composition is provided comprising a TRBC1 single-guide RNA (sgRNA) comprising a guide sequence that targets a genomic target comprising at least 10 contiguous nucleotides within the genomic coordinates chr7:142791862-142793149. In some embodiments, a composition is provided comprising a TRBC1 sgRNA described herein and an RNA-guided DNA binding agent or a nucleic acid encoding an RNA-guided DNA binding agent.

In some embodiments, a composition is provided comprising a TRBC2 single-guide RNA (sgRNA) comprising a guide sequence that targets a genomic target comprising at least 10 contiguous nucleotides within the genomic coordinates chr7:142801104-142802543. In some embodiments, a composition is provided comprising a TRBC2 sgRNA described herein and an RNA-guided DNA binding agent or a nucleic acid encoding an RNA-guided DNA binding agent.

In some embodiments, a composition is provided comprising a TRBC1 dual-guide RNA (dgRNA) comprising a guide sequence that targets a genomic target comprising at least 10 contiguous nucleotides within the genomic coordinates chr7:142791862-142793149. In some embodiments, a composition is provided comprising a TRBC1 dgRNA described herein and an RNA-guided DNA binding agent or a nucleic acid encoding an RNA-guided DNA binding agent.

In some embodiments, a composition is provided comprising a TRBC2 dual-guide RNA (dgRNA) comprising a guide sequence that targets a genomic target comprising at least 10 contiguous nucleotides within the genomic coordinates chr7:142801104-142802543. In some embodiments, a composition is provided comprising a TRBC2 dgRNA described herein and an RNA-guided DNA binding agent or a nucleic acid encoding an RNA-guided DNA binding agent.

In some embodiments, the TRBC gRNA comprises a guide sequence selected from any one of SEQ ID NOs: 201-265. Exemplary TRBC guide sequences are shown below in Table 3 (SEQ ID NOs: 201-265) with corresponding guide RNA sequences 201-265.

TABLE 3
Exemplary TRBC1 and TRBC2 guide sequences.

| | | | | | Exemplary | |
|---|---|---|---|---|---|---|
| | | | | | Guide RNA | |
| | SEQ ID | | | | Modified | |
| | NO to | | | Exemplary Guide RNA | Sequence | Genomic |
| Guide | the Guide | | Guide | Full Sequence (SEQ | (SEQ ID NOs: | Coordinates |
| ID | Sequence | Target | Sequence | ID NOs: 1201-1265) | 2201-2265) | (hg38) |
| G028952 | 201 | TRBC1 | CACGGAC | CACGGACCCGCAGCCCCUC | mC*mA*mC* | chr7: |
| | | | CCGCAGC | AAGGAGUUGUAGCUCCCUG | mGmGACmCm | 142791862- |
| | | | CCCUCAA | AAACCGUUGCUACAAUAAG | CGmCAmGCC | 142791886 |
| | | | GGA | GCCGUCGAAAGAUGUGCCG | CCmUCAAmG | |
| | | | | CAACGCUCUGCCUUCUGGC | GAmGUUGmU | |
| | | | | AUCGUU | mAmGmCUCC | |
| | | | | | CmUmGmAmA | |
| | | | | | mAmCmCGUU | |
| | | | | | mGmCUAmCA | |
| | | | | | AU*AAGmGm | |
| | | | | | CCmGmUmCm | |
| | | | | | GmAmAmAmG | |
| | | | | | mAmUGUGCm | |
| | | | | | CGmCAAmCG | |
| | | | | | CUCUmGmCC | |
| | | | | | mUmUmCmUG | |
| | | | | | GCAUCG*mU | |
| | | | | | *mU | |
| G028953 | 202 | TRBC1 | ACGGACC | ACGGACCCGCAGCCCCUCA | mA*mC*mG* | chr7: |
| | | | CGCAGCC | AGGAGGUUGUAGCUCCCUG | mGmACCmCm | 142791863- |
| | | | CCUCAAG | AAACCGUUGCUACAAUAAG | GCmAGmCCC | 142791887 |
| | | | GAG | GCCGUCGAAAGAUGUGCCG | CUmCAAGmG | |
| | | | | CAACGCUCUGCCUUCUGGC | AGmGUUGmU | |
| | | | | AUCGUU | mAmGmCUCC | |
| | | | | | CmUmGmAmA | |
| | | | | | mAmCmCGUU | |
| | | | | | mGmCUAmCA | |
| | | | | | AU*AAGmGm | |
| | | | | | CCmGmUmCm | |
| | | | | | GmAmAmAmG | |
| | | | | | mAmUGUGCm | |
| | | | | | CGmCAAmCG | |
| | | | | | CUCUmGmCC | |
| | | | | | mUmUmCmUG | |
| | | | | | GCAUCG*mU | |
| | | | | | *mU | |
| G028968 | 203 | TRBC1 | GACGAGU | GACGAGUGGACCCAGGAUA | mG*mA*mC* | chr7: |
| | | | GGACCCA | GGGCCGUUGUAGCUCCCUG | mGmAGUmGm | 142792010- |
| | | | GGAUAGG | AAACCGUUGCUACAAUAAG | GAmCCmCAG | 142792034 |
| | | | GCC | GCCGUCGAAAGAUGUGCCG | GAmUAGGmG | |
| | | | | CAACGCUCUGCCUUCUGGC | CCmGUUGmU | |
| | | | | AUCGUU | mAmGmCUCC | |
| | | | | | CmUmGmAmA | |
| | | | | | mAmCmCGUU | |
| | | | | | mGmCUAmCA | |
| | | | | | AU*AAGmGm | |
| | | | | | CCmGmUmCm | |
| | | | | | GmAmAmAmG | |
| | | | | | mAmUGUGCm | |
| | | | | | CGmCAAmCG | |
| | | | | | CUCUmGmCC | |
| | | | | | mUmUmCmUG | |
| | | | | | GCAUCG*mU | |
| | | | | | *mU | |
| G028969 | 204 | TRBC1 | UGGACCC | UGGACCCAGGAUAGGGCCA | mU*mG*mG* | chr7: |
| | | | AGGAUAG | AACCCGUUGUAGCUCCCUG | mAmCCCmAm | 142792016- |
| | | | GGCCAAA | AAACCGUUGCUACAAUAAG | GGmAUmAGG | 142792040 |
| | | | CCC | GCCGUCGAAAGAUGUGCCG | GCmCAAAmC | |
| | | | | CAACGCUCUGCCUUCUGGC | CCmGUUGmU | |
| | | | | AUCGUU | mAmGmCUCC | |
| | | | | | CmUmGmAmA | |
| | | | | | mAmCmCGUU | |
| | | | | | mGmCUAmCA | |
| | | | | | AU*AAGmGm | |
| | | | | | CCmGmUmCm | |
| | | | | | GmAmAmAmG | |
| | | | | | mAmUGUGCm | |
| | | | | | CGmCAAmCG | |
| | | | | | CUCUmGmCC | |
| | | | | | mUmUmCmUG | |
| | | | | | GCAUCG*mU | |
| | | | | | *mU | |
| G028970 | 205 | TRBC1 | GGACCCA | GGACCCAGGAUAGGGCCAA | mG*mG*mA* | chr7: |
| | | | GGAUAGG | ACCCGGUUGUAGCUCCCUG | mCmCCAmGm | 142792017- |
| | | | GCCAAAC | AAACCGUUGCUACAAUAAG | GAmUAmGGG | 142792041 |
| | | | CCG | GCCGUCGAAAGAUGUGCCG | CCmAAACmC | |
| | | | | CAACGCUCUGCCUUCUGGC | CGmGUUGmU | |
| | | | | AUCGUU | mAmGmCUCC | |
| | | | | | CmUmGmAmA | |
| | | | | | mAmCmCGUU | |
| | | | | | mGmCUAmCA | |
| | | | | | AU*AAGmGm | |
| | | | | | CCmGmUmCm | |
| | | | | | GmAmAmAmG | |
| | | | | | mAmUGUGCm | |
| | | | | | CGmCAAmCG | |
| | | | | | CUCUmGmCC | |
| | | | | | mUmUmCmUG | |
| | | | | | GCAUCG*mU | |
| | | | | | *mU | |
| G028977 | 206 | TRBC1 | CCCUGCU | CCCUGCUUUCUUUCAGACU | mC*mC*mC* | chr7: |
| | | | UUCUUUC | GUGGCGUUGUAGCUCCCUG | mUmGCUmUm | 142792505- |
| | | | AGACUGU | AAACCGUUGCUACAAUAAG | UCmUUmUCA | 142792529 |
| | | | GGC | GCCGUCGAAAGAUGUGCCG | GAmCUGUmG | |
| | | | | CAACGCUCUGCCUUCUGGC | GCmGUUGmU | |
| | | | | AUCGUU | mAmGmCUCC | |
| | | | | | CmUmGmAmA | |
| | | | | | mAmCmCGUU | |
| | | | | | mGmCUAmCA | |
| | | | | | AU*AAGmGm | |
| | | | | | CCmGmUmCm | |
| | | | | | GmAmAmAmG | |
| | | | | | mAmUGUGCm | |
| | | | | | CGmCAAmCG | |
| | | | | | CUCUmGmCC | |
| | | | | | mUmUmCmUG | |
| | | | | | GCAUCG*mU | |
| | | | | | *mU | |
| G028978 | 207 | TRBC1 | GACUGUG | GACUGUGGCUUUACCUCGG | mG*mA*mC* | chr7: |
| | | | GCUUUAC | GUAAGGUUGUAGCUCCCUG | mUmGUGmGm | 142792520- |
| | | | CUCGGGU | AAACCGUUGCUACAAUAAG | CUmUUmACC | 142792544 |
| | | | AAG | GCCGUCGAAAGAUGUGCCG | UCmGGGUmA | |
| | | | | CAACGCUCUGCCUUCUGGC | AGmGUUGmU | |
| | | | | AUCGUU | mAmGmCUCC | |
| | | | | | CmUmGmAmA | |
| | | | | | mAmCmCGUU | |
| | | | | | mGmCUAmCA | |
| | | | | | AU*AAGmGm | |
| | | | | | CCmGmUmCm | |
| | | | | | GmAmAmAmG | |
| | | | | | mAmUGUGCm | |
| | | | | | CGmCAAmCG | |
| | | | | | CUCUmGmCC | |
| | | | | | mUmUmCmUG | |
| | | | | | GCAUCG*mU | |
| | | | | | *mU | |
| G028979 | 208 | TRBC1 | ACUGUGG | ACUGUGGCUUUACCUCGGG | mA*mC*mU* | chr7: |
| | | | CUUUACC | UAAGUGUUGUAGCUCCCUG | mGmUGGmCm | 142792521- |
| | | | UCGGGUA | AAACCGUUGCUACAAUAAG | UUmUAmCCU | 142792545 |
| | | | AGU | GCCGUCGAAAGAUGUGCCG | CGmGGUAmA | |
| | | | | CAACGCUCUGCCUUCUGGC | GUmGUUGmU | |
| | | | | AUCGUU | mAmGmCUCC | |
| | | | | | CmUmGmAmA | |
| | | | | | mAmCmCGUU | |
| | | | | | mGmCUAmCA | |
| | | | | | AU*AAGmGm | |
| | | | | | CCmGmUmCm | |
| | | | | | GmAmAmAmG | |
| | | | | | mAmUGUGCm | |
| | | | | | CGmCAAmCG | |
| | | | | | CUCUmGmCC | |
| | | | | | mUmUmCmUG | |
| | | | | | GCAUCG*mU | |
| | | | | | *mU | |
| G028980 | 209 | TRBC1 | UGGCUUU | UGGCUUUACCUCGGGUAAG | mU*mG*mG* | chr7: |
| | | | ACCUCGG | UAAGCGUUGUAGCUCCCUG | mCmUUUmAm | 142792525- |
| | | | GUAAGUA | AAACCGUUGCUACAAUAAG | CCmUCmGGG | 142792549 |
| | | | AGC | GCCGUCGAAAGAUGUGCCG | UAmAGUAmA | |
| | | | | CAACGCUCUGCCUUCUGGC | GCmGUUGmU | |
| | | | | AUCGUU | mAmGmCUCC | |
| | | | | | CmUmGmAmA | |
| | | | | | mAmCmCGUU | |
| | | | | | mGmCUAmCA | |
| | | | | | AU*AAGmGm | |
| | | | | | CCmGmUmCm | |
| | | | | | GmAmAmAmG | |
| | | | | | mAmUGUGCm | |
| | | | | | CGmCAAmCG | |
| | | | | | CUCUmGmCC | |
| | | | | | mUmUmCmUG | |
| | | | | | GCAUCG*mU | |
| | | | | | *mU | |
| G028981 | 210 | TRBC1 | UACCUCG | UACCUCGGGUAAGUAAGCC | mU*mA*mC* | chr7: |
| | | | GGUAAGU | CUUCCGUUGUAGCUCCCUG | mCmUCGmGm | 142792531- |
| | | | AAGCCCU | AAACCGUUGCUACAAUAAG | GUmAAmGUA | 142792555 |
| | | | UCC | GCCGUCGAAAGAUGUGCCG | AGmCCCUmU | |
| | | | | CAACGCUCUGCCUUCUGGC | CCmGUUGmU | |
| | | | | AUCGUU | mAmGmCUCC | |
| | | | | | CmUmGmAmA | |
| | | | | | mAmCmCGUU | |
| | | | | | mGmCUAmCA | |
| | | | | | AU*AAGmGm | |
| | | | | | CCmGmUmCm | |
| | | | | | GmAmAmAmG | |
| | | | | | mAmUGUGCm | |
| | | | | | CGmCAAmCG | |
| | | | | | CUCUmGmCC | |
| | | | | | mUmUmCmUG | |
| | | | | | GCAUCG*mU | |
| | | | | | *mU | |
| G028982 | 211 | TRBC1 | AGGAAGG | AGGAAGGGCUUACUUACCC | mA*mG*mG* | chr7: |
| | | | GCUUACU | GAGGUGUUGUAGCUCCCUG | mAmAGGmGm | 142792532- |
| | | | UACCCGA | AAACCGUUGCUACAAUAAG | CUmUAmCUU | 142792556 |
| | | | GGU | GCCGUCGAAAGAUGUGCCG | ACmCCGAmG | |
| | | | | CAACGCUCUGCCUUCUGGC | GUmGUUGmU | |
| | | | | AUCGUU | mAmGmCUCC | |
| | | | | | CmUmGmAmA | |
| | | | | | mAmCmCGUU | |
| | | | | | mGmCUAmCA | |
| | | | | | AU*AAGmGm | |
| | | | | | CCmGmUmCm | |
| | | | | | GmAmAmAmG | |
| | | | | | mAmUGUGCm | |
| | | | | | CGmCAAmCG | |
| | | | | | CUCUmGmCC | |
| | | | | | mUmUmCmUG | |
| | | | | | GCAUCG*mU | |
| | | | | | *mU | |
| G028983 | 212 | TRBC1 | CGGGUAA | CGGGUAAGUAAGCCCUUCC | mC*mG*mG* | chr7: |
| | | | GUAAGCC | UUUUCGUUGUAGCUCCCUG | mGmUAAmGm | 142792536- |
| | | | CUUCCUU | AAACCGUUGCUACAAUAAG | UAmAGmCCC | 142792560 |
| | | | UUC | GCCGUCGAAAGAUGUGCCG | UUmCCUUmU | |
| | | | | CAACGCUCUGCCUUCUGGC | UCmGUUGmU | |
| | | | | AUCGUU | mAmGmCUCC | |
| | | | | | CmUmGmAmA | |
| | | | | | mAmCmCGUU | |
| | | | | | mGmCUAmCA | |
| | | | | | AU*AAGmGm | |
| | | | | | CCmGmUmCm | |
| | | | | | GmAmAmAmG | |
| | | | | | mAmUGUGCm | |
| | | | | | CGmCAAmCG | |
| | | | | | CUCUmGmCC | |
| | | | | | mUmUmCmUG | |
| | | | | | GCAUCG*mU | |
| | | | | | *mU | |
| G028984 | 213 | TRBC1 | GGGUAAG | GGGUAAGUAAGCCCUUCCU | mG*mG*mG* | chr7: |
| | | | UAAGCCC | UUUCCGUUGUAGCUCCCUG | mUmAAGmUm | 142792537- |
| | | | UUCCUUU | AAACCGUUGCUACAAUAAG | AAmGCmCCU | 142792561 |
| | | | UCC | GCCGUCGAAAGAUGUGCCG | UCmCUUUmU | |
| | | | | CAACGCUCUGCCUUCUGGC | CCmGUUGmU | |
| | | | | AUCGUU | mAmGmCUCC | |
| | | | | | CmUmGmAmA | |
| | | | | | mAmCmCGUU | |
| | | | | | mGmCUAmCA | |
| | | | | | AU*AAGmGm | |
| | | | | | CCmGmUmCm | |
| | | | | | GmAmAmAmG | |
| | | | | | mAmUGUGCm | |
| | | | | | CGmCAAmCG | |
| | | | | | CUCUmGmCC | |
| | | | | | mUmUmCmUG | |
| | | | | | GCAUCG*mU | |
| | | | | | *mU | |
| G028985 | 214 | TRBC1 | UGCCAAC | UGCCAACAGUGUCCUACCA | mU*mG*mC* | chr7: |
| | | | AGUGUCC | GCAAGGUUGUAGCUCCCUG | mCmAACmAm | 142792682- |
| | | | UACCAGC | AAACCGUUGCUACAAUAAG | GUmGUmCCU | 142792706 |
| | | | AAG | GCCGUCGAAAGAUGUGCCG | ACmCAGCmA | |
| | | | | CAACGCUCUGCCUUCUGGC | AGmGUUGmU | |
| | | | | AUCGUU | mAmGmCUCC | |
| | | | | | CmUmGmAmA | |
| | | | | | mAmCmCGUU | |
| | | | | | mGmCUAmCA | |
| | | | | | AU*AAGmGm | |
| | | | | | CCmGmUmCm | |
| | | | | | GmAmAmAmG | |
| | | | | | mAmUGUGCm | |
| | | | | | CGmCAAmCG | |
| | | | | | CUCUmGmCC | |
| | | | | | mUmUmCmUG | |
| | | | | | GCAUCG*mU | |
| | | | | | *mU | |
| G028986 | 215 | TRBC1 | GUGUCCU | GUGUCCUACCAGCAAGGGG | mG*mU*mG* | chr7: |
| | | | ACCAGCA | UCCUGGUUGUAGCUCCCUG | mUmCCUmAm | 142792690- |
| | | | AGGGGUC | AAACCGUUGCUACAAUAAG | CCmAGmCAA | 142792714 |
| | | | CUG | GCCGUCGAAAGAUGUGCCG | GGmGGUCmC | |
| | | | | CAACGCUCUGCCUUCUGGC | UGmGUUGmU | |
| | | | | AUCGUU | mAmGmCUCC | |
| | | | | | CmUmGmAmA | |
| | | | | | mAmCmCGUU | |
| | | | | | mGmCUAmCA | |
| | | | | | AU*AAGmGm | |
| | | | | | CCmGmUmCm | |
| | | | | | GmAmAmAmG | |
| | | | | | mAmUGUGCm | |
| | | | | | CGmCAAmCG | |
| | | | | | CUCUmGmCC | |
| | | | | | mUmUmCmUG | |
| | | | | | GCAUCG*mU | |
| | | | | | *mU | |
| G028987 | 216 | TRBC1 | UCCUACC | UCCUACCAGCAAGGGGUCC | mU*mC*mC* | chr7: |
| | | | AGCAAGG | UGUCUGUUGUAGCUCCCUG | mUmACCmAm | 142792693- |
| | | | GGUCCUG | AAACCGUUGCUACAAUAAG | GCmAAmGGG | 142792717 |
| | | | UCU | GCCGUCGAAAGAUGUGCCG | GUmCCUGmU | |
| | | | | CAACGCUCUGCCUUCUGGC | CUmGUUGmU | |
| | | | | AUCGUU | mAmGmCUCC | |
| | | | | | CmUmGmAmA | |
| | | | | | mAmCmCGUU | |
| | | | | | mGmCUAmCA | |
| | | | | | AU*AAGmGm | |
| | | | | | CCmGmUmCm | |
| | | | | | GmAmAmAmG | |
| | | | | | mAmUGUGCm | |
| | | | | | CGmCAAmCG | |
| | | | | | CUCUmGmCC | |
| | | | | | mUmUmCmUG | |
| | | | | | GCAUCG*mU | |
| | | | | | *mU | |
| G028989 | 217 | TRBC1 | ACCAUGG | ACCAUGGCCAUCAACACAA | mA*mC*mC* | chr7: |
| | | | CCAUCAA | GGGCGGUUGUAGCUCCCUG | mAmUGGmCm | 142792776- |
| | | | CACAAGG | AAACCGUUGCUACAAUAAG | CAmUCmAAC | 142792800 |
| | | | GCG | GCCGUCGAAAGAUGUGCCG | ACmAAGGmG | |
| | | | | CAACGCUCUGCCUUCUGGC | CGmGUUGmU | |
| | | | | AUCGUU | mAmGmCUCC | |
| | | | | | CmUmGmAmA | |
| | | | | | mAmCmCGUU | |
| | | | | | mGmCUAmCA | |
| | | | | | AU*AAGmGm | |
| | | | | | CCmGmUmCm | |
| | | | | | GmAmAmAmG | |
| | | | | | mAmUGUGCm | |
| | | | | | CGmCAAmCG | |
| | | | | | CUCUmGmCC | |
| | | | | | mUmUmCmUG | |
| | | | | | GCAUCG*mU | |
| | | | | | *mU | |
| G028990 | 218 | TRBC1 | CCAUGGU | CCAUGGUAAGCAGGAGGGC | mC*mC*mA* | chr7: |
| | | | AAGCAGG | AGGAUGUUGUAGCUCCCUG | mUmGGUmAm | 142792793- |
| | | | AGGGCAG | AAACCGUUGCUACAAUAAG | AGmCAmGGA | 142792817 |
| | | | GAU | GCCGUCGAAAGAUGUGCCG | GGmGCAGmG | |
| | | | | CAACGCUCUGCCUUCUGGC | AUmGUUGmU | |
| | | | | AUCGUU | mAmGmCUCC | |
| | | | | | CmUmGmAmA | |
| | | | | | mAmCmCGUU | |
| | | | | | mGmCUAmCA | |
| | | | | | AU*AAGmGm | |
| | | | | | CCmGmUmCm | |
| | | | | | GmAmAmAmG | |
| | | | | | mAmUGUGCm | |
| | | | | | CGmCAAmCG | |
| | | | | | CUCUmGmCC | |
| | | | | | mUmUmCmUG | |
| | | | | | GCAUCG*mU | |
| | | | | | *mU | |
| G028991 | 219 | TRBC1 | GGUCAAG | GGUCAAGAGAAAGGAUUUC | mG*mG*mU* | chr7: |
| | | | AGAAAGG | UGAAGGUUGUAGCUCCCUG | mCmAAGmAm | 142793119- |
| | | | AUUUCUG | AAACCGUUGCUACAAUAAG | GAmAAmGGA | 142793143 |
| | | | AAG | GCCGUCGAAAGAUGUGCCG | UUmUCUGmA | |
| | | | | CAACGCUCUGCCUUCUGGC | AGmGUUGmU | |
| | | | | AUCGUU | mAmGmCUCC | |
| | | | | | CmUmGmAmA | |
| | | | | | mAmCmCGUU | |
| | | | | | mGmCUAmCA | |
| | | | | | AU*AAGmGm | |
| | | | | | CCmGmUmCm | |
| | | | | | GmAmAmAmG | |
| | | | | | mAmUGUGCm | |
| | | | | | CGmCAAmCG | |
| | | | | | CUCUmGmCC | |
| | | | | | mUmUmCmUG | |
| | | | | | GCAUCG*mU | |
| | | | | | *mU | |
| G028992 | 220 | TRBC1 | GUCAAGA | GUCAAGAGAAAGGAUUUCU | mG*mU*mC* | chr7: |
| | | | GAAAGGA | GAAGGGUUGUAGCUCCCUG | mAmAGAmGm | 142793120- |
| | | | UUUCUGA | AAACCGUUGCUACAAUAAG | AAmAGmGAU | 142793144 |
| | | | AGG | GCCGUCGAAAGAUGUGCCG | UUmCUGAmA | |
| | | | | CAACGCUCUGCCUUCUGGC | GGmGUUGmU | |
| | | | | AUCGUU | mAmGmCUCC | |
| | | | | | CmUmGmAmA | |
| | | | | | mAmCmCGUU | |
| | | | | | mGmCUAmCA | |
| | | | | | AU*AAGmGm | |
| | | | | | CCmGmUmCm | |
| | | | | | GmAmAmAmG | |
| | | | | | mAmUGUGCm | |
| | | | | | CGmCAAmCG | |
| | | | | | CUCUmGmCC | |
| | | | | | mUmUmCmUG | |
| | | | | | GCAUCG*mU | |
| | | | | | *mU | |
| G028993 | 221 | TRBC1 | GGCUGCC | GGCUGCCUUCAGAAAUCCU | mG*mG*mC* | chr7: |
| | | | UUCAGAA | UUCUCGUUGUAGCUCCCUG | mUmGCCmUm | 142793125- |
| | | | AUCCUUU | AAACCGUUGCUACAAUAAG | UCmAGmAAA | 142793149 |
| | | | CUC | GCCGUCGAAAGAUGUGCCG | UCmCUUUmC | |
| | | | | CAACGCUCUGCCUUCUGGC | UCmGUUGmU | |
| | | | | AUCGUU | mAmGmCUCC | |
| | | | | | CmUmGmAmA | |
| | | | | | mAmCmCGUU | |
| | | | | | mGmCUAmCA | |
| | | | | | AU*AAGmGm | |
| | | | | | CCmGmUmCm | |
| | | | | | GmAmAmAmG | |
| | | | | | mAmUGUGCm | |
| | | | | | CGmCAAmCG | |
| | | | | | CUCUmGmCC | |
| | | | | | mUmUmCmUG | |
| | | | | | GCAUCG*mU | |
| | | | | | *mU | |
| G028949 | 222 | TRBC1/2 | CCCACAC | CCCACACCCAAAAGGCCAC | mC*mC*mC* | chr7: |
| | | | CCAAAAG | ACUGGGUUGUAGCUCCCUG | mAmCACmCm | 142791756- |
| | | | GCCACAC | AAACCGUUGCUACAAUAAG | CAmAAmAGG | 142791780 |
| | | | UGG | GCCGUCGAAAGAUGUGCCG | CCmACACmU | |
| | | | | CAACGCUCUGCCUUCUGGC | GGmGUUGmU | |
| | | | | AUCGUU | mAmGmCUCC | |
| | | | | | CmUmGmAmA | |
| | | | | | mAmCmCGUU | |
| | | | | | mGmCUAmCA | |
| | | | | | AU*AAGmGm | |
| | | | | | CCmGmUmCm | |
| | | | | | GmAmAmAmG | |
| | | | | | mAmUGUGCm | |
| | | | | | CGmCAAmCG | |
| | | | | | CUCUmGmCC | |
| | | | | | mUmUmCmUG | |
| | | | | | GCAUCG*mU | |
| | | | | | *mU | |
| G028950 | 223 | TRBC1/2 | ACCCAAA | ACCCAAAAGGCCACACUGG | mA*mC*mC* | chr7: |
| | | | AGGCCAC | UGUGCGUUGUAGCUCCCUG | mCmAAAmAm | 142791761- |
| | | | ACUGGUG | AAACCGUUGCUACAAUAAG | GGmCCmACA | 142791785 |
| | | | UGC | GCCGUCGAAAGAUGUGCCG | CUmGGUGmU | |
| | | | | CAACGCUCUGCCUUCUGGC | GCmGUUGmU | |
| | | | | AUCGUU | mAmGmCUCC | |
| | | | | | CmUmGmAmA | |
| | | | | | mAmCmCGUU | |
| | | | | | mGmCUAmCA | |
| | | | | | AU*AAGmGm | |
| | | | | | CCmGmUmCm | |
| | | | | | GmAmAmAmG | |
| | | | | | mAmUGUGCm | |
| | | | | | CGmCAAmCG | |
| | | | | | CUCUmGmCC | |
| | | | | | mUmUmCmUG | |
| | | | | | GCAUCG*mU | |
| | | | | | *mU | |
| G028951 | 224 | TRBC1/2 | UCCUUCC | UCCUUCCCAUUCACCCACC | mU*mC*mC* | chr7: |
| | | | CAUUCAC | AGCUCGUUGUAGCUCCCUG | mUmUCCmCm | 142791820- |
| | | | CCACCAG | AAACCGUUGCUACAAUAAG | AUmUCmACC | 142791844 |
| | | | CUC | GCCGUCGAAAGAUGUGCCG | CAmCCAGmC | |
| | | | | CAACGCUCUGCCUUCUGGC | UCmGUUGmU | |
| | | | | AUCGUU | mAmGmCUCC | |
| | | | | | CmUmGmAmA | |
| | | | | | mAmCmCGUU | |
| | | | | | mGmCUAmCA | |
| | | | | | AU*AAGmGm | |
| | | | | | CCmGmUmCm | |
| | | | | | GmAmAmAmG | |
| | | | | | mAmUGUGCm | |
| | | | | | CGmCAAmCG | |
| | | | | | CUCUmGmCC | |
| | | | | | mUmUmCmUG | |
| | | | | | GCAUCG*mU | |
| | | | | | *mU | |
| G028954 | 225 | TRBC1/2 | GACCCGC | GACCCGCAGCCCCUCAAGG | mG*mA*mC* | chr7: |
| | | | AGCCCCU | AGCAGGUUGUAGCUCCCUG | mCmCGCmAm | 142791866- |
| | | | CAAGGAG | AAACCGUUGCUACAAUAAG | GCmCCmCUC | 142791890 |
| | | | CAG | GCCGUCGAAAGAUGUGCCG | AAmGGAGmC | |
| | | | | CAACGCUCUGCCUUCUGGC | AGmGUUGmU | |
| | | | | AUCGUU | mAmGmCUCC | |
| | | | | | CmUmGmAmA | |
| | | | | | mAmCmCGUU | |
| | | | | | mGmCUAmCA | |
| | | | | | AU*AAGmGm | |
| | | | | | CCmGmUmCm | |
| | | | | | GmAmAmAmG | |
| | | | | | mAmUGUGCm | |
| | | | | | CGmCAAmCG | |
| | | | | | CUCUmGmCC | |
| | | | | | mUmUmCmUG | |
| | | | | | GCAUCG*mU | |
| | | | | | *mU | |
| G028955 | 226 | TRBC1/2 | ACCCGCA | ACCCGCAGCCCCUCAAGGA | mA*mC*mC* | chr7: |
| | | | GCCCCUC | GCAGCGUUGUAGCUCCCUG | mCmGCAmGm | 142791867- |
| | | | AAGGAGC | AAACCGUUGCUACAAUAAG | CCmCCmUCA | 142791891 |
| | | | AGC | GCCGUCGAAAGAUGUGCCG | AGmGAGCmA | |
| | | | | CAACGCUCUGCCUUCUGGC | GCmGUUGmU | |
| | | | | AUCGUU | mAmGmCUCC | |
| | | | | | CmUmGmAmA | |
| | | | | | mAmCmCGUU | |
| | | | | | mGmCUAmCA | |
| | | | | | AU*AAGmGm | |
| | | | | | CCmGmUmCm | |
| | | | | | GmAmAmAmG | |
| | | | | | mAmUGUGCm | |
| | | | | | CGmCAAmCG | |
| | | | | | CUCUmGmCC | |
| | | | | | mUmUmCmUG | |
| | | | | | GCAUCG*mU | |
| | | | | | *mU | |
| G028956 | 227 | TRBC1/2 | CUCAAGG | CUCAAGGAGCAGCCCGCCC | mC*mU*mC* | chr7: |
| | | | AGCAGCC | UCAAUGUUGUAGCUCCCUG | mAmAGGmAm | 142791878- |
| | | | CGCCCUC | AAACCGUUGCUACAAUAAG | GCmAGmCCC | 142791902 |
| | | | AAU | GCCGUCGAAAGAUGUGCCG | GCmCCUCmA | |
| | | | | CAACGCUCUGCCUUCUGGC | AUmGUUGmU | |
| | | | | AUCGUU | mAmGmCUCC | |
| | | | | | CmUmGmAmA | |
| | | | | | mAmCmCGUU | |
| | | | | | mGmCUAmCA | |
| | | | | | AU*AAGmGm | |
| | | | | | CCmGmUmCm | |
| | | | | | GmAmAmAmG | |
| | | | | | mAmUGUGCm | |
| | | | | | CGmCAAmCG | |
| | | | | | CUCUmGmCC | |
| | | | | | mUmUmCmUG | |
| | | | | | GCAUCG*mU | |
| | | | | | *mU | |
| G028957 | 228 | TRBC1/2 | AGCCCGC | AGCCCGCCCUCAAUGACUC | mA*mG*mC* | chr7: |
| | | | CCUCAAU | CAGAUGUUGUAGCUCCCUG | mCmCGCmCm | 142791888- |
| | | | GACUCCA | AAACCGUUGCUACAAUAAG | CUmCAmAUG | 142791912 |
| | | | GAU | GCCGUCGAAAGAUGUGCCG | ACmUCCAmG | |
| | | | | CAACGCUCUGCCUUCUGGC | AUmGUUGmU | |
| | | | | AUCGUU | mAmGmCUCC | |
| | | | | | CmUmGmAmA | |
| | | | | | mAmCmCGUU | |
| | | | | | mGmCUAmCA | |
| | | | | | AU*AAGmGm | |
| | | | | | CCmGmUmCm | |
| | | | | | GmAmAmAmG | |
| | | | | | mAmUGUGCm | |
| | | | | | CGmCAAmCG | |
| | | | | | CUCUmGmCC | |
| | | | | | mUmUmCmUG | |
| | | | | | GCAUCG*mU | |
| | | | | | *mU | |
| G028958 | 229 | TRBC1/2 | CGGCCAC | CGGCCACCUUCUGGCAGAA | mC*mG*mG* | chr7: |
| | | | CUUCUGG | CCCCCGUUGUAGCUCCCUG | mCmCACmCm | 142791939- |
| | | | CAGAACC | AAACCGUUGCUACAAUAAG | UUmCUmGGC | 142791963 |
| | | | CCC | GCCGUCGAAAGAUGUGCCG | AGmAACCmC | |
| | | | | CAACGCUCUGCCUUCUGGC | CCmGUUGmU | |
| | | | | AUCGUU | mAmGmCUCC | |
| | | | | | CmUmGmAmA | |
| | | | | | mAmCmCGUU | |
| | | | | | mGmCUAmCA | |
| | | | | | AU*AAGmGm | |
| | | | | | CCmGmUmCm | |
| | | | | | GmAmAmAmG | |
| | | | | | mAmUGUGCm | |
| | | | | | CGmCAAmCG | |
| | | | | | CUCUmGmCC | |
| | | | | | mUmUmCmUG | |
| | | | | | GCAUCG*mU | |
| | | | | | *mU | |
| G028959 | 230 | TRBC1/2 | GGGGGUU | GGGGGUUCUGCCAGAAGGU | mG*mG*mG* | chr7: |
| | | | CUGCCAG | GGCCGGUUGUAGCUCCCUG | mGmGUUmCm | 142791939- |
| | | | AAGGUGG | AAACCGUUGCUACAAUAAG | UGmCCmAGA | 142791963 |
| | | | CCG | GCCGUCGAAAGAUGUGCCG | AGmGUGGmC | |
| | | | | CAACGCUCUGCCUUCUGGC | CGmGUUGmU | |
| | | | | AUCGUU | mAmGmCUCC | |
| | | | | | CmUmGmAmA | |
| | | | | | mAmCmCGUU | |
| | | | | | mGmCUAmCA | |
| | | | | | AU*AAGmGm | |
| | | | | | CCmGmUmCm | |
| | | | | | GmAmAmAmG | |
| | | | | | mAmUGUGCm | |
| | | | | | CGmCAAmCG | |
| | | | | | CUCUmGmCC | |
| | | | | | mUmUmCmUG | |
| | | | | | GCAUCG*mU | |
| | | | | | *mU | |
| G028960 | 231 | TRBC1/2 | CGGGGGU | CGGGGGUUCUGCCAGAAGG | mC*mG*mG* | chr7: |
| | | | UCUGCCA | UGGCCGUUGUAGCUCCCUG | mGmGGUmUm | 142791940- |
| | | | GAAGGUG | AAACCGUUGCUACAAUAAG | CUmGCmCAG | 142791964 |
| | | | GCC | GCCGUCGAAAGAUGUGCCG | AAmGGUGmG | |
| | | | | CAACGCUCUGCCUUCUGGC | CCmGUUGmU | |
| | | | | AUCGUU | mAmGmCUCC | |
| | | | | | CmUmGmAmA | |
| | | | | | mAmCmCGUU | |
| | | | | | mGmCUAmCA | |
| | | | | | AU*AAGmGm | |
| | | | | | CCmGmUmCm | |
| | | | | | GmAmAmAmG | |
| | | | | | mAmUGUGCm | |
| | | | | | CGmCAAmCG | |
| | | | | | CUCUmGmCC | |
| | | | | | mUmUmCmUG | |
| | | | | | GCAUCG*mU | |
| | | | | | *mU | |
| G028961 | 232 | TRBC1/2 | CCUUCUG | CCUUCUGGCAGAACCCCCG | mC*mC*mU* | chr7: |
| | | | GCAGAAC | CAACCGUUGUAGCUCCCUG | mUmCUGmGm | 142791945- |
| | | | CCCCGCA | AAACCGUUGCUACAAUAAG | CAmGAmACC | 142791969 |
| | | | ACC | GCCGUCGAAAGAUGUGCCG | CCmCGCAmA | |
| | | | | CAACGCUCUGCCUUCUGGC | CCmGUUGmU | |
| | | | | AUCGUU | mAmGmCUCC | |
| | | | | | CmUmGmAmA | |
| | | | | | mAmCmCGUU | |
| | | | | | mGmCUAmCA | |
| | | | | | AU*AAGmGm | |
| | | | | | CCmGmUmCm | |
| | | | | | GmAmAmAmG | |
| | | | | | mAmUGUGCm | |
| | | | | | CGmCAAmCG | |
| | | | | | CUCUmGmCC | |
| | | | | | mUmUmCmUG | |
| | | | | | GCAUCG*mU | |
| | | | | | *mU | |
| G028962 | 233 | TRBC1/2 | UGGUUGC | UGGUUGCGGGGGUUCUGCC | mU*mG*mG* | chr7: |
| | | | GGGGGUU | AGAAGGUUGUAGCUCCCUG | mUmUGCmGm | 142791946- |
| | | | CUGCCAG | AAACCGUUGCUACAAUAAG | GGmGGmUUC | 142791970 |
| | | | AAG | GCCGUCGAAAGAUGUGCCG | UGmCCAGmA | |
| | | | | CAACGCUCUGCCUUCUGGC | AGmGUUGmU | |
| | | | | AUCGUU | mAmGmCUCC | |
| | | | | | CmUmGmAmA | |
| | | | | | mAmCmCGUU | |
| | | | | | mGmCUAmCA | |
| | | | | | AU*AAGmGm | |
| | | | | | CCmGmUmCm | |
| | | | | | GmAmAmAmG | |
| | | | | | mAmUGUGCm | |
| | | | | | CGmCAAmCG | |
| | | | | | CUCUmGmCC | |
| | | | | | mUmUmCmUG | |
| | | | | | GCAUCG*mU | |
| | | | | | *mU | |
| G028963 | 234 | TRBC1/2 | CUGGGUC | CUGGGUCCACUCGUCAUUC | mC*mU*mG* | chr7: |
| | | | CACUCGU | UCCGAGUUGUAGCUCCCUG | mGmGUCmCm | 142792001- |
| | | | CAUUCUC | AAACCGUUGCUACAAUAAG | ACmUCmGUC | 142792025 |
| | | | CGA | GCCGUCGAAAGAUGUGCCG | AUmUCUCmC | |
| | | | | CAACGCUCUGCCUUCUGGC | GAmGUUGmU | |
| | | | | AUCGUU | mAmGmCUCC | |
| | | | | | CmUmGmAmA | |
| | | | | | mAmCmCGUU | |
| | | | | | mGmCUAmCA | |
| | | | | | AU*AAGmGm | |
| | | | | | CCmGmUmCm | |
| | | | | | GmAmAmAmG | |
| | | | | | mAmUGUGCm | |
| | | | | | CGmCAAmCG | |
| | | | | | CUCUmGmCC | |
| | | | | | mUmUmCmUG | |
| | | | | | GCAUCG*mU | |
| | | | | | *mU | |
| G028964 | 235 | TRBC1/2 | CCUGGGU | CCUGGGUCCACUCGUCAUU | mC*mC*mU* | chr7: |
| | | | CCACUCG | CUCCGGUUGUAGCUCCCUG | mGmGGUmCm | 142792002- |
| | | | UCAUUCU | AAACCGUUGCUACAAUAAG | CAmCUmCGU | 142792026 |
| | | | CCG | GCCGUCGAAAGAUGUGCCG | CAmUUCUmC | |
| | | | | CAACGCUCUGCCUUCUGGC | CGmGUUGmU | |
| | | | | AUCGUU | mAmGmCUCC | |
| | | | | | CmUmGmAmA | |
| | | | | | mAmCmCGUU | |
| | | | | | mGmCUAmCA | |
| | | | | | AU*AAGmGm | |
| | | | | | CCmGmUmCm | |
| | | | | | GmAmAmAmG | |
| | | | | | mAmUGUGCm | |
| | | | | | CGmCAAmCG | |
| | | | | | CUCUmGmCC | |
| | | | | | mUmUmCmUG | |
| | | | | | GCAUCG*mU | |
| | | | | | *mU | |
| G028965 | 236 | TRBC1/2 | GAGAAUG | GAGAAUGACGAGUGGACCC | mG*mA*mG* | chr7: |
| | | | ACGAGUG | AGGAUGUUGUAGCUCCCUG | mAmAUGmAm | 142792004- |
| | | | GACCCAG | AAACCGUUGCUACAAUAAG | CGmAGmUGG | 142792028 |
| | | | GAU | GCCGUCGAAAGAUGUGCCG | ACmCCAGmG | |
| | | | | CAACGCUCUGCCUUCUGGC | AUmGUUGmU | |
| | | | | AUCGUU | mAmGmCUCC | |
| | | | | | CmUmGmAmA | |
| | | | | | mAmCmCGUU | |
| | | | | | mGmCUAmCA | |
| | | | | | AU*AAGmGm | |
| | | | | | CCmGmUmCm | |
| | | | | | GmAmAmAmG | |
| | | | | | mAmUGUGCm | |
| | | | | | CGmCAAmCG | |
| | | | | | CUCUmGmCC | |
| | | | | | mUmUmCmUG | |
| | | | | | GCAUCG*mU | |
| | | | | | *mU | |
| G028966 | 237 | TRBC1/2 | UGACGAG | UGACGAGUGGACCCAGGAU | mU*mG*mA* | chr7: |
| | | | UGGACCC | AGGGCGUUGUAGCUCCCUG | mCmGAGmUm | 142792009- |
| | | | AGGAUAG | AAACCGUUGCUACAAUAAG | GGmACmCCA | 142792033 |
| | | | GGC | GCCGUCGAAAGAUGUGCCG | GGmAUAGmG | |
| | | | | CAACGCUCUGCCUUCUGGC | GCmGUUGmU | |
| | | | | AUCGUU | mAmGmCUCC | |
| | | | | | CmUmGmAmA | |
| | | | | | mAmCmCGUU | |
| | | | | | mGmCUAmCA | |
| | | | | | AU*AAGmGm | |
| | | | | | CCmGmUmCm | |
| | | | | | GmAmAmAmG | |
| | | | | | mAmUGUGCm | |
| | | | | | CGmCAAmCG | |
| | | | | | CUCUmGmCC | |
| | | | | | mUmUmCmUG | |
| | | | | | GCAUCG*mU | |
| | | | | | *mU | |
| G028967 | 238 | TRBC1/2 | GCCCUAU | GCCCUAUCCUGGGUCCACU | mG*mC*mC* | chr7: |
| | | | CCUGGGU | CGUCAGUUGUAGCUCCCUG | mCmUAUmCm | 142792009- |
| | | | CCACUCG | AAACCGUUGCUACAAUAAG | CUmGGmGUC | 142792033 |
| | | | UCA | GCCGUCGAAAGAUGUGCCG | CAmCUCGmU | |
| | | | | CAACGCUCUGCCUUCUGGC | CAmGUUGmU | |
| | | | | AUCGUU | mAmGmCUCC | |
| | | | | | CmUmGmAmA | |
| | | | | | mAmCmCGUU | |
| | | | | | mGmCUAmCA | |
| | | | | | AU*AAGmGm | |
| | | | | | CCmGmUmCm | |
| | | | | | GmAmAmAmG | |
| | | | | | mAmUGUGCm | |
| | | | | | CGmCAAmCG | |
| | | | | | CUCUmGmCC | |
| | | | | | mUmUmCmUG | |
| | | | | | GCAUCG*mU | |
| | | | | | *mU | |
| G028971 | 239 | TRBC1/2 | AGGCCUG | AGGCCUGGGGUAGAGCAGG | mA*mG*mG* | chr7: |
| | | | GGGUAGA | UGAGUGUUGUAGCUCCCUG | mCmCUGmGm | 142792062- |
| | | | GCAGGUG | AAACCGUUGCUACAAUAAG | GGmUAmGAG | 142792086 |
| | | | AGU | GCCGUCGAAAGAUGUGCCG | CAmGGUGmA | |
| | | | | CAACGCUCUGCCUUCUGGC | GUmGUUGmU | |
| | | | | AUCGUU | mAmGmCUCC | |
| | | | | | CmUmGmAmA | |
| | | | | | mAmCmCGUU | |
| | | | | | mGmCUAmCA | |
| | | | | | AU*AAGmGm | |
| | | | | | CCmGmUmCm | |
| | | | | | GmAmAmAmG | |
| | | | | | mAmUGUGCm | |
| | | | | | CGmCAAmCG | |
| | | | | | CUCUmGmCC | |
| | | | | | mUmUmCmUG | |
| | | | | | GCAUCG*mU | |
| | | | | | *mU | |
| G028972 | 240 | TRBC1/2 | AGGCCCC | AGGCCCCACUCACCUGCUC | mA*mG*mG* | chr7: |
| | | | ACUCACC | UACCCGUUGUAGCUCCCUG | mCmCCCmAm | 142792069- |
| | | | UGCUCUA | AAACCGUUGCUACAAUAAG | CUmCAmCCU | 142792093 |
| | | | CCC | GCCGUCGAAAGAUGUGCCG | GCmUCUAmC | |
| | | | | CAACGCUCUGCCUUCUGGC | CCmGUUGmU | |
| | | | | AUCGUU | mAmGmCUCC | |
| | | | | | CmUmGmAmA | |
| | | | | | mAmCmCGUU | |
| | | | | | mGmCUAmCA | |
| | | | | | AU*AAGmGm | |
| | | | | | CCmGmUmCm | |
| | | | | | GmAmAmAmG | |
| | | | | | mAmUGUGCm | |
| | | | | | CGmCAAmCG | |
| | | | | | CUCUmGmCC | |
| | | | | | mUmUmCmUG | |
| | | | | | GCAUCG*mU | |
| | | | | | *mU | |
| G028973 | 241 | TRBC1/2 | GAGCAGG | GAGCAGGUGAGUGGGGCCU | mG*mA*mG* | chr7: |
| | | | UGAGUGG | GGGGAGUUGUAGCUCCCUG | mCmAGGmUm | 142792074- |
| | | | GGCCUGG | AAACCGUUGCUACAAUAAG | GAmGUmGGG | 142792098 |
| | | | GGA | GCCGUCGAAAGAUGUGCCG | GCmCUGGmG | |
| | | | | CAACGCUCUGCCUUCUGGC | GAmGUUGmU | |
| | | | | AUCGUU | mAmGmCUCC | |
| | | | | | CmUmGmAmA | |
| | | | | | mAmCmCGUU | |
| | | | | | mGmCUAmCA | |
| | | | | | AU*AAGmGm | |
| | | | | | CCmGmUmCm | |
| | | | | | GmAmAmAmG | |
| | | | | | mAmUGUGCm | |
| | | | | | CGmCAAmCG | |
| | | | | | CUCUmGmCC | |
| | | | | | mUmUmCmUG | |
| | | | | | GCAUCG*mU | |
| | | | | | *mU | |
| G028974 | 242 | TRBC1/2 | UCCCCAG | UCCCCAGGCCCCACUCACC | mU*mC*mC* | chr7: |
| | | | GCCCCAC | UGCUCGUUGUAGCUCCCUG | mCmCAGmGm | 142792074- |
| | | | UCACCUG | AAACCGUUGCUACAAUAAG | CCmCCmACU | 142792098 |
| | | | CUC | GCCGUCGAAAGAUGUGCCG | CAmCCUGmC | |
| | | | | CAACGCUCUGCCUUCUGGC | UCmGUUGmU | |
| | | | | AUCGUU | mAmGmCUCC | |
| | | | | | CmUmGmAmA | |
| | | | | | mAmCmCGUU | |
| | | | | | mGmCUAmCA | |
| | | | | | AU*AAGmGm | |
| | | | | | CCmGmUmCm | |
| | | | | | GmAmAmAmG | |
| | | | | | mAmUGUGCm | |
| | | | | | CGmCAAmCG | |
| | | | | | CUCUmGmCC | |
| | | | | | mUmUmCmUG | |
| | | | | | GCAUCG*mU | |
| | | | | | *mU | |
| G028975 | 243 | TRBC1/2 | CUCCCCA | CUCCCCAGGCCCCACUCAC | mC*mU*mC* | chr7: |
| | | | GGCCCCA | CUGCUGUUGUAGCUCCCUG | mCmCCAmGm | 142792075- |
| | | | CUCACCU | AAACCGUUGCUACAAUAAG | GCmCCmCAC | 142792099 |
| | | | GCU | GCCGUCGAAAGAUGUGCCG | UCmACCUmG | |
| | | | | CAACGCUCUGCCUUCUGGC | CUmGUUGmU | |
| | | | | AUCGUU | mAmGmCUCC | |
| | | | | | CmUmGmAmA | |
| | | | | | mAmCmCGUU | |
| | | | | | mGmCUAmCA | |
| | | | | | AU*AAGmGm | |
| | | | | | CCmGmUmCm | |
| | | | | | GmAmAmAmG | |
| | | | | | mAmUGUGCm | |
| | | | | | CGmCAAmCG | |
| | | | | | CUCUmGmCC | |
| | | | | | mUmUmCmUG | |
| | | | | | GCAUCG*mU | |
| | | | | | *mU | |
| G028976 | 244 | TRBC1/2 | UCUCCCC | UCUCCCCAGGCCCCACUCA | mU*mC*mU* | chr7: |
| | | | AGGCCCC | CCUGCGUUGUAGCUCCCUG | mCmCCCmAm | 142792076- |
| | | | ACUCACC | AAACCGUUGCUACAAUAAG | GGmCCmCCA | 142792100 |
| | | | UGC | GCCGUCGAAAGAUGUGCCG | CUmCACCmU | |
| | | | | CAACGCUCUGCCUUCUGGC | GCmGUUGmU | |
| | | | | AUCGUU | mAmGmCUCC | |
| | | | | | CmUmGmAmA | |
| | | | | | mAmCmCGUU | |
| | | | | | mGmCUAmCA | |
| | | | | | AU*AAGmGm | |
| | | | | | CCmGmUmCm | |
| | | | | | GmAmAmAmG | |
| | | | | | mAmUGUGCm | |
| | | | | | CGmCAAmCG | |
| | | | | | CUCUmGmCC | |
| | | | | | mUmUmCmUG | |
| | | | | | GCAUCG*mU | |
| | | | | | *mU | |
| G028988 | 245 | TRBC1/2 | ACCAGCA | ACCAGCAAGGGGUCCUGUC | mA*mC*mC* | chr7: |
| | | | AGGGGUC | UGCCAGUUGUAGCUCCCUG | mAmGCAmAm | 142792697- |
| | | | CUGUCUG | AAACCGUUGCUACAAUAAG | GGmGGmUCC | 142792721 |
| | | | CCA | GCCGUCGAAAGAUGUGCCG | UGmUCUGmC | |
| | | | | CAACGCUCUGCCUUCUGGC | CAmGUUGmU | |
| | | | | AUCGUU | mAmGmCUCC | |
| | | | | | CmUmGmAmA | |
| | | | | | mAmCmCGUU | |
| | | | | | mGmCUAmCA | |
| | | | | | AU*AAGmGm | |
| | | | | | CCmGmUmCm | |
| | | | | | GmAmAmAmG | |
| | | | | | mAmUGUGCm | |
| | | | | | CGmCAAmCG | |
| | | | | | CUCUmGmCC | |
| | | | | | mUmUmCmUG | |
| | | | | | GCAUCG*mU | |
| | | | | | *mU | |
| G027904 | 246 | TRBC2 | CCACACC | CCACACCCAAAAGGCCACA | mC*mC*mA* | chr7: |
| | | | CAAAAGG | CGUUUUAGAGCUAGAAAUA | CACCCAAAA | 142801104- |
| | | | CCACAC | GCAAGUUAAAAUAAGGCUA | GGCCACACG | 142801124 |
| | | | | GUCCGUUAUCACGAAAGGG | UUUUAGAmG | |
| | | | | CACCGAGUCGGUGCU | mCmUmAmGm | |
| | | | | | AmAmAmUmA | |
| | | | | | mGmCAAGUU | |
| | | | | | AAAAUAAGG | |
| | | | | | CUAGUCCGU | |
| | | | | | UAUCACGAA | |
| | | | | | AGGGCACCG | |
| | | | | | AGUCGGmU* | |
| | | | | | mG*mC*mU | |
| G028994 | 247 | TRBC2 | CACAGAC | CACAGACCCGCAGCCCCUC | mC*mA*mC* | chr7: |
| | | | CCGCAGC | AAGGAGUUGUAGCUCCCUG | mAmGACmCm | 142801209- |
| | | | CCCUCAA | AAACCGUUGCUACAAUAAG | CGmCAmGCC | 142801233 |
| | | | GGA | GCCGUCGAAAGAUGUGCCG | CCmUCAAmG | |
| | | | | CAACGCUCUGCCUUCUGGC | GAmGUUGmU | |
| | | | | AUCGUU | mAmGmCUCC | |
| | | | | | CmUmGmAmA | |
| | | | | | mAmCmCGUU | |
| | | | | | mGmCUAmCA | |
| | | | | | AU*AAGmGm | |
| | | | | | CCmGmUmCm | |
| | | | | | GmAmAmAmG | |
| | | | | | mAmUGUGCm | |
| | | | | | CGmCAAmCG | |
| | | | | | CUCUmGmCC | |
| | | | | | mUmUmCmUG | |
| | | | | | GCAUCG*mU | |
| | | | | | *mU | |
| G028995 | 248 | TRBC2 | ACAGACC | ACAGACCCGCAGCCCCUCA | mA*mC*mA* | chr7: |
| | | | CGCAGCC | AGGAGGUUGUAGCUCCCUG | mGmACCmCm | 142801210- |
| | | | CCUCAAG | AAACCGUUGCUACAAUAAG | GCmAGmCCC | 142801234 |
| | | | GAG | GCCGUCGAAAGAUGUGCCG | CUmCAAGmG | |
| | | | | CAACGCUCUGCCUUCUGGC | AGmGUUGmU | |
| | | | | AUCGUU | mAmGmCUCC | |
| | | | | | CmUmGmAmA | |
| | | | | | mAmCmCGUU | |
| | | | | | mGmCUAmCA | |
| | | | | | AU*AAGmGm | |
| | | | | | CCmGmUmCm | |
| | | | | | GmAmAmAmG | |
| | | | | | mAmUGUGCm | |
| | | | | | CGmCAAmCG | |
| | | | | | CUCUmGmCC | |
| | | | | | mUmUmCmUG | |
| | | | | | GCAUCG*mU | |
| | | | | | *mU | |
| G028996 | 249 | TRBC2 | UGGACCC | UGGACCCAGGAUAGGGCCA | mU*mG*mG* | chr7: |
| | | | AGGAUAG | AACCUGUUGUAGCUCCCUG | mAmCCCmAm | 142801363- |
| | | | GGCCAAA | AAACCGUUGCUACAAUAAG | GGmAUmAGG | 142801387 |
| | | | CCU | GCCGUCGAAAGAUGUGCCG | GCmCAAAmC | |
| | | | | CAACGCUCUGCCUUCUGGC | CUmGUUGmU | |
| | | | | AUCGUU | mAmGmCUCC | |
| | | | | | CmUmGmAmA | |
| | | | | | mAmCmCGUU | |
| | | | | | mGmCUAmCA | |
| | | | | | AU*AAGmGm | |
| | | | | | CCmGmUmCm | |
| | | | | | GmAmAmAmG | |
| | | | | | mAmUGUGCm | |
| | | | | | CGmCAAmCG | |
| | | | | | CUCUmGmCC | |
| | | | | | mUmUmCmUG | |
| | | | | | GCAUCG*mU | |
| | | | | | *mU | |
| G028997 | 250 | TRBC2 | GGACCCA | GGACCCAGGAUAGGGCCAA | mG*mG*mA* | chr7: |
| | | | GGAUAGG | ACCUGGUUGUAGCUCCCUG | mCmCCAmGm | 142801364- |
| | | | GCCAAAC | AAACCGUUGCUACAAUAAG | GAmUAmGGG | 142801388 |
| | | | CUG | GCCGUCGAAAGAUGUGCCG | CCmAAACmC | |
| | | | | CAACGCUCUGCCUUCUGGC | UGmGUUGmU | |
| | | | | AUCGUU | mAmGmCUCC | |
| | | | | | CmUmGmAmA | |
| | | | | | mAmCmCGUU | |
| | | | | | mGmCUAmCA | |
| | | | | | AU*AAGmGm | |
| | | | | | CCmGmUmCm | |
| | | | | | GmAmAmAmG | |
| | | | | | mAmUGUGCm | |
| | | | | | CGmCAAmCG | |
| | | | | | CUCUmGmCC | |
| | | | | | mUmUmCmUG | |
| | | | | | GCAUCG*mU | |
| | | | | | *mU | |
| G028998 | 251 | TRBC2 | GCCAAAC | GCCAAACCUGUCACCCAGA | mG*mC*mC* | chr7: |
| | | | CUGUCAC | UCGUCGUUGUAGCUCCCUG | mAmAACmCm | 142801378- |
| | | | CCAGAUC | AAACCGUUGCUACAAUAAG | UGmUCmACC | 142801402 |
| | | | GUC | GCCGUCGAAAGAUGUGCCG | CAmGAUCmG | |
| | | | | CAACGCUCUGCCUUCUGGC | UCmGUUGmU | |
| | | | | AUCGUU | mAmGmCUCC | |
| | | | | | CmUmGmAmA | |
| | | | | | mAmCmCGUU | |
| | | | | | mGmCUAmCA | |
| | | | | | AU*AAGmGm | |
| | | | | | CCmGmUmCm | |
| | | | | | GmAmAmAmG | |
| | | | | | mAmUGUGCm | |
| | | | | | CGmCAAmCG | |
| | | | | | CUCUmGmCC | |
| | | | | | mUmUmCmUG | |
| | | | | | GCAUCG*mU | |
| | | | | | *mU | |
| G028999 | 252 | TRBC2 | CCUGUCA | CCUGUCACCCAGAUCGUCA | mC*mC*mU* | chr7: |
| | | | CCCAGAU | GCGCCGUUGUAGCUCCCUG | mGmUCAmCm | 142801384- |
| | | | CGUCAGC | AAACCGUUGCUACAAUAAG | CCmAGmAUC | 142801408 |
| | | | GCC | GCCGUCGAAAGAUGUGCCG | GUmCAGCmG | |
| | | | | CAACGCUCUGCCUUCUGGC | CCmGUUGmU | |
| | | | | AUCGUU | mAmGmCUCC | |
| | | | | | CmUmGmAmA | |
| | | | | | mAmCmCGUU | |
| | | | | | mGmCUAmCA | |
| | | | | | AU*AAGmGm | |
| | | | | | CCmGmUmCm | |
| | | | | | GmAmAmAmG | |
| | | | | | mAmUGUGCm | |
| | | | | | CGmCAAmCG | |
| | | | | | CUCUmGmCC | |
| | | | | | mUmUmCmUG | |
| | | | | | GCAUCG*mU | |
| | | | | | *mU | |
| G029000 | 253 | TRBC2 | CCCUGUU | CCCUGUUUUCUUUCAGACU | mC*mC*mC* | chr7: |
| | | | UUCUUUC | GUGGCGUUGUAGCUCCCUG | mUmGUUmUm | 142801927- |
| | | | AGACUGU | AAACCGUUGCUACAAUAAG | UCmUUmUCA | 142801951 |
| | | | GGC | GCCGUCGAAAGAUGUGCCG | GAmCUGUmG | |
| | | | | CAACGCUCUGCCUUCUGGC | GCmGUUGmU | |
| | | | | AUCGUU | mAmGmCUCC | |
| | | | | | CmUmGmAmA | |
| | | | | | mAmCmCGUU | |
| | | | | | mGmCUAmCA | |
| | | | | | AU*AAGmGm | |
| | | | | | CCmGmUmCm | |
| | | | | | GmAmAmAmG | |
| | | | | | mAmUGUGCm | |
| | | | | | CGmCAAmCG | |
| | | | | | CUCUmGmCC | |
| | | | | | mUmUmCmUG | |
| | | | | | GCAUCG*mU | |
| | | | | | *mU | |
| G029001 | 254 | TRBC2 | UGUUUUC | UGUUUUCUUUCAGACUGUG | mU*mG*mU* | chr7: |
| | | | UUUCAGA | GCUUCGUUGUAGCUCCCUG | mUmUUCmUm | 142801930- |
| | | | CUGUGGC | AAACCGUUGCUACAAUAAG | UUmCAmGAC | 142801954 |
| | | | UUC | GCCGUCGAAAGAUGUGCCG | UGmUGGCmU | |
| | | | | CAACGCUCUGCCUUCUGGC | UCmGUUGmU | |
| | | | | AUCGUU | mAmGmCUCC | |
| | | | | | CmUmGmAmA | |
| | | | | | mAmCmCGUU | |
| | | | | | mGmCUAmCA | |
| | | | | | AU*AAGmGm | |
| | | | | | CCmGmUmCm | |
| | | | | | GmAmAmAmG | |
| | | | | | mAmUGUGCm | |
| | | | | | CGmCAAmCG | |
| | | | | | CUCUmGmCC | |
| | | | | | mUmUmCmUG | |
| | | | | | GCAUCG*mU | |
| | | | | | *mU | |
| G029002 | 255 | TRBC2 | UGGCUUC | UGGCUUCACCUCCGGUAAG | mU*mG*mG* | chr7: |
| | | | ACCUCCG | UGAGUGUUGUAGCUCCCUG | mCmUUCmAm | 142801947- |
| | | | GUAAGUG | AAACCGUUGCUACAAUAAG | CCmUCmCGG | 142801971 |
| | | | AGU | GCCGUCGAAAGAUGUGCCG | UAmAGUGmA | |
| | | | | CAACGCUCUGCCUUCUGGC | GUmGUUGmU | |
| | | | | AUCGUU | mAmGmCUCC | |
| | | | | | CmUmGmAmA | |
| | | | | | mAmCmCGUU | |
| | | | | | mGmCUAmCA | |
| | | | | | AU*AAGmGm | |
| | | | | | CCmGmUmCm | |
| | | | | | GmAmAmAmG | |
| | | | | | mAmUGUGCm | |
| | | | | | CGmCAAmCG | |
| | | | | | CUCUmGmCC | |
| | | | | | mUmUmCmUG | |
| | | | | | GCAUCG*mU | |
| | | | | | *mU | |
| G029003 | 256 | TRBC2 | AGGAGAG | AGGAGAGACUCACUUACCG | mA*mG*mG* | chr7: |
| | | | ACUCACU | GAGGUGUUGUAGCUCCCUG | mAmGAGmAm | 142801954- |
| | | | UACCGGA | AAACCGUUGCUACAAUAAG | CUmCAmCUU | 142801978 |
| | | | GGU | GCCGUCGAAAGAUGUGCCG | ACmCGGAmG | |
| | | | | CAACGCUCUGCCUUCUGGC | GUmGUUGmU | |
| | | | | AUCGUU | mAmGmCUCC | |
| | | | | | CmUmGmAmA | |
| | | | | | mAmCmCGUU | |
| | | | | | mGmCUAmCA | |
| | | | | | AU*AAGmGm | |
| | | | | | CCmGmUmCm | |
| | | | | | GmAmAmAmG | |
| | | | | | mAmUGUGCm | |
| | | | | | CGmCAAmCG | |
| | | | | | CUCUmGmCC | |
| | | | | | mUmUmCmUG | |
| | | | | | GCAUCG*mU | |
| | | | | | *mU | |
| G029004 | 257 | TRBC2 | CUGUUGA | CUGUUGACAAGAACAGAGC | mC*mU*mG* | chr7: |
| | | | CAAGAAC | AUGUAGUUGUAGCUCCCUG | mUmUGAmCm | 142802080- |
| | | | AGAGCAU | AAACCGUUGCUACAAUAAG | AAmGAmACA | 142802104 |
| | | | GUA | GCCGUCGAAAGAUGUGCCG | GAmGCAUmG | |
| | | | | CAACGCUCUGCCUUCUGGC | UAmGUUGmU | |
| | | | | AUCGUU | mAmGmCUCC | |
| | | | | | CmUmGmAmA | |
| | | | | | mAmCmCGUU | |
| | | | | | mGmCUAmCA | |
| | | | | | AU*AAGmGm | |
| | | | | | CCmGmUmCm | |
| | | | | | GmAmAmAmG | |
| | | | | | mAmUGUGCm | |
| | | | | | CGmCAAmCG | |
| | | | | | CUCUmGmCC | |
| | | | | | mUmUmCmUG | |
| | | | | | GCAUCG*mU | |
| | | | | | *mU | |
| G029005 | 258 | TRBC2 | UGUCAAC | UGUCAACAGAGUCUUACCA | mU*mG*mU* | chr7: |
| | | | AGAGUCU | GCAAGGUUGUAGCUCCCUG | mCmAACmAm | 142802095- |
| | | | UACCAGC | AAACCGUUGCUACAAUAAG | GAmGUmCUU | 142802119 |
| | | | AAG | GCCGUCGAAAGAUGUGCCG | ACmCAGCmA | |
| | | | | CAACGCUCUGCCUUCUGGC | AGmGUUGmU | |
| | | | | AUCGUU | mAmGmCUCC | |
| | | | | | CmUmGmAmA | |
| | | | | | mAmCmCGUU | |
| | | | | | mGmCUAmCA | |
| | | | | | AU*AAGmGm | |
| | | | | | CCmGmUmCm | |
| | | | | | GmAmAmAmG | |
| | | | | | mAmUGUGCm | |
| | | | | | CGmCAAmCG | |
| | | | | | CUCUmGmCC | |
| | | | | | mUmUmCmUG | |
| | | | | | GCAUCG*mU | |
| | | | | | *mU | |
| G029006 | 259 | TRBC2 | GAGUCUU | GAGUCUUACCAGCAAGGGG | mG*mA*mG* | chr7: |
| | | | ACCAGCA | UCCUGGUUGUAGCUCCCUG | mUmCUUmAm | 142802103- |
| | | | AGGGGUC | AAACCGUUGCUACAAUAAG | CCmAGmCAA | 142802127 |
| | | | CUG | GCCGUCGAAAGAUGUGCCG | GGmGGUCmC | |
| | | | | CAACGCUCUGCCUUCUGGC | UGmGUUGmU | |
| | | | | AUCGUU | mAmGmCUCC | |
| | | | | | CmUmGmAmA | |
| | | | | | mAmCmCGUU | |
| | | | | | mGmCUAmCA | |
| | | | | | AU*AAGmGm | |
| | | | | | CCmGmUmCm | |
| | | | | | GmAmAmAmG | |
| | | | | | mAmUGUGCm | |
| | | | | | CGmCAAmCG | |
| | | | | | CUCUmGmCC | |
| | | | | | mUmUmCmUG | |
| | | | | | GCAUCG*mU | |
| | | | | | *mU | |
| G029007 | 260 | TRBC2 | UCUUACC | UCUUACCAGCAAGGGGUCC | mU*mC*mU* | chr7: |
| | | | AGCAAGG | UGUCUGUUGUAGCUCCCUG | mUmACCmAm | 142802106- |
| | | | GGUCCUG | AAACCGUUGCUACAAUAAG | GCmAAmGGG | 142802130 |
| | | | UCU | GCCGUCGAAAGAUGUGCCG | GUmCCUGmU | |
| | | | | CAACGCUCUGCCUUCUGGC | CUmGUUGmU | |
| | | | | AUCGUU | mAmGmCUCC | |
| | | | | | CmUmGmAmA | |
| | | | | | mAmCmCGUU | |
| | | | | | mGmCUAmCA | |
| | | | | | AU*AAGmGm | |
| | | | | | CCmGmUmCm | |
| | | | | | GmAmAmAmG | |
| | | | | | mAmUGUGCm | |
| | | | | | CGmCAAmCG | |
| | | | | | CUCUmGmCC | |
| | | | | | mUmUmCmUG | |
| | | | | | GCAUCG*mU | |
| | | | | | *mU | |
| G029008 | 261 | TRBC2 | ACCAUGG | ACCAUGGCCAUCAGCACGA | mA*mC*mC* | chr7: |
| | | | CCAUCAG | GGGCAGUUGUAGCUCCCUG | mAmUGGmCm | 142802189- |
| | | | CACGAGG | AAACCGUUGCUACAAUAAG | CAmUCmAGC | 142802213 |
| | | | GCA | GCCGUCGAAAGAUGUGCCG | ACmGAGGmG | |
| | | | | CAACGCUCUGCCUUCUGGC | CAmGUUGmU | |
| | | | | AUCGUU | mAmGmCUCC | |
| | | | | | CmUmGmAmA | |
| | | | | | mAmCmCGUU | |
| | | | | | mGmCUAmCA | |
| | | | | | AU*AAGmGm | |
| | | | | | CCmGmUmCm | |
| | | | | | GmAmAmAmG | |
| | | | | | mAmUGUGCm | |
| | | | | | CGmCAAmCG | |
| | | | | | CUCUmGmCC | |
| | | | | | mUmUmCmUG | |
| | | | | | GCAUCG*mU | |
| | | | | | *mU | |
| G029009 | 262 | TRBC2 | UCUCUUC | UCUCUUCCACAGGUCAAGA | mU*mC*mU* | chr7: |
| | | | CACAGGU | GAAAGGUUGUAGCUCCCUG | mCmUUCmCm | 142802490- |
| | | | CAAGAGA | AAACCGUUGCUACAAUAAG | ACmAGmGUC | 142802514 |
| | | | AAG | GCCGUCGAAAGAUGUGCCG | AAmGAGAmA | |
| | | | | CAACGCUCUGCCUUCUGGC | AGmGUUGmU | |
| | | | | AUCGUU | mAmGmCUCC | |
| | | | | | CmUmGmAmA | |
| | | | | | mAmCmCGUU | |
| | | | | | mGmCUAmCA | |
| | | | | | AU*AAGmGm | |
| | | | | | CCmGmUmCm | |
| | | | | | GmAmAmAmG | |
| | | | | | mAmUGUGCm | |
| | | | | | CGmCAAmCG | |
| | | | | | CUCUmGmCC | |
| | | | | | mUmUmCmUG | |
| | | | | | GCAUCG*mU | |
| | | | | | *mU | |
| G029010 | 263 | TRBC2 | UCAAGAG | UCAAGAGAAAGGAUUCCAG | mU*mC*mA* | chr7: |
| | | | AAAGGAU | AGGCUGUUGUAGCUCCCUG | mAmGAGmAm | 142802503- |
| | | | UCCAGAG | AAACCGUUGCUACAAUAAG | AAmGGmAUU | 142802527 |
| | | | GCU | GCCGUCGAAAGAUGUGCCG | CCmAGAGmG | |
| | | | | CAACGCUCUGCCUUCUGGC | CUmGUUGmU | |
| | | | | AUCGUU | mAmGmCUCC | |
| | | | | | CmUmGmAmA | |
| | | | | | mAmCmCGUU | |
| | | | | | mGmCUAmCA | |
| | | | | | AU*AAGmGm | |
| | | | | | CCmGmUmCm | |
| | | | | | GmAmAmAmG | |
| | | | | | mAmUGUGCm | |
| | | | | | CGmCAAmCG | |
| | | | | | CUCUmGmCC | |
| | | | | | mUmUmCmUG | |
| | | | | | GCAUCG*mU | |
| | | | | | *mU | |
| G029011 | 264 | TRBC2 | AGCUAGC | AGCUAGCCUCUGGAAUCCU | mA*mG*mC* | chr7: |
| | | | CUCUGGA | UUCUCGUUGUAGCUCCCUG | mUmAGCmCm | 142802507- |
| | | | AUCCUUU | AAACCGUUGCUACAAUAAG | UCmUGmGAA | 142802531 |
| | | | CUC | GCCGUCGAAAGAUGUGCCG | UCmCUUUmC | |
| | | | | CAACGCUCUGCCUUCUGGC | UCmGUUGmU | |
| | | | | AUCGUU | mAmGmCUCC | |
| | | | | | CmUmGmAmA | |
| | | | | | mAmCmCGUU | |
| | | | | | mGmCUAmCA | |
| | | | | | AU*AAGmGm | |
| | | | | | CCmGmUmCm | |
| | | | | | GmAmAmAmG | |
| | | | | | mAmUGUGCm | |
| | | | | | CGmCAAmCG | |
| | | | | | CUCUmGmCC | |
| | | | | | mUmUmCmUG | |
| | | | | | GCAUCG*mU | |
| | | | | | *mU | |
| G029012 | 265 | TRBC2 | GGAUGGU | GGAUGGUUUUGGAGCUAGC | mG*mG*mA* | chr7: |
| | | | UUUGGAG | CUCUGGUUGUAGCUCCCUG | mUmGGUmUm | 142802519- |
| | | | CUAGCCU | AAACCGUUGCUACAAUAAG | UUmGGmAGC | 142802543 |
| | | | CUG | GCCGUCGAAAGAUGUGCCG | UAmGCCUmC | |
| | | | | CAACGCUCUGCCUUCUGGC | UGmGUUGmU | |
| | | | | AUCGUU | mAmGmCUCC | |
| | | | | | CmUmGmAmA | |
| | | | | | mAmCmCGUU | |
| | | | | | mGmCUAmCA | |
| | | | | | AU*AAGmGm | |
| | | | | | CCmGmUmCm | |
| | | | | | GmAmAmAmG | |
| | | | | | mAmUGUGCm | |
| | | | | | CGmCAAmCG | |
| | | | | | CUCUmGmCC | |
| | | | | | mUmUmCmUG | |
| | | | | | GCAUCG*mU | |
| | | | | | *mU | |

The terms “mA,” “mC,” “mU,” or “mG” may be used to denote a nucleotide that has been modified with 2′—O-Me. The terms A*, C*, U*, or G* may be used to denote a nucleotide that is linked to the next (e.g., 3′) nucleotide with a phosphorothioate (PS) bond.

In some embodiments, the TRBC guide RNA comprises a guide sequence selected from SEQ ID NOs: 201-265. In some embodiments, the TRBC guide RNA comprises a guide sequence that is at least 19, 20, 21, 22, 23, or 24 contiguous nucleotides of a sequence selected from SEQ ID NOs: 201-265. In some embodiments, the TRBC guide RNA comprises a guide sequence that is at least 95%, 90%, or 85% identical to a sequence selected from SEQ ID NOs: 201-265. In some embodiments, the TRBC guide RNA comprises a guide sequence that is at least 95% identical to a sequence selected from SEQ ID NOs: 201-265.

In some embodiments, the TRBC guide RNA comprises a guide sequence that comprises at least 10 contiguous nucleotides±10 nucleotides of a genomic coordinate listed in Table 3. As used herein, at least 10 contiguous nucleotides±10 nucleotides of a genomic coordinate means, for example, at least 10 contiguous nucleotides within the genomic coordinates wherein the genomic coordinates include 10 nucleotides in the 5′ direction and 10 nucleotides in the 3′ direction from the ranges listed in Table 3. For example, a TRBC guide RNA may comprise 10 contiguous nucleotides within the genomic coordinates chosen from: (a) chr7:142792690-142792714 and chr7:142792693-142792717; or (b) chr7:142791761-142791785; chr7:142791820-142791844; and chr7:142791939-142791963; chr7:142791756-142791780; or (c) chr7:142801104-142801124; chr7:142802103-142802127; and chr7:142802106-142802130; including the boundary nucleotides of these ranges. In some embodiments, the TRBC guide RNA comprises a guide sequence that is at least 19, 20, 21, 22, 23, or 24 contiguous nucleotides of a sequence that comprises 10 contiguous nucleotides±10 nucleotides of a genomic coordinate listed in Table 3. In some embodiments, the TRBC guide RNA comprises a guide sequence that is at least 95%, 90%, or 85% identical to a sequence selected from a sequence that is 19, 20, 21, 22, 23, or 24 contiguous nucleotides of a sequence that comprises 10 contiguous nucleotides±10 nucleotides of a genomic coordinate listed in Table 3.

In some embodiments, the TRBC guide RNA comprises a guide sequence that comprises at least 15 contiguous nucleotides±10 nucleotides of a genomic coordinate listed in Table 3. In some embodiments, the TRAC guide RNA comprises a guide sequence that comprises at least 24 contiguous nucleotides±10 nucleotides of a genomic coordinate listed in Table 3.

In some embodiments, the TRBC1 guide RNA comprises SEQ ID NO: 201. In some embodiments, the TRBC1 guide RNA comprises SEQ ID NO: 202. In some embodiments, the TRBC1 guide RNA comprises SEQ ID NO: 203. In some embodiments, the TRBC1 guide RNA comprises SEQ ID NO: 204. In some embodiments, the TRBC1 guide RNA comprises SEQ ID NO: 205. In some embodiments, the TRBC1 guide RNA comprises SEQ ID NO: 206. In some embodiments, the TRBC1 guide RNA comprises SEQ ID NO: 207. In some embodiments, the TRBC1 guide RNA comprises SEQ ID NO: 208. In some embodiments, the TRBC1 guide RNA comprises SEQ ID NO: 209. In some embodiments, the TRBC1 guide RNA comprises SEQ ID NO: 210. In some embodiments, the TRBC1 guide RNA comprises SEQ ID NO: 211. In some embodiments, the TRBC1 guide RNA comprises SEQ ID NO: 212. In some embodiments, the TRBC1 guide RNA comprises SEQ ID NO: 213. In some embodiments, the TRBC1 guide RNA comprises SEQ ID NO: 214. In some embodiments, the TRBC1 guide RNA comprises SEQ ID NO: 215. In some embodiments, the TRBC1 guide RNA comprises SEQ ID NO: 216. In some embodiments, the TRBC1 guide RNA comprises SEQ ID NO: 217. In some embodiments, the TRBC1 guide RNA comprises SEQ ID NO: 218. In some embodiments, the TRBC1 guide RNA comprises SEQ ID NO: 219. In some embodiments, the TRBC1 guide RNA comprises SEQ ID NO: 220. In some embodiments, the TRBC1 guide RNA comprises SEQ ID NO: 221.

In some embodiments, the TRBC guide RNA comprises SEQ ID NO. 222. In some embodiments, the TRBC guide RNA comprises SEQ ID NO. 223. In some embodiments, the TRBC guide RNA comprises SEQ ID NO. 224. In some embodiments, the TRBC guide RNA comprises SEQ ID NO. 225. In some embodiments, the TRBC guide RNA comprises SEQ ID NO. 226. In some embodiments, the TRBC guide RNA comprises SEQ ID NO. 227. In some embodiments, the TRBC guide RNA comprises SEQ ID NO. 228. In some embodiments, the TRBC guide RNA comprises SEQ ID NO. 229. In some embodiments, the TRBC guide RNA comprises SEQ ID NO. 230. In some embodiments, the TRBC guide RNA comprises SEQ ID NO. 231. In some embodiments, the TRBC guide RNA comprises SEQ ID NO. 232. In some embodiments, the TRBC guide RNA comprises SEQ ID NO. 233. In some embodiments, the TRBC guide RNA comprises SEQ ID NO. 234. In some embodiments, the TRBC guide RNA comprises SEQ ID NO. 235. In some embodiments, the TRBC guide RNA comprises SEQ ID NO. 236. In some embodiments, the TRBC guide RNA comprises SEQ ID NO. 237. In some embodiments, the TRBC guide RNA comprises SEQ ID NO. 238. In some embodiments, the TRBC guide RNA comprises SEQ ID NO. 239. In some embodiments, the TRBC guide RNA comprises SEQ ID NO. 240. In some embodiments, the TRBC guide RNA comprises SEQ ID NO. 241. In some embodiments, the TRBC guide RNA comprises SEQ ID NO. 242. In some embodiments, the TRBC guide RNA comprises SEQ ID NO. 243. In some embodiments, the TRBC guide RNA comprises SEQ ID NO. 244. In some embodiments, the TRBC guide RNA comprises SEQ ID NO. 245.

In some embodiments, the TRBC2 guide RNA comprises SEQ ID NO. 246. In some embodiments, the TRBC2 guide RNA comprises SEQ ID NO. 247. In some embodiments, the TRBC2 guide RNA comprises SEQ ID NO. 248. In some embodiments, the TRBC2 guide RNA comprises SEQ ID NO. 249. In some embodiments, the TRBC2 guide RNA comprises SEQ ID NO. 250. In some embodiments, the TRBC2 guide RNA comprises SEQ ID NO. 251. In some embodiments, the TRBC2 guide RNA comprises SEQ ID NO. 252. In some embodiments, the TRBC2 guide RNA comprises SEQ ID NO. 253. In some embodiments, the TRBC2 guide RNA comprises SEQ ID NO. 254. In some embodiments, the TRBC2 guide RNA comprises SEQ ID NO. 255. In some embodiments, the TRBC2 guide RNA comprises SEQ ID NO. 256. In some embodiments, the TRBC2 guide RNA comprises SEQ ID NO. 257. In some embodiments, the TRBC2 guide RNA comprises SEQ ID NO. 258. In some embodiments, the TRBC2 guide RNA comprises SEQ ID NO. 259. In some embodiments, the TRBC2 guide RNA comprises SEQ ID NO. 260. In some embodiments, the TRBC2 guide RNA comprises SEQ ID NO. 261. In some embodiments, the TRBC2 guide RNA comprises SEQ ID NO. 262. In some embodiments, the TRBC2 guide RNA comprises SEQ ID NO. 263. In some embodiments, the TRBC2 guide RNA comprises SEQ ID NO. 264. In some embodiments, the TRBC2 guide RNA comprises SEQ ID NO. 265.

In some embodiments, the TRBC1 guide RNA comprises a guide sequence of any one of: SEQ ID NOs: 215-216.

In some embodiments, the TRBC guide RNA comprises a nucleotide chosen form SEQ ID NOs: 223-224 and 229-230.

In some embodiments, the TRBC2 guide RNA comprises a guide sequence of any one of SEQ ID NOs: 246 and 259-260.

In some embodiments, the TRBC guide RNA comprises a guide sequence of any one of SEQ ID NOs: 215, 259, and 260.

In some embodiments, the TRBC guide RNA comprises a sequence listed in Table 3. In some embodiments, the TRBC guide RNA comprises a sequence of any one of SEQ ID NOs: 201-265. In some embodiments, the TRBC guide RNA comprises a sequence of SEQ ID NO: 215. In some embodiments, the TRBC guide RNA comprises a guide sequence comprising a sequence of any one of SEQ ID NOs: 201-265. In some embodiments, the TRBC guide RNA comprises a guide sequence comprising a sequence of SEQ ID NO: 215.

In some embodiments, the TRBC guide RNA comprises a sequence of any one of SEQ ID NOs: 1201-1265. In some embodiments, the TRBC guide RNA comprises a sequence of SEQ ID NO: 1215. In some embodiments, the TRBC guide RNA comprises a sequence of any one of SEQ ID NOs: 2201-2265 and 3005. In some embodiments, the TRBC guide RNA comprises a sequence of SEQ ID NO: 2215 or 3005. In some embodiments, the TRBC guide RNA comprises a sequence of SEQ ID NO: 2215. In some embodiments, the TRBC guide RNA comprises a sequence of SEQ ID NO: 3005.

In some embodiments, the TRBC guide RNA is a single guide RNA (sgRNA) comprising a sequence of any one of the sgRNA sequences listed in Table 3.

Additional embodiments of TRBC guide RNAs are provided herein, including e.g., exemplary modifications to the guide RNA.

### 1. Genetic Modifications to TRBC

In some embodiments, the methods and compositions disclosed herein genetically modify at least one nucleotide of the TRBC1 gene in a cell. In some embodiments, the genetic modification to TRBC1 reduces or eliminates the expression of TRBC1 protein on the surface of the genetically modified cell (or engineered cell). Genetic modifications encompass the population of modifications that results from contact with a genomic editing system (e.g., the population of edits that result from Cas9 and a TRBC1 guide RNA, or the population of edits that result from BC22 and a TRBC1 guide RNA).

In some embodiments, the methods and compositions disclosed herein genetically modify at least one nucleotide of the TRBC2 gene in a cell. In some embodiments, the genetic modification to TRBC2 reduces or eliminates the expression of TRBC2 protein on the surface of the genetically modified cell (or engineered cell). Genetic modifications encompass the population of modifications that results from contact with a genomic editing system (e.g., the population of edits that result from Cas9 and a TRBC2 guide RNA, or the population of edits that result from BC22 and a TRBC2 guide RNA).

In some embodiments, the genetic modification comprises at least one nucleotide within the genomic coordinates chosen from: (a) chr7:142791862-142793149 or (b) chr7:142801104-142802543. In some embodiments, the genetic modification comprises at least one nucleotide within the genomic coordinates chosen from any of the genomic coordinates listed in Table 3.

In some embodiments, the genetic modification comprises at least one nucleotide within the genomic coordinates chosen from: chr7:142792690-142792714 and chr7:142792693-142792717. In some embodiments, the genetic modification comprises at least 5 contiguous nucleotides within the genomic coordinates chosen from: chr7:142792690-142792714 and chr7:142792693-142792717. In some embodiments, the genetic modification comprises at least 10 contiguous nucleotides within the genomic coordinates chosen from: chr7:142792690-142792714 and chr7:142792693-142792717.

In some embodiments, the genetic modification comprises at least one nucleotide within the genomic coordinates chosen from: chr7:142791761-142791785; chr7:142791820-142791844; chr7:142791939-142791963; and chr7:142791756-142791780. In some embodiments, the genetic modification comprises at least 5 contiguous nucleotides within the genomic coordinates chosen from: chr7:142791761-142791785; chr7:142791820-142791844; chr7:142791939-142791963; and chr7:142791756-142791780. In some embodiments, the genetic modification comprises at least 10 contiguous nucleotides within the genomic coordinates chosen from: chr7:142791761-142791785; chr7:142791820-142791844; chr7:142791939-142791963; and chr7:142791756-142791780.

In some embodiments, the genetic modification comprises at least one nucleotide within the genomic coordinates chosen from: chr7:142801104-142801124; chr7:142802103-142802127; and chr7:142802106-142802130. In some embodiments, the genetic modification comprises at least 5 contiguous nucleotides within the genomic coordinates chosen from: chr7:142801104-142801124; chr7:142802103-142802127; and chr7:142802106-142802130. In some embodiments, the genetic modification comprises at least 10 contiguous nucleotides within the genomic coordinates chosen from chr7:142801104-142801124; chr7:142802103-142802127; and chr7:142802106-142802130.

In some embodiments, the genetic modification comprises at least one nucleotide within the genomic coordinates chosen from: chr7:142792690-142792714; chr7:142802103-142802127; and chr7:142802106-14280213. In some embodiments, the genetic modification comprises at least 5 contiguous nucleotides within the genomic coordinates chosen from: chr7:142792690-142792714; chr7:142802103-142802127; and chr7:142802106-14280213. In some embodiments, the genetic modification comprises at least 10 contiguous nucleotides within the genomic coordinates chosen from chr7:142792690-142792714; chr7:142802103-142802127; and chr7:142802106-14280213.

In some embodiments, the genetic modification comprises at least one indel, at least one C to T substitution, or at least one A to G substitution within the genomic coordinates chosen from: (a) chr7:142792690-142792714 and chr7:142792693-142792717; (b) chr7:142791761-142791785; chr7:142791820-142791844; chr7:142791939-142791963; and chr7:142791756-142791780; or (c) chr7:142801104-142801124; chr7:142802103-142802127; and chr7:142802106-142802130.

In some embodiments, the genetic modification comprises at least one indel, at least one C to T substitution, or at least one A to G substitution within the genomic coordinates chosen from: chr7:142792690-142792714; chr7:142802103-142802127; and chr7:142802106-14280213.

In some embodiments, the modification to TRBC comprises any one or more of an insertion, deletion, substitution, or deamination of at least one nucleotide in a target sequence. In some embodiments, the modification to TRBC comprises an insertion of 1, 2, 3, 4 or 5 or more nucleotides in a target sequence. In some embodiments, the modification to TRBC comprises a deletion of 1, 2, 3, 4 or 5 or more nucleotides in a target sequence. In other embodiments, the modification to TRBC comprises an insertion of 1, 2, 3, 4, 5, 6, 7, 8, 9, 10, 15, 20 or 25 or more nucleotides in a target sequence. In other embodiments, the modification to TRBC comprises a deletion of 1, 2, 3, 4, 5, 6, 7, 8, 9, 10, 15, 20 or 25 or more nucleotides in a target sequence. In some embodiments, the modification to TRBC comprises an indel, which is generally defined in the art as an insertion or deletion of less than 1000 base pairs (bp). In some embodiments, the modification to TRBC comprises an indel which results in a frameshift mutation in a target sequence. In some embodiments, the modification to TRBC comprises a substitution of 1, 2, 3, 4, 5, 6, 7, 8, 9, 10, 15, 20 or 25 or more nucleotides in a target sequence. In some embodiments, the modification to TRBC comprises one or more of an insertion, deletion, or substitution of nucleotides resulting from the incorporation of a template nucleic acid. In some embodiments, the modification to TRBC is not transient.

In some embodiments, the methods and compositions disclosed herein modify the TRBC gene in a cell using an RNA-guided DNA binding agent (e.g., a Cas enzyme). In some embodiments, the RNA-guided DNA binding agent is Cas9. In some embodiments, the RNA-guided DNA binding agent cuts within the TRBC1 gene, wherein the TRBC1 guide RNA targets a TRBC1 genomic target sequence comprising at least 10 contiguous nucleotides chr7:142791862-142793149. In some embodiments, the RNA-guided DNA binding agent cuts within the TRBC2 gene, wherein the TRBC3 guide RNA targets a TRBC2 genomic target sequence comprising at least 10 contiguous nucleotides within chr7:142801104-142802543.

In some embodiments, the genetic modification to TRBC results in utilization of an out-of-frame stop codon. In some embodiments, the genetic modification to TRBC results in exon skipping during splicing. In some embodiments, the genetic modification to TRBC1 results in reduced or eliminated TRBC1 protein expression by the cell. In some embodiments, the modification to the TRBC1 gene results in reduced or eliminated TRBC1 protein expression on the surface of the cell. In some embodiments, the genetic modification to TRBC2 results in reduced or eliminated TRBC2 protein expression by the cell. In some embodiments, the modification to the TRBC2 gene results in eliminated TRBC2 protein expression on the surface of the cell.

In some embodiments, TRBC1 expression on the surface of a cell is reduced as a result of the genetic modification to TRBC1. In some embodiments, TRBC1 expression on the surface of a cell is absent as a result of the genetic modification to TRBC1.

In some embodiments, TRBC2 expression on the surface of a cell is reduced as a result of the genetic modification to TRBC2. In some embodiments, TRBC2 expression on the surface of a cell is absent as a result of the genetic modification to TRBC2.

### 2. Efficacy of TRBC Guide RNAs

In some embodiments, the efficacy of a TRBC gRNA is determined when delivered or expressed together with other components forming an RNP. In some embodiments, the TRBC gRNA is expressed together with an RNA-guided DNA binding agent, such as a Cas protein, e.g. Cas9. In some embodiments, the TRBC gRNA is delivered to or expressed in a cell line that already stably expresses an RNA-guided DNA nuclease, such as a Cas nuclease or nickase, e.g. Cas9 nuclease or nickase. In some embodiments the TRBC gRNA is delivered to a cell as part of an RNP. In some embodiments, the TRBC gRNA is delivered to a cell along with a mRNA encoding an RNA-guided DNA nuclease, such as a Cas nuclease or nickase, e.g. Cas9 nuclease or nickase.

As described herein, use of an RNA-guided DNA nuclease and a TRBC guide RNA disclosed herein can lead to double-stranded breaks in the DNA which can produce errors in the form of insertion/deletion (indel) mutations upon repair by cellular machinery. Many mutations due to indels alter the reading frame or introduce premature stop codons and, therefore, produce a non-functional protein.

In some embodiments, the efficacy of particular TRBC gRNAs is determined based on in vitro models. In some embodiments, the in vitro model is HEK293 cells stably expressing Cas9 (HEK293_Cas9). In some embodiments the in vitro model is a peripheral blood mononuclear cell (PBMC). In some embodiments, the in vitro model is a T cell, such as primary human T cells. With respect to using primary cells, commercially available primary cells can be used to provide greater consistency between experiments. In some embodiments, the number of off-target sites at which a deletion or insertion occurs in an in vitro model (e.g., in T cell) is determined, e.g., by analyzing genomic DNA from transfected cells in vitro with Cas9 mRNA and the guide RNA. In some embodiments, such a determination comprises analyzing genomic DNA from the cells transfected in vitro with Cas9 mRNA, the TRBC guide RNA, and a donor oligonucleotide. Exemplary procedures for such determinations are provided in the working examples in which HEK293 cells, PBMCs, and human CD3+ T cells are used.

In some embodiments, the efficacy of particular TRBC gRNAs is determined across multiple in vitro cell models for a gRNA selection process. In some embodiments, a cell line comparison of data with selected TRBC gRNAs is performed. In some embodiments, cross screening in multiple cell models is performed.

In some embodiments, the efficacy of a TRBC guide RNA is measured by percent indels of TRBC. In some embodiments, the percent editing of TRBC is compared to the percent indels necessary to achieve knockdown of the TRBC protein products

In some embodiments, the efficacy of a guide RNA is measured by reduced or eliminated expression of a component of the T-cell receptor (TCR). In embodiments, the reduced or eliminated expression of a component of the T-cell receptor (TCR) includes reduced or eliminated expression of TRBC. In some embodiments, said reduced or eliminated expression of said component of the TCR is the result of introduction of one or more, e.g., one or two, e.g., one TRBC gRNA molecule described herein to said component of the TCR into said cell. In embodiments, said reduced or eliminated expression of a component of the TCR is as measured by flow cytometry, e.g., as described herein.

In some embodiments, the efficacy of a TRBC guide RNA is measured by the number and/or frequency of indels at off-target sequences within the genome of the target cell type, such as a T cell. In some embodiments, efficacious guide RNAs are provided which produce indels at off target sites at very low frequencies (e.g., <5%) in a cell population and/or relative to the frequency of indel creation at the target site. Thus, the disclosure provides for guide RNAs which do not exhibit off-target indel formation in the target cell type (e.g., a T cell), or which produce a frequency of off-target indel formation of <5% in a cell population and/or relative to the frequency of indel creation at the target site. In some embodiments, the disclosure provides guide RNAs which do not exhibit any off target indel formation in the target cell type (e.g., T cell). In some embodiments, guide RNAs are provided which produce indels at less than 5 off-target sites, e.g., as evaluated by one or more methods described herein. In some embodiments, guide RNAs are provided which produce indels at less than or equal to 4, 3, 2, or 1 off-target site(s) e.g., as evaluated by one or more methods described herein. In some embodiments, the off-target site(s) does not occur in a protein coding region in the target cell (e.g., hepatocyte) genome.

In some embodiments, detecting gene editing events, such as the formation of insertion/deletion (“indel”) mutations and homology directed repair (HDR) events in target DNA utilize linear amplification with a tagged primer and isolating the tagged amplification products (herein after referred to as “LAM-PCR,” or “Linear Amplification (LA)” method).

In some embodiments, the efficacy of a guide RNA is measured by the levels of functional protein complexes comprising the expressed protein product of the gene. In some embodiments, the efficacy of a guide RNA is measured by flow cytometric analysis of TCR expression by which the live population of edited cells is analyzed for loss of the TCR.

### F. CIITA Guide RNAs

The methods and compositions provided herein disclose CIITA guide RNAs useful for reducing the expression of MHC class II protein on the surface of a cell. In some embodiments, such guide RNAs direct an RNA-guided DNA binding agent to a CIITA genomic target sequence and may be referred to herein as “CIITA guide RNAs.” In some embodiments, the CIITA guide RNA directs an RNA-guided DNA binding agent to a human CIITA genomic target sequence. In some embodiments, the CIITA guide RNA comprises a guide sequence selected from SEQ ID NOs: 301, 302, 304-576.

In some embodiments, the methods and compositions disclosed herein comprise a CIITA guide RNA comprising a guide sequence that targets a CIITA genomic target sequence comprising at least 10 contiguous nucleotides within the genomic coordinates chosen from: (a) chr16:10877363-10907788 or (b) chr16:10906515-10908136. In some embodiments, the methods and compositions disclosed herein comprise a CIITA guide RNA comprising a guide sequence that targets a CIITA genomic target sequence comprising at least one nucleotide within the genomic coordinates chosen from: (a) chr16:10877363-10907788 or (b) chr16:10906515-10908136.

In some embodiments, the methods and compositions disclosed herein comprise a CIITA guide RNA comprising a guide sequence that directs an RNA-guided DNA binding agent to make cut in a CIITA gene, wherein the CIITA guide RNA targets a CIITA genomic target sequence comprising at least 10 contiguous nucleotides within the genomic coordinates chosen from: (a) chr16:10877363-10907788 or (b) chr16:10906515-10908136. In some embodiments, the methods and compositions disclosed herein comprise a CIITA guide RNA comprising a guide sequence that directs an RNA-guided DNA binding agent to make cut in a CIITA gene, wherein the CIITA guide RNA targets a CIITA genomic target sequence comprising at least one nucleotide within the genomic coordinates chosen from: (a) chr16:10877363-10907788 or (b) chr16:10906515-10908136.

In some embodiments, the methods and compositions disclose a CIITA guide RNA that directs an RNA-guided DNA binding agent to induce a double stranded break (DSB) or a single-stranded break (SSB) in a CIITA genomic target sequence. In some embodiments, the methods and compositions disclose a CIITA guide RNA that directs an RNA-guided DNA binding agent to make a cut in a CIITA genomic target sequence. In embodiments wherein the RNA-guided DNA cutting agent is Cas9, the cut or “cut site” occurs at the third base from the protospacer adjacent motif (PAM) sequence.

In some embodiments, a composition is provided comprising a CIITA guide RNA described herein and an RNA-guided DNA binding agent or a nucleic acid encoding an RNA-guided DNA binding agent.

In some embodiments, a composition is provided comprising a CIITA single-guide RNA (sgRNA) comprising a guide sequence that targets a genomic target comprising at least 10 contiguous nucleotides within the genomic coordinates chosen from: (a) chr16:10877363-10907788 or (b) chr16:10906515-10908136. In some embodiments, a composition is provided comprising a CIITA sgRNA described herein and an RNA-guided DNA binding agent or a nucleic acid encoding an RNA-guided DNA binding agent.

In some embodiments, a composition is provided comprising a CIITA dual-guide RNA (dgRNA) comprising a guide sequence that targets a genomic target comprising at least 10 contiguous nucleotides within the genomic coordinates chosen from: (a) chr16:10877363-10907788 or (b) chr16:10906515-10908136. In some embodiments, a composition is provided comprising a CIITA dgRNA described herein and an RNA-guided DNA binding agent or a nucleic acid encoding an RNA-guided DNA binding agent.

Exemplary CIITA guide sequences are shown below in Table 4.

TABLE 4
Exemplary CIITA guide sequences.

| | | | Exemplary | Exemplary Guide | | |
|---|---|---|---|---|---|---|
| | | | Guide RNA | RNA Modified | | |
| | SEQ ID | | Full Sequence | Sequence (SEQ ID | | |
| | NO to | | (SEQ ID NOS: | Nos: 2301, 2302, | | Genomic |
| Guide | the Guide | Guide | 1301, 1302, | 2304-2576 and | SEQ ID | Coordinates |
| ID | Sequence | Sequence | 1304-1576) | 3016-3205) | NOS | (hg38) |
| G026584 | 301 | UCAAAGUA | UCAAAGUACCCUA | mU*mC*mA*mAmAGUm | 2301 | chr16: |
| | | CCCUACAG | CAGGAGGACCAGU | AmCCmCUmACAGGmAG | | 10907504- |
| | | GAGGACCA | UGUAGCUCCCUGA | GAmCCAmGUUGmUmAm | | 10907528 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G026585 | 302 | AGUACCCU | AGUACCCUACAGG | mA*mG*mU*mAmCCCm | 2302 | chr16: |
| | | ACAGGAGG | AGGACCAGUUCGU | UmACmAGmGAGGAmCC | | 10907508- |
| | | ACCAGUUC | UGUAGCUCCCUGA | AGmUUCmGUUGmUmAm | | 10907532 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029013 | 304 | UACCUGUC | UACCUGUCAGAGC | mU*mA*mC*mCmUGUm | 2304 | chr16: |
| | | AGAGCCCC | CCCAAGGUAAAGU | CmAGmAGmCCCCAmAG | | 10877363- |
| | | AAGGUAAA | UGUAGCUCCCUGA | GUmAAAmGUUGmUmAm | | 10877387 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029014 | 305 | UUUACCUU | UUUACCUUGGGGC | mU*mU*mU*mAmCCUm | 2305 | chr16: |
| | | GGGGCUCU | UCUGACAGGUAGU | UmGGmGGmCUCUGmAC | | 10877363- |
| | | GACAGGUA | UGUAGCUCCCUGA | AGmGUAmGUUGmUmAm | | 10877387 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029015 | 306 | UUUUACCU | UUUUACCUUGGGG | mU*mU*mU*mUmACCm | 2306 | chr16: |
| | | UGGGGCUC | CUCUGACAGGUGU | UmUGmGGmGCUCUmGA | | 10877364- |
| | | UGACAGGU | UGUAGCUCCCUGA | CAmGGUmGUUGmUmAm | | 10877388 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029016 | 307 | GCCUGGGA | GCCUGGGAGGGAA | mG*mC*mC*mUmGGGm | 2307 | chr16: |
| | | GGGAAGAC | GACAAUGCUCAGU | AmGGmGAmAGACAmAU | | 10895259- |
| | | AAUGCUCA | UGUAGCUCCCUGA | GCmUCAmGUUGmUmAm | | 10895283 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029017 | 308 | UCUUCCCU | UCUUCCCUCCCAG | mU*mC*mU*mUmCCCm | 2308 | chr16: |
| | | CCCAGGCA | GCAGCUCACAGGU | UmCCmCAmGGCAGmCU | | 10895268- |
| | | GCUCACAG | UGUAGCUCCCUGA | CAmCAGmGUUGmUmAm | | 10895292 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029018 | 309 | UCCCUCCC | UCCCUCCCAGGCA | mU*mC*mC*mCmUCCm | 2309 | chr16: |
| | | AGGCAGCU | GCUCACAGUGUGU | CmAGmGCmAGCUCmAC | | 10895271- |
| | | CACAGUGU | UGUAGCUCCCUGA | AGmUGUmGUUGmUmAm | | 10895295 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029019 | 310 | CUCACAGU | CUCACAGUGUGCC | mC*mU*mC*mAmCAGm | 2310 | chr16: |
| | | GUGCCACC | ACCAUGGAGUUGU | UmGUmGCmCACCAmUG | | 10895285- |
| | | AUGGAGUU | UGUAGCUCCCUGA | GAmGUUmGUUGmUmAm | | 10895309 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029020 | 311 | UCACAGUG | UCACAGUGUGCCA | mU*mC*mA*mCmAGUm | 2311 | chr16: |
| | | UGCCACCA | CCAUGGAGUUGGU | GmUGmCCmACCAUmGG | | 10895286- |
| | | UGGAGUUG | UGUAGCUCCCUGA | AGmUUGmGUUGmUmAm | | 10895310 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029021 | 312 | CACAGUGU | CACAGUGUGCCAC | mC*mA*mC*mAmGUGm | 2312 | chr16: |
| | | GCCACCAU | CAUGGAGUUGGGU | UmGCmCAmCCAUGmGA | | 10895287- |
| | | GGAGUUGG | UGUAGCUCCCUGA | GUmUGGmGUUGmUmAm | | 10895311 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029022 | 313 | GAAGAGAU | GAAGAGAUUGAGC | mG*mA*mA*mGmAGAm | 2313 | chr16: |
| | | UGAGCUCU | UCUACUCAGGUGU | UmUGmAGmCUCUAmCU | | 10895406- |
| | | ACUCAGGU | UGUAGCUCCCUGA | CAmGGUmGUUGmUmAm | | 10895430 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029023 | 314 | ACCUGAGU | ACCUGAGUAGAGC | mA*mC*mC*mUmGAGm | 2314 | chr16: |
| | | AGAGCUCA | UCAAUCUCUUCGU | UmAGmAGmCUCAAmUC | | 10895406- |
| | | AUCUCUUC | UGUAGCUCCCUGA | UCmUUCmGUUGmUmAm | | 10895430 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029024 | 315 | GAGAUUGA | GAGAUUGAGCUCU | mG*mA*mG*mAmUUGm | 2315 | chr16: |
| | | GCUCUACU | ACUCAGGUGGGGU | AmGCmUCmUACUCmAG | | 10895409- |
| | | CAGGUGGG | UGUAGCUCCCUGA | GUmGGGmGUUGmUmAm | | 10895433 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029025 | 316 | AUUGAGCU | AUUGAGCUCUACU | mA*mU*mU*mGmAGCm | 2316 | chr16: |
| | | CUACUCAG | CAGGUGGGCCCGU | UmCUmACmUCAGGmUG | | 10895412- |
| | | GUGGGCCC | UGUAGCUCCCUGA | GGmCCCmGUUGmUmAm | | 10895436 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029026 | 317 | UUGAGCUC | UUGAGCUCUACUC | mU*mU*mG*mAmGCUm | 2317 | chr16: |
| | | UACUCAGG | AGGUGGGCCCUGU | CmUAmCUmCAGGUmGG | | 10895413- |
| | | UGGGCCCU | UGUAGCUCCCUGA | GCmCCUmGUUGmUmAm | | 10895437 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029027 | 318 | AGGUGGGC | AGGUGGGCCCUCC | mA*mG*mG*mUmGGGm | 2318 | chr16: |
| | | CCUCCUCC | UCCCUCUGGUCGU | CmCCmUCmCUCCCmUC | | 10895426- |
| | | CUCUGGUC | UGUAGCUCCCUGA | UGmGUCmGUUGmUmAm | | 10895450 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029028 | 319 | CUUUUCCU | CUUUUCCUCCCAG | mC*mU*mU*mUmUCCm | 2319 | chr16: |
| | | CCCAGAAC | AACCCGACACAGU | UmCCmCAmGAACCmCG | | 10895655- |
| | | CCGACACA | UGUAGCUCCCUGA | ACmACAmGUUGmUmAm | | 10895679 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029029 | 320 | GUCUGUGU | GUCUGUGUCGGGU | mG*mU*mC*mUmGUGm | 2320 | chr16: |
| | | CGGGUUCU | UCUGGGAGGAAGU | UmCGmGGmUUCUGmGG | | 10895658- |
| | | GGGAGGAA | UGUAGCUCCCUGA | AGmGAAmGUUGmUmAm | | 10895682 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029030 | 321 | AACCCGAC | AACCCGACACAGA | mA*mA*mC*mCmCGAm | 2321 | chr16: |
| | | ACAGACAC | CACCAUCAACUGU | CmACmAGmACACCmAU | | 10895668- |
| | | CAUCAACU | UGUAGCUCCCUGA | CAmACUmGUUGmUmAm | | 10895692 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029031 | 322 | GGCUUAUG | GGCUUAUGCCAAU | mG*mG*mC*mUmUAUm | 2322 | chr16: |
| | | CCAAUAUC | AUCGGUGAGGAGU | GmCCmAAmUAUCGmGU | | 10895747- |
| | | GGUGAGGA | UGUAGCUCCCUGA | GAmGGAmGUUGmUmAm | | 10895771 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029032 | 323 | CUUCCUCA | CUUCCUCACCGAU | mC*mU*mU*mCmCUCm | 2323 | chr16: |
| | | CCGAUAUU | AUUGGCAUAAGGU | AmCCmGAmUAUUGmGC | | 10895749- |
| | | GGCAUAAG | UGUAGCUCCCUGA | AUmAAGmGUUGmUmAm | | 10895773 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029033 | 324 | GCUUCCUC | GCUUCCUCACCGA | mG*mC*mU*mUmCCUm | 2324 | chr16: |
| | | ACCGAUAU | UAUUGGCAUAAGU | CmACmCGmAUAUUmGG | | 10895750- |
| | | UGGCAUAA | UGUAGCUCCCUGA | CAmUAAmGUUGmUmAm | | 10895774 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029034 | 325 | UGCCAAUA | UGCCAAUAUCGGU | mU*mG*mC*mCmAAUm | 2325 | chr16: |
| | | UCGGUGAG | GAGGAAGCACCGU | AmUCmGGmUGAGGmAA | | 10895753- |
| | | GAAGCACC | UGUAGCUCCCUGA | GCmACCmGUUGmUmAm | | 10895777 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029035 | 326 | GGUGCUUC | GGUGCUUCCUCAC | mG*mG*mU*mGmCUUm | 2326 | chr16: |
| | | CUCACCGA | CGAUAUUGGCAGU | CmCUmCAmCCGAUmAU | | 10895753- |
| | | UAUUGGCA | UGUAGCUCCCUGA | UGmGCAmGUUGmUmAm | | 10895777 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029036 | 327 | GCCAAUAU | GCCAAUAUCGGUG | mG*mC*mC*mAmAUAm | 2327 | chr16: |
| | | CGGUGAGG | AGGAAGCACCUGU | UmCGmGUmGAGGAmAG | | 10895754- |
| | | AAGCACCU | UGUAGCUCCCUGA | CAmCCUmGUUGmUmAm | | 10895778 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029037 | 328 | UUUUCCUU | UUUUCCUUGUCUG | mU*mU*mU*mUmCCUm | 2328 | chr16: |
| | | GUCUGGGC | GGCAGCGGAACGU | UmGUmCUmGGGCAmGC | | 10898651- |
| | | AGCGGAAC | UGUAGCUCCCUGA | GGmAACmGUUGmUmAm | | 10898675 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029038 | 329 | GGGCAGCG | GGGCAGCGGAACU | mG*mG*mG*mCmAGCm | 2329 | chr16: |
| | | GAACUGGA | GGACCAGUAUGGU | GmGAmACmUGGACmCA | | 10898663- |
| | | CCAGUAUG | UGUAGCUCCCUGA | GUmAUGmGUUGmUmAm | | 10898687 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029039 | 330 | GAACUGGA | GAACUGGACCAGU | mG*mA*mA*mCmUGGm | 2330 | chr16: |
| | | CCAGUAUG | AUGUCUUCCAGGU | AmCCmAGmUAUGUmCU | | 10898671- |
| | | UCUUCCAG | UGUAGCUCCCUGA | UCmCAGmGUUGmUmAm | | 10898695 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029040 | 331 | AACUGGAC | AACUGGACCAGUA | mA*mA*mC*mUmGGAm | 2331 | chr16: |
| | | CAGUAUGU | UGUCUUCCAGGGU | CmCAmGUmAUGUCmUU | | 10898672- |
| | | CUUCCAGG | UGUAGCUCCCUGA | CCmAGGmGUUGmUmAm | | 10898696 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029041 | 332 | AUGUCUUC | AUGUCUUCCAGGA | mA*mU*mG*mUmCUUm | 2332 | chr16: |
| | | CAGGACUC | CUCCCAGCUGGGU | CmCAmGGmACUCCmCA | | 10898684- |
| | | CCAGCUGG | UGUAGCUCCCUGA | GCmUGGmGUUGmUmAm | | 10898708 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029042 | 333 | UUACUGAA | UUACUGAAAAUGU | mU*mU*mA*mCmUGAm | 2333 | chr16: |
| | | AAUGUCCU | CCUUGCUCAGGGU | AmAAmUGmUCCUUmGC | | 10898712- |
| | | UGCUCAGG | UGUAGCUCCCUGA | UCmAGGmGUUGmUmAm | | 10898736 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029043 | 334 | AACUUACU | AACUUACUGAAAA | mA*mA*mC*mUmUACm | 2334 | chr16: |
| | | GAAAAUGU | UGUCCUUGCUCGU | UmGAmAAmAUGUCmCU | | 10898715- |
| | | CCUUGCUC | UGUAGCUCCCUGA | UGmCUCmGUUGmUmAm | | 10898739 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029044 | 335 | AAACUUAC | AAACUUACUGAAA | mA*mA*mA*mCmUUAm | 2335 | chr16: |
| | | UGAAAAUG | AUGUCCUUGCUGU | CmUGmAAmAAUGUmCC | | 10898716- |
| | | UCCUUGCU | UGUAGCUCCCUGA | UUmGCUmGUUGmUmAm | | 10898740 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029045 | 336 | CCACCCAC | CCACCCACCACAA | mC*mC*mA*mCmCCAm | 2336 | chr16: |
| | | CACAAACU | ACUUACUGAAAGU | CmCAmCAmAACUUmAC | | 10898727- |
| | | UACUGAAA | UGUAGCUCCCUGA | UGmAAAmGUUGmUmAm | | 10898751 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029046 | 337 | UGUGCUCU | UGUGCUCUACUUU | mU*mG*mU*mGmCUCm | 2337 | chr16: |
| | | ACUUUGAG | GAGAAAAACCAGU | UmACmUUmUGAGAmAA | | 10898906- |
| | | AAAAACCA | UGUAGCUCCCUGA | AAmCCAmGUUGmUmAm | | 10898930 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029047 | 338 | GGUUUUUC | GGUUUUUCUCAAA | mG*mG*mU*mUmUUUm | 2338 | chr16: |
| | | UCAAAGUA | GUAGAGCACAUGU | CmUCmAAmAGUAGmAG | | 10898907- |
| | | GAGCACAU | UGUAGCUCCCUGA | CAmCAUmGUUGmUmAm | | 10898931 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029048 | 339 | GGUCCUAU | GGUCCUAUGUGCU | mG*mG*mU*mCmCUAm | 2339 | chr16: |
| | | GUGCUCUA | CUACUUUGAGAGU | UmGUmGCmUCUACmUU | | 10898913- |
| | | CUUUGAGA | UGUAGCUCCCUGA | UGmAGAmGUUGmUmAm | | 10898937 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029049 | 340 | CAGAAAAG | CAGAAAAGUCAGA | mC*mA*mG*mAmAAAm | 2340 | chr16: |
| | | UCAGAAAA | AAAGACGUGAGGU | GmUCmAGmAAAAGmAC | | 10898983- |
| | | GACGUGAG | UGUAGCUCCCUGA | GUmGAGmGUUGmUmAm | | 10899007 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029050 | 341 | AGAAAAGU | AGAAAAGUCAGAA | mA*mG*mA*mAmAAGm | 2341 | chr16: |
| | | CAGAAAAG | AAGACGUGAGUGU | UmCAmGAmAAAGAmCG | | 10898984- |
| | | ACGUGAGU | UGUAGCUCCCUGA | UGmAGUmGUUGmUmAm | | 10899008 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029051 | 342 | GAAAAGUC | GAAAAGUCAGAAA | mG*mA*mA*mAmAGUm | 2342 | chr16: |
| | | AGAAAAGA | AGACGUGAGUGGU | CmAGmAAmAAGACmGU | | 10898985- |
| | | CGUGAGUG | UGUAGCUCCCUGA | GAmGUGmGUUGmUmAm | | 10899009 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029052 | 343 | UCACUCAC | UCACUCACGUCUU | mU*mC*mA*mCmUCAm | 2343 | chr16: |
| | | GUCUUUUC | UUCUGACUUUUGU | CmGUmCUmUUUCUmGA | | 10898986- |
| | | UGACUUUU | UGUAGCUCCCUGA | CUmUUUmGUUGmUmAm | | 10899010 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029053 | 344 | CUCACUCA | CUCACUCACGUCU | mC*mU*mC*mAmCUCm | 2344 | chr16: |
| | | CGUCUUUU | UUUCUGACUUUGU | AmCGmUCmUUUUCmUG | | 10898987- |
| | | CUGACUUU | UGUAGCUCCCUGA | ACmUUUmGUUGmUmAm | | 10899011 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029054 | 345 | AAGUCAGA | AAGUCAGAAAAGA | mA*mA*mG*mUmCAGm | 2345 | chr16: |
| | | AAAGACGU | CGUGAGUGAGCGU | AmAAmAGmACGUGmAG | | 10898988- |
| | | GAGUGAGC | UGUAGCUCCCUGA | UGmAGCmGUUGmUmAm | | 10899012 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029055 | 346 | AGUCAGAA | AGUCAGAAAAGAC | mA*mG*mU*mCmAGAm | 2346 | chr16: |
| | | AAGACGUG | GUGAGUGAGCCGU | AmAAmGAmCGUGAmGU | | 10898989- |
| | | AGUGAGCC | UGUAGCUCCCUGA | GAmGCCmGUUGmUmAm | | 10899013 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029056 | 347 | AAAAGACG | AAAAGACGUGAGU | mA*mA*mA*mAmGACm | 2347 | chr16: |
| | | UGAGUGAG | GAGCCCCUCCCGU | GmUGmAGmUGAGCmCC | | 10898995- |
| | | CCCCUCCC | UGUAGCUCCCUGA | CUmCCCmGUUGmUmAm | | 10899019 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029057 | 348 | GACGUGAG | GACGUGAGUGAGC | mG*mA*mC*mGmUGAm | 2348 | chr16: |
| | | UGAGCCCC | CCCUCCCUGAUGU | GmUGmAGmCCCCUmCC | | 10898999- |
| | | UCCCUGAU | UGUAGCUCCCUGA | CUmGAUmGUUGmUmAm | | 10899023 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029058 | 349 | CUGCAGAG | CUGCAGAGAAAAC | mC*mU*mG*mCmAGAm | 2349 | chr16: |
| | | AAAACAUG | AUGUGAUCAGCGU | GmAAmAAmCAUGUmGA | | 10901489- |
| | | UGAUCAGC | UGUAGCUCCCUGA | UCmAGCmGUUGmUmAm | | 10901513 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029059 | 350 | GCUGCAGA | GCUGCAGAGAAAA | mG*mC*mU*mGmCAGm | 2350 | chr16: |
| | | GAAAACAU | CAUGUGAUCAGGU | AmGAmAAmACAUGmUG | | 10901490- |
| | | GUGAUCAG | UGUAGCUCCCUGA | AUmCAGmGUUGmUmAm | | 10901514 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029060 | 351 | GGCUGCAG | GGCUGCAGAGAAA | mG*mG*mC*mUmGCAm | 2351 | chr16: |
| | | AGAAAACA | ACAUGUGAUCAGU | GmAGmAAmAACAUmGU | | 10901491- |
| | | UGUGAUCA | UGUAGCUCCCUGA | GAmUCAmGUUGmUmAm | | 10901515 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029061 | 352 | UUUCUCUG | UUUCUCUGCAGCC | mU*mU*mU*mCmUCUm | 2352 | chr16: |
| | | CAGCCUUC | UUCCCAGAGGAGU | GmCAmGCmCUUCCmCA | | 10901502- |
| | | CCAGAGGA | UGUAGCUCCCUGA | GAmGGAmGUUGmUmAm | | 10901526 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029062 | 353 | CAGCCUUC | CAGCCUUCCCAGA | mC*mA*mG*mCmCUUm | 2353 | chr16: |
| | | CCAGAGGA | GGAGCUUCCGGGU | CmCCmAGmAGGAGmCU | | 10901510- |
| | | GCUUCCGG | UGUAGCUCCCUGA | UCmCGGmGUUGmUmAm | | 10901534 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029063 | 354 | UUCCAGUG | UUCCAGUGCUUCA | mU*mU*mC*mCmAGUm | 2354 | chr16: |
| | | CUUCAGGU | GGUCUGCCGGAGU | GmCUmUCmAGGUCmUG | | 10901529- |
| | | CUGCCGGA | UGUAGCUCCCUGA | CCmGGAmGUUGmUmAm | | 10901553 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029064 | 355 | ACACCUGG | ACACCUGGCUUCC | mA*mC*mA*mCmCUGm | 2355 | chr16: |
| | | CUUCCAGU | AGUGCUUCAGGGU | GmCUmUCmCAGUGmCU | | 10901538- |
| | | GCUUCAGG | UGUAGCUCCCUGA | UCmAGGmGUUGmUmAm | | 10901562 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029065 | 356 | CAGCCCAC | CAGCCCACCUGCC | mC*mA*mG*mCmCCAm | 2356 | chr16: |
| | | CUGCCCUG | CUGCACACCUGGU | CmCUmGCmCCUGCmAC | | 10901555- |
| | | CACACCUG | UGUAGCUCCCUGA | ACmCUGmGUUGmUmAm | | 10901579 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029066 | 357 | CACAGCCC | CACAGCCCACUUC | mC*mA*mC*mAmGCCm | 2357 | chr16: |
| | | ACUUCCUC | CUCACAGCUGAGU | CmACmUUmCCUCAmCA | | 10902017- |
| | | ACAGCUGA | UGUAGCUCCCUGA | GCmUGAmGUUGmUmAm | | 10902041 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029067 | 358 | ACAGCCCA | ACAGCCCACUUCC | mA*mC*mA*mGmCCCm | 2358 | chr16: |
| | | CUUCCUCA | UCACAGCUGAGGU | AmCUmUCmCUCACmAG | | 10902018- |
| | | CAGCUGAG | UGUAGCUCCCUGA | CUmGAGmGUUGmUmAm | | 10902042 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029068 | 359 | CUGCCUGC | CUGCCUGCGCUGU | mC*mU*mG*mCmCUGm | 2359 | chr16: |
| | | GCUGUUCA | UCAACCAGGAGGU | CmGCmUGmUUCAAmCC | | 10902114- |
| | | ACCAGGAG | UGUAGCUCCCUGA | AGmGAGmGUUGmUmAm | | 10902138 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029069 | 360 | CCUGCGCU | CCUGCGCUGUUCA | mC*mC*mU*mGmCGCm | 2360 | chr16: |
| | | GUUCAACC | ACCAGGAGCCAGU | UmGUmUCmAACCAmGG | | 10902117- |
| | | AGGAGCCA | UGUAGCUCCCUGA | AGmCCAmGUUGmUmAm | | 10902141 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029070 | 361 | CGCUGUUC | CGCUGUUCAACCA | mC*mG*mC*mUmGUUm | 2361 | chr16: |
| | | AACCAGGA | GGAGCCAGCCUGU | CmAAmCCmAGGAGmCC | | 10902121- |
| | | GCCAGCCU | UGUAGCUCCCUGA | AGmCCUmGUUGmUmAm | | 10902145 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029071 | 362 | ACCAGGAG | ACCAGGAGCCAGC | mA*mC*mC*mAmGGAm | 2362 | chr16: |
| | | CCAGCCUC | CUCCGGCCAGAGU | GmCCmAGmCCUCCmGG | | 10902130- |
| | | CGGCCAGA | UGUAGCUCCCUGA | CCmAGAmGUUGmUmAm | | 10902154 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029072 | 363 | GCCUCCGG | GCCUCCGGCCAGA | mG*mC*mC*mUmCCGm | 2363 | chr16: |
| | | CCAGAUGC | UGCGCCUGGAGGU | GmCCmAGmAUGCGmCC | | 10902141- |
| | | GCCUGGAG | UGUAGCUCCCUGA | UGmGAGmGUUGmUmAm | | 10902165 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029073 | 364 | CCGGCCAG | CCGGCCAGAUGCG | mC*mC*mG*mGmCCAm | 2364 | chr16: |
| | | AUGCGCCU | CCUGGAGAAAAGU | GmAUmGCmGCCUGmGA | | 10902145- |
| | | GGAGAAAA | UGUAGCUCCCUGA | GAmAAAmGUUGmUmAm | | 10902169 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029074 | 365 | AACAUACU | AACAUACUGGGAA | mA*mA*mC*mAmUACm | 2365 | chr16: |
| | | GGGAAUCU | UCUGGUCGGUUGU | UmGGmGAmAUCUGmGU | | 10902167- |
| | | GGUCGGUU | UGUAGCUCCCUGA | CGmGUUmGUUGmUmAm | | 10902191 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029075 | 366 | CUGCCUUU | CUGCCUUUGUCUC | mC*mU*mG*mCmCUUm | 2366 | chr16: |
| | | GUCUCUUG | UUGCAGUGCCUGU | UmGUmCUmCUUGCmAG | | 10902638- |
| | | CAGUGCCU | UGUAGCUCCCUGA | UGmCCUmGUUGmUmAm | | 10902662 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029076 | 367 | UUGUCUCU | UUGUCUCUUGCAG | mU*mU*mG*mUmCUCm | 2367 | chr16: |
| | | UGCAGUGC | UGCCUUUCUCCGU | UmUGmCAmGUGCCmUU | | 10902644- |
| | | CUUUCUCC | UGUAGCUCCCUGA | UCmUCCmGUUGmUmAm | | 10902668 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029077 | 368 | UGCCUUUC | UGCCUUUCUCCAG | mU*mG*mC*mCmUUUm | 2368 | chr16: |
| | | UCCAGUUC | UUCCUCGUUGAGU | CmUCmCAmGUUCCmUC | | 10902657- |
| | | CUCGUUGA | UGUAGCUCCCUGA | GUmUGAmGUUGmUmAm | | 10902681 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029078 | 369 | UCUCCCUG | UCUCCCUGAGGGA | mU*mC*mU*mCmCCUm | 2369 | chr16: |
| | | AGGGACCC | CCCAUCCAGUUGU | GmAGmGGmACCCAmUC | | 10902691- |
| | | AUCCAGUU | UGUAGCUCCCUGA | CAmGUUmGUUGmUmAm | | 10902715 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029079 | 370 | CUCCCUGA | CUCCCUGAGGGAC | mC*mU*mC*mCmCUGm | 2370 | chr16: |
| | | GGGACCCA | CCAUCCAGUUUGU | AmGGmGAmCCCAUmCC | | 10902692- |
| | | UCCAGUUU | UGUAGCUCCCUGA | AGmUUUmGUUGmUmAm | | 10902716 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029080 | 371 | CCUGAGGG | CCUGAGGGACCCA | mC*mC*mU*mGmAGGm | 2371 | chr16: |
| | | ACCCAUCC | UCCAGUUUGUCGU | GmACmCCmAUCCAmGU | | 10902695- |
| | | AGUUUGUC | UGUAGCUCCCUGA | UUmGUCmGUUGmUmAm | | 10902719 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029081 | 372 | GGACCCAU | GGACCCAUCCAGU | mG*mG*mA*mCmCCAm | 2372 | chr16: |
| | | CCAGUUUG | UUGUCCCCACCGU | UmCCmAGmUUUGUmCC | | 10902701- |
| | | UCCCCACC | UGUAGCUCCCUGA | CCmACCmGUUGmUmAm | | 10902725 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029082 | 373 | CCAGUUUG | CCAGUUUGUCCCC | mC*mC*mA*mGmUUUm | 2373 | chr16: |
| | | UCCCCACC | ACCAUCUCCACGU | GmUCmCCmCACCAmUC | | 10902709- |
| | | AUCUCCAC | UGUAGCUCCCUGA | UCmCACmGUUGmUmAm | | 10902733 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029083 | 374 | CAGUUUGU | CAGUUUGUCCCCA | mC*mA*mG*mUmUUGm | 2374 | chr16: |
| | | CCCCACCA | CCAUCUCCACUGU | UmCCmCCmACCAUmCU | | 10902710- |
| | | UCUCCACU | UGUAGCUCCCUGA | CCmACUmGUUGmUmAm | | 10902734 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029084 | 375 | AGUUUGUC | AGUUUGUCCCCAC | mA*mG*mU*mUmUGUm | 2375 | chr16: |
| | | CCCACCAU | CAUCUCCACUCGU | CmCCmCAmCCAUCmUC | | 10902711- |
| | | CUCCACUC | UGUAGCUCCCUGA | CAmCUCmGUUGmUmAm | | 10902735 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029085 | 376 | CAAAUCUC | CAAAUCUCUGAGG | mC*mA*mA*mAmUCUm | 2376 | chr16: |
| | | UGAGGCUG | CUGGAACAGGGGU | CmUGmAGmGCUGGmAA | | 10902752- |
| | | GAACAGGG | UGUAGCUCCCUGA | CAmGGGmGUUGmUmAm | | 10902776 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029086 | 377 | ACCAUGGU | ACCAUGGUAGAUG | mA*mC*mC*mAmUGGm | 2377 | chr16: |
| | | AGAUGAAU | AAUAUACUGGAGU | UmAGmAUmGAAUAmUA | | 10902779- |
| | | AUACUGGA | UGUAGCUCCCUGA | CUmGGAmGUUGmUmAm | | 10902803 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029087 | 378 | CACCAUGG | CACCAUGGUAGAU | mC*mA*mC*mCmAUGm | 2378 | chr16: |
| | | UAGAUGAA | GAAUAUACUGGGU | GmUAmGAmUGAAUmAU | | 10902780- |
| | | UAUACUGG | UGUAGCUCCCUGA | ACmUGGmGUUGmUmAm | | 10902804 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029088 | 379 | UCACCAUG | UCACCAUGGUAGA | mU*mC*mA*mCmCAUm | 2379 | chr16: |
| | | GUAGAUGA | UGAAUAUACUGGU | GmGUmAGmAUGAAmUA | | 10902781- |
| | | AUAUACUG | UGUAGCUCCCUGA | UAmCUGmGUUGmUmAm | | 10902805 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029089 | 380 | UCUACCAU | UCUACCAUGGUGA | mU*mC*mU*mAmCCAm | 2380 | chr16: |
| | | GGUGAGUG | GUGCGGGGCCUGU | UmGGmUGmAGUGCmGG | | 10902792- |
| | | CGGGGCCU | UGUAGCUCCCUGA | GGmCCUmGUUGmUmAm | | 10902816 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029090 | 381 | CUACCAUG | CUACCAUGGUGAG | mC*mU*mA*mCmCAUm | 2381 | chr16: |
| | | GUGAGUGC | UGCGGGGCCUGGU | GmGUmGAmGUGCGmGG | | 10902793- |
| | | GGGGCCUG | UGUAGCUCCCUGA | GCmCUGmGUUGmUmAm | | 10902817 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029091 | 382 | UACCAUGG | UACCAUGGUGAGU | mU*mA*mC*mCmAUGm | 2382 | chr16: |
| | | UGAGUGCG | GCGGGGCCUGGGU | GmUGmAGmUGCGGmGG | | 10902794- |
| | | GGGCCUGG | UGUAGCUCCCUGA | CCmUGGmGUUGmUmAm | | 10902818 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029092 | 383 | AUGGUGAG | AUGGUGAGUGCGG | mA*mU*mG*mGmUGAm | 2383 | chr16: |
| | | UGCGGGGC | GGCCUGGCUCCGU | GmUGmCGmGGGCCmUG | | 10902798- |
| | | CUGGCUCC | UGUAGCUCCCUGA | GCmUCCmGUUGmUmAm | | 10902822 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029093 | 384 | GUGAGUGC | GUGAGUGCGGGGC | mG*mU*mG*mAmGUGm | 2384 | chr16: |
| | | GGGGCCUG | CUGGCUCCCCGGU | CmGGmGGmCCUGGmCU | | 10902801- |
| | | GCUCCCCG | UGUAGCUCCCUGA | CCmCCGmGUUGmUmAm | | 10902825 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029094 | 385 | ACUCUCCA | ACUCUCCACCCCC | mA*mC*mU*mCmUCCm | 2385 | chr16: |
| | | CCCCCAAU | AAUGUAGGUGAGU | AmCCmCCmCAAUGmUA | | 10903710- |
| | | GUAGGUGA | UGUAGCUCCCUGA | GGmUGAmGUUGmUmAm | | 10903734 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029095 | 386 | CUCUCCAC | CUCUCCACCCCCA | mC*mU*mC*mUmCCAm | 2386 | chr16: |
| | | CCCCAAUG | AUGUAGGUGAGGU | CmCCmCCmAAUGUmAG | | 10903711- |
| | | UAGGUGAG | UGUAGCUCCCUGA | GUmGAGmGUUGmUmAm | | 10903735 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029096 | 387 | UCUCCACC | UCUCCACCCCCAA | mU*mC*mU*mCmCACm | 2387 | chr16: |
| | | CCCAAUGU | UGUAGGUGAGGGU | CmCCmCAmAUGUAmGG | | 10903712- |
| | | AGGUGAGG | UGUAGCUCCCUGA | UGmAGGmGUUGmUmAm | | 10903736 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029097 | 388 | ACCCCCAA | ACCCCCAAUGUAG | mA*mC*mC*mCmCCAm | 2388 | chr16: |
| | | UGUAGGUG | GUGAGGUGCCCGU | AmUGmUAmGGUGAmGG | | 10903717- |
| | | AGGUGCCC | UGUAGCUCCCUGA | UGmCCCmGUUGmUmAm | | 10903741 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029098 | 389 | CCAAUGUA | CCAAUGUAGGUGA | mC*mC*mA*mAmUGUm | 2389 | chr16: |
| | | GGUGAGGU | GGUGCCCCAGGGU | AmGGmUGmAGGUGmCC | | 10903721- |
| | | GCCCCAGG | UGUAGCUCCCUGA | CCmAGGmGUUGmUmAm | | 10903745 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029099 | 390 | AGGUGAGG | AGGUGAGGUGCCC | mA*mG*mG*mUmGAGm | 2390 | chr16: |
| | | UGCCCCAG | CAGGCCAGCCAGU | GmUGmCCmCCAGGmCC | | 10903728- |
| | | GCCAGCCA | UGUAGCUCCCUGA | AGmCCAmGUUGmUmAm | | 10903752 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029100 | 391 | GGUGAGGU | GGUGAGGUGCCCC | mG*mG*mU*mGmAGGm | 2391 | chr16: |
| | | GCCCCAGG | AGGCCAGCCAAGU | UmGCmCCmCAGGCmCA | | 10903729- |
| | | CCAGCCAA | UGUAGCUCCCUGA | GCmCAAmGUUGmUmAm | | 10903753 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029101 | 392 | GUGAGGUG | GUGAGGUGCCCCA | mG*mU*mG*mAmGGUm | 2392 | chr16: |
| | | CCCCAGGC | GGCCAGCCAAGGU | GmCCmCCmAGGCCmAG | | 10903730- |
| | | CAGCCAAG | UGUAGCUCCCUGA | CCmAAGmGUUGmUmAm | | 10903754 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029102 | 393 | UGAGGUGC | UGAGGUGCCCCAG | mU*mG*mA*mGmGUGm | 2393 | chr16: |
| | | CCCAGGCC | GCCAGCCAAGUGU | CmCCmCAmGGCCAmGC | | 10903731- |
| | | AGCCAAGU | UGUAGCUCCCUGA | CAmAGUmGUUGmUmAm | | 10903755 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029103 | 394 | GGUGCCCC | GGUGCCCCAGGCC | mG*mG*mU*mGmCCCm | 2394 | chr16: |
| | | AGGCCAGC | AGCCAAGUACCGU | CmAGmGCmCAGCCmAA | | 10903734- |
| | | CAAGUACC | UGUAGCUCCCUGA | GUmACCmGUUGmUmAm | | 10903758 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029104 | 395 | GUGCCCCA | GUGCCCCAGGCCA | mG*mU*mG*mCmCCCm | 2395 | chr16: |
| | | GGCCAGCC | GCCAAGUACCCGU | AmGGmCCmAGCCAmAG | | 10903735- |
| | | AAGUACCC | UGUAGCUCCCUGA | UAmCCCmGUUGmUmAm | | 10903759 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029105 | 396 | AAGUACCC | AAGUACCCCCUCC | mA*mA*mG*mUmACCm | 2396 | chr16: |
| | | CCUCCCAG | CAGUGGAUUCAGU | CmCCmUCmCCAGUmGG | | 10903751- |
| | | UGGAUUCA | UGUAGCUCCCUGA | AUmUCAmGUUGmUmAm | | 10903775 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029106 | 397 | CCUCCCGA | CCUCCCGAGCAAA | mC*mC*mU*mCmCCGm | 2397 | chr16: |
| | | GCAAACAU | CAUGACAGGUAGU | AmGCmAAmACAUGmAC | | 10903874- |
| | | GACAGGUA | UGUAGCUCCCUGA | AGmGUAmGUUGmUmAm | | 10903898 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029107 | 398 | CUCCCGAG | CUCCCGAGCAAAC | mC*mU*mC*mCmCGAm | 2398 | chr16: |
| | | CAAACAUG | AUGACAGGUAAGU | GmCAmAAmCAUGAmCA | | 10903875- |
| | | ACAGGUAA | UGUAGCUCCCUGA | GGmUAAmGUUGmUmAm | | 10903899 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029108 | 399 | CAAACAUG | CAAACAUGACAGG | mC*mA*mA*mAmCAUm | 2399 | chr16: |
| | | ACAGGUAA | UAAGGACCCUUGU | GmACmAGmGUAAGmGA | | 10903883- |
| | | GGACCCUU | UGUAGCUCCCUGA | CCmCUUmGUUGmUmAm | | 10903907 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029109 | 400 | UUGUGCUC | UUGUGCUCUGGAG | mU*mU*mG*mUmGCUm | 2400 | chr16: |
| | | UGGAGAUG | AUGGAGAAGCAGU | CmUGmGAmGAUGGmAG | | 10904726- |
| | | GAGAAGCA | UGUAGCUCCCUGA | AAmGCAmGUUGmUmAm | | 10904750 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029110 | 401 | GCUUCUCC | GCUUCUCCAUCUC | mG*mC*mU*mUmCUCm | 2401 | chr16: |
| | | AUCUCCAG | CAGAGCACAAGGU | CmAUmCUmCCAGAmGC | | 10904727- |
| | | AGCACAAG | UGUAGCUCCCUGA | ACmAAGmGUUGmUmAm | | 10904751 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029111 | 402 | CUUCUCCA | CUUCUCCAUCUCC | mC*mU*mU*mCmUCCm | 2402 | chr16: |
| | | UCUCCAGA | AGAGCACAAGAGU | AmUCmUCmCAGAGmCA | | 10904728- |
| | | GCACAAGA | UGUAGCUCCCUGA | CAmAGAmGUUGmUmAm | | 10904752 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029112 | 403 | UUCUCCAU | UUCUCCAUCUCCA | mU*mU*mC*mUmCCAm | 2403 | chr16: |
| | | CUCCAGAG | GAGCACAAGACGU | UmCUmCCmAGAGCmAC | | 10904729- |
| | | CACAAGAC | UGUAGCUCCCUGA | AAmGACmGUUGmUmAm | | 10904753 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029113 | 404 | UCUCCAUC | UCUCCAUCUCCAG | mU*mC*mU*mCmCAUm | 2404 | chr16: |
| | | UCCAGAGC | AGCACAAGACGGU | CmUCmCAmGAGCAmCA | | 10904730- |
| | | ACAAGACG | UGUAGCUCCCUGA | AGmACGmGUUGmUmAm | | 10904754 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029114 | 405 | CCAUCUCC | CCAUCUCCAGAGC | mC*mC*mA*mUmCUCm | 2405 | chr16: |
| | | AGAGCACA | ACAAGACGUCCGU | CmAGmAGmCACAAmGA | | 10904733- |
| | | AGACGUCC | UGUAGCUCCCUGA | CGmUCCmGUUGmUmAm | | 10904757 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029115 | 406 | CAUCUCCA | CAUCUCCAGAGCA | mC*mA*mU*mCmUCCm | 2406 | chr16: |
| | | GAGCACAA | CAAGACGUCCCGU | AmGAmGCmACAAGmAC | | 10904734- |
| | | GACGUCCC | UGUAGCUCCCUGA | GUmCCCmGUUGmUmAm | | 10904758 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029116 | 407 | AGAGCACA | AGAGCACAAGACG | mA*mG*mA*mGmCACm | 2407 | chr16: |
| | | AGACGUCC | UCCCCCACCCAGU | AmAGmACmGUCCCmCC | | 10904741- |
| | | CCCACCCA | UGUAGCUCCCUGA | ACmCCAmGUUGmUmAm | | 10904765 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029117 | 408 | ACCCAAUG | ACCCAAUGCCCGG | mA*mC*mC*mCmAAUm | 2408 | chr16: |
| | | CCCGGCAG | CAGCUGGAGAGGU | GmCCmCGmGCAGCmUG | | 10904760- |
| | | CUGGAGAG | UGUAGCUCCCUGA | GAmGAGmGUUGmUmAm | | 10904784 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029118 | 409 | CCAUUUUG | CCAUUUUGGAAGC | mC*mC*mA*mUmUUUm | 2409 | chr16: |
| | | GAAGCUUG | UUGUUGGAGACGU | GmGAmAGmCUUGUmUG | | 10904784- |
| | | UUGGAGAC | UGUAGCUCCCUGA | GAmGACmGUUGmUmAm | | 10904808 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029119 | 410 | CCAGGCCA | CCAGGCCAUUUUG | mC*mC*mA*mGmGCCm | 2410 | chr16: |
| | | UUUUGGAA | GAAGCUUGUUGGU | AmUUmUUmGGAAGmCU | | 10904789- |
| | | GCUUGUUG | UGUAGCUCCCUGA | UGmUUGmGUUGmUmAm | | 10904813 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029120 | 411 | GCCUGGUG | GCCUGGUGAGUGA | mG*mC*mC*mUmGGUm | 2411 | chr16: |
| | | AGUGAUGC | UGCGGGAUCUCGU | GmAGmUGmAUGCGmGG | | 10904807- |
| | | GGGAUCUC | UGUAGCUCCCUGA | AUmCUCmGUUGmUmAm | | 10904831 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029121 | 412 | CCUGGUGA | CCUGGUGAGUGAU | mC*mC*mU*mGmGUGm | 2412 | chr16: |
| | | GUGAUGCG | GCGGGAUCUCUGU | AmGUmGAmUGCGGmGA | | 10904808- |
| | | GGAUCUCU | UGUAGCUCCCUGA | UCmUCUmGUUGmUmAm | | 10904832 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029122 | 413 | CCUUUGCA | CCUUUGCAGAGCC | mC*mC*mU*mUmUGCm | 2413 | chr16: |
| | | GAGCCGGU | GGUGGAGCAGUGU | AmGAmGCmCGGUGmGA | | 10906489- |
| | | GGAGCAGU | UGUAGCUCCCUGA | GCmAGUmGUUGmUmAm | | 10906513 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029123 | 414 | UAGAACUG | UAGAACUGCUCCA | mU*mA*mG*mAmACUm | 2414 | chr16: |
| | | CUCCACCG | CCGGCUCUGCAGU | GmCUmCCmACCGGmCU | | 10906493- |
| | | GCUCUGCA | UGUAGCUCCCUGA | CUmGCAmGUUGmUmAm | | 10906517 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029124 | 415 | UACCGCUC | UACCGCUCACUGC | mU*mA*mC*mCmGCUm | 2415 | chr16: |
| | | ACUGCAGG | AGGACACGUAUGU | CmACmUGmCAGGAmCA | | 10906515- |
| | | ACACGUAU | UGUAGCUCCCUGA | CGmUAUmGUUGmUmAm | | 10906539 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029125 | 416 | CUCACUGC | CUCACUGCAGGAC | mC*mU*mC*mAmCUGm | 2416 | chr16: |
| | | AGGACACG | ACGUAUGGUGCGU | CmAGmGAmCACGUmAU | | 10906520- |
| | | UAUGGUGC | UGUAGCUCCCUGA | GGmUGCmGUUGmUmAm | | 10906544 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029126 | 417 | UCACUGCA | UCACUGCAGGACA | mU*mC*mA*mCmUGCm | 2417 | chr16: |
| | | GGACACGU | CGUAUGGUGCCGU | AmGGmACmACGUAmUG | | 10906521- |
| | | AUGGUGCC | UGUAGCUCCCUGA | GUmGCCmGUUGmUmAm | | 10906545 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029127 | 418 | AGGACACG | AGGACACGUAUGG | mA*mG*mG*mAmCACm | 2418 | chr16: |
| | | UAUGGUGC | UGCCGAGCCCGGU | GmUAmUGmGUGCCmGA | | 10906528- |
| | | CGAGCCCG | UGUAGCUCCCUGA | GCmCCGmGUUGmUmAm | | 10906552 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029128 | 419 | GCCCAGUC | GCCCAGUCCGGGG | mG*mC*mC*mCmAGUm | 2419 | chr16: |
| | | CGGGGUGG | UGGCCAGUUCCGU | CmCGmGGmGUGGCmCA | | 10906631- |
| | | CCAGUUCC | UGUAGCUCCCUGA | GUmUCCmGUUGmUmAm | | 10906655 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029129 | 420 | GUUCUGCO | GUUCUGCCCAGUC | mG*mU*mU*mCmUGCm | 2420 | chr16: |
| | | CAGUCCGG | CGGGGUGGCCAGU | CmCAmGUmCCGGGmGU | | 10906636- |
| | | GGUGGCCA | UGUAGCUCCCUGA | GGmCCAmGUUGmUmAm | | 10906660 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029130 | 421 | CGUUCUGC | CGUUCUGCCCAGU | mC*mG*mU*mUmCUGm | 2421 | chr16: |
| | | CCAGUCCG | CCGGGGUGGCCGU | CmCCmAGmUCCGGmGG | | 10906637- |
| | | GGGUGGCC | UGUAGCUCCCUGA | UGmGCCmGUUGmUmAm | | 10906661 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029131 | 422 | AGCUGCCG | AGCUGCCGUUCUG | mA*mG*mC*mUmGCCm | 2422 | chr16: |
| | | UUCUGCCC | CCCAGUCCGGGGU | GmUUmCUmGCCCAmGU | | 10906643- |
| | | AGUCCGGG | UGUAGCUCCCUGA | CCmGGGmGUUGmUmAm | | 10906667 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029132 | 423 | GGGCAGAA | GGGCAGAACGGCA | mG*mG*mG*mCmAGAm | 2423 | chr16: |
| | | CGGCAGCU | GCUGGCCCAAGGU | AmCGmGCmAGCUGmGC | | 10906651- |
| | | GGCCCAAG | UGUAGCUCCCUGA | CCmAAGmGUUGmUmAm | | 10906675 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029133 | 424 | CAAUAGCU | CAAUAGCUCUUGC | mC*mA*mA*mUmAGCm | 2424 | chr16: |
| | | CUUGCCCU | CCUGACCAGCUGU | UmCUmUGmCCCUGmAC | | 10906754- |
| | | GACCAGCU | UGUAGCUCCCUGA | CAmGCUmGUUGmUmAm | | 10906778 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029134 | 425 | CCAAUAGC | CCAAUAGCUCUUG | mC*mC*mA*mAmUAGm | 2425 | chr16: |
| | | UCUUGCCC | CCCUGACCAGCGU | CmUCmUUmGCCCUmGA | | 10906755- |
| | | UGACCAGC | UGUAGCUCCCUGA | CCmAGCmGUUGmUmAm | | 10906779 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029135 | 426 | UGCCCCAG | UGCCCCAGCCCAA | mU*mG*mC*mCmCCAm | 2426 | chr16: |
| | | CCCAAUAG | UAGCUCUUGCCGU | GmCCmCAmAUAGCmUC | | 10906764- |
| | | CUCUUGCC | UGUAGCUCCCUGA | UUmGCCmGUUGmUmAm | | 10906788 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029136 | 427 | CUCACUGC | CUCACUGCCCCAG | mC*mU*mC*mAmCUGm | 2427 | chr16: |
| | | CCCAGCCC | CCCAAUAGCUCGU | CmCCmCAmGCCCAmAU | | 10906769- |
| | | AAUAGCUC | UGUAGCUCCCUGA | AGmCUCmGUUGmUmAm | | 10906793 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029137 | 428 | GCUCACUG | GCUCACUGCCCCA | mG*mC*mU*mCmACUm | 2428 | chr16: |
| | | CCCCAGCC | GCCCAAUAGCUGU | GmCCmCCmAGCCCmAA | | 10906770- |
| | | CAAUAGCU | UGUAGCUCCCUGA | UAmGCUmGUUGmUmAm | | 10906794 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029138 | 429 | AAGCCCAG | AAGCCCAGGCCCG | mA*mA*mG*mCmCCAm | 2429 | chr16: |
| | | GCCCGGCU | GCUCACUGCCCGU | GmGCmCCmGGCUCmAC | | 91006783- |
| | | CACUGCCC | UGUAGCUCCCUGA | UGmCCCmGUUGmUmAm | | 10906807 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029139 | 430 | CAAGCCCA | CAAGCCCAGGCCC | mC*mA*mA*mGmCCCm | 2430 | chr16: |
| | | GGCCCGGC | GGCUCACUGCCGU | AmGGmCCmCGGCUmCA | | 10906784- |
| | | UCACUGCC | UGUAGCUCCCUGA | CUmGCCmGUUGmUmAm | | 10906808 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029140 | 431 | GCCACAAG | GCCACAAGCCCAG | mG*mC*mC*mAmCAAm | 2431 | chr16: |
| | | CCCAGGCC | GCCCGGCUCACGU | GmCCmCAmGGCCCmGG | | 10906788- |
| | | CGGCUCAC | UGUAGCUCCCUGA | CUmCACmGUUGmUmAm | | 10906812 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029141 | 432 | GGCCACAA | GGCCACAAGCCCA | mG*mG*mC*mCmACAm | 2432 | chr16: |
| | | GCCCAGGC | GGCCCGGCUCAGU | AmGCmCCmAGGCCmCG | | 10906789- |
| | | CCGGCUCA | UGUAGCUCCCUGA | GCmUCAmGUUGmUmAm | | 10906813 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029142 | 433 | CGGCCACA | CGGCCACAAGCCC | mC*mG*mG*mCmCACm | 2433 | chr16: |
| | | AGCCCAGG | AGGCCCGGCUCGU | AmAGmCCmCAGGCmCC | | 10906790- |
| | | CCCGGCUC | UGUAGCUCCCUGA | GGmCUCmGUUGmUmAm | | 10906814 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029143 | 434 | UUCCCCAG | UUCCCCAGUACGA | mU*mU*mC*mCmCCAm | 2434 | chr16: |
| | | UACGACUU | CUUUGUCUUCUGU | GmUAmCGmACUUUmGU | | 10906816- |
| | | UGUCUUCU | UGUAGCUCCCUGA | CUmUCUmGUUGmUmAm | | 10906840 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029144 | 435 | UCCCCAGU | UCCCCAGUACGAC | mU*mC*mC*mCmCAGm | 2435 | chr16: |
| | | ACGACUUU | UUUGUCUUCUCGU | UmACmGAmCUUUGmUC | | 10906817- |
| | | GUCUUCUC | UGUAGCUCCCUGA | UUmCUCmGUUGmUmAm | | 10906841 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029145 | 436 | CCCCAGUA | CCCCAGUACGACU | mC*mC*mC*mCmAGUm | 2436 | chr16: |
| | | CGACUUUG | UUGUCUUCUCUGU | AmCGmACmUUUGUmCU | | 10906818- |
| | | UCUUCUCU | UGUAGCUCCCUGA | UCmUCUmGUUGmUmAm | | 10906842 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029146 | 437 | AGUACGAC | AGUACGACUUUGU | mA*mG*mU*mAmCGAm | 2437 | chr16: |
| | | UUUGUCUU | CUUCUCUGUCCGU | CmUUmUGmUCUUCmUC | | 10906822- |
| | | CUCUGUCC | UGUAGCUCCCUGA | UGmUCCmGUUGmUmAm | | 10906846 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029147 | 438 | GCCUAUGG | GCCUAUGGCCUGC | mG*mC*mC*mUmAUGm | 2438 | chr16: |
| | | CCUGCAGG | AGGAUCUGCUCGU | GmCCmUGmCAGGAmUC | | 10906875- |
| | | AUCUGCUC | UGUAGCUCCCUGA | UGmCUCmGUUGmUmAm | | 10906899 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029148 | 439 | CCUAUGGC | CCUAUGGCCUGCA | mC*mC*mU*mAmUGGm | 2439 | chr16: |
| | | CUGCAGGA | GGAUCUGCUCUGU | CmCUmGCmAGGAUmCU | | 10906876- |
| | | UCUGCUCU | UGUAGCUCCCUGA | GCmUCUmGUUGmUmAm | | 10906900 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029149 | 440 | GCCUGCAG | GCCUGCAGGAUCU | mG*mC*mC*mUmGCAm | 2440 | chr16: |
| | | GAUCUGCU | GCUCUUCUCCCGU | GmGAmUCmUGCUCmUU | | 10906882- |
| | | CUUCUCCC | UGUAGCUCCCUGA | CUmCCCmGUUGmUmAm | | 10906906 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029150 | 441 | CCUGCAGG | CCUGCAGGAUCUG | mC*mC*mU*mGmCAGm | 2441 | chr16: |
| | | AUCUGCUC | CUCUUCUCCCUGU | GmAUmCUmGCUCUmUC | | 10906883- |
| | | UUCUCCCU | UGUAGCUCCCUGA | UCmCCUmGUUGmUmAm | | 10906907 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029151 | 442 | UCCCUGGG | UCCCUGGGCCCAC | mU*mC*mC*mCmUGGm | 2442 | chr16: |
| | | CCCACAGC | AGCCACUCGUGGU | GmCCmCAmCAGCCmAC | | 10906902- |
| | | CACUCGUG | UGUAGCUCCCUGA | UCmGUGmGUUGmUmAm | | 10906926 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029152 | 443 | UCGAGGAG | UCGAGGAGCUGGA | mU*mC*mG*mAmGGAm | 2443 | chr16: |
| | | CUGGAAGC | AGCGCAAGAUGGU | GmCUmGGmAAGCGmCA | | 10906993- |
| | | GCAAGAUG | UGUAGCUCCCUGA | AGmAUGmGUUGmUmAm | | 10907017 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029153 | 444 | UGGCCGGC | UGGCCGGCCUUUU | mU*mG*mG*mCmCGGm | 2444 | chr16: |
| | | CUUUUCCA | CCAGAAGAAGCGU | CmCUmUUmUCCAGmAA | | 10907077- |
| | | GAAGAAGC | UGUAGCUCCCUGA | GAmAGCmGUUGmUmAm | | 10907101 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029154 | 445 | UUCCAGAA | UUCCAGAAGAAGC | mU*mU*mC*mCmAGAm | 2445 | chr16: |
| | | GAAGCUGC | UGCUCCGAGGUGU | AmGAmAGmCUGCUmCC | | 10907088- |
| | | UCCGAGGU | UGUAGCUCCCUGA | GAmGGUmGUUGmUmAm | | 10907112 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029155 | 446 | UCCAGAAG | UCCAGAAGAAGCU | mU*mC*mC*mAmGAAm | 2446 | chr16: |
| | | AAGCUGCU | GCUCCGAGGUUGU | GmAAmGCmUGCUCmCG | | 10907089- |
| | | CCGAGGUU | UGUAGCUCCCUGA | AGmGUUmGUUGmUmAm | | 10907113 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029156 | 447 | AGAAGAAG | AGAAGAAGCUGCU | mA*mG*mA*mAmGAAm | 2447 | chr16: |
| | | CUGCUCCG | CCGAGGUUGCAGU | GmCUmGCmUCCGAmGG | | 10907092- |
| | | AGGUUGCA | UGUAGCUCCCUGA | UUmGCAmGUUGmUmAm | | 10907116 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029157 | 448 | AGAAGCUG | AGAAGCUGCUCCG | mA*mG*mA*mAmGCUm | 2448 | chr16: |
| | | CUCCGAGG | AGGUUGCACCCGU | GmCUmCCmGAGGUmUG | | 10907095- |
| | | UUGCACCC | UGUAGCUCCCUGA | CAmCCCmGUUGmUmAm | | 10907119 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029158 | 449 | CUCCGAGG | CUCCGAGGUUGCA | mC*mU*mC*mCmGAGm | 2449 | chr16: |
| | | UUGCACCC | CCCUCCUCCUCGU | GmUUmGCmACCCUmCC | | 10907103- |
| | | UCCUCCUC | UGUAGCUCCCUGA | UCmCUCmGUUGmUmAm | | 10907127 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029159 | 450 | UCCGAGGU | UCCGAGGUUGCAC | mU*mC*mC*mGmAGGm | 2450 | chr16: |
| | | UGCACCCU | CCUCCUCCUCAGU | UmUGmCAmCCCUCmCU | | 10907104- |
| | | CCUCCUCA | UGUAGCUCCCUGA | CCmUCAmGUUGmUmAm | | 10907128 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029160 | 451 | CUGGUCCA | CUGGUCCAGAGCC | mC*mU*mG*mGmUCCm | 2451 | chr16: |
| | | GAGCCUGA | UGAGCAAGGCCGU | AmGAmGCmCUGAGmCA | | 10907148- |
| | | GCAAGGCC | UGUAGCUCCCUGA | AGmGCCmGUUGmUmAm | | 10907172 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029161 | 452 | UGGUCCAG | UGGUCCAGAGCCU | mU*mG*mG*mUmCCAm | 2452 | chr16: |
| | | AGCCUGAG | GAGCAAGGCCGGU | GmAGmCCmUGAGCmAA | | 10907149- |
| | | CAAGGCCG | UGUAGCUCCCUGA | GGmCCGmGUUGmUmAm | | 10907173 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029162 | 453 | UCAGGGAU | UCAGGGAUGACAG | mU*mC*mA*mGmGGAm | 2453 | chr16: |
| | | GACAGAGC | AGCACCAAGACGU | UmGAmCAmGAGCAmCC | | 10907244- |
| | | ACCAAGAC | UGUAGCUCCCUGA | AAmGACmGUUGmUmAm | | 10907268 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029163 | 454 | CAGGGAUG | CAGGGAUGACAGA | mC*mA*mG*mGmGAUm | 2454 | chr16: |
| | | ACAGAGCA | GCACCAAGACAGU | GmACmAGmAGCACmCA | | 10907245- |
| | | CCAAGACA | UGUAGCUCCCUGA | AGmACAmGUUGmUmAm | | 10907269 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029164 | 455 | CAGAGCAC | CAGAGCACCAAGA | mC*mA*mG*mAmGCAm | 2455 | chr16: |
| | | CAAGACAG | CAGAGCCCUGAGU | CmCAmAGmACAGAmGC | | 10907254- |
| | | AGCCCUGA | UGUAGCUCCCUGA | CCmUGAmGUUGmUmAm | | 10907278 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029165 | 456 | AGCACCAA | AGCACCAAGACAG | mA*mG*mC*mAmCCAm | 2456 | chr16: |
| | | GACAGAGC | AGCCCUGACGCGU | AmGAmCAmGAGCCmCU | | 10907257- |
| | | CCUGACGC | UGUAGCUCCCUGA | GAmCGCmGUUGmUmAm | | 10907281 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029166 | 457 | AAGACAGA | AAGACAGAGCCCU | mA*mA*mG*mAmCAGm | 2457 | chr16: |
| | | GCCCUGAC | GACGCUCCUCCGU | AmGCmCCmUGACGmCU | | 10907263- |
| | | GCUCCUCC | UGUAGCUCCCUGA | CCmUCCmGUUGmUmAm | | 10907287 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029167 | 458 | UGCCGGGC | UGCCGGGCAGUGU | mU*mG*mC*mCmGGGm | 2458 | chr16: |
| | | AGUGUGCC | GCCAGCUCUCAGU | CmAGmUGmUGCCAmGC | | 10907331- |
| | | AGCUCUCA | UGUAGCUCCCUGA | UCmUCAmGUUGmUmAm | | 10907355 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029168 | 459 | GCCGGGCA | GCCGGGCAGUGUG | mG*mC*mC*mGmGGCm | 2459 | chr16: |
| | | GUGUGCCA | CCAGCUCUCAGGU | AmGUmGUmGCCAGmCU | | 10907332- |
| | | GCUCUCAG | UGUAGCUCCCUGA | CUmCAGmGUUGmUmAm | | 10907356 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029169 | 460 | CAGGCCAG | CAGGCCAGCUUGG | mC*mA*mG*mGmCCAm | 2460 | chr16: |
| | | CUUGGCCA | CCAGCUCUGCCGU | GmCUmUGmGCCAGmCU | | 10907462- |
| | | GCUCUGCC | UGUAGCUCCCUGA | CUmGCCmGUUGmUmAm | | 10907486 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029170 | 461 | AGCUCCCA | AGCUCCCAGGCCA | mA*mG*mC*mUmCCCm | 2461 | chr16: |
| | | GGCCAGCU | GCUUGGCCAGCGU | AmGGmCCmAGCUUmGG | | 10907468- |
| | | UGGCCAGC | UGUAGCUCCCUGA | CCmAGCmGUUGmUmAm | | 10907492 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029171 | 462 | CUGCGGCC | CUGCGGCCCAGCU | mC*mU*mG*mCmGGCm | 2462 | chr16: |
| | | CAGCUCCC | CCCAGGCCAGCGU | CmCAmGCmUCCCAmGG | | 10907477- |
| | | AGGCCAGC | UGUAGCUCCCUGA | CCmAGCmGUUGmUmAm | | 10907501 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029172 | 463 | GCAGACAU | GCAGACAUCAAAG | mG*mC*mA*mGmACAm | 2463 | chr16: |
| | | CAAAGUAC | UACCCUACAGGGU | UmCAmAAmGUACCmCU | | 10907497- |
| | | CCUACAGG | UGUAGCUCCCUGA | ACmAGGmGUUGmUmAm | | 10907521 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029173 | 464 | AUCAAAGU | AUCAAAGUACCCU | mA*mU*mC*mAmAAGm | 2464 | chr16: |
| | | ACCCUACA | ACAGGAGGACCGU | UmACmCCmUACAGmGA | | 10907503- |
| | | GGAGGACC | UGUAGCUCCCUGA | GGmACCmGUUGmUmAm | | 10907527 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029174 | 465 | GACCAGUU | GACCAGUUCCCAU | mG*mA*mC*mCmAGUm | 2465 | chr16: |
| | | CCCAUCCG | CCGCAGACGUGGU | UmCCmCAmUCCGCmAG | | 10907523- |
| | | CAGACGUG | UGUAGCUCCCUGA | ACmGUGmGUUGmUmAm | | 10907547 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029175 | 466 | GAUGGCCA | GAUGGCCAAAGGC | mG*mA*mU*mGmGCCm | 2466 | chr16: |
| | | AAGGCUUA | UUAGUCCAACAGU | AmAAmGGmCUUAGmUC | | 91007558- |
| | | GUCCAACA | UGUAGCUCCCUGA | CAmACAmGUUGmUmAm | | 10907582 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029176 | 467 | AAAGGCUU | AAAGGCUUAGUCC | mA*mA*mA*mGmGCUm | 2467 | chr16: |
| | | AGUCCAAC | AACACCCACCGGU | UmAGmUCmCAACAmCC | | 10907565- |
| | | ACCCACCG | UGUAGCUCCCUGA | CAmCCGmGUUGmUmAm | | 10907589 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029177 | 468 | GUCCAACA | GUCCAACACCCAC | mG*mU*mC*mCmAACm | 2468 | chr16: |
| | | CCCACCGC | CGCGGGCCGCAGU | AmCCmCAmCCGCGmGG | | 10907574- |
| | | GGGCCGCA | UGUAGCUCCCUGA | CCmGCAmGUUGmUmAm | | 10907598 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029178 | 469 | AGCUUCCU | AGCUUCCUCCUGC | mA*mG*mC*mUmUCCm | 2469 | chr16: |
| | | CCUGCAAU | AAUGCUUCCUGGU | UmCCmUGmCAAUGmCU | | 10907619- |
| | | GCUUCCUG | UGUAGCUCCCUGA | UCmCUGmGUUGmUmAm | | 10907643 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029179 | 470 | GCUUCCUC | GCUUCCUCCUGCA | mG*mC*mU*mUmCCUm | 2470 | chr16: |
| | | CUGCAAUG | AUGCUUCCUGGGU | CmCUmGCmAAUGCmUU | | 10907620- |
| | | CUUCCUGG | UGUAGCUCCCUGA | CCmUGGmGUUGmUmAm | | 10907644 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029180 | 471 | UCGCCACU | UCGCCACUCAGAG | mU*mC*mG*mCmCACm | 2471 | chr16: |
| | | CAGAGCCA | CCAGCCACAGGGU | UmCAmGAmGCCAGmCC | | 10907648- |
| | | GCCACAGG | UGUAGCUCCCUGA | ACmAGGmGUUGmUmAm | | 10907672 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029181 | 472 | UUCGCCAC | UUCGCCACUCAGA | mU*mU*mC*mGmCCAm | 2472 | chr16: |
| | | UCAGAGCC | GCCAGCCACAGGU | CmUCmAGmAGCCAmGC | | 10907649- |
| | | AGCCACAG | UGUAGCUCCCUGA | CAmCAGmGUUGmUmAm | | 10907673 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029182 | 473 | UUUCGCCA | UUUCGCCACUCAG | mU*mU*mU*mCmGCCm | 2473 | chr16: |
| | | CUCAGAGC | AGCCAGCCACAGU | AmCUmCAmGAGCCmAG | | 10907650- |
| | | CAGCCACA | UGUAGCUCCCUGA | CCmACAmGUUGmUmAm | | 10907674 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029183 | 474 | AUUUCGCC | AUUUCGCCACUCA | mA*mU*mU*mUmCGCm | 2474 | chr16: |
| | | ACUCAGAG | GAGCCAGCCACGU | CmACmUCmAGAGCmCA | | 10907651- |
| | | CCAGCCAC | UGUAGCUCCCUGA | GCmCACmGUUGmUmAm | | 10907675 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029184 | 475 | AAGGAGCU | AAGGAGCUCCCGC | mA*mA*mG*mGmAGCm | 2475 | chr16: |
| | | CCCGCAGU | AGUACCUAGCAGU | UmCCmCGmCAGUAmCC | | 10907682- |
| | | ACCUAGCA | UGUAGCUCCCUGA | UAmGCAmGUUGmUmAm | | 10907706 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029185 | 476 | AGGAGCUC | AGGAGCUCCCGCA | mA*mG*mG*mAmGCUm | 2476 | chr16: |
| | | CCGCAGUA | GUACCUAGCAUGU | CmCCmGCmAGUACmCU | | 10907683- |
| | | CCUAGCAU | UGUAGCUCCCUGA | AGmCAUmGUUGmUmAm | | 10907707 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029186 | 477 | GGAGCUCC | GGAGCUCCCGCAG | mG*mG*mA*mGmCUCm | 2477 | chr16: |
| | | CGCAGUAC | UACCUAGCAUUGU | CmCGmCAmGUACCmUA | | 10907684- |
| | | CUAGCAUU | UGUAGCUCCCUGA | GCmAUUmGUUGmUmAm | | 10907708 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029187 | 478 | CCAGCCAG | CCAGCCAGUUGUC | mC*mC*mA*mGmCCAm | 2478 | chr16: |
| | | UUGUCAUA | AUAGGGCCUCUGU | GmUUmGUmCAUAGmGG | | 10907722- |
| | | GGGCCUCU | UGUAGCUCCCUGA | CCmUCUmGUUGmUmAm | | 10907746 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029188 | 479 | GCACGCCC | GCACGCCCUCCAG | mG*mC*mA*mCmGCCm | 2479 | chr16: |
| | | UCCAGCCA | CCAGUUGUCAUGU | CmUCmCAmGCCAGmUU | | 10907731- |
| | | GUUGUCAU | UGUAGCUCCCUGA | GUmCAUmGUUGmUmAm | | 10907755 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029189 | 480 | CUGGCUGG | CUGGCUGGGCUGA | mC*mU*mG*mGmCUGm | 2480 | chr16: |
| | | GCUGAUCU | UCUUCCAGCCUGU | GmGCmUGmAUCUUmCC | | 91007763- |
| | | UCCAGCCU | UGUAGCUCCCUGA | AGmCCUmGUUGmUmAm | | 10907787 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G029190 | 481 | UGGCUGGG | UGGCUGGGCUGAU | mU*mG*mG*mCmUGGm | 2481 | chr16: |
| | | CUGAUCUU | CUUCCAGCCUCGU | GmCUmGAmUCUUCmCA | | 10907764- |
| | | CCAGCCUC | UGUAGCUCCCUGA | GCmCUCmGUUGmUmAm | | 10907788 |
| | | | AACCGUUGCUACA | GmCUCCCmUmGmAmAm | | |
| | | | AUAAGGCCGUCGA | AmCmCGUUmGmCUAmC | | |
| | | | AAGAUGUGCCGCA | AAU*AAGmGmCCmGmU | | |
| | | | ACGCUCUGCCUUC | mCmGmAmAmAmGmAmU | | |
| | | | UGGCAUCGUU | GUGCmCGmCAAmCGCU | | |
| | | | | CUmGmCCmUmUmCmUG | | |
| | | | | GCAUCG*mU*mU | | |
| G023421 | 482 | UACCGCUC | UACCGCUCACUGC | mU*mA*mC*mCmGCUm | 2482, | chr16: |
| | | ACUGCAGG | AGGACACGUAUGU | CmACmUGmCAGGAmCA | 3016 | 10906515- |
| | | ACACGUAU | UGUAGCUCCCUCC | CGmUAUmGUUGmUmAm | and | 10906539 |
| | | | GUUGCUACAAUAA | GmCUCCCmU(L1)mCm | 3111 | |
| | | | GGCCGUCGAUGUG | CGUUmGmCUAmCAAU* | | |
| | | | CCGCAACGCUCUG | AAGmGmCCmGmUmC(L | | |
| | | | CCGGCAUCGUU | 1)mGmAmUGUGCmCGm | | |
| | | | | CAAmCGCUCUmGmCC( | | |
| | | | | L1)GGCAUCGmU*mU | | |
| G023422 | 483 | CUCACUGC | CUCACUGCAGGAC | mC*mU*mC*mAmCUGm | 2483, | chr16: |
| | | AGGACACG | ACGUAUGGUGCGU | CmAGmGAmCACGUmAU | 3017 | 10906520- |
| | | UAUGGUGC | UGUAGCUCCCUCC | GGmUGCmGUUGmUmAm | and | 10906544 |
| | | | GUUGCUACAAUAA | GmCUCCCmU(L1)mCm | 3112 | |
| | | | GGCCGUCGAUGUG | CGUUmGmCUAmCAAU* | | |
| | | | CCGCAACGCUCUG | AAGmGmCCmGmUmC(L | | |
| | | | CCGGCAUCGUU | 1)mGmAmUGUGCmCGm | | |
| | | | | CAAmCGCUCUmGmCC( | | |
| | | | | L1)GGCAUCGmU*mU | | |
| G023423 | 484 | UCACUGCA | UCACUGCAGGACA | mU*mC*mA*mCmUGCm | 2484, | chr16: |
| | | GGACACGU | CGUAUGGUGCCGU | AmGGmACmACGUAmUG | 3018 | 10906521- |
| | | AUGGUGCC | UGUAGCUCCCUCC | GUmGCCmGUUGmUmAm | and | 10906545 |
| | | | GUUGCUACAAUAA | GmCUCCCmU(L1)mCm | 3113 | |
| | | | GGCCGUCGAUGUG | CGUUmGmCUAmCAAU* | | |
| | | | CCGCAACGCUCUG | AAGmGmCCmGmUmC(L | | |
| | | | CCGGCAUCGUU | 1)mGmAmUGUGCmCGm | | |
| | | | | CAAmCGCUCUmGmCC( | | |
| | | | | L1)GGCAUCGmU*mU | | |
| G023424 | 485 | AGGACACG | AGGACACGUAUGG | mA*mG*mG*mAmCACm | 2485, | chr16: |
| | | UAUGGUGC | UGCCGAGCCCGGU | GmUAmUGmGUGCCmGA | 3019 | 10906528- |
| | | CGAGCCCG | UGUAGCUCCCUCC | GCmCCGmGUUGmUmAm | and | 10906552 |
| | | | GUUGCUACAAUAA | GmCUCCCmU(L1)mCm | 3114 | |
| | | | GGCCGUCGAUGUG | CGUUmGmCUAmCAAU* | | |
| | | | CCGCAACGCUCUG | AAGmGmCCmGmUmC(L | | |
| | | | CCGGCAUCGUU | 1)mGmAmUGUGCmCGm | | |
| | | | | CAAmCGCUCUmGmCC( | | |
| | | | | L1)GGCAUCGmU*mU | | |
| G023425 | 486 | GGACACGU | GGACACGUAUGGU | mG*mG*mA*mCmACGm | 2486, | chr16: |
| | | AUGGUGCC | GCCGAGCCCGCGU | UmAUmGGmUGCCGmAG | 3020 | 10906529- |
| | | GAGCCCGC | UGUAGCUCCCUCC | CCmCGCmGUUGmUmAm | and | 10906553 |
| | | | GUUGCUACAAUAA | GmCUCCCmU(L1)mCm | 3115 | |
| | | | GGCCGUCGAUGUG | CGUUmGmCUAmCAAU* | | |
| | | | CCGCAACGCUCUG | AAGmGmCCmGmUmC(L | | |
| | | | CCGGCAUCGUU | 1)mGmAmUGUGCmCGm | | |
| | | | | CAAmCGCUCUmGmCC( | | |
| | | | | L1)GGCAUCGmU*mU | | |
| G023426 | 487 | AUCCUAGU | AUCCUAGUGGAGG | mA*mU*mC*mCmUAGm | 2487, | chr16: |
| | | GGAGGUGG | UGGAUCUGGUGGU | UmGGmAGmGUGGAmUC | 3021 | 10906566- |
| | | AUCUGGUG | UGUAGCUCCCUCC | UGmGUGmGUUGmUmAm | and | 10906590 |
| | | | GUUGCUACAAUAA | GmCUCCCmU(L1)mCm | 3116 | |
| | | | GGCCGUCGAUGUG | CGUUmGmCUAmCAAU* | | |
| | | | CCGCAACGCUCUG | AAGmGmCCmGmUmC(L | | |
| | | | CCGGCAUCGUU | 1)mGmAmUGUGCmCGm | | |
| | | | | CAAmCGCUCUmGmCC( | | |
| | | | | L1)GGCAUCGmU*mU | | |
| G023427 | 488 | GCAAGAGC | GCAAGAGCCUGGA | mG*mC*mA*mAmGAGm | 2488, | chr16: |
| | | CUGGAGCG | GCGGGAACUGGGU | CmCUmGGmAGCGGmGA | 3022 | 10906615- |
| | | GGAACUGG | UGUAGCUCCCUCC | ACmUGGmGUUGmUmAm | and | 10906639 |
| | | | GUUGCUACAAUAA | GmCUCCCmU(L1)mCm | 3117 | |
| | | | GGCCGUCGAUGUG | CGUUmGmCUAmCAAU* | | |
| | | | CCGCAACGCUCUG | AAGmGmCCmGmUmC(L | | |
| | | | CCGGCAUCGUU | 1)mGmAmUGUGCmCGm | | |
| | | | | CAAmCGCUCUmGmCC( | | |
| | | | | L1)GGCAUCGmU*mU | | |
| G023428 | 489 | CAAGAGCC | CAAGAGCCUGGAG | mC*mA*mA*mGmAGCm | 2489, | chr16: |
| | | UGGAGCGG | CGGGAACUGGCGU | CmUGmGAmGCGGGmAA | 3023 | 10906616- |
| | | GAACUGGC | UGUAGCUCCCUCC | CUmGGCmGUUGmUmAm | and | 10906640 |
| | | | GUUGCUACAAUAA | GmCUCCCmU(L1)mCm | 3118 | |
| | | | GGCCGUCGAUGUG | CGUUmGmCUAmCAAU* | | |
| | | | CCGCAACGCUCUG | AAGmGmCCmGmUmC(L | | |
| | | | CCGGCAUCGUU | 1)mGmAmUGUGCmCGm | | |
| | | | | CAAmCGCUCUmGmCC( | | |
| | | | | L1)GGCAUCGmU*mU | | |
| G023429 | 490 | GGGCAGAA | GGGCAGAACGGCA | mG*mG*mG*mCmAGAm | 2490, | chr16: |
| | | CGGCAGCU | GCUGGCCCAAGGU | AmCGmGCmAGCUGmGC | 3024 | 10906651- |
| | | GGCCCAAG | UGUAGCUCCCUCC | CCmAAGmGUUGmUmAm | and | 10906675 |
| | | | GUUGCUACAAUAA | GmCUCCCmU(L1)mCm | 3119 | |
| | | | GGCCGUCGAUGUG | CGUUmGmCUAmCAAU* | | |
| | | | CCGCAACGCUCUG | AAGmGmCCmGmUmC(L | | |
| | | | CCGGCAUCGUU | 1)mGmAmUGUGCmCGm | | |
| | | | | CAAmCGCUCUmGmCC( | | |
| | | | | L1)GGCAUCGmU*mU | | |
| G023430 | 491 | GCACAGCA | GCACAGCAAUCAC | mG*mC*mA*mCmAGCm | 2491, | chr16: |
| | | AUCACUCG | UCGUGUCUCACGU | AmAUmCAmCUCGUmGU | 3025 | 10906723- |
| | | UGUCUCAC | UGUAGCUCCCUCC | CUmCACmGUUGmUmAm | and | 10906747 |
| | | | GUUGCUACAAUAA | GmCUCCCmU(L1)mCm | 3120 | |
| | | | GGCCGUCGAUGUG | CGUUmGmCUAmCAAU* | | |
| | | | CCGCAACGCUCUG | AAGmGmCCmGmUmC(L | | |
| | | | CCGGCAUCGUU | 1)mGmAmUGUGCmCGm | | |
| | | | | CAAmCGCUCUmGmCC( | | |
| | | | | L1)GGCAUCGmU*mU | | |
| G023431 | 492 | CAAUAGCU | CAAUAGCUCUUGC | mC*mA*mA*mUmAGCm | 2492, | chr16: |
| | | CUUGCCCU | CCUGACCAGCUGU | UmCUmUGmCCCUGmAC | 3026 | 10906754- |
| | | GACCAGCU | UGUAGCUCCCUCC | CAmGCUmGUUGmUmAm | and | 10906778 |
| | | | GUUGCUACAAUAA | GmCUCCCmU(L1)mCm | 3121 | |
| | | | GGCCGUCGAUGUG | CGUUmGmCUAmCAAU* | | |
| | | | CCGCAACGCUCUG | AAGmGmCCmGmUmC(L | | |
| | | | CCGGCAUCGUU | 1)mGmAmUGUGCmCGm | | |
| | | | | CAAmCGCUCUmGmCC( | | |
| | | | | L1)GGCAUCGmU*mU | | |
| G023432 | 493 | CCAAUAGC | CCAAUAGCUCUUG | mC*mC*mA*mAmUAGm | 2493, | chr16: |
| | | UCUUGCCC | CCCUGACCAGCGU | CmUCmUUmGCCCUmGA | 3027 | 10906755- |
| | | UGACCAGC | UGUAGCUCCCUCC | CCmAGCmGUUGmUmAm | and | 10906779 |
| | | | GUUGCUACAAUAA | GmCUCCCmU(L1)mCm | 3122 | |
| | | | GGCCGUCGAUGUG | CGUUmGmCUAmCAAU* | | |
| | | | CCGCAACGCUCUG | AAGmGmCCmGmUmC(L | | |
| | | | CCGGCAUCGUU | 1)mGmAmUGUGCmCGm | | |
| | | | | CAAmCGCUCUmGmCC( | | |
| | | | | L1)GGCAUCGmU*mU | | |
| G023433 | 494 | AGCCGGGC | AGCCGGGCCUGGG | mA*mG*mC*mCmGGGm | 2494, | chr16: |
| | | CUGGGCUU | CUUGUGGCCGGGU | CmCUmGGmGCUUGmUG | 3028 | 10906791- |
| | | GUGGCCGG | UGUAGCUCCCUCC | GCmCGGmGUUGmUmAm | and | 10906815 |
| | | | GUUGCUACAAUAA | GmCUCCCmU(L1)mCm | 3123 | |
| | | | GGCCGUCGAUGUG | CGUUmGmCUAmCAAU* | | |
| | | | CCGCAACGCUCUG | AAGmGmCCmGmUmC(L | | |
| | | | CCGGCAUCGUU | 1)mGmAmUGUGCmCGm | | |
| | | | | CAAmCGCUCUmGmCC( | | |
| | | | | L1)GGCAUCGmU*mU | | |
| G023434 | 495 | CAGAGAAG | CAGAGAAGACAAA | mC*mA*mG*mAmGAAm | 2495, | chr16: |
| | | ACAAAGUC | GUCGUACUGGGGU | GmACmAAmAGUCGmUA | 3029 | 10906819- |
| | | GUACUGGG | UGUAGCUCCCUCC | CUmGGGmGUUGmUmAm | and | 10906843 |
| | | | GUUGCUACAAUAA | GmCUCCCmU(L1)mCm | 3124 | |
| | | | GGCCGUCGAUGUG | CGUUmGmCUAmCAAU* | | |
| | | | CCGCAACGCUCUG | AAGmGmCCmGmUmC(L | | |
| | | | CCGGCAUCGUU | 1)mGmAmUGUGCmCGm | | |
| | | | | CAAmCGCUCUmGmCC( | | |
| | | | | L1)GGCAUCGmU*mU | | |
| G023435 | 496 | AGUACGAC | AGUACGACUUUGU | mA*mG*mU*mAmCGAm | 2496, | chr16: |
| | | UUUGUCUU | CUUCUCUGUCCGU | CmUUmUGmUCUUCmUC | 3030 | 10906822- |
| | | CUCUGUCC | UGUAGCUCCCUCC | UGmUCCmGUUGmUmAm | and | 10906846 |
| | | | GUUGCUACAAUAA | GmCUCCCmU(L1)mCm | 3125 | |
| | | | GGCCGUCGAUGUG | CGUUmGmCUAmCAAU* | | |
| | | | CCGCAACGCUCUG | AAGmGmCCmGmUmC(L | | |
| | | | CCGGCAUCGUU | 1)mGmAmUGUGCmCGm | | |
| | | | | CAAmCGCUCUmGmCC( | | |
| | | | | L1)GGCAUCGmU*mU | | |
| G023436 | 497 | AGCAGAUC | AGCAGAUCCUGCA | mA*mG*mC*mAmGAUm | 2497, | chr16: |
| | | CUGCAGGC | GGCCAUAGGCAGU | CmCUmGCmAGGCCmAU | 3031 | 10906874- |
| | | CAUAGGCA | UGUAGCUCCCUCC | AGmGCAmGUUGmUmAm | and | 10906898 |
| | | | GUUGCUACAAUAA | GmCUCCCmU(L1)mCm | 3126 | |
| | | | GGCCGUCGAUGUG | CGUUmGmCUAmCAAU* | | |
| | | | CCGCAACGCUCUG | AAGmGmCCmGmUmC(L | | |
| | | | CCGGCAUCGUU | 1)mGmAmUGUGCmCGm | | |
| | | | | CAAmCGCUCUmGmCC( | | |
| | | | | L1)GGCAUCGmU*mU | | |
| G023437 | 498 | GCCUAUGG | GCCUAUGGCCUGC | mG*mC*mC*mUmAUGm | 2498, | chr16: |
| | | CCUGCAGG | AGGAUCUGCUCGU | GmCCmUGmCAGGAmUC | 3032 | 10906875- |
| | | AUCUGCUC | UGUAGCUCCCUCC | UGmCUCmGUUGmUmAm | and | 10906899 |
| | | | GUUGCUACAAUAA | GmCUCCCmU(L1)mCm | 3127 | |
| | | | GGCCGUCGAUGUG | CGUUmGmCUAmCAAU* | | |
| | | | CCGCAACGCUCUG | AAGmGmCCmGmUmC(L | | |
| | | | CCGGCAUCGUU | 1)mGmAmUGUGCmCGm | | |
| | | | | CAAmCGCUCUmGmCC( | | |
| | | | | L1)GGCAUCGmU*mU | | |
| G023438 | 499 | GAGCAGAU | GAGCAGAUCCUGC | mG*mA*mG*mCmAGAm | 2499, | chr16: |
| | | CCUGCAGG | AGGCCAUAGGCGU | UmCCmUGmCAGGCmCA | 3033 | 10906875- |
| | | CCAUAGGC | UGUAGCUCCCUCC | UAmGGCmGUUGmUmAm | and | 10906899 |
| | | | GUUGCUACAAUAA | GmCUCCCmU(L1)mCm | 3128 | |
| | | | GGCCGUCGAUGUG | CGUUmGmCUAmCAAU* | | |
| | | | CCGCAACGCUCUG | AAGmGmCCmGmUmC(L | | |
| | | | CCGGCAUCGUU | 1)mGmAmUGUGCmCGm | | |
| | | | | CAAmCGCUCUmGmCC( | | |
| | | | | L1)GGCAUCGmU*mU | | |
| G023439 | 500 | CCUAUGGC | CCUAUGGCCUGCA | mC*mC*mU*mAmUGGm | 2500, | chr16: |
| | | CUGCAGGA | GGAUCUGCUCUGU | CmCUmGCmAGGAUmCU | 3034 | 10906876- |
| | | UCUGCUCU | UGUAGCUCCCUCC | GCmUCUmGUUGmUmAm | and | 10906900 |
| | | | GUUGCUACAAUAA | GmCUCCCmU(L1)mCm | 3129 | |
| | | | GGCCGUCGAUGUG | CGUUmGmCUAmCAAU* | | |
| | | | CCGCAACGCUCUG | AAGmGmCCmGmUmC(L | | |
| | | | CCGGCAUCGUU | 1)mGmAmUGUGCmCGm | | |
| | | | | CAAmCGCUCUmGmCC( | | |
| | | | | L1)GGCAUCGmU*mU | | |
| G023440 | 501 | AGAGCAGA | AGAGCAGAUCCUG | mA*mG*mA*mGmCAGm | 2501, | chr16: |
| | | UCCUGCAG | CAGGCCAUAGGGU | AmUCmCUmGCAGGmCC | 3035 | 10906876- |
| | | GCCAUAGG | UGUAGCUCCCUCC | AUmAGGmGUUGmUmAm | and | 10906900 |
| | | | GUUGCUACAAUAA | GmCUCCCmU(L1)mCm | 3130 | |
| | | | GGCCGUCGAUGUG | CGUUmGmCUAmCAAU* | | |
| | | | CCGCAACGCUCUG | AAGmGmCCmGmUmC(L | | |
| | | | CCGGCAUCGUU | 1)mGmAmUGUGCmCGm | | |
| | | | | CAAmCGCUCUmGmCC( | | |
| | | | | L1)GGCAUCGmU*mU | | |
| G023441 | 502 | AAGAGCAG | AAGAGCAGAUCCU | mA*mA*mG*mAmGCAm | 2502, | chr16: |
| | | AUCCUGCA | GCAGGCCAUAGGU | GmAUmCCmUGCAGmGC | 3036 | 10906877- |
| | | GGCCAUAG | UGUAGCUCCCUCC | CAmUAGmGUUGmUmAm | and | 10906901 |
| | | | GUUGCUACAAUAA | GmCUCCCmU(L1)mCm | 3131 | |
| | | | GGCCGUCGAUGUG | CGUUmGmCUAmCAAU* | | |
| | | | CCGCAACGCUCUG | AAGmGmCCmGmUmC(L | | |
| | | | CCGGCAUCGUU | 1)mGmAmUGUGCmCGm | | |
| | | | | CAAmCGCUCUmGmCC( | | |
| | | | | L1)GGCAUCGmU*mU | | |
| G023442 | 503 | GCCUGCAG | GCCUGCAGGAUCU | mG*mC*mC*mUmGCAm | 2503, | chr16: |
| | | GAUCUGCU | GCUCUUCUCCCGU | GmGAmUCmUGCUCmUU | 3037 | 10906882- |
| | | CUUCUCCC | UGUAGCUCCCUCC | CUmCCCmGUUGmUmAm | and | 10906906 |
| | | | GUUGCUACAAUAA | GmCUCCCmU(L1)mCm | 3132 | |
| | | | GGCCGUCGAUGUG | CGUUmGmCUAmCAAU* | | |
| | | | CCGCAACGCUCUG | AAGmGmCCmGmUmC(L | | |
| | | | CCGGCAUCGUU | 1)mGmAmUGUGCmCGm | | |
| | | | | CAAmCGCUCUmGmCC( | | |
| | | | | L1)GGCAUCGmU*mU | | |
| G023443 | 504 | GGAUCUGC | GGAUCUGCUCUUC | mG*mG*mA*mUmCUGm | 2504, | chr16: |
| | | UCUUCUCC | UCCCUGGGCCCGU | CmUCmUUmCUCCCmUG | 3038 | 10906889- |
| | | CUGGGCCC | UGUAGCUCCCUCC | GGmCCCmGUUGmUmAm | and | 10906913 |
| | | | GUUGCUACAAUAA | GmCUCCCmU(L1)mCm | 3133 | |
| | | | GGCCGUCGAUGUG | CGUUmGmCUAmCAAU* | | |
| | | | CCGCAACGCUCUG | AAGmGmCCmGmUmC(L | | |
| | | | CCGGCAUCGUU | 1)mGmAmUGUGCmCGm | | |
| | | | | CAAmCGCUCUmGmCC( | | |
| | | | | L1)GGCAUCGmU*mU | | |
| G023444 | 505 | CACUCGUG | CACUCGUGGCGGC | mC*mA*mC*mUmCGUm | 2505, | chr16: |
| | | GCGGCCGA | CGAUGAGGUUUGU | GmGCmGGmCCGAUmGA | 3039 | 10906918- |
| | | UGAGGUUU | UGUAGCUCCCUCC | GGmUUUmGUUGmUmAm | and | 10906942 |
| | | | GUUGCUACAAUAA | GmCUCCCmU(L1)mCm | 3134 | |
| | | | GGCCGUCGAUGUG | CGUUmGmCUAmCAAU* | | |
| | | | CCGCAACGCUCUG | AAGmGmCCmGmUmC(L | | |
| | | | CCGGCAUCGUU | 1)mGmAmUGUGCmCGm | | |
| | | | | CAAmCGCUCUmGmCC( | | |
| | | | | L1)GGCAUCGmU*mU | | |
| G023445 | 506 | UGAGGUUU | UGAGGUUUUCAGC | mU*mG*mA*mGmGUUm | 2506, | chr16: |
| | | UCAGCCAC | CACAUCUUGAAGU | UmUCmAGmCCACAmUC | 3040 | 10906934- |
| | | AUCUUGAA | UGUAGCUCCCUCC | UUmGAAmGUUGmUmAm | and | 10906958 |
| | | | GUUGCUACAAUAA | GmCUCCCmU(L1)mCm | 3135 | |
| | | | GGCCGUCGAUGUG | CGUUmGmCUAmCAAU* | | |
| | | | CCGCAACGCUCUG | AAGmGmCCmGmUmC(L | | |
| | | | CCGGCAUCGUU | 1)mGmAmUGUGCmCGm | | |
| | | | | CAAmCGCUCUmGmCC( | | |
| | | | | L1)GGCAUCGmU*mU | | |
| G023446 | 507 | UGAAGAGA | UGAAGAGACCUGA | mU*mG*mA*mAmGAGm | 2507, | chr16: |
| | | CCUGACCG | CCGCGUUCUGCGU | AmCCmUGmACCGCmGU | 3041 | 10906954- |
| | | CGUUCUGC | UGUAGCUCCCUCC | UCmUGCmGUUGmUmAm | and | 10906978 |
| | | | GUUGCUACAAUAA | GmCUCCCmU(L1)mCm | 3136 | |
| | | | GGCCGUCGAUGUG | CGUUmGmCUAmCAAU* | | |
| | | | CCGCAACGCUCUG | AAGmGmCCmGmUmC(L | | |
| | | | CCGGCAUCGUU | 1)mGmAmUGUGCmCGm | | |
| | | | | CAAmCGCUCUmGmCC( | | |
| | | | | L1)GGCAUCGmU*mU | | |
| G023447 | 508 | UCGAGGAG | UCGAGGAGCUGGA | mU*mC*mG*mAmGGAm | 2508, | chr16: |
| | | CUGGAAGC | AGCGCAAGAUGGU | GmCUmGGmAAGCGmCA | 3042 | 10906993- |
| | | GCAAGAUG | UGUAGCUCCCUCC | AGmAUGmGUUGmUmAm | and | 10907017 |
| | | | GUUGCUACAAUAA | GmCUCCCmU(L1)mCm | 3137 | |
| | | | GGCCGUCGAUGUG | CGUUmGmCUAmCAAU* | | |
| | | | CCGCAACGCUCUG | AAGmGmCCmGmUmC(L | | |
| | | | CCGGCAUCGUU | 1)mGmAmUGUGCmCGm | | |
| | | | | CAAmCGCUCUmGmCC( | | |
| | | | | L1)GGCAUCGmU*mU | | |
| G023448 | 509 | AGAUGGCU | AGAUGGCUUCCUG | mA*mG*mA*mUmGGCm | 2509, | chr16: |
| | | UCCUGCAC | CACAGCACGUGGU | UmUCmCUmGCACAmGC | 3043 | 10907012- |
| | | AGCACGUG | UGUAGCUCCCUCC | ACmGUGmGUUGmUmAm | and | 10907036 |
| | | | GUUGCUACAAUAA | GmCUCCCmU(L1)mCm | 3138 | |
| | | | GGCCGUCGAUGUG | CGUUmGmCUAmCAAU* | | |
| | | | CCGCAACGCUCUG | AAGmGmCCmGmUmC(L | | |
| | | | CCGGCAUCGUU | 1)mGmAmUGUGCmCGm | | |
| | | | | CAAmCGCUCUmGmCC( | | |
| | | | | L1)GGCAUCGmU*mU | | |
| G023449 | 510 | UGGCCGGC | UGGCCGGCCUUUU | mU*mG*mG*mCmCGGm | 2510, | chr16: |
| | | CUUUUCCA | CCAGAAGAAGCGU | CmCUmUUmUCCAGmAA | 3044 | 10907077- |
| | | GAAGAAGC | UGUAGCUCCCUCC | GAmAGCmGUUGmUmAm | and | 10907101 |
| | | | GUUGCUACAAUAA | GmCUCCCmU(L1)mCm | 3139 | |
| | | | GGCCGUCGAUGUG | CGUUmGmCUAmCAAU* | | |
| | | | CCGCAACGCUCUG | AAGmGmCCmGmUmC(L | | |
| | | | CCGGCAUCGUU | 1)mGmAmUGUGCmCGm | | |
| | | | | CAAmCGCUCUmGmCC( | | |
| | | | | L1)GGCAUCGmU*mU | | |
| G023450 | 511 | UUCCAGAA | UUCCAGAAGAAGC | mU*mU*mC*mCmAGAm | 2511, | chr16: |
| | | GAAGCUGC | UGCUCCGAGGUGU | AmGAmAGmCUGCUmCC | 3045 | 10907088- |
| | | UCCGAGGU | UGUAGCUCCCUCC | GAmGGUmGUUGmUmAm | and | 10907112 |
| | | | GUUGCUACAAUAA | GmCUCCCmU(L1)mCm | 3140 | |
| | | | GGCCGUCGAUGUG | CGUUmGmCUAmCAAU* | | |
| | | | CCGCAACGCUCUG | AAGmGmCCmGmUmC(L | | |
| | | | CCGGCAUCGUU | 1)mGmAmUGUGCmCGm | | |
| | | | | CAAmCGCUCUmGmCC( | | |
| | | | | L1)GGCAUCGmU*mU | | |
| G023451 | 512 | UCCAGAAG | UCCAGAAGAAGCU | mU*mC*mC*mAmGAAm | 2512, | chr16: |
| | | AAGCUGCU | GCUCCGAGGUUGU | GmAAmGCmUGCUCmCG | 3046 | 10907089- |
| | | CCGAGGUU | UGUAGCUCCCUCC | AGmGUUmGUUGmUmAm | and | 10907113 |
| | | | GUUGCUACAAUAA | GmCUCCCmU(L1)mCm | 3141 | |
| | | | GGCCGUCGAUGUG | CGUUmGmCUAmCAAU* | | |
| | | | CCGCAACGCUCUG | AAGmGmCCmGmUmC(L | | |
| | | | CCGGCAUCGUU | 1)mGmAmUGUGCmCGm | | |
| | | | | CAAmCGCUCUmGmCC( | | |
| | | | | L1)GGCAUCGmU*mU | | |
| G023452 | 513 | AGAAGAAG | AGAAGAAGCUGCU | mA*mG*mA*mAmGAAm | 2513, | chr16: |
| | | CUGCUCCG | CCGAGGUUGCAGU | GmCUmGCmUCCGAmGG | 3047 | 10907092- |
| | | AGGUUGCA | UGUAGCUCCCUCC | UUmGCAmGUUGmUmAm | and | 10907116 |
| | | | GUUGCUACAAUAA | GmCUCCCmU(L1)mCm | 3142 | |
| | | | GGCCGUCGAUGUG | CGUUmGmCUAmCAAU* | | |
| | | | CCGCAACGCUCUG | AAGmGmCCmGmUmC(L | | |
| | | | CCGGCAUCGUU | 1)mGmAmUGUGCmCGm | | |
| | | | | CAAmCGCUCUmGmCC( | | |
| | | | | L1)GGCAUCGmU*mU | | |
| G023453 | 514 | AGAAGCUG | AGAAGCUGCUCCG | mA*mG*mA*mAmGCUm | 2514, | chr16: |
| | | CUCCGAGG | AGGUUGCACCCGU | GmCUmCCmGAGGUmUG | 3048 | 10907095- |
| | | UUGCACCC | UGUAGCUCCCUCC | CAmCCCmGUUGmUmAm | and | 10907119 |
| | | | GUUGCUACAAUAA | GmCUCCCmU(L1)mCm | 3143 | |
| | | | GGCCGUCGAUGUG | CGUUmGmCUAmCAAU* | | |
| | | | CCGCAACGCUCUG | AAGmGmCCmGmUmC(L | | |
| | | | CCGGCAUCGUU | 1)mGmAmUGUGCmCGm | | |
| | | | | CAAmCGCUCUmGmCC( | | |
| | | | | L1)GGCAUCGmU*mU | | |
| G023454 | 515 | CUCCGAGG | CUCCGAGGUUGCA | mC*mU*mC*mCmGAGm | 2515, | chr16: |
| | | UUGCACCC | CCCUCCUCCUCGU | GmUUmGCmACCCUmCC | 3049 | 10907103- |
| | | UCCUCCUC | UGUAGCUCCCUCC | UCmCUCmGUUGmUmAm | and | 10907127 |
| | | | GUUGCUACAAUAA | GmCUCCCmU(L1)mCm | 3144 | |
| | | | GGCCGUCGAUGUG | CGUUmGmCUAmCAAU* | | |
| | | | CCGCAACGCUCUG | AAGmGmCCmGmUmC(L | | |
| | | | CCGGCAUCGUU | 1)mGmAmUGUGCmCGm | | |
| | | | | CAAmCGCUCUmGmCC( | | |
| | | | | L1)GGCAUCGmU*mU | | |
| G023455 | 516 | AGGUUGCA | AGGUUGCACCCUC | mA*mG*mG*mUmUGCm | 2516, | chr16: |
| | | CCCUCCUC | CUCCUCACAGCGU | AmCCmCUmCCUCCmUC | 3050 | 10907108- |
| | | CUCACAGC | UGUAGCUCCCUCC | ACmAGCmGUUGmUmAm | and | 10907132 |
| | | | GUUGCUACAAUAA | GmCUCCCmU(L1)mCm | 3145 | |
| | | | GGCCGUCGAUGUG | CGUUmGmCUAmCAAU* | | |
| | | | CCGCAACGCUCUG | AAGmGmCCmGmUmC(L | | |
| | | | CCGGCAUCGUU | 1)mGmAmUGUGCmCGm | | |
| | | | | CAAmCGCUCUmGmCC( | | |
| | | | | L1)GGCAUCGmU*mU | | |
| G023456 | 517 | CUGGUCCA | CUGGUCCAGAGCC | mC*mU*mG*mGmUCCm | 2517, | chr16: |
| | | GAGCCUGA | UGAGCAAGGCCGU | AmGAmGCmCUGAGmCA | 3051 | 10907148- |
| | | GCAAGGCC | UGUAGCUCCCUCC | AGmGCCmGUUGmUmAm | and | 10907172 |
| | | | GUUGCUACAAUAA | GmCUCCCmU(L1)mCm | 3146 | |
| | | | GGCCGUCGAUGUG | CGUUmGmCUAmCAAU* | | |
| | | | CCGCAACGCUCUG | AAGmGmCCmGmUmC(L | | |
| | | | CCGGCAUCGUU | 1)mGmAmUGUGCmCGm | | |
| | | | | CAAmCGCUCUmGmCC( | | |
| | | | | L1)GGCAUCGmU*mU | | |
| G023457 | 518 | UGGUCCAG | UGGUCCAGAGCCU | mU*mG*mG*mUmCCAm | 2518, | chr16: |
| | | AGCCUGAG | GAGCAAGGCCGGU | GmAGmCCmUGAGCmAA | 3052 | 10907149- |
| | | CAAGGCCG | UGUAGCUCCCUCC | GGmCCGmGUUGmUmAm | and | 10907173 |
| | | | GUUGCUACAAUAA | GmCUCCCmU(L1)mCm | 3147 | |
| | | | GGCCGUCGAUGUG | CGUUmGmCUAmCAAU* | | |
| | | | CCGCAACGCUCUG | AAGmGmCCmGmUmC(L | | |
| | | | CCGGCAUCGUU | 1)mGmAmUGUGCmCGm | | |
| | | | | CAAmCGCUCUmGmCC( | | |
| | | | | L1)GGCAUCGmU*mU | | |
| G023458 | 519 | AGCAAGGC | AGCAAGGCCGACG | mA*mG*mC*mAmAGGm | 2519, | chr16: |
| | | CGACGCCC | CCCUAUUUGAGGU | CmCGmACmGCCCUmAU | 3053 | 10907163- |
| | | UAUUUGAG | UGUAGCUCCCUCC | UUmGAGmGUUGmUmAm | and | 10907187 |
| | | | GUUGCUACAAUAA | GmCUCCCmU(L1)mCm | 3148 | |
| | | | GGCCGUCGAUGUG | CGUUmGmCUAmCAAU* | | |
| | | | CCGCAACGCUCUG | AAGmGmCCmGmUmC(L | | |
| | | | CCGGCAUCGUU | 1)mGmAmUGUGCmCGm | | |
| | | | | CAAmCGCUCUmGmCC( | | |
| | | | | L1)GGCAUCGmU*mU | | |
| G023459 | 520 | GACGCCCU | GACGCCCUAUUUG | mG*mA*mC*mGmCCCm | 2520, | chr16: |
| | | AUUUGAGC | AGCUGUCCGGCGU | UmAUmUUmGAGCUmGU | 3054 | 10907172- |
| | | UGUCCGGC | UGUAGCUCCCUCC | CCmGGCmGUUGmUmAm | and | 10907196 |
| | | | GUUGCUACAAUAA | GmCUCCCmU(L1)mCm | 3149 | |
| | | | GGCCGUCGAUGUG | CGUUmGmCUAmCAAU* | | |
| | | | CCGCAACGCUCUG | AAGmGmCCmGmUmC(L | | |
| | | | CCGGCAUCGUU | 1)mGmAmUGUGCmCGm | | |
| | | | | CAAmCGCUCUmGmCC( | | |
| | | | | L1)GGCAUCGmU*mU | | |
| G023460 | 521 | AAGCCGGA | AAGCCGGACAGCU | mA*mA*mG*mCmCGGm | 2521, | chr16: |
| | | CAGCUCAA | CAAAUAGGGCGGU | AmCAmGCmUCAAAmUA | 3055 | 10907174- |
| | | AUAGGGCG | UGUAGCUCCCUCC | GGmGCGmGUUGmUmAm | and | 10907198 |
| | | | GUUGCUACAAUAA | GmCUCCCmU(L1)mCm | 3150 | |
| | | | GGCCGUCGAUGUG | CGUUmGmCUAmCAAU* | | |
| | | | CCGCAACGCUCUG | AAGmGmCCmGmUmC(L | | |
| | | | CCGGCAUCGUU | 1)mGmAmUGUGCmCGm | | |
| | | | | CAAmCGCUCUmGmCC( | | |
| | | | | L1)GGCAUCGmU*mU | | |
| G023461 | 522 | GAGCUGUC | GAGCUGUCCGGCU | mG*mA*mG*mCmUGUm | 2522, | chr16: |
| | | CGGCUUCU | UCUCCAUGGAGGU | CmCGmGCmUUCUCmCA | 3056 | 10907184- |
| | | CCAUGGAG | UGUAGCUCCCUCC | UGmGAGmGUUGmUmAm | and | 10907208 |
| | | | GUUGCUACAAUAA | GmCUCCCmU(L1)mCm | 3151 | |
| | | | GGCCGUCGAUGUG | CGUUmGmCUAmCAAU* | | |
| | | | CCGCAACGCUCUG | AAGmGmCCmGmUmC(L | | |
| | | | CCGGCAUCGUU | 1)mGmAmUGUGCmCGm | | |
| | | | | CAAmCGCUCUmGmCC( | | |
| | | | | L1)GGCAUCGmU*mU | | |
| G023462 | 523 | AGCUGUCC | AGCUGUCCGGCUU | mA*mG*mC*mUmGUCm | 2523, | chr16: |
| | | GGCUUCUC | CUCCAUGGAGCGU | CmGGmCUmUCUCCmAU | 3057 | 10907185- |
| | | CAUGGAGC | UGUAGCUCCCUCC | GGmAGCmGUUGmUmAm | and | 10907209 |
| | | | GUUGCUACAAUAA | GmCUCCCmU(L1)mCm | 3152 | |
| | | | GGCCGUCGAUGUG | CGUUmGmCUAmCAAU* | | |
| | | | CCGCAACGCUCUG | AAGmGmCCmGmUmC(L | | |
| | | | CCGGCAUCGUU | 1)mGmAmUGUGCmCGm | | |
| | | | | CAAmCGCUCUmGmCC( | | |
| | | | | L1)GGCAUCGmU*mU | | |
| G023463 | 524 | UCAGGGAU | UCAGGGAUGACAG | mU*mC*mA*mGmGGAm | 2524, | chr16: |
| | | GACAGAGC | AGCACCAAGACGU | UmGAmCAmGAGCAmCC | 3058 | 10907244- |
| | | ACCAAGAC | UGUAGCUCCCUCC | AAmGACmGUUGmUmAm | and | 10907268 |
| | | | GUUGCUACAAUAA | GmCUCCCmU(L1)mCm | 3153 | |
| | | | GGCCGUCGAUGUG | CGUUmGmCUAmCAAU* | | |
| | | | CCGCAACGCUCUG | AAGmGmCCmGmUmC(L | | |
| | | | CCGGCAUCGUU | 1)mGmAmUGUGCmCGm | | |
| | | | | CAAmCGCUCUmGmCC( | | |
| | | | | L1)GGCAUCGmU*mU | | |
| G023464 | 525 | CAGAGCAC | CAGAGCACCAAGA | mC*mA*mG*mAmGCAm | 2525, | chr16: |
| | | CAAGACAG | CAGAGCCCUGAGU | CmCAmAGmACAGAmGC | 3059 | 10907254- |
| | | AGCCCUGA | UGUAGCUCCCUCC | CCmUGAmGUUGmUmAm | and | 10907278 |
| | | | GUUGCUACAAUAA | GmCUCCCmU(L1)mCm | 3154 | |
| | | | GGCCGUCGAUGUG | CGUUmGmCUAmCAAU* | | |
| | | | CCGCAACGCUCUG | AAGmGmCCmGmUmC(L | | |
| | | | CCGGCAUCGUU | 1)mGmAmUGUGCmCGm | | |
| | | | | CAAmCGCUCUmGmCC( | | |
| | | | | L1)GGCAUCGmU*mU | | |
| G023465 | 526 | AGCACCAA | AGCACCAAGACAG | mA*mG*mC*mAmCCAm | 2526, | chr16: |
| | | GACAGAGC | AGCCCUGACGCGU | AmGAmCAmGAGCCmCU | 3060 | 10907257- |
| | | CCUGACGC | UGUAGCUCCCUCC | GAmCGCmGUUGmUmAm | and | 10907281 |
| | | | GUUGCUACAAUAA | GmCUCCCmU(L1)mCm | 3155 | |
| | | | GGCCGUCGAUGUG | CGUUmGmCUAmCAAU* | | |
| | | | CCGCAACGCUCUG | AAGmGmCCmGmUmC(L | | |
| | | | CCGGCAUCGUU | 1)mGmAmUGUGCmCGm | | |
| | | | | CAAmCGCUCUmGmCC( | | |
| | | | | L1)GGCAUCGmU*mU | | |
| G023466 | 527 | GGGACCGG | GGGACCGGCCACU | mG*mG*mG*mAmCCGm | 2527, | chr16: |
| | | CCACUUCU | UCUUCUCAGUCGU | GmCCmACmUUCUUmCU | 3061 | 10907287- |
| | | UCUCAGUC | UGUAGCUCCCUCC | CAmGUCmGUUGmUmAm | and | 10907311 |
| | | | GUUGCUACAAUAA | GmCUCCCmU(L1)mCm | 3156 | |
| | | | GGCCGUCGAUGUG | CGUUmGmCUAmCAAU* | | |
| | | | CCGCAACGCUCUG | AAGmGmCCmGmUmC(L | | |
| | | | CCGGCAUCGUU | 1)mGmAmUGUGCmCGm | | |
| | | | | CAAmCGCUCUmGmCC( | | |
| | | | | L1)GGCAUCGmU*mU | | |
| G023467 | 528 | GGCCACUU | GGCCACUUCUUCU | mG*mG*mC*mCmACUm | 2528 | chr16: |
| | | CUUCUCAG | CAGUCACAGCCGU | UmCUmUCmUCAGUmCA | 3062 | 10907293- |
| | | UCACAGCC | UGUAGCUCCCUCC | CAmGCCmGUUGmUmAm | and | 10907317 |
| | | | GUUGCUACAAUAA | GmCUCCCmU(L1)mCm | 3157 | |
| | | | GGCCGUCGAUGUG | CGUUmGmCUAmCAAU* | | |
| | | | CCGCAACGCUCUG | AAGmGmCCmGmUmC(L | | |
| | | | CCGGCAUCGUU | 1)mGmAmUGUGCmCGm | | |
| | | | | CAAmCGCUCUmGmCC( | | |
| | | | | L1)GGCAUCGmU*mU | | |
| G023468 | 529 | ACAGCCCU | ACAGCCCUACUUU | mA*mC*mA*mGmCCCm | 2529 | chr16: |
| | | ACUUUGUG | GUGCCGGGCAGGU | UmACmUUmUGUGCmCG | 3063 | 10907317- |
| | | CCGGGCAG | UGUAGCUCCCUCC | GGmCAGmGUUGmUmAm | and | 10907341 |
| | | | GUUGCUACAAUAA | GmCUCCCmU(L1)mCm | 3158 | |
| | | | GGCCGUCGAUGUG | CGUUmGmCUAmCAAU* | | |
| | | | CCGCAACGCUCUG | AAGmGmCCmGmUmC(L | | |
| | | | CCGGCAUCGUU | 1)mGmAmUGUGCmCGm | | |
| | | | | CAAmCGCUCUmGmCC( | | |
| | | | | L1)GGCAUCGmU*mU | | |
| G023469 | 530 | UGCCGGGC | UGCCGGGCAGUGU | mU*mG*mC*mCmGGGm | 2530, | chr16: |
| | | AGUGUGCC | GCCAGCUCUCAGU | CmAGmUGmUGCCAmGC | 3064 | 10907331- |
| | | AGCUCUCA | UGUAGCUCCCUCC | UCmUCAmGUUGmUmAm | and | 10907355 |
| | | | GUUGCUACAAUAA | GmCUCCCmU(L1)mCm | 3159 | |
| | | | GGCCGUCGAUGUG | CGUUmGmCUAmCAAU* | | |
| | | | CCGCAACGCUCUG | AAGmGmCCmGmUmC(L | | |
| | | | CCGGCAUCGUU | 1)mGmAmUGUGCmCGm | | |
| | | | | CAAmCGCUCUmGmCC( | | |
| | | | | L1)GGCAUCGmU*mU | | |
| G023470 | 531 | GCCGGGCA | GCCGGGCAGUGUG | mG*mC*mC*mGmGGCm | 2531, | chr16: |
| | | GUGUGCCA | CCAGCUCUCAGGU | AmGUmGUmGCCAGmCU | 3065 | 10907332- |
| | | GCUCUCAG | UGUAGCUCCCUCC | CUmCAGmGUUGmUmAm | and | 10907356 |
| | | | GUUGCUACAAUAA | GmCUCCCmU(L1)mCm | 3160 | |
| | | | GGCCGUCGAUGUG | CGUUmGmCUAmCAAU* | | |
| | | | CCGCAACGCUCUG | AAGmGmCCmGmUmC(L | | |
| | | | CCGGCAUCGUU | 1)mGmAmUGUGCmCGm | | |
| | | | | CAAmCGCUCUmGmCC( | | |
| | | | | L1)GGCAUCGmU*mU | | |
| G023471 | 532 | CCUCCACG | CCUCCACGCUCAC | mC*mC*mU*mCmCACm | 2532, | chr16: |
| | | CUCACGGG | GGGACUCUAUGGU | GmCUmCAmCGGGAmCU | 3066 | 10907392- |
| | | ACUCUAUG | UGUAGCUCCCUCC | CUmAUGmGUUGmUmAm | and | 10907416 |
| | | | GUUGCUACAAUAA | GmCUCCCmU(L1)mCm | 3161 | |
| | | | GGCCGUCGAUGUG | CGUUmGmCUAmCAAU* | | |
| | | | CCGCAACGCUCUG | AAGmGmCCmGmUmC(L | | |
| | | | CCGGCAUCGUU | 1)mGmAmUGUGCmCGm | | |
| | | | | CAAmCGCUCUmGmCC( | | |
| | | | | L1)GGCAUCGmU*mU | | |
| G023472 | 533 | UCACGGGA | UCACGGGACUCUA | mU*mC*mA*mCmGGGm | 2533, | chr16: |
| | | CUCUAUGU | UGUCGGCCUGCGU | AmCUmCUmAUGUCmGG | 3067 | 10907401- |
| | | CGGCCUGC | UGUAGCUCCCUCC | CCmUGCmGUUGmUmAm | and | 10907425 |
| | | | GUUGCUACAAUAA | GmCUCCCmU(L1)mCm | 3162 | |
| | | | GGCCGUCGAUGUG | CGUUmGmCUAmCAAU* | | |
| | | | CCGCAACGCUCUG | AAGmGmCCmGmUmC(L | | |
| | | | CCGGCAUCGUU | 1)mGmAmUGUGCmCGm | | |
| | | | | CAAmCGCUCUmGmCC( | | |
| | | | | L1)GGCAUCGmU*mU | | |
| G023473 | 534 | UGGGAGCU | UGGGAGCUGGGCC | mU*mG*mG*mGmAGCm | 2534, | chr16: |
| | | GGGCCGCA | GCAGACAUCAAGU | UmGGmGCmCGCAGmAC | 3068 | 10907484- |
| | | GACAUCAA | UGUAGCUCCCUCC | AUmCAAmGUUGmUmAm | and | 10907508 |
| | | | GUUGCUACAAUAA | GmCUCCCmU(L1)mCm | 3163 | |
| | | | GGCCGUCGAUGUG | CGUUmGmCUAmCAAU* | | |
| | | | CCGCAACGCUCUG | AAGmGmCCmGmUmC(L | | |
| | | | CCGGCAUCGUU | 1)mGmAmUGUGCmCGm | | |
| | | | | CAAmCGCUCUmGmCC( | | |
| | | | | L1)GGCAUCGmU*mU | | |
| G023474 | 535 | GGGAGCUG | GGGAGCUGGGCCG | mG*mG*mG*mAmGCUm | 2535, | chr16: |
| | | GGCCGCAG | CAGACAUCAAAGU | GmGGmCCmGCAGAmCA | 3069 | 91007485- |
| | | ACAUCAAA | UGUAGCUCCCUCC | UCmAAAmGUUGmUmAm | and | 10907509 |
| | | | GUUGCUACAAUAA | GmCUCCCmU(L1)mCm | 3164 | |
| | | | GGCCGUCGAUGUG | CGUUmGmCUAmCAAU* | | |
| | | | CCGCAACGCUCUG | AAGmGmCCmGmUmC(L | | |
| | | | CCGGCAUCGUU | 1)mGmAmUGUGCmCGm | | |
| | | | | CAAmCGCUCUmGmCC( | | |
| | | | | L1)GGCAUCGmU*mU | | |
| G023475 | 536 | GCAGACAU | GCAGACAUCAAAG | mG*mC*mA*mGmACAm | 2536, | chr16: |
| | | CAAAGUAC | UACCCUACAGGGU | UmCAmAAmGUACCmCU | 3070 | 10907497- |
| | | CCUACAGG | UGUAGCUCCCUCC | ACmAGGmGUUGmUmAm | and | 10907521 |
| | | | GUUGCUACAAUAA | GmCUCCCmU(L1)mCm | 3165 | |
| | | | GGCCGUCGAUGUG | CGUUmGmCUAmCAAU* | | |
| | | | CCGCAACGCUCUG | AAGmGmCCmGmUmC(L | | |
| | | | CCGGCAUCGUU | 1)mGmAmUGUGCmCGm | | |
| | | | | CAAmCGCUCUmGmCC( | | |
| | | | | L1)GGCAUCGmU*mU | | |
| G023476 | 537 | AUCAAAGU | AUCAAAGUACCCU | mA*mU*mC*mAmAAGm | 2537, | chr16: |
| | | ACCCUACA | ACAGGAGGACCGU | UmACmCCmUACAGmGA | 3071 | 10907503- |
| | | GGAGGACC | UGUAGCUCCCUCC | GGmACCmGUUGmUmAm | and | 10907527 |
| | | | GUUGCUACAAUAA | GmCUCCCmU(L1)mCm | 3166 | |
| | | | GGCCGUCGAUGUG | CGUUmGmCUAmCAAU* | | |
| | | | CCGCAACGCUCUG | AAGmGmCCmGmUmC(L | | |
| | | | CCGGCAUCGUU | 1)mGmAmUGUGCmCGm | | |
| | | | | CAAmCGCUCUmGmCC( | | |
| | | | | L1)GGCAUCGmU*mU | | |
| G023477 | 538 | UCAAAGUA | UCAAAGUACCCUA | mU*mC*mA*mAmAGUm | 2538, | chr16: |
| | | CCCUACAG | CAGGAGGACCAGU | AmCCmCUmACAGGmAG | 3072 | 10907504- |
| | | GAGGACCA | UGUAGCUCCCUCC | GAmCCAmGUUGmUmAm | and | 10907528 |
| | | | GUUGCUACAAUAA | GmCUCCCmU(L1)mCm | 3167 | |
| | | | GGCCGUCGAUGUG | CGUUmGmCUAmCAAU* | | |
| | | | CCGCAACGCUCUG | AAGmGmCCmGmUmC(L | | |
| | | | CCGGCAUCGUU | 1)mGmAmUGUGCmCGm | | |
| | | | | CAAmCGCUCUmGmCC( | | |
| | | | | L1)GGCAUCGmU*mU | | |
| G023478 | 539 | AGUACCCU | AGUACCCUACAGG | mA*mG*mU*mAmCCCm | 2539, | chr16: |
| | | ACAGGAGG | AGGACCAGUUCGU | UmACmAGmGAGGAmCC | 3073 | 10907508- |
| | | ACCAGUUC | UGUAGCUCCCUCC | AGmUUCmGUUGmUmAm | and | 10907532 |
| | | | GUUGCUACAAUAA | GmCUCCCmU(L1)mCm | 3168 | |
| | | | GGCCGUCGAUGUG | CGUUmGmCUAmCAAU* | | |
| | | | CCGCAACGCUCUG | AAGmGmCCmGmUmC(L | | |
| | | | CCGGCAUCGUU | 1)mGmAmUGUGCmCGm | | |
| | | | | CAAmCGCUCUmGmCC( | | |
| | | | | L1)GGCAUCGmU*mU | | |
| G023479 | 540 | UCCGCAGA | UCCGCAGACGUGA | mU*mC*mC*mGmCAGm | 2540, | chr16: |
| | | CGUGAGGA | GGACCUGGGCGGU | AmCGmUGmAGGACmCU | 3074 | 10907535- |
| | | CCUGGGCG | UGUAGCUCCCUCC | GGmGCGmGUUGmUmAm | and | 10907559 |
| | | | GUUGCUACAAUAA | GmCUCCCmU(L1)mCm | 3169 | |
| | | | GGCCGUCGAUGUG | CGUUmGmCUAmCAAU* | | |
| | | | CCGCAACGCUCUG | AAGmGmCCmGmUmC(L | | |
| | | | CCGGCAUCGUU | 1)mGmAmUGUGCmCGm | | |
| | | | | CAAmCGCUCUmGmCC( | | |
| | | | | L1)GGCAUCGmU*mU | | |
| G023480 | 541 | GGACCUGG | GGACCUGGGCGAU | mG*mG*mA*mCmCUGm | 2541, | chr16: |
| | | GCGAUGGC | GGCCAAAGGCUGU | GmGCmGAmUGGCCmAA | 3075 | 10907548- |
| | | CAAAGGCU | UGUAGCUCCCUCC | AGmGCUmGUUGmUmAm | and | 10907572 |
| | | | GUUGCUACAAUAA | GmCUCCCmU(L1)mCm | 3170 | |
| | | | GGCCGUCGAUGUG | CGUUmGmCUAmCAAU* | | |
| | | | CCGCAACGCUCUG | AAGmGmCCmGmUmC(L | | |
| | | | CCGGCAUCGUU | 1)mGmAmUGUGCmCGm | | |
| | | | | CAAmCGCUCUmGmCC( | | |
| | | | | L1)GGCAUCGmU*mU | | |
| G023481 | 542 | GGGCGAUG | GGGCGAUGGCCAA | mG*mG*mG*mCmGAUm | 2542, | chr16: |
| | | GCCAAAGG | AGGCUUAGUCCGU | GmGCmCAmAAGGCmUU | 3076 | 10907554- |
| | | CUUAGUCC | UGUAGCUCCCUCC | AGmUCCmGUUGmUmAm | and | 10907578 |
| | | | GUUGCUACAAUAA | GmCUCCCmU(L1)mCm | 3171 | |
| | | | GGCCGUCGAUGUG | CGUUmGmCUAmCAAU* | | |
| | | | CCGCAACGCUCUG | AAGmGmCCmGmUmC(L | | |
| | | | CCGGCAUCGUU | 1)mGmAmUGUGCmCGm | | |
| | | | | CAAmCGCUCUmGmCC( | | |
| | | | | L1)GGCAUCGmU*mU | | |
| G023482 | 543 | GGCGAUGG | GGCGAUGGCCAAA | mG*mG*mC*mGmAUGm | 2543, | chr16: |
| | | CCAAAGGC | GGCUUAGUCCAGU | GmCCmAAmAGGCUmUA | 3077 | 10907555- |
| | | UUAGUCCA | UGUAGCUCCCUCC | GUmCCAmGUUGmUmAm | and | 10907579 |
| | | | GUUGCUACAAUAA | GmCUCCCmU(L1)mCm | 3172 | |
| | | | GGCCGUCGAUGUG | CGUUmGmCUAmCAAU* | | |
| | | | CCGCAACGCUCUG | AAGmGmCCmGmUmC(L | | |
| | | | CCGGCAUCGUU | 1)mGmAmUGUGCmCGm | | |
| | | | | CAAmCGCUCUmGmCC( | | |
| | | | | L1)GGCAUCGmU*mU | | |
| G023483 | 544 | CGCGGGCC | CGCGGGCCGCAGA | mC*mG*mC*mGmGGCm | 2544, | chr16: |
| | | GCAGAGUC | GUCCGAGCUGGGU | CmGCmAGmAGUCCmGA | 3078 | 10907587- |
| | | CGAGCUGG | UGUAGCUCCCUCC | GCmUGGmGUUGmUmAm | and | 10907611 |
| | | | GUUGCUACAAUAA | GmCUCCCmU(L1)mCm | 3173 | |
| | | | GGCCGUCGAUGUG | CGUUmGmCUAmCAAU* | | |
| | | | CCGCAACGCUCUG | AAGmGmCCmGmUmC(L | | |
| | | | CCGGCAUCGUU | 1)mGmAmUGUGCmCGm | | |
| | | | | CAAmCGCUCUmGmCC( | | |
| | | | | L1)GGCAUCGmU*mU | | |
| G023484 | 545 | GCGGGCCG | GCGGGCCGCAGAG | mG*mC*mG*mGmGCCm | 2545, | chr16: |
| | | CAGAGUCC | UCCGAGCUGGCGU | GmCAmGAmGUCCGmAG | 3079 | 10907588- |
| | | GAGCUGGC | UGUAGCUCCCUCC | CUmGGCmGUUGmUmAm | and | 10907612 |
| | | | GUUGCUACAAUAA | GmCUCCCmU(L1)mCm | 3174 | |
| | | | GGCCGUCGAUGUG | CGUUmGmCUAmCAAU* | | |
| | | | CCGCAACGCUCUG | AAGmGmCCmGmUmC(L | | |
| | | | CCGGCAUCGUU | 1)mGmAmUGUGCmCGm | | |
| | | | | CAAmCGCUCUmGmCC( | | |
| | | | | L1)GGCAUCGmU*mU | | |
| G023485 | 546 | CGGGCCGC | CGGGCCGCAGAGU | mC*mG*mG*mGmCCGm | 2546, | chr16: |
| | | AGAGUCCG | CCGAGCUGGCCGU | CmAGmAGmUCCGAmGC | 3080 | 10907589- |
| | | AGCUGGCC | UGUAGCUCCCUCC | UGmGCCmGUUGmUmAm | and | 10907613 |
| | | | GUUGCUACAAUAA | GmCUCCCmU(L1)mCm | 3175 | |
| | | | GGCCGUCGAUGUG | CGUUmGmCUAmCAAU* | | |
| | | | CCGCAACGCUCUG | AAGmGmCCmGmUmC(L | | |
| | | | CCGGCAUCGUU | 1)mGmAmUGUGCmCGm | | |
| | | | | CAAmCGCUCUmGmCC( | | |
| | | | | L1)GGCAUCGmU*mU | | |
| G023486 | 547 | UGAGUGGC | UGAGUGGCGAAAU | mU*mG*mA*mGmUGGm | 2547, | chr16: |
| | | GAAAUCAA | CAAGGACAAGGGU | CmGAmAAmUCAAGmGA | 3081 | 10907662- |
| | | GGACAAGG | UGUAGCUCCCUCC | CAmAGGmGUUGmUmAm | and | 10907686 |
| | | | GUUGCUACAAUAA | GmCUCCCmU(L1)mCm | 3176 | |
| | | | GGCCGUCGAUGUG | CGUUmGmCUAmCAAU* | | |
| | | | CCGCAACGCUCUG | AAGmGmCCmGmUmC(L | | |
| | | | CCGGCAUCGUU | 1)mGmAmUGUGCmCGm | | |
| | | | | CAAmCGCUCUmGmCC( | | |
| | | | | L1)GGCAUCGmU*mU | | |
| G023487 | 548 | GAGUGGCG | GAGUGGCGAAAUC | mG*mA*mG*mUmGGCm | 2548, | chr16: |
| | | AAAUCAAG | AAGGACAAGGAGU | GmAAmAUmCAAGGmAC | 3082 | 10907663- |
| | | GACAAGGA | UGUAGCUCCCUCC | AAmGGAmGUUGmUmAm | and | 10907687 |
| | | | GUUGCUACAAUAA | GmCUCCCmU(L1)mCm | 3177 | |
| | | | GGCCGUCGAUGUG | CGUUmGmCUAmCAAU* | | |
| | | | CCGCAACGCUCUG | AAGmGmCCmGmUmC(L | | |
| | | | CCGGCAUCGUU | 1)mGmAmUGUGCmCGm | | |
| | | | | CAAmCGCUCUmGmCC( | | |
| | | | | L1)GGCAUCGmU*mU | | |
| G023488 | 549 | AAAUCAAG | AAAUCAAGGACAA | mA*mA*mA*mUmCAAm | 2549, | chr16: |
| | | GACAAGGA | GGAGCUCCCGCGU | GmGAmCAmAGGAGmCU | 3083 | 10907671- |
| | | GCUCCCGC | UGUAGCUCCCUCC | CCmCGCmGUUGmUmAm | and | 10907695 |
| | | | GUUGCUACAAUAA | GmCUCCCmU(L1)mCm | 3178 | |
| | | | GGCCGUCGAUGUG | CGUUmGmCUAmCAAU* | | |
| | | | CCGCAACGCUCUG | AAGmGmCCmGmUmC(L | | |
| | | | CCGGCAUCGUU | 1)mGmAmUGUGCmCGm | | |
| | | | | CAAmCGCUCUmGmCC( | | |
| | | | | L1)GGCAUCGmU*mU | | |
| G023489 | 550 | AAGGAGCU | AAGGAGCUCCCGC | mA*mA*mG*mGmAGCm | 2550, | chr16: |
| | | CCCGCAGU | AGUACCUAGCAGU | UmCCmCGmCAGUAmCC | 3084 | 10907682- |
| | | ACCUAGCA | UGUAGCUCCCUCC | UAmGCAmGUUGmUmAm | and | 10907706 |
| | | | GUUGCUACAAUAA | GmCUCCCmU(L1)mCm | 3179 | |
| | | | GGCCGUCGAUGUG | CGUUmGmCUAmCAAU* | | |
| | | | CCGCAACGCUCUG | AAGmGmCCmGmUmC(L | | |
| | | | CCGGCAUCGUU | 1)mGmAmUGUGCmCGm | | |
| | | | | CAAmCGCUCUmGmCC( | | |
| | | | | L1)GGCAUCGmU*mU | | |
| G023490 | 551 | AGGAGCUC | AGGAGCUCCCGCA | mA*mG*mG*mAmGCUm | 2551, | chr16: |
| | | CCGCAGUA | GUACCUAGCAUGU | CmCCmGCmAGUACmCU | 3085 | 10907683- |
| | | CCUAGCAU | UGUAGCUCCCUCC | AGmCAUmGUUGmUmAm | and | 10907707 |
| | | | GUUGCUACAAUAA | GmCUCCCmU(L1)mCm | 3180 | |
| | | | GGCCGUCGAUGUG | CGUUmGmCUAmCAAU* | | |
| | | | CCGCAACGCUCUG | AAGmGmCCmGmUmC(L | | |
| | | | CCGGCAUCGUU | 1)mGmAmUGUGCmCGm | | |
| | | | | CAAmCGCUCUmGmCC( | | |
| | | | | L1)GGCAUCGmU*mU | | |
| G023491 | 552 | GGAGCUCC | GGAGCUCCCGCAG | mG*mG*mA*mGmCUCm | 2552, | chr16: |
| | | CGCAGUAC | UACCUAGCAUUGU | CmCGmCAmGUACCmUA | 3086 | 10907684- |
| | | CUAGCAUU | UGUAGCUCCCUCC | GCmAUUmGUUGmUmAm | and | 10907708 |
| | | | GUUGCUACAAUAA | GmCUCCCmU(L1)mCm | 3181 | |
| | | | GGCCGUCGAUGUG | CGUUmGmCUAmCAAU* | | |
| | | | CCGCAACGCUCUG | AAGmGmCCmGmUmC(L | | |
| | | | CCGGCAUCGUU | 1)mGmAmUGUGCmCGm | | |
| | | | | CAAmCGCUCUmGmCC( | | |
| | | | | L1)GGCAUCGmU*mU | | |
| G023492 | 553 | GCCCUAUG | GCCCUAUGACAAC | mG*mC*mC*mCmUAUm | 2553, | chr16: |
| | | ACAACUGG | UGGCUGGAGGGGU | GmACmAAmCUGGCmUG | 3087 | 10907726- |
| | | CUGGAGGG | UGUAGCUCCCUCC | GAmGGGmGUUGmUmAm | and | 10907750 |
| | | | GUUGCUACAAUAA | GmCUCCCmU(L1)mCm | 3182 | |
| | | | GGCCGUCGAUGUG | CGUUmGmCUAmCAAU* | | |
| | | | CCGCAACGCUCUG | AAGmGmCCmGmUmC(L | | |
| | | | CCGGCAUCGUU | 1)mGmAmUGUGCmCGm | | |
| | | | | CAAmCGCUCUmGmCC( | | |
| | | | | L1)GGCAUCGmU*mU | | |
| G023493 | 554 | UGCCACGC | UGCCACGCUUUCU | mU*mG*mC*mCmACGm | 2554, | chr16: |
| | | UUUCUGGC | GGCUGGGCUGAGU | CmUUmUCmUGGCUmGG | 3088 | 10907752- |
| | | UGGGCUGA | UGUAGCUCCCUCC | GCmUGAmGUUGmUmAm | and | 10907776 |
| | | | GUUGCUACAAUAA | GmCUCCCmU(L1)mCm | 3183 | |
| | | | GGCCGUCGAUGUG | CGUUmGmCUAmCAAU* | | |
| | | | CCGCAACGCUCUG | AAGmGmCCmGmUmC(L | | |
| | | | CCGGCAUCGUU | 1)mGmAmUGUGCmCGm | | |
| | | | | CAAmCGCUCUmGmCC( | | |
| | | | | L1)GGCAUCGmU*mU | | |
| G023494 | 555 | ACGCUUUC | ACGCUUUCUGGCU | mA*mC*mG*mCmUUUm | 2555, | chr16: |
| | | UGGCUGGG | GGGCUGAUCUUGU | CmUGmGCmUGGGCmUG | 3089 | 10907756- |
| | | CUGAUCUU | UGUAGCUCCCUCC | AUmCUUmGUUGmUmAm | and | 10907780 |
| | | | GUUGCUACAAUAA | GmCUCCCmU(L1)mCm | 3184 | |
| | | | GGCCGUCGAUGUG | CGUUmGmCUAmCAAU* | | |
| | | | CCGCAACGCUCUG | AAGmGmCCmGmUmC(L | | |
| | | | CCGGCAUCGUU | 1)mGmAmUGUGCmCGm | | |
| | | | | CAAmCGCUCUmGmCC( | | |
| | | | | L1)GGCAUCGmU*mU | | |
| G023495 | 556 | CUUUCUGG | CUUUCUGGCUGGG | mC*mU*mU*mUmCUGm | 2556 | chr16: |
| | | CUGGGCUG | CUGAUCUUCCAGU | GmCUmGGmGCUGAmUC | 3090 | 10907759- |
| | | AUCUUCCA | UGUAGCUCCCUCC | UUmCCAmGUUGmUmAm | and | 10907783 |
| | | | GUUGCUACAAUAA | GmCUCCCmU(L1)mCm | 3185 | |
| | | | GGCCGUCGAUGUG | CGUUmGmCUAmCAAU* | | |
| | | | CCGCAACGCUCUG | AAGmGmCCmGmUmC(L | | |
| | | | CCGGCAUCGUU | 1)mGmAmUGUGCmCGm | | |
| | | | | CAAmCGCUCUmGmCC( | | |
| | | | | L1)GGCAUCGmU*mU | | |
| G023496 | 557 | CUGGCUGG | CUGGCUGGGCUGA | mC*mU*mG*mGmCUGm | 2557, | chr16: |
| | | GCUGAUCU | UCUUCCAGCCUGU | GmGCmUGmAUCUUmCC | 3091 | 10907763- |
| | | UCCAGCCU | UGUAGCUCCCUCC | AGmCCUmGUUGmUmAm | and | 10907787 |
| | | | GUUGCUACAAUAA | GmCUCCCmU(L1)mCm | 3186 | |
| | | | GGCCGUCGAUGUG | CGUUmGmCUAmCAAU* | | |
| | | | CCGCAACGCUCUG | AAGmGmCCmGmUmC(L | | |
| | | | CCGGCAUCGUU | 1)mGmAmUGUGCmCGm | | |
| | | | | CAAmCGCUCUmGmCC( | | |
| | | | | L1)GGCAUCGmU*mU | | |
| G023497 | 558 | UGGCUGGG | UGGCUGGGCUGAU | mU*mG*mG*mCmUGGm | 2558, | chr16: |
| | | CUGAUCUU | CUUCCAGCCUCGU | GmCUmGAmUCUUCmCA | 3092 | 10907764- |
| | | CCAGCCUC | UGUAGCUCCCUCC | GCmCUCmGUUGmUmAm | and | 10907788 |
| | | | GUUGCUACAAUAA | GmCUCCCmU(L1)mCm | 3187 | |
| | | | GGCCGUCGAUGUG | CGUUmGmCUAmCAAU* | | |
| | | | CCGCAACGCUCUG | AAGmGmCCmGmUmC(L | | |
| | | | CCGGCAUCGUU | 1)mGmAmUGUGCmCGm | | |
| | | | | CAAmCGCUCUmGmCC( | | |
| | | | | L1)GGCAUCGmU*mU | | |
| G023498 | 559 | GGAGCCCU | GGAGCCCUACUCG | mG*mG*mA*mGmCCCm | 2559, | chr16: |
| | | ACUCGGGC | GGCCAUCGGCGGU | UmACmUCmGGGCCmAU | 3093 | 10907802- |
| | | CAUCGGCG | UGUAGCUCCCUCC | CGmGCGmGUUGmUmAm | and | 10907826 |
| | | | GUUGCUACAAUAA | GmCUCCCmU(L1)mCm | 3188 | |
| | | | GGCCGUCGAUGUG | CGUUmGmCUAmCAAU* | | |
| | | | CCGCAACGCUCUG | AAGmGmCCmGmUmC(L | | |
| | | | CCGGCAUCGUU | 1)mGmAmUGUGCmCGm | | |
| | | | | CAAmCGCUCUmGmCC( | | |
| | | | | L1)GGCAUCGmU*mU | | |
| G023499 | 560 | ACAGGAAG | ACAGGAAGCAGAA | mA*mC*mA*mGmGAAm | 2560, | chr16: |
| | | CAGAAGGU | GGUGCUUGCGAGU | GmCAmGAmAGGUGmCU | 3094 | 10907839- |
| | | GCUUGCGA | UGUAGCUCCCUCC | UGmCGAmGUUGmUmAm | and | 10907863 |
| | | | GUUGCUACAAUAA | GmCUCCCmU(L1)mCm | 3189 | |
| | | | GGCCGUCGAUGUG | CGUUmGmCUAmCAAU* | | |
| | | | CCGCAACGCUCUG | AAGmGmCCmGmUmC(L | | |
| | | | CCGGCAUCGUU | 1)mGmAmUGUGCmCGm | | |
| | | | | CAAmCGCUCUmGmCC( | | |
| | | | | L1)GGCAUCGmU*mU | | |
| G023500 | 561 | GCUUGCGA | GCUUGCGAGGUAC | mG*mC*mU*mUmGCGm | 2561, | chr16: |
| | | GGUACCUG | CUGAAGCGGCUGU | AmGGmUAmCCUGAmAG | 3095 | 10907855- |
| | | AAGCGGCU | UGUAGCUCCCUCC | CGmGCUmGUUGmUmAm | and | 10907879 |
| | | | GUUGCUACAAUAA | GmCUCCCmU(L1)mCm | 3190 | |
| | | | GGCCGUCGAUGUG | CGUUmGmCUAmCAAU* | | |
| | | | CCGCAACGCUCUG | AAGmGmCCmGmUmC(L | | |
| | | | CCGGCAUCGUU | 1)mGmAmUGUGCmCGm | | |
| | | | | CAAmCGCUCUmGmCC( | | |
| | | | | L1)GGCAUCGmU*mU | | |
| G023501 | 562 | GAAUUUGG | GAAUUUGGCAGCA | mG*mA*mA*mUmUUGm | 2562, | chr16: |
| | | CAGCACGU | CGUGGUACAGGGU | GmCAmGCmACGUGmGU | 3096 | 10907950- |
| | | GGUACAGG | UGUAGCUCCCUCC | ACmAGGmGUUGmUmAm | and | 10907974 |
| | | | GUUGCUACAAUAA | GmCUCCCmU(L1)mCm | 3191 | |
| | | | GGCCGUCGAUGUG | CGUUmGmCUAmCAAU* | | |
| | | | CCGCAACGCUCUG | AAGmGmCCmGmUmC(L | | |
| | | | CCGGCAUCGUU | 1)mGmAmUGUGCmCGm | | |
| | | | | CAAmCGCUCUmGmCC( | | |
| | | | | L1)GGCAUCGmU*mU | | |
| G023502 | 563 | AAUUUGGC | AAUUUGGCAGCAC | mA*mA*mU*mUmUGGm | 2563, | chr16: |
| | | AGCACGUG | GUGGUACAGGAGU | CmAGmCAmCGUGGmUA | 3097 | 10907951- |
| | | GUACAGGA | UGUAGCUCCCUCC | CAmGGAmGUUGmUmAm | and | 10907975 |
| | | | GUUGCUACAAUAA | GmCUCCCmU(L1)mCm | 3192 | |
| | | | GGCCGUCGAUGUG | CGUUmGmCUAmCAAU* | | |
| | | | CCGCAACGCUCUG | AAGmGmCCmGmUmC(L | | |
| | | | CCGGCAUCGUU | 1)mGmAmUGUGCmCGm | | |
| | | | | CAAmCGCUCUmGmCC( | | |
| | | | | L1)GGCAUCGmU*mU | | |
| G023503 | 564 | AUUUGGCA | AUUUGGCAGCACG | mA*mU*mU*mUmGGCm | 2564, | chr16: |
| | | GCACGUGG | UGGUACAGGAGGU | AmGCmACmGUGGUmAC | 3098 | 10907952- |
| | | UACAGGAG | UGUAGCUCCCUCC | AGmGAGmGUUGmUmAm | and | 10907976 |
| | | | GUUGCUACAAUAA | GmCUCCCmU(L1)mCm | 3193 | |
| | | | GGCCGUCGAUGUG | CGUUmGmCUAmCAAU* | | |
| | | | CCGCAACGCUCUG | AAGmGmCCmGmUmC(L | | |
| | | | CCGGCAUCGUU | 1)mGmAmUGUGCmCGm | | |
| | | | | CAAmCGCUCUmGmCC( | | |
| | | | | L1)GGCAUCGmU*mU | | |
| G023504 | 565 | CCGGCCGC | CCGGCCGCCUCUC | mC*mC*mG*mGmCCGm | 2565, | chr16: |
| | | CUCUCUUU | UUUUCUGGGCAGU | CmCUmCUmCUUUUmCU | 3099 | 10907980- |
| | | UCUGGGCA | UGUAGCUCCCUCC | GGmGCAmGUUGmUmAm | and | 10908004 |
| | | | GUUGCUACAAUAA | GmCUCCCmU(L1)mCm | 3194 | |
| | | | GGCCGUCGAUGUG | CGUUmGmCUAmCAAU* | | |
| | | | CCGCAACGCUCUG | AAGmGmCCmGmUmC(L | | |
| | | | CCGGCAUCGUU | 1)mGmAmUGUGCmCGm | | |
| | | | | CAAmCGCUCUmGmCC( | | |
| | | | | L1)GGCAUCGmU*mU | | |
| G023505 | 566 | CCUCUCUU | CCUCUCUUUUCUG | mC*mC*mU*mCmUCUm | 2566, | chr16: |
| | | UUCUGGGC | GGCACCCGCCUGU | UmUUmCUmGGGCAmCC | 3100 | 10907987- |
| | | ACCCGCCU | UGUAGCUCCCUCC | CGmCCUmGUUGmUmAm | and | 10908011 |
| | | | GUUGCUACAAUAA | GmCUCCCmU(L1)mCm | 3195 | |
| | | | GGCCGUCGAUGUG | CGUUmGmCUAmCAAU* | | |
| | | | CCGCAACGCUCUG | AAGmGmCCmGmUmC(L | | |
| | | | CCGGCAUCGUU | 1)mGmAmUGUGCmCGm | | |
| | | | | CAAmCGCUCUmGmCC( | | |
| | | | | L1)GGCAUCGmU*mU | | |
| G023506 | 567 | CCUCCUGA | CCUCCUGAUGCAC | mC*mC*mU*mCmCUGm | 2567, | chr16: |
| | | UGCACAUG | AUGUACUGGGCGU | AmUGmCAmCAUGUmAC | 3101 | 10908015- |
| | | UACUGGGC | UGUAGCUCCCUCC | UGmGGCmGUUGmUmAm | and | 10908039 |
| | | | GUUGCUACAAUAA | GmCUCCCmU(L1)mCm | 3196 | |
| | | | GGCCGUCGAUGUG | CGUUmGmCUAmCAAU* | | |
| | | | CCGCAACGCUCUG | AAGmGmCCmGmUmC(L | | |
| | | | CCGGCAUCGUU | 1)mGmAmUGUGCmCGm | | |
| | | | | CAAmCGCUCUmGmCC( | | |
| | | | | L1)GGCAUCGmU*mU | | |
| G023507 | 568 | GCCUUGGA | GCCUUGGAGGCGG | mG*mC*mC*mUmUGGm | 2568, | chr16: |
| | | GGCGGCGG | CGGGCCAAGACGU | AmGGmCGmGCGGGmCC | 3102 | 10908042- |
| | | GCCAAGAC | UGUAGCUCCCUCC | AAmGACmGUUGmUmAm | and | 10908066 |
| | | | GUUGCUACAAUAA | GmCUCCCmU(L1)mCm | 3197 | |
| | | | GGCCGUCGAUGUG | CGUUmGmCUAmCAAU* | | |
| | | | CCGCAACGCUCUG | AAGmGmCCmGmUmC(L | | |
| | | | CCGGCAUCGUU | 1)mGmAmUGUGCmCGm | | |
| | | | | CAAmCGCUCUmGmCC( | | |
| | | | | L1)GGCAUCGmU*mU | | |
| G023508 | 569 | CCUUGGAG | CCUUGGAGGCGGC | mC*mC*mU*mUmGGAm | 2569, | chr16: |
| | | GCGGCGGG | GGGCCAAGACUGU | GmGCmGGmCGGGCmCA | 3103 | 10908043- |
| | | CCAAGACU | UGUAGCUCCCUCC | AGmACUmGUUGmUmAm | and | 10908067 |
| | | | GUUGCUACAAUAA | GmCUCCCmU(L1)mCm | 3198 | |
| | | | GGCCGUCGAUGUG | CGUUmGmCUAmCAAU* | | |
| | | | CCGCAACGCUCUG | AAGmGmCCmGmUmC(L | | |
| | | | CCGGCAUCGUU | 1)mGmAmUGUGCmCGm | | |
| | | | | CAAmCGCUCUmGmCC( | | |
| | | | | L1)GGCAUCGmU*mU | | |
| G023509 | 570 | AGGCGGCG | AGGCGGCGGGCCA | mA*mG*mG*mCmGGCm | 2570, | chr16: |
| | | GGCCAAGA | AGACUUCUCCCGU | GmGGmCCmAAGACmUU | 3104 | 10908049- |
| | | CUUCUCCC | UGUAGCUCCCUCC | CUmCCCmGUUGmUmAm | and | 10908073 |
| | | | GUUGCUACAAUAA | GmCUCCCmU(L1)mCm | 3199 | |
| | | | GGCCGUCGAUGUG | CGUUmGmCUAmCAAU* | | |
| | | | CCGCAACGCUCUG | AAGmGmCCmGmUmC(L | | |
| | | | CCGGCAUCGUU | 1)mGmAmUGUGCmCGm | | |
| | | | | CAAmCGCUCUmGmCC( | | |
| | | | | L1)GGCAUCGmU*mU | | |
| G023510 | 571 | CGGCGGGC | CGGCGGGCCAAGA | mC*mG*mG*mCmGGGm | 2571, | chr16: |
| | | CAAGACUU | CUUCUCCCUGGGU | CmCAmAGmACUUCmUC | 3105 | 10908052- |
| | | CUCCCUGG | UGUAGCUCCCUCC | CCmUGGmGUUGmUmAm | and | 10908076 |
| | | | GUUGCUACAAUAA | GmCUCCCmU(L1)mCm | 3200 | |
| | | | GGCCGUCGAUGUG | CGUUmGmCUAmCAAU* | | |
| | | | CCGCAACGCUCUG | AAGmGmCCmGmUmC(L | | |
| | | | CCGGCAUCGUU | 1)mGmAmUGUGCmCGm | | |
| | | | | CAAmCGCUCUmGmCC( | | |
| | | | | L1)GGCAUCGmU*mU | | |
| G023511 | 572 | CCCUGGAC | CCCUGGACCUCCG | mC*mC*mC*mUmGGAm | 2572, | chr16: |
| | | CUCCGCAG | CAGCACUGGCAGU | CmCUmCCmGCAGCmAC | 3106 | 10908070- |
| | | CACUGGCA | UGUAGCUCCCUCC | UGmGCAmGUUGmUmAm | and | 10908094 |
| | | | GUUGCUACAAUAA | GmCUCCCmU(L1)mCm | 3201 | |
| | | | GGCCGUCGAUGUG | CGUUmGmCUAmCAAU* | | |
| | | | CCGCAACGCUCUG | AAGmGmCCmGmUmC(L | | |
| | | | CCGGCAUCGUU | 1)mGmAmUGUGCmCGm | | |
| | | | | CAAmCGCUCUmGmCC( | | |
| | | | | L1)GGCAUCGmU*mU | | |
| G023512 | 573 | CCUGGACC | CCUGGACCUCCGC | mC*mC*mU*mGmGACm | 2573, | chr16: |
| | | UCCGCAGC | AGCACUGGCAUGU | CmUCmCGmCAGCAmCU | 3107 | 10908071- |
| | | ACUGGCAU | UGUAGCUCCCUCC | GGmCAUmGUUGmUmAm | and | 10908095 |
| | | | GUUGCUACAAUAA | GmCUCCCmU(L1)mCm | 3202 | |
| | | | GGCCGUCGAUGUG | CGUUmGmCUAmCAAU* | | |
| | | | CCGCAACGCUCUG | AAGmGmCCmGmUmC(L | | |
| | | | CCGGCAUCGUU | 1)mGmAmUGUGCmCGm | | |
| | | | | CAAmCGCUCUmGmCC( | | |
| | | | | L1)GGCAUCGmU*mU | | |
| G023513 | 574 | CUGGACCU | CUGGACCUCCGCA | mC*mU*mG*mGmACCm | 2574, | chr16: |
| | | CCGCAGCA | GCACUGGCAUUGU | UmCCmGCmAGCACmUG | 3108 | 10908072- |
| | | CUGGCAUU | UGUAGCUCCCUCC | GCmAUUmGUUGmUmAm | and | 10908096 |
| | | | GUUGCUACAAUAA | GmCUCCCmU(L1)mCm | 3203 | |
| | | | GGCCGUCGAUGUG | CGUUmGmCUAmCAAU* | | |
| | | | CCGCAACGCUCUG | AAGmGmCCmGmUmC(L | | |
| | | | CCGGCAUCGUU | 1)mGmAmUGUGCmCGm | | |
| | | | | CAAmCGCUCUmGmCC( | | |
| | | | | L1)GGCAUCGmU*mU | | |
| G023514 | 575 | GGGAGCCU | GGGAGCCUCGUGG | mG*mG*mG*mAmGCCm | 2575, | chr16: |
| | | CGUGGGAC | GACUCAGCUGUGU | UmCGmUGmGGACUmCA | 3109 | 10908111- |
| | | UCAGCUGU | UGUAGCUCCCUCC | GCmUGUmGUUGmUmAm | and | 10908135 |
| | | | GUUGCUACAAUAA | GmCUCCCmU(L1)mCm | 3204 | |
| | | | GGCCGUCGAUGUG | CGUUmGmCUAmCAAU* | | |
| | | | CCGCAACGCUCUG | AAGmGmCCmGmUmC(L | | |
| | | | CCGGCAUCGUU | 1)mGmAmUGUGCmCGm | | |
| | | | | CAAmCGCUCUmGmCC( | | |
| | | | | L1)GGCAUCGmU*mU | | |
| G023515 | 576 | GGAGCCUC | GGAGCCUCGUGGG | mG*mG*mA*mGmCCUm | 2576, | chr16: |
| | | GUGGGACU | ACUCAGCUGUGGU | CmGUmGGmGACUCmAG | 3110 | 10908112- |
| | | CAGCUGUG | UGUAGCUCCCUCC | CUmGUGmGUUGmUmAm | and | 10908136 |
| | | | GUUGCUACAAUAA | GmCUCCCmU(L1)mCm | 3205 | |
| | | | GGCCGUCGAUGUG | CGUUmGmCUAmCAAU* | | |
| | | | CCGCAACGCUCUG | AAGmGmCCmGmUmC(L | | |
| | | | CCGGCAUCGUU | 1)mGmAmUGUGCmCGm | | |
| | | | | CAAmCGCUCUmGmCC( | | |
| | | | | L1)GGCAUCGmU*mU | | |

The terms “mA,” “mC,” “mU,” or “mG” may be used to denote a nucleotide that has been modified with 2′—O-Me. The terms A*, C*, U*, or G* may be used to denote a nucleotide that is linked to the next (e.g., 3′) nucleotide with a phosphorothioate (PS) bond. As used herein, the “(L1)” refers to an internal linker having a bridging length of about 15-21 atoms (e.g., about 18 atoms) as described below, e.g., see Table 28.

In some embodiments, the CIITA guide RNA comprises a guide sequence selected from SEQ TD NOs: 301, 302, 304-576. In some embodiments, the CIITA guide RNA comprises a guide sequence that is at least 19, 20, 21, 22, 23, or 24 contiguous nucleotides of a sequence selected from SEQ ID NOs: 301, 302, 304-576. In some embodiments, the CIITA guide RNA comprises a guide sequence that is at least 95%, 90%, or 85% identical to a sequence selected from SEQ ID NOs: 301, 302, 304-576. In some embodiments, the CIITA guide RNA comprises a guide sequence that is at least 95% identical to a sequence selected from SEQ ID NOs: 301, 302, 304-576.

In some embodiments, the CIITA guide RNA comprises a guide sequence that comprises at least 10 contiguous nucleotides±10 nucleotides of a genomic coordinate listed in Table 4. As used herein, at least 10 contiguous nucleotides±10 nucleotides of a genomic coordinate means, for example, at least 10 contiguous nucleotides within the genomic coordinates wherein the genomic coordinates include 10 nucleotides in the 5′ direction and 10 nucleotides in the 3′ direction from the ranges listed in Table 4. For example, a CIITA guide RNA may comprise 10 contiguous nucleotides within the genomic coordinates chosen from: (a) chr16:10907504-10907528; chr16:10907508-10907532; chr16:10907539-10907559; chr16:10895658-10895682; chr16:10895668-10895692; chr16:10895750-10895774; chr16:10895753-10895777; chr16:10895754-10895778; chr16:10898684-10898708; chr16:10901529-10901553; chr16:10902121-10902145; chr16:10902701-10902725; chr16:10904726-10904750; chr16:10904760-10904784; chr16:10906493-10906517; chr16:10906515-10906539; chr16:10906631-10906655; chr16:10906636-10906660; chr16:10906643-10906667; chr16:10906770-10906794; chr16:10906788-10906812; chr16:10906789-10906813; chr16:10906816-10906840; chr16:10907148-10907172; chr16:10907254-10907278; chr16:10907331-10907355; chr16:10907477-10907501; chr16:10907497-10907521; chr16:10907503-10907527; and chr16:10907574-10907598; or (b) chr16:10906889-10906913; and chr16:10907504-10907528; including the boundary nucleotides of these ranges. In some embodiments, the CIITA guide RNA comprises a guide sequence that is at least 19, 20, 21, 22, 23, or 24 contiguous nucleotides of a sequence that comprises 10 contiguous nucleotides±10 nucleotides of a genomic coordinate listed in Table 4. In some embodiments, the CIITA guide RNA comprises a guide sequence that is at least 95%, 90%, or 85% identical to a sequence selected from a sequence that is 19, 20, 21, 22, 23, or 24 contiguous nucleotides of a sequence that comprises 10 contiguous nucleotides±10 nucleotides of a genomic coordinate listed in Table 4.

In some embodiments, the CIITA guide RNA comprises a guide sequence that comprises at least 15 contiguous nucleotides±10 nucleotides of a genomic coordinate listed in Table 4. In some embodiments, the CIITA guide RNA comprises a guide sequence that comprises at least 24 contiguous nucleotides±10 nucleotides of a genomic coordinate listed in Table 4.

In some embodiments, the CIITA guide RNA comprises SEQ ID NO: 301. In some embodiments, the CIITA guide RNA comprises SEQ ID NO: 302. In some embodiments, the CIITA guide RNA comprises SEQ ID NO: 304. In some embodiments, the CIITA guide RNA comprises SEQ ID NO: 305. In some embodiments, the CIITA guide RNA comprises SEQ ID NO: 306. In some embodiments, the CIITA guide RNA comprises SEQ ID NO: 307. In some embodiments, the CIITA guide RNA comprises SEQ ID NO: 308. In some embodiments, the CIITA guide RNA comprises SEQ ID NO: 309. In some embodiments, the CIITA guide RNA comprises SEQ ID NO: 310. In some embodiments, the CIITA guide RNA comprises SEQ ID NO: 311. In some embodiments, the CIITA guide RNA comprises SEQ ID NO: 312. In some embodiments, the CIITA guide RNA comprises SEQ ID NO: 313. In some embodiments, the CIITA guide RNA comprises SEQ ID NO: 314. In some embodiments, the CIITA guide RNA comprises SEQ ID NO: 315. In some embodiments, the CIITA guide RNA comprises SEQ ID NO: 316. In some embodiments, the CIITA guide RNA comprises SEQ ID NO: 317. In some embodiments, the CIITA guide RNA comprises SEQ ID NO: 318. In some embodiments, the CIITA guide RNA comprises SEQ ID NO: 319. In some embodiments, the CIITA guide RNA comprises SEQ ID NO: 320. In some embodiments, the CIITA guide RNA comprises SEQ ID NO: 321. In some embodiments, the CIITA guide RNA comprises SEQ ID NO: 322. In some embodiments, the CIITA guide RNA comprises SEQ ID NO: 323. In some embodiments, the CIITA guide RNA comprises SEQ ID NO: 324. In some embodiments, the CIITA guide RNA comprises SEQ ID NO: 325. In some embodiments, the CIITA guide RNA comprises SEQ ID NO: 326. In some embodiments, the CIITA guide RNA comprises SEQ ID NO: 327. In some embodiments, the CIITA guide RNA comprises SEQ ID NO: 328. In some embodiments, the CIITA guide RNA comprises SEQ ID NO: 329. In some embodiments, the CIITA guide RNA comprises SEQ ID NO: 330. In some embodiments, the CIITA guide RNA comprises SEQ ID NO: 331. In some embodiments, the CIITA guide RNA comprises SEQ ID NO: 332. In some embodiments, the CIITA guide RNA comprises SEQ ID NO: 333. In some embodiments, the CIITA guide RNA comprises SEQ ID NO: 334. In some embodiments, the CIITA guide RNA comprises SEQ ID NO: 335. In some embodiments, the CIITA guide RNA comprises SEQ ID NO: 336. In some embodiments, the CIITA guide RNA comprises SEQ ID NO: 337. In some embodiments, the CIITA guide RNA comprises SEQ ID NO: 338. In some embodiments, the CIITA guide RNA comprises SEQ ID NO: 339. In some embodiments, the CIITA guide RNA comprises SEQ ID NO: 340. In some embodiments, the CIITA guide RNA comprises SEQ ID NO: 341. In some embodiments, the CIITA guide RNA comprises SEQ ID NO: 342. In some embodiments, the CIITA guide RNA comprises SEQ ID NO: 343. In some embodiments, the CIITA guide RNA comprises SEQ ID NO: 344. In some embodiments, the CIITA guide RNA comprises SEQ ID NO: 345. In some embodiments, the CIITA guide RNA comprises SEQ ID NO: 346. In some embodiments, the CIITA guide RNA comprises SEQ ID NO: 347. In some embodiments, the CIITA guide RNA comprises SEQ ID NO: 348. In some embodiments, the CIITA guide RNA comprises SEQ ID NO: 349. In some embodiments, the CIITA guide RNA comprises SEQ ID NO: 350. In some embodiments, the CIITA guide RNA comprises SEQ ID NO: 351. In some embodiments, the CIITA guide RNA comprises SEQ ID NO: 352. In some embodiments, the CIITA guide RNA comprises SEQ ID NO: 353. In some embodiments, the CIITA guide RNA comprises SEQ ID NO: 354. In some embodiments, the CIITA guide RNA comprises SEQ ID NO: 355. In some embodiments, the CIITA guide RNA comprises SEQ ID NO: 356. In some embodiments, the CIITA guide RNA comprises SEQ ID NO: 357. In some embodiments, the CIITA guide RNA comprises SEQ ID NO: 358. In some embodiments, the CIITA guide RNA comprises SEQ ID NO: 359. In some embodiments, the CIITA guide RNA comprises SEQ ID NO: 360. In some embodiments, the CIITA guide RNA comprises SEQ ID NO: 361. In some embodiments, the CIITA guide RNA comprises SEQ ID NO: 362. In some embodiments, the CIITA guide RNA comprises SEQ ID NO: 363. In some embodiments, the CIITA guide RNA comprises SEQ ID NO: 364. In some embodiments, the CIITA guide RNA comprises SEQ ID NO: 365. In some embodiments, the CIITA guide RNA comprises SEQ ID NO: 366. In some embodiments, the CIITA guide RNA comprises SEQ ID NO: 367. In some embodiments, the CIITA guide RNA comprises SEQ ID NO: 368. In some embodiments, the CIITA guide RNA comprises SEQ ID NO: 369. In some embodiments, the CIITA guide RNA comprises SEQ ID NO: 370. In some embodiments, the CIITA guide RNA comprises SEQ ID NO: 371. In some embodiments, the CIITA guide RNA comprises SEQ ID NO: 372. In some embodiments, the CIITA guide RNA comprises SEQ ID NO: 373. In some embodiments, the CIITA guide RNA comprises SEQ ID NO: 374. In some embodiments, the CIITA guide RNA comprises SEQ ID NO: 375. In some embodiments, the CIITA guide RNA comprises SEQ ID NO: 376. In some embodiments, the CIITA guide RNA comprises SEQ ID NO: 377. In some embodiments, the CIITA guide RNA comprises SEQ ID NO: 378. In some embodiments, the CIITA guide RNA comprises SEQ ID NO: 379. In some embodiments, the CIITA guide RNA comprises SEQ ID NO: 380. In some embodiments, the CIITA guide RNA comprises SEQ ID NO: 381. In some embodiments, the CIITA guide RNA comprises SEQ ID NO: 382. In some embodiments, the CIITA guide RNA comprises SEQ ID NO: 383. In some embodiments, the CIITA guide RNA comprises SEQ ID NO: 384. In some embodiments, the CIITA guide RNA comprises SEQ ID NO: 385. In some embodiments, the CIITA guide RNA comprises SEQ ID NO: 386. In some embodiments, the CIITA guide RNA comprises SEQ ID NO: 387. In some embodiments, the CIITA guide RNA comprises SEQ ID NO: 388. In some embodiments, the CIITA guide RNA comprises SEQ ID NO: 389. In some embodiments, the CIITA guide RNA comprises SEQ ID NO: 390. In some embodiments, the CIITA guide RNA comprises SEQ ID NO: 391. In some embodiments, the CIITA guide RNA comprises SEQ ID NO: 392. In some embodiments, the CIITA guide RNA comprises SEQ ID NO: 393. In some embodiments, the CIITA guide RNA comprises SEQ ID NO: 394. In some embodiments, the CIITA guide RNA comprises SEQ ID NO: 395. In some embodiments, the CIITA guide RNA comprises SEQ ID NO: 396. In some embodiments, the CIITA guide RNA comprises SEQ ID NO: 397. In some embodiments, the CIITA guide RNA comprises SEQ ID NO: 398. In some embodiments, the CIITA guide RNA comprises SEQ ID NO: 399. In some embodiments, the CIITA guide RNA comprises SEQ ID NO: 400. In some embodiments, the CIITA guide RNA comprises SEQ ID NO: 401. In some embodiments, the CIITA guide RNA comprises SEQ ID NO: 402. In some embodiments, the CIITA guide RNA comprises SEQ ID NO: 403. In some embodiments, the CIITA guide RNA comprises SEQ ID NO: 404. In some embodiments, the CIITA guide RNA comprises SEQ ID NO: 405. In some embodiments, the CIITA guide RNA comprises SEQ ID NO: 406. In some embodiments, the CIITA guide RNA comprises SEQ ID NO: 407. In some embodiments, the CIITA guide RNA comprises SEQ ID NO: 408. In some embodiments, the CIITA guide RNA comprises SEQ ID NO: 409. In some embodiments, the CIITA guide RNA comprises SEQ ID NO: 410. In some embodiments, the CIITA guide RNA comprises SEQ ID NO: 411. In some embodiments, the CIITA guide RNA comprises SEQ ID NO: 412. In some embodiments, the CIITA guide RNA comprises SEQ ID NO: 413. In some embodiments, the CIITA guide RNA comprises SEQ ID NO: 414. In some embodiments, the CIITA guide RNA comprises SEQ ID NO: 415. In some embodiments, the CIITA guide RNA comprises SEQ ID NO: 416. In some embodiments, the CIITA guide RNA comprises SEQ ID NO: 417. In some embodiments, the CIITA guide RNA comprises SEQ ID NO: 418. In some embodiments, the CIITA guide RNA comprises SEQ ID NO: 419. In some embodiments, the CIITA guide RNA comprises SEQ ID NO: 420. In some embodiments, the CIITA guide RNA comprises SEQ ID NO: 421. In some embodiments, the CIITA guide RNA comprises SEQ ID NO: 422. In some embodiments, the CIITA guide RNA comprises SEQ ID NO: 423. In some embodiments, the CIITA guide RNA comprises SEQ ID NO: 424. In some embodiments, the CIITA guide RNA comprises SEQ ID NO: 425. In some embodiments, the CIITA guide RNA comprises SEQ ID NO: 426. In some embodiments, the CIITA guide RNA comprises SEQ ID NO: 427. In some embodiments, the CIITA guide RNA comprises SEQ ID NO: 428. In some embodiments, the CIITA guide RNA comprises SEQ ID NO: 429. In some embodiments, the CIITA guide RNA comprises SEQ ID NO: 430. In some embodiments, the CIITA guide RNA comprises SEQ ID NO: 431. In some embodiments, the CIITA guide RNA comprises SEQ ID NO: 432. In some embodiments, the CIITA guide RNA comprises SEQ ID NO: 433. In some embodiments, the CIITA guide RNA comprises SEQ ID NO: 434. In some embodiments, the CIITA guide RNA comprises SEQ ID NO: 435. In some embodiments, the CIITA guide RNA comprises SEQ ID NO: 436. In some embodiments, the CIITA guide RNA comprises SEQ ID NO: 437. In some embodiments, the CIITA guide RNA comprises SEQ ID NO: 438. In some embodiments, the CIITA guide RNA comprises SEQ ID NO: 439. In some embodiments, the CIITA guide RNA comprises SEQ ID NO: 440. In some embodiments, the CIITA guide RNA comprises SEQ ID NO: 441. In some embodiments, the CIITA guide RNA comprises SEQ ID NO: 442. In some embodiments, the CIITA guide RNA comprises SEQ ID NO: 443. In some embodiments, the CIITA guide RNA comprises SEQ ID NO: 444. In some embodiments, the CIITA guide RNA comprises SEQ ID NO: 445. In some embodiments, the CIITA guide RNA comprises SEQ ID NO: 446. In some embodiments, the CIITA guide RNA comprises SEQ ID NO: 447. In some embodiments, the CIITA guide RNA comprises SEQ ID NO: 448. In some embodiments, the CIITA guide RNA comprises SEQ ID NO: 449. In some embodiments, the CIITA guide RNA comprises SEQ ID NO: 450. In some embodiments, the CIITA guide RNA comprises SEQ ID NO: 451. In some embodiments, the CIITA guide RNA comprises SEQ ID NO: 452. In some embodiments, the CIITA guide RNA comprises SEQ ID NO: 453. In some embodiments, the CIITA guide RNA comprises SEQ ID NO: 454. In some embodiments, the CIITA guide RNA comprises SEQ ID NO: 455. In some embodiments, the CIITA guide RNA comprises SEQ ID NO: 456. In some embodiments, the CIITA guide RNA comprises SEQ ID NO: 457. In some embodiments, the CIITA guide RNA comprises SEQ ID NO: 458. In some embodiments, the CIITA guide RNA comprises SEQ ID NO: 459. In some embodiments, the CIITA guide RNA comprises SEQ ID NO: 460. In some embodiments, the CIITA guide RNA comprises SEQ ID NO: 461. In some embodiments, the CIITA guide RNA comprises SEQ ID NO: 462. In some embodiments, the CIITA guide RNA comprises SEQ ID NO: 463. In some embodiments, the CIITA guide RNA comprises SEQ ID NO: 464. In some embodiments, the CIITA guide RNA comprises SEQ ID NO: 465. In some embodiments, the CIITA guide RNA comprises SEQ ID NO: 466. In some embodiments, the CIITA guide RNA comprises SEQ ID NO: 467. In some embodiments, the CIITA guide RNA comprises SEQ ID NO: 468. In some embodiments, the CIITA guide RNA comprises SEQ ID NO: 469. In some embodiments, the CIITA guide RNA comprises SEQ ID NO: 470. In some embodiments, the CIITA guide RNA comprises SEQ ID NO: 471. In some embodiments, the CIITA guide RNA comprises SEQ ID NO: 472. In some embodiments, the CIITA guide RNA comprises SEQ ID NO: 473. In some embodiments, the CIITA guide RNA comprises SEQ ID NO: 474. In some embodiments, the CIITA guide RNA comprises SEQ ID NO: 475. In some embodiments, the CIITA guide RNA comprises SEQ ID NO: 476. In some embodiments, the CIITA guide RNA comprises SEQ ID NO: 477. In some embodiments, the CIITA guide RNA comprises SEQ ID NO: 478. In some embodiments, the CIITA guide RNA comprises SEQ ID NO: 479. In some embodiments, the CIITA guide RNA comprises SEQ ID NO: 480. In some embodiments, the CIITA guide RNA comprises SEQ ID NO: 481. In some embodiments, the CIITA guide RNA comprises SEQ ID NO: 482. In some embodiments, the CIITA guide RNA comprises SEQ ID NO: 483. In some embodiments, the CIITA guide RNA comprises SEQ ID NO: 484. In some embodiments, the CIITA guide RNA comprises SEQ ID NO: 485. In some embodiments, the CIITA guide RNA comprises SEQ ID NO: 486. In some embodiments, the CIITA guide RNA comprises SEQ ID NO: 487. In some embodiments, the CIITA guide RNA comprises SEQ ID NO: 488. In some embodiments, the CIITA guide RNA comprises SEQ ID NO: 489. In some embodiments, the CIITA guide RNA comprises SEQ ID NO: 490. In some embodiments, the CIITA guide RNA comprises SEQ ID NO: 491. In some embodiments, the CIITA guide RNA comprises SEQ ID NO: 492. In some embodiments, the CIITA guide RNA comprises SEQ ID NO: 493. In some embodiments, the CIITA guide RNA comprises SEQ ID NO: 494. In some embodiments, the CIITA guide RNA comprises SEQ ID NO: 495. In some embodiments, the CIITA guide RNA comprises SEQ ID NO: 496. In some embodiments, the CIITA guide RNA comprises SEQ ID NO: 497. In some embodiments, the CIITA guide RNA comprises SEQ ID NO: 498. In some embodiments, the CIITA guide RNA comprises SEQ ID NO: 499. In some embodiments, the CIITA guide RNA comprises SEQ ID NO: 500. In some embodiments, the CIITA guide RNA comprises SEQ ID NO: 501. In some embodiments, the CIITA guide RNA comprises SEQ ID NO: 502. In some embodiments, the CIITA guide RNA comprises SEQ ID NO: 503. In some embodiments, the CIITA guide RNA comprises SEQ ID NO: 504. In some embodiments, the CIITA guide RNA comprises SEQ ID NO: 505. In some embodiments, the CIITA guide RNA comprises SEQ ID NO: 506. In some embodiments, the CIITA guide RNA comprises SEQ ID NO: 507. In some embodiments, the CIITA guide RNA comprises SEQ ID NO: 508. In some embodiments, the CIITA guide RNA comprises SEQ ID NO: 509. In some embodiments, the CIITA guide RNA comprises SEQ ID NO: 510. In some embodiments, the CIITA guide RNA comprises SEQ ID NO: 511. In some embodiments, the CIITA guide RNA comprises SEQ ID NO: 512. In some embodiments, the CIITA guide RNA comprises SEQ ID NO: 513. In some embodiments, the CIITA guide RNA comprises SEQ ID NO: 514. In some embodiments, the CIITA guide RNA comprises SEQ ID NO: 515. In some embodiments, the CIITA guide RNA comprises SEQ ID NO: 516. In some embodiments, the CIITA guide RNA comprises SEQ ID NO: 517. In some embodiments, the CIITA guide RNA comprises SEQ ID NO: 518. In some embodiments, the CIITA guide RNA comprises SEQ ID NO: 519. In some embodiments, the CIITA guide RNA comprises SEQ ID NO: 520. In some embodiments, the CIITA guide RNA comprises SEQ ID NO: 521. In some embodiments, the CIITA guide RNA comprises SEQ ID NO: 522. In some embodiments, the CIITA guide RNA comprises SEQ ID NO: 523. In some embodiments, the CIITA guide RNA comprises SEQ ID NO: 524. In some embodiments, the CIITA guide RNA comprises SEQ ID NO: 525. In some embodiments, the CIITA guide RNA comprises SEQ ID NO: 526. In some embodiments, the CIITA guide RNA comprises SEQ ID NO: 527. In some embodiments, the CIITA guide RNA comprises SEQ ID NO: 528. In some embodiments, the CIITA guide RNA comprises SEQ ID NO: 529. In some embodiments, the CIITA guide RNA comprises SEQ ID NO: 530. In some embodiments, the CIITA guide RNA comprises SEQ ID NO: 531. In some embodiments, the CIITA guide RNA comprises SEQ ID NO: 532. In some embodiments, the CIITA guide RNA comprises SEQ ID NO: 533. In some embodiments, the CIITA guide RNA comprises SEQ ID NO: 534. In some embodiments, the CIITA guide RNA comprises SEQ ID NO: 535. In some embodiments, the CIITA guide RNA comprises SEQ ID NO: 536. In some embodiments, the CIITA guide RNA comprises SEQ ID NO: 537. In some embodiments, the CIITA guide RNA comprises SEQ ID NO: 538. In some embodiments, the CIITA guide RNA comprises SEQ ID NO: 539. In some embodiments, the CIITA guide RNA comprises SEQ ID NO: 540. In some embodiments, the CIITA guide RNA comprises SEQ ID NO: 541. In some embodiments, the CIITA guide RNA comprises SEQ ID NO: 542. In some embodiments, the CIITA guide RNA comprises SEQ ID NO: 543. In some embodiments, the CIITA guide RNA comprises SEQ ID NO: 544. In some embodiments, the CIITA guide RNA comprises SEQ ID NO: 545. In some embodiments, the CIITA guide RNA comprises SEQ ID NO: 546. In some embodiments, the CIITA guide RNA comprises SEQ ID NO: 547. In some embodiments, the CIITA guide RNA comprises SEQ ID NO: 548. In some embodiments, the CIITA guide RNA comprises SEQ ID NO: 549. In some embodiments, the CIITA guide RNA comprises SEQ ID NO: 550. In some embodiments, the CIITA guide RNA comprises SEQ ID NO: 551. In some embodiments, the CIITA guide RNA comprises SEQ ID NO: 552. In some embodiments, the CIITA guide RNA comprises SEQ ID NO: 553. In some embodiments, the CIITA guide RNA comprises SEQ ID NO: 554. In some embodiments, the CIITA guide RNA comprises SEQ ID NO: 555. In some embodiments, the CIITA guide RNA comprises SEQ ID NO: 556. In some embodiments, the CIITA guide RNA comprises SEQ ID NO: 557. In some embodiments, the CIITA guide RNA comprises SEQ ID NO: 558. In some embodiments, the CIITA guide RNA comprises SEQ ID NO: 559. In some embodiments, the CIITA guide RNA comprises SEQ ID NO: 560. In some embodiments, the CIITA guide RNA comprises SEQ ID NO: 561. In some embodiments, the CIITA guide RNA comprises SEQ ID NO: 562. In some embodiments, the CIITA guide RNA comprises SEQ ID NO: 563. In some embodiments, the CIITA guide RNA comprises SEQ ID NO: 564. In some embodiments, the CIITA guide RNA comprises SEQ ID NO: 565. In some embodiments, the CIITA guide RNA comprises SEQ ID NO: 566. In some embodiments, the CIITA guide RNA comprises SEQ ID NO: 567. In some embodiments, the CIITA guide RNA comprises SEQ ID NO: 568. In some embodiments, the CIITA guide RNA comprises SEQ ID NO: 569. In some embodiments, the CIITA guide RNA comprises SEQ ID NO: 570. In some embodiments, the CIITA guide RNA comprises SEQ ID NO: 571. In some embodiments, the CIITA guide RNA comprises SEQ ID NO: 572. In some embodiments, the CIITA guide RNA comprises SEQ ID NO: 573. In some embodiments, the CIITA guide RNA comprises SEQ ID NO: 574. In some embodiments, the CIITA guide RNA comprises SEQ ID NO: 575. In some embodiments, the CIITA guide RNA comprises SEQ ID NO: 576.

In some embodiments, the CIITA guide RNA comprises a guide sequence of any one of: SEQ ID NOs: 301-302, 320-321, 324, 326-327, 332, 354, 361, 372, 400, 408, 414-415, 419-420, 422, 428, 431-432, 434, 451, 455, 458, 462-464, 468, 504, and 538.

In some embodiments, the CIITA guide RNA comprises a guide sequence of any one of SEQ ID NOs: 301-302, 320, 372, 414, 419, 422, and 462-463.

In some embodiments, the CIITA guide RNA comprises a sequence listed in Table 4. In some embodiments, the CIITA guide RNA comprises a sequence of any one of SEQ ID NOs: 301, 302, 304-576. In some embodiments, the CIITA guide RNA comprises a sequence of SEQ ID NO: 301 or 422. In some embodiments, the CIITA guide RNA comprises a sequence of SEQ ID NO: 301. In some embodiments, the CIITA guide RNA comprises a sequence of SEQ ID NO: 422. In some embodiments, the CIITA guide RNA comprises a guide sequence comprising a sequence of any one of SEQ ID NOs: 301, 302, 304-576. In some embodiments, the CIITA guide RNA comprises a guide sequence comprising a sequence of SEQ ID NO: 301 or 422. In some embodiments, the CIITA guide RNA comprises a guide sequence comprising a sequence of SEQ ID NO: 301. In some embodiments, the CIITA guide RNA comprises a guide sequence comprising a sequence of SEQ ID NO: 422. In some embodiments, the CIITA guide RNA comprises a sequence of any one of SEQ ID NOs: 1301, 1302, 1304-1576. In some embodiments, the CIITA guide RNA comprises a sequence of SEQ ID NO: 1301 or 1422. In some embodiments, the CIITA guide RNA comprises a sequence of SEQ ID NO: 1301. In some embodiments, the CIITA guide RNA comprises a sequence of SEQ ID NO: 1422. In some embodiments, the CIITA guide RNA comprises a sequence of any one of SEQ ID NOs: 2301, 2302, 2304-2576, 3006, and 3007. In some embodiments, the CIITA guide RNA comprises a sequence of any one of SEQ ID NOs: 2301, 2422, 3006, and 3007. In some embodiments, the CIITA guide RNA comprises a sequence of SEQ ID NO: 2301. In some embodiments, the CIITA guide RNA comprises a sequence of SEQ ID NO: 2422. In some embodiments, the CIITA guide RNA comprises a sequence of SEQ ID NO: 3006. In some embodiments, the CIITA guide RNA comprises a sequence of SEQ ID NO: 3007.

In some embodiments, the CIITA guide RNA is a single guide RNA (sgRNA) comprising a sequence of any one of the sgRNA sequences listed in Table 4.

Additional embodiments of CIITA guide RNAs are provided herein, including e.g., exemplary modifications to the guide RNA.

### 1. Genetic Modifications to CIITA

In some embodiments, the methods and compositions disclosed herein genetically modify at least one nucleotide of the CIITA locus in a cell. Because CIITA protein regulates expression of MHC class II, in some embodiments, the genetic modification to CIITA alters the production of CIITA protein, and thereby reduces the expression of MHC class II protein on the surface of the genetically modified cell (or engineered cell). Genetic modifications encompass the population of modifications that results from contact with a genomic editing system (e.g., the population of edits that result from Cas9 and a CIITA guide RNA, or the population of edits that result from BC22 and a CIITA guide RNA).

In some embodiments, the genetic modification comprises at least one nucleotide within the genomic coordinates chosen from: (a) chr16:10877363-10907788 or (b) chr16:10906515-10908136. In some embodiments, the genetic modification comprises at least one nucleotide within the genomic coordinates chosen from any of the genomic coordinates listed in Table 4.

In some embodiments, the genetic modification comprises at least one nucleotide within the genomic coordinates chosen from: chr16:10907504-10907528; chr16:10907508-10907532; chr16:10907539-10907559; chr16:10895658-10895682; chr16:10895668-10895692; chr16:10895750-10895774; chr16:10895753-10895777; chr16:10895754-10895778; chr16:10898684-10898708; chr16:10901529-10901553; chr16:10902121-10902145; chr16:10902701-10902725; chr16:10904726-10904750; chr16:10904760-10904784; chr16:10906493-10906517; chr16:10906515-10906539; chr16:10906631-10906655; chr16:10906636-10906660; chr16:10906643-10906667; chr16:10906770-10906794; chr16:10906788-10906812; chr16:10906789-10906813; chr16:10906816-10906840; chr16:10907148-10907172; chr16:10907254-10907278; chr16:10907331-10907355; chr16:10907477-10907501; chr16:10907497-10907521; chr16:10907503-10907527; and chr16:10907574-10907598.

In some embodiments, the genetic modification comprises at least one nucleotide within the genomic coordinates chosen from: chr16:10895658-10895682; chr16:10902701-10902725; chr16:10906493-10906517; chr16:10906631-10906655; chr16:10906643-10906667; chr16:10907477-10907501; and chr16:10907497-10907521; chr16:10907504-10907528; and chr16:10907508-10907532.

In some embodiments, the genetic modification comprises at least one nucleotide within the genomic coordinates chosen from: chr16:10906889-10906913; and chr16:10907504-10907528.

In some embodiments, the genetic modification comprises at least 5 contiguous nucleotides within the genomic coordinates chosen from: chr16:10907504-10907528; chr16:10907508-10907532; chr16:10907539-10907559; chr16:10895658-10895682; chr16:10895668-10895692; chr16:10895750-10895774; chr16:10895753-10895777; chr16:10895754-10895778; chr16:10898684-10898708; chr16:10901529-10901553; chr16:10902121-10902145; chr16:10902701-10902725; chr16:10904726-10904750; chr16:10904760-10904784; chr16:10906493-10906517; chr16:10906515-10906539; chr16:10906631-10906655; chr16:10906636-10906660; chr16:10906643-10906667; chr16:10906770-10906794; chr16:10906788-10906812; chr16:10906789-10906813; chr16:10906816-10906840; chr16:10907148-10907172; chr16:10907254-10907278; chr16:10907331-10907355; chr16:10907477-10907501; chr16:10907497-10907521; chr16:10907503-10907527; and chr16:10907574-10907598. In some embodiments, the genetic modification comprises at least 10 contiguous nucleotides within the genomic coordinates chosen from: chr16:10907504-10907528; chr16:10907508-10907532; chr16:10907539-10907559; chr16:10895658-10895682; chr16:10895668-10895692; chr16:10895750-10895774; chr16:10895753-10895777; chr16:10895754-10895778; chr16:10898684-10898708; chr16:10901529-10901553; chr16:10902121-10902145; chr16:10902701-10902725; chr16:10904726-10904750; chr16:10904760-10904784; chr16:10906493-10906517; chr16:10906515-10906539; chr16:10906631-10906655; chr16:10906636-10906660; chr16:10906643-10906667; chr16:10906770-10906794; chr16:10906788-10906812; chr16:10906789-10906813; chr16:10906816-10906840; chr16:10907148-10907172; chr16:10907254-10907278; chr16:10907331-10907355; chr16:10907477-10907501; chr16:10907497-10907521; chr16:10907503-10907527; and chr16:10907574-10907598. In some embodiments, the genetic modification comprises at least one indel, at least one C to T substitution, or at least one A to G substitution within the genomic coordinates chosen from: chr16:10907504-10907528; chr16:10907508-10907532; chr16:10907539-10907559; chr16:10895658-10895682; chr16:10895668-10895692; chr16:10895750-10895774; chr16:10895753-10895777; chr16:10895754-10895778; chr16:10898684-10898708; chr16:10901529-10901553; chr16:10902121-10902145; chr16:10902701-10902725; chr16:10904726-10904750; chr16:10904760-10904784; chr16:10906493-10906517; chr16:10906515-10906539; chr16:10906631-10906655; chr16:10906636-10906660; chr16:10906643-10906667; chr16:10906770-10906794; chr16:10906788-10906812; chr16:10906789-10906813; chr16:10906816-10906840; chr16:10907148-10907172; chr16:10907254-10907278; chr16:10907331-10907355; chr16:10907477-10907501; chr16:10907497-10907521; chr16:10907503-10907527; and chr16:10907574-10907598.

In some embodiments, the genetic modification comprises at least 5 contiguous nucleotides within the genomic coordinates chosen from: chr16:10895658-10895682; chr16:10902701-10902725; chr16:10906493-10906517; chr16:10906631-10906655; chr16:10906643-10906667; chr16:10907477-10907501; and chr16:10907497-10907521; chr16:10907504-10907528; and chr16:10907508-10907532. In some embodiments, the genetic modification comprises at least 10 contiguous nucleotides within the genomic coordinates chosen from: chr16:10895658-10895682; chr16:10902701-10902725; chr16:10906493-10906517; chr16:10906631-10906655; chr16:10906643-10906667; chr16:10907477-10907501; and chr16:10907497-10907521; chr16:10907504-10907528; and chr16:10907508-10907532. In some embodiments, the genetic modification comprises at least one indel, at least one C to T substitution, or at least one A to G substitution within the genomic coordinates chosen from: chr16:10895658-10895682; chr16:10902701-10902725; chr16:10906493-10906517; chr16:10906631-10906655; chr16:10906643-10906667; chr16:10907477-10907501; and chr16:10907497-10907521; chr16:10907504-10907528; and chr16:10907508-10907532.

In some embodiments, the genetic modification comprises at least 5 contiguous nucleotides within the genomic coordinates chosen from: chr16:10906889-10906913; and chr16:10907504-10907528. In some embodiments, the genetic modification comprises at least 10 contiguous nucleotides within the genomic coordinates chosen from: chr16:10906889-10906913; and chr16:10907504-10907528. In some embodiments, the genetic modification comprises at least one indel, at least one C to T substitution, or at least one A to G substitution within the genomic coordinates chosen from: chr16:10906889-10906913; and chr16:10907504-10907528.

In some embodiments, the methods and compositions disclosed herein modify the CIITA locus in a cell, wherein the modification to CIITA comprises an insertion of an exogenous nucleic acid. In some embodiments, the exogenous nucleic acid is a protein-coding gene. The protein encoded by the exogenous nucleic acid may be expressed by the cell.

In some embodiments, the modification to CIITA comprises any one or more of an insertion, deletion, substitution or deamination of at least one nucleotide in a target sequence. In some embodiments, the modification to CIITA comprises an insertion of 1, 2, 3, 4 or 5 or more nucleotides in a target sequence. In some embodiments, the modification to CIITA comprises a deletion of 1, 2, 3, 4 or 5 or more nucleotides in a target sequence. In other embodiments, the modification to CIITA comprises an insertion of 1, 2, 3, 4, 5, 6, 7, 8, 9, 10, 15, 20 or 25 or more nucleotides in a target sequence. In other embodiments, the modification to CIITA comprises a deletion of 1, 2, 3, 4, 5, 6, 7, 8, 9, 10, 15, 20 or 25 or more nucleotides in a target sequence. In some embodiments, the modification to CIITA comprises an indel, which is generally defined in the art as an insertion or deletion of less than 1000 base pairs (bp). In some embodiments, the modification to CIITA comprises an indel which results in a frameshift mutation in a target sequence. In some embodiments, the modification to CIITA comprises a substitution of 1, 2, 3, 4, 5, 6, 7, 8, 9, 10, 15, 20 or 25 or more nucleotides in a target sequence. In some embodiments, the modification to CIITA comprises one or more of an insertion, deletion, or substitution of nucleotides resulting from the incorporation of a template nucleic acid. In some embodiments, the modification to CIITA comprises an insertion of a donor nucleic acid in a target sequence. In some embodiments, the modification to CIITA is not transient.

In some embodiments, the methods and compositions disclosed herein modify the CIITA locus in a cell using an RNA-guided DNA binding agent (e.g., a Cas enzyme). In some embodiments, the RNA-guided DNA binding agent is Cas9. In some embodiments, the RNA-guided DNA binding agent cuts within the CIITA gene, wherein the CIITA guide RNA targets a CIITA genomic target sequence comprising at least 10 contiguous nucleotides within the genomic coordinates chosen from: (a) chr16:10877363-10907788 and (b) chr16:10906515-10908136.

In some embodiments, the genetic modification to CIITA results in a truncated form of the CIITA protein. In some embodiments, the truncated CIITA protein does not bind to GTP. In some embodiments, the truncated CIITA protein does not localize to the nucleus. In some embodiments, the CIITA protein (e.g., a truncated form of the CIITA protein) has impaired activity as compared to the wildtype CIITA protein's activity relating to regulating MHC class II expression. In some embodiments, MHC class II expression on the surface of a cell is reduced as a result of impaired CIITA protein activity. In some embodiments, MHC class II expression on the surface of a cell is absent as a result of impaired CIITA protein activity.

### 2. Efficacy of CIITA Guide RNAs

The efficacy of a CIITA guide RNA may be determined by techniques available in the art that assess the editing efficiency of a guide RNA, the levels of CIITA protein or mRNA, or the levels of MHC class II in a target cell. In some embodiments, the reduction or elimination of MHC class II protein on the surface of a cell may be determined by comparison to an unmodified cell (or “relative to an unmodified cell”). An engineered cell or cell population may also be compared to a population of unmodified cells.

In some embodiments, the efficacy of a CIITA guide RNA is determined by measuring levels of CIITA protein in a cell. The levels of CIITA protein may be detected by, e.g., cell lysate and western blot with an anti-CIITA antibody. In some embodiments, the efficacy of a CIITA guide RNA is determined by measuring levels of CIITA protein in the cell nucleus. In some embodiments, the efficacy of a CIITA guide RNA is determined by measuring levels of CIITA mRNA in a cell. The levels of CIITA mRNA may be detected by e.g., RT-PCR. In some embodiments, a decrease in the levels CIITA protein or CIITA mRNA in the target cell as compared to an unmodified cell is indicative of an effective CIITA guide RNA.

An “unmodified cell” (or “unmodified cells”) refers to a control cell (or cells) of the same type of cell in an experiment or test, wherein the “unmodified” control cell has not been contacted with a CIITA guide (i.e., a non-engineered cell). Therefore, an unmodified cell (or cells) may be a cell that has not been contacted with a guide RNA, or a cell that has been contacted with a guide RNA that does not target CIITA.

In some embodiments, the efficacy of a CIITA guide RNA is determined by measuring the reduction or elimination of MHC class II protein expression by the target cells. The CIITA protein functions as a transactivator, activating the MHC class II promoter, and is essential for the expression of MHC class II protein. In some embodiments, MHC class II protein expression may be detected on the surface of the target cells. In some embodiments, MHC class II protein expression is measured by flow cytometry. In some embodiments, an antibody against MHC class II protein (e.g., anti-HLA-DR, -DQ, -DP) may be used to detect MHC class II protein expression e.g., by flow cytometry. In some embodiments, a reduction or elimination in MHC class II protein on the surface of a cell (or population of cells) as compared to an unmodified cell (or population of unmodified cells) is indicative of an effective CIITA guide RNA. In some embodiments, a cell (or population of cells) that has been contacted with a particular CIITA guide RNA and RNA-guided DNA binding agent that is negative for MHC class II protein by flow cytometry is indicative of an effective CIITA guide RNA.

In some embodiments, the MHC class II protein expression is reduced or eliminated in a population of cells using the methods and compositions disclosed herein. In some embodiments, the population of cells is enriched (e.g., by FACS or MACS) and is at least 65%, 70%, 80%, 90%, 91%, 92%, 93%, or 94% MHC class II negative as measured by flow cytometry relative to a population of unmodified cells. In some embodiments, the population of cells is not enriched (e.g., by FACS or MACS) and is at least 65%, 70%, 80%, 90%, 91%, 92%, 93%, or 94% MHC class II negative as measured by flow cytometry relative to a population of unmodified cells.

In some embodiments, the population of cells is at least 65% MHC class II negative as measured by flow cytometry relative to a population of unmodified cells. In some embodiments, the population of cells is at least 70% MHC class II negative as measured by flow cytometry relative to a population of unmodified cells. In some embodiments, the population of cells is at least 80% MHC class II negative as measured by flow cytometry relative to a population of unmodified cells. In some embodiments, the population of cells is at least 90% MHC class II negative as measured by flow cytometry relative to a population of unmodified cells. In some embodiments, the population of cells is at least 91% MHC class II negative as measured by flow cytometry relative to a population of unmodified cells. In some embodiments, the population of cells is at least 92% MHC class II negative as measured by flow cytometry relative to a population of unmodified cells. In some embodiments, the population of cells is at least 93% MHC class II negative as measured by flow cytometry relative to a population of unmodified cells. In some embodiments, the population of cells is at least 94% MHC class II negative as measured by flow cytometry relative to a population of unmodified cells.

In some embodiments, an effective CIITA guide RNA may be determined by measuring the response of immune cells in vitro or in vivo (e.g., CD4+ T cells) to the genetically modified target cell. A CD4+ T cell response may be evaluated by an assay that measures the activation response of CD4+ T cells e.g., CD4+ T cell proliferation, expression of activation markers, or cytokine production (IL-2, IL-12, IFN-7) (e.g., flow cytometry, ELISA). The response of CD4+ T cells may be evaluated in in vitro cell culture assays in which the genetically modified cell is co-cultured with cells comprising CD4+ T cells. For example, the genetically modified cell may be co-cultured e.g., with PBMCs, purified CD3+ T cells comprising CD4+ T cells, purified CD4+ T cells, or a CD4+ T cell line. The CD4+ T cell response elicited from the genetically modified cell may be compared to the response elicited from an unmodified cell. A reduced response from CD4+ T cells is indicative of an effective CIITA guide RNA.

The efficacy of a CIITA guide RNA may also be assessed by the survival of the cell post-editing. In some embodiments, the cell survives post editing for at least one week to six weeks. In some embodiments, the cell survives post editing for at least one week to twelve weeks. In some embodiments, the cell survives post editing for at least two weeks. In some embodiments, the cell survives post editing for at least three weeks. In some embodiments, the cell survives post editing for at least four weeks. In some embodiments, the cell survives post editing for at least five weeks. In some embodiments, the cell survives post editing for at least six weeks. The viability of a genetically modified cell may be measured using standard techniques, including e.g., by measures of cell death, by flow cytometry live/dead staining, or cell proliferation.

### G. AAVS1 Guide RNAs

The methods and compositions provided herein disclose AAVS1 guide RNAs useful for creating insertion sites within the AAVS1 locus. In some embodiments, such guide RNAs direct an RNA-guided DNA binding agent to am AAVS1 genomic target sequence and may be referred to herein as “AAVS1 guide RNAs.” In some embodiments, the AAVS1 guide RNA directs an RNA-guided DNA binding agent to a human AAVS1 genomic target sequence. In some embodiments, the AAVS1 guide RNA comprises a guide sequence selected from SEQ ID NO: 601-774.

In some embodiments, the methods and compositions disclosed herein comprise an AAVS1 guide RNA comprising a guide sequence that targets an AAVS1 genomic target sequence comprising at least 10 contiguous nucleotides within the genomic coordinates chr19: 55115151-55116209. In some embodiments, the methods and compositions disclosed herein comprise a AAVS1 guide RNA comprising a guide sequence that targets an AAVS1 genomic target sequence comprising at least one nucleotide within the genomic coordinates chr19: 55115151-55116209.

In some embodiments, the methods and compositions disclosed herein comprise a AAVS1 guide RNA comprising a guide sequence that directs an RNA-guided DNA binding agent to make cut in an AAVS1 gene, wherein the AAVS1 guide RNA targets an AAVS1 genomic target sequence comprising at least 10 contiguous nucleotides within the genomic coordinates chr19: 55115151-55116209. In some embodiments, the methods and compositions disclosed herein comprise an AAVS1 guide RNA comprising a guide sequence that directs an RNA-guided DNA binding agent to make cut in an AAVS1 gene, wherein the AAVS1 guide RNA targets an AAVS1 genomic target sequence comprising at least one nucleotide within the genomic coordinates chr19: 55115151-55116209.

In some embodiments, the methods and compositions disclose an AAVS1 guide RNA that directs an RNA-guided DNA binding agent to induce a double stranded break (DSB) or a single-stranded break (SSB) in an AAVS1 genomic target sequence. In some embodiments, the methods and compositions disclose an AAVS1 guide RNA that directs an RNA-guided DNA binding agent to make a cut in an AAVS1 genomic target sequence. In embodiments wherein the RNA-guided DNA cutting agent is Cas9, the cut or “cut site” occurs at the third base from the protospacer adjacent motif (PAM) sequence.

In some embodiments, a composition is provided comprising an AAVS1 guide RNA described herein and an RNA-guided DNA binding agent or a nucleic acid encoding an RNA-guided DNA binding agent.

In some embodiments, a composition is provided comprising an AAVS1 single-guide RNA (sgRNA) comprising a guide sequence that targets a genomic target comprising at least 10 contiguous nucleotides within the genomic coordinates chr19: 55115151-55116209. In some embodiments, a composition is provided comprising an AAVS1 sgRNA described herein and an RNA-guided DNA binding agent or a nucleic acid encoding an RNA-guided DNA binding agent.

In some embodiments, a composition is provided comprising an AAVS1 dual-guide RNA (dgRNA) comprising a guide sequence that targets a genomic target comprising at least 10 contiguous nucleotides within the genomic coordinates chr19: 55115151-55116209. In some embodiments, a composition is provided comprising an AAVS1 dgRNA described herein and an RNA-guided DNA binding agent or a nucleic acid encoding an RNA-guided DNA binding agent.

In some embodiments, the AAVS1 gRNA comprises a guide sequence selected from any one of SEQ ID NOs: 601-774. Exemplary AAVS1 guide sequences are shown below in Table 5.

TABLE 5
Exemplary AAVS1 guide sequences.

| | | | Exemplary | | |
|---|---|---|---|---|---|
| | SEQ ID | | Guide RNA | Exemplary Guide | |
| | NO to | | Full Sequence | RNA Modified | |
| Guide | the Guide | Guide | (SEQ ID NOs: | Sequence (SEQ ID | Genomic |
| ID | Sequence | Sequence | 1601-1774) | Nos: 2601-2774) | Coordinates (hg38) |
| G025808 | 601 | AUAUCCAGAAC | AUAUCCAGAAC | mA*mU*mA*mUmCCAm | chr14: 22547505- |
| | | CCUGACCCUGC | CCUGACCCUGC | GmAAmCCmCUGACmCC | 22547529 |
| | | CG | CGGUUGUAGCU | UGmCCGmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025809 | 602 | CCCACAGAUAU | CCCACAGAUAU | mC*mC*mC*mAmCAGm | chr14: 22547498- |
| | | CCAGAACCCUG | CCAGAACCCUG | AmUAmUCmCAGAAmCC | 22547522 |
| | | AC | ACGUUGUAGCU | CUmGACmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025810 | 603 | UUGUCCCACAG | UUGUCCCACAG | mU*mU*mG*mUmCCCm | chr14: 22547494- |
| | | AUAUCCAGAAC | AUAUCCAGAAC | AmCAmGAmUAUCCmAG | 22547518 |
| | | CC | CCGUUGUAGCU | AAmCCCmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025811 | 604 | CUUGUCCCACA | CUUGUCCCACA | mC*mU*mU*mGmUCCm | chr14: 22547493- |
| | | GAUAUCCAGAA | GAUAUCCAGAA | CmACmAGmAUAUCmCA | 22547517 |
| | | CC | CCGUUGUAGCU | GAmACCmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025812 | 605 | AUCCUCUUGUC | AUCCUCUUGUC | mA*mU*mC*mCmUCUm | chr14: 22547488- |
| | | CCACAGAUAUC | CCACAGAUAUC | UmGUmCCmCACAGmAU | 22547512 |
| | | CA | CAGUUGUAGCU | AUmCCAmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025813 | 606 | GAUCCUCUUGU | GAUCCUCUUGU | mG*mA*mU*mCmCUCm | chr14: 22547487- |
| | | CCCACAGAUAU | CCCACAGAUAU | UmUGmUCmCCACAmGA | 22547511 |
| | | CC | CCGUUGUAGCU | UAmUCCmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025814 | 607 | AACCCUGAUCC | AACCCUGAUCC | mA*mA*mC*mCmCUGm | chr14: 22547481- |
| | | UCUUGUCCCAC | UCUUGUCCCAC | AmUCmCUmCUUGUmCC | 22547505 |
| | | AG | AGGUUGUAGCU | CAmCAGmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025815 | 608 | GCCGUGUACCA | GCCGUGUACCA | mG*mC*mC*mGmUGUm | chr14: 22547525- |
| | | GCUGAGAGACU | GCUGAGAGACU | AmCCmAGmCUGAGmAG | 22547549 |
| | | CU | CUGUUGUAGCU | ACmUCUmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025816* | 609 | AAGAGAAAGGG | AAGAGAAAGGG | mA*mA*mG*mAmGAAm | chr19: 55115151- |
| | | AGUAGAGGCGG | AGUAGAGGCGG | AmGGmGAmGUAGAmGG | 55115175 |
| | | CC | CCGUUGUAGCU | CGmGCCmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025817 | 610 | AGUAGAGGCGG | AGUAGAGGCGG | mA*mG*mU*mAmGAGm | chr19: 55115162- |
| | | CCACGACCUGG | CCACGACCUGG | GmCGmGCmCACGAmCC | 55115186 |
| | | UG | UGGUUGUAGCU | UGmGUGmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025818 | 611 | CCACGACCUGG | CCACGACCUGG | mC*mC*mA*mCmGACm | chr19: 55115173- |
| | | UGAACACCUAG | UGAACACCUAG | CmUGmGUmGAACAmCC | 55115197 |
| | | GA | GAGUUGUAGCU | UAmGGAmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025819 | 612 | GACGCACCAUU | GACGCACCAUU | mG*mA*mC*mGmCACm | chr19: 55115195- |
| | | CUCACAAAGGG | CUCACAAAGGG | CmAUmUCmUCACAmAA | 55115219 |
| | | AG | AGGUUGUAGCU | GGmGAGmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025820 | 613 | CUCACAAAGGG | CUCACAAAGGG | mC*mU*mC*mAmCAAm | chr19: 55115206- |
| | | AGUUUUCCACA | AGUUUUCCACA | AmGGmGAmGUUUUmCC | 55115230 |
| | | CG | CGGUUGUAGCU | ACmACGmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025821 | 614 | UCACAAAGGGA | UCACAAAGGGA | mU*mC*mA*mCmAAAm | chr19: 55115207- |
| | | GUUUUCCACAC | GUUUUCCACAC | GmGGmAGmUUUUCmCA | 55115231 |
| | | GG | GGGUUGUAGCU | CAmCGGmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025822 | 615 | CACAAAGGGAG | CACAAAGGGAG | mC*mA*mC*mAmAAGm | chr19: 55115208- |
| | | UUUUCCACACG | UUUUCCACACG | GmGAmGUmUUUCCmAC | 55115232 |
| | | GA | GAGUUGUAGCU | ACmGGAmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025823 | 616 | ACAAAGGGAGU | ACAAAGGGAGU | mA*mC*mA*mAmAGGm | chr19: 55115209- |
| | | UUUCCACACGG | UUUCCACACGG | GmAGmUUmUUCCAmCA | 55115233 |
| | | AC | ACGUUGUAGCU | CGmGACmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025824 | 617 | CAAAGGGAGUU | CAAAGGGAGUU | mC*mA*mA*mAmGGGm | chr19: 55115210- |
| | | UUCCACACGGA | UUCCACACGGA | AmGUmUUmUCCACmAC | 55115234 |
| | | CA | CAGUUGUAGCU | GGmACAmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025825 | 618 | AGGGAGUUUUC | AGGGAGUUUUC | mA*mG*mG*mGmAGUm | chr19: 55115213- |
| | | CACACGGACAC | CACACGGACAC | UmUUmCCmACACGmGA | 55115237 |
| | | CC | CCGUUGUAGCU | CAmCCCmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025826 | 619 | GUUUUCCACAC | GUUUUCCACAC | mG*mU*mU*mUmUCCm | chr19: 55115218- |
| | | GGACACCCCCC | GGACACCCCCC | AmCAmCGmGACACmCC | 55115242 |
| | | UC | UCGUUGUAGCU | CCmCUCmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025827 | 620 | CACACGGACAC | CACACGGACAC | mC*mA*mC*mAmCGGm | chr19: 55115224- |
| | | CCCCCUCCUCA | CCCCCUCCUCA | AmCAmCCmCCCCUmCC | 55115248 |
| | | CC | CCGUUGUAGCU | UCmACCmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025828 | 621 | ACACGGACACC | ACACGGACACC | mA*mC*mA*mCmGGAm | chr19: 55115225- |
| | | CCCCUCCUCAC | CCCCUCCUCAC | CmACmCCmCCCUCmCU | 55115249 |
| | | CA | CAGUUGUAGCU | CAmCCAmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025829 | 622 | GGACACCCCCC | GGACACCCCCC | mG*mG*mA*mCmACCm | chr19: 55115229- |
| | | UCCUCACCACA | UCCUCACCACA | CmCCmCUmCCUCAmCC | 55115253 |
| | | GC | GCGUUGUAGCU | ACmAGCmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025830 | 623 | GCCCUGCCAGG | GCCCUGCCAGG | mG*mC*mC*mCmUGCm | chr19: 55115251- |
| | | ACGGGGCUGGC | ACGGGGCUGGC | CmAGmGAmCGGGGmCU | 55115275 |
| | | UA | UAGUUGUAGCU | GGmCUAmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025831 | 624 | GGUGAAGAGCC | GGUGAAGAGCC | mG*mG*mU*mGmAAGm | chr19: 55115337- |
| | | AAAGUUAGAAC | AAAGUUAGAAC | AmGCmCAmAAGUUmAG | 55115361 |
| | | UC | UCGUUGUAGCU | AAmCUCmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025832* | 625 | UCUUGGGAAGU | UCUUGGGAAGU | mU*mC*mU*mUmGGGm | chr19: 55115404- |
| | | GUAAGGAAGCU | GUAAGGAAGCU | AmAGmUGmUAAGGmAA | 55115428 |
| | | GC | GCGUUGUAGCU | GCmUGCmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025833 | 626 | GCUGCAGCACC | GCUGCAGCACC | mG*mC*mU*mGmCAGm | chr19: 55115423- |
| | | AGGAUCAGUGA | AGGAUCAGUGA | CmACmCAmGGAUCmAG | 55115447 |
| | | AA | AAGUUGUAGCU | UGmAAAmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025834 | 627 | ACCAGGAUCAG | ACCAGGAUCAG | mA*mC*mC*mAmGGAm | chr19: 55115431- |
| | | UGAAACGCACC | UGAAACGCACC | UmCAmGUmGAAACmGC | 55115455 |
| | | AG | AGGUUGUAGCU | ACmCAGmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025835 | 628 | GGUUCUGGGAG | GGUUCUGGGAG | mG*mG*mU*mUmCUGm | chr19: 55115477- |
| | | AGGGUAGCGCA | AGGGUAGCGCA | GmGAmGAmGGGUAmGC | 55115501 |
| | | GG | GGGUUGUAGCU | GCmAGGmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025836 | 629 | GAGGGUAGCGC | GAGGGUAGCGC | mG*mA*mG*mGmGUAm | chr19: 55115487- |
| | | AGGGUGGCCAC | AGGGUGGCCAC | GmCGmCAmGGGUGmGC | 55115511 |
| | | UG | UGGUUGUAGCU | CAmCUGmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025837 | 630 | GCCACUGAGAA | GCCACUGAGAA | mG*mC*mC*mAmCUGm | chr19: 55115504- |
| | | CCGGGCAGGUC | CCGGGCAGGUC | AmGAmACmCGGGCmAG | 55115528 |
| | | AC | ACGUUGUAGCU | GUmCACmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025838 | 631 | CCACUGAGAAC | CCACUGAGAAC | mC*mC*mA*mCmUGAm | chr19: 55115505- |
| | | CGGGCAGGUCA | CGGGCAGGUCA | GmAAmCCmGGGCAmGG | 55115529 |
| | | CG | CGGUUGUAGCU | UCmACGmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025839 | 632 | CACUGAGAACC | CACUGAGAACC | mC*mA*mC*mUmGAGm | chr19: 55115506- |
| | | GGGCAGGUCAC | GGGCAGGUCAC | AmACmCGmGGCAGmGU | 55115530 |
| | | GC | GCGUUGUAGCU | CAmCGCmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025840* | 633 | ACUGAGAACCG | ACUGAGAACCG | mA*mC*mU*mGmAGAm | chr19: 55115507- |
| | | GGCAGGUCACG | GGCAGGUCACG | AmCCmGGmGCAGGmUC | 55115531 |
| | | CA | CAGUUGUAGCU | ACmGCAmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025841* | 634 | AACCGGGCAGG | AACCGGGCAGG | mA*mA*mC*mCmGGGm | chr19: 55115513- |
| | | UCACGCAUCCC | UCACGCAUCCC | CmAGmGUmCACGCmAU | 55115537 |
| | | CC | CCGUUGUAGCU | CCmCCCmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025842 | 635 | ACCGGGCAGGU | ACCGGGCAGGU | mA*mC*mC*mGmGGCm | chr19: 55115514- |
| | | CACGCAUCCCC | CACGCAUCCCC | AmGGmUCmACGCAmUC | 55115538 |
| | | CC | CCGUUGUAGCU | CCmCCCmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025843 | 636 | GGGCAGGUCAC | GGGCAGGUCAC | mG*mG*mG*mCmAGGm | chr19: 55115517- |
| | | GCAUCCCCCCC | GCAUCCCCCCC | UmCAmCGmCAUCCmCC | 55115541 |
| | | UU | UUGUUGUAGCU | CCmCUUmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025844 | 637 | GGCAGGUCACG | GGCAGGUCACG | mG*mG*mC*mAmGGUm | chr19: 55115518- |
| | | CAUCCCCCCCU | CAUCCCCCCCU | CmACmGCmAUCCCmCC | 55115542 |
| | | UC | UCGUUGUAGCU | CCmUUCmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025845 | 638 | CCUUCCCUCCC | CCUUCCCUCCC | mC*mC*mU*mUmCCCm | chr19: 55115537- |
| | | ACCCCCUGCCA | ACCCCCUGCCA | UmCCmCAmCCCCCmUG | 55115561 |
| | | AG | AGGUUGUAGCU | CCmAAGmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025846 | 639 | CUUCCCUCCCA | CUUCCCUCCCA | mC*mU*mU*mCmCCUm | chr19: 55115538- |
| | | CCCCCUGCCAA | CCCCCUGCCAA | CmCCmACmCCCCUmGC | 55115562 |
| | | GC | GCGUUGUAGCU | CAmAGCmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025847 | 640 | CCCCCUGCCAA | CCCCCUGCCAA | mC*mC*mC*mCmCUGm | chr19: 55115549- |
| | | GCUCUCCCUCC | GCUCUCCCUCC | CmCAmAGmCUCUCmCC | 55115573 |
| | | CA | CAGUUGUAGCU | UCmCCAmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025848 | 641 | GAUCCUCUCUG | GAUCCUCUCUG | mG*mA*mU*mCmCUCm | chr19: 55115574- |
| | | GCUCCAUCGUA | GCUCCAUCGUA | UmCUmGGmCUCCAmUC | 55115598 |
| | | AG | AGGUUGUAGCU | GUmAAGmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025849 | 642 | AGAGGUUCUGG | AGAGGUUCUGG | mA*mG*mA*mGmGUUm | chr19: 55115606- |
| | | CAAGGAGAGAG | CAAGGAGAGAG | CmUGmGCmAAGGAmGA | 55115630 |
| | | AU | AUGUUGUAGCU | GAmGAUmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025850 | 643 | GGGGGUGUGUC | GGGGGUGUGUC | mG*mG*mG*mGmGUGm | chr19: 55115643- |
| | | ACCAGAUAAGG | ACCAGAUAAGG | UmGUmCAmCCAGAmUA | 55115667 |
| | | AA | AAGUUGUAGCU | AGmGAAmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025851 | 644 | CAAUAUCAGGA | CAAUAUCAGGA | mC*mA*mA*mUmAUCm | chr19: 55115696- |
| | | GACUAGGAAGG | GACUAGGAAGG | AmGGmAGmACUAGmGA | 55115720 |
| | | AG | AGGUUGUAGCU | AGmGAGmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025852 | 645 | AUGGGGCUUUU | AUGGGGCUUUU | mA*mU*mG*mGmGGCm | chr19: 55115731- |
| | | CUGUCACCAAU | CUGUCACCAAU | UmUUmUCmUGUCAmCC | 55115755 |
| | | CC | CCGUUGUAGCU | AAmUCCmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025853 | 646 | UUUCUGUCACC | UUUCUGUCACC | mU*mU*mU*mCmUGUm | chr19: 55115739- |
| | | AAUCCUGUCCC | AAUCCUGUCCC | CmACmCAmAUCCUmGU | 55115763 |
| | | UA | UAGUUGUAGCU | CCmCUAmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025854 | 647 | UUCUGUCACCA | UUCUGUCACCA | mU*mU*mC*mUmGUCm | chr19: 55115740- |
| | | AUCCUGUCCCU | AUCCUGUCCCU | AmCCmAAmUCCUGmUC | 55115764 |
| | | AG | AGGUUGUAGCU | CCmUAGmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025855 | 648 | UCUGUCACCAA | UCUGUCACCAA | mU*mC*mU*mGmUCAm | chr19: 55115741- |
| | | UCCUGUCCCUA | UCCUGUCCCUA | CmCAmAUmCCUGUmCC | 55115765 |
| | | GU | GUGUUGUAGCU | CUmAGUmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025856 | 649 | GUGGGGUGGAG | GUGGGGUGGAG | mG*mU*mG*mGmGGUm | chr19: 55115774- |
| | | GGGACAGAUAA | GGGACAGAUAA | GmGAmGGmGGACAmGA | 55115798 |
| | | AA | AAGUUGUAGCU | UAmAAAmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025857 | 650 | UGGAGGGGACA | UGGAGGGGACA | mU*mG*mG*mAmGGGm | chr19 : 55115780- |
| | | GAUAAAAGUAC | GAUAAAAGUAC | GmACmAGmAUAAAmAG | 55115804 |
| | | CC | CCGUUGUAGCU | UAmCCCmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025858 | 651 | GGACAGAUAAA | GGACAGAUAAA | mG*mG*mA*mCmAGAm | chr19: 55115786- |
| | | AGUACCCAGAA | AGUACCCAGAA | UmAAmAAmGUACCmCA | 55115810 |
| | | CC | CCGUUGUAGCU | GAmACCmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025859 | 652 | AAAGUACCCAG | AAAGUACCCAG | mA*mA*mA*mGmUACm | chr19: 55115795- |
| | | AACCAGAGCCA | AACCAGAGCCA | CmCAmGAmACCAGmAG | 55115819 |
| | | CA | CAGUUGUAGCU | CCmACAmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025860 | 653 | UACCCAGAACC | UACCCAGAACC | mU*mA*mC*mCmCAGm | chr19: 55115799- |
| | | AGAGCCACAUU | AGAGCCACAUU | AmACmCAmGAGCCmAC | 55115823 |
| | | AA | AAGUUGUAGCU | AUmUAAmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025861 | 654 | ACCCAGAACCA | ACCCAGAACCA | mA*mC*mC*mCmAGAm | chr19: 55115800- |
| | | GAGCCACAUUA | GAGCCACAUUA | AmCCmAGmAGCCAmCA | 55115824 |
| | | AC | ACGUUGUAGCU | UUmAACmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025862 | 655 | UUAACCGGCCC | UUAACCGGCCC | mU*mU*mA*mAmCCGm | chr19: 55115819- |
| | | UGGGAAUAUAA | UGGGAAUAUAA | GmCCmCUmGGGAAmUA | 55115843 |
| | | GG | GGGUUGUAGCU | UAmAGGmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025863 | 656 | UAACCGGCCCU | UAACCGGCCCU | mU*mA*mA*mCmCGGm | chr19: 55115820- |
| | | GGGAAUAUAAG | GGGAAUAUAAG | CmCCmUGmGGAAUmAU | 55115844 |
| | | GU | GUGUUGUAGCU | AAmGGUmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025864 | 657 | AGGUGGUCCCA | AGGUGGUCCCA | mA*mG*mG*mUmGGUm | chr19: 55115840- |
| | | GCUCGGGGACA | GCUCGGGGACA | CmCCmAGmCUCGGmGG | 55115864 |
| | | CA | CAGUUGUAGCU | ACmACAmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025865 | 658 | GGAUCCCUGGA | GGAUCCCUGGA | mG*mG*mA*mUmCCCm | chr19: 55115864- |
| | | GGCAGCAAACA | GGCAGCAAACA | UmGGmAGmGCAGCmAA | 55115888 |
| | | UG | UGGUUGUAGCU | ACmAUGmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025866 | 659 | ACAUGCUGUCC | ACAUGCUGUCC | mA*mC*mA*mUmGCUm | chr19: 55115883- |
| | | UGAAGUGGACA | UGAAGUGGACA | GmUCmCUmGAAGUmGG | 55115907 |
| | | UA | UAGUUGUAGCU | ACmAUAmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025867 | 660 | CAUGCUGUCCU | CAUGCUGUCCU | mC*mA*mU*mGmCUGm | chr19: 55115884- |
| | | GAAGUGGACAU | GAAGUGGACAU | UmCCmUGmAAGUGmGA | 55115908 |
| | | AG | AGGUUGUAGCU | CAmUAGmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025868 | 661 | GAGGAAGAAGA | GAGGAAGAAGA | mG*mA*mG*mGmAAGm | chr19: 55115920- |
| | | CUAGCUGAGCU | CUAGCUGAGCU | AmAGmACmUAGCUmGA | 55115944 |
| | | CU | CUGUUGUAGCU | GCmUCUmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025869 | 662 | AGGAAGAAGAC | AGGAAGAAGAC | mA*mG*mG*mAmAGAm | chr19: 55115921- |
| | | UAGCUGAGCUC | UAGCUGAGCUC | AmGAmCUmAGCUGmAG | 55115945 |
| | | UC | UCGUUGUAGCU | CUmCUCmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025870 | 663 | GGAAGAAGACU | GGAAGAAGACU | mG*mG*mA*mAmGAAm | chr19: 55115922- |
| | | AGCUGAGCUCU | AGCUGAGCUCU | GmACmUAmGCUGAmGC | 55115946 |
| | | CG | CGGUUGUAGCU | UCmUCGmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025871 | 664 | AGCUGAGCUCU | AGCUGAGCUCU | mA*mG*mC*mUmGAGm | chr19: 55115933- |
| | | CGGACCCCUGG | CGGACCCCUGG | CmUCmUCmGGACCmCC | 55115957 |
| | | AA | AAGUUGUAGCU | UGmGAAmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025872 | 665 | CUGCCCAAAUG | CUGCCCAAAUG | mC*mU*mG*mCmCCAm | chr19: 55116019- |
| | | AAAGGAGUGAG | AAAGGAGUGAG | AmAUmGAmAAGGAmGU | 55116043 |
| | | AG | AGGUUGUAGCU | GAmGAGmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025873 | 666 | UGCCCAAAUGA | UGCCCAAAUGA | mU*mG*mC*mCmCAAm | chr19: 55116020- |
| | | AAGGAGUGAGA | AAGGAGUGAGA | AmUGmAAmAGGAGmUG | 55116044 |
| | | GG | GGGUUGUAGCU | AGmAGGmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025874 | 667 | AAUGAAAGGAG | AAUGAAAGGAG | mA*mA*mU*mGmAAAm | chr19: 55116026- |
| | | UGAGAGGUGAC | UGAGAGGUGAC | GmGAmGUmGAGAGmGU | 55116050 |
| | | CC | CCGUUGUAGCU | GAmCCCmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025875 | 668 | GACCCGAAUCC | GACCCGAAUCC | mG*mA*mC*mCmCGAm | chr19: 55116045- |
| | | ACAGGAGAACG | ACAGGAGAACG | AmUCmCAmCAGGAmGA | 55116069 |
| | | GG | GGGUUGUAGCU | ACmGGGmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025876* | 669 | AAGCAAGAGGA | AAGCAAGAGGA | mA*mA*mG*mCmAAGm | chr19: 55116084- |
| | | UGGAGAGGUGG | UGGAGAGGUGG | AmGGmAUmGGAGAmGG | 55116108 |
| | | CU | CUGUUGUAGCU | UGmGCUmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025877 | 670 | UUGUCCAGAAA | UUGUCCAGAAA | mU*mU*mG*mUmCCAm | chr19: 55116136- |
| | | AACGGUGAUGA | AACGGUGAUGA | GmAAmAAmACGGUmGA | 55116160 |
| | | UG | UGGUUGUAGCU | UGmAUGmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025878 | 671 | UGGCCGCCUCU | UGGCCGCCUCU | mU*mG*mG*mCmCGCm | chr19: 55115152- |
| | | ACUCCCUUUCU | ACUCCCUUUCU | CmUCmUAmCUCCCmUU | 55115176 |
| | | CU | CUGUUGUAGCU | UCmUCUmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025879 | 672 | GUUCACCAGGU | GUUCACCAGGU | mG*mU*mU*mCmACCm | chr19: 55115165- |
| | | CGUGGCCGCCU | CGUGGCCGCCU | AmGGmUCmGUGGCmCG | 55115189 |
| | | CU | CUGUUGUAGCU | CCmUCUmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025880 | 673 | UGUUCACCAGG | UGUUCACCAGG | mU*mG*mU*mUmCACm | chr19: 55115166- |
| | | UCGUGGCCGCC | UCGUGGCCGCC | CmAGmGUmCGUGGmCC | 55115190 |
| | | UC | UCGUUGUAGCU | GCmCUCmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025881 | 674 | GUCCUAGGUGU | GUCCUAGGUGU | mG*mU*mC*mCmUAGm | chr19: 55115174- |
| | | UCACCAGGUCG | UCACCAGGUCG | GmUGmUUmCACCAmGG | 55115198 |
| | | UG | UGGUUGUAGCU | UCmGUGmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025882 | 675 | UGCGUCCUAGG | UGCGUCCUAGG | mU*mG*mC*mGmUCCm | chr19: 55115177- |
| | | UGUUCACCAGG | UGUUCACCAGG | UmAGmGUmGUUCAmCC | 55115201 |
| | | UC | UCGUUGUAGCU | AGmGUCmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025883 | 676 | UGUGAGAAUGG | UGUGAGAAUGG | mU*mG*mU*mGmAGAm | chr19: 55115188- |
| | | UGCGUCCUAGG | UGCGUCCUAGG | AmUGmGUmGCGUCmCU | 55115212 |
| | | UG | UGGUUGUAGCU | AGmGUGmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025884 | 677 | GAAAACUCCCU | GAAAACUCCCU | mG*mA*mA*mAmACUm | chr19: 55115200- |
| | | UUGUGAGAAUG | UUGUGAGAAUG | CmCCmUUmUGUGAmGA | 55115224 |
| | | GU | GUGUUGUAGCU | AUmGGUmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025885 | 678 | ACCUGUGAGAU | ACCUGUGAGAU | mA*mC*mC*mUmGUGm | chr19: 55115270- |
| | | AAGGCCAGUAG | AAGGCCAGUAG | AmGAmUAmAGGCCmAG | 55115294 |
| | | CC | CCGUUGUAGCU | UAmGCCmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025886 | 679 | UACCUGUGAGA | UACCUGUGAGA | mU*mA*mC*mCmUGUm | chr19: 55115271- |
| | | UAAGGCCAGUA | UAAGGCCAGUA | GmAGmAUmAAGGCmCA | 55115295 |
| | | GC | GCGUUGUAGCU | GUmAGCmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025887 | 680 | UUACCUGUGAG | UUACCUGUGAG | mU*mU*mA*mCmCUGm | chr19: 55115272- |
| | | AUAAGGCCAGU | AUAAGGCCAGU | UmGAmGAmUAAGGmCC | 55115296 |
| | | AG | AGGUUGUAGCU | AGmUAGmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025888* | 681 | AGUUUUACCUG | AGUUUUACCUG | mA*mG*mU*mUmUUAm | chr19: 55115276- |
| | | UGAGAUAAGGC | UGAGAUAAGGC | CmCUmGUmGAGAUmAA | 55115300 |
| | | CA | CAGUUGUAGCU | GGmCCAmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025889 | 682 | GUGCGUCAGUU | GUGCGUCAGUU | mG*mU*mG*mCmGUCm | chr19: 55115283- |
| | | UUACCUGUGAG | UUACCUGUGAG | AmGUmUUmUACCUmGU | 55115307 |
| | | AU | AUGUUGUAGCU | GAmGAUmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025890 | 683 | UAUAUUGUUCC | UAUAUUGUUCC | mU*mA*mU*mAmUUGm | chr19: 55115297- |
| | | UCCGUGCGUCA | UCCGUGCGUCA | UmUCmCUmCCGUGmCG | 55115321 |
| | | GU | GUGUUGUAGCU | UCmAGUmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025891 | 684 | UAACUUUGGCU | UAACUUUGGCU | mU*mA*mA*mCmUUUm | chr19: 55115331- |
| | | CUUCACCUUUC | CUUCACCUUUC | GmGCmUCmUUCACmCU | 55115355 |
| | | UA | UAGUUGUAGCU | UUmCUAmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025892 | 685 | CUAACUUUGGC | CUAACUUUGGC | mC*mU*mA*mAmCUUm | chr19: 55115332- |
| | | UCUUCACCUUU | UCUUCACCUUU | UmGGmCUmCUUCAmCC | 55115356 |
| | | CU | CUGUUGUAGCU | UUmUCUmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025893 | 686 | UCUAACUUUGG | UCUAACUUUGG | mU*mC*mU*mAmACUm | chr19: 55115333- |
| | | CUCUUCACCUU | CUCUUCACCUU | UmUGmGCmUCUUCmAC | 55115357 |
| | | UC | UCGUUGUAGCU | CUmUUCmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025894 | 687 | GGUCCUGAGUU | GGUCCUGAGUU | mG*mG*mU*mCmCUGm | chr19: 55115343- |
| | | CUAACUUUGGC | CUAACUUUGGC | AmGUmUCmUAACUmUU | 55115367 |
| | | UC | UCGUUGUAGCU | GGmCUCmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025895 | 688 | UCCUGGUGCUG | UCCUGGUGCUG | mU*mC*mC*mUmGGUm | chr19: 55115414- |
| | | CAGCUUCCUUA | CAGCUUCCUUA | GmCUmGCmAGCUUmCC | 55115438 |
| | | CA | CAGUUGUAGCU | UUmACAmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G021566 | 689 | CAGCUAGUCUU | CAGCUAGUCUU | mC*mA*mG*mCmUAGm | chr19: 55115914- |
| | | CUUCCUCCAAC | CUUCCUCCAAC | UmCUmUCmUUCCUmCC | 55115938 |
| | | CC | CCGUUGUAGCU | AAmCCCmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025896 | 690 | GCGUGACCUGC | GCGUGACCUGC | mG*mC*mG*mUmGACm | chr19: 55115506- |
| | | CCGGUUCUCAG | CCGGUUCUCAG | CmUGmCCmCGGUUmCU | 55115530 |
| | | UG | UGGUUGUAGCU | CAmGUGmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025897 | 691 | GAUGCGUGACC | GAUGCGUGACC | mG*mA*mU*mGmCGUm | chr19: 55115509- |
| | | UGCCCGGUUCU | UGCCCGGUUCU | GmACmCUmGCCCGmGU | 55115533 |
| | | CA | CAGUUGUAGCU | UCmUCAmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025898 | 692 | GUUUGCUUACG | GUUUGCUUACG | mG*mU*mU*mUmGCUm | chr19: 55115579- |
| | | AUGGAGCCAGA | AUGGAGCCAGA | UmACmGAmUGGAGmCC | 55115603 |
| | | GA | GAGUUGUAGCU | AGmAGAmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025899 | 693 | GAACCUCUAAG | GAACCUCUAAG | mG*mA*mA*mCmCUCm | chr19: 55115590- |
| | | GUUUGCUUACG | GUUUGCUUACG | UmAAmGGmUUUGCmUU | 55115614 |
| | | AU | AUGUUGUAGCU | ACmGAUmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025900* | 694 | CCUGGAGCCAU | CCUGGAGCCAU | mC*mC*mU*mGmGAGm | chr19: 55115615- |
| | | CUCUCUCCUUG | CUCUCUCCUUG | CmCAmUCmUCUCUmCC | 55115639 |
| | | CC | CCGUUGUAGCU | UUmGCCmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025901 | 695 | CACCCCCAUUU | CACCCCCAUUU | mC*mA*mC*mCmCCCm | chr19: 55115626- |
| | | CCUGGAGCCAU | CCUGGAGCCAU | AmUUmUCmCUGGAmGC | 55115650 |
| | | CU | CUGUUGUAGCU | CAmUCUmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025902* | 696 | UCUGGUGACAC | UCUGGUGACAC | mU*mC*mU*mGmGUGm | chr19: 55115636- |
| | | ACCCCCAUUUC | ACCCCCAUUUC | AmCAmCAmCCCCCmAU | 55115660 |
| | | CU | CUGUUGUAGCU | UUmCCUmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025903 | 697 | UUCCUUAUCUG | UUCCUUAUCUG | mU*mU*mC*mCmUUAm | chr19: 55115643- |
| | | GUGACACACCC | GUGACACACCC | UmCUmGGmUGACAmCA | 55115667 |
| | | CC | CCGUUGUAGCU | CCmCCCmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025904 | 698 | GGCAGAUUCCU | GGCAGAUUCCU | mG*mG*mC*mAmGAUm | chr19: 55115649- |
| | | UAUCUGGUGAC | UAUCUGGUGAC | UmCCmUUmAUCUGmGU | 55115673 |
| | | AC | ACGUUGUAGCU | GAmCACmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025905 | 699 | AGGCAGAUUCC | AGGCAGAUUCC | mA*mG*mG*mCmAGAm | chr19: 55115650- |
| | | UUAUCUGGUGA | UUAUCUGGUGA | UmUCmCUmUAUCUmGG | 55115674 |
| | | CA | CAGUUGUAGCU | UGmACAmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025906 | 700 | UAGGCAGAUUC | UAGGCAGAUUC | mU*mA*mG*mGmCAGm | chr19: 55115651- |
| | | CUUAUCUGGUG | CUUAUCUGGUG | AmUUmCCmUUAUCmUG | 55115675 |
| | | AC | ACGUUGUAGCU | GUmGACmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025907 | 701 | UUAGGCAGAUU | UUAGGCAGAUU | mU*mU*mA*mGmGCAm | chr19: 55115652- |
| | | CCUUAUCUGGU | CCUUAUCUGGU | GmAUmUCmCUUAUmCU | 55115676 |
| | | GA | GAGUUGUAGCU | GGmUGAmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025908 | 702 | CUAACCCCCAC | CUAACCCCCAC | mC*mU*mA*mAmCCCm | chr19: 55115669- |
| | | CUCCUGUUAGG | CUCCUGUUAGG | CmCAmCCmUCCUGmUU | 55115693 |
| | | CA | CAGUUGUAGCU | AGmGCAmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025909 | 703 | CUCCUGAUAUU | CUCCUGAUAUU | mC*mU*mC*mCmUGAm | chr19: 55115684- |
| | | GGGUCUAACCC | GGGUCUAACCC | UmAUmUGmGGUCUmAA | 55115708 |
| | | CC | CCGUUGUAGCU | CCmCCCmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025910 | 704 | AGUCUCCUGAU | AGUCUCCUGAU | mA*mG*mU*mCmUCCm | chr19: 55115687- |
| | | AUUGGGUCUAA | AUUGGGUCUAA | UmGAmUAmUUGGGmUC | 55115711 |
| | | CC | CCGUUGUAGCU | UAmACCmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025911 | 705 | CCUAGUCUCCU | CCUAGUCUCCU | mC*mC*mU*mAmGUCm | chr19: 55115690- |
| | | GAUAUUGGGUC | GAUAUUGGGUC | UmCCmUGmAUAUUmGG | 55115714 |
| | | UA | UAGUUGUAGCU | GUmCUAmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025912 | 706 | UCCUAGUCUCC | UCCUAGUCUCC | mU*mC*mC*mUmAGUm | chr19: 55115691- |
| | | UGAUAUUGGGU | UGAUAUUGGGU | CmUCmCUmGAUAUmUG | 55115715 |
| | | CU | CUGUUGUAGCU | GGmUCUmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025913 | 707 | UUCCUAGUCUC | UUCCUAGUCUC | mU*mU*mC*mCmUAGm | chr19: 55115692- |
| | | CUGAUAUUGGG | CUGAUAUUGGG | UmCUmCCmUGAUAmUU | 55115716 |
| | | UC | UCGUUGUAGCU | GGmGUCmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025914 | 708 | CUUCCUAGUCU | CUUCCUAGUCU | mC*mU*mU*mCmCUAm | chr19: 55115693- |
| | | CCUGAUAUUGG | CCUGAUAUUGG | GmUCmUCmCUGAUmAU | 55115717 |
| | | GU | GUGUUGUAGCU | UGmGGUmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025915 | 709 | CAUCCUUAGGC | CAUCCUUAGGC | mC*mA*mU*mCmCUUm | chr19: 55115710- |
| | | CUCCUCCUUCC | CUCCUCCUUCC | AmGGmCCmUCCUCmCU | 55115734 |
| | | UA | UAGUUGUAGCU | UCmCUAmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025916 | 710 | AAAAGCCCCAU | AAAAGCCCCAU | mA*mA*mA*mAmGCCm | chr19: 55115718- |
| | | CCUUAGGCCUC | CCUUAGGCCUC | CmCAmUCmCUUAGmGC | 55115742 |
| | | CU | CUGUUGUAGCU | CUmCCUmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025917 | 711 | GUGACAGAAAA | GUGACAGAAAA | mG*mU*mG*mAmCAGm | chr19: 55115725- |
| | | GCCCCAUCCUU | GCCCCAUCCUU | AmAAmAGmCCCCAmUC | 55115749 |
| | | AG | AGGUUGUAGCU | CUmUAGmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025918 | 712 | UUGGUGACAGA | UUGGUGACAGA | mU*mU*mG*mGmUGAm | chr19: 55115728- |
| | | AAAGCCCCAUC | AAAGCCCCAUC | CmAGmAAmAAGCCmCC | 55115752 |
| | | CU | CUGUUGUAGCU | AUmCCUmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025919 | 713 | GACAGGAUUGG | GACAGGAUUGG | mG*mA*mC*mAmGGAm | chr19: 55115735- |
| | | UGACAGAAAAG | UGACAGAAAAG | UmUGmGUmGACAGmAA | 55115759 |
| | | CC | CCGUUGUAGCU | AAmGCCmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025920 | 714 | UAGGGACAGGA | UAGGGACAGGA | mU*mA*mG*mGmGACm | chr19: 55115739- |
| | | UUGGUGACAGA | UUGGUGACAGA | AmGGmAUmUGGUGmAC | 55115763 |
| | | AA | AAGUUGUAGCU | AGmAAAmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025921 | 715 | CUAGGGACAGG | CUAGGGACAGG | mC*mU*mA*mGmGGAm | chr19: 55115740- |
| | | AUUGGUGACAG | AUUGGUGACAG | CmAGmGAmUUGGUmGA | 55115764 |
| | | AA | AAGUUGUAGCU | CAmGAAmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025922 | 716 | ACUAGGGACAG | ACUAGGGACAG | mA*mC*mU*mAmGGGm | chr19: 55115741- |
| | | GAUUGGUGACA | GAUUGGUGACA | AmCAmGGmAUUGGmUG | 55115765 |
| | | GA | GAGUUGUAGCU | ACmAGAmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025923 | 717 | CUGGGUACUUU | CUGGGUACUUU | mC*mU*mG*mGmGUAm | chr19: 55115782- |
| | | UAUCUGUCCCC | UAUCUGUCCCC | CmUUmUUmAUCUGmUC | 55115806 |
| | | UC | UCGUUGUAGCU | CCmCUCmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025924 | 718 | UCUGGGUACUU | UCUGGGUACUU | mU*mC*mU*mGmGGUm | chr19: 55115783- |
| | | UUAUCUGUCCC | UUAUCUGUCCC | AmCUmUUmUAUCUmGU | 55115807 |
| | | CU | CUGUUGUAGCU | CCmCCUmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025925 | 719 | UUCUGGGUACU | UUCUGGGUACU | mU*mU*mC*mUmGGGm | chr19: 55115784- |
| | | UUUAUCUGUCC | UUUAUCUGUCC | UmACmUUmUUAUCmUG | 55115808 |
| | | CC | CCGUUGUAGCU | UCmCCCmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025926 | 720 | UGGUUCUGGGU | UGGUUCUGGGU | mU*mG*mG*mUmUCUm | chr19: 55115787- |
| | | ACUUUUAUCUG | ACUUUUAUCUG | GmGGmUAmCUUUUmAU | 55115811 |
| | | UC | UCGUUGUAGCU | CUmGUCmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025927 | 721 | CUCUGGUUCUG | CUCUGGUUCUG | mC*mU*mC*mUmGGUm | chr19: 55115790- |
| | | GGUACUUUUAU | GGUACUUUUAU | UmCUmGGmGUACUmUU | 55115814 |
| | | CU | CUGUUGUAGCU | UAmUCUmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025928 | 722 | GCUCUGGUUCU | GCUCUGGUUCU | mG*mC*mU*mCmUGGm | chr19: 55115791- |
| | | GGGUACUUUUA | GGGUACUUUUA | UmUCmUGmGGUACmUU | 55115815 |
| | | UC | UCGUUGUAGCU | UUmAUCmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025929 | 723 | GGCUCUGGUUC | GGCUCUGGUUC | mG*mG*mC*mUmCUGm | chr19: 55115792- |
| | | UGGGUACUUUU | UGGGUACUUUU | GmUUmCUmGGGUAmCU | 55115816 |
| | | AU | AUGUUGUAGCU | UUmUAUmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025930 | 724 | GAGCUGGGACC | GAGCUGGGACC | mG*mA*mG*mCmUGGm | chr19: 55115831- |
| | | ACCUUAUAUUC | ACCUUAUAUUC | GmACmCAmCCUUAmUA | 55115855 |
| | | CC | CCGUUGUAGCU | UUmCCCmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025931 | 725 | GUCCCCGAGCU | GUCCCCGAGCU | mG*mU*mC*mCmCCGm | chr19: 55115837- |
| | | GGGACCACCUU | GGGACCACCUU | AmGCmUGmGGACCmAC | 55115861 |
| | | AU | AUGUUGUAGCU | CUmUAUmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025932 | 726 | UGUCCCCGAGC | UGUCCCCGAGC | mU*mG*mU*mCmCCCm | chr19: 55115838- |
| | | UGGGACCACCU | UGGGACCACCU | GmAGmCUmGGGACmCA | 55115862 |
| | | UA | UAGUUGUAGCU | CCmUUAmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025933 | 727 | CCAGGGAUCCU | CCAGGGAUCCU | mC*mC*mA*mGmGGAm | chr19: 55115850- |
| | | GUGUCCCCGAG | GUGUCCCCGAG | UmCCmUGmUGUCCmCC | 55115874 |
| | | CU | CUGUUGUAGCU | GAmGCUmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025934 | 728 | GUUUGCUGCCU | GUUUGCUGCCU | mG*mU*mU*mUmGCUm | chr19: 55115861- |
| | | CCAGGGAUCCU | CCAGGGAUCCU | GmCCmUCmCAGGGmAU | 55115885 |
| | | GU | GUGUUGUAGCU | CCmUGUmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025935 | 729 | UGUUUGCUGCC | UGUUUGCUGCC | mU*mG*mU*mUmUGCm | chr19: 55115862- |
| | | UCCAGGGAUCC | UCCAGGGAUCC | UmGCmCUmCCAGGmGA | 55115886 |
| | | UG | UGGUUGUAGCU | UCmCUGmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025936 | 730 | AUGUUUGCUGC | AUGUUUGCUGC | mA*mU*mG*mUmUUGm | chr19: 55115863- |
| | | CUCCAGGGAUC | CUCCAGGGAUC | CmUGmCCmUCCAGmGG | 55115887 |
| | | CU | CUGUUGUAGCU | AUmCCUmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025937 | 731 | GGACAGCAUGU | GGACAGCAUGU | mG*mG*mA*mCmAGCm | chr19: 55115870- |
| | | UUGCUGCCUCC | UUGCUGCCUCC | AmUGmUUmUGCUGmCC | 55115894 |
| | | AG | AGGUUGUAGCU | UCmCAGmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025938 | 732 | CCACUUCAGGA | CCACUUCAGGA | mC*mC*mA*mCmUUCm | chr19: 55115878- |
| | | CAGCAUGUUUG | CAGCAUGUUUG | AmGGmACmAGCAUmGU | 55115902 |
| | | CU | CUGUUGUAGCU | UUmGCUmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025939 | 733 | UGUCCACUUCA | UGUCCACUUCA | mU*mG*mU*mCmCACm | chr19: 55115881- |
| | | GGACAGCAUGU | GGACAGCAUGU | UmUCmAGmGACAGmCA | 55115905 |
| | | UU | UUGUUGUAGCU | UGmUUUmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025940 | 734 | CUUCUUCCUCC | CUUCUUCCUCC | mC*mU*mU*mCmUUCm | chr19: 55115906- |
| | | AACCCGGGCCC | AACCCGGGCCC | CmUCmCAmACCCGmGG | 55115930 |
| | | CU | CUGUUGUAGCU | CCmCCUmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025941 | 735 | UCAGCUAGUCU | UCAGCUAGUCU | mU*mC*mA*mGmCUAm | chr19: 55115915- |
| | | UCUUCCUCCAA | UCUUCCUCCAA | GmUCmUUmCUUCCmUC | 55115939 |
| | | CC | CCGUUGUAGCU | CAmACCmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025942 | 736 | AGAGCUCAGCU | AGAGCUCAGCU | mA*mG*mA*mGmCUCm | chr19: 55115920- |
| | | AGUCUUCUUCC | AGUCUUCUUCC | AmGCmUAmGUCUUmCU | 55115944 |
| | | UC | UCGUUGUAGCU | UCmCUCmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025943 | 737 | GAGAGCUCAGC | GAGAGCUCAGC | mG*mA*mG*mAmGCUm | chr19: 55115921- |
| | | UAGUCUUCUUC | UAGUCUUCUUC | CmAGmCUmAGUCUmUC | 55115945 |
| | | CU | CUGUUGUAGCU | UUmCCUmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025944 | 738 | GUCCGAGAGCU | GUCCGAGAGCU | mG*mU*mC*mCmGAGm | chr19: 55115925- |
| | | CAGCUAGUCUU | CAGCUAGUCUU | AmGCmUCmAGCUAmGU | 55115949 |
| | | CU | CUGUUGUAGCU | CUmUCUmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025945 | 739 | GCCCCCUGUCA | GCCCCCUGUCA | mG*mC*mC*mCmCCUm | chr19: 55115951- |
| | | UGGCAUCUUCC | UGGCAUCUUCC | GmUCmAUmGGCAUmCU | 55115975 |
| | | AG | AGGUUGUAGCU | UCmCAGmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025946 | 740 | CUCUUCCAGCC | CUCUUCCAGCC | mC*mU*mC*mUmUCCm | chr19: 55115959- |
| | | CCCUGUCAUGG | CCCUGUCAUGG | AmGCmCCmCCUGUmCA | 55115983 |
| | | CA | CAGUUGUAGCU | UGmGCAmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025947 | 741 | CUCUAGUCUGU | CUCUAGUCUGU | mC*mU*mC*mUmAGUm | chr19: 55115975- |
| | | GCUAGCUCUUC | GCUAGCUCUUC | CmUGmUGmCUAGCmUC | 55115999 |
| | | CA | CAGUUGUAGCU | UUmCCAmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025948 | 742 | UCUCUAGUCUG | UCUCUAGUCUG | mU*mC*mU*mCmUAGm | chr19: 55115976- |
| | | UGCUAGCUCUU | UGCUAGCUCUU | UmCUmGUmGCUAGmCU | 55116000 |
| | | CC | CCGUUGUAGCU | CUmUCCmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025949 | 743 | CUCUCUAGUCU | CUCUCUAGUCU | mC*mU*mC*mUmCUAm | chr19: 55115977- |
| | | GUGCUAGCUCU | GUGCUAGCUCU | GmUCmUGmUGCUAmGC | 55116001 |
| | | UC | UCGUUGUAGCU | UCmUUCmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025950 | 744 | CCUCUCUAGUC | CCUCUCUAGUC | mC*mC*mU*mCmUCUm | chr19: 55115978- |
| | | UGUGCUAGCUC | UGUGCUAGCUC | AmGUmCUmGUGCUmAG | 55116002 |
| | | UU | UUGUUGUAGCU | CUmCUUmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025951 | 745 | CUUACCUCUCU | CUUACCUCUCU | mC*mU*mU*mAmCCUm | chr19: 55115982- |
| | | AGUCUGUGCUA | AGUCUGUGCUA | CmUCmUAmGUCUGmUG | 55116006 |
| | | GC | GCGUUGUAGCU | CUmAGCmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025952 | 746 | CAUUUGGGCAG | CAUUUGGGCAG | mC*mA*mU*mUmUGGm | chr19: 55116006- |
| | | CUCCCCUACCC | CUCCCCUACCC | GmCAmGCmUCCCCmUA | 55116030 |
| | | CC | CCGUUGUAGCU | CCmCCCmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025953 | 747 | CUCCUUUCAUU | CUCCUUUCAUU | mC*mU*mC*mCmUUUm | chr19: 55116013- |
| | | UGGGCAGCUCC | UGGGCAGCUCC | CmAUmUUmGGGCAmGC | 55116037 |
| | | CC | CCGUUGUAGCU | UCmCCCmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025954 | 748 | ACUCCUUUCAU | ACUCCUUUCAU | mA*mC*mU*mCmCUUm | chr19: 55116014- |
| | | UUGGGCAGCUC | UUGGGCAGCUC | UmCAmUUmUGGGCmAG | 55116038 |
| | | CC | CCGUUGUAGCU | CUmCCCmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025955 | 749 | CACUCCUUUCA | CACUCCUUUCA | mC*mA*mC*mUmCCUm | chr19: 55116015- |
| | | UUUGGGCAGCU | UUUGGGCAGCU | UmUCmAUmUUGGGmCA | 55116039 |
| | | CC | CCGUUGUAGCU | GCmUCCmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025956 | 750 | CUCUCACUCCU | CUCUCACUCCU | mC*mU*mC*mUmCACm | chr19: 55116019- |
| | | UUCAUUUGGGC | UUCAUUUGGGC | UmCCmUUmUCAUUmUG | 55116043 |
| | | AG | AGGUUGUAGCU | GGmCAGmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025957 | 751 | CCUCUCACUCC | CCUCUCACUCC | mC*mC*mU*mCmUCAm | chr19: 55116020- |
| | | UUUCAUUUGGG | UUUCAUUUGGG | CmUCmCUmUUCAUmUU | 55116044 |
| | | CA | CAGUUGUAGCU | GGmGCAmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025958 | 752 | ACCUCUCACUC | ACCUCUCACUC | mA*mC*mC*mUmCUCm | chr19: 55116021- |
| | | CUUUCAUUUGG | CUUUCAUUUGG | AmCUmCCmUUUCAmUU | 55116045 |
| | | GC | GCGUUGUAGCU | UGmGGCmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025959 | 753 | CUCCUGUGGAU | CUCCUGUGGAU | mC*mU*mC*mCmUGUm | chr19: 55116039- |
| | | UCGGGUCACCU | UCGGGUCACCU | GmGAmUUmCGGGUmCA | 55116063 |
| | | CU | CUGUUGUAGCU | CCmUCUmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025960 | 754 | CACCCCGUUCU | CACCCCGUUCU | mC*mA*mC*mCmCCGm | chr19: 55116048- |
| | | CCUGUGGAUUC | CCUGUGGAUUC | UmUCmUCmCUGUGmGA | 55116072 |
| | | GG | GGGUUGUAGCU | UUmCGGmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025961 | 755 | UGCUUUCUUUG | UGCUUUCUUUG | mU*mG*mC*mUmUUCm | chr19: 55116065- |
| | | CCUGGACACCC | CCUGGACACCC | UmUUmGCmCUGGAmCA | 55116089 |
| | | CG | CGGUUGUAGCU | CCmCCGmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025962 | 756 | AUCCUCUUGCU | AUCCUCUUGCU | mA*mU*mC*mCmUCUm | chr19: 55116072- |
| | | UUCUUUGCCUG | UUCUUUGCCUG | UmGCmUUmUCUUUmGC | 55116096 |
| | | GA | GAGUUGUAGCU | CUmGGAmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025963 | 757 | CAUCCUCUUGC | CAUCCUCUUGC | mC*mA*mU*mCmCUCm | chr19: 55116073- |
| | | UUUCUUUGCCU | UUUCUUUGCCU | UmUGmCUmUUCUUmUG | 55116097 |
| | | GG | GGGUUGUAGCU | CCmUGGmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025964 | 758 | CCAUCCUCUUG | CCAUCCUCUUG | mC*mC*mA*mUmCCUm | chr19: 55116074- |
| | | CUUUCUUUGCC | CUUUCUUUGCC | CmUUmGCmUUUCUmUU | 55116098 |
| | | UG | UGGUUGUAGCU | GCmCUGmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025965 | 759 | CCACCUCUCCA | CCACCUCUCCA | mC*mC*mA*mCmCUCm | chr19: 55116082- |
| | | UCCUCUUGCUU | UCCUCUUGCUU | UmCCmAUmCCUCUmUG | 55116106 |
| | | UC | UCGUUGUAGCU | CUmUUCmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025966 | 760 | GUCUCCCUGGC | GUCUCCCUGGC | mG*mU*mC*mUmCCCm | chr19: 55116098- |
| | | UUUAGCCACCU | UUUAGCCACCU | UmGGmCUmUUAGCmCA | 55116122 |
| | | CU | CUGUUGUAGCU | CCmUCUmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025967 | 76 | CCCCGUCUCCC | CCCCGUCUCCC | mC*mC*mC*mCmGUCm | chr19: 55116102- |
| | | UGGCUUUAGCC | UGGCUUUAGCC | UmCCmCUmGGCUUmUA | 55116126 |
| | | AC | ACGUUGUAGCU | GCmCACmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025968 | 762 | AAGUACCCCGU | AAGUACCCCGU | mA*mA*mG*mUmACCm | chr19: 55116107- |
| | | CUCCCUGGCUU | CUCCCUGGCUU | CmCGmUCmUCCCUmGG | 55116131 |
| | | UA | UAGUUGUAGCU | CUmUUAmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025969 | 763 | CCAAAGUACCC | CCAAAGUACCC | mC*mC*mA*mAmAGUm | chr19: 55116110- |
| | | CGUCUCCCUGG | CGUCUCCCUGG | AmCCmCCmGUCUCmCC | 55116134 |
| | | CU | CUGUUGUAGCU | UGmGCUmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025970 | 764 | UCUGGACAACC | UCUGGACAACC | mU*mC*mU*mGmGACm | chr19: 55116121- |
| | | CCAAAGUACCC | CCAAAGUACCC | AmACmCCmCAAAGmUA | 55116145 |
| | | CG | CGGUUGUAGCU | CCmCCGmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025971 | 765 | UUCUGGACAAC | UUCUGGACAAC | mU*mU*mC*mUmGGAm | chr19: 55116122- |
| | | CCCAAAGUACC | CCCAAAGUACC | CmAAmCCmCCAAAmGU | 55116146 |
| | | CC | CCGUUGUAGCU | ACmCCCmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025972 | 766 | CCGUUUUUCUG | CCGUUUUUCUG | mC*mC*mG*mUmUUUm | chr19: 55116128- |
| | | GACAACCCCAA | GACAACCCCAA | UmCUmGGmACAACmCC | 55116152 |
| | | AG | AGGUUGUAGCU | CAmAAGmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025973 | 767 | ACCGUUUUUCU | ACCGUUUUUCU | mA*mC*mC*mGmUUUm | chr19: 55116129- |
| | | GGACAACCCCA | GGACAACCCCA | UmUCmUGmGACAAmCC | 55116153 |
| | | AA | AAGUUGUAGCU | CCmAAAmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025974 | 768 | CACCGUUUUUC | CACCGUUUUUC | mC*mA*mC*mCmGUUm | chr19: 55116130- |
| | | UGGACAACCCC | UGGACAACCCC | UmUUmCUmGGACAmAC | 55116154 |
| | | AA | AAGUUGUAGCU | CCmCAAmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025975 | 769 | UGCAUCAUCAC | UGCAUCAUCAC | mU*mG*mC*mAmUCAm | chr19: 55116138- |
| | | CGUUUUUCUGG | CGUUUUUCUGG | UmCAmCCmGUUUUmUC | 55116162 |
| | | AC | ACGUUGUAGCU | UGmGACmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025976 | 770 | CUGCAUCAUCA | CUGCAUCAUCA | mC*mU*mG*mCmAUCm | chr19: 55116139- |
| | | CCGUUUUUCUG | CCGUUUUUCUG | AmUCmACmCGUUUmUU | 55116163 |
| | | GA | GAGUUGUAGCU | CUmGGAmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025977 | 771 | CCUGCAUCAUC | CCUGCAUCAUC | mC*mC*mU*mGmCAUm | chr19: 55116140- |
| | | ACCGUUUUUCU | ACCGUUUUUCU | CmAUmCAmCCGUUmUU | 55116164 |
| | | GG | GGGUUGUAGCU | UCmUGGmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025978 | 772 | CUCCCCUUCUU | CUCCCCUUCUU | mC*mU*mC*mCmCCUm | chr19: 55116156- |
| | | GUAGGCCUGCA | GUAGGCCUGCA | UmCUmUGmUAGGCmCU | 55116180 |
| | | UC | UCGUUGUAGCU | GCmAUCmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025979 | 773 | CUUGCGUCCCG | CUUGCGUCCCG | mC*mU*mU*mGmCGUm | chr19: 55116168- |
| | | CCUCCCCUUCU | CCUCCCCUUCU | CmCCmGCmCUCCCmCU | 55116192 |
| | | UG | UGGUUGUAGCU | UCmUUGmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |
| G025980 | 774 | CUCCGACGGAU | CUCCGACGGAU | mC*mU*mC*mCmGACm | chr19: 55116185- |
| | | GUCUCCCUUGC | GUCUCCCUUGC | GmGAmUGmUCUCCmCU | 55116209 |
| | | GU | GUGUUGUAGCU | UGmCGUmGUUGmUmAm | |
| | | | CCCUGAAACCG | GmCUCCCmUmGmAmAm | |
| | | | UUGCUACAAUA | AmCmCGUUmGmCUAmC | |
| | | | AGGCCGUCGAA | AAU*AAGmGmCCmGmU | |
| | | | AGAUGUGCCGC | mCmGmAmAmAmGmAmU | |
| | | | AACGCUCUGCC | GUGCmCGmCAAmCGCU | |
| | | | UUCUGGCAUCG | CUmGmCCmUmUmCmUG | |
| | | | UU | GCAUCG*mU*mU | |

The terms “mA,” “mc,” “mU,” or “mG” may be used to denote a nucleotide that has been modified with 2′—O-Me.

In some embodiments, the AAVS1 guide RNA comprises a guide sequence selected from SEQ TD NOs: 601-774. In some embodiments, the AAVS1 guide RNA comprises a guide sequence that is at least 19, 20, 21, 22, 23, or 24 contiguous nucleotides of a sequence selected from SEQ ID NOs: 601-774. In some embodiments, the AAVS1 guide RNA comprises a guide sequence that is at least 95%, 90%, or 85% identical to a sequence selected from SEQ ID NOs: 601-774. In some embodiments, the AAVS1 guide RNA comprises a guide sequence that is at least 95% identical to a sequence selected from SEQ ID NOs: 601-774.

In some embodiments, the AAVS1 guide RNA comprises a guide sequence that comprises at least 10 contiguous nucleotides±10 nucleotides of a genomic coordinate listed in Table 5. As used herein, at least 10 contiguous nucleotides±10 nucleotides of a genomic coordinate means, for example, at least 10 contiguous nucleotides within the genomic coordinates wherein the genomic coordinates include 10 nucleotides in the 5′ direction and 10 nucleotides in the 3′ direction from the ranges listed in Table 5. For example, an AAVS1 guide RNA may comprise 10 contiguous nucleotides within the genomic coordinates chosen from: chr19:55115218-55115242; chr19:55115477-55115501; chr19:55115504-55115528; chr19:55115513-55115537; chr19:55115514-55115538; chr19:55115517-55115541; chr19:55115518-55115542; chr19:55115549-55115573; chr19:55115574-55115598; chr19:55115606-55115630; chr19:55115933-55115957; chr19:55116026-55116050; chr19:55116045-55116069; chr19:55116084-55116108; chr19:55115276-55115300; chr19:55115509-55115533; chr19:55115579-55115603; chr19:55115863-55115887; chr19:55115906-55115930; or chr19:55116006-55116030; including the boundary nucleotides of these ranges. In some embodiments, the AAVS1 guide RNA comprises a guide sequence that is at least 19, 20, 21, 22, 23, or 24 contiguous nucleotides of a sequence that comprises 10 contiguous nucleotides±10 nucleotides of a genomic coordinate listed in Table 5. In some embodiments, the AAVS1 guide RNA comprises a guide sequence that is at least 95%, 90%, or 85% identical to a sequence selected from a sequence that is 19, 20, 21, 22, 23, or 24 contiguous nucleotides of a sequence that comprises 10 contiguous nucleotides±10 nucleotides of a genomic coordinate listed in Table 5.

In some embodiments, the AAVS1 guide RNA comprises a guide sequence that comprises at least 15 contiguous nucleotides±10 nucleotides of a genomic coordinate listed in Table 5. In some embodiments, the AAVS1 guide RNA comprises a guide sequence that comprises at least 24 contiguous nucleotides±10 nucleotides of a genomic coordinate listed in Table 5.

In some embodiments, the AAVS1 guide RNA comprises SEQ ID NO: 601. In some embodiments, the AAVS1 guide RNA comprises SEQ ID NO: 602. In some embodiments, the AAVS1 guide RNA comprises SEQ ID NO: 603. In some embodiments, the AAVS1 guide RNA comprises SEQ ID NO: 604. In some embodiments, the AAVS1 guide RNA comprises SEQ ID NO: 605. In some embodiments, the AAVS1 guide RNA comprises SEQ ID NO: 606. In some embodiments, the AAVS1 guide RNA comprises SEQ ID NO: 607. In some embodiments, the AAVS1 guide RNA comprises SEQ ID NO: 608. In some embodiments, the AAVS1 guide RNA comprises SEQ ID NO: 609. In some embodiments, the AAVS1 guide RNA comprises SEQ ID NO: 610. In some embodiments, the AAVS1 guide RNA comprises SEQ ID NO: 611. In some embodiments, the AAVS1 guide RNA comprises SEQ ID NO: 612. In some embodiments, the AAVS1 guide RNA comprises SEQ ID NO: 613. In some embodiments, the AAVS1 guide RNA comprises SEQ ID NO: 614. In some embodiments, the AAVS1 guide RNA comprises SEQ ID NO: 615. In some embodiments, the AAVS1 guide RNA comprises SEQ ID NO: 616. In some embodiments, the AAVS1 guide RNA comprises SEQ ID NO: 617. In some embodiments, the AAVS1 guide RNA comprises SEQ ID NO: 618. In some embodiments, the AAVS1 guide RNA comprises SEQ ID NO: 619. In some embodiments, the AAVS1 guide RNA comprises SEQ ID NO: 620. In some embodiments, the AAVS1 guide RNA comprises SEQ ID NO: 621. In some embodiments, the AAVS1 guide RNA comprises SEQ ID NO: 622. In some embodiments, the AAVS1 guide RNA comprises SEQ ID NO: 623. In some embodiments, the AAVS1 guide RNA comprises SEQ ID NO: 624. In some embodiments, the AAVS1 guide RNA comprises SEQ ID NO: 625. In some embodiments, the AAVS1 guide RNA comprises SEQ ID NO: 626. In some embodiments, the AAVS1 guide RNA comprises SEQ ID NO: 627. In some embodiments, the AAVS1 guide RNA comprises SEQ ID NO: 628. In some embodiments, the AAVS1 guide RNA comprises SEQ ID NO: 629. In some embodiments, the AAVS1 guide RNA comprises SEQ ID NO: 630. In some embodiments, the AAVS1 guide RNA comprises SEQ ID NO: 631. In some embodiments, the AAVS1 guide RNA comprises SEQ ID NO: 632. In some embodiments, the AAVS1 guide RNA comprises SEQ ID NO: 633. In some embodiments, the AAVS1 guide RNA comprises SEQ ID NO: 634. In some embodiments, the AAVS1 guide RNA comprises SEQ ID NO: 635. In some embodiments, the AAVS1 guide RNA comprises SEQ ID NO: 636. In some embodiments, the AAVS1 guide RNA comprises SEQ ID NO: 637. In some embodiments, the AAVS1 guide RNA comprises SEQ ID NO: 638. In some embodiments, the AAVS1 guide RNA comprises SEQ ID NO: 639. In some embodiments, the AAVS1 guide RNA comprises SEQ ID NO: 640. In some embodiments, the AAVS1 guide RNA comprises SEQ ID NO: 641. In some embodiments, the AAVS1 guide RNA comprises SEQ ID NO: 642. In some embodiments, the AAVS1 guide RNA comprises SEQ ID NO: 643. In some embodiments, the AAVS1 guide RNA comprises SEQ ID NO: 644. In some embodiments, the AAVS1 guide RNA comprises SEQ ID NO: 645. In some embodiments, the AAVS1 guide RNA comprises SEQ ID NO: 646. In some embodiments, the AAVS1 guide RNA comprises SEQ ID NO: 647. In some embodiments, the AAVS1 guide RNA comprises SEQ ID NO: 648. In some embodiments, the AAVS1 guide RNA comprises SEQ ID NO: 649. In some embodiments, the AAVS1 guide RNA comprises SEQ ID NO: 650. In some embodiments, the AAVS1 guide RNA comprises SEQ ID NO: 651. In some embodiments, the AAVS1 guide RNA comprises SEQ ID NO: 652. In some embodiments, the AAVS1 guide RNA comprises SEQ ID NO: 653. In some embodiments, the AAVS1 guide RNA comprises SEQ ID NO: 654. In some embodiments, the AAVS1 guide RNA comprises SEQ ID NO: 655. In some embodiments, the AAVS1 guide RNA comprises SEQ ID NO: 656. In some embodiments, the AAVS1 guide RNA comprises SEQ ID NO: 657. In some embodiments, the AAVS1 guide RNA comprises SEQ ID NO: 658. In some embodiments, the AAVS1 guide RNA comprises SEQ ID NO: 659. In some embodiments, the AAVS1 guide RNA comprises SEQ ID NO: 660. In some embodiments, the AAVS1 guide RNA comprises SEQ ID NO: 661. In some embodiments, the AAVS1 guide RNA comprises SEQ ID NO: 662. In some embodiments, the AAVS1 guide RNA comprises SEQ ID NO: 663. In some embodiments, the AAVS1 guide RNA comprises SEQ ID NO: 664. In some embodiments, the AAVS1 guide RNA comprises SEQ ID NO: 665. In some embodiments, the AAVS1 guide RNA comprises SEQ ID NO: 666. In some embodiments, the AAVS1 guide RNA comprises SEQ ID NO: 667. In some embodiments, the AAVS1 guide RNA comprises SEQ ID NO: 668. In some embodiments, the AAVS1 guide RNA comprises SEQ ID NO: 669. In some embodiments, the AAVS1 guide RNA comprises SEQ ID NO: 670. In some embodiments, the AAVS1 guide RNA comprises SEQ ID NO: 671. In some embodiments, the AAVS1 guide RNA comprises SEQ ID NO: 672. In some embodiments, the AAVS1 guide RNA comprises SEQ ID NO: 673. In some embodiments, the AAVS1 guide RNA comprises SEQ ID NO: 674. In some embodiments, the AAVS1 guide RNA comprises SEQ ID NO: 675. In some embodiments, the AAVS1 guide RNA comprises SEQ ID NO: 676. In some embodiments, the AAVS1 guide RNA comprises SEQ ID NO: 677. In some embodiments, the AAVS1 guide RNA comprises SEQ ID NO: 678. In some embodiments, the AAVS1 guide RNA comprises SEQ ID NO: 679. In some embodiments, the AAVS1 guide RNA comprises SEQ ID NO: 680. In some embodiments, the AAVS1 guide RNA comprises SEQ ID NO: 681. In some embodiments, the AAVS1 guide RNA comprises SEQ ID NO: 682. In some embodiments, the AAVS1 guide RNA comprises SEQ ID NO: 683. In some embodiments, the AAVS1 guide RNA comprises SEQ ID NO: 684. In some embodiments, the AAVS1 guide RNA comprises SEQ ID NO: 685. In some embodiments, the AAVS1 guide RNA comprises SEQ ID NO: 686. In some embodiments, the AAVS1 guide RNA comprises SEQ ID NO: 687. In some embodiments, the AAVS1 guide RNA comprises SEQ ID NO: 688. In some embodiments, the AAVS1 guide RNA comprises SEQ ID NO: 689. In some embodiments, the AAVS1 guide RNA comprises SEQ ID NO: 690. In some embodiments, the AAVS1 guide RNA comprises SEQ ID NO: 691. In some embodiments, the AAVS1 guide RNA comprises SEQ ID NO: 692. In some embodiments, the AAVS1 guide RNA comprises SEQ ID NO: 693. In some embodiments, the AAVS1 guide RNA comprises SEQ ID NO: 694. In some embodiments, the AAVS1 guide RNA comprises SEQ ID NO: 695. In some embodiments, the AAVS1 guide RNA comprises SEQ ID NO: 696. In some embodiments, the AAVS1 guide RNA comprises SEQ ID NO: 697. In some embodiments, the AAVS1 guide RNA comprises SEQ ID NO: 698. In some embodiments, the AAVS1 guide RNA comprises SEQ ID NO: 699. In some embodiments, the AAVS1 guide RNA comprises SEQ ID NO: 700. In some embodiments, the AAVS1 guide RNA comprises SEQ ID NO: 701. In some embodiments, the AAVS1 guide RNA comprises SEQ ID NO: 702. In some embodiments, the AAVS1 guide RNA comprises SEQ ID NO: 703. In some embodiments, the AAVS1 guide RNA comprises SEQ ID NO: 704. In some embodiments, the AAVS1 guide RNA comprises SEQ ID NO: 705. In some embodiments, the AAVS1 guide RNA comprises SEQ ID NO: 706. In some embodiments, the AAVS1 guide RNA comprises SEQ ID NO: 707. In some embodiments, the AAVS1 guide RNA comprises SEQ ID NO: 708. In some embodiments, the AAVS1 guide RNA comprises SEQ ID NO: 709. In some embodiments, the AAVS1 guide RNA comprises SEQ ID NO: 710. In some embodiments, the AAVS1 guide RNA comprises SEQ ID NO: 711. In some embodiments, the AAVS1 guide RNA comprises SEQ ID NO: 712. In some embodiments, the AAVS1 guide RNA comprises SEQ ID NO: 713. In some embodiments, the AAVS1 guide RNA comprises SEQ ID NO: 714. In some embodiments, the AAVS1 guide RNA comprises SEQ ID NO: 715. In some embodiments, the AAVS1 guide RNA comprises SEQ ID NO: 716. In some embodiments, the AAVS1 guide RNA comprises SEQ ID NO: 717. In some embodiments, the AAVS1 guide RNA comprises SEQ ID NO: 718. In some embodiments, the AAVS1 guide RNA comprises SEQ ID NO: 719. In some embodiments, the AAVS1 guide RNA comprises SEQ ID NO: 720. In some embodiments, the AAVS1 guide RNA comprises SEQ ID NO: 721. In some embodiments, the AAVS1 guide RNA comprises SEQ ID NO: 722. In some embodiments, the AAVS1 guide RNA comprises SEQ ID NO: 723. In some embodiments, the AAVS1 guide RNA comprises SEQ ID NO: 724. In some embodiments, the AAVS1 guide RNA comprises SEQ ID NO: 725. In some embodiments, the AAVS1 guide RNA comprises SEQ ID NO: 726. In some embodiments, the AAVS1 guide RNA comprises SEQ ID NO: 727. In some embodiments, the AAVS1 guide RNA comprises SEQ ID NO: 728. In some embodiments, the AAVS1 guide RNA comprises SEQ ID NO: 729. In some embodiments, the AAVS1 guide RNA comprises SEQ ID NO: 730. In some embodiments, the AAVS1 guide RNA comprises SEQ ID NO: 731. In some embodiments, the AAVS1 guide RNA comprises SEQ ID NO: 732. In some embodiments, the AAVS1 guide RNA comprises SEQ ID NO: 733. In some embodiments, the AAVS1 guide RNA comprises SEQ ID NO: 734. In some embodiments, the AAVS1 guide RNA comprises SEQ ID NO: 735. In some embodiments, the AAVS1 guide RNA comprises SEQ ID NO: 736. In some embodiments, the AAVS1 guide RNA comprises SEQ ID NO: 737. In some embodiments, the AAVS1 guide RNA comprises SEQ ID NO: 738. In some embodiments, the AAVS1 guide RNA comprises SEQ ID NO: 739. In some embodiments, the AAVS1 guide RNA comprises SEQ ID NO: 740. In some embodiments, the AAVS1 guide RNA comprises SEQ ID NO: 741. In some embodiments, the AAVS1 guide RNA comprises SEQ ID NO: 742. In some embodiments, the AAVS1 guide RNA comprises SEQ ID NO: 743. In some embodiments, the AAVS1 guide RNA comprises SEQ ID NO: 744. In some embodiments, the AAVS1 guide RNA comprises SEQ ID NO: 745. In some embodiments, the AAVS1 guide RNA comprises SEQ ID NO: 746. In some embodiments, the AAVS1 guide RNA comprises SEQ ID NO: 747. In some embodiments, the AAVS1 guide RNA comprises SEQ ID NO: 748. In some embodiments, the AAVS1 guide RNA comprises SEQ ID NO: 749. In some embodiments, the AAVS1 guide RNA comprises SEQ ID NO: 750. In some embodiments, the AAVS1 guide RNA comprises SEQ ID NO: 751. In some embodiments, the AAVS1 guide RNA comprises SEQ ID NO: 752. In some embodiments, the AAVS1 guide RNA comprises SEQ ID NO: 753. In some embodiments, the AAVS1 guide RNA comprises SEQ ID NO: 754. In some embodiments, the AAVS1 guide RNA comprises SEQ ID NO: 755. In some embodiments, the AAVS1 guide RNA comprises SEQ ID NO: 756. In some embodiments, the AAVS1 guide RNA comprises SEQ ID NO: 757. In some embodiments, the AAVS1 guide RNA comprises SEQ ID NO: 758. In some embodiments, the AAVS1 guide RNA comprises SEQ ID NO: 759. In some embodiments, the AAVS1 guide RNA comprises SEQ ID NO: 760. In some embodiments, the AAVS1 guide RNA comprises SEQ ID NO: 761. In some embodiments, the AAVS1 guide RNA comprises SEQ ID NO: 762. In some embodiments, the AAVS1 guide RNA comprises SEQ ID NO: 763. In some embodiments, the AAVS1 guide RNA comprises SEQ ID NO: 764. In some embodiments, the AAVS1 guide RNA comprises SEQ ID NO: 765. In some embodiments, the AAVS1 guide RNA comprises SEQ ID NO: 766. In some embodiments, the AAVS1 guide RNA comprises SEQ ID NO: 767. In some embodiments, the AAVS1 guide RNA comprises SEQ ID NO: 768. In some embodiments, the AAVS1 guide RNA comprises SEQ ID NO: 769. In some embodiments, the AAVS1 guide RNA comprises SEQ ID NO: 770. In some embodiments, the AAVS1 guide RNA comprises SEQ ID NO: 771. In some embodiments, the AAVS1 guide RNA comprises SEQ ID NO: 772. In some embodiments, the AAVS1 guide RNA comprises SEQ ID NO: 773. In some embodiments, the AAVS1 guide RNA comprises SEQ ID NO: 774.

In some embodiments, the AAVS1 guide RNA comprises a guide sequence of any one of: SEQ ID NOs: 611, 620, 622, 626-629, 632-634, 656, 659-661, 673, 691-692, 730, 734, and 746.

In some embodiments, the AAVS1 guide RNA comprises a guide sequence of any one of: SEQ ID NOs: 611, 620, 622, 627-629, 636, 656, 659-661, and 673,

In some embodiments, the AAVS1 guide RNA comprises a guide sequence of any one of: SEQ ID NOs: 692, 709, 730, 734, 746, 748, 760-761, and 763.

Additional embodiments of AAVS1 guide RNAs are provided herein, including e.g., exemplary modifications to the guide RNA.

### 1. Genetic Modifications to AAVS1

In some embodiments, the methods and compositions disclosed herein genetically modify at least one nucleotide of the AAVS1 locus in a cell. Genetic modifications encompass the population of modifications that results from contact with a genomic editing system (e.g., the population of edits that result from Cas9 and an AAVS1 guide RNA, or the population of edits that result from BC22 and an AAVS1 guide RNA).

In some embodiments, the genetic modification comprises at least one nucleotide within the genomic coordinates chr19:55115151-55116209. In some embodiments, the genetic modification comprises at least one nucleotide within the genomic coordinates chosen from any of the genomic coordinates listed in Table 5.

In some embodiments, the genetic modification comprises at least one nucleotide within the genomic coordinates chosen from: chr19:55115218-55115242; chr19:55115477-55115501; chr19:55115504-55115528; chr19:55115513-55115537; chr19:55115514-55115538; chr19:55115517-55115541; chr19:55115518-55115542; chr19:55115549-55115573; chr19:55115574-55115598; chr19:55115606-55115630; chr19:55115933-55115957; chr19:55116026-55116050; chr19:55116045-55116069; chr19:55116084-55116108; chr19:55115276-55115300; chr19:55115509-55115533; chr19:55115579-55115603; chr19:55115863-55115887; chr19:55115906-55115930; and chr19:55116006-55116030. In some embodiments, the genetic modification comprises at least 5 contiguous nucleotides within the genomic coordinates chosen from: chr19:55115218-55115242; chr19:55115477-55115501; chr19:55115504-55115528; chr19:55115513-55115537; chr19:55115514-55115538; chr19:55115517-55115541; chr19:55115518-55115542; chr19:55115549-55115573; chr19:55115574-55115598; chr19:55115606-55115630; chr19:55115933-55115957; chr19:55116026-55116050; chr19:55116045-55116069; chr19:55116084-55116108; chr19:55115276-55115300; chr19:55115509-55115533; chr19:55115579-55115603; chr19:55115863-55115887; chr19:55115906-55115930; and chr19:55116006-55116030. In some embodiments, the genetic modification comprises at least 10 contiguous nucleotides within the genomic coordinates chosen from: chr19:55115218-55115242; chr19:55115477-55115501; chr19:55115504-55115528; chr19:55115513-55115537; chr19:55115514-55115538; chr19:55115517-55115541; chr19:55115518-55115542; chr19:55115549-55115573; chr19:55115574-55115598; chr19:55115606-55115630; chr19:55115933-55115957; chr19:55116026-55116050; chr19:55116045-55116069; chr19:55116084-55116108; chr19:55115276-55115300; chr19:55115509-55115533; chr19:55115579-55115603; chr19:55115863-55115887; chr19:55115906-55115930; and chr19:55116006-55116030.

In some embodiments, the genetic modification comprises at least one nucleotide within the genomic coordinates chosen from: (a) chr19:55115518-55115542; chr19:55115517-55115541; chr19:55115504-55115528; chr19:55115514-55115538; chr19:55115477-55115501; chr19:55115276-55115300; chr19:55116026-55116050; chr19:55116084-55116108; chr19:55116045-55116069; chr19:55115933-55115957; chr19:55115218-55115242; and chr19:55115696-55115720; or (b) chr19:55115579-55115603; chr19:55116006-55116030; chr19:55115863-55115887; chr19:55116098-55116122; chr19:55115710-55115734; and chr19:55116014-55116038. In some embodiments, the genetic modification comprises at least 5 contiguous nucleotides within the genomic coordinates chosen from: (a) chr19:55115518-55115542; chr19:55115517-55115541; chr19:55115504-55115528; chr19:55115514-55115538; chr19:55115477-55115501; chr19:55115276-55115300; chr19:55116026-55116050; chr19:55116084-55116108; chr19:55116045-55116069; chr19:55115933-55115957; chr19:55115218-55115242; and chr19:55115696-55115720; or (b) chr19:55115579-55115603; chr19:55116006-55116030; chr19:55115863-55115887; chr19:55116098-55116122; chr19:55115710-55115734; and chr19:55116014-55116038. In some embodiments, the genetic modification comprises at least 10 contiguous nucleotides within the genomic coordinates chosen from: (a) chr19:55115518-55115542; chr19:55115517-55115541; chr19:55115504-55115528; chr19:55115514-55115538; chr19:55115477-55115501; chr19:55115276-55115300; chr19:55116026-55116050; chr19:55116084-55116108; chr19:55116045-55116069; chr19:55115933-55115957; chr19:55115218-55115242; and chr19:55115696-55115720; or (b) chr19:55115579-55115603; chr19:55116006-55116030; chr19:55115863-55115887; chr19:55116098-55116122; chr19:55115710-55115734; and chr19:55116014-55116038.

In some embodiments, the genetic modification comprises at least one indel, at least one C to T substitution, or at least one A to G substitution within the genomic coordinates chosen from: chr19:55115218-55115242; chr19:55115477-55115501; chr19:55115504-55115528; chr19:55115513-55115537; chr19:55115514-55115538; chr19:55115517-55115541; chr19:55115518-55115542; chr19:55115549-55115573; chr19:55115574-55115598; chr19:55115606-55115630; chr19:55115933-55115957; chr19:55116026-55116050; chr19:55116045-55116069; chr19:55116084-55116108; chr19:55115276-55115300; chr19:55115509-55115533; chr19:55115579-55115603; chr19:55115863-55115887; chr19:55115906-55115930; and chr19:55116006-55116030.

In some embodiments, the genetic modification comprises at least one indel, at least one C to T substitution, or at least one A to G substitution within the genomic coordinates chosen from: (a) chr19:55115518-55115542; chr19:55115517-55115541; chr19:55115504-55115528; chr19:55115514-55115538; chr19:55115477-55115501; chr19:55115276-55115300; chr19:55116026-55116050; chr19:55116084-55116108; chr19:55116045-55116069; chr19:55115933-55115957; chr19:55115218-55115242; and chr19:55115696-55115720; or (b) chr19:55115579-55115603; chr19:55116006-55116030; chr19:55115863-55115887; chr19:55116098-55116122; chr19:55115710-55115734; and chr19:55116014-55116038.

In some embodiments, the modification to AAVS1 comprises any one or more of an insertion, deletion, substitution, or deamination of at least one nucleotide in a target sequence. In some embodiments, the modification to AAVS1 comprises an insertion of 1, 2, 3, 4 or 5 or more nucleotides in a target sequence. In some embodiments, the modification to AAVS1 comprises a deletion of 1, 2, 3, 4 or 5 or more nucleotides in a target sequence. In other embodiments, the modification to AAVS1 comprises an insertion of 1, 2, 3, 4, 5, 6, 7, 8, 9, 10, 15, 20 or 25 or more nucleotides in a target sequence. In other embodiments, the modification to AAVS1 comprises a deletion of 1, 2, 3, 4, 5, 6, 7, 8, 9, 10, 15, 20 or 25 or more nucleotides in a target sequence. In some embodiments, the modification to AAVS1 comprises an indel, which is generally defined in the art as an insertion or deletion of less than 1000 base pairs (bp). In some embodiments, the modification to AAVS1 comprises an insertion of a donor nucleic acid in a target sequence. In some embodiments, the modification to AAVS1 is not transient.

In some embodiments, the methods and compositions disclosed herein modify the AAVS1 locus in a cell using an RNA-guided DNA binding agent (e.g., a Cas enzyme). In some embodiments, the RNA-guided DNA binding agent is Cas9. In some embodiments, the RNA-guided DNA binding agent cuts within the AAVS1 gene, wherein the AAVS1 guide RNA targets an AAVS1 genomic target sequence comprising at least 10 contiguous nucleotides chr19:55115151-55116209.

In some embodiments, the methods and compositions disclosed herein modify the AAVS1 locus in a cell, wherein the modification to AAVS1 comprises an insertion of an exogenous nucleic acid. In some embodiments, the exogenous nucleic acid is a protein-coding gene. The protein encoded by the exogenous nucleic acid may be expressed by the cell.

### H. Exemplary Cell Types

In some embodiments, methods and compositions disclosed herein genetically modify a cell. In some embodiments, the cell is an allogeneic cell. In some embodiments the cell is a human cell. In some embodiments the genetically modified cell is referred to as an engineered cell. An engineered cell refers to a cell (or progeny of a cell) comprising an engineered genetic modification, e.g. that has been contacted with a genomic editing system and genetically modified by the genomic editing system. The terms “engineered cell” and “genetically modified cell” are used interchangeably throughout. The engineered cell may be any of the exemplary cell types disclosed herein.

In some embodiments, the cell is an immune cell. As used herein, “immune cell” refers to a cell of the immune system, including e.g., a lymphocyte (e.g., T cell, B cell, natural killer cell (“NK cell”, and NKT cell, or iNKT cell)), monocyte, macrophage, mast cell, dendritic cell, or granulocyte (e.g., neutrophil, eosinophil, and basophil). In some embodiments, the cell is a primary immune cell. In some embodiments, the immune system cell may be selected from CD3+, CD4+ and CD8+ T cells, regulatory T cells (Tregs), B cells, NK cells, and dendritic cells (DC). In some embodiments, the immune cell is allogeneic.

In some embodiments, the cell is a lymphocyte. In some embodiments, the cell is an adaptive immune cell. In some embodiments, the cell is a T cell. In some embodiments, the cell is a B cell. In some embodiments, the cell is a NK cell. In some embodiments, the lymphocyte is allogeneic.

As used herein, a T cell can be defined as a cell that expresses a T cell receptor (“TCR” or “ap TCR” or “76 TCR”), however in some embodiments, the TCR of a T cell may be genetically modified to reduce its expression (e.g., by genetic modification to the TRAC or TRBC genes), therefore expression of the protein CD3 may be used as a marker to identify a T cell by standard flow cytometry methods. CD3 is a multi-subunit signaling complex that associates with the TCR. Thus, a T cell may be referred to as CD3+. In some embodiments, a T cell is a cell that expresses a CD3+ marker and either a CD4+ or CD8+ marker. In some embodiments, the T cell is allogeneic.

In some embodiments, the T cell expresses the glycoprotein CD8 and therefore is CD8+ by standard flow cytometry methods and may be referred to as a “cytotoxic” T cell. In some embodiments, the T cell expresses the glycoprotein CD4 and therefore is CD4+ by standard flow cytometry methods and may be referred to as a “helper” T cell. CD4+ T cells can differentiate into subsets and may be referred to as a Th1 cell, Th2 cell, Th9 cell, Th17 cell, Th22 cell, T regulatory (“Treg”) cell, or T follicular helper cells (“Tfh”). Each CD4+ subset releases specific cytokines that can have either proinflammatory or anti-inflammatory functions, survival or protective functions. A T cell may be isolated from a subject by CD4+ or CD8+ selection methods.

In some embodiments, the T cell is a memory T cell. In the body, a memory T cell has encountered antigen. A memory T cell can be located in the secondary lymphoid organs (central memory T cells) or in recently infected tissue (effector memory T cells). A memory T cell may be a CD8+ T cell. A memory T cell may be a CD4+ T cell.

As used herein, a “central memory T cell” can be defined as an antigen-experienced T cell, and for example, may expresses CD62L and CD45RO. A central memory T cell may be detected as CD62L+ and CD45RO+ by Central memory T cells also express CCR7, therefore may be detected as CCR7+ by standard flow cytometry methods.

As used herein, an “early stem-cell memory T cell” (or “Tscm”) can be defined as a T cell that expresses CD27 and CD45RA, and therefore is CD27+ and CD45RA+ by standard flow cytometry methods. A Tscm does not express the CD45 isoform CD45RO, therefore a Tscm will further be CD45RO− if stained for this isoform by standard flow cytometry methods. A CD45RO-CD27+ cell is therefore also an early stem-cell memory T cell. Tscm cells further express CD62L and CCR7, therefore may be detected as CD62L+ and CCR7+ by standard flow cytometry methods. Early stem-cell memory T cells have been shown to correlate with increased persistence and therapeutic efficacy of cell therapy products.

In some embodiments, the cell is a B cell. As used herein, a “B cell” can be defined as a cell that expresses CD19 or CD20, or B cell mature antigen (“BCMA”), and therefore a B cell is CD19+, or CD20+, or BCMA+ by standard flow cytometry methods. A B cell is further negative for CD3 and CD56 by standard flow cytometry methods. The B cell may be a plasma cell. The B cell may be a memory B cell. The B cell may be a naïve B cell. The B cell may be IgM+, or has a class-switched B cell receptor (e.g., IgG+, or IgA+). In some embodiments, the B cell is allogeneic.

In some embodiments, the cell is a mononuclear cell, such as from bone marrow or peripheral blood. In some embodiments, the cell is a peripheral blood mononuclear cell (“PBMC”). In some embodiments, the cell is a PBMC, e.g. a lymphocyte or monocyte. In some embodiments, the cell is a peripheral blood lymphocyte (“PBL”). In some embodiments, the mononuclear cell is allogeneic.

Cells used in ACT or tissue regenerative therapy are included, such as stem cells, progenitor cells, and primary cells. Stem cells, for example, include pluripotent stem cells (PSCs); induced pluripotent stem cells (iPSCs); embryonic stem cells (ESCs); mesenchymal stem cells (MSCs, e.g., isolated from bone marrow (BM), peripheral blood (PB), placenta, umbilical cord (UC) or adipose); hematopoietic stem cells (HSCs; e.g. isolated from BM or UC); neural stem cells (NSCs); tissue specific progenitor stem cells (TSPSCs); and limbal stem cells (LSCs). Progenitor and primary cells include mononuclear cells (MNCs, e.g., isolated from BM or PB); endothelial progenitor cells (EPCs, e.g. isolated from BM, PB, and UC); neural progenitor cells (NPCs); and tissue-specific primary cells or cells derived therefrom (TSCs) including chondrocytes, myocytes, and keratinocytes. Cells for organ or tissue transplantations such as islet cells, cardiomyocytes, thyroid cells, thymocytes, neuronal cells, skin cells, and retinal cells are also included.

In some embodiments, the cell is a human cell, such as a cell isolated from a human subject. In some embodiments, the cell is isolated from human donor PBMCs or leukopaks. In some embodiments, the cell is from a subject with a condition, disorder, or disease. In some embodiments, the cell is from a human donor with Epstein Barr Virus (“EBV”).

In some embodiments, the methods are carried out ex vivo. As used herein, “ex vivo” refers to an in vitro method wherein the cell is capable of being transferred into a subject, e.g. as an ACT therapy. In some embodiments, an ex vivo method is an in vitro method involving an ACT therapy cell or cell population.

In some embodiments, the cell is from a cell line. In some embodiments, the cell line is derived from a human subject. In some embodiments, the cell line is a lymphoblastoid cell line (“LCL”). The cell may be cryopreserved and thawed. The cell may not have been previously cryopreserved.

In some embodiments, the cell is from a cell bank. In some embodiments, the cell is genetically modified and then transferred into a cell bank. In some embodiments the cell is removed from a subject, genetically modified ex vivo, and transferred into a cell bank. In some embodiments, a genetically modified population of cells is transferred into a cell bank. In some embodiments, a genetically modified population of immune cells is transferred into a cell bank. In some embodiments, a genetically modified population of immune cells comprising a first and second subpopulations, wherein the first and second sub-populations have at least one common genetic modification and at least one different genetic modification are transferred into a cell bank.

### III. Details of the Genomic Editing Systems

In some embodiments, the genomic editing system is a CRISPR/Cas system, including e.g., a CRISPR guide RNA comprising a guide sequence and RNA-guided DNA binding agent, and described further herein. Further description of the CRISPR/Cas system methods and compositions for use therein are known in the art. See e.g., PCT/US2021/062922 filed Dec. 10, 2021, and U.S. Provisional Application No. 63/275,425 filed Nov. 3, 2021, the contents of each of which are hereby incorporated in their entireties.

### A. CRISPR Guide RNA

Provided herein are guide sequences useful for modifying a target sequence, e.g., using a guide RNA comprising a disclosed guide sequence with an RNA-guided DNA binding agent (e.g., a CRISPR/Cas system). Guide sequences targeting the HLA-A, TRAC, TRBC, and CIITA are shown in Tables 1-5 (SEQ ID NOs: 2-80, 101-120, 201-265, 301, 302, 304-576, and 601-774), as are the genomic coordinates that these guide RNAs target.

In some embodiments, a gRNA provided herein comprises a guide region (guide sequence) and a conserved region comprising a repeat/anti-repeat region, a hairpin 1 region, and a hairpin 2 region, wherein one or more of the repeat/anti-repeat region, the hairpin 1 region, and the hairpin 2 region are shortened. In some embodiments, the gRNA is an N. meningitidis Cas9 (NmeCas9) gRNA.

In some embodiments, the guide RNA comprises a modified sgRNA. In some embodiments, the sgRNA comprises any one of the modification pattern of the modified sgRNA sequences provided in Tables 1-5, 6, 7, and 7A-7B. In some embodiments, the conserved region comprises any one of modified conserved region Nme guide RNA motifs in Table 7B, and wherein the conserved region is 3′ of the guide region (guide sequence). In some embodiments, the conserved region comprises a modified sequence comprising any one of SEQ ID NOs: 1081-1089, and wherein the conserved region is 3′ of the guide region (guide sequence). In some embodiments, the guide RNA comprises a nucleotide sequence selected from any one of SEQ ID NOs: 904-909, 911, and 995-997, where the N's represent collectively any guide sequence disclosed herein, including the guide sequences provided in Tables 1-5. In certain embodiments, the N's represent collectively a guide sequence that is at least 80%, 85%, 90%, 95%, or 100% identical to or complementary to any one of the guide sequences provided in Tables 1-5. In certain embodiments, the N's represent collectively any one of the guide sequences provided in Tables 1-5. In certain embodiments, when the N's represent collectively a guide sequence, within (N)₂₀₋₂₅, each N of the (N)₂₀₋₂₅ may be independently modified, e.g., modified with a 2′—OMe modification, optionally further with a PS modification, particularly at 1, 2, or 3 terminal nucleotides. In certain embodiments, the (N)₂₀₋₂₅ has the following sequence and modification pattern: mN*mN*mN*mNmNNNmNmNNmNNmNNNNNmNNNNmNNN.

An exemplary conserved region of an NmeCas9 single guide RNA (Nme sgRNA) is shown in Table 6 (SEQ ID NO: 900). The first row shows the numbering of the nucleotides; the second row shows an exemplary sequence; and the third (and fourth) rows show the regions. “Shortened” with respect to an sgRNA means that its conserved region lacks at least one nucleotide shown in Table 6, as discussed in detail below.

In some embodiments, the guide RNA is a Nme sgRNA comprising a conserved portion comprising a repeat/anti-repeat region, a hairpin 1 region, and a hairpin 2 region, wherein one or more of the repeat/anti-repeat region, the hairpin 1 region, and the hairpin 2 region are shortened. In some embodiments, the sgRNA described herein further comprises a guide region and a conserved region, wherein the conserved region comprises one or more of: (a) a shortened repeat/anti-repeat region, wherein the shortened repeat/anti-repeat region lacks 2-24 nucleotides, wherein (i) one or more of nucleotides 37-48 and 53-64 is deleted and optionally one or more of nucleotides 37-64 is substituted relative to SEQ ID NO: 900; and (ii) nucleotide 36 is linked to nucleotide 65 by at least 2 nucleotides; or (b) a shortened hairpin 1 region, wherein the shortened hairpin 1 lacks 2-10, optionally 2-8 nucleotides, wherein one or more of nucleotides 82-86 and 91-95 is deleted and optionally one or more of positions 82-96 is substituted relative to SEQ ID NO: 900; and nucleotide 81 is linked to nucleotide 96 by at least 4 nucleotides; or (c) a shortened hairpin 2 region, wherein the shortened hairpin 2 lacks 2-18, optionally 2-16 nucleotides, wherein (i) one or more of nucleotides 113-121 and 126-134 is deleted and optionally one or more of nucleotides 113-134 is substituted relative to SEQ ID NO: 900; and (ii) nucleotide 112 is linked to nucleotide 135 by at least 4 nucleotides; wherein one or both nucleotides 144-145 are optionally deleted relative to SEQ ID NO: 900; and wherein at least 10 nucleotides are modified nucleotides.

In some embodiment, the gRNA disclosed herein is a sgRNA.

In some embodiments, in the guide sequence, nucleotides 1-4 are modified nucleotides. In some embodiments, in the guide sequence, nucleotides 5, 8, 9, 11, 13, 18, and 22 are modified nucleotides. In some embodiments, in the guide sequence, nucleotides 1-5, 8, 9, 11, 13, 18, and 22 are modified nucleotides. In some embodiments, the modified nucleotides are 2′—O-methyl (2′—O-Me) modified nucleotides. In some embodiments, in the guide sequence, nucleotide 1 is linked to nucleotide 2 by a phosphorothioate (PS) linkage, nucleotide 2 is linked to nucleotide 3 by a PS linkage, and/or nucleotide 3 is linked to nucleotide 4 by a PS linkage.

In some embodiments, one or both nucleotides 144-145 are deleted relative to SEQ ID NO: 900.

In some embodiments, at least 10 nucleotides of the conserved region are modified nucleotides.

In some embodiments, a repeat/anti-repeat region of a gRNA is a shortened repeat/anti-repeat region lacking 2-24 nucleotides, e.g., any of the repeat/anti-repeat regions indicated in the numbered embodiments above or Tables 1-6 or described elsewhere herein, which may be combined with any of the shortened hairpin 1 region or hairpin 2 region described herein, including but not limited to combinations indicated in the numbered embodiments above and represented in the sequences of Tables 1-6 or described elsewhere herein. In some embodiments, one or more of positions 49-52, 87-90, or 122-125 is substituted relative to SEQ ID NO: 900. In some embodiments, all of positions 49-52, 87-90, or 122-125 are substituted relative to SEQ ID NO: 900. In some embodiments, the 3′ tail provided in Tables 1-6 or described herein is deleted.

In some embodiments, the shortened repeat/anti-repeat region of the gRNA lacks 18 nucleotides. In some embodiments, the shortened repeat/anti-repeat region of the gRNA lacks 22 nucleotides.

In some embodiments, in the shortened repeat/anti-repeat region of the gRNA, nucleotide 36 is linked to nucleotide 65 by 6 nucleotides. In some embodiments, in the shortened repeat/anti-repeat region of the gRNA, nucleotide 36 is linked to nucleotide 65 by 7 nucleotides. In some embodiments, in the shortened repeat/anti-repeat region of the gRNA, nucleotide 36 is linked to nucleotide 65 by 8 nucleotides. In some embodiments, in the shortened repeat/anti-repeat region of the gRNA, nucleotide 36 is linked to nucleotide 65 by 9 nucleotides. In some embodiments, in the shortened repeat/anti-repeat region of the gRNA, nucleotide 36 is linked to nucleotide 65 by 10 nucleotides.

In some embodiments, in the shortened repeat/anti-repeat region of the gRNA, nucleotides 38-48 and 53-63 are deleted relative to SEQ ID NO: 900. In some embodiments, in the shortened repeat/anti-repeat region of the gRNA, nucleotides 38, 41-48, 53-60, and 63 are deleted relative to SEQ ID NO: 900.

In some embodiments, in the shortened repeat/anti-repeat region of the gRNA, nucleotide 36 is linked to nucleotide 65 by 6 nucleotides. In some embodiments, in the shortened repeat/anti-repeat region of the gRNA, nucleotides 38-48 and 53-63 are deleted relative to SEQ ID NO: 700, and nucleotide 36 is linked to nucleotide 65 by nucleotides 37, 49-52, and 64.

In some embodiments, in the shortened repeat/anti-repeat region of the gRNA, nucleotide 36 is linked to nucleotide 65 by 10 nucleotides. In some embodiments, in the shortened repeat/anti-repeat region of the gRNA, nucleotides 38, 41-48, 53-60, and 63 are deleted relative to SEQ ID NO: 900, and nucleotide 36 is linked to nucleotide 65 by nucleotides 37, 39, 40, 49-52, 61, 62, and 64.

In some embodiments, all of nucleotides 38-48 and nucleotides 53-63 of the upper stem of the shortened repeat/anti-repeat region are deleted relative to SEQ ID NO: 900.

In some embodiments, all of nucleotides 39-48 and nucleotides 53-62 of the upper stem of the shortened repeat/anti-repeat region are deleted relative to SEQ ID NO: 900, and nucleotides 38 and 63 is substituted.

In some embodiments, the shortened repeat/anti-repeat region has 14 modified nucleotides. In some embodiments, the shortened repeat/anti-repeat region has 15 modified nucleotides. In some embodiments, the shortened repeat/anti-repeat region has 16 modified nucleotides. In some embodiments, the shortened repeat/anti-repeat region has 17 modified nucleotides. In some embodiments, the shortened repeat/anti-repeat region has 18 modified nucleotides. In some embodiments, the shortened repeat/anti-repeat region has 19 modified nucleotides. In some embodiments, the shortened repeat/anti-repeat region has 20 modified nucleotides. In some embodiments, in the shortened repeat/anti-repeat region, nucleotides 25, 29, 30, 31, 32, 37, 49-52, 64, 65, 69, 70, and 73 are modified nucleotides. In some embodiments, the modified nucleotides are 2′—O-Me modified nucleotides.

In some embodiments, between the shortened repeat/anti-repeat region and the shortened hairpin 1 region, nucleotide 76 is linked to nucleotide 77 by a PS linkage.

In some embodiments, the shortened hairpin 1 region lacks 2 nucleotides. In some embodiments, the shortened hairpin 1 region lacks 21 nucleotides. In some embodiments, the shortened hairpin 1 region lacks 2 nucleotides, and nucleotides 86 and 91 are deleted relative to SEQ ID NO: 900. In some embodiments, the shortened hairpin 1 region lacks 2 nucleotides, and nucleotides 85 and 92 are deleted relative to SEQ ID NO: 900. In some embodiments, in the shortened hairpin 1 region, nucleotide 81 is linked to nucleotide 96 by 12 nucleotides. In some embodiments, in the shortened hairpin 1 region, nucleotide 81 is linked to nucleotide 96 by 12 nucleotides. In some embodiments, in the shortened hairpin 1 region, nucleotides 86 and 91 are deleted relative to SEQ ID NO: 900, and nucleotide 81 is linked to nucleotide 96 by nucleotides 82-85, 87-90, and 92-95. In some embodiments, in the shortened hairpin 1 region, nucleotides 85 and 92 are deleted relative to SEQ ID NO: 900, and nucleotide 81 is linked to nucleotide 96 by nucleotides 82-84, 86-91, and 93-95.

In some embodiments, the shortened hairpin 1 region has a duplex portion of 7 base paired nucleotides in length. In some embodiments, the shortened hairpin 1 region has a duplex portion of 8 base paired nucleotides in length.

In the stem of the shortened hairpin 1 region is seven base paired nucleotides in length. In some embodiments, nucleotides 85-86 and nucleotides 91-92 of the shortened hairpin 1 region are deleted.

In some embodiments, the shortened hairpin 1 region has 13 modified nucleotides. In some embodiments, in the shortened hairpin 1 region, nucleotides 80, 81, 83, 84, 85, 87-90, 92-94, and 99 are modified nucleotides. In some embodiments, the modified nucleotides are 2′—O-Me modified nucleotides.

In some embodiments, between the shortened hairpin 1 region and the shortened hairpin 2 region, nucleotide 101 is a modified nucleotide. In some embodiments, the modified nucleotide is a 2′—O-Me modified nucleotide.

In some embodiments, the shortened hairpin 2 lacks 18 nucleotides. In some embodiments, the shortened hairpin 2 has 24 nucleotides. In some embodiments, in the shortened hairpin 2 nucleotides 113-121 and 126-134 are deleted relative to SEQ ID NO: 900. In some embodiments, the shortened hairpin 2 lacks 18 nucleotides, and nucleotides 113-121 and 126-134 are deleted relative to SEQ ID NO: 900. In some embodiments, in the shortened hairpin 2 region, nucleotide 112 is linked to nucleotide 135 by 4 nucleotides. In some embodiments, in the shortened hairpin 2 region, nucleotides 113-121 and 126-134 are deleted relative to SEQ ID NO: 900 and nucleotide 112 is linked to nucleotide 135 by nucleotides 122-125. In some embodiments, in the shortened hairpin 2 region, nucleotides 112-120 and 127-135 are deleted relative to SEQ ID NO: 900. In some embodiments, the shortened hairpin 2 region lacks 18 nucleotides, and nucleotides 112-120 and 127-135 are deleted relative to SEQ ID NO: 900.

In some embodiments, the shortened repeat/anti-repeat region has a length of 28 nucleotides. In some embodiments, the shortened repeat/anti-repeat region has a length of 32 nucleotides.

In some embodiments, the upper stem of the shortened repeat/anti-repeat region comprises no more than one base pair. In some embodiments, the upper stem of the shortened repeat/anti-repeat region comprises no more than three base pairs.

In some embodiments, the shortened hairpin 2 region has 8 modified nucleotides. In some embodiments, the shortened hairpin 2 region has 9 modified nucleotides. In some embodiments, the shortened hairpin 2 region has 13 modified nucleotides. In some embodiments, in the shortened hairpin 2 region, nucleotides 104, 110, 111, 122-125, 142, and 143 are modified nucleotides. In some embodiments, in the shortened hairpin 2 region, nucleotides 104, 106-111, 122-125, 142, and 143 are modified nucleotides. In some embodiments, the modified nucleotides are 2′—O-Me modified nucleotides.

In some embodiments, in the shortened hairpin 2 region, nucleotide 141 is linked to nucleotide 142 by a PS linkage, and/or nucleotide 142 is linked to nucleotide 143 by a PS linkage.

In some embodiments, a guide RNA (gRNA) comprises a guide region and a conserved region, the conserved region comprising:
(a) a shortened repeat/anti-repeat region, wherein the shortened repeat/anti-repeat region lacks 18-22 nucleotides relative to SEQ ID NO: 900, wherein
   (i) nucleotides 38-48 and 53-63 are deleted; and
   (ii) nucleotide 36 is linked to nucleotide 65 by 6-10 nucleotides;
(b) a shortened hairpin 1 region, wherein the shortened hairpin 1 lacks 2 nucleotides, wherein nucleotides 86 and 91 are deleted or nucleotides 85 and 92 are deleted relative to SEQ ID NO: 900; and
(c) a shortened hairpin 2 region, wherein the shortened hairpin 2 lacks 18 nucleotides, wherein nucleotides 113-121 and 126-134 are deleted relative to SEQ ID NO: 900; and wherein nucleotides 144-145 are deleted relative to SEQ ID NO: 900; wherein at least 10 nucleotides are modified nucleotides.

In some embodiments, a guide RNA (gRNA) comprises a guide region and a conserved region, the conserved region comprising:
(a) a shortened repeat/anti-repeat region, wherein the shortened repeat/anti-repeat region lacks 18-22 nucleotides relative to SEQ ID NO: 900, wherein
   (i) nucleotides 38, 41-48, 53-60, and 63 are deleted; and
   (ii) nucleotide 36 is linked to nucleotide 65 by 6-10 nucleotides;
(b) a shortened hairpin 1 region, wherein the shortened hairpin 1 lacks 2 nucleotides, wherein nucleotides 86 and 91 are deleted or nucleotides 85 and 92 are deleted relative to SEQ ID NO: 700;
(c) a shortened hairpin 2 region, wherein the shortened hairpin 2 lacks 18 nucleotides, wherein nucleotides 113-121 and 126-134 are deleted relative to SEQ ID NO: 900; and wherein nucleotides 144-145 are deleted relative to SEQ ID NO: 900; wherein at least 10 nucleotides are modified nucleotides.

In some embodiments, a guide RNA (gRNA) is provided, the gRNA comprising a guide region and a conserved region, the conserved region comprising one or more of:
(a) a shortened repeat/anti-repeat region, wherein the shortened repeat/anti-repeat region lacks 18-22 nucleotides relative to SEQ ID NO: 900, wherein
   (i) nucleotides 37-48 and 53-64 are deleted; and
   (ii) nucleotide 36 is linked to nucleotide 65 by 6-10 nucleotides; or
(b) a shortened hairpin 1 region, wherein the shortened hairpin 1 lacks 2 nucleotides, wherein nucleotides 86 and 91 are deleted or nucleotides 85 and 92 are deleted relative to SEQ ID NO: 900; or
(c) a shortened hairpin 2 region, wherein the shortened hairpin 2 lacks 18 nucleotides, wherein nucleotides 113-121 and 126-134 are deleted relative to SEQ ID NO: 900; and
wherein nucleotides 144-145 are deleted relative to SEQ ID NO: 900;
wherein at least 10 nucleotides are modified nucleotides.

In further embodiments, the shortened repeat/anti-repeat region, wherein the shortened repeat/anti-repeat region lacks 22 nucleotides relative to SEQ ID NO: 900. In further embodiments, nucleotide 36 is linked to nucleotide 65 by a sequence comprising the nucleotide sequence UGAAAC. In further embodiments, the nucleotide 36 is linked to nucleotide 65 by 10 nucleotides. In further embodiments, the nucleotide 36 is linked to nucleotide 65 by a sequence comprising the nucleotide sequence UUCGAAAGAC (SEQ ID NO: 3011).

In some embodiments, the guide RNA (gRNA) of the previous embodiment comprising a guide region and a conserved region, the conserved region comprising:
(a) a shortened repeat/anti-repeat region, wherein the shortened repeat/anti-repeat region lacks 18-22 nucleotides, wherein
   (i) nucleotides 37-48 and 53-64 are deleted relative to SEQ ID NO: 900; and
   (ii) nucleotide 36 is linked to nucleotide 65 by 6-10 nucleotides;
(b) a shortened hairpin 1 region, wherein the shortened hairpin 1 lacks 2 nucleotides relative to SEQ ID NO: 900, wherein nucleotides 86 and 91 are deleted or nucleotides 85 and 92 are deleted;
(c) a shortened hairpin 2 region, wherein the shortened hairpin 2 lacks 18 nucleotides, wherein nucleotides 113-121 and 126-134 are deleted relative to SEQ ID NO: 900; and
(d) wherein nucleotides 144-145 are deleted relative to SEQ ID NO: 900; wherein at least 10 nucleotides are modified nucleotides.

In further embodiments, the shortened repeat/anti-repeat region, wherein the shortened repeat/anti-repeat region lacks 22 nucleotides relative to SEQ ID NO: 900. In further embodiments, nucleotide 36 is linked to nucleotide 65 by a sequence comprising the nucleotide sequence UGAAAC. In further embodiments, the nucleotide 36 is linked to nucleotide 65 by 10 nucleotides. In further embodiments, the nucleotide 36 is linked to nucleotide 65 by a sequence comprising the nucleotide sequence UUCGAAAGAC (SEQ ID NO: 3011).

In some embodiments, a guide RNA (gRNA) is provided, the gRNA comprising: a guide sequence comprising:
2′—O-Me modified nucleotides at the first four nucleotides 1-4;
PS linkages between nucleotides 1-2, 2-3, and 3-4; and
2′—O-Me modified nucleotides at nucleotides 5, 8, 9, 11, 13, 18, and 22 of the guide sequence;
   a shortened repeat/anti-repeat region, wherein nucleotides 38-48 and 53-63 are deleted relative to SEQ ID NO: 900, comprising:
2′—O-Me modified nucleotides at nucleotides 25, 29, 30, 31, 32, 37, 49-52, 64, 65, 69, 70, and 73;
   a PS linkage between nucleotides 76-77 between the shortened repeat/anti-repeat region and the shortened hairpin 1 region;
   a shortened hairpin 1 region, wherein nucleotides 86 and 91 are deleted relative to SEQ ID NO: 900, comprising:
2′—O-Me modified nucleotides at nucleotides 80, 81, 83, 84, 85, 87-90, 92-94, and 99; 2′—O-Me modified nucleotide at nucleotide 101 between the shortened hairpin 1 region and the shortened hairpin 2 region;
   a shortened hairpin 2 region, wherein nucleotides 112-120 and 127-135 are deleted relative to SEQ ID NO: 900, comprising:
2′—O-Me modified nucleotides at nucleotides 104, 110, 111, 122-125, 142, and 143,
PS linkages between nucleotides 141-142 and 142-143,
   wherein one or both nucleotides 144-145 are optionally deleted relative to SEQ ID NO: 900.

In some embodiments, a guide RNA (gRNA) is provided, the gRNA comprising: a guide sequence comprising:
2′—O-Me modified nucleotides at the first four nucleotides 1-4;
PS linkages between nucleotides 1-2, 2-3, and 3-4; and
2′—O-Me modified nucleotides at nucleotides 5, 8, 9, 11, 13, 18, and 22 of the guide sequence;
   a shortened repeat/anti-repeat region, wherein nucleotides 38-48 and 53-63 are deleted relative to SEQ ID NO: 900, comprising:
2′—O-Me modified nucleotides at nucleotides 25, 29, 30, 31, 32, 37, 49-52, 64, 65, 69, 70, and 73;
   a shortened hairpin 1 region, wherein nucleotides 86 and 91 are deleted relative to SEQ ID NO: 900, comprising:
2′—O-Me modified nucleotides at nucleotides 80, 81, 83, 84, 85, 87-90, 92-94, and 99;
   2′—O-Me modified nucleotide at nucleotide 101 between the shortened hairpin 1 region and the shortened hairpin 2 region;
   a shortened hairpin 2 region, wherein nucleotides 112-120 and 127-135 are deleted relative to SEQ ID NO: 900, comprising:
2′—O-Me modified nucleotides at nucleotides 104, 110, 111, 122-125, 142, and 143,
PS linkages between nucleotides 141-142 and 142-143,
   wherein one or both nucleotides 144-145 are optionally deleted relative to SEQ ID NO: 900.

In some embodiments, a guide RNA (gRNA) is provided, the gRNA comprising: a guide sequence comprising:
2′—O-Me modified nucleotides at the first four nucleotides 1-4;
PS linkages between nucleotides 1-2, 2-3, and 3-4; and
2′—O-Me modified nucleotides at nucleotides 5, 8, 9, 11, 13, 18, and 22 of the guide sequence;
   a shortened repeat/anti-repeat region, wherein nucleotides 38-48 and 53-63 are deleted relative to SEQ ID NO: 900, comprising:
2′—O-Me modified nucleotides at nucleotides 25, 29, 30, 31, 32, 37, 49-52, 64, 65, 69, 70, and 73;
   a PS linkage between nucleotides 76-77 between the shortened repeat/anti-repeat region and the shortened hairpin 1 region;
   a shortened hairpin 1 region, wherein nucleotides 86 and 91 are deleted relative to SEQ ID NO: 900, comprising:
2′—O-Me modified nucleotides at nucleotides 80, 81, 83, 84, 85, 87-90, 92-94, and 99;
   2′—O-Me modified nucleotide at nucleotide 101 between the shortened hairpin 1 region and the shortened hairpin 2 region;
   a shortened hairpin 2 region, wherein nucleotides 112-120 and 127-135 are deleted relative to SEQ ID NO: 900, comprising:
2′—O-Me modified nucleotides at nucleotides 104, 106-111, 122-125, 142, and 143,
PS linkages between nucleotides 141-142 and 142-143,
   wherein one or both nucleotides 144-145 are optionally deleted relative to SEQ ID NO: 900.

In some embodiments, a guide RNA (gRNA) is provided, the gRNA comprising: a guide sequence comprising:
2′—O-Me modified nucleotides at the first four nucleotides 1-4;
PS linkages between nucleotides 1-2, 2-3, and 3-4; and
2′—O-Me modified nucleotides at nucleotides 5, 8, 9, 11, 13, 18, and 22 of the guide sequence;
   a shortened repeat/anti-repeat region, wherein nucleotides 38-48 and 53-63 are deleted relative to SEQ ID NO: 900, comprising:
2′—O-Me modified nucleotides at nucleotides 25, 29, 30, 31, 32, 37, 49-52, 64, 65, 69, 70, and 73;
   a shortened hairpin 1 region, wherein nucleotides 86 and 91 are deleted relative to SEQ ID NO: 900, comprising:
2′—O-Me modified nucleotides at nucleotides 80, 81, 83, 84, 85, 87-90, 92-94, and 99;
   2′—O-Me modified nucleotide at nucleotide 101 between the shortened hairpin 1 region and the shortened hairpin 2 region;
   a shortened hairpin 2 region, wherein nucleotides 112-120 and 127-135 are deleted relative to SEQ ID NO: 900, comprising:
2′—O-Me modified nucleotides at nucleotides 104, 106-111, 122-125, 142, and 143,
PS linkages between nucleotides 141-142 and 142-143,
   wherein one or both nucleotides 144-145 are optionally deleted relative to SEQ ID NO: 900.

In some embodiments, the NmeCas9 sgRNA comprises any one of the Nme Cas9 guide sequences disclosed herein and additional nucleotides to form a crRNA, e.g., with the following exemplary scaffold nucleotide sequence following the guide sequence at its 3′ end:

(SEQ ID NO: 899)
GUUGUAGCUCCCUUUCUCAUUUCGGAAACGAAAUGAGAACCGUUGCUACA
AUAAGGCCGUCUGAAAAGAUGUGCCGCAACGCUCUGCCCCUUAAAGCUUC
UGCUUUAAGGGGCAUCGUUUA.

In some embodiments, the NmeCas9 sgRNA comprises any one of the guide sequences disclosed herein and additional nucleotides to form a crRNA with the following nucleotide sequence following the guide sequence at its 3′ end:

(SEQ ID NO: 901)
(N)₂₀₋₂₅GUUGUAGCUCCCUGAAACCGUUGCUACAAUAAGGCCGUCGAA
AAGAUGUGCCGCAACGCUCUGCCUUCUGGCAUCGUU;
(SEQ ID NO: 902)
(N)₂₀₋₂₅GUUGUAGCUCCCUGAAACCGUUGCUACAAUAAGGCCGUCGAA
AGAUGUGCCGCAACGCUCUGCCUUCUGGCAUCGUUUAUU;
(SEQ ID NO: 903)
(N)₂₀₋₂₅GUUGUAGCUCCCUGGAAACCCGUUGCUACAAUAAGGCCGUCG
AAAGAUGUGCCGCAACGCUCUGCCUUCUGGCAUCGUUUAUU

where A, C, G, U, and N are adenine, cytosine, guanine, uracil, and any ribonucleotide, respectively, unless otherwise indicated. In some embodiments, N equals 24. In some embodiments, N equals 25.

In some embodiments, the sgRNA comprises a conserved region comprising one of the following sequences in 5′ to 3′ orientation:

(SEQ ID NO: 906)
GUUGmUmAmGmCUCCCmUmGmAmAmAmCmCGUUmGmCUAmCAAU*AAGmG
mCCmGmUmCmGmAmAmAmGmAmUGUGCmCGCmAmAmCmGCUCUmGmCCmU
mUmCmUGmGCmAmUC*mG*mU*mU;
or
(SEQ ID NO: 1082)
mGUUGmUmAmGmCUCCCmUmGmAmAmAmCmCGUUmGmCUAmCAAU*AAGm
GmCCmGmUmCmGmAmAmAmGmAmUGUGCmCGmCAAmCGCUCUmGmCCmUm
UmCmUGGCAUCG*mU*mU;

or any one of SEQ ID NOs: 1081-1089, where A, C, G, U, and N are adenine, cytosine, guanine, uracil, and any ribonucleotide, respectively, unless otherwise indicated. An m is indicative of a 2′O-methyl modification, and an * is indicative of a phosphorothioate linkage between the nucleotides.

In certain embodiments, the guide sequence is 20-25 nucleotides in length ((N)20-25), wherein each nucleotide may be independently modified. In certain embodiments, each of nucleotides 1-3 of the 5′ end of the guide is independently modified. In certain embodiments, each of nucleotides 1-3 of the 5′ end of the guide is independently modified with a 2′—OMe modification. In certain embodiments, each of nucleotides 1-3 of the 5′ end of the guide is independently modified with a phosphorothioate linkage to the adjacent nucleotide residue. In certain embodiments, each of nucleotides 1-3 of the 5′ end of the guide is independently modified with a 2′—OMe modification and a phosphorothioate linkage to the adjacent nucleotide residue.

In certain embodiments, sgRNA, such as an sgRNA comprising Exemplary NmeCas9 sgRNA, further includes a 3′ tail, e.g., a 3′ tail of 1, 2, 3, 4, or more nucleotides. In certain embodiments, the tail includes one or more modified nucleotides. In certain embodiments, the modified nucleotide is selected from a 2′—O-methyl (2′—OMe) modified nucleotide, a 2′—O-(2-methoxyethyl) (2′—O-moe) modified nucleotide, a 2′-fluoro (2′-F) modified nucleotide, a phosphorothioate (PS) linkage between nucleotides, and an inverted abasic modified nucleotide, or a combination thereof. In certain embodiments, the modified nucleotide includes a 2′—OMe modified nucleotide. In certain embodiments, the modified nucleotide includes a PS linkage between nucleotides. In certain embodiments, the modified nucleotide includes a 2′—OMe modified nucleotide and a PS linkage between nucleotides.

In some embodiments, the guide RNA is a chemically modified guide RNA. In some embodiments, the guide RNA is a chemically modified single guide RNA. The chemically modified guide RNAs may comprise one or more of the modifications as shown in Tables 1-5. The chemically modified guide RNAs may comprise one or more of modified nucleotides of any one of SEQ ID NOs: 904-909, 911, 995-997, and 1081-1089.

In some embodiments, the guide RNA is a sgRNA comprising the modification pattern shown in any one of SEQ ID NO: 904-909, 911, 995-997, and 1081-1089.

In some embodiments, the guide RNA comprises a sgRNA comprising the modification pattern shown in SEQ ID NO: 907. In some embodiments, the guide RNA comprises a sgRNA comprising the modified nucleotides of SEQ ID NO: 907, including a guide sequence disclosed herein. In some embodiments, the guide RNA is a sgRNA comprising a sequence of SEQ ID NO: 907 or a sequence that is at least 99%, 98%, 97%, 96%, 95%, 94%, 93%, 92%, 91%, or 90% identical to SEQ ID NO: 907. In some embodiments, the guide RNA comprises a sgRNA comprising the modification pattern shown in SEQ ID NO: 995. In some embodiments, the guide RNA comprises a sgRNA comprising the modified nucleotides of SEQ ID NO: 995, including a guide sequence disclosed herein. In some embodiments, the guide RNA is a sgRNA comprising a sequence of SEQ ID NO: 995 or a sequence that is at least 99%, 98%, 97%, 96%, 95%, 94%, 93%, 92%, 91%, or 90% identical to SEQ ID NO: 995. In some embodiments, the guide RNA comprises a sgRNA comprising the modification pattern shown in SEQ ID NO: 996. In some embodiments, the guide RNA comprises a sgRNA comprising the modified nucleotides of SEQ ID NO: 996, including a guide sequence disclosed herein. In some embodiments, the guide RNA is a sgRNA comprising a sequence of SEQ ID NO: 996 or a sequence that is at least 99%, 98%, 97%, 96%, 95%, 94%, 93%, 92%, 91%, or 90% identical to SEQ ID NO: 996. In some embodiments, the guide RNA comprises a sgRNA comprising the modification pattern shown in SEQ ID NO: 997. In some embodiments, the guide RNA comprises a sgRNA comprising the modified nucleotides of SEQ ID NO: 997, including a guide sequence disclosed herein. In some embodiments, the guide RNA is a sgRNA comprising a sequence of SEQ ID NO: 997 or a sequence that is at least 99%, 98%, 97%, 96%, 95%, 94%, 93%, 92%, 91%, or 90% identical to SEQ ID NO: 997.

In some embodiments, the guide RNA comprises a sgRNA comprising the modification pattern shown in SEQ ID NO: 1082. In some embodiments, the guide RNA comprises a sgRNA comprising the modified nucleotides of SEQ ID NO: 1082, including a guide sequence disclosed herein. In some embodiments, the guide RNA is a sgRNA comprising a sequence of SEQ ID NO: 1082 or a sequence that is at least 99%, 98%, 97%, 96%, 95%, 94%, 93%, 92%, 91%, or 90% identical to SEQ ID NO: 1082. In some embodiments, the guide RNA comprises a sgRNA comprising the modification pattern shown in SEQ ID NO: 1083. In some embodiments, the guide RNA comprises a sgRNA comprising the modified nucleotides of SEQ ID NO: 1083, including a guide sequence disclosed herein. In some embodiments, the guide RNA is a sgRNA comprising a sequence of SEQ ID NO: 1083 or a sequence that is at least 99%, 98%, 97%, 96%, 95%, 94%, 93%, 92%, 91%, or 90% identical to SEQ ID NO: 1083. In some embodiments, the guide RNA comprises a sgRNA comprising the modification pattern shown in SEQ ID NO: 1084. In some embodiments, the guide RNA comprises a sgRNA comprising the modified nucleotides of SEQ ID NO: 1084, including a guide sequence disclosed herein. In some embodiments, the guide RNA is a sgRNA comprising a sequence of SEQ ID NO: 1084 or a sequence that is at least 99%, 98%, 97%, 96%, 95%, 94%, 93%, 92%, 91%, or 90% identical to SEQ ID NO: 1084. In some embodiments, the guide RNA comprises a sgRNA comprising the modification pattern shown in SEQ ID NO: 1085. In some embodiments, the guide RNA comprises a sgRNA comprising the modified nucleotides of SEQ ID NO: 1085, including a guide sequence disclosed herein. In some embodiments, the guide RNA is a sgRNA comprising a sequence of SEQ ID NO: 1085 or a sequence that is at least 99%, 98%, 97%, 96%, 95%, 94%, 93%, 92%, 91%, or 90% identical to SEQ ID NO: 1085.

The guide RNA may further comprise a trRNA. In each composition and method embodiment described herein, the crRNA and trRNA may be associated as a single RNA (sgRNA) or may be on separate RNAs (dgRNA). In the context of sgRNAs, the crRNA and trRNA components may be covalently linked, e.g., via a phosphodiester bond or other covalent bond. In some embodiments, a crRNA or trRNA sequence may be referred to as a “scaffold” or “conserved portion” of a guide RNA.

In each of the compositions, use, and method embodiments described herein, the guide RNA may comprise two RNA molecules as a “dual guide RNA” or “dgRNA.” The dgRNA comprises a first RNA molecule comprising a crRNA comprising, e.g., a guide sequence shown in Tables 1-5, and a second RNA molecule comprising a trRNA. The first and second RNA molecules may not be covalently linked, but may form an RNA duplex via the base pairing between portions of the crRNA and the trRNA.

In each of the composition, use, and method embodiments described herein, the guide RNA may comprise a single RNA molecule as a “single guide RNA” or “sgRNA”. The sgRNA may comprise a crRNA (or a portion thereof) comprising a guide sequence shown in Table 1, covalently linked to a trRNA. The sgRNA may comprise 20, 21, 22, 23, or 24 contiguous nucleotides of a guide sequence shown in Tables 1-5. In some embodiments, the crRNA and the trRNA are covalently linked via a linker. In some embodiments, the sgRNA forms a stem-loop structure via the base pairing between portions of the crRNA and the trRNA. In some embodiments, the crRNA and the trRNA are covalently linked via one or more bonds that are not a phosphodiester bond.

In some embodiments, the trRNA may comprise all or a portion of a trRNA sequence derived from a naturally-occurring CRISPR/Cas system. In some embodiments, the trRNA comprises a truncated or modified wild type trRNA. The length of the trRNA depends on the CRISPR/Cas system used. In some embodiments, the trRNA comprises or consists of 55, 60, 65, 70, 75, 80, 85, 90, 100, or more than 100 nucleotides. In some embodiments, the trRNA may comprise certain secondary structures, such as, for example, one or more hairpin or stem-loop structures, or one or more bulge structures.

In some embodiments, a composition comprising one or more guide RNAs comprising a guide sequence of any one in Tables 1-5 is provided.

In one aspect, a composition comprising a guide RNA that comprises a guide sequence that is at least 90% or 95% identical to any of the nucleic acids in Tables 1-5 is provided.

In other embodiments, a composition is provided that comprises at least one, e.g., at least two gRNAs comprising guide sequences selected from any two or more of the guide sequences shown in Tables 1-5. In some embodiments, the composition comprises at least two gRNAs that each comprise a guide sequence at least 90% or 95% identical to any of the nucleic acids shown in Tables 1-5.

In some embodiments, the guide RNA compositions of the present invention are designed to recognize (e.g., hybridize to) a target sequence in HLA-A. For example, the HLA-A target sequence may be recognized and cleaved by a provided Cas cleavase comprising a guide RNA. In some embodiments, an RNA-guided DNA binding agent, such as a Cas cleavase, may be directed by a guide RNA to a target sequence in HLA-A, where the guide sequence of the guide RNA hybridizes with the target sequence and the RNA-guided DNA binding agent, such as a Cas cleavase, cleaves the target sequence.

In some embodiments, the guide RNA compositions of the present invention are designed to recognize (or hybridize to) a target sequence in TRAC. For example, the TRAC target sequence may be recognized and cleaved by a provided Cas cleavase comprising a guide RNA. In some embodiments, an RNA-guided DNA binding agent, such as a Cas cleavase, may be directed by a guide RNA to a target sequence in TRAC, where the guide sequence of the guide RNA hybridizes with the target sequence and the RNA-guided DNA binding agent, such as a Cas cleavase, cleaves the target sequence.

In some embodiments, the guide RNA compositions of the present invention are designed to recognize (e.g., hybridize to) a target sequence in TRBC1. For example, the TRBC1 target sequence may be recognized and cleaved by a provided Cas cleavase comprising a guide RNA. In some embodiments, an RNA-guided DNA binding agent, such as a Cas cleavase, may be directed by a guide RNA to a target sequence in TRBC1, where the guide sequence of the guide RNA hybridizes with the target sequence and the RNA-guided DNA binding agent, such as a Cas cleavase, cleaves the target sequence.

In some embodiments, the guide RNA compositions of the present invention are designed to recognize (e.g., hybridize to) a target sequence in TRBC2. For example, the TRBC2 target sequence may be recognized and cleaved by a provided Cas cleavase comprising a guide RNA. In some embodiments, an RNA-guided DNA binding agent, such as a Cas cleavase, may be directed by a guide RNA to a target sequence in TRBC2, where the guide sequence of the guide RNA hybridizes with the target sequence and the RNA-guided DNA binding agent, such as a Cas cleavase, cleaves the target sequence.

In some embodiments, the guide RNA compositions of the present invention are designed to recognize (e.g., hybridize to) a target sequence in CIITA. For example, the CIITA target sequence may be recognized and cleaved by a provided Cas cleavase comprising a guide RNA. In some embodiments, an RNA-guided DNA binding agent, such as a Cas cleavase, may be directed by a guide RNA to a target sequence in CIITA, where the guide sequence of the guide RNA hybridizes with the target sequence and the RNA-guided DNA binding agent, such as a Cas cleavase, cleaves the target sequence.

In some embodiments, the guide RNA compositions of the present invention are designed to recognize (e.g., hybridize to) a target sequence in AAVS1. For example, the AAVS1 target sequence may be recognized and cleaved by a provided Cas cleavase comprising a guide RNA. In some embodiments, an RNA-guided DNA binding agent, such as a Cas cleavase, may be directed by a guide RNA to a target sequence in AAVS1, where the guide sequence of the guide RNA hybridizes with the target sequence and the RNA-guided DNA binding agent, such as a Cas cleavase, cleaves the target sequence.

In some embodiments, the selection of the one or more guide RNAs is determined based on target sequences within HLA-A, TRAC, TRBC, CIITA, or AAVS1. In some embodiments, the compositions comprising one or more guide sequences comprise a guide sequence that is complementary to the corresponding genomic region shown in Tables 1-5, according to coordinates from human reference genome hg38. Guide sequences of further embodiments may be complementary to sequences in the close vicinity of the genomic coordinate listed in any of the Tables 1-5 within HLA-A, TRAC, TRBC, CIITA, or AAVS1. For example, guide sequences of further embodiments may be complementary to sequences that comprise 10 contiguous nucleotides±10 nucleotides of a genomic coordinate listed in Tables 1-5.

Without being bound by any particular theory, modifications (e.g., frameshift mutations resulting from indels occurring as a result of a nuclease-mediated DSB) in certain regions of the target gene may be less tolerable than mutations in other regions, thus the location of a DSB is an important factor in the amount or type of protein knockdown that may result. In some embodiments, a gRNA complementary or having complementarity to a target sequence within the target gene used to direct an RNA-guided DNA binding agent to a particular location in the target gene.

In some embodiments, the Nme guide sequence is at least 90% or 95% or 100% identical to the reverse complement of a target sequence present in an HLA-A, TRAC, TRBC, CIITA, or AAVS1 gene. In some embodiments, the target sequence may be complementary to the guide sequence of the guide RNA. In some embodiments, the degree of complementarity or identity between a guide sequence of an Nme guide RNA and its corresponding target sequence is at least 80%, 85%, 90% or 95%; or 100%. In some embodiments, the target sequence and the guide sequence of the gRNA may be 100% complementary or identical.

In some embodiments, the target sequence and the guide sequence of the Nme gRNA may contain at least one mismatch. For example, the target sequence and the guide sequence of the gRNA may contain 1 or 2, less preferably 3, or 4 mismatches, where the total length of the guide sequence is 24 nucleotides. In some embodiments, the target sequence and the guide sequence of the gRNA may contain 1-2 mismatches, where the guide sequence is 24 nucleotides.

In some embodiments, the Nme guide sequence comprises a sequence of at least 21, 22, 23 or 24 contiguous nucleotides of a sequence selected from SEQ ID NOs: 2-80, 101-120, 201-265, 301, 302, 304-576, and 601-774.

In some embodiments, a composition or formulation disclosed herein comprises an mRNA comprising an open reading frame (ORF) encoding an RNA-guided DNA binding agent, such as a Cas nuclease as described herein. In some embodiments, an mRNA comprising an ORF encoding an RNA-guided DNA binding agent, such as a Cas nuclease, is provided, used, or administered.

In some embodiments, the gRNA is chemically modified. A gRNA comprising one or more modified nucleosides or nucleotides is called a “modified” gRNA or “chemically modified” gRNA, to describe the presence of one or more non-naturally or naturally occurring components or configurations that are used instead of or in addition to the canonical A, G, C, and U residues. Modified nucleosides and nucleotides can include one or more of: (i) alteration, e.g., replacement, of one or both of the non-linking phosphate oxygens or of one or more of the linking phosphate oxygens in the phosphodiester backbone linkage (an exemplary backbone modification); (ii) alteration, e.g., replacement, of a constituent of the ribose sugar, e.g., of the 2′ hydroxyl on the ribose sugar (an exemplary sugar modification); (iii) wholesale replacement of the phosphate moiety with “dephospho” linkers (an exemplary backbone modification); (iv) modification or replacement of a naturally occurring nucleobase, including with a non-canonical nucleobase (an exemplary base modification); (v) replacement or modification of the ribose-phosphate backbone (an exemplary backbone modification); (vi) modification of the 3′ end or 5′ end of the oligonucleotide, e.g., removal, modification or replacement of a terminal phosphate group or conjugation of a moiety, cap or linker (such 3′ or 5′ cap modifications may comprise a sugar or backbone modification); and (vii) modification or replacement of the sugar (an exemplary sugar modification).

Chemical modifications such as those listed above can be combined to provide modified gRNAs comprising nucleosides and nucleotides (collectively “residues”) that can have two, three, four, or more modifications. For example, a modified residue can have a modified sugar and a modified nucleobase. In some embodiments, every base of a gRNA is modified, e.g., all bases have a modified phosphate group, such as a phosphorothioate group. In certain embodiments, all, or substantially all, of the phosphate groups of an gRNA molecule are replaced with phosphorothioate groups. In some embodiments, modified gRNAs comprise at least one modified residue at or near the 5′ end of the RNA. In some embodiments, modified gRNAs comprise at least one modified residue at or near the 3′ end of the RNA.

In some embodiments, the gRNA comprises one, two, three or more modified residues. In some embodiments, at least 5% (e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%) of the positions in a modified gRNA are modified nucleosides or nucleotides.

In some embodiments of a backbone modification, the phosphate group of a modified residue can be modified by replacing one or more of the oxygens with a different substituent. Further, the modified residue, e.g., modified residue present in a modified nucleic acid, can include the wholesale replacement of an unmodified phosphate moiety with a modified phosphate group as described herein. In some embodiments, the backbone modification of the phosphate backbone can include alterations that result in either an uncharged linker or a charged linker with unsymmetrical charge distribution.

Examples of modified phosphate groups include phosphorothioate, phosphoroselenates, borano phosphates, borano phosphate esters, hydrogen phosphonates, phosphoroamidates, alkyl or aryl phosphonates and phosphotriesters.

Scaffolds that can mimic nucleic acids can also be constructed wherein the phosphate linker and ribose sugar are replaced by nuclease resistant nucleoside or nucleotide surrogates. Such modifications may comprise backbone and sugar modifications. In some embodiments, the nucleobases can be tethered by a surrogate backbone. Examples can include, without limitation, the morpholino, cyclobutyl, pyrrolidine and peptide nucleic acid (PNA) nucleoside surrogates. The modified nucleosides and modified nucleotides can include one or more modifications to the sugar group, i.e. at sugar modification. For example, the 2′ hydroxyl group (OH) can be modified, e.g. replaced with a number of different “oxy” or “deoxy” substituents. In some embodiments, modifications to the 2′ hydroxyl group can enhance the stability of the nucleic acid since the hydroxyl can no longer be deprotonated to form a 2′-alkoxide ion. Examples of 2′ hydroxyl group modifications can include alkoxy or aryloxy (OR, wherein “R” can be, e.g., alkyl, cycloalkyl, aryl, aralkyl, heteroaryl or a sugar); polyethyleneglycols (PEG), O(CH₂CH₂O)ₙCH₂CH₂OR wherein R can be, e.g., H or optionally substituted alkyl, and n can be an integer from 0 to 20. In some embodiments, the 2′ hydroxyl group modification can be 2′—O-Me. In some embodiments, the 2′ hydroxyl group modification can be a 2′-fluoro modification, which replaces the 2′ hydroxyl group with a fluoride. In some embodiments, the 2′ hydroxyl group modification can include “locked” nucleic acids (LNA) in which the 2′ hydroxyl can be connected, e.g., by a C₁₋₆ alkylene or C₁₋₆ heteroalkylene bridge, to the 4′ carbon of the same ribose sugar, where exemplary bridges can include methylene, propylene, ether, or amino bridges. In some embodiments, the 2′ hydroxyl group modification can included “unlocked” nucleic acids (UNA) in which the ribose ring lacks the C2′—C3′ bond. In some embodiments, the 2′ hydroxyl group modification can include the methoxyethyl group (MOE), (OCH₂CH₂OCH₃, e.g., a PEG derivative). [00708]“Deoxy” 2′ modifications can include hydrogen (i.e. deoxyribose sugars, e.g., at the overhang portions of partially dsRNA); halo (e.g., bromo, chloro, fluoro, or iodo); amino (wherein amino can be, e.g., NH₂; alkylamino, dialkylamino, heterocyclyl, arylamino, diarylamino, heteroarylamino, diheteroarylamino, or amino acid); NH(CH₂CH₂NH)ₙCH₂CH₂— amino (wherein amino can be, e.g., as described herein), —NHC(O)R (wherein R can be, e.g., alkyl, cycloalkyl, aryl, aralkyl, heteroaryl or sugar), cyano; mercapto; alkyl-thio-alkyl; thioalkoxy; and alkyl, cycloalkyl, aryl, alkenyl and alkynyl, which may be optionally substituted with e.g., an amino as described herein.

The sugar modification can comprise a sugar group which may also contain one or more carbons that possess the opposite stereochemical configuration than that of the corresponding carbon in ribose. Thus, a modified nucleic acid can include nucleotides containing e.g., arabinose, as the sugar. The modified nucleic acids can also include abasic sugars. These abasic sugars can also be further modified at one or more of the constituent sugar atoms. The modified nucleic acids can also include one or more sugars that are in the L form, e.g. L- nucleosides.

The modified nucleosides and modified nucleotides described herein, which can be incorporated into a modified nucleic acid, can include a modified base, also called a nucleobase. Examples of nucleobases include, but are not limited to, adenine (A), guanine (G), cytosine (C), and uracil (U). These nucleobases can be modified or wholly replaced to provide modified residues that can be incorporated into modified nucleic acids. The nucleobase of the nucleotide can be independently selected from a purine, a pyrimidine, a purine analog, or pyrimidine analog. In some embodiments, the nucleobase can include, for example, naturally-occurring and synthetic derivatives of a base.

In embodiments employing a dual guide RNA, each of the crRNA and the tracr RNA can contain modifications. Such modifications may be at one or both ends of the crRNA or tracr RNA. In embodiments comprising an sgRNA, one or more residues at one or both ends of the sgRNA may be chemically modified, or the entire sgRNA may be chemically modified. Certain embodiments comprise a 5′ end modification. Certain embodiments comprise a 3′ end modification. In certain embodiments, one or more or all of the nucleotides in single stranded overhang of a gRNA molecule are deoxynucleotides.

In some embodiments, the gRNAs disclosed herein comprise one of the modification patterns disclosed in WO2018/107028 A1, published Jun. 14, 2018 the contents of which are hereby incorporated by reference in their entirety.

The terms “mA,” “mC,” “mU,” or “mG” may be used to denote a nucleotide that has been modified with 2′—O-Me. The terms “fA,” “fC,” “fU,” or “fG” may be used to denote a nucleotide that has been substituted with 2′-F. A “*” may be used to depict a PS modification. The terms A*, C*, U*, or G* may be used to denote a nucleotide that is linked to the next (e.g., 3′) nucleotide with a PS bond. The terms “mA*,” “mC*,”, “mU*,” or “mG*” may be used to denote a nucleotide that has been substituted with 2′—O-Me and that is linked to the next (e.g., 3′) nucleotide with a PS bond.

In some embodiments, the gRNAs disclosed herein comprise one or more internal linkers. As used herein, “internal linker” describes a non-nucleotide segment joining two nucleotides within a guide RNA. If the gRNA contains a spacer region, the internal linker is located outside of the spacer region (e.g., in the scaffold or conserved region of the gRNA). The length of an internal linker may be dependent on, for example, the number of nucleotides replaced by the linker and the position of the linker in the gRNA. Exemplary linker-containing gRNAs are disclosed in WO 2022/261292 A1, published Dec. 15, 2022, the content of which is hereby incorporated by reference in its entirety.

gRNAs disclosed herein may comprise an internal linker. In general, any internal linker compatible with the function of the gRNA may be used. It may be desirable for the linker to have a degree of flexibility. In some embodiments, the internal linker comprises at least two, three, four, five, six, or more on-pathway single bonds. A bond is on-pathway if it is part of the shortest path of bonds between the two nucleotides whose 5′ and 3′ positions are connected to the linker.

As used herein the length of the internal linker can be defined by its bridging length. The “bridging length” of an internal linker as used herein refers to the distance or number of atoms in the shortest chain of atoms on the pathway from the first atom of the linker (bound to a 3′ substituent, such as an oxygen or phosphate, of the preceding nucleotide to the last atom of the linker (bound to a 5′ substituent, such as an oxygen or phosphate) of the following nucleotide) (e.g., from ˜ to #in the structure of Formula (I) described below). Approximate predicted bridging lengths for various linkers are provided in the table below.

Exemplary predicted linker lengths by number of atoms, number of ethylene glycol units, approximate linker length in Angstroms on the assumption that an ethylene glycol monomer is about 3.7 Angstroms, and suitable location for substitution of at least the entire loop portion of a hairpin structure are provided in the Table 28 below. Substitution of two nucleotides requires a linker length of at least about 11 Angstroms. Substitution of at least 3 nucleotides requires a linker length of at least about 16 Angstroms.

TABLE 28

| | Number of | | |
|---|---|---|---|
| Number of | Ethylene Glycol | Approximate length | Suitable location for |
| atoms | units | in Angstroms | complete loop substitution |
| 3 | 1 | 3.7 | Repeat-anti-repeat (for both loop |
| | | | and stem when no stem present) |
| 6 | 2 | 7.4 | Repeat-anti-repeat (for both loop |
| | | | and stem when no stem present) |
| 9 | 3 | 11.1 | Repeat-anti-repeat (for both loop |
| | | | and stem when no stem present), |
| | | | Nexus |
| 12 | 4 | 14.8 | Nexus |
| 15 | 5 | 18.5 | Repeat-anti-repeat, hairpin 1, |
| | | | hairpin 2 |
| 18 | 6 | 22.2 | Repeat-anti-repeat, hairpin 1, |
| | | | hairpin 2 |
| 21 | 7 | 25.9 | Repeat-anti-repeat, hairpin 1, |
| | | | hairpin 2 |
| 24 | 8 | 29.6 | Repeat-anti-repeat, hairpin 1, |
| | | | hairpin 2 |
| 27 | 9 | 33.3 | Repeat-anti-repeat, hairpin 1, |
| | | | hairpin 2 |
| 30 | 10 | 37 | Repeat-anti-repeat, hairpin 1, |
| | | | hairpin 2 |

In some embodiments, the internal linker comprises a structure of formula (I):

˜-L0-L1-L2-# (I)
wherein:
˜ indicates a bond to a 3′ substituent of the preceding nucleotide;
#indicates a bond to a 5′ substituent of the following nucleotide;
L0 is null or C₁₋₃ aliphatic;
L1 is -[E¹-(R¹)]ₘ-, where
each R¹ is independently a C₁₋₅ aliphatic group, optionally substituted with 1 or 2 E²,
each E¹ and E² are independently a hydrogen bond acceptor, or are each independently chosen from cyclic hydrocarbons, and heterocyclic hydrocarbons, and
each m is 1, 2, 3, 4, 5, 6, 7, 8, 9, or 10; and
L2 is null, C₁₋₃ aliphatic, or is a hydrogen bond acceptor.

In some embodiments, L1 comprises one or more —CH₂CH₂O—, —CH₂OCH₂—, or —OCH₂CH₂— units (“ethylene glycol subunits”). In some embodiments, the number of —CH₂CH₂O—, —CH₂OCH₂—, or —OCH₂CH₂— units is in the range of 1, 2, 3, 4, 5, 6, 7, 8, 9 or 10.

In some embodiments, m is 1, 2, 3, 4 or 5. In some embodiments, m is 1, 2, or 3. In some embodiments, m is 6, 7, 8, 9, or 10.

In some embodiments, L0 is null. In some embodiments, L0 is —CH₂— or —CH₂CH₂—.

In some embodiments, L2 is null. In some embodiments, L2 is —O—, —S—, or C₁₋₃ aliphatic. In some embodiments, L2 is —O—. In some embodiments, L2 is —S—. In some embodiments, L2 is —CH₂— or —CH₂CH₂—.

In the tables herein, L1 and L2, are optionally, C9 and C18, respectively as follows:

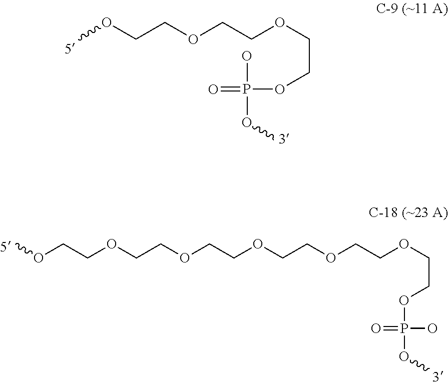

In certain embodiments, the internal linker has a bridging length of about 3-30 atoms, optionally 12-21 atoms, and the linker substitutes for at least 2 nucleotides of the gRNA. In certain embodiments, the internal linker has a bridging length of about 6-18 atoms, optionally about 6-12 atoms, and the linker substitutes for at least 2 nucleotides of the gRNA. In certain embodiments, the internal linker substitutes for 2-12 nucleotides.

In some embodiments, the guide RNA comprises a nucleic acid sequence of SEQ ID NO: 900, including modifications disclosed elsewhere herein. Table 29 shows various embodiments of the gRNA structures and species with possible number of internal linkers and positions.

TABLE 29

| gRNA | | # internal | Positions of |
|---|---|---|---|
| structures | Type | linkers | internal linkers |
| Repeat/anti- | Nme | 3 | All of repeat/anti-R; |
| R; Hp1; | | | Hp1; Hp2 |
| Hp2 | | | |
| Repeat/anti- | Nme | 2 | Any two of repeat/ |
| R; Hp1; | | | anti-R; Hp1; Hp2 |
| Hp2 | | | |
| Repeat/anti- | Nme | 1 | Any one of repeat/ |
| R; Hp1; | | | anti-R; Hp1; Hp2 |
| Hp2 | | | |

In certain embodiments, the internal linker is in a repeat-anti-repeat region of the gRNA. In certain embodiments, the internal linker substitutes for at least 4 nucleotides of the repeat-anti-repeat region of the gRNA. In certain embodiments, the internal linker substitutes for the loop in the repeat-anti-repeat region of an Nme Cas9 gRNA, corresponding to nucleotides 49-52 in SEQ ID NO: 900.

In certain embodiments, the internal linker is in a hairpin region of the gRNA. In certain embodiments, the internal linker substitutes for at least 4 nucleotides of the hairpin region of the gRNA. In certain embodiments, the internal linker substitutes for the loop in the hairpin 1 region of an Nme Cas9 gRNA, corresponding to nucleotides 87-90 in SEQ ID NO: 900. In certain embodiments, the internal linker substitutes for at least 4 nucleotides the loop in the hairpin 2 region of an Nme Cas9 gRNA, corresponding to nucleotides 122-125 in SEQ ID NO: 900. In certain embodiments, the internal linker substitutes for the loop in the hairpin 1 region of an Nme Cas9 gRNA, corresponding to nucleotides 87-90 in SEQ ID NO: 900 and for at least 4 nucleotides the loop in the hairpin 2 region of an Nme Cas9 gRNA, corresponding to nucleotides 122-125 in SEQ ID NO: 900.

TABLE 6
exemplary NmeCas9 sgRNA(SEQ ID NO: 900)

| | | | | | | | | | | | | | | | | | | | | | | | | |
|---|---|---|---|---|---|---|---|---|---|---|---|---|---|---|---|---|---|---|---|---|---|---|---|---|
| 1-24 | 25 | 26 | 27 | 28 | 29 | 30 | 31 | 32 | 33 | 34 | 35 | 36 | 37 | 38 | 39 | 40 | 41 | 42 | 43 | 44 | 45 | 46 | 47 | 48 |
| NNNNNNNNNNNNNNNNNNNNNNNN | G | U | U | G | U | A | G | C | U | C | C | C | U | U | U | C | U | C | A | U | U | U | C | G |

| | |
|---|---|
| Lower stem | Upper stem |

| Guide region | | | | | | Repeat/Anti-Repeat region | | | | | | | | | | | | | | | | | | | | | | |
|---|---|---|---|---|---|---|---|---|---|---|---|---|---|---|---|---|---|---|---|---|---|---|---|---|---|---|---|---|
| 49 | 50 | 51 | 52 | 53 | 54 | 55 | 56 | 57 | 58 | 59 | 60 | 61 | 62 | 63 | 64 | 65 | 66 | 67 | 68 | 69 | 70 | 71 | 72 | 73 | 74 | 75 | 76 | 77 |
| G | A | A | A | C | G | A | A | A | U | G | A | G | A | A | C | C | G | U | U | G | C | U | A | C | A | A | U | A |

| | | |
|---|---|---|
| Loop | Upper stem | Lower stem |

Repeat/Anti-Repeat region

| | | | | | | | | | | | | | | | | | | | | | | | | | | | | | | |
|---|---|---|---|---|---|---|---|---|---|---|---|---|---|---|---|---|---|---|---|---|---|---|---|---|---|---|---|---|---|---|
| 78 | 79 | 80 | 81 | 82 | 83 | 84 | 85 | 86 | 87 | 88 | 89 | 90 | 91 | 92 | 93 | 94 | 95 | 96 | 97 | 98 | 99 | 100 | 101 | 102 | 103 | 104 | 105 | 106 | 107 | 108 |
| A | G | G | C | C | G | U | C | U | G | A | A | A | A | G | A | U | G | U | G | C | C | G | C | A | A | C | G | C | U | C |

| | | | | |
|---|---|---|---|---|
| Stem | Loop | Stem (96: unpaired) | Lower stem | Bulge |

| | |
|---|---|
| Hairpin 1 | Hairpin 2 |

| | | | | | | | | | | | | | | | | | | | | | | | | | |
|---|---|---|---|---|---|---|---|---|---|---|---|---|---|---|---|---|---|---|---|---|---|---|---|---|---|
| 109 | 110 | 111 | 112 | 113 | 114 | 115 | 116 | 117 | 118 | 119 | 120 | 121 | 122 | 123 | 124 | 125 | 126 | 127 | 128 | 129 | 130 | 131 | 132 | 133 | 134 |
| U | G | C | C | C | C | U | U | A | A | A | G | C | U | U | C | U | G | C | U | U | U | A | A | G | G |

| | | |
|---|---|---|
| Upper Stem | Loop | Upper Stem |

Hairpin 2

| | | | | | | | | | | |
|---|---|---|---|---|---|---|---|---|---|---|
| 135 | 136 | 137 | 138 | 139 | 140 | 141 | 142 | 143 | 144 | 145 |
| G | G | C | A | U | C | G | U | U | U | A |

| | | |
|---|---|---|
| Upper Stem | Bulge | Lower Stem |

| | |
|---|---|
| Hairpin 2 | Tail |

### B. Ribonucleoprotein Complex

In some embodiments, the disclosure provides compositions comprising one or more gRNAs comprising one or more guide sequences from Tables 1-5 and an RNA-guided DNA binding agent, e.g., a nuclease, such as a Cas nuclease, such as Cas9. In some embodiments, the RNA-guided DNA-binding agent has cleavase activity, which can also be referred to as double-strand endonuclease activity. In some embodiments, the RNA-guided DNA-binding agent comprises a Cas nuclease. Examples of Cas9 nucleases include those of the type II CRISPR systems of N. meningitidis and other prokaryotes known in the art, and modified (e.g., engineered or mutant) versions thereof. In some embodiments, the RNA-guided DNA-binding agent comprises a Cas nickase. In some embodiments, the RNA-guided nickase is modified or derived from a Cas protein, such as a Class 2 Cas nuclease (which may be, e.g., a Cas nuclease of Type II). Class 2 Cas nuclease include, for example, Cas9 proteins and modifications thereof.

In some embodiments, the Cas nuclease is the Cas9 nuclease from Neisseria meningitidis.

In some embodiments, the Cas nickase is a nickase form of the Cas9 nuclease from Neisseria meningitidis. See e.g., WO/2020081568, describing an Nme2Cas9 D16A nickase fusion protein.

In some embodiments, the gRNA together with an RNA-guided DNA binding agent is called a ribonucleoprotein complex (RNP). In some embodiments, the RNA-guided DNA binding agent is a Cas nuclease. In some embodiments, the gRNA together with a Cas nuclease is called a Cas RNP. In some embodiments, the RNP comprises Type-II components. In some embodiments, the Cas nuclease is the Cas9 protein from the Type-II CRISPR/Cas system. In some embodiment, the gRNA together with Cas9 is called a Cas9 RNP.

Wild type Cas9 has two nuclease domains: RuvC and HNH. The RuvC domain cleaves the non-target DNA strand, and the HNH domain cleaves the target strand of DNA. In some embodiments, the Cas9 protein comprises more than one RuvC domain or more than one HNH domain. In some embodiments, the Cas9 protein is a wild type Cas9. In each of the composition, use, and method embodiments, the Cas induces a double strand break in target DNA.

In some embodiments, chimeric Cas nucleases are used, where one domain or region of the protein is replaced by a portion of a different protein. In some embodiments, a Cas nuclease domain may be replaced with a domain from a different nuclease such as FokI. In some embodiments, a Cas nuclease may be a modified nuclease.

In some embodiments, the RNA-guided DNA-binding agent has single-strand nickase activity, i.e., can cut one DNA strand to produce a single-strand break, also known as a “nick.” In some embodiments, the RNA-guided DNA-binding agent comprises a Cas nickase. A nickase is an enzyme that creates a nick in dsDNA, i.e., cuts one strand but not the other of the DNA double helix. In some embodiments, a Cas nickase is a version of a Cas nuclease (e.g., a Cas nuclease discussed above) in which an endonucleolytic active site is inactivated, e.g., by one or more alterations (e.g., point mutations) in a catalytic domain. See e.g., U.S. Pat. No. 8,889,356 for discussion of Cas nickases and exemplary catalytic domain alterations. In some embodiments, a Cas nickase such as a Cas9 nickase has an inactivated RuvC or HNH domain.

In some embodiments, the RNA-guided DNA-binding agent is modified to contain only one functional nuclease domain. For example, the agent protein may be modified such that one of the nuclease domains is mutated or fully or partially deleted to reduce its nucleic acid cleavage activity. In some embodiments, a nickase is used having a RuvC domain with reduced activity. In some embodiments, a nickase is used having an inactive RuvC domain. In some embodiments, a nickase is used having an HNH domain with reduced activity. In some embodiments, a nickase is used having an inactive HNH domain.

In some embodiments, a conserved amino acid within a Cas protein nuclease domain is substituted to reduce or alter nuclease activity. In some embodiments, a Cas nuclease may comprise an amino acid substitution in the RuvC or RuvC-like nuclease domain. Exemplary amino acid substitutions in the HNH or HNH-like nuclease domain or RuvC or RuvC-like domains for N. meningitidis include Nme2Cas9D16A (HNH nickase) and Nme2Cas9H588A (RuvC nickase).

In some embodiments, an mRNA encoding a nickase is provided in combination with a pair of guide RNAs that are complementary to the sense and antisense strands of the target sequence, respectively. In this embodiment, the guide RNAs direct the nickase to a target sequence and introduce a DSB by generating a nick on opposite strands of the target sequence (i.e., double nicking). In some embodiments, use of double nicking may improve specificity and reduce off-target effects. In some embodiments, a nickase is used together with two separate guide RNAs targeting opposite strands of DNA to produce a double nick in the target DNA. In some embodiments, a nickase is used together with two separate guide RNAs that are selected to be in close proximity to produce a double nick in the target DNA.

In some embodiments, the RNA-guided DNA-binding agent lacks cleavase and nickase activity. In some embodiments, the RNA-guided DNA-binding agent comprises a dCas DNA-binding polypeptide. A dCas polypeptide has DNA-binding activity while essentially lacking catalytic (cleavase/nickase) activity. In some embodiments, the dCas polypeptide is a dCas9 polypeptide. In some embodiments, the RNA-guided DNA-binding agent lacking cleavase and nickase activity or the dCas DNA-binding polypeptide is a version of a Cas nuclease (e.g., a Cas nuclease discussed above) in which its endonucleolytic active sites are inactivated, e.g., by one or more alterations (e.g., point mutations) in its catalytic domains. See, e.g., US 2014/0186958 A1; US 2015/0166980 A1.

In some embodiments, the RNA-guided DNA binding agent comprises one or more heterologous functional domains (e.g., is or comprises a fusion polypeptide).

In some embodiments, the RNA-guided DNA binding agent comprises a APOBEC3 deaminase. In some embodiments, a APOBEC3 deaminase is a APOBEC3A (A3A). In some embodiments, the A3A is a human A3A. In some embodiments, the A3A is a wild-type A3A.

In some embodiments, the RNA-guided DNA binding agent comprises a deaminase and an RNA-guided nickase. In some embodiments, the mRNA further comprises a linker to link the sequencing encoding A3A to the sequence sequencing encoding RNA-guided nickase. In some embodiments, the linker is an organic molecule, group, polymer, or chemical moiety. In some embodiments, the linker is a peptide linker. In some embodiments, the peptide linker is any stretch of amino acids having at least 1, at least 2, at least 3, at least 4, at least 5, at least 6, at least 7, at least 8, at least 9, at least 10, at least 15, at least 20, at least 25, at least 30, at least 40, at least 50, or more amino acids. In some embodiments, the peptide linker is the 16 residue “XTEN” linker, or a variant thereof (See, e.g., the Examples; and Schellenberger et al. A recombinant polypeptide extends the in vivo half-life of peptides and proteins in a tunable manner. Nat. Biotechnol. 27, 1186-1190 (2009)).

In some embodiments, the peptide linker is the 16 residue “XTEN” linker, or a variant thereof (See, e.g., the Examples; and Schellenberger et al. A recombinant polypeptide extends the in vivo half-life of peptides and proteins in a tunable manner. Nat. Biotechnol. 27, 1186-1190 (2009)). In some embodiments, the XTEN linker comprises the sequence SGSETPGTSESATPES (SEQ ID NO: 930), SGSETPGTSESA (SEQ ID NO: 931), or SGSETPGTSESATPEGGSGGS (SEQ ID NO: 932).

In some embodiments, the peptide linker comprises a (GGGGS)ₙ(SEQ ID NO: 998), a (G)ₙ, an (EAAAK)ₙ(SEQ ID NO: 935), a (GGS)ₙ, an SGSETPGTSESATPES (SEQ ID NO: 930) motif (see, e.g., Guilinger J P, Thompson D B, Liu D R. Fusion of catalytically inactive Cas9 to FokI nuclease improves the specificity of genome modification. Nat. Biotechnol. 2014; 32(6): 577-82; the entire contents are incorporated herein by reference), or an (XP)ₙ motif, or a combination of any of these, wherein n is independently an integer between 1 and 30 (SEQ ID NO: 3012). See, WO2015089406, e.g., paragraph [0012], the entire content of which is incorporated herein by reference.

In some embodiments, the peptide linker comprises one or more sequences selected from SEQ ID NOs: 930-994.

In some embodiments, the heterologous functional domain may facilitate transport of the RNA-guided DNA-binding agent into the nucleus of a cell. For example, the heterologous functional domain may be a nuclear localization signal (NLS). In some embodiments, the RNA-guided DNA-binding agent may be fused with 1-5 NLS(s). In some embodiments, the RNA-guided DNA-binding agent may be fused with 2, 3, or 4 NLS(s). In some embodiments, the RNA-guided DNA-binding agent may be fused with two NLS(s). In some embodiments, the RNA-guided DNA-binding agent may be fused with one NLS. Where one NLS is used, the NLS may be linked at the N-terminus or the C-terminus of the RNA-guided DNA-binding agent sequence. In some embodiments, the NLS is not linked to the C-terminus. It may also be inserted within the RNA-guided DNA binding agent sequence. In certain circumstances, at least the two NLSs are the same (e.g., two SV40 NLSs). In certain embodiments, at least two different NLSs are present the RNA-guided DNA binding agent. In some embodiments, the RNA-guided DNA-binding agent is fused to two SV40 NLS sequences linked at the carboxy terminus. In some embodiments, the RNA-guided DNA-binding agent may be fused with two NLSs, one linked at the N-terminus and one at the C-terminus. In some embodiments, the RNA-guided DNA-binding agent may be fused with 3 NLSs. In some embodiments, the RNA-guided DNA-binding agent may be fused with no NLS. In some embodiments, the NLS may be a monopartite sequence, such as, e.g., the SV40 NLS, PKKKRKV (SEQ ID NO: 3013) or PKKKRRV (SEQ ID NO: 923). In some embodiments, the NLS may be a bipartite sequence, such as the NLS of nucleoplasmin, KRPAATKKAGQAKKKK (SEQ ID NO: 924). In a specific embodiment, a single PKKKRKV (SEQ ID NO: 3013) NLS may be linked at the C-terminus of the RNA-guided DNA-binding agent. One or more linkers are optionally included at the fusion site. In some embodiments, the NLS comprises one or more sequences selected from SEQ ID NOs: 912-924.

In some embodiments, the RNA-guided DNA binding agent comprises an editor. An exemplary editor is BC22n which includes a H. sapiens APOBEC3A fused to Nme D16A Cas9 nickase by an XTEN linker, and mRNA encoding BC22n. An mRNA encoding NmeCas9 BC22n is provided (SEQ ID NO: 822).

In some embodiments, the heterologous functional domain may be capable of modifying the intracellular half-life of the RNA-guided DNA binding agent. In some embodiments, the half-life of the RNA-guided DNA binding agent may be increased. In some embodiments, the half-life of the RNA-guided DNA-binding agent may be reduced. In some embodiments, the heterologous functional domain may be capable of increasing the stability of the RNA-guided DNA-binding agent. In some embodiments, the heterologous functional domain may be capable of reducing the stability of the RNA-guided DNA-binding agent. In some embodiments, the heterologous functional domain may act as a signal peptide for protein degradation. In some embodiments, the protein degradation may be mediated by proteolytic enzymes, such as, for example, proteasomes, lysosomal proteases, or calpain proteases. In some embodiments, the heterologous functional domain may comprise a PEST sequence. In some embodiments, the RNA-guided DNA-binding agent may be modified by addition of ubiquitin or a polyubiquitin chain. In some embodiments, the ubiquitin may be a ubiquitin-like protein (UBL). Non-limiting examples of ubiquitin-like proteins include small ubiquitin-like modifier (SUMO), ubiquitin cross-reactive protein (UCRP, also known as interferon-stimulated gene-15 (ISG15)), ubiquitin-related modifier-1 (URM1), neuronal-precursor-cell-expressed developmentally downregulated protein-8 (NEDD8, also called Rubl in S. cerevisiae), human leukocyte antigen F-associated (FAT10), autophagy-8 (ATG8) and -12 (ATG12), Fau ubiquitin-like protein (FUB1), membrane-anchored UBL (MUB), ubiquitin fold-modifier-1 (UFM1), and ubiquitin-like protein-5 (UBL5).

In some embodiments, the heterologous functional domain may be a marker domain. Non-limiting examples of marker domains include fluorescent proteins, purification tags, epitope tags, and reporter gene sequences. In some embodiments, the marker domain may be a fluorescent protein. Non-limiting examples of suitable fluorescent proteins include green fluorescent proteins (e.g., GFP, GFP-2, tagGFP, turboGFP, sfGFP, EGFP, Emerald, Azami Green, Monomeric Azami Green, CopGFP, AceGFP, ZsGreen1), yellow fluorescent proteins (e.g., YFP, EYFP, Citrine, Venus, YPet, PhiYFP, ZsYellow1), blue fluorescent proteins (e.g., EBFP, EBFP2, Azurite, mKalamal, GFPuv, Sapphire, T-sapphire), cyan fluorescent proteins (e.g., ECFP, Cerulean, CyPet, AmCyani, Midoriishi-Cyan), red fluorescent proteins (e.g., mKate, mKate2, mPlum, DsRed monomer, mCherry, mRFP1, DsRed-Express, DsRed2, DsRed-Monomer, HcRed-Tandem, HcRed1, AsRed2, eqFP611, mRasberry, mStrawberry, Jred), and orange fluorescent proteins (mOrange, mKO, Kusabira-Orange, Monomeric Kusabira-Orange, mTangerine, tdTomato) or any other suitable fluorescent protein. In other embodiments, the marker domain may be a purification tag or an epitope tag. Non-limiting exemplary tags include glutathione-S-transferase (GST), chitin binding protein (CBP), maltose binding protein (MBP), thioredoxin (TRX), poly(NANP), tandem affinity purification (TAP) tag, myc, AcV5, AU1, AU5, E, ECS, E2, FLAG, HA, nus, Softag 1, Softag 3, Strep, SBP, Glu-Glu, HSV, KT3, S, S1, T7, V5, VSV-G, 6×His (SEQ ID NO: 3014), 8×His (SEQ ID NO: 3015), biotin carboxyl carrier protein (BCCP), poly-His, and calmodulin. Non-limiting exemplary reporter genes include glutathione-S-transferase (GST), horseradish peroxidase (HRP), chloramphenicol acetyltransferase (CAT), beta-galactosidase, beta-glucuronidase, luciferase, or fluorescent proteins.

In additional embodiments, the heterologous functional domain may target the RNA-guided DNA-binding agent to a specific organelle, cell type, tissue, or organ. In some embodiments, the heterologous functional domain may target the RNA-guided DNA-binding agent to mitochondria.

In further embodiments, the heterologous functional domain may be an effector domain such as an editor domain. When the RNA-guided DNA-binding agent is directed to its target sequence, e.g., when a Cas nuclease is directed to a target sequence by a gRNA, the effector such as an editor domain may modify or affect the target sequence. In some embodiments, the effector such as an editor domain may be chosen from a nucleic acid binding domain, a nuclease domain (e.g., a non-Cas nuclease domain), an epigenetic modification domain, a transcriptional activation domain, or a transcriptional repressor domain. In some embodiments, the heterologous functional domain is a nuclease, such as a FokI nuclease. See, e.g., U.S. Pat. No. 9,023,649. In some embodiments, the heterologous functional domain is a transcriptional activator or repressor. See, e.g., Qi et al., “Repurposing CRISPR as an RNA-guided platform for sequence-specific control of gene expression,” Cell 152:1173-83 (2013); Perez-Pinera et al., “RNA-guided gene activation by CRISPR-Cas9-based transcription factors,” Nat. Methods 10:973-6 (2013); Mali et al., “CAS9 transcriptional activators for target specificity screening and paired nickases for cooperative genome engineering,” Nat. Biotechnol. 31:833-8 (2013); Gilbert et al., “CRISPR-mediated modular RNA-guided regulation of transcription in eukaryotes,” Cell 154:442-51 (2013). As such, the RNA-guided DNA-binding agent essentially becomes a transcription factor that can be directed to bind a desired target sequence using a guide RNA.

### C. Determination of Efficacy of Guide RNAs

In some embodiments, the efficacy of a guide RNA is determined when delivered or expressed together with other components (e.g., an RNA-guided DNA binding agent) forming an RNP. In some embodiments, the guide RNA is expressed together with an RNA-guided DNA binding agent, such as a Cas protein, e.g., Cas9. In some embodiments, the guide RNA is delivered to or expressed in a cell line that already stably expresses an RNA-guided DNA nuclease, such as a Cas nuclease or nickase, e.g., Cas9 nuclease or nickase. In some embodiments the guide RNA is delivered to a cell as part of a RNP. In some embodiments, the guide RNA is delivered to a cell along with a mRNA encoding an RNA-guided DNA nuclease, such as a Cas nuclease or nickase, e.g., Cas9 nuclease or nickase.

As described herein, use of an RNA-guided DNA nuclease and a guide RNA disclosed herein can lead to DSBs, SSBs, or site-specific binding that results in nucleic acid modification in the DNA or pre-mRNA which can produce errors in the form of insertion/deletion (indel) mutations upon repair by cellular machinery. Many mutations due to indels alter the reading frame, introduce premature stop codons, or induce exon skipping and, therefore, produce a non-functional protein.

In some embodiments, the efficacy of particular guide RNAs is determined based on in vitro models. In some embodiments, the in vitro model is T cell line. In some embodiments, the in vitro model is HEK293 T cells. In some embodiments, the in vitro model is HEK293 cells stably expressing Cas9 (HEK293_Cas9). In some embodiments, the in vitro model is a lymphoblastoid cell line. In some embodiments, the in vitro model is primary human T cells. In some embodiments, the in vitro model is primary human B cells. In some embodiments, the in vitro model is primary human peripheral blood lymphocytes. In some embodiments, the in vitro model is primary human peripheral blood mononuclear cells.

In some embodiments, the number of off-target sites at which a deletion or insertion occurs in an in vitro model is determined, e.g., by analyzing genomic DNA from the cells transfected in vitro with Cas9 mRNA and the guide RNA. In some embodiments, such a determination comprises analyzing genomic DNA from cells transfected in vitro with Cas9 mRNA, the guide RNA, and a donor oligonucleotide. Exemplary procedures for such determinations are provided in the working examples below.

In some embodiments, the efficacy of particular gRNAs is determined across multiple in vitro cell models for a guide RNA selection process. In some embodiments, a cell line comparison of data with selected guide RNAs is performed. In some embodiments, cross screening in multiple cell models is performed.

In some embodiments, the efficacy of particular guide RNAs is determined based on in vivo models. In some embodiments, the in vivo model is a rodent model. In some embodiments, the rodent model is a mouse which expresses the target gene. In some embodiments, the rodent model is a mouse which expresses an HLA-A gene. In some embodiments, the rodent model is a mouse which expresses a human HLA-A gene. In some embodiments, the rodent model is a mouse which expresses an HLA-B gene. In some embodiments, the rodent model is a mouse which expresses a human HLA-B gene. In some embodiments, the rodent model is a mouse which expresses a TRAC gene. In some embodiments, the rodent model is a mouse which expresses a human TRAC gene. In some embodiments, the rodent model is a mouse which expresses a TRBC1 gene. In some embodiments, the rodent model is a mouse which expresses a human TRBC1 gene. In some embodiments, the rodent model is a mouse which expresses a human TRBC2 gene. In some embodiments, the rodent model is a mouse which expresses a TRBC2 gene. In some embodiments, the rodent model is a mouse which expresses a CIITA gene. In some embodiments, the rodent model is a mouse which expresses a human CIITA gene. In some embodiments, the in vivo model is a non-human primate, for example cynomolgus monkey.

In some embodiments, the efficacy of a guide RNA is evaluated by on target cleavage efficiency. In some embodiments, the efficacy of a guide RNA is measured by percent editing at the target location, e.g., HLA-A, TRAC, TRBC, CIITA, or AAVS1. In some embodiments, deep sequencing may be utilized to identify the presence of modifications (e.g., insertions, deletions) introduced by genomic editing. Indel percentage can be calculated from next generation sequencing “NGS.”

In some embodiments, the efficacy of a guide RNA is measured by the number or frequency of indels at off-target sequences within the genome of the target cell type. In some embodiments, efficacious guide RNAs are provided which produce indels at off target sites at very low frequencies (e.g., <5%) in a cell population or relative to the frequency of indel creation at the target site. Thus, the disclosure provides for guide RNAs which do not exhibit off-target indel formation in the target cell type (e.g., T cells or B cells), or which produce a frequency of off-target indel formation of <5% in a cell population or relative to the frequency of indel creation at the target site. In some embodiments, the disclosure provides guide RNAs which do not exhibit any off target indel formation in the target cell type (e.g., T cells or B cells). In some embodiments, guide RNAs are provided which produce indels at less than 5 off-target sites, e.g., as evaluated by one or more methods described herein. In some embodiments, guide RNAs are provided which produce indels at less than or equal to 4, 3, 2, or 1 off-target site(s) e.g., as evaluated by one or more methods described herein. In some embodiments, the off-target site(s) does not occur in a protein coding region in the target cell (e.g., T cells or B cells) genome.

In some embodiments, linear amplification is used to detect genomic editing events, such as the formation of insertion/deletion (“indel”) mutations, translocations, and homology directed repair (HDR) events in target DNA. For example, linear amplification with a unique sequence-tagged primer and isolating the tagged amplification products (herein after referred to as “UnIT,” or “Unique Identifier Tagmentation” method) may be used.

In some embodiments, the efficacy of a guide RNA is measured by the number of chromosomal rearrangements within the target cell type. Kromatid dGH assay may used to detect chromosomal rearrangements, including e.g., translocations, reciprocal translocations, translocations to off-target chromosomes, deletions (i.e., chromosomal rearrangements where fragments were lost during the cell replication cycle due to the editing event). In some embodiments, the target cell type has less than 10, less than 8, less than 5, less than 4, less than 3, less than 2, or less than 1 chromosomal rearrangement. In some embodiments, the target cell type has no chromosomal rearrangements.

D. Delivery of gRNA Compositions

Lipid nanoparticles (LNPs) are a well-known means for delivery of nucleotide and protein cargo and may be used for delivery of the guide RNAs, compositions, or pharmaceutical formulations disclosed herein. In some embodiments, the LNPs deliver nucleic acid, protein, or nucleic acid together with protein.

In some embodiments, the invention comprises a method for delivering any one of the gRNAs disclosed herein to a subject, wherein the gRNA is formulated as an LNP. In some embodiments, the LNP comprises the gRNA and a Cas9 or an mRNA encoding Cas9.

In some embodiments, the invention comprises a composition comprising any one of the gRNAs disclosed and an LNP. In some embodiments, the composition further comprises a Cas9 or an mRNA encoding Cas9.

In some embodiments, the LNPs comprise cationic lipids. In some embodiments, the LNPs comprise (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate) (Lipid A) or another ionizable lipid. See, e.g., lipids of WO/2017/173054 and references described therein. In some embodiments, the LNPs comprise molar ratios of a cationic lipid amine to RNA phosphate (N:P) of about 4.5, 5.0, 5.5, 6.0, or 6.5. In some embodiments, the term cationic and ionizable in the context of LNP lipids is interchangeable, e.g., wherein ionizable lipids are cationic depending on the pH.

In some embodiments, the LNP comprises a lipid component, and the lipid component comprises: about 35 mol % Lipid A; about 15 mol % neutral lipid (e.g., distearoylphosphatidylcholine (DSPC)); about 47.5 mol % helper lipid (e.g., cholesterol); and about 2.5 mol % stealth lipid (e.g., 1,2-dimyristoyl-rac-glycero-3-methylpolyoxyethylene glycol 2000 (PEG2k-DMG)), and wherein the N/P ratio of the LNP composition is about 3-7.

In some embodiments, the gRNAs disclosed herein are formulated as LNPs for use in preparing a medicament for treating a disease or disorder.

Electroporation is a well-known means for delivery of cargo, and any electroporation methodology may be used for delivery of any one of the gRNAs disclosed herein. In some embodiments, electroporation may be used to deliver any one of the gRNAs disclosed herein and Cas9 or an mRNA encoding Cas9.

In some embodiments, the invention comprises a method for delivering any one of the gRNAs disclosed herein to an ex vivo cell, wherein the gRNA is formulated as an LNP or not formulated as an LNP. In some embodiments, the LNP comprises the gRNA and a Cas9 or an mRNA encoding Cas9.

In some embodiments, the guide RNA compositions described herein, alone or encoded on one or more vectors, are formulated in or administered via a lipid nanoparticle; see e.g., WO/2017/173054 and WO 2019/067992, the contents of which are hereby incorporated by reference in their entirety.

In certain embodiments, the invention comprises DNA or RNA vectors encoding any of the guide RNAs comprising any one or more of the guide sequences described herein. In some embodiments, in addition to guide RNA sequences, the vectors further comprise nucleic acids that do not encode guide RNAs. Nucleic acids that do not encode guide RNA include, but are not limited to, promoters, enhancers, regulatory sequences, and nucleic acids encoding an RNA-guided DNA nuclease, which can be a nuclease such as Cas9. In some embodiments, the vector comprises one or more nucleotide sequence(s) encoding a crRNA, a trRNA, or a crRNA and trRNA. In some embodiments, the vector comprises one or more nucleotide sequence(s) encoding a sgRNA and an mRNA encoding an RNA-guided DNA nuclease, which can be a Cas nuclease, such as Cas9. In some embodiments, the vector comprises one or more nucleotide sequence(s) encoding a crRNA, a trRNA, and an mRNA encoding an RNA-guided DNA nuclease, which can be a Cas protein, such as, Cas9. In one embodiment, the Cas9 is from N. meningitidis (i.e., Nine Cas9). In some embodiments, the nucleotide sequence encoding the crRNA, trRNA, or crRNA and trRNA (which may be a sgRNA) comprises or consists of a guide sequence flanked by all or a portion of a repeat sequence from a naturally-occurring CRISPR/Cas system. The nucleic acid comprising or consisting of the crRNA, trRNA, or crRNA and trRNA may further comprise a vector sequence wherein the vector sequence comprises or consists of nucleic acids that are not naturally found together with the crRNA, trRNA, or crRNA and trRNA.

### IV. Therapeutic Methods and Uses

Any of the engineered cells and compositions described herein can be used in a method of treating a variety of diseases and disorders, as described herein. In some embodiments, the genetically modified cell (engineered cell) or population of genetically modified cells (engineered cells) and compositions may be used in methods of treating a variety of diseases and disorders. In some embodiments, a method of treating any one of the diseases or disorders described herein is encompassed, comprising administering any one or more composition described herein.

In some embodiments, the methods and compositions described herein may be used to treat diseases or disorders in need of delivery of a therapeutic agent. In some embodiments, the invention provides a method of providing an immunotherapy in a subject, the method including administering to the subject an effective amount of an engineered cell (or population of engineered cells) as described herein, for example, a cell of any of the aforementioned cell aspects and embodiments.

In some embodiments, the methods comprise administering to a subject a composition comprising an engineered cell described herein as an adoptive cell transfer therapy. In some embodiments, the engineered cell is an allogeneic cell.

In some embodiments of the methods, the method includes administering a lymphodepleting agent or immunosuppressant prior to administering to the subject an effective amount of the engineered cell (or engineered cells) as described herein, for example, a cell of any of the aforementioned cell aspects and embodiments. In another aspect, the invention provides a method of preparing engineered cells (e.g., a population of engineered cells).

Immunotherapy is the treatment of disease by activating or suppressing the immune system. Immunotherapies designed to elicit or amplify an immune response are classified as activation immunotherapies. Cell-based immunotherapies have been demonstrated to be effective in the treatment of some cancers. Immune effector cells such as lymphocytes, macrophages, dendritic cells, natural killer cells, cytotoxic T lymphocytes (CTLs), T helper cells, B cells, or their progenitors such as hematopoietic stem cells (HSC) or induced pluripotent stem cells (iPSC) can be programmed to act in response to abnormal antigens expressed on the surface of tumor cells. Thus, cancer immunotherapy allows components of the immune system to destroy tumors or other cancerous cells. Cell-based immunotherapies have also been demonstrated to be effective in the treatment of autoimmune diseases or transplant rejection. Immune effector cells such as regulatory T cells (Tregs) or mesenchymal stem cells can be programmed to act in response to autoantigens or transplant antigens expressed on the surface of normal tissues.

In some embodiments, the invention provides a method of preparing engineered cells (e.g., a population of engineered cells). The population of engineered cells may be used for immunotherapy.

In some embodiments, the invention provides a method of treating a subject in need thereof that includes administering engineered cells prepared by a method of preparing cells described herein, for example, a method of any of the aforementioned aspects and embodiments of methods of preparing cells.

In some embodiments, the engineered cells can be used to treat cancer, infectious diseases, inflammatory diseases, autoimmune diseases, cardiovascular diseases, neurological diseases, ophthalmologic diseases, renal diseases, liver diseases, musculoskeletal diseases, red blood cell diseases, or transplant rejections. In some embodiments, the engineered cells can be used in cell transplant, e.g., to the heart, liver, lung, kidney, pancreas, skin, or brain. (See e.g., Deuse et al., Nature Biotechnology 37:252-258 (2019).)

In some embodiments, the engineered cells can be used as a cell therapy comprising an allogeneic stem cell therapy. In some embodiments, the cell therapy comprises induced pluripotent stem cells (iPSCs). iPSCs may be induced to differentiate into other cell types including e.g., beta islet cells, neurons, and blood cells. In some embodiments, the cell therapy comprises hematopoietic stem cells. In some embodiments, the stem cells comprise mesenchymal stem cells that can develop into bone, cartilage, muscle, and fat cells. In some embodiments, the stem cells comprise ocular stem cells. In some embodiments, the allogeneic stem cell transplant comprises allogeneic bone marrow transplant. In some embodiments, the stem cells comprise pluripotent stem cells (PSCs). In some embodiments, the stem cells comprise induced embryonic stem cells (ESCs).

In some embodiments, the methods provide for administering the engineered cells to a subject, wherein the administration is an injection. In some embodiments, the methods provide for administering the engineered cells to a subject, wherein the administration is an intravascular injection or infusion. In some embodiments, the methods provide for administering the engineered cells to a subject, wherein the administration is a single dose.

In some embodiments, the methods provide for reducing a sign or symptom associated of a subject's disease treated with a composition disclosed herein. In some embodiments, the subject has a response to treatment with a composition disclosed herein that lasts more than one week. In some embodiments, the subject has a response to treatment with a composition disclosed herein that lasts more than two weeks. In some embodiments, the subject has a response to treatment with a composition disclosed herein that lasts more than three weeks. In some embodiments, the subject has a response to treatment with a composition disclosed herein that lasts more than one month.

In some embodiments, the methods provide for administering the engineered cells to a subject, and wherein the subject has a response to the administered cell that comprises a reduction in a sign or symptom associated with the disease treated by the cell therapy. In some embodiments, the subject has a response that lasts more than one week. In some embodiments, the subject has a response that lasts more than one month. In some embodiments, the subject has a response that lasts for at least 1-6 weeks.

TABLE7
V. Additional Sequences

| Description | SEQ ID NO | Sequence |
|---|---|---|
| HLA-A | 801 | mG*mG*mC*CACGGAGCGAGACAUCUGUUUUAGAmGmCmUmAmGmAmAmAmUmAmGmCAAGUUAAAAUAAGGCUAGUCCGUUAUCAmAmCmUm |
| S. pyogenes | | UmGmAmAmAmAmAmGmUmGmGmCmAmCmCmGmAmGmUmCmGmGmUmGmCmU*mU*mU*mU |
| guide | | |
| G000529 | | |
| HLA-A | 802 | mA*mC*mA*GCGACGCCGCGAGCCAGGUUUUAGAmGmCmUmAmGmAmAmAmUmAmGmCAAGUUAAAAUAAGGCUAGUCCGUUAUCAmAmCmUm |
| control guide | | UmGmAmAmAmAmAmGmUmGmGmCmAmCmCmGmAmGmUmCmGmGmUmGmCmU*mU*mU*mU |
| G018995 | | |
| TRAC | 803 | mU*mU*mC*AAAACCUGUCAGUGAUUGUUUUAGAmGmCmUmAmGmAmAmAmUmAmGmCAAGUUAAAAUAAGGUAGUCCGUUAUCACGAAAGGG |
| control guide | | CACCGAGUCGGmU*mG*mC*mU |
| (G027891) | | |
| TRAC | 804, 3206, and 3207 | MG*MC*MC*MGMUGUMAMCCMAGMCUGAGMAGACMUCUMGUUGMUMAMGMCUCCCMU(L1)MCMCGUUMGMCUAMCAAU*AAGMGMCCMGMUM |
| control guide | | C(L1)MGMAMUGUGCMCGMCAAMCGCUCUMGMCC(L1)GGCAUCGMU*MU |
| (G023516) | | |
| TRAC | 805 | MC*MC*MC*MAMCAGMAMUAMUCMCAGAAMCCCUMGACMGUUGMUMAMGMCUCCCMUMGMAMAMAMCMCGUUMGMCUAMCAAU*AAGMGMCCM |
| control guide | | GMUMCMGMAMAMAMGMAMUGUGCMCGMCAAMCGCUCUMGMCCMUMUMCMUGGCAUCG*MU*MU |
| (G021470) | | |
| TRAC | 806 | MU*MU*MG*MUMCCCMAMCAMGAMUAUCCMAGAAMCCCMGUUGMUMAMGMCUCCCMUMGMAMAMAMCMCGUUMGMCUAMCAAU*AAGMGMCCM |
| control guide | | GMUMCMGMAMAMAMGMAMUGUGCMCGMCAAMCGCUCUMGMCCMUMUMCMUGGCAUCG*MU*MU |
| (G021471) | | |
| TRAC | 807 | mC*mU*mU*mGmUCCmCmACmAGmAUAUCmCAGAmACCmGUUGmUmAmGmCUCCCmUmGmAmAmAmCmCGUUmGmCUAmCAAU*AAGmGmCCm |
| control guide | | GmUmCmGmAmAmAmGmAmUGUGCmCGmCAAmCGCUCUmGmCCmUmUmCmUGGCAUCG*mU*mU |
| (G021472) | | |
| TRAC | 808 | mA*mU*mC*mCmUCUmUmGUmCCmCACAGmAUAUmCCAmGUUGmUmAmGmCUCCCmUmGmAmAmAmCmCGUUmGmCUAmCAAU*AAGmGmCCm |
| control guide | | GmUmCmGmAmAmAmGmAmUGUGCmCGmCAAmCGCUCUmGmCCmUmUmCmUGGCAUCG*mU*mU |
| (G021473) | | |
| TRAC | 809 | mG*mA*mU*mCmCUCmUmUGmUCmCCACAmGAUAmUCCmGUUGmUmAmGmCUCCCmUmGmAmAmAmCmCGUUmGmCUAmCAAU*AAGmGmCCm |
| control guide | | GmUmCmGmAmAmAmGmAmUGUGCmCGmCAAmCGCUCUmGmCCmUmUmCmUGGCAUCG*mU*mU |
| (G021474) | | |
| TRAC | 810 | mA*mA*mC*mCmCUGmAmUCmCUmCUUGUmCCCAmCAGmGUUGmUmAmGmCUCCCmUmGmAmAmAmCmCGUUmGmCUAmCAAU*AAGmGmCCm |
| control guide | | GmUmCmGmAmAmAmGmAmUGUGCmCGmCAAmCGCUCUmGmCCmUmUmCmUGGCAUCG*mU*mU |
| (G021475) | | |
| TRAC | 811 | mG*mC*mC*mGmUGUmAmCCmAGmCUGAGmAGACmUCUmGUUGmUmAmGmCUCCCmUmGmAmAmAmCmCGUUmGmCUAmCAAU*AAGmGmCCm |
| control guide | | GmUmCmGmAmAmAmGmAmUGUGCmCGmCAAmCGCUCUmGmCCmUmUmCmUGGCAUCG*mU*mU |
| (G021481) | | |
| ORF | 812 | ATGgaggccTcccccgccTccggcccccggcaccTgaTggacccccacaTcTTcaccTccAACTTCAACAACggcATCggccggCACAAGacc |
| encoding | | TACCTGTGCTACgaggTggagcggCTGGACAACggcaccTccgTgAAGATGGACCAGCACcggggcTTCCTGCACAACCAGgccAAGAACCTG |
| Sp. Cas9 | | CTGTGCggcTTCTACggccggCACgccgagCTGcggTTCCTGGACCTGgTgcccTccCTGCAGCTGGACcccgccCAGATCTACcgggTgacc |
| | | TGGTTCATCTCCTGGTCCCCCTGCTTCTccTGGggcTGCgccggcgaggTgcgggccTTCCTGCAGgagAACaccCACgTgcggCTGcggATC |
| | | TTCgccgcccggATCTACGACTACGACcccCTGTACAAGgaggccCTGCAGATGCTGcggGACgccggcgccCAGgTgTccATCATGaccTAC |
| | | GACgagTTCAAGCACTGCTGGGACaccTTCgTgGACCACCAGggcTGCcccTTCCAGcccTGGGACggcCTGGACgagCACTccCAGgccCTG |
| | | TccggccggCTGcgggccATCCTGCAGAACCAGggcAACTccggcTccgagacccccggcaccTccgagTccgccacccccgagTccgacaag |
| | | aagTacTccaTcggccTggCcaTcggcaccaacTccgTgggcTgggccgTgaTcaccgacgagTacaaggTgcccTccaagaagTTcaaggTg |
| | | cTgggcaacaccgaccggcacTccaTcaagaagaaccTgaTcggcgcccTgcTgTTcgacTccggcgagaccgccgaggccacccggcTgaag |
| | | cggaccgcccggcggcggTacacccggcggaagaaccggaTcTgcTaccTgcaggagaTcTTcTccaacgagaTggccaaggTggacgacTcc |
| | | TTCTTccaccggcTggaggagTccTTccTggTggaggaggacaagaagcacgagcggcaccccaTcTTcggcaacaTcgTggacgaggTggcc |
| | | TaccacgagaagTaccccaccaTcTaccaccTgcggaagaagcTggTggacTccaccgacaaggccgaccTgcggcTgaTcTaccTggcccTg |
| | | gcccacaTgaTcaagTTccggggccacTTccTgaTcgagggcgaccTgaaccccgacaacTccgacgTggacaagcTgTTcaTccagcTggTg |
| | | cagaccTacaaccagcTgTTcgaggagaaccccaTcaacgccTccggcgTggacgccaaggccaTccTgTccgcccggcTgTccaagTcccgg |
| | | cggcTggagaaccTgaTcgcccagcTgcccggcgagaagaagaacggccTgTTcggcaaccTgaTcgcccTgTcccTgggccTgacccccaac |
| | | TTcaagTccaacTTcgaccTggccgaggacgccaagcTgcagcTgTccaaggacaccTacgacgacgaccTggacaaccTgcTggcccagaTc |
| | | ggcgaccagTacgccgaccTgTTccTggccgccaagaaccTgTccgacgccaTccTgcTgTccgacaTccTgcgggTgaacaccgagaTcacc |
| | | aaggccccccTgTccgccTccaTgaTcaagcggTacgacgagcaccaccaggaccTgacccTgcTgaaggcccTggTgcggcagcagcTgccc |
| | | gagaagTacaaggagaTcTTcTTcgaccagTccaagaacggcTacgccggcTacaTcgacggcggcgccTcccaggaggagTTcTacaagTTc |
| | | aTcaagcccaTccTggagaagaTggacggcaccgaggagcTgcTggTgaagcTgaaccgggaggaccTgcTgcggaagcagcggaccTTcgac |
| | | aacggcTccaTcccccaccagaTccaccTgggcgagcTgcacgccaTccTgcggcggcaggaggacTTcTaccccTTccTgaaggacaaccgg |
| | | gagaagaTcgagaagaTccTgaccTTccggaTccccTacTacgTgggcccccTggcccggggcaacTcccggTTcgccTggaTgacccggaag |
| | | TccgaggagaccaTcacccccTggaacTTcgaggaggTggTggacaagggcgccTccgcccagTccTTcaTcgagcggaTgaccaacTTcgac |
| | | aagaaccTgcccaacgagaaggTgcTgcccaagcacTcccTgcTgTacgagTacTTcaccgTgTacaacgagcTgaccaaggTgaagTacgTg |
| | | accgagggcaTgcggaagcccgccTTccTgTccggcgagcagaagaaggccaTcgTggaccTgcTgTTcaagaccaaccggaaggTgaccgTg |
| | | aagcagcTgaaggaggacTacTTcaagaagaTcgagTgcTTcgacTccgTggagaTcTccggcgTggaggaccggTTcaacgccTcccTgggc |
| | | accTaccacgaccTgcTgaagaTcaTcaaggacaaggacTTccTggacaacgaggagaacgaggacaTccTggaggacaTcgTgcTgacccTg |
| | | acccTgTTcgaggaccgggagaTgaTcgaggagcggcTgaagaccTacgcccaccTgTTcgacgacaaggTgaTgaagcagcTgaagcggcgg |
| | | cggTacaccggcTggggccggcTgTcccggaagcTgaTcaacggcaTccgggacaagcagTccggcaagaccaTccTggacTTccTgaagTcc |
| | | gacggcTTcgccaaccggaacTTcaTgcagcTgaTccacgacgacTcccTgaccTTcaaggaggacaTccagaaggcccaggTgTccggccag |
| | | ggcgacTcccTgcacgagcacaTcgccaaccTggccggcTcccccgccaTcaagaagggcaTccTgcagaccgTgaaggTggTggacgagcTg |
| | | gTgaaggTgaTgggccggcacaagcccgagaacaTcgTgaTcgagaTggcccgggagaaccagaccacccagaagggccagaagaacTcccgg |
| | | gagcggaTgaagcggaTcgaggagggcaTcaaggagcTgggcTcccagaTccTgaaggagcaccccgTggagaacacccagcTgcagaacgag |
| | | aagcTgTaccTgTacTaccTgcagaacggccgggacaTgTacgTggaccaggagcTggacaTcaaccggcTgTccgacTacgacgTggaccac |
| | | aTcgTgccccagTccTTccTgaaggacgacTccaTcgacaacaaggTgcTgacccggTccgacaagaaccggggcaagTccgacaacgTgccc |
| | | TccgaggaggTggTgaagaagaTgaagaacTacTggcggcagcTgcTgaacgccaagcTgaTcacccagcggaagTTcgacaaccTgaccaag |
| | | gccgagcggggcggccTgTccgagcTggacaaggccggcTTcaTcaagcggcagcTggTggagacccggcagaTcaccaagcacgTggcccag |
| | | aTccTggacTcccggaTgaacaccaagTacgacgagaacgacaagcTgaTccgggaggTgaaggTgaTcacccTgaagTccaagcTggTgTcc |
| | | gacTTccggaaggacTTccagTTcTacaaggTgcgggagaTcaacaacTaccaccacgcccacgacgccTaccTgaacgccgTggTgggcacc |
| | | gcccTgaTcaagaagTaccccaagcTggagTccgagTTcgTgTacggcgacTacaaggTgTacgacgTgcggaagaTgaTcgccaagTccgag |
| | | caggagaTcggcaaggccaccgccaagTacTTcTTcTacTccaacaTcaTgaacTTcTTcaagaccgagaTcacccTggccaacggcgagaTc |
| | | cggaagcggccccTgaTcgagaccaacggcgagaccggcgagaTcgTgTgggacaagggccgggacTTcgccaccgTgcggaaggTgcTgTcc |
| | | aTgccccaggTgaacaTcgTgaagaagaccgaggTgcagaccggcggcTTcTccaaggagTccaTccTgcccaagcggaacTccgacaagcTg |
| | | aTcgcccggaagaaggacTgggaccccaagaagTacggcggcTTcgacTcccccaccgTggccTacTccgTgcTggTggTggccaaggTggag |
| | | aagggcaagTccaagaagcTgaagTccgTgaaggagcTgcTgggcaTcaccaTcaTggagcggTccTccTTcgagaagaaccccaTcgacTTc |
| | | cTggaggccaagggcTacaaggaggTgaagaaggaccTgaTcaTcaagcTgcccaagTacTcccTgTTcgagcTggagaacggccggaagcgg |
| | | aTgcTggccTccgccggcgagcTgcagaagggcaacgagcTggcccTgcccTccaagTacgTgaacTTccTgTaccTggccTcccacTacgag |
| | | aagcTgaagggcTcccccgaggacaacgagcagaagcagcTgTTcgTggagcagcacaagcacTaccTggacgagaTcaTcgagcagaTcTcc |
| | | gagTTcTccaagcgggTgaTccTggccgacgccaaccTggacaaggTgcTgTccgccTacaacaagcaccgggacaagcccaTccgggagcag |
| | | gccgagaacaTcaTccaccTgTTcacccTgaccaaccTgggcgcccccgccgccTTcaagTacTTcgacaccaccaTcgaccggaagcggTac |
| | | accTccaccaaggaggTgcTggacgccacccTgaTccaccagTccaTcaccggccTgTacgagacccggaTcgaccTgTcccagcTgggcggc |
| | | gacggcggcggcTcccccaagaagaagcggaaggTgTgA |
| ORF | 813 | ATGGACAAGAAGTACAGCATCGGACTGGACATCGGAACAAACAGCGTCGGATGGGCAGTCATCACAGACGAATACAAGGTCCCGAGCAAGAAG |
| encoding | | TTCAAGGTCCTGGGAAACACAGACAGACACAGCATCAAGAAGAACCTGATCGGAGCACTGCTGTTCGACAGCGGAGAAACAGCAGAAGCAACA |
| Sp. Cas9 | | AGACTGAAGAGAACAGCAAGAAGAAGATACACAAGAAGAAAGAACAGAATCTGCTACCTGCAGGAAATCTTCAGCAACGAAATGGCAAAGGTC |
| | | GACGACAGCTTCTTCCACAGACTGGAAGAAAGCTTCCTGGTCGAAGAAGACAAGAAGCACGAAAGACACCCGATCTTCGGAAACATCGTCGAC |
| | | GAAGTCGCATACCACGAAAAGTACCCGACAATCTACCACCTGAGAAAGAAGCTGGTCGACAGCACAGACAAGGCAGACCTGAGACTGATCTAC |
| | | CTGGCACTGGCACACATGATCAAGTTCAGAGGACACTTCCTGATCGAAGGAGACCTGAACCCGGACAACAGCGACGTCGACAAGCTGTTCATC |
| | | CAGCTGGTCCAGACATACAACCAGCTGTTCGAAGAAAACCCGATCAACGCAAGCGGAGTCGACGCAAAGGCAATCCTGAGCGCAAGACTGAGC |
| | | AAGAGCAGAAGACTGGAAAACCTGATCGCACAGCTGCCGGGAGAAAAGAAGAACGGACTGTTCGGAAACCTGATCGCACTGAGCCTGGGACTG |
| | | ACACCGAACTTCAAGAGCAACTTCGACCTGGCAGAAGACGCAAAGCTGCAGCTGAGCAAGGACACATACGACGACGACCTGGACAACCTGCTG |
| | | GCACAGATCGGAGACCAGTACGCAGACCTGTTCCTGGCAGCAAAGAACCTGAGCGACGCAATCCTGCTGAGCGACATCCTGAGAGTCAACACA |
| | | GAAATCACAAAGGCACCGCTGAGCGCAAGCATGATCAAGAGATACGACGAACACCACCAGGACCTGACACTGCTGAAGGCACTGGTCAGACAG |
| | | CAGCTGCCGGAAAAGTACAAGGAAATCTTCTTCGACCAGAGCAAGAACGGATACGCAGGATACATCGACGGAGGAGCAAGCCAGGAAGAATTC |
| | | TACAAGTTCATCAAGCCGATCCTGGAAAAGATGGACGGAACAGAAGAACTGCTGGTCAAGCTGAACAGAGAAGACCTGCTGAGAAAGCAGAGA |
| | | ACATTCGACAACGGAAGCATCCCGCACCAGATCCACCTGGGAGAACTGCACGCAATCCTGAGAAGACAGGAAGACTTCTACCCGTTCCTGAAG |
| | | GACAACAGAGAAAAGATCGAAAAGATCCTGACATTCAGAATCCCGTACTACGTCGGACCGCTGGCAAGAGGAAACAGCAGATTCGCATGGATG |
| | | ACAAGAAAGAGCGAAGAAACAATCACACCGTGGAACTTCGAAGAAGTCGTCGACAAGGGAGCAAGCGCACAGAGCTTCATCGAAAGAATGACA |
| | | AACTTCGACAAGAACCTGCCGAACGAAAAGGTCCTGCCGAAGCACAGCCTGCTGTACGAATACTTCACAGTCTACAACGAACTGACAAAGGTC |
| | | AAGTACGTCACAGAAGGAATGAGAAAGCCGGCATTCCTGAGCGGAGAACAGAAGAAGGCAATCGTCGACCTGCTGTTCAAGACAAACAGAAAG |
| | | GTCACAGTCAAGCAGCTGAAGGAAGACTACTTCAAGAAGATCGAATGCTTCGACAGCGTCGAAATCAGCGGAGTCGAAGACAGATTCAACGCA |
| | | AGCCTGGGAACATACCACGACCTGCTGAAGATCATCAAGGACAAGGACTTCCTGGACAACGAAGAAAACGAAGACATCCTGGAAGACATCGTC |
| | | CTGACACTGACACTGTTCGAAGACAGAGAAATGATCGAAGAAAGACTGAAGACATACGCACACCTGTTCGACGACAAGGTCATGAAGCAGCTG |
| | | AAGAGAAGAAGATACACAGGATGGGGAAGACTGAGCAGAAAGCTGATCAACGGAATCAGAGACAAGCAGAGCGGAAAGACAATCCTGGACTTC |
| | | CTGAAGAGCGACGGATTCGCAAACAGAAACTTCATGCAGCTGATCCACGACGACAGCCTGACATTCAAGGAAGACATCCAGAAGGCACAGGTC |
| | | AGCGGACAGGGAGACAGCCTGCACGAACACATCGCAAACCTGGCAGGAAGCCCGGCAATCAAGAAGGGAATCCTGCAGACAGTCAAGGTCGTC |
| | | GACGAACTGGTCAAGGTCATGGGAAGACACAAGCCGGAAAACATCGTCATCGAAATGGCAAGAGAAAACCAGACAACACAGAAGGGACAGAAG |
| | | AACAGCAGAGAAAGAATGAAGAGAATCGAAGAAGGAATCAAGGAACTGGGAAGCCAGATCCTGAAGGAACACCCGGTCGAAAACACACAGCTG |
| | | CAGAACGAAAAGCTGTACCTGTACTACCTGCAGAACGGAAGAGACATGTACGTCGACCAGGAACTGGACATCAACAGACTGAGCGACTACGAC |
| | | GTCGACCACATCGTCCCGCAGAGCTTCCTGAAGGACGACAGCATCGACAACAAGGTCCTGACAAGAAGCGACAAGAACAGAGGAAAGAGCGAC |
| | | AACGTCCCGAGCGAAGAAGTCGTCAAGAAGATGAAGAACTACTGGAGACAGCTGCTGAACGCAAAGCTGATCACACAGAGAAAGTTCGACAAC |
| | | CTGACAAAGGCAGAGAGAGGAGGACTGAGCGAACTGGACAAGGCAGGATTCATCAAGAGACAGCTGGTCGAAACAAGACAGATCACAAAGCAC |
| | | GTCGCACAGATCCTGGACAGCAGAATGAACACAAAGTACGACGAAAACGACAAGCTGATCAGAGAAGTCAAGGTCATCACACTGAAGAGCAAG |
| | | CTGGTCAGCGACTTCAGAAAGGACTTCCAGTTCTACAAGGTCAGAGAAATCAACAACTACCACCACGCACACGACGCATACCTGAACGCAGTC |
| | | GTCGGAACAGCACTGATCAAGAAGTACCCGAAGCTGGAAAGCGAATTCGTCTACGGAGACTACAAGGTCTACGACGTCAGAAAGATGATCGCA |
| | | AAGAGCGAACAGGAAATCGGAAAGGCAACAGCAAAGTACTTCTTCTACAGCAACATCATGAACTTCTTCAAGACAGAAATCACACTGGCAAAC |
| | | GGAGAAATCAGAAAGAGACCGCTGATCGAAACAAACGGAGAAACAGGAGAAATCGTCTGGGACAAGGGAAGAGACTTCGCAACAGTCAGAAAG |
| | | GTCCTGAGCATGCCGCAGGTCAACATCGTCAAGAAGACAGAAGTCCAGACAGGAGGATTCAGCAAGGAAAGCATCCTGCCGAAGAGAAACAGC |
| | | GACAAGCTGATCGCAAGAAAGAAGGACTGGGACCCGAAGAAGTACGGAGGATTCGACAGCCCGACAGTCGCATACAGCGTCCTGGTCGTCGCA |
| | | AAGGTCGAAAAGGGAAAGAGCAAGAAGCTGAAGAGCGTCAAGGAACTGCTGGGAATCACAATCATGGAAAGAAGCAGCTTCGAAAAGAACCCG |
| | | ATCGACTTCCTGGAAGCAAAGGGATACAAGGAAGTCAAGAAGGACCTGATCATCAAGCTGCCGAAGTACAGCCTGTTCGAACTGGAAAACGGA |
| | | AGAAAGAGAATGCTGGCAAGCGCAGGAGAACTGCAGAAGGGAAACGAACTGGCACTGCCGAGCAAGTACGTCAACTTCCTGTACCTGGCAAGC |
| | | CACTACGAAAAGCTGAAGGGAAGCCCGGAAGACAACGAACAGAAGCAGCTGTTCGTCGAACAGCACAAGCACTACCTGGACGAAATCATCGAA |
| | | CAGATCAGCGAATTCAGCAAGAGAGTCATCCTGGCAGACGCAAACCTGGACAAGGTCCTGAGCGCATACAACAAGCACAGAGACAAGCCGATC |
| | | AGAGAACAGGCAGAAAACATCATCCACCTGTTCACACTGACAAACCTGGGAGCACCGGCAGCATTCAAGTACTTCGACACAACAATCGACAGA |
| | | AAGAGATACACAAGCACAAAGGAAGTCCTGGACGCAACACTGATCCACCAGAGCATCACAGGACTGTACGAAACAAGAATCGACCTGAGCCAG |
| | | CTGGGAGGAGACGGAGGAGGAAGCCCGAAGAAGAAGAGAAAGGTCTAG |
| ORF | 814 | ATGGACAAGAAGTACTCCATCGGCCTGGACATCGGCACCAACTCCGTGGGCTGGGCCGTGATCACCGACGAGTACAAGGTGCCCTCCAAGAAG |
| encoding | | TTCAAGGTGCTGGGCAACACCGACCGGCACTCCATCAAGAAGAACCTGATCGGCGCCCTGCTGTTCGACTCCGGCGAGACCGCCGAGGCCACC |
| Sp. Cas9 | | CGGCTGAAGCGGACCGCCCGGCGGCGGTACACCCGGCGGAAGAACCGGATCTGCTACCTGCAGGAGATCTTCTCCAACGAGATGGCCAAGGTG |
| | | GACGACTCCTTCTTCCACCGGCTGGAGGAGTCCTTCCTGGTGGAGGAGGACAAGAAGCACGAGCGGCACCCCATCTTCGGCAACATCGTGGAC |
| | | GAGGTGGCCTACCACGAGAAGTACCCCACCATCTACCACCTGCGGAAGAAGCTGGTGGACTCCACCGACAAGGCCGACCTGCGGCTGATCTAC |
| | | CTGGCCCTGGCCCACATGATCAAGTTCCGGGGCCACTTCCTGATCGAGGGCGACCTGAACCCCGACAACTCCGACGTGGACAAGCTGTTCATC |
| | | CAGCTGGTGCAGACCTACAACCAGCTGTTCGAGGAGAACCCCATCAACGCCTCCGGCGTGGACGCCAAGGCCATCCTGTCCGCCCGGCTGTCC |
| | | AAGTCCCGGCGGCTGGAGAACCTGATCGCCCAGCTGCCCGGCGAGAAGAAGAACGGCCTGTTCGGCAACCTGATCGCCCTGTCCCTGGGCCTG |
| | | ACCCCCAACTTCAAGTCCAACTTCGACCTGGCCGAGGACGCCAAGCTGCAGCTGTCCAAGGACACCTACGACGACGACCTGGACAACCTGCTG |
| | | GCCCAGATCGGCGACCAGTACGCCGACCTGTTCCTGGCCGCCAAGAACCTGTCCGACGCCATCCTGCTGTCCGACATCCTGCGGGTGAACACC |
| | | GAGATCACCAAGGCCCCCCTGTCCGCCTCCATGATCAAGCGGTACGACGAGCACCACCAGGACCTGACCCTGCTGAAGGCCCTGGTGCGGCAG |
| | | CAGCTGCCCGAGAAGTACAAGGAGATCTTCTTCGACCAGTCCAAGAACGGCTACGCCGGCTACATCGACGGCGGCGCCTCCCAGGAGGAGTTC |
| | | TACAAGTTCATCAAGCCCATCCTGGAGAAGATGGACGGCACCGAGGAGCTGCTGGTGAAGCTGAACCGGGAGGACCTGCTGCGGAAGCAGCGG |
| | | ACCTTCGACAACGGCTCCATCCCCCACCAGATCCACCTGGGCGAGCTGCACGCCATCCTGCGGCGGCAGGAGGACTTCTACCCCTTCCTGAAG |
| | | GACAACCGGGAGAAGATCGAGAAGATCCTGACCTTCCGGATCCCCTACTACGTGGGCCCCCTGGCCCGGGGCAACTCCCGGTTCGCCTGGATG |
| | | ACCCGGAAGTCCGAGGAGACCATCACCCCCTGGAACTTCGAGGAGGTGGTGGACAAGGGCGCCTCCGCCCAGTCCTTCATCGAGCGGATGACC |
| | | AACTTCGACAAGAACCTGCCCAACGAGAAGGTGCTGCCCAAGCACTCCCTGCTGTACGAGTACTTCACCGTGTACAACGAGCTGACCAAGGTG |
| | | AAGTACGTGACCGAGGGCATGCGGAAGCCCGCCTTCCTGTCCGGCGAGCAGAAGAAGGCCATCGTGGACCTGCTGTTCAAGACCAACCGGAAG |
| | | GTGACCGTGAAGCAGCTGAAGGAGGACTACTTCAAGAAGATCGAGTGCTTCGACTCCGTGGAGATCTCCGGCGTGGAGGACCGGTTCAACGCC |
| | | TCCCTGGGCACCTACCACGACCTGCTGAAGATCATCAAGGACAAGGACTTCCTGGACAACGAGGAGAACGAGGACATCCTGGAGGACATCGTG |
| | | CTGACCCTGACCCTGTTCGAGGACCGGGAGATGATCGAGGAGCGGCTGAAGACCTACGCCCACCTGTTCGACGACAAGGTGATGAAGCAGCTG |
| | | AAGCGGCGGCGGTACACCGGCTGGGGCCGGCTGTCCCGGAAGCTGATCAACGGCATCCGGGACAAGCAGTCCGGCAAGACCATCCTGGACTTC |
| | | CTGAAGTCCGACGGCTTCGCCAACCGGAACTTCATGCAGCTGATCCACGACGACTCCCTGACCTTCAAGGAGGACATCCAGAAGGCCCAGGTG |
| | | TCCGGCCAGGGCGACTCCCTGCACGAGCACATCGCCAACCTGGCCGGCTCCCCCGCCATCAAGAAGGGCATCCTGCAGACCGTGAAGGTGGTG |
| | | GACGAGCTGGTGAAGGTGATGGGCCGGCACAAGCCCGAGAACATCGTGATCGAGATGGCCCGGGAGAACCAGACCACCCAGAAGGGCCAGAAG |
| | | AACTCCCGGGAGCGGATGAAGCGGATCGAGGAGGGCATCAAGGAGCTGGGCTCCCAGATCCTGAAGGAGCACCCCGTGGAGAACACCCAGCTG |
| | | CAGAACGAGAAGCTGTACCTGTACTACCTGCAGAACGGCCGGGACATGTACGTGGACCAGGAGCTGGACATCAACCGGCTGTCCGACTACGAC |
| | | GTGGACCACATCGTGCCCCAGTCCTTCCTGAAGGACGACTCCATCGACAACAAGGTGCTGACCCGGTCCGACAAGAACCGGGGCAAGTCCGAC |
| | | AACGTGCCCTCCGAGGAGGTGGTGAAGAAGATGAAGAACTACTGGCGGCAGCTGCTGAACGCCAAGCTGATCACCCAGCGGAAGTTCGACAAC |
| | | CTGACCAAGGCCGAGCGGGGCGGCCTGTCCGAGCTGGACAAGGCCGGCTTCATCAAGCGGCAGCTGGTGGAGACCCGGCAGATCACCAAGCAC |
| | | GTGGCCCAGATCCTGGACTCCCGGATGAACACCAAGTACGACGAGAACGACAAGCTGATCCGGGAGGTGAAGGTGATCACCCTGAAGTCCAAG |
| | | CTGGTGTCCGACTTCCGGAAGGACTTCCAGTTCTACAAGGTGCGGGAGATCAACAACTACCACCACGCCCACGACGCCTACCTGAACGCCGTG |
| | | GTGGGCACCGCCCTGATCAAGAAGTACCCCAAGCTGGAGTCCGAGTTCGTGTACGGCGACTACAAGGTGTACGACGTGCGGAAGATGATCGCC |
| | | AAGTCCGAGCAGGAGATCGGCAAGGCCACCGCCAAGTACTTCTTCTACTCCAACATCATGAACTTCTTCAAGACCGAGATCACCCTGGCCAAC |
| | | GGCGAGATCCGGAAGCGGCCCCTGATCGAGACCAACGGCGAGACCGGCGAGATCGTGTGGGACAAGGGCCGGGACTTCGCCACCGTGCGGAAG |
| | | GTGCTGTCCATGCCCCAGGTGAACATCGTGAAGAAGACCGAGGTGCAGACCGGCGGCTTCTCCAAGGAGTCCATCCTGCCCAAGCGGAACTCC |
| | | GACAAGCTGATCGCCCGGAAGAAGGACTGGGACCCCAAGAAGTACGGCGGCTTCGACTCCCCCACCGTGGCCTACTCCGTGCTGGTGGTGGCC |
| | | AAGGTGGAGAAGGGCAAGTCCAAGAAGCTGAAGTCCGTGAAGGAGCTGCTGGGCATCACCATCATGGAGCGGTCCTCCTTCGAGAAGAACCCC |
| | | ATCGACTTCCTGGAGGCCAAGGGCTACAAGGAGGTGAAGAAGGACCTGATCATCAAGCTGCCCAAGTACTCCCTGTTCGAGCTGGAGAACGGC |
| | | CGGAAGCGGATGCTGGCCTCCGCCGGCGAGCTGCAGAAGGGCAACGAGCTGGCCCTGCCCTCCAAGTACGTGAACTTCCTGTACCTGGCCTCC |
| | | CACTACGAGAAGCTGAAGGGCTCCCCCGAGGACAACGAGCAGAAGCAGCTGTTCGTGGAGCAGCACAAGCACTACCTGGACGAGATCATCGAG |
| | | CAGATCTCCGAGTTCTCCAAGCGGGTGATCCTGGCCGACGCCAACCTGGACAAGGTGCTGTCCGCCTACAACAAGCACCGGGACAAGCCCATC |
| | | CGGGAGCAGGCCGAGAACATCATCCACCTGTTCACCCTGACCAACCTGGGCGCCCCCGCCGCCTTCAAGTACTTCGACACCACCATCGACCGG |
| | | AAGCGGTACACCTCCACCAAGGAGGTGCTGGACGCCACCCTGATCCACCAGTCCATCACCGGCCTGTACGAGACCCGGATCGACCTGTCCCAG |
| | | CTGGGCGGCGACGGCGGCGGCTCCCCCAAGAAGAAGCGGAAGGTGTGA |
| ORF | 815 | AUGGACAAGAAGUACAGCAUCGGCCUGGACAUCGGCACGAACAGCGUUGGCUGGGCUGUGAUCACGGACGAGUACAAGGUUCCCUCAAAGAAG |
| encoding | | UUCAAGGUGCUGGGCAACACGGACCGGCACAGCAUCAAGAAGAAUCUCAUCGGUGCACUGCUGUUCGACAGCGGUGAGACGGCCGAAGCCACG |
| Sp. Cas9 | | CGGCUGAAGCGGACGGCCCGCCGGCGGUACACGCGGCGGAAGAACCGGAUCUGCUACCUGCAGGAGAUCUUCAGCAACGAGAUGGCCAAGGUG |
| | | GACGACAGCUUCUUCCACCGGCUGGAGGAGAGCUUCCUGGUGGAGGAGGACAAGAAGCACGAGCGGCACCCCAUCUUCGGCAACAUCGUGGAC |
| | | GAAGUCGCCUACCACGAGAAGUACCCCACCAUCUACCACCUGCGGAAGAAGCUGGUGGACUCGACUGACAAGGCCGACCUGCGGCUGAUCUAC |
| | | CUGGCACUGGCCCACAUGAUAAAGUUCCGGGGCCACUUCCUGAUCGAGGGCGACCUGAACCCUGACAACAGCGACGUGGACAAGCUGUUCAUC |
| | | CAGCUGGUGCAGACCUACAACCAGCUGUUCGAGGAGAACCCCAUCAACGCCAGCGGCGUGGACGCCAAGGCCAUCCUCAGCGCCCGCCUCAGC |
| | | AAGAGCCGGCGGCUGGAGAAUCUCAUCGCCCAGCUUCCAGGUGAGAAGAAGAAUGGGCUGUUCGGCAAUCUCAUCGCACUCAGCCUGGGCCUG |
| | | ACUCCCAACUUCAAGAGCAACUUCGACCUGGCCGAGGACGCCAAGCUGCAGCUCAGCAAGGACACCUACGACGACGACCUGGACAAUCUCCUG |
| | | GCCCAGAUCGGCGACCAGUACGCCGACCUGUUCCUGGCUGCCAAGAAUCUCAGCGACGCCAUCCUGCUCAGCGACAUCCUGCGGGUGAACACA |
| | | GAGAUCACGAAGGCCCCCCUCAGCGCCAGCAUGAUAAAGCGGUACGACGAGCACCACCAGGACCUGACGCUGCUGAAGGCACUGGUGCGGCAG |
| | | CAGCUUCCAGAGAAGUACAAGGAGAUCUUCUUCGACCAGAGCAAGAAUGGGUACGCCGGGUACAUCGACGGUGGUGCCAGCCAGGAGGAGUUC |
| | | UACAAGUUCAUCAAGCCCAUCCUGGAGAAGAUGGACGGCACAGAGGAGCUGCUGGUGAAGCUGAACAGGGAGGACCUGCUGCGGAAGCAGCGG |
| | | ACGUUCGACAAUGGGAGCAUCCCCCACCAGAUCCACCUGGGUGAGCUGCACGCCAUCCUGCGGCGGCAGGAGGACUUCUACCCCUUCCUGAAG |
| | | GACAACAGGGAGAAGAUCGAGAAGAUCCUGACGUUCCGGAUCCCCUACUACGUUGGCCCCCUGGCCCGCGGCAACAGCCGGUUCGCCUGGAUG |
| | | ACGCGGAAGAGCGAGGAGACGAUCACUCCCUGGAACUUCGAGGAAGUCGUGGACAAGGGUGCCAGCGCCCAGAGCUUCAUCGAGCGGAUGACG |
| | | AACUUCGACAAGAAUCUUCCAAACGAGAAGGUGCUUCCAAAGCACAGCCUGCUGUACGAGUACUUCACGGUGUACAACGAGCUGACGAAGGUG |
| | | AAGUACGUGACAGAGGGCAUGCGGAAGCCCGCCUUCCUCAGCGGUGAGCAGAAGAAGGCCAUCGUGGACCUGCUGUUCAAGACGAACCGGAAG |
| | | GUGACGGUGAAGCAGCUGAAGGAGGACUACUUCAAGAAGAUCGAGUGCUUCGACAGCGUGGAGAUCAGCGGCGUGGAGGACCGGUUCAACGCC |
| | | AGCCUGGGCACCUACCACGACCUGCUGAAGAUCAUCAAGGACAAGGACUUCCUGGACAACGAGGAGAACGAGGACAUCCUGGAGGACAUCGUG |
| | | CUGACGCUGACGCUGUUCGAGGACAGGGAGAUGAUAGAGGAGCGGCUGAAGACCUACGCCCACCUGUUCGACGACAAGGUGAUGAAGCAGCUG |
| | | AAGCGGCGGCGGUACACGGGCUGGGGCCGGCUCAGCCGGAAGCUGAUCAAUGGGAUCCGAGACAAGCAGAGCGGCAAGACGAUCCUGGACUUC |
| | | CUGAAGAGCGACGGCUUCGCCAACCGGAACUUCAUGCAGCUGAUCCACGACGACAGCCUGACGUUCAAGGAGGACAUCCAGAAGGCCCAGGUC |
| | | AGCGGCCAGGGCGACAGCCUGCACGAGCACAUCGCCAAUCUCGCCGGGAGCCCCGCCAUCAAGAAGGGGAUCCUGCAGACGGUGAAGGUGGUG |
| | | GACGAGCUGGUGAAGGUGAUGGGCCGGCACAAGCCAGAGAACAUCGUGAUCGAGAUGGCCAGGGAGAACCAGACGACUCAAAAGGGGCAGAAG |
| | | AACAGCAGGGAGCGGAUGAAGCGGAUCGAGGAGGGCAUCAAGGAGCUGGGCAGCCAGAUCCUGAAGGAGCACCCCGUGGAGAACACUCAACUG |
| | | CAGAACGAGAAGCUGUACCUGUACUACCUGCAGAAUGGGCGAGACAUGUACGUGGACCAGGAGCUGGACAUCAACCGGCUCAGCGACUACGAC |
| | | GUGGACCACAUCGUUCCCCAGAGCUUCCUGAAGGACGACAGCAUCGACAACAAGGUGCUGACGCGGAGCGACAAGAACCGGGGCAAGAGCGAC |
| | | AACGUUCCCUCAGAGGAAGUCGUGAAGAAGAUGAAGAACUACUGGCGGCAGCUGCUGAACGCCAAGCUGAUCACUCAACGGAAGUUCGACAAU |
| | | CUCACGAAGGCCGAGCGGGGUGGCCUCAGCGAGCUGGACAAGGCCGGGUUCAUCAAGCGGCAGCUGGUGGAGACGCGGCAGAUCACGAAGCAC |
| | | GUGGCCCAGAUCCUGGACAGCCGGAUGAACACGAAGUACGACGAGAACGACAAGCUGAUCAGGGAAGUCAAGGUGAUCACGCUGAAGAGCAAG |
| | | CUGGUCAGCGACUUCCGGAAGGACUUCCAGUUCUACAAGGUGAGGGAGAUCAACAACUACCACCACGCCCACGACGCCUACCUGAACGCUGUG |
| | | GUUGGCACGGCACUGAUCAAGAAGUACCCCAAGCUGGAGAGCGAGUUCGUGUACGGCGACUACAAGGUGUACGACGUGCGGAAGAUGAUAGCC |
| | | AAGAGCGAGCAGGAGAUCGGCAAGGCCACGGCCAAGUACUUCUUCUACAGCAACAUCAUGAACUUCUUCAAGACAGAGAUCACGCUGGCCAAU |
| | | GGUGAGAUCCGGAAGCGGCCCCUGAUCGAGACGAAUGGUGAGACGGGUGAGAUCGUGUGGGACAAGGGGCGAGACUUCGCCACGGUGCGGAAG |
| | | GUGCUCAGCAUGCCCCAGGUGAACAUCGUGAAGAAGACAGAAGUCCAGACGGGUGGCUUCAGCAAGGAGAGCAUCCUUCCAAAGCGGAACAGC |
| | | GACAAGCUGAUCGCCCGCAAGAAGGACUGGGACCCCAAGAAGUACGGUGGCUUCGACAGCCCCACCGUGGCCUACAGCGUGCUGGUGGUGGCC |
| | | AAGGUGGAGAAGGGGAAGAGCAAGAAGCUGAAGAGCGUGAAGGAGCUGCUGGGCAUCACGAUCAUGGAGCGGAGCAGCUUCGAGAAGAACCCC |
| | | AUCGACUUCCUGGAAGCCAAGGGGUACAAGGAAGUCAAGAAGGACCUGAUCAUCAAGCUUCCAAAGUACAGCCUGUUCGAGCUGGAGAAUGGG |
| | | CGGAAGCGGAUGCUGGCCAGCGCCGGUGAGCUGCAGAAGGGGAACGAGCUGGCACUUCCCUCAAAGUACGUGAACUUCCUGUACCUGGCCAGC |
| | | CACUACGAGAAGCUGAAGGGGAGCCCAGAGGACAACGAGCAGAAGCAGCUGUUCGUGGAGCAGCACAAGCACUACCUGGACGAGAUCAUCGAG |
| | | CAGAUCAGCGAGUUCAGCAAGCGGGUGAUCCUGGCCGACGCCAAUCUCGACAAGGUGCUCAGCGCCUACAACAAGCACCGAGACAAGCCCAUC |
| | | AGGGAGCAGGCCGAGAACAUCAUCCACCUGUUCACGCUGACGAAUCUCGGUGCCCCCGCUGCCUUCAAGUACUUCGACACGACGAUCGACCGG |
| | | AAGCGGUACACGUCGACUAAGGAAGUCCUGGACGCCACGCUGAUCCACCAGAGCAUCACGGGCCUGUACGAGACGCGGAUCGACCUCAGCCAG |
| | | CUGGGUGGCGACGGUGGUGGCAGCCCCAAGAAGAAGCGGAAGGUGUAG |
| ORF | 816 | AUGGACAAGAAGUACAGCAUCGGCCUCGACAUCGGCACCAACAGCGUCGGCUGGGCCGUCAUCACCGACGAGUACAAGGUCCCCAGCAAGAAG |
| encoding | | UUCAAGGUCCUCGGCAACACCGACCGCCACAGCAUCAAGAAGAACCUCAUCGGCGCCCUCCUCUUCGACAGCGGCGAGACCGCCGAGGCCACC |
| Sp. Cas9 | | CGCCUCAAGCGCACCGCCCGCCGCCGCUACACCCGCCGCAAGAACCGCAUCUGCUACCUCCAGGAGAUCUUCAGCAACGAGAUGGCCAAGGUC |
| | | GACGACAGCUUCUUCCACCGCCUCGAGGAGAGCUUCCUCGUCGAGGAGGACAAGAAGCACGAGCGCCACCCCAUCUUCGGCAACAUCGUCGAC |
| | | GAGGUCGCCUACCACGAGAAGUACCCCACCAUCUACCACCUCCGCAAGAAGCUCGUCGACAGCACCGACAAGGCCGACCUCCGCCUCAUCUAC |
| | | CUCGCCCUCGCCCACAUGAUCAAGUUCCGCGGCCACUUCCUCAUCGAGGGCGACCUCAACCCCGACAACAGCGACGUCGACAAGCUCUUCAUC |
| | | CAGCUCGUCCAGACCUACAACCAGCUCUUCGAGGAGAACCCCAUCAACGCCAGCGGCGUCGACGCCAAGGCCAUCCUCAGCGCCCGCCUCAGC |
| | | AAGAGCCGCCGCCUCGAGAACCUCAUCGCCCAGCUCCCCGGCGAGAAGAAGAACGGCCUCUUCGGCAACCUCAUCGCCCUCAGCCUCGGCCUC |
| | | ACCCCCAACUUCAAGAGCAACUUCGACCUCGCCGAGGACGCCAAGCUCCAGCUCAGCAAGGACACCUACGACGACGACCUCGACAACCUCCUC |
| | | GCCCAGAUCGGCGACCAGUACGCCGACCUCUUCCUCGCCGCCAAGAACCUCAGCGACGCCAUCCUCCUCAGCGACAUCCUCCGCGUCAACACC |
| | | GAGAUCACCAAGGCCCCCCUCAGCGCCAGCAUGAUCAAGCGCUACGACGAGCACCACCAGGACCUCACCCUCCUCAAGGCCCUCGUCCGCCAG |
| | | CAGCUCCCCGAGAAGUACAAGGAGAUCUUCUUCGACCAGAGCAAGAACGGCUACGCCGGCUACAUCGACGGCGGCGCCAGCCAGGAGGAGUUC |
| | | UACAAGUUCAUCAAGCCCAUCCUCGAGAAGAUGGACGGCACCGAGGAGCUCCUCGUCAAGCUCAACCGCGAGGACCUCCUCCGCAAGCAGCGC |
| | | ACCUUCGACAACGGCAGCAUCCCCCACCAGAUCCACCUCGGCGAGCUCCACGCCAUCCUCCGCCGCCAGGAGGACUUCUACCCCUUCCUCAAG |
| | | GACAACCGCGAGAAGAUCGAGAAGAUCCUCACCUUCCGCAUCCCCUACUACGUCGGCCCCCUCGCCCGCGGCAACAGCCGCUUCGCCUGGAUG |
| | | ACCCGCAAGAGCGAGGAGACCAUCACCCCCUGGAACUUCGAGGAGGUCGUCGACAAGGGCGCCAGCGCCCAGAGCUUCAUCGAGCGCAUGACC |
| | | AACUUCGACAAGAACCUCCCCAACGAGAAGGUCCUCCCCAAGCACAGCCUCCUCUACGAGUACUUCACCGUCUACAACGAGCUCACCAAGGUC |
| | | AAGUACGUCACCGAGGGCAUGCGCAAGCCCGCCUUCCUCAGCGGCGAGCAGAAGAAGGCCAUCGUCGACCUCCUCUUCAAGACCAACCGCAAG |
| | | GUCACCGUCAAGCAGCUCAAGGAGGACUACUUCAAGAAGAUCGAGUGCUUCGACAGCGUCGAGAUCAGCGGCGUCGAGGACCGCUUCAACGCC |
| | | AGCCUCGGCACCUACCACGACCUCCUCAAGAUCAUCAAGGACAAGGACUUCCUCGACAACGAGGAGAACGAGGACAUCCUCGAGGACAUCGUC |
| | | CUCACCCUCACCCUCUUCGAGGACCGCGAGAUGAUCGAGGAGCGCCUCAAGACCUACGCCCACCUCUUCGACGACAAGGUCAUGAAGCAGCUC |
| | | AAGCGCCGCCGCUACACCGGCUGGGGCCGCCUCAGCCGCAAGCUCAUCAACGGCAUCCGCGACAAGCAGAGCGGCAAGACCAUCCUCGACUUC |
| | | CUCAAGAGCGACGGCUUCGCCAACCGCAACUUCAUGCAGCUCAUCCACGACGACAGCCUCACCUUCAAGGAGGACAUCCAGAAGGCCCAGGUC |
| | | AGCGGCCAGGGCGACAGCCUCCACGAGCACAUCGCCAACCUCGCCGGCAGCCCCGCCAUCAAGAAGGGCAUCCUCCAGACCGUCAAGGUCGUC |
| | | GACGAGCUCGUCAAGGUCAUGGGCCGCCACAAGCCCGAGAACAUCGUCAUCGAGAUGGCCCGCGAGAACCAGACCACCCAGAAGGGCCAGAAG |
| | | AACAGCCGCGAGCGCAUGAAGCGCAUCGAGGAGGGCAUCAAGGAGCUCGGCAGCCAGAUCCUCAAGGAGCACCCCGUCGAGAACACCCAGCUC |
| | | CAGAACGAGAAGCUCUACCUCUACUACCUCCAGAACGGCCGCGACAUGUACGUCGACCAGGAGCUCGACAUCAACCGCCUCAGCGACUACGAC |
| | | GUCGACCACAUCGUCCCCCAGAGCUUCCUCAAGGACGACAGCAUCGACAACAAGGUCCUCACCCGCAGCGACAAGAACCGCGGCAAGAGCGAC |
| | | AACGUCCCCAGCGAGGAGGUCGUCAAGAAGAUGAAGAACUACUGGCGCCAGCUCCUCAACGCCAAGCUCAUCACCCAGCGCAAGUUCGACAAC |
| | | CUCACCAAGGCCGAGCGCGGCGGCCUCAGCGAGCUCGACAAGGCCGGCUUCAUCAAGCGCCAGCUCGUCGAGACCCGCCAGAUCACCAAGCAC |
| | | GUCGCCCAGAUCCUCGACAGCCGCAUGAACACCAAGUACGACGAGAACGACAAGCUCAUCCGCGAGGUCAAGGUCAUCACCCUCAAGAGCAAG |
| | | CUCGUCAGCGACUUCCGCAAGGACUUCCAGUUCUACAAGGUCCGCGAGAUCAACAACUACCACCACGCCCACGACGCCUACCUCAACGCCGUC |
| | | GUCGGCACCGCCCUCAUCAAGAAGUACCCCAAGCUCGAGAGCGAGUUCGUCUACGGCGACUACAAGGUCUACGACGUCCGCAAGAUGAUCGCC |
| | | AAGAGCGAGCAGGAGAUCGGCAAGGCCACCGCCAAGUACUUCUUCUACAGCAACAUCAUGAACUUCUUCAAGACCGAGAUCACCCUCGCCAAC |
| | | GGCGAGAUCCGCAAGCGCCCCCUCAUCGAGACCAACGGCGAGACCGGCGAGAUCGUCUGGGACAAGGGCCGCGACUUCGCCACCGUCCGCAAG |
| | | GUCCUCAGCAUGCCCCAGGUCAACAUCGUCAAGAAGACCGAGGUCCAGACCGGCGGCUUCAGCAAGGAGAGCAUCCUCCCCAAGCGCAACAGC |
| | | GACAAGCUCAUCGCCCGCAAGAAGGACUGGGACCCCAAGAAGUACGGCGGCUUCGACAGCCCCACCGUCGCCUACAGCGUCCUCGUCGUCGCC |
| | | AAGGUCGAGAAGGGCAAGAGCAAGAAGCUCAAGAGCGUCAAGGAGCUCCUCGGCAUCACCAUCAUGGAGCGCAGCAGCUUCGAGAAGAACCCC |
| | | AUCGACUUCCUCGAGGCCAAGGGCUACAAGGAGGUCAAGAAGGACCUCAUCAUCAAGCUCCCCAAGUACAGCCUCUUCGAGCUCGAGAACGGC |
| | | CGCAAGCGCAUGCUCGCCAGCGCCGGCGAGCUCCAGAAGGGCAACGAGCUCGCCCUCCCCAGCAAGUACGUCAACUUCCUCUACCUCGCCAGC |
| | | CACUACGAGAAGCUCAAGGGCAGCCCCGAGGACAACGAGCAGAAGCAGCUCUUCGUCGAGCAGCACAAGCACUACCUCGACGAGAUCAUCGAG |
| | | CAGAUCAGCGAGUUCAGCAAGCGCGUCAUCCUCGCCGACGCCAACCUCGACAAGGUCCUCAGCGCCUACAACAAGCACCGCGACAAGCCCAUC |
| | | CGCGAGCAGGCCGAGAACAUCAUCCACCUCUUCACCCUCACCAACCUCGGCGCCCCCGCCGCCUUCAAGUACUUCGACACCACCAUCGACCGC |
| | | AAGCGCUACACCAGCACCAAGGAGGUCCUCGACGCCACCCUCAUCCACCAGAGCAUCACCGGCCUCUACGAGACCCGCAUCGACCUCAGCCAG |
| | | CUCGGCGGCGACGGCGGCGGCAGCCCCAAGAAGAAGCGCAAGGUCUAG |
| ORF | 817 | ATGGACAAGAAGTACAGCATCGGCCTGGACATCGGCACCAACAGCGTGGGCTGGGCCGTGATCACCGACGAGTACAAGGTGCCCAGCAAGAAG |
| encoding | | TTCAAGGTGCTGGGCAACACCGACCGGCACAGCATCAAGAAGAACCTGATCGGCGCCCTGCTGTTCGACAGCGGCGAGACCGCCGAGGCCACC |
| Sp. Cas9 | | CGGCTGAAGCGGACCGCCCGGCGGCGGTACACCCGGCGGAAGAACCGGATCTGCTACCTGCAGGAGATCTTCAGCAACGAGATGGCCAAGGTG |
| | | GACGACAGCTTCTTCCACCGGCTGGAGGAGAGCTTCCTGGTGGAGGAGGACAAGAAGCACGAGCGGCACCCCATCTTCGGCAACATCGTGGAC |
| | | GAGGTGGCCTACCACGAGAAGTACCCCACCATCTACCACCTGCGGAAGAAGCTGGTGGACAGCACCGACAAGGCCGACCTGCGGCTGATCTAC |
| | | CTGGCCCTGGCCCACATGATCAAGTTCCGGGGCCACTTCCTGATCGAGGGCGACCTGAACCCCGACAACAGCGACGTGGACAAGCTGTTCATC |
| | | CAGCTGGTGCAGACCTACAACCAGCTGTTCGAGGAGAACCCCATCAACGCCAGCGGCGTGGACGCCAAGGCCATCCTGAGCGCCCGGCTGAGC |
| | | AAGAGCCGGCGGCTGGAGAACCTGATCGCCCAGCTGCCCGGCGAGAAGAAGAACGGCCTGTTCGGCAACCTGATCGCCCTGAGCCTGGGCCTG |
| | | ACCCCCAACTTCAAGAGCAACTTCGACCTGGCCGAGGACGCCAAGCTGCAGCTGAGCAAGGACACCTACGACGACGACCTGGACAACCTGCTG |
| | | GCCCAGATCGGCGACCAGTACGCCGACCTGTTCCTGGCCGCCAAGAACCTGAGCGACGCCATCCTGCTGAGCGACATCCTGCGGGTGAACACC |
| | | GAGATCACCAAGGCCCCCCTGAGCGCCAGCATGATCAAGCGGTACGACGAGCACCACCAGGACCTGACCCTGCTGAAGGCCCTGGTGCGGCAG |
| | | CAGCTGCCCGAGAAGTACAAGGAGATCTTCTTCGACCAGAGCAAGAACGGCTACGCCGGCTACATCGACGGCGGCGCCAGCCAGGAGGAGTTC |
| | | TACAAGTTCATCAAGCCCATCCTGGAGAAGATGGACGGCACCGAGGAGCTGCTGGTGAAGCTGAACCGGGAGGACCTGCTGCGGAAGCAGCGG |
| | | ACCTTCGACAACGGCAGCATCCCCCACCAGATCCACCTGGGCGAGCTGCACGCCATCCTGCGGCGGCAGGAGGACTTCTACCCCTTCCTGAAG |
| | | GACAACCGGGAGAAGATCGAGAAGATCCTGACCTTCCGGATCCCCTACTACGTGGGCCCCCTGGCCCGGGGCAACAGCCGGTTCGCCTGGATG |
| | | ACCCGGAAGAGCGAGGAGACCATCACCCCCTGGAACTTCGAGGAGGTGGTGGACAAGGGCGCCAGCGCCCAGAGCTTCATCGAGCGGATGACC |
| | | AACTTCGACAAGAACCTGCCCAACGAGAAGGTGCTGCCCAAGCACAGCCTGCTGTACGAGTACTTCACCGTGTACAACGAGCTGACCAAGGTG |
| | | AAGTACGTGACCGAGGGCATGCGGAAGCCCGCCTTCCTGAGCGGCGAGCAGAAGAAGGCCATCGTGGACCTGCTGTTCAAGACCAACCGGAAG |
| | | GTGACCGTGAAGCAGCTGAAGGAGGACTACTTCAAGAAGATCGAGTGCTTCGACAGCGTGGAGATCAGCGGCGTGGAGGACCGGTTCAACGCC |
| | | AGCCTGGGCACCTACCACGACCTGCTGAAGATCATCAAGGACAAGGACTTCCTGGACAACGAGGAGAACGAGGACATCCTGGAGGACATCGTG |
| | | CTGACCCTGACCCTGTTCGAGGACCGGGAGATGATCGAGGAGCGGCTGAAGACCTACGCCCACCTGTTCGACGACAAGGTGATGAAGCAGCTG |
| | | AAGCGGCGGCGGTACACCGGCTGGGGCCGGCTGAGCCGGAAGCTGATCAACGGCATCCGGGACAAGCAGAGCGGCAAGACCATCCTGGACTTC |
| | | CTGAAGAGCGACGGCTTCGCCAACCGGAACTTCATGCAGCTGATCCACGACGACAGCCTGACCTTCAAGGAGGACATCCAGAAGGCCCAGGTG |
| | | AGCGGCCAGGGCGACAGCCTGCACGAGCACATCGCCAACCTGGCCGGCAGCCCCGCCATCAAGAAGGGCATCCTGCAGACCGTGAAGGTGGTG |
| | | GACGAGCTGGTGAAGGTGATGGGCCGGCACAAGCCCGAGAACATCGTGATCGAGATGGCCCGGGAGAACCAGACCACCCAGAAGGGCCAGAAG |
| | | AACAGCCGGGAGCGGATGAAGCGGATCGAGGAGGGCATCAAGGAGCTGGGCAGCCAGATCCTGAAGGAGCACCCCGTGGAGAACACCCAGCTG |
| | | CAGAACGAGAAGCTGTACCTGTACTACCTGCAGAACGGCCGGGACATGTACGTGGACCAGGAGCTGGACATCAACCGGCTGAGCGACTACGAC |
| | | GTGGACCACATCGTGCCCCAGAGCTTCCTGAAGGACGACAGCATCGACAACAAGGTGCTGACCCGGAGCGACAAGAACCGGGGCAAGAGCGAC |
| | | AACGTGCCCAGCGAGGAGGTGGTGAAGAAGATGAAGAACTACTGGCGGCAGCTGCTGAACGCCAAGCTGATCACCCAGCGGAAGTTCGACAAC |
| | | CTGACCAAGGCCGAGCGGGGCGGCCTGAGCGAGCTGGACAAGGCCGGCTTCATCAAGCGGCAGCTGGTGGAGACCCGGCAGATCACCAAGCAC |
| | | GTGGCCCAGATCCTGGACAGCCGGATGAACACCAAGTACGACGAGAACGACAAGCTGATCCGGGAGGTGAAGGTGATCACCCTGAAGAGCAAG |
| | | CTGGTGAGCGACTTCCGGAAGGACTTCCAGTTCTACAAGGTGCGGGAGATCAACAACTACCACCACGCCCACGACGCCTACCTGAACGCCGTG |
| | | GTGGGCACCGCCCTGATCAAGAAGTACCCCAAGCTGGAGAGCGAGTTCGTGTACGGCGACTACAAGGTGTACGACGTGCGGAAGATGATCGCC |
| | | AAGAGCGAGCAGGAGATCGGCAAGGCCACCGCCAAGTACTTCTTCTACAGCAACATCATGAACTTCTTCAAGACCGAGATCACCCTGGCCAAC |
| | | GGCGAGATCCGGAAGCGGCCCCTGATCGAGACCAACGGCGAGACCGGCGAGATCGTGTGGGACAAGGGCCGGGACTTCGCCACCGTGCGGAAG |
| | | GTGCTGAGCATGCCCCAGGTGAACATCGTGAAGAAGACCGAGGTGCAGACCGGCGGCTTCAGCAAGGAGAGCATCCTGCCCAAGCGGAACAGC |
| | | GACAAGCTGATCGCCCGGAAGAAGGACTGGGACCCCAAGAAGTACGGCGGCTTCGACAGCCCCACCGTGGCCTACAGCGTGCTGGTGGTGGCC |
| | | AAGGTGGAGAAGGGCAAGAGCAAGAAGCTGAAGAGCGTGAAGGAGCTGCTGGGCATCACCATCATGGAGCGGAGCAGCTTCGAGAAGAACCCC |
| | | ATCGACTTCCTGGAGGCCAAGGGCTACAAGGAGGTGAAGAAGGACCTGATCATCAAGCTGCCCAAGTACAGCCTGTTCGAGCTGGAGAACGGC |
| | | CGGAAGCGGATGCTGGCCAGCGCCGGCGAGCTGCAGAAGGGCAACGAGCTGGCCCTGCCCAGCAAGTACGTGAACTTCCTGTACCTGGCCAGC |
| | | CACTACGAGAAGCTGAAGGGCAGCCCCGAGGACAACGAGCAGAAGCAGCTGTTCGTGGAGCAGCACAAGCACTACCTGGACGAGATCATCGAG |
| | | CAGATCAGCGAGTTCAGCAAGCGGGTGATCCTGGCCGACGCCAACCTGGACAAGGTGCTGAGCGCCTACAACAAGCACCGGGACAAGCCCATC |
| | | CGGGAGCAGGCCGAGAACATCATCCACCTGTTCACCCTGACCAACCTGGGCGCCCCCGCCGCCTTCAAGTACTTCGACACCACCATCGACCGG |
| | | AAGCGGTACACCAGCACCAAGGAGGTGCTGGACGCCACCCTGATCCACCAGAGCATCACCGGCCTGTACGAGACCCGGATCGACCTGAGCCAG |
| | | CTGGGCGGCGACGGCGGCGGCAGCCCCAAGAAGAAGCGGAAGGTGTGA |
| amino acid | 818 | MDKKYSIGLDIGTNSVGWAVITDEYKVPSKKFKVLGNTDRHSIKKNLIGALLFDSGETAEATRLKRTARRRYTRRKNRICYLQEIFSNEMAKV |
| sequence | | DDSFFHRLEESFLVEEDKKHERHPIFGNIVDEVAYHEKYPTIYHLRKKLVDSTDKADLRLIYLALAHMIKFRGHFLIEGDLNPDNSDVDKLFI |
| for Sp. | | QLVQTYNQLFEENPINASGVDAKAILSARLSKSRRLENLIAQLPGEKKNGLFGNLIALSLGLTPNFKSNFDLAEDAKLQLSKDTYDDDLDNLL |
| Cas9 | | AQIGDQYADLFLAAKNLSDAILLSDILRVNTEITKAPLSASMIKRYDEHHQDLTLLKALVRQQLPEKYKEIFFDQSKNGYAGYIDGGASQEEF |
| | | YKFIKPILEKMDGTEELLVKLNREDLLRKQRTFDNGSIPHQIHLGELHAILRRQEDFYPFLKDNREKIEKILTFRIPYYVGPLARGNSRFAWM |
| | | TRKSEETITPWNFEEVVDKGASAQSFIERMTNFDKNLPNEKVLPKHSLLYEYFTVYNELTKVKYVTEGMRKPAFLSGEQKKAIVDLLFKTNRK |
| | | VTVKQLKEDYFKKIECFDSVEISGVEDRFNASLGTYHDLLKIIKDKDFLDNEENEDILEDIVLTLTLFEDREMIEERLKTYAHLFDDKVMKQL |
| | | KRRRYTGWGRLSRKLINGIRDKQSGKTILDFLKSDGFANRNFMQLIHDDSLTFKEDIQKAQVSGQGDSLHEHIANLAGSPAIKKGILQTVKVV |
| | | DELVKVMGRHKPENIVIEMARENQTTQKGQKNSRERMKRIEEGIKELGSQILKEHPVENTQLQNEKLYLYYLONGRDMYVDQELDINRLSDYD |
| | | VDHIVPQSFLKDDSIDNKVLTRSDKNRGKSDNVPSEEVVKKMKNYWRQLLNAKLITQRKFDNLTKAERGGLSELDKAGFIKRQLVETRQITKH |
| | | VAQILDSRMNTKYDENDKLIREVKVITLKSKLVSDFRKDFQFYKVREINNYHHAHDAYLNAVVGTALIKKYPKLESEFVYGDYKVYDVRKMIA |
| | | KSEQEIGKATAKYFFYSNIMNFFKTEITLANGEIRKRPLIETNGETGEIVWDKGRDFATVRKVLSMPQVNIVKKTEVQTGGFSKESILPKRNS |
| | | DKLIARKKDWDPKKYGGFDSPTVAYSVLVVAKVEKGKSKKLKSVKELLGITIMERSSFEKNPIDFLEAKGYKEVKKDLIIKLPKYSLFELENG |
| | | RKRMLASAGELQKGNELALPSKYVNFLYLASHYEKLKGSPEDNEQKQLFVEQHKHYLDEIIEQISEFSKRVILADANLDKVLSAYNKHRDKPI |
| | | REQAENIIHLFTLTNLGAPAAFKYFDTTIDRKRYTSTKEVLDATLIHQSITGLYETRIDLSQLGGDGGGSPKKKRKV |
| Open | 819 | AUGGACAAGAAGUACUCCAUCGGCCUGGACAUCGGCACCAACUCCGUGGGCUGGGCCGUGAUCACCGACGAGUACAAGGUGCCCUCCAAGAAG |
| reading | | UUCAAGGUGCUGGGCAACACCGACCGGCACUCCAUCAAGAAGAACCUGAUCGGCGCCCUGCUGUUCGACUCCGGCGAGACCGCCGAGGCCACC |
| frame for | | CGGCUGAAGCGGACCGCCCGGCGGCGGUACACCCGGCGGAAGAACCGGAUCUGCUACCUGCAGGAGAUCUUCUCCAACGAGAUGGCCAAGGUG |
| Cas9 with | | GACGACUCCUUCUUCCACCGGCUGGAGGAGUCCUUCCUGGUGGAGGAGGACAAGAAGCACGAGCGGCACCCCAUCUUCGGCAACAUCGUGGAC |
| Hibit tag | | GAGGUGGCCUACCACGAGAAGUACCCCACCAUCUACCACCUGCGGAAGAAGCUGGUGGACUCCACCGACAAGGCCGACCUGCGGCUGAUCUAC |
| | | CUGGCCCUGGCCCACAUGAUCAAGUUCCGGGGCCACUUCCUGAUCGAGGGCGACCUGAACCCCGACAACUCCGACGUGGACAAGCUGUUCAUC |
| | | CAGCUGGUGCAGACCUACAACCAGCUGUUCGAGGAGAACCCCAUCAACGCCUCCGGCGUGGACGCCAAGGCCAUCCUGUCCGCCCGGCUGUCC |
| | | AAGUCCCGGCGGCUGGAGAACCUGAUCGCCCAGCUGCCCGGCGAGAAGAAGAACGGCCUGUUCGGCAACCUGAUCGCCCUGUCCCUGGGCCUG |
| | | ACCCCCAACUUCAAGUCCAACUUCGACCUGGCCGAGGACGCCAAGCUGCAGCUGUCCAAGGACACCUACGACGACGACCUGGACAACCUGCUG |
| | | GCCCAGAUCGGCGACCAGUACGCCGACCUGUUCCUGGCCGCCAAGAACCUGUCCGACGCCAUCCUGCUGUCCGACAUCCUGCGGGUGAACACC |
| | | GAGAUCACCAAGGCCCCCCUGUCCGCCUCCAUGAUCAAGCGGUACGACGAGCACCACCAGGACCUGACCCUGCUGAAGGCCCUGGUGCGGCAG |
| | | CAGCUGCCCGAGAAGUACAAGGAGAUCUUCUUCGACCAGUCCAAGAACGGCUACGCCGGCUACAUCGACGGCGGCGCCUCCCAGGAGGAGUUC |
| | | UACAAGUUCAUCAAGCCCAUCCUGGAGAAGAUGGACGGCACCGAGGAGCUGCUGGUGAAGCUGAACCGGGAGGACCUGCUGCGGAAGCAGCGG |
| | | ACCUUCGACAACGGCUCCAUCCCCCACCAGAUCCACCUGGGCGAGCUGCACGCCAUCCUGCGGCGGCAGGAGGACUUCUACCCCUUCCUGAAG |
| | | GACAACCGGGAGAAGAUCGAGAAGAUCCUGACCUUCCGGAUCCCCUACUACGUGGGCCCCCUGGCCCGGGGCAACUCCCGGUUCGCCUGGAUG |
| | | ACCCGGAAGUCCGAGGAGACCAUCACCCCCUGGAACUUCGAGGAGGUGGUGGACAAGGGCGCCUCCGCCCAGUCCUUCAUCGAGCGGAUGACC |
| | | AACUUCGACAAGAACCUGCCCAACGAGAAGGUGCUGCCCAAGCACUCCCUGCUGUACGAGUACUUCACCGUGUACAACGAGCUGACCAAGGUG |
| | | AAGUACGUGACCGAGGGCAUGCGGAAGCCCGCCUUCCUGUCCGGCGAGCAGAAGAAGGCCAUCGUGGACCUGCUGUUCAAGACCAACCGGAAG |
| | | GUGACCGUGAAGCAGCUGAAGGAGGACUACUUCAAGAAGAUCGAGUGCUUCGACUCCGUGGAGAUCUCCGGCGUGGAGGACCGGUUCAACGCC |
| | | UCCCUGGGCACCUACCACGACCUGCUGAAGAUCAUCAAGGACAAGGACUUCCUGGACAACGAGGAGAACGAGGACAUCCUGGAGGACAUCGUG |
| | | CUGACCCUGACCCUGUUCGAGGACCGGGAGAUGAUCGAGGAGCGGCUGAAGACCUACGCCCACCUGUUCGACGACAAGGUGAUGAAGCAGCUG |
| | | AAGCGGCGGCGGUACACCGGCUGGGGCCGGCUGUCCCGGAAGCUGAUCAACGGCAUCCGGGACAAGCAGUCCGGCAAGACCAUCCUGGACUUC |
| | | CUGAAGUCCGACGGCUUCGCCAACCGGAACUUCAUGCAGCUGAUCCACGACGACUCCCUGACCUUCAAGGAGGACAUCCAGAAGGCCCAGGUG |
| | | UCCGGCCAGGGCGACUCCCUGCACGAGCACAUCGCCAACCUGGCCGGCUCCCCCGCCAUCAAGAAGGGCAUCCUGCAGACCGUGAAGGUGGUG |
| | | GACGAGCUGGUGAAGGUGAUGGGCCGGCACAAGCCCGAGAACAUCGUGAUCGAGAUGGCCCGGGAGAACCAGACCACCCAGAAGGGCCAGAAG |
| | | AACUCCCGGGAGCGGAUGAAGCGGAUCGAGGAGGGCAUCAAGGAGCUGGGCUCCCAGAUCCUGAAGGAGCACCCCGUGGAGAACACCCAGCUG |
| | | CAGAACGAGAAGCUGUACCUGUACUACCUGCAGAACGGCCGGGACAUGUACGUGGACCAGGAGCUGGACAUCAACCGGCUGUCCGACUACGAC |
| | | GUGGACCACAUCGUGCCCCAGUCCUUCCUGAAGGACGACUCCAUCGACAACAAGGUGCUGACCCGGUCCGACAAGAACCGGGGCAAGUCCGAC |
| | | AACGUGCCCUCCGAGGAGGUGGUGAAGAAGAUGAAGAACUACUGGCGGCAGCUGCUGAACGCCAAGCUGAUCACCCAGCGGAAGUUCGACAAC |
| | | CUGACCAAGGCCGAGCGGGGCGGCCUGUCCGAGCUGGACAAGGCCGGCUUCAUCAAGCGGCAGCUGGUGGAGACCCGGCAGAUCACCAAGCAC |
| | | GUGGCCCAGAUCCUGGACUCCCGGAUGAACACCAAGUACGACGAGAACGACAAGCUGAUCCGGGAGGUGAAGGUGAUCACCCUGAAGUCCAAG |
| | | CUGGUGUCCGACUUCCGGAAGGACUUCCAGUUCUACAAGGUGCGGGAGAUCAACAACUACCACCACGCCCACGACGCCUACCUGAACGCCGUG |
| | | GUGGGCACCGCCCUGAUCAAGAAGUACCCCAAGCUGGAGUCCGAGUUCGUGUACGGCGACUACAAGGUGUACGACGUGCGGAAGAUGAUCGCC |
| | | AAGUCCGAGCAGGAGAUCGGCAAGGCCACCGCCAAGUACUUCUUCUACUCCAACAUCAUGAACUUCUUCAAGACCGAGAUCACCCUGGCCAAC |
| | | GGCGAGAUCCGGAAGCGGCCCCUGAUCGAGACCAACGGCGAGACCGGCGAGAUCGUGUGGGACAAGGGCCGGGACUUCGCCACCGUGCGGAAG |
| | | GUGCUGUCCAUGCCCCAGGUGAACAUCGUGAAGAAGACCGAGGUGCAGACCGGCGGCUUCUCCAAGGAGUCCAUCCUGCCCAAGCGGAACUCC |
| | | GACAAGCUGAUCGCCCGGAAGAAGGACUGGGACCCCAAGAAGUACGGCGGCUUCGACUCCCCCACCGUGGCCUACUCCGUGCUGGUGGUGGCC |
| | | AAGGUGGAGAAGGGCAAGUCCAAGAAGCUGAAGUCCGUGAAGGAGCUGCUGGGCAUCACCAUCAUGGAGCGGUCCUCCUUCGAGAAGAACCCC |
| | | AUCGACUUCCUGGAGGCCAAGGGCUACAAGGAGGUGAAGAAGGACCUGAUCAUCAAGCUGCCCAAGUACUCCCUGUUCGAGCUGGAGAACGGC |
| | | CGGAAGCGGAUGCUGGCCUCCGCCGGCGAGCUGCAGAAGGGCAACGAGCUGGCCCUGCCCUCCAAGUACGUGAACUUCCUGUACCUGGCCUCC |
| | | CACUACGAGAAGCUGAAGGGCUCCCCCGAGGACAACGAGCAGAAGCAGCUGUUCGUGGAGCAGCACAAGCACUACCUGGACGAGAUCAUCGAG |
| | | CAGAUCUCCGAGUUCUCCAAGCGGGUGAUCCUGGCCGACGCCAACCUGGACAAGGUGCUGUCCGCCUACAACAAGCACCGGGACAAGCCCAUC |
| | | CGGGAGCAGGCCGAGAACAUCAUCCACCUGUUCACCCUGACCAACCUGGGCGCCCCCGCCGCCUUCAAGUACUUCGACACCACCAUCGACCGG |
| | | AAGCGGUACACCUCCACCAAGGAGGUGCUGGACGCCACCCUGAUCCACCAGUCCAUCACCGGCCUGUACGAGACCCGGAUCGACCUGUCCCAG |
| | | CUGGGCGGCGACGGCGGCGGCUCCCCCAAGAAGAAGCGGAAGGUGUCCGAGUCCGCCACCCCCGAGUCCGUGUCCGGCUGGCGGCUGUUCAAG |
| | | AAGAUCUCCUGA |
| Amino acid | 820 | MDKKYSIGLDIGTNSVGWAVITDEYKVPSKKFKVLGNTDRHSIKKNLIGALLFDSGETAEATRLKRTARRRYTRRKNRICYLQEIFSNEMAKV |
| sequence | | DDSFFHRLEESFLVEEDKKHERHPIFGNIVDEVAYHEKYPTIYHLRKKLVDSTDKADLRLIYLALAHMIKFRGHFLIEGDLNPDNSDVDKLFI |
| for Cas9 | | QLVQTYNQLFEENPINASGVDAKAILSARLSKSRRLENLIAQLPGEKKNGLFGNLIALSLGLTPNFKSNEDLAEDAKLQLSKDTYDDDLDNLL |
| with Hibit | | AQIGDQYADLFLAAKNLSDAILLSDILRVNTEITKAPLSASMIKRYDEHHQDLTLLKALVRQQLPEKYKEIFFDQSKNGYAGYIDGGASQEEF |
| tag | | YKFIKPILEKMDGTEELLVKLNREDLLRKQRTFDNGSIPHQIHLGELHAILRRQEDFYPFLKDNREKIEKILTFRIPYYVGPLARGNSRFAWM |
| | | TRKSEETITPWNFEEVVDKGASAQSFIERMTNFDKNLPNEKVLPKHSLLYEYFTVYNELTKVKYVTEGMRKPAFLSGEQKKAIVDLLFKTNRK |
| | | VTVKQLKEDYFKKIECFDSVEISGVEDRFNASLGTYHDLLKIIKDKDFLDNEENEDILEDIVLTLTLFEDREMIEERLKTYAHLFDDKVMKQL |
| | | KRRRYTGWGRLSRKLINGIRDKQSGKTILDFLKSDGFANRNFMQLIHDDSLTFKEDIQKAQVSGQGDSLHEHIANLAGSPAIKKGILQTVKVV |
| | | DELVKVMGRHKPENIVIEMARENQTTQKGQKNSRERMKRIEEGIKELGSQILKEHPVENTQLQNEKLYLYYLONGRDMYVDQELDINRLSDYD |
| | | VDHIVPQSFLKDDSIDNKVLTRSDKNRGKSDNVPSEEVVKKMKNYWRQLLNAKLITQRKFDNLTKAERGGLSELDKAGFIKRQLVETRQITKH |
| | | VAQILDSRMNTKYDENDKLIREVKVITLKSKLVSDFRKDFQFYKVREINNYHHAHDAYLNAVVGTALIKKYPKLESEFVYGDYKVYDVRKMIA |
| | | KSEQEIGKATAKYFFYSNIMNFFKTEITLANGEIRKRPLIETNGETGEIVWDKGRDFATVRKVLSMPQVNIVKKTEVQTGGFSKESILPKRNS |
| | | DKLIARKKDWDPKKYGGFDSPTVAYSVLVVAKVEKGKSKKLKSVKELLGITIMERSSFEKNPIDELEAKGYKEVKKDLIIKLPKYSLFELENG |
| | | RKRMLASAGELQKGNELALPSKYVNFLYLASHYEKLKGSPEDNEQKQLFVEQHKHYLDEIIEQISEFSKRVILADANLDKVLSAYNKHRDKPI |
| | | REQAENIIHLFTLTNLGAPAAFKYFDTTIDRKRYTSTKEVLDATLIHQSITGLYETRIDLSQLGGDGGGSPKKKRKVSESATPESVSGWRLFK |
| | | KIS |
| mRNA | 821 | GGGAAGCUCAGAAUAAACGCUCAACUUUGGCCGGAUCUGCCACCAUGACCAACCUGUCCGACAUCAUCGAGAAGGAGACCGGCAAGCAGCUGG |
| encoding | | UGAUCCAGGAGUCCAUCCUGAUGCUGCCCGAGGAGGUGGAGGAGGUGAUCGGCAACAAGCCCGAGUCCGACAUCCUGGUGCACACCGCCUACG |
| UGI | | ACGAGUCCACCGACGAGAACGUGAUGCUGCUGACCUCCGACGCCCCCGAGUACAAGCCCUGGGCCCUGGUGAUCCAGGACUCCAACGGCGAGA |
| | | ACAAGAUCAAGAUGCUGUCCGGCGGCUCCAAGCGGACCGCCGACGGCUCCGAGUUCGAGUCCCCCAAGAAGAAGCGGAAGGUGGAGUGAUAGC |
| | | UAGCACCAGCCUCAAGAACACCCGAAUGGAGUCUCUAAGCUACAUAAUACCAACUUACACUUUACAAAAUGUUGUCCCCCAAAAUGUAGCCAU |
| | | UCGUAUCUGCUCCUAAUAAAAAGAAAGUUUCUUCACAUUCUCUCGAGAAAAAAAAAAAAUGGAAAAAAAAAAAACGGAAAAAAAAAAAAGGUA |
| | | AAAAAAAAAAAUAUAAAAAAAAAAAACAUAAAAAAAAAAAACGAAAAAAAAAAAACGUAAAAAAAAAAAACUCAAAAAAAAAAAAGAUAAAAA |
| | | AAAAAAACCUAAAAAAAAAAAAUGUAAAAAAAAAAAAGGGAAAAAAAAAAAACGCAAAAAAAAAAAACACAAAAAAAAAAAAUGCAAAAAAAA |
| | | AAAAUCGAAAAAAAAAAAAUCUAAAAAAAAAAAACGAAAAAAAAAAAACCCAAAAAAAAAAAAGACAAAAAAAAAAAAUAGAAAAAAAAAAAA |
| | | GUUAAAAAAAAAAAACUGAAAAAAAAAAAAUUUAAAAAAAAAAAAUCUAG |
| mRNA | 822 | GGGAAGCUCAGAAUAAACGCUCAACUUUGGCCGGAUCUGCCACCAUGGACGGCUCCGGCGGCGGCUCCCCCAAGAAGAAGCGGAAGGUGGAGG |
| encoding Nme2Cas9 | | ACAAGCGGCCCGCCGCCACCAAGAAGGCCGGCCAGGCCAAGAAGAAGAAGGGCGGCUCCGGCGGCGGCGAGGCCUCCCCCGCCUCCGGCCCCC |
| base editor | | GGCACCUGAUGGACCCCCACAUCUUCACCUCCAACUUCAACAACGGCAUCGGCCGGCACAAGACCUACCUGUGCUACGAGGUGGAGCGGCUGG |
| | | ACAACGGCACCUCCGUGAAGAUGGACCAGCACCGGGGCUUCCUGCACAACCAGGCCAAGAACCUGCUGUGCGGCUUCUACGGCCGGCACGCCG |
| | | AGCUGCGGUUCCUGGACCUGGUGCCCUCCCUGCAGCUGGACCCCGCCCAGAUCUACCGGGUGACCUGGUUCAUCUCCUGGUCCCCCUGCUUCU |
| | | CCUGGGGCUGCGCCGGCGAGGUGCGGGCCUUCCUGCAGGAGAACACCCACGUGCGGCUGCGGAUCUUCGCCGCCCGGAUCUACGACUACGACC |
| | | CCCUGUACAAGGAGGCCCUGCAGAUGCUGCGGGACGCCGGCGCCCAGGUGUCCAUCAUGACCUACGACGAGUUCAAGCACUGCUGGGACACCU |
| | | UCGUGGACCACCAGGGCUGCCCCUUCCAGCCCUGGGACGGCCUGGACGAGCACUCCCAGGCCCUGUCCGGCCGGCUGCGGGCCAUCCUGCAGA |
| | | ACCAGGGCAACUCCGGCUCCGAGACCCCCGGCACCUCCGAGUCCGCCACCCCCGAGUCCGCAGCGUUCAAACCAAAUCCCAUCAACUACAUCC |
| | | UGGGCCUGGCCAUCGGCAUCGCCUCCGUGGGCUGGGCCAUGGUGGAGAUCGACGAGGAGGAGAACCCCAUCCGGCUGAUCGACCUGGGCGUGC |
| | | GGGUGUUCGAGCGGGCCGAGGUGCCCAAGACCGGCGACUCCCUGGCCAUGGCCCGGCGGCUGGCCCGGUCCGUGCGGCGGCUGACCCGGCGGC |
| | | GGGCCCACCGGCUGCUGCGGGCCCGGCGGCUGCUGAAGCGGGAGGGCGUGCUGCAGGCCGCCGACUUCGACGAGAACGGCCUGAUCAAGUCCC |
| | | UGCCCAACACCCCCUGGCAGCUGCGGGCCGCCGCCCUGGACCGGAAGCUGACCCCCCUGGAGUGGUCCGCCGUGCUGCUGCACCUGAUCAAGC |
| | | ACCGGGGCUACCUGUCCCAGCGGAAGAACGAGGGCGAGACCGCCGACAAGGAGCUGGGCGCCCUGCUGAAGGGCGUGGCCAACAACGCCCACG |
| | | CCCUGCAGACCGGCGACUUCCGGACCCCCGCCGAGCUGGCCCUGAACAAGUUCGAGAAGGAGUCCGGCCACAUCCGGAACCAGCGGGGCGACU |
| | | ACUCCCACACCUUCUCCCGGAAGGACCUGCAGGCCGAGCUGAUCCUGCUGUUCGAGAAGCAGAAGGAGUUCGGCAACCCCCACGUGUCCGGCG |
| | | GCCUGAAGGAGGGCAUCGAGACCCUGCUGAUGACCCAGCGGCCCGCCCUGUCCGGCGACGCCGUGCAGAAGAUGCUGGGCCACUGCACCUUCG |
| | | AGCCCGCCGAGCCCAAGGCCGCCAAGAACACCUACACCGCCGAGCGGUUCAUCUGGCUGACCAAGCUGAACAACCUGCGGAUCCUGGAGCAGG |
| | | GCUCCGAGCGGCCCCUGACCGACACCGAGCGGGCCACCCUGAUGGACGAGCCCUACCGGAAGUCCAAGCUGACCUACGCCCAGGCCCGGAAGC |
| | | UGCUGGGCCUGGAGGACACCGCCUUCUUCAAGGGCCUGCGGUACGGCAAGGACAACGCCGAGGCCUCCACCCUGAUGGAGAUGAAGGCCUACC |
| | | ACGCCAUCUCCCGGGCCCUGGAGAAGGAGGGCCUGAAGGACAAGAAGUCCCCCCUGAACCUGUCCUCCGAGCUGCAGGACGAGAUCGGCACCG |
| | | CCUUCUCCCUGUUCAAGACCGACGAGGACAUCACCGGCCGGCUGAAGGACCGGGUGCAGCCCGAGAUCCUGGAGGCCCUGCUGAAGCACAUCU |
| | | CCUUCGACAAGUUCGUGCAGAUCUCCCUGAAGGCCCUGCGGCGGAUCGUGCCCCUGAUGGAGCAGGGCAAGCGGUACGACGAGGCCUGCGCCG |
| | | AGAUCUACGGCGACCACUACGGCAAGAAGAACACCGAGGAGAAGAUCUACCUGCCCCCCAUCCCCGCCGACGAGAUCCGGAACCCCGUGGUGC |
| | | UGCGGGCCCUGUCCCAGGCCCGGAAGGUGAUCAACGGCGUGGUGCGGCGGUACGGCUCCCCCGCCCGGAUCCACAUCGAGACCGCCCGGGAGG |
| | | UGGGCAAGUCCUUCAAGGACCGGAAGGAGAUCGAGAAGCGGCAGGAGGAGAACCGGAAGGACCGGGAGAAGGCCGCCGCCAAGUUCCGGGAGU |
| | | ACUUCCCCAACUUCGUGGGCGAGCCCAAGUCCAAGGACAUCCUGAAGCUGCGGCUGUACGAGCAGCAGCACGGCAAGUGCCUGUACUCCGGCA |
| | | AGGAGAUCAACCUGGUGCGGCUGAACGAGAAGGGCUACGUGGAGAUCGACCACGCCCUGCCCUUCUCCCGGACCUGGGACGACUCCUUCAACA |
| | | ACAAGGUGCUGGUGCUGGGCUCCGAGAACCAGAACAAGGGCAACCAGACCCCCUACGAGUACUUCAACGGCAAGGACAACUCCCGGGAGUGGC |
| | | AGGAGUUCAAGGCCCGGGUGGAGACCUCCCGGUUCCCCCGGUCCAAGAAGCAGCGGAUCCUGCUGCAGAAGUUCGACGAGGACGGCUUCAAGG |
| | | AGUGCAACCUGAACGACACCCGGUACGUGAACCGCUUCCUGUGCCAGUUCGUGGCCGACCACAUCCUGCUGACCGGCAAGGGCAAGCGGCGGG |
| | | UGUUCGCCUCCAACGGCCAGAUCACCAACCUGCUGCGGGGCUUCUGGGGCCUGCGGAAGGUGCGGGCCGAGAACGACCGGCACCACGCCCUGG |
| | | ACGCCGUGGUGGUGGCCUGCUCCACCGUGGCCAUGCAGCAGAAGAUCACCCGGUUCGUGCGGUACAAGGAGAUGAACGCCUUCGACGGCAAGA |
| | | CCAUCGACAAGGAGACCGGCAAGGUGCUGCACCAGAAGACCCACUUCCCCCAGCCCUGGGAGUUCUUCGCCCAGGAGGUGAUGAUCCGGGUGU |
| | | UCGGCAAGCCCGACGGCAAGCCCGAGUUCGAGGAGGCCGACACCCCCGAGAAGCUGCGGACCCUGCUGGCCGAGAAGCUGUCCUCCCGGCCCG |
| | | AGGCCGUGCACGAGUACGUGACCCCCCUGUUCGUGUCCCGGGCCCCCAACCGGAAGAUGUCCGGCGCCCACAAGGACACCCUGCGGUCCGCCA |
| | | AGCGGUUCGUGAAGCACAACGAGAAGAUCUCCGUGAAGCGGGUGUGGCUGACCGAGAUCAAGCUGGCCGACCUGGAGAACAUGGUGAACUACA |
| | | AGAACGGCCGGGAGAUCGAGCUGUACGAGGCCCUGAAGGCCCGGCUGGAGGCCUACGGCGGCAACGCCAAGCAGGCCUUCGACCCCAAGGACA |
| | | ACCCCUUCUACAAGAAGGGCGGCCAGCUGGUGAAGGCCGUGCGGGUGGAGAAGACCCAGGAGUCCGGCGUGCUGCUGAACAAGAAGAACGCCU |
| | | ACACCAUCGCCGACAACGGCGACAUGGUGCGGGUGGACGUGUUCUGCAAGGUGGACAAGAAGGGCAAGAACCAGUACUUCAUCGUGCCCAUCU |
| | | ACGCCUGGCAGGUGGCCGAGAACAUCCUGCCCGACAUCGACUGCAAGGGCUACCGGAUCGACGACUCCUACACCUUCUGCUUCUCCCUGCACA |
| | | AGUACGACCUGAUCGCCUUCCAGAAGGACGAGAAGUCCAAGGUGGAGUUCGCCUACUACAUCAACUGCGACUCCUCCAACGGCCGGUUCUACC |
| | | UGGCCUGGCACGACAAGGGCUCCAAGGAGCAGCAGUUCCGGAUCUCCACCCAGAACCUGGUGCUGAUCCAGAAGUACCAGGUGAACGAGCUGG |
| | | GCAAGGAGAUCCGGCCCUGCCGGCUGAAGAAGCGGCCCCCCGUGCGGUAGUGACUAGCACCAGCCUCAAGAACACCCGAAUGGAGUCUCUAAG |
| | | CUACAUAAUACCAACUUACACUUUACAAAAUGUUGUCCCCCAAAAUGUAGCCAUUCGUAUCUGCUCCUAAUAAAAAGAAAGUUUCUUCACAUU |
| | | CUCUCGAGAAAAAAAAAAAAUGGAAAAAAAAAAAACGGAAAAAAAAAAAGGUAAAAAAAAAAAAUAUAAAAAAAAAAACAUAAAAAAAAAAAA |
| | | CGAAAAAAAAAAAACGUAAAAAAAAAAAACUCAAAAAAAAAAAGAUAAAAAAAAAAAACCUAAAAAAAAAAAAUGUAAAAAAAAAAAAGGGAA |
| | | AAAAAAAAACGCAAAAAAAAAAAACACAAAAAAAAAAAAUGCAAAAAAAAAAAAUCGAAAAAAAAAAAAUCUAAAAAAAAAAAACGAAAAAAA |
| | | AAAAACCCAAAAAAAAAAAAGACAAAAAAAAAAAAUAGAAAAAAAAAAAGUUAAAAAAAAAAAACUGAAAAAAAAAAAAUUUAAAAAAAAAAA |
| | | AUCUAG |
| Open | 824 | ATGACCAACCTGTCCGACATCATCGAGAAGGAGACCGGCAAGCAGCTGGTGATCCAGGAGTCCATCCTGATGCTGCCCGAGGAGGTGGAGGAG |
| reading frame for | | GTGATCGGCAACAAGCCCGAGTCCGACATCCTGGTGCACACCGCCTACGACGAGTCCACCGACGAGAACGTGATGCTGCTGACCTCCGACGCC |
| UGI encoded | | CCCGAGTACAAGCCCTGGGCCCTGGTGATCCAGGACTCCAACGGCGAGAACAAGATCAAGATGCTGTCCGGCGGCTCCAAGCGGACCGCCGAC |
| by SEQ ID NO: 821 | | GGCTCCGAGTTCGAGTCCCCCAAGAAGAAGCGGAAGGTGGAGTGATAG |
| MRNA | 825 | GGGAAGCUCAGAAUAAACGCUCAACUUUGGCCGGAUCUGCCACCAUGGACGGCUCCGGCGGCGGCUCCCCCAAGAAGAAGCGGAAGGUGGAGG |
| encoding | | ACAAGCGGCCCGCCGCCACCAAGAAGGCCGGCCAGGCCAAGAAGAAGAAGGGCGGCUCCGGCGGCGGCGCCGCCUUCAAGCCCAACCCCAUCA |
| Nme2 Cas9 | | ACUACAUCCUGGGCCUGGACAUCGGCAUCGCCUCCGUGGGCUGGGCCAUGGUGGAGAUCGACGAGGAGGAGAACCCCAUCCGGCUGAUCGACC |
| | | UGGGCGUGCGGGUGUUCGAGCGGGCCGAGGUGCCCAAGACCGGCGACUCCCUGGCCAUGGCCCGGCGGCUGGCCCGGUCCGUGCGGCGGCUGA |
| | | CCCGGCGGCGGGCCCACCGGCUGCUGCGGGCCCGGCGGCUGCUGAAGCGGGAGGGCGUGCUGCAGGCCGCCGACUUCGACGAGAACGGCCUGA |
| | | UCAAGUCCCUGCCCAACACCCCCUGGCAGCUGCGGGCCGCCGCCCUGGACCGGAAGCUGACCCCCCUGGAGUGGUCCGCCGUGCUGCUGCACC |
| | | UGAUCAAGCACCGGGGCUACCUGUCCCAGCGGAAGAACGAGGGCGAGACCGCCGACAAGGAGCUGGGCGCCCUGCUGAAGGGCGUGGCCAACA |
| | | ACGCCCACGCCCUGCAGACCGGCGACUUCCGGACCCCCGCCGAGCUGGCCCUGAACAAGUUCGAGAAGGAGUCCGGCCACAUCCGGAACCAGC |
| | | GGGGCGACUACUCCCACACCUUCUCCCGGAAGGACCUGCAGGCCGAGCUGAUCCUGCUGUUCGAGAAGCAGAAGGAGUUCGGCAACCCCCACG |
| | | UGUCCGGCGGCCUGAAGGAGGGCAUCGAGACCCUGCUGAUGACCCAGCGGCCCGCCCUGUCCGGCGACGCCGUGCAGAAGAUGCUGGGCCACU |
| | | GCACCUUCGAGCCCGCCGAGCCCAAGGCCGCCAAGAACACCUACACCGCCGAGCGGUUCAUCUGGCUGACCAAGCUGAACAACCUGCGGAUCC |
| | | UGGAGCAGGGCUCCGAGCGGCCCCUGACCGACACCGAGCGGGCCACCCUGAUGGACGAGCCCUACCGGAAGUCCAAGCUGACCUACGCCCAGG |
| | | CCCGGAAGCUGCUGGGCCUGGAGGACACCGCCUUCUUCAAGGGCCUGCGGUACGGCAAGGACAACGCCGAGGCCUCCACCCUGAUGGAGAUGA |
| | | AGGCCUACCACGCCAUCUCCCGGGCCCUGGAGAAGGAGGGCCUGAAGGACAAGAAGUCCCCCCUGAACCUGUCCUCCGAGCUGCAGGACGAGA |
| | | UCGGCACCGCCUUCUCCCUGUUCAAGACCGACGAGGACAUCACCGGCCGGCUGAAGGACCGGGUGCAGCCCGAGAUCCUGGAGGCCCUGCUGA |
| | | AGCACAUCUCCUUCGACAAGUUCGUGCAGAUCUCCCUGAAGGCCCUGCGGCGGAUCGUGCCCCUGAUGGAGCAGGGCAAGCGGUACGACGAGG |
| | | CCUGCGCCGAGAUCUACGGCGACCACUACGGCAAGAAGAACACCGAGGAGAAGAUCUACCUGCCCCCCAUCCCCGCCGACGAGAUCCGGAACC |
| | | CCGUGGUGCUGCGGGCCCUGUCCCAGGCCCGGAAGGUGAUCAACGGCGUGGUGCGGCGGUACGGCUCCCCCGCCCGGAUCCACAUCGAGACCG |
| | | CCCGGGAGGUGGGCAAGUCCUUCAAGGACCGGAAGGAGAUCGAGAAGCGGCAGGAGGAGAACCGGAAGGACCGGGAGAAGGCCGCCGCCAAGU |
| | | UCCGGGAGUACUUCCCCAACUUCGUGGGCGAGCCCAAGUCCAAGGACAUCCUGAAGCUGCGGCUGUACGAGCAGCAGCACGGCAAGUGCCUGU |
| | | ACUCCGGCAAGGAGAUCAACCUGGUGCGGCUGAACGAGAAGGGCUACGUGGAGAUCGACCACGCCCUGCCCUUCUCCCGGACCUGGGACGACU |
| | | CCUUCAACAACAAGGUGCUGGUGCUGGGCUCCGAGAACCAGAACAAGGGCAACCAGACCCCCUACGAGUACUUCAACGGCAAGGACAACUCCC |
| | | GGGAGUGGCAGGAGUUCAAGGCCCGGGUGGAGACCUCCCGGUUCCCCCGGUCCAAGAAGCAGCGGAUCCUGCUGCAGAAGUUCGACGAGGACG |
| | | GCUUCAAGGAGUGCAACCUGAACGACACCCGGUACGUGAACCGGUUCCUGUGCCAGUUCGUGGCCGACCACAUCCUGCUGACCGGCAAGGGCA |
| | | AGCGGCGGGUGUUCGCCUCCAACGGCCAGAUCACCAACCUGCUGCGGGGCUUCUGGGGCCUGCGGAAGGUGCGGGCCGAGAACGACCGGCACC |
| | | ACGCCCUGGACGCCGUGGUGGUGGCCUGCUCCACCGUGGCCAUGCAGCAGAAGAUCACCCGGUUCGUGCGGUACAAGGAGAUGAACGCCUUCG |
| | | ACGGCAAGACCAUCGACAAGGAGACCGGCAAGGUGCUGCACCAGAAGACCCACUUCCCCCAGCCCUGGGAGUUCUUCGCCCAGGAGGUGAUGA |
| | | UCCGGGUGUUCGGCAAGCCCGACGGCAAGCCCGAGUUCGAGGAGGCCGACACCCCCGAGAAGCUGCGGACCCUGCUGGCCGAGAAGCUGUCCU |
| | | CCCGGCCCGAGGCCGUGCACGAGUACGUGACCCCCCUGUUCGUGUCCCGGGCCCCCAACCGGAAGAUGUCCGGCGCCCACAAGGACACCCUGC |
| | | GGUCCGCCAAGCGGUUCGUGAAGCACAACGAGAAGAUCUCCGUGAAGCGGGUGUGGCUGACCGAGAUCAAGCUGGCCGACCUGGAGAACAUGG |
| | | UGAACUACAAGAACGGCCGGGAGAUCGAGCUGUACGAGGCCCUGAAGGCCCGGCUGGAGGCCUACGGCGGCAACGCCAAGCAGGCCUUCGACC |
| | | CCAAGGACAACCCCUUCUACAAGAAGGGCGGCCAGCUGGUGAAGGCCGUGCGGGUGGAGAAGACCCAGGAGUCCGGCGUGCUGCUGAACAAGA |
| | | AGAACGCCUACACCAUCGCCGACAACGGCGACAUGGUGCGGGUGGACGUGUUCUGCAAGGUGGACAAGAAGGGCAAGAACCAGUACUUCAUCG |
| | | UGCCCAUCUACGCCUGGCAGGUGGCCGAGAACAUCCUGCCCGACAUCGACUGCAAGGGCUACCGGAUCGACGACUCCUACACCUUCUGCUUCU |
| | | CCCUGCACAAGUACGACCUGAUCGCCUUCCAGAAGGACGAGAAGUCCAAGGUGGAGUUCGCCUACUACAUCAACUGCGACUCCUCCAACGGCC |
| | | GGUUCUACCUGGCCUGGCACGACAAGGGCUCCAAGGAGCAGCAGUUCCGGAUCUCCACCCAGAACCUGGUGCUGAUCCAGAAGUACCAGGUGA |
| | | ACGAGCUGGGCAAGGAGAUCCGGCCCUGCCGGCUGAAGAAGCGGCCCCCCGUGCGGUAGCUAGCACCAGCCUCAAGAACACCCGAAUGGAGUC |
| | | UCUAAGCUACAUAAUACCAACUUACACUUUACAAAAUGUUGUCCCCCAAAAUGUAGCCAUUCGUAUCUGCUCCUAAUAAAAAGAAAGUUUCUU |
| | | CACAUUCUCUCGAGAAAAAAAAAAAAUGGAAAAAAAAAAAACGGAAAAAAAAAAAAGGUAAAAAAAAAAAAUAUAAAAAAAAAAAACAUAAAA |
| | | AAAAAAAACGAAAAAAAAAAAACGUAAAAAAAAAAAACUCAAAAAAAAAAAGAUAAAAAAAAAAAACCUAAAAAAAAAAAAUGUAAAAAAAAA |
| | | AAAGGGAAAAAAAAAAAACGCAAAAAAAAAAAACACAAAAAAAAAAAAUGCAAAAAAAAAAAAUCGAAAAAAAAAAAAUCUAAAAAAAAAAAA |
| | | CGAAAAAAAAAAAACCCAAAAAAAAAAAAGACAAAAAAAAAAAAUAGAAAAAAAAAAAGUUAAAAAAAAAAAACUGAAAAAAAAAAAAUUUAA |
| | | AAAAAAAAAAUCUAG |
| mRNA | 826 | GGGAAGCUCAGAAUAAACGCUCAACUUUGGCCGGAUCUGCCACCAUGGACGGCUCCGGCGGCGGCUCCCCCAAGAAGAAGCGGAAGGUGGAGG |
| encoding | | ACAAGCGGCCCGCCGCCACCAAGAAGGCCGGCCAGGCCAAGAAGAAGAAGGGCGGCUCCGGCGGCGGCGCCGCCUUCAAGCCCAACCCCAUCA |
| Nme2 Cas9 | | ACUACAUCCUGGGCCUGGACAUCGGCAUCGCCUCCGUGGGCUGGGCCAUGGUGGAGAUCGACGAGGAGGAGAACCCCAUCCGGCUGAUCGACC |
| with Hibit tag | | UGGGCGUGCGGGUGUUCGAGCGGGCCGAGGUGCCCAAGACCGGCGACUCCCUGGCCAUGGCCCGGCGGCUGGCCCGGUCCGUGCGGCGGCUGA |
| | | CCCGGCGGCGGGCCCACCGGCUGCUGCGGGCCCGGCGGCUGCUGAAGCGGGAGGGCGUGCUGCAGGCCGCCGACUUCGACGAGAACGGCCUGA |
| | | UCAAGUCCCUGCCCAACACCCCCUGGCAGCUGCGGGCCGCCGCCCUGGACCGGAAGCUGACCCCCCUGGAGUGGUCCGCCGUGCUGCUGCACC |
| | | UGAUCAAGCACCGGGGCUACCUGUCCCAGCGGAAGAACGAGGGCGAGACCGCCGACAAGGAGCUGGGCGCCCUGCUGAAGGGCGUGGCCAACA |
| | | ACGCCCACGCCCUGCAGACCGGCGACUUCCGGACCCCCGCCGAGCUGGCCCUGAACAAGUUCGAGAAGGAGUCCGGCCACAUCCGGAACCAGC |
| | | GGGGCGACUACUCCCACACCUUCUCCCGGAAGGACCUGCAGGCCGAGCUGAUCCUGCUGUUCGAGAAGCAGAAGGAGUUCGGCAACCCCCACG |
| | | UGUCCGGCGGCCUGAAGGAGGGCAUCGAGACCCUGCUGAUGACCCAGCGGCCCGCCCUGUCCGGCGACGCCGUGCAGAAGAUGCUGGGCCACU |
| | | GCACCUUCGAGCCCGCCGAGCCCAAGGCCGCCAAGAACACCUACACCGCCGAGCGGUUCAUCUGGCUGACCAAGCUGAACAACCUGCGGAUCC |
| | | UGGAGCAGGGCUCCGAGCGGCCCCUGACCGACACCGAGCGGGCCACCCUGAUGGACGAGCCCUACCGGAAGUCCAAGCUGACCUACGCCCAGG |
| | | CCCGGAAGCUGCUGGGCCUGGAGGACACCGCCUUCUUCAAGGGCCUGCGGUACGGCAAGGACAACGCCGAGGCCUCCACCCUGAUGGAGAUGA |
| | | AGGCCUACCACGCCAUCUCCCGGGCCCUGGAGAAGGAGGGCCUGAAGGACAAGAAGUCCCCCCUGAACCUGUCCUCCGAGCUGCAGGACGAGA |
| | | UCGGCACCGCCUUCUCCCUGUUCAAGACCGACGAGGACAUCACCGGCCGGCUGAAGGACCGGGUGCAGCCCGAGAUCCUGGAGGCCCUGCUGA |
| | | AGCACAUCUCCUUCGACAAGUUCGUGCAGAUCUCCCUGAAGGCCCUGCGGCGGAUCGUGCCCCUGAUGGAGCAGGGCAAGCGGUACGACGAGG |
| | | CCUGCGCCGAGAUCUACGGCGACCACUACGGCAAGAAGAACACCGAGGAGAAGAUCUACCUGCCCCCCAUCCCCGCCGACGAGAUCCGGAACC |
| | | CCGUGGUGCUGCGGGCCCUGUCCCAGGCCCGGAAGGUGAUCAACGGCGUGGUGCGGCGGUACGGCUCCCCCGCCCGGAUCCACAUCGAGACCG |
| | | CCCGGGAGGUGGGCAAGUCCUUCAAGGACCGGAAGGAGAUCGAGAAGCGGCAGGAGGAGAACCGGAAGGACCGGGAGAAGGCCGCCGCCAAGU |
| | | UCCGGGAGUACUUCCCCAACUUCGUGGGCGAGCCCAAGUCCAAGGACAUCCUGAAGCUGCGGCUGUACGAGCAGCAGCACGGCAAGUGCCUGU |
| | | ACUCCGGCAAGGAGAUCAACCUGGUGCGGCUGAACGAGAAGGGCUACGUGGAGAUCGACCACGCCCUGCCCUUCUCCCGGACCUGGGACGACU |
| | | CCUUCAACAACAAGGUGCUGGUGCUGGGCUCCGAGAACCAGAACAAGGGCAACCAGACCCCCUACGAGUACUUCAACGGCAAGGACAACUCCC |
| | | GGGAGUGGCAGGAGUUCAAGGCCCGGGUGGAGACCUCCCGGUUCCCCCGGUCCAAGAAGCAGCGGAUCCUGCUGCAGAAGUUCGACGAGGACG |
| | | GCUUCAAGGAGUGCAACCUGAACGACACCCGGUACGUGAACCGGUUCCUGUGCCAGUUCGUGGCCGACCACAUCCUGCUGACCGGCAAGGGCA |
| | | AGCGGCGGGUGUUCGCCUCCAACGGCCAGAUCACCAACCUGCUGCGGGGCUUCUGGGGCCUGCGGAAGGUGCGGGCCGAGAACGACCGGCACC |
| | | ACGCCCUGGACGCCGUGGUGGUGGCCUGCUCCACCGUGGCCAUGCAGCAGAAGAUCACCCGGUUCGUGCGGUACAAGGAGAUGAACGCCUUCG |
| | | ACGGCAAGACCAUCGACAAGGAGACCGGCAAGGUGCUGCACCAGAAGACCCACUUCCCCCAGCCCUGGGAGUUCUUCGCCCAGGAGGUGAUGA |
| | | UCCGGGUGUUCGGCAAGCCCGACGGCAAGCCCGAGUUCGAGGAGGCCGACACCCCCGAGAAGCUGCGGACCCUGCUGGCCGAGAAGCUGUCCU |
| | | CCCGGCCCGAGGCCGUGCACGAGUACGUGACCCCCCUGUUCGUGUCCCGGGCCCCCAACCGGAAGAUGUCCGGCGCCCACAAGGACACCCUGC |
| | | GGUCCGCCAAGCGGUUCGUGAAGCACAACGAGAAGAUCUCCGUGAAGCGGGUGUGGCUGACCGAGAUCAAGCUGGCCGACCUGGAGAACAUGG |
| | | UGAACUACAAGAACGGCCGGGAGAUCGAGCUGUACGAGGCCCUGAAGGCCCGGCUGGAGGCCUACGGCGGCAACGCCAAGCAGGCCUUCGACC |
| | | CCAAGGACAACCCCUUCUACAAGAAGGGCGGCCAGCUGGUGAAGGCCGUGCGGGUGGAGAAGACCCAGGAGUCCGGCGUGCUGCUGAACAAGA |
| | | AGAACGCCUACACCAUCGCCGACAACGGCGACAUGGUGCGGGUGGACGUGUUCUGCAAGGUGGACAAGAAGGGCAAGAACCAGUACUUCAUCG |
| | | UGCCCAUCUACGCCUGGCAGGUGGCCGAGAACAUCCUGCCCGACAUCGACUGCAAGGGCUACCGGAUCGACGACUCCUACACCUUCUGCUUCU |
| | | CCCUGCACAAGUACGACCUGAUCGCCUUCCAGAAGGACGAGAAGUCCAAGGUGGAGUUCGCCUACUACAUCAACUGCGACUCCUCCAACGGCC |
| | | GGUUCUACCUGGCCUGGCACGACAAGGGCUCCAAGGAGCAGCAGUUCCGGAUCUCCACCCAGAACCUGGUGCUGAUCCAGAAGUACCAGGUGA |
| | | ACGAGCUGGGCAAGGAGAUCCGGCCCUGCCGGCUGAAGAAGCGGCCCCCCGUGCGGUCCGAGUCCGCCACCCCCGAGUCCGUGUCCGGCUGGC |
| | | GGCUGUUCAAGAAGAUCUCCUAGCUAGCACCAGCCUCAAGAACACCCGAAUGGAGUCUCUAAGCUACAUAAUACCAACUUACACUUUACAAAA |
| | | UGUUGUCCCCCAAAAUGUAGCCAUUCGUAUCUGCUCCUAAUAAAAAGAAAGUUUCUUCACAUUCUCUCGAGAAAAAAAAAAAAUGGAAAAAAA |
| | | AAAAACGGAAAAAAAAAAAAGGUAAAAAAAAAAAAUAUAAAAAAAAAAAACAUAAAAAAAAAAAACGAAAAAAAAAAAACGUAAAAAAAAAAA |
| | | ACUCAAAAAAAAAAAAGAUAAAAAAAAAAAACCUAAAAAAAAAAAAUGUAAAAAAAAAAAAGGGAAAAAAAAAAAACGCAAAAAAAAAAAACA |
| | | CAAAAAAAAAAAAUGCAAAAAAAAAAAAUCGAAAAAAAAAAAAUCUAAAAAAAAAAAACGAAAAAAAAAAAACCCAAAAAAAAAAAAGACAAA |
| | | AAAAAAAAAUAGAAAAAAAAAAAAGUUAAAAAAAAAAAACUGAAAAAAAAAAAAUUUAAAAAAAAAAAAUCUAG |
| Wild-type | 899 | GUUGUAGCUCCCUUUCUCAUUUCGGAAACGAAAUGAGAACCGUUGCUACAAUAAGGCCGUCUGAAAAGAUGUGCCGCAACGCUCUGCCCCUUA |
| NmeCas9 | | AAGCUUCUGCUUUAAGGGGCAUCGUUUA |
| guide RNA-conserved portion | | |
| only | | |
| Wild-type | 900 | NNNNNNNNNNNNNNNNNNNNNNNNGUUGUAGCUCCCUUUCUCAUUUCGGAAACGAAAUGAGAACCGUUGCUACAAUAAGGCCGUCUGAAAAGA |
| NmeCas9 guide RNA | | UGUGCCGCAACGCUCUGCCCCUUAAAGCUUCUGCUUUAAGGGGCAUCGUUUA |
| Shortened/unmodified | 901 | (N)₂₀₋₂₅ GUUGUAGCUCCCUGAAACCGUUGCUACAAUAAGGCCGUCGAAAGAUGUGCCGCAACGCUCUGCCUUCUGGCAUCGUU |
| NmeCas9 guide RNA motif | | |
| Shortened/unmodified | 902 | (N)₂₀₋₂₅ GUUGUAGCUCCCUGAAACCGUUGCUACAAUAAGGCCGUCGAAAGAUGUGCCGCAACGCUCUGCCUUCUGGCAUCGUUUAUU |
| NmeCas9 guide RNA motif | | |
| Shortened/unmodified | 903 | (N)₂₀₋₂₅ GUUGUAGCUCCCUGGAAACCCGUUGCUACAAUAAGGCCGUCGAAAGAUGUGCCGCAACGCUCUGCCUUCUGGCAUCGUUUAUU |
| NmeCas9 guide RNA motif | | |
| Shortened/modified | 904 | mN*mNNNNNNNNmNNNmNNNNNNNNNNNNmGUUGmUmAmGmCUCCCmUmGmAmAmAmCmCGUUmGmCUAmCAAU*AAGmGmCCmGmUmCmGmA |
| NmeCas9 guide RNA motif | | mAmAmGmAmUGUGCmCGCmAmAmCmGCUCUmGmCCmUmUmCmUGmGCmAmUC*mG*mU*mU |
| (101-mer) | | |
| Shortened/modified | 905 | (N)₂₀₋ |
| NmeCas9 guide RNA motif | | 25GUUGmUmAmGmCUCCCmUmGmAmAmAmCmCGUUmGmCUAmCAAU*AAGmGmCCmGmUmCmGmAmAmAmGmAmUGUGCmCGCmAmAmCmGCU |
| | | CUmGmCCmUmUmCmUGmGCmAmUC*mG*mU*mU |
| Shortened/modified | 906 | GUUGmUmAmGmCUCCCmUmGmAmAmAmCmCGUUmGmCUAmCAAU*AAGmGmCCmGmUmCmGmAmAmAmGmAmUGUGCmCGCmAmAmCmGCUCU |
| NmeCas9 guide RNA motif | | mGmCCmUmUmCmUGmGCmAmUC*mG*mU*mU |
| Shortened/modified | 907 | mN*mN*mN*mNmNNNmNmNNmNNmNNNNNmNNNNmNNNmGUUGmUmAmGmCUCCCmUmGmAmAmAmCmCGUUmGmCUAmCAAU*AAGmGmCCm |
| NmeCas9 guide RNA motif | | GmUmCmGmAmAmAmGmAmUGUGCmCGmCAAmCGCUCUmGmCCmUmUmCmUGGCAUCG*mU*mU |
| (101-mer) | | |
| Shortened/modified | 908 | (N)₂₀₋₂₅ |
| NmeCas9 guide RNA motif | | GUUGmUmAmGmCUCCCmUmGmAmAmAmCmCGUUmGmCUAmCAAU*AAGmGmCCmGmUmCmGmAmAmAmGmAmUGUGCmCGmCAAmCGCUCU |
| | | mGmCCmUmUmCmUGGCAUCG*mU*mU |
| Shortened/modified | 909 | GUUGmUmAmGmCUCCCmUmGmAmAmAmCmCGUUmGmCUAmCAAU*AAGmGmCCmGmUmCmGmAmAmAmGmAmUGUGCmCGmCAAmCGCUCUmG |
| NmeCas9 guide RNA motif | | mCCmUmUmCmUGGCAUCG*mU*mU |
| Shortened/unmodified | 910 | (N)₂₀₋₂₅GUUGUAGCUCCCUUCGAAAGACCGUUGCUACAAUAAGGCCGUCGAAAGAUGUGCCGCAACGCUCUGCCUUCUGGCAUCGUU |
| NmeCas9 guide RNA motif | | |
| Shortened/modified | 911 | mN*mN*mN*mNmNNNmNmNNmNNmNNNNNmNNNNmNNNmGUUGmUmAmGmCUCCCmUmUmCmGmAmAmAmGmAmCmCGUUmGmCUAmCAAU*A |
| NmeCas9 guide RNA motif | | AGmGmCCmGmUmCmGmAmAmAmGmAmUGUGCmCGmCAAmCGCUCUmGmCCmUmUmCmUGGCAUCG*mU*mU |
| (105-mer) | | |
| Exemplary | 912 | LAAKRSRTT |
| NLS1 | | |
| Exemplary | 913 | QAAKRSRTT |
| NLS2 | | |
| Exemplary | 914 | PAPAKRERTT |
| NLS3 | | |
| Exemplary | 915 | QAAKRPRTT |
| NLS4 | | |
| Exemplary | 916 | RAAKRPRTT |
| NLS5 | | |
| Exemplary | 917 | AAAKRSWSMAA |
| NLS6 | | |
| Exemplary | 918 | AAAKRVWSMAF |
| NLS7 | | |
| Exemplary | 919 | AAAKRSWSMAF |
| NLS8 | | |
| Exemplary | 920 | AAAKRKYFAA |
| NLS9 | | |
| Exemplary | 921 | RAAKRKAFAA |
| NLS10 | | |
| Exemplary | 922 | RAAKRKYFAV |
| NLS11 | | |
| Altern- | 923 | PKKKRRV |
| ative | | |
| SV40 NLS | | |
| Nucleo- | 924 | KRPAATKKAGQAKKKK |
| plasmin | | |
| MLS | | |
| exemplary | 930 | SGSETPGTSESATPES |
| XTEN | | |
| exemplary | 931 | SGSETPGTSESA |
| XTEN | | |
| exemplary | 932 | SGSETPGTSESATPEGGSGGS |
| XTEN | | |
| amino acid | 933 | GGS |
| sequence | | |
| for | | |
| exemplary | | |
| linker | | |
| amino acid sequence | 934 | GGGGS |
| for exemplary linker | | |
| amino acid sequence | 935 | EAAAK |
| for exemplary linker | | |
| amino acid sequence | 936 | SEGSA |
| for exemplary linker | | |
| amino acid sequence | 937 | SEGSAGTST |
| for exemplary linker | | |
| amino acid sequence | 938 | GGGGSGGGGS |
| for exemplary linker | | |
| amino acid sequence | 939 | GGGGSEAAAK |
| for exemplary linker | | |
| amino acid sequence | 940 | EAAAKGGGGS |
| for exemplary linker | | |
| amino acid sequence | 941 | EAAAKEAAAK |
| for exemplary linker | | |
| amino acid sequence | 942 | SEGSAGTSTESEGSA |
| for exemplary linker | | |
| amino acid sequence | 943 | GGGGSGGGGSGGGGS |
| for exemplary linker | | |
| amino acid sequence | 944 | GGGGSGGGGSEAAAK |
| for exemplary linker | | |
| amino acid sequence | 945 | GGGGSEAAAKGGGGS |
| for exemplary linker | | |
| amino acid sequence | 946 | EAAAKGGGGSEAAAK |
| for exemplary linker | | |
| amino acid sequence | 947 | EAAAKEAAAKGGGGS |
| for exemplary linker | | |
| amino acid sequence | 948 | SEGSAGTSTESEGSAGTSTE |
| for exemplary linker | | |
| amino acid sequence | 949 | GGGGSGGGGSGGGGSEAAAK |
| for exemplary linker | | |
| amino acid sequence | 950 | GGGGSGGGGSEAAAKGGGGS |
| for exemplary linker | | |
| amino acid sequence | 951 | GGGGSEAAAKGGGGSEAAAK |
| for exemplary linker | | |
| amino acid sequence | 952 | GGGGSEAAAKEAAAKGGGGS |
| for exemplary linker | | |
| amino acid sequence | 953 | GGGGSEAAAKEAAAKEAAAK |
| for exemplary linker | | |
| amino acid sequence | 954 | EAAAKGGGGSGGGGSGGGGS |
| for exemplary linker | | |
| amino acid sequence | 955 | EAAAKGGGGSGGGGSEAAAK |
| for exemplary linker | | |
| amino acid sequence | 956 | EAAAKGGGGSEAAAKGGGGS |
| for exemplary linker | | |
| amino acid sequence | 957 | EAAAKGGGGSEAAAKEAAAK |
| for exemplary linker | | |
| amino acid sequence | 958 | EAAAKEAAAKGGGGSGGGGS |
| for exemplary linker | | |
| amino acid sequence | 959 | EAAAKEAAAKGGGGSEAAAK |
| for exemplary linker | | |
| amino acid sequence | 960 | EAAAKEAAAKEAAAKGGGGS |
| for exemplary linker | | |
| amino acid sequence | 961 | SEGSAGTSTESEGSAGTSTESEGSA |
| for exemplary linker | | |
| amino acid sequence | 962 | GGGGSGGGGSGGGGSGGGGSGGGGS |
| for exemplary linker | | |
| amino acid sequence | 963 | GGGGGGGGSGGGGSGGGGSEAAAK |
| for exemplary linker | | |
| amino acid sequence | 964 | GGGGSGGGGSGGGGSEAAAKGGGGS |
| for exemplary linker | | |
| amino acid sequence | 965 | GGGGSGGGGSGGGGSEAAAKEAAAK |
| for exemplary linker | | |
| amino acid sequence | 966 | GGGGSGGGGSEAAAKGGGGSGGGGS |
| for exemplary linker | | |
| amino acid sequence | 967 | GGGGSGGGGSEAAAKGGGGSEAAAK |
| for exemplary linker | | |
| amino acid sequence | 968 | GGGGSGGGGSEAAAKEAAAKGGGGS |
| for exemplary linker | | |
| amino acid sequence | 969 | GGGGSGGGGSEAAAKEAAAKEAAAK |
| for exemplary linker | | |
| amino acid sequence | 970 | GGGGSEAAAKGGGGSGGGGSGGGGS |
| for exemplary linker | | |
| amino acid sequence | 971 | GGGGSEAAAKGGGGSGGGGSEAAAK |
| for exemplary linker | | |
| amino acid sequence | 972 | GGGGSEAAAKGGGGSEAAAKGGGGS |
| for exemplary linker | | |
| amino acid sequence | 973 | GGGGSEAAAKGGGGSEAAAKEAAAK |
| for exemplary linker | | |
| amino acid sequence | 974 | GGGGSEAAAKEAAAKGGGGSGGGGS |
| for exemplary linker | | |
| amino acid sequence | 975 | GGGGSEAAAKEAAAKEAAAKGGGGS |
| for exemplary linker | | |
| amino acid sequence | 976 | GGGGSEAAAKEAAAKEAAAKEAAAK |
| for exemplary linker | | |
| amino acid sequence | 977 | EAAAKGGGGSGGGGSGGGGSGGGGS |
| for exemplary linker | | |
| amino acid sequence | 978 | EAAAKGGGGSGGGGSGGGGSEAAAK |
| for exemplary linker | | |
| amino acid sequence | 979 | EAAAKGGGGSGGGGSEAAAKGGGGS |
| for exemplary linker | | |
| amino acid sequence | 980 | EAAAKGGGGSGGGGSEAAAKEAAAK |
| for exemplary linker | | |
| amino acid sequence | 981 | EAAAKGGGGSEAAAKGGGGSGGGGS |
| for exemplary linker | | |
| amino acid sequence | 982 | EAAAKGGGGSEAAAKGGGGSEAAAK |
| for exemplary linker | | |
| amino acid sequence | 983 | EAAAKGGGGSEAAAKEAAAKGGGGS |
| for exemplary linker | | |
| amino acid sequence | 984 | EAAAKGGGGSEAAAKEAAAKEAAAK |
| for exemplary linker | | |
| amino acid sequence | 985 | EAAAKEAAAKGGGGSEAAAKGGGGS |
| for exemplary linker | | |
| amino acid sequence | 986 | EAAAKEAAAKGGGGSEAAAKEAAAK |
| for exemplary linker | | |
| amino acid sequence | 987 | EAAAKEAAAKEAAAKGGGGSEAAAK |
| for exemplary linker | | |
| amino acid sequence | 988 | EAAAKEAAAKEAAAKEAAAKGGGGS |
| for exemplary linker | | |
| amino acid sequence | 989 | EAAAKEAAAKEAAAKEAAAKEAAAK |
| for exemplary linker | | |
| amino acid sequence | 990 | GTKDSTKDIPETPSKD |
| for exemplary linker | | |
| amino acid sequence | 991 | GRDVRQPEVKEEKPES |
| for exemplary linker | | |
| amino acid sequence | 992 | EGKSSGSGSESKSTAG |
| for exemplary linker | | |
| amino acid sequence | 993 | TPGSPAGSPTSTEEGT |
| for exemplary linker | | |
| amino acid sequence | 994 | GSEPATSGSETPGTST |
| for exemplary linker | | |
| Exemplary modified | 995 | mN*mN*mN*mNmNNNmNmNNmNNmNNNNNmNNNNmNNNmGUUGmUmAmGmCUCCCmUmGmAmAmAmCmCGUUmGmCUAmCAAUAAGmGmCCmG |
| Nme guide sgRNA | | mUmCmGmAmAmAmGmAmUGUGCmCGmCAAmCGCUCUmGmCCmUmUmCmUGGCAUCG*mU*mU |
| Exemplary modified | 996 | mN*mN*mN*mNmNNNmNmNNmNNmNNNNNmNNNNmNNNmGUUGmUmAmGmCUCCCmUmGmAmAmAmCmCGUUmGmCUAmCAAU*AAGmGmCCm |
| Nme guide sgRNA | | GmUmCmGmAmAmAmGmAmUGUGCmCGmCAAmCGmCmUmCmUmGmCCmUmUmCmUGGCAUCG*mU*mU |
| Exemplary modified | 997 | mN*mN*mN*mNmNNNmNmNNmNNmNNNNNmNNNNmNNNmGUUGmUmAmGmCUCCCmUmGmAmAmAmCmCGUUmGmCUAmCAAUAAGmGmCCmG |
| Nme guide sgRNA | | mUmCmGmAmAmAmGmAmUGUGCmCGmCAAmCGmCmUmCmUmGmCCmUmUmCmUGGCAUCG*mU*mU |

*The guide sequence disclosed in this Table may be unmodified, modified with the exemplary modification pattern shown in the Table, or modified with a different
modification pattern disclosed herein or available in the art.

TABLE 7A
VI. Additional Exemplary Guide RNAs

| | | | | Genomic |
|---|---|---|---|---|
| | | Guide | | Coordinates |
| Guide ID | Target | Sequence | Exemplary Guide RNA Modified Sequence | (hg38) |
| G034202 | HLA-A | GCUCUAUC | mG*mC*mU*mCmUAUmCmCAmCGmGCGCCmCGCGmGCUmGUUGmUmAmGmCU | chr6: 29942891- |
| | | CACGGCGC | CCCmUmGmAmAmAmCmCGUUmGmCUAmCAAUAAGmGmCCmGmUmCmGmAmA | 29942915 |
| | | CCGCGGCU | mAmGmAmUGUGCmCGmCAAmCGCUCUmGmCCmUmUmCmUGGCAUCG*mU*mU | |
| | | (SEQ ID | (SEQ ID NO: 3001) | |
| | | NO: 66) | | |
| G034617 | HLA-A | CACUCACC | mC*mA*mC*mUmCACmCmCGmCCmCAGGUmCUGGmGUCmGUUGmUmAmGmCU | chr6: 29942609- |
| | | CGCCCAGG | CCCmUmGmAmAmAmCmCGUUmGmCUAmCAAUAAGmGmCCmGmUmCmGmAmA | 29942633 |
| | | UCUGGGUC | mAmGmAmUGUGCmCGmCAAmCGCUCUmGmCCmUmUmCmUGGCAUCG*mU*mU | |
| | | (SEQ ID | (SEQ ID NO: 3002) | |
| | | NO: 61) | | |
| G034982 | TRAC | AAAACCUG | mA*mA*mA*mAmCCUmGmUCmAGmUGAUUmGGGUmUCCmGUUGmUmAmGmCU | chr14: 22550574- |
| | | UCAGUGA | CCCmUmGmAmAmAmCmCGUUmGmCUAmCAAUAAGmGmCCmGmUmCmGmAmA | 22550598 |
| | | UUGGGUU | mAmGmAmUGUGCmCGmCAAmCGCUCUmGmCCmUmUmCmUGGCAUCG*mU*mU | |
| | | CC | (SEQ ID NO: 3003) | |
| | | (SEQ ID | | |
| | | NO: 111) | | |
| G034981 | TRAC | UUAGGUU | mU*mU*mA*mGmGUUmCmGUmAUmCUGUAmAAACmCAAmGUUGmUmAmGmCU | chr14: 22550544- |
| | | CGUAUCUG | CCCmUmGmAmAmAmCmCGUUmGmCUAmCAAUAAGmGmCCmGmUmCmGmAmA | 22550568 |
| | | UAAAACCA | mAmGmAmUGUGCmCGmCAAmCGCUCUmGmCCmUmUmCmUGGCAUCG*mU*mU | |
| | | A (SEQ ID | (SEQ ID NO: 3004) | |
| | | NO: 107) | | |
| G034618 | TRBC1 | GUGUCCUA | mG*mU*mG*mUmCCUmAmCCmAGmCAAGGmGGUCmCUGmGUUGmUmAmGmCU | chr7: 142792690- |
| | | CCAGCAAG | CCCmUmGmAmAmAmCmCGUUmGmCUAmCAAUAAGmGmCCmGmUmCmGmAmA | 142792714 |
| | | GGGUCCUG | mAmGmAmUGUGCmCGmCAAmCGCUCUmGmCCmUmUmCmUGGCAUCG*mU*mU | |
| | | (SEQ ID | (SEQ ID NO: 3005) | |
| | | NO: 215) | | |
| G034201 | CIITA | UCAAAGU | mU*mC*mA*mAmAGUmAmCCmCUmACAGGmAGGAmCCAmGUUGmUmAmGmCU | chr16: 10907504- |
| | | ACCCUACA | CCCmUmGmAmAmAmCmCGUUmGmCUAmCAAUAAGmGmCCmGmUmCmGmAmA | 10907528 |
| | | GGAGGACC | mAmGmAmUGUGCmCGmCAAmCGCUCUmGmCCmUmUmCmUGGCAUCG*mU*mU | |
| | | A (SEQ ID | (SEQ ID NO: 3006) | |
| | | NO: 301) | | |
| G034619 | CIITA | AGCUGCCG | mA*mG*mC*mUmGCCmGmUUmCUmGCCCAmGUCCmGGGmGUUGmUmAmGmCU | chr16: 10906643- |
| | | UUCUGCCC | CCCmUmGmAmAmAmCmCGUUmGmCUAmCAAUAAGmGmCCmGmUmCmGmAmA | 10906667 |
| | | AGUCCGGG | mAmGmAmUGUGCmCGmCAAmCGCUCUmGmCCmUmUmCmUGGCAUCG*mU*mU | |
| | | (SEQ ID | (SEQ ID NO: 3007) | |
| | | NO: 422) | | |
| G032794 | HLA-B | UCUGGGA | mU*mC*mU*mGmGGAmAmAGmGAmGGGGAmAGAUmGAGmGUUGmUmAmGmCU | chr6: 31355222- |
| | | AAGGAGG | CCCmUmGmAmAmAmCmCGUUmGmCUAmCAAU*AAGmGmCCmGmUmCmGmAm | 31355246 |
| | | GGAAGAU | AmAmGmAmUGUGCmCGmCAAmCGCUCUmGmCCmUmUmCmUGGCAUCG*mU*m | |
| | | GAG (SEQ | U (SEQ ID NO: 2828) | |
| | | ID NO: | | |
| | | 828) | | |
| G034208 | HLA-B | UCUGGGA | mU*mC*mU*mGmGGAmAmAGmGAmGGGGAmAGAUmGAGmGUUGmUmAmGmCU | chr6: 31355222- |
| | | AAGGAGG | CCCmUmGmAmAmAmCmCGUUmGmCUAmCAAUAAGmGmCCmGmUmCmGmAmA | 31355246 |
| | | GGAAGAU | mAmGmAmUGUGCmCGmCAAmCGCUCUmGmCCmUmUmCmUGGCAUCG*mU*mU | |
| | | GAG (SEQ | (SEQ ID NO: 3008) | |
| | | ID NO: | | |
| | | 828) | | |
| G032795 | HLA-B | CUCUGGGA | mC*mU*mC*mUmGGGmAmAAmGGmAGGGGmAAGAmUGAmGUUGmUmAmGmCU | chr6: 31355221- |
| | | AAGGAGG | CCCmUmGmAmAmAmCmCGUUmGmCUAmCAAU*AAGmGmCCmGmUmCmGmAm | 31355245 |
| | | GGAAGAU | AmAmGmAmUGUGCmCGmCAAmCGCUCUmGmCCmUmUmCmUGGCAUCG*mU*m | |
| | | GA (SEQ | U (SEQ ID NO: 2829) | |
| | | ID NO: | | |
| | | 829) | | |
| G034209 | HLA-B | CUCUGGGA | mC*mU*mC*mUmGGGmAmAAmGGmAGGGGmAAGAmUGAmGUUGmUmAmGmCU | chr6: 31355221- |
| | | AAGGAGG | CCCmUmGmAmAmAmCmCGUUmGmCUAmCAAUAAGmGmCCmGmUmCmGmAmA | 31355245 |
| | | GGAAGAU | mAmGmAmUGUGCmCGmCAAmCGCUCUmGmCCmUmUmCmUGGCAUCG*mU*mU | |
| | | GA (SEQ | (SEQ ID NO: 3009) | |
| | | ID NO: | | |
| | | 829) | | |
| G032806 | HLA-B | UCCCAGAG | mU*mC*mC*mCmAGAmGmCCmGUmCUUCCmCAGUmCCAmGUUGmUmAmGmCU | chr6: 31355205- |
| | | CCGUCUUC | CCCmUmGmAmAmAmCmCGUUmGmCUAmCAAU*AAGmGmCCmGmUmCmGmAm | 31355229 |
| | | CCAGUCCA | AmAmGmAmUGUGCmCGmCAAmCGCUCUmGmCCmUmUmCmUGGCAUCG*mU*m | |
| | | (SEQ ID | U (SEQ ID NO: 2830) | |
| | | NO: 830) | | |
| G034211 | HLA-B | UCCCAGAG | mU*mC*mC*mCmAGAmGmCCmGUmCUUCCmCAGUmCCAmGUUGmUmAmGmCU | chr6: 31355205- |
| | | CCGUCUUC | CCCmUmGmAmAmAmCmCGUUmGmCUAmCAAUAAGmGmCCmGmUmCmGmAmA | 31355229 |
| | | CCAGUCCA | mAmGmAmUGUGCmCGmCAAmCGCUCUmGmCCmUmUmCmUGGCAUCG*mU*mU | |
| | | (SEQ ID | (SEQ ID NO: 3010) | |
| | | NO: 830) | | |
| G021557 | VEGFA | GCAUGGGC | mG*mC*mA*mUmGGGmCmAGmGGmGCUGGmGGUGmCACmGUUGmUmAmGmCU | chr6: 43774288- |
| | | AGGGGCU | CCCmUmGmAmAmAmCmCGUUmGmCUAmCAAU*AAGmGmCCmGmUmCmGmAm | 43774312 |
| | | GGGGUGC | AmAmGmAmUGUGCmCGmCAAmCGCUCUmGmCCmUmUmCmUGGCAUCG*mU*m | |
| | | AC (SEQ | U (SEQ ID NO: 2831) | |
| | | ID NO: | | |
| | | 831) | | |
| G021558 | VEGFA | GAAUGGC | mG*mA*mA*mUmGGCmAmGGmCGmGAGGUmUGUAmCUGmGUUGmUmAmGmCU | chr6: 43780852- |
| | | AGGCGGA | CCCmUmGmAmAmAmCmCGUUmGmCUAmCAAU*AAGmGmCCmGmUmCmGmAm | 43780876 |
| | | GGUUGUA | AmAmGmAmUGUGCmCGmCAAmCGCUCUmGmCCmUmUmCmUGGCAUCG*mU*m | |
| | | CUG (SEQ | U (SEQ ID NO: 2832) | |
| | | ID NO: | | |
| | | 832) | | |
| G021567 | VEGFA | GUGAGCA | mG*mU*mG*mAmGCAmGmGCmACmCUGUGmCCAAmCAUmGUUGmUmAmGmCU | chr6: 43781113- |
| | | GGCACCUG | CCCmUmGmAmAmAmCmCGUUmGmCUAmCAAU*AAGmGmCCmGmUmCmGmAm | 43781137 |
| | | UGCCAACA | AmAmGmAmUGUGCmCGmCAAmCGCUCUmGmCCmUmUmCmUGGCAUCG*mU*m | |
| | | U (SEQ ID | U (SEQ ID NO: 2833) | |
| | | NO: 833) | | |

TABLE 7B
VII. Exemplary Modified Conserved Region Nme Guide RNA Motifs

| | | SEQ |
|---|---|---|
| | | ID |
| #mer | Modified nucleotide sequence of conserved portion | NO |
| 101 | mGUUGmUmAmGmCUCCCmUmGmAmAmAmCmCGUUmGmCUAmCAA | 1081 |
| | U*AAGmGmCCmGmUmCmGmAmAmAmGmAmUGUGCmCGCmAmAmC | |
| | mGCUCUmGmCCmUmUmCmUGmGCmAmUC*mG*mU*mU | |
| 101 | mGUUGmUmAmGmCUCCCmUmGmAmAmAmCmCGUUmGmCUAmCAA | 1082 |
| | U*AAGmGmCCmGmUmCmGmAmAmAmGmAmUGUGCmCGmCAAmCG | |
| | CUCUmGmCCmUmUmCmUGGCAUCG*mU*mU | |
| 101 | mGUUGmUmAmGmCUCCCmUmGmAmAmAmCmCGUUmGmCUAmCAA | 1083 |
| | UAAGmGmCCmGmUmCmGmAmAmAmGmAmUGUGCmCGmCAAmCGC | |
| | UCUmGmCCmUmUmCmUGGCAUCG*mU*mU | |
| 101 | mGUUGmUmAmGmCUCCCmUmGmAmAmAmCmCGUUmGmCUAmCAA | 1084 |
| | U*AAGmGmCCmGmUmCmGmAmAmAmGmAmUGUGCmCGmCAAmCG | |
| | mCmUmCmUmGmCCmUmUmCmUGGCAUCG*mU*mU | |
| 101 | mGUUGmUmAmGmCUCCCmUmGmAmAmAmCmCGUUmGmCUAmCAA | 1085 |
| | UAAGmGmCCmGmUmCmGmAmAmAmGmAmUGUGCmCGmCAAmCGm | |
| | CmUmCmUmGmCCmUmUmCmUGGCAUCG*mU*mU | |
| 105 | mGUUGmUmAmGmCUCCCmUmUmCmGmAmAmAmGmAmCmCGUUmG | 1086 |
| | mCUAmCAAU*AAGmGmCCmGmUmCmGmAmAmAmGmAmUGUGCmC | |
| | GmCAAmCGCUCUmGmCCmUmUmCmUGGCAUCG*mU*mU | |
| 105 | mGUUGmUmAmGmCUCCCmUmUmCmGmAmAmAmGmAmCmCGUUmG | 1087 |
| | mCUAmCAAUAAGmGmCCmGmUmCmGmAmAmAmGmAmUGUGCmCG | |
| | mCAAmCGCUCUmGmCCmUmUmCmUGGCAUCG*mU*mU | |
| 105 | mGUUGmUmAmGmCUCCCmUmUmCmGmAmAmAmGmAmCmCGUUmG | 1088 |
| | mCUAmCAAU*AAGmGmCCmGmUmCmGmAmAmAmGmAmUGUGCmC | |
| | GmCAAmCGmCmUmCmUmGmCCmUmUmCmUGGCAUCG*mU*mU | |
| 105 | mGUUGmUmAmGmCUCCCmUmUmCmGmAmAmAmGmAmCmCGUUmG | 1089 |
| | mCUAmCAAUAAGmGmCCmGmUmCmGmAmAmAmGmAmUGUGCmCG | |
| | mCAAmCGmCmUmCmUmGmCCmUmUmCmUGGCAUCG*mU*mU | |

### EXAMPLES

The following examples are provided to illustrate certain disclosed embodiments and are not to be construed as limiting the scope of this disclosure in any way.

### Example 1: General Methods

Generally, unless otherwise indicated, guide RNAs used throughout the Examples identified as “GXXXXXX” refer to 89-nt or 101-nt modified sgRNA format such as those shown in the Tables provided herein.

### 1.1 Next-Generation Sequencing (“NGS”) and Analysis for On-Target Cleavage Efficiency.

Genomic DNA was extracted using QuickExtract™ DNA Extraction Solution (Lucigen, Cat. No. QE09050) according to manufacturer's protocol.

To quantitatively determine the efficiency of editing at the target location in the genome, deep sequencing was utilized to identify the presence of insertions, deletions, and substitution introduced by gene editing. PCR primers were designed around the target site within the gene of interest (e.g., HLA-A) and the genomic area of interest was amplified. Primer sequence design was done as is standard in the field.

Additional PCR was performed according to the manufacturer's protocols (Illumina) to add chemistry for sequencing. The amplicons were sequenced on an Illumina MiSeq or NextSeq instrument. The reads were aligned to the human reference genome (e.g., hg38) after eliminating those having low quality scores. Reads that overlapped the target region of interest were re-aligned to the local genome sequence to improve the alignment. Then the number of wild type reads versus the number of reads which contain C-to-T mutations, C-to-A/G mutations or indels was calculated. Insertions and deletions were scored in a 20 bp region centered on the predicted Cas9 cleavage site. Indel percentage is defined as the total number of sequencing reads with one or more base inserted or deleted within the 20 bp scoring region divided by the total number of sequencing reads, including wild type. C-to-T mutations or C-to-A/G mutations were scored in a 40 bp region including 10 bp upstream and 10 bp downstream of the 20 bp sgRNA target sequence. The C-to-T editing percentage is defined as the total number of sequencing reads with either one or more C-to-T mutations within the 40 bp region divided by the total number of sequencing reads, including wild type. The percentage of C-to-A/G mutations are calculated similarly.

1.2 In Vitro Transcription (“IVT”) of mRNA

Capped and polyadenylated mRNA containing N1-methyl pseudo-U was generated by in vitro transcription using a linearized plasmid DNA template and T7 RNA polymerase. Plasmid DNA containing a T7 promoter, a sequence for transcription, and a polyadenylation sequence was linearized by incubating at 37° C. for 2 hours with XbaI with the following conditions: 200 ng/μL plasmid, 2 U/μL XbaI (NEB), and 1× reaction buffer. The XbaI was inactivated by heating the reaction at 65° C. for 20 min. The linearized plasmid was purified from enzyme and buffer salts. The IVT reaction to generate modified mRNA was performed by incubating at 37° C. for 1.5-4 hours in the following conditions: 50 ng/μL linearized plasmid; 2-5 mM each of GTP, ATP, CTP, and N1-methyl pseudo-UTP (Trilink); 10-25 mM ARCA (Trilink); 5 U/μL T7 RNA polymerase (NEB); 1 U/μL Murine RNase inhibitor (NEB); 0.004 U/μL Inorganic E. coli pyrophosphatase (NEB); and 1× reaction buffer. TURBO DNase (ThermoFisher) was added to a final concentration of 0.01 U/μL, and the reaction was incubated for an additional 30 minutes to remove the DNA template. The mRNA was purified using a MegaClear Transcription Clean-up kit (ThermoFisher) or a RNeasy Maxi kit (Qiagen) per the manufacturers' protocols. Alternatively, the mRNA was purified through a precipitation protocol, which in some cases was followed by HPLC-based purification. Briefly, after the DNase digestion, mRNA is purified using LiCl precipitation, ammonium acetate precipitation and sodium acetate precipitation. For HPLC purified mRNA, after the LiCl precipitation and reconstitution, the mRNA was purified by RP-IP HPLC (see, e.g., Kariko, et al. Nucleic Acids Research, 2011, Vol. 39, No. 21 e142). The fractions chosen for pooling were combined and desalted by sodium acetate/ethanol precipitation as described above. In a further alternative method, mRNA was purified with a LiCl precipitation method followed by further purification by tangential flow filtration. RNA concentrations were determined by measuring the light absorbance at 260 nm (Nanodrop), and transcripts were analyzed by capillary electrophoresis by Bioanlayzer (Agilent).

Streptococcus pyogenes (“Spy”) Cas9 mRNA was generated from plasmid DNA encoding an open reading frame according to SEQ ID NOs: 812-817 (see sequences in Table 7). When SEQ ID NOs: 812-817 are referred to below with respect to RNAs, it is understood that Ts should be replaced with Us (which were N1-methyl pseudouridines as described above). Messenger RNAs used in the Examples include a 5′ cap and a 3′ polyadenylation region, e.g., up to 100 nts, and are identified by the SEQ ID NOs: 812-817 in Table 7.

### 1.3 T Cell Preparation

Healthy human donor apheresis was obtained commercially (Hemacare), and cells were washed and resuspended in in CliniMACS® PBS/EDTA buffer (Miltenyi Biotec Cat. 130-070-525) and processed in a MultiMACS™ Cell 24 Separator Plus device (Miltenyi Biotec). T cells were isolated via positive selection using a Straight from Leukopak® CD4/CD8 MicroBead kit, human (Miltenyi Biotec Cat. 130-122-352). T cells were aliquoted and cryopreserved for future use in Cryostor® CS10 (StemCell Technologies Cat. 07930). Upon thaw, T cells were plated at a density of 1.0×10⁶ cells/mL in T cell growth media (TCGM) composed of CTS OpTmizer T Cell Expansion SFM and T Cell Expansion Supplement (ThermoFisher Cat. A1048501) containing 2.5% or 5% human AB serum (GeminiBio, Cat. 100-512), 1× Penicillin-Streptomycin, 1× Glutamax, 10 mM HEPES, further supplemented with 200 U/mL recombinant human interleukin-2 (Peprotech, Cat. 200-02), 5 ng/ml recombinant human interleukin 7 (Peprotech, Cat. 200-07), and 5 ng/ml recombinant human interleukin 15 (Peprotech, Cat. 200-15). T cells were rested in the T cell growth media for 24 hours at which time they were activated with TransAct™ (1:100 dilution, Miltenyi Biotec, Cat. 130-111-160). T cells were activated for 48 hours prior to electroporation.

### 1.4 RNA Electroporation

Guide RNAs targeting a specific gene were removed from their storage plates and denatured for 2 minutes at 95° C. before incubating at room temperature for 5 minutes. Forty-eight hours post T cell activation, T cells were harvested, centrifuged at 500 g for 5 minutes, and resuspended at a concentration of 12.5×10⁶ T cells/mL in P3 electroporation buffer (Lonza Catalog #V4SP-3960). BC22n electroporation mix was prepared with 1×10⁵ T cells, 20 ng/μL of BC22n mRNAs, 20 ng/μL of UGI mRNA and 20 pmols of sgRNA in a final volume of 20 μL of P3 electroporation buffer. The mixture was transferred to the corresponding wells of a 96-well Nucleofector™ plate (Lonza Catalog #V4SP-3960). Cells were electroporated in duplicate using Lonza shuttle 96w using manufacturer's pulse code. Immediately post electroporation, cells were recovered in 80 μL of TCGM without cytokines at 37° C. for 15 minutes. Electroporated T cells were subsequently cultured in TCGM further supplemented with 2× cytokines (200 U/mL recombinant human interleukin-2 (Peprotech, Cat. 200-02), 5 ng/ml recombinant human interleukin 7 (Peprotech, Cat. 200-07), and 5 ng/ml recombinant human interleukin 15 (Peprotech, Cat. 200-15) per well. The plates were incubated at 37° C. On day 4 post-electroporation, edited T cells were collected for NGS analysis. Fresh CTS Optimizer media (ThermoFisher Cat. A1048501) supplemented with 1× cytokines (200 U/mL recombinant human interleukin-2 (Peprotech, Cat. 200-02), 5 ng/ml recombinant human interleukin 7 (Peprotech, Cat. 200-07), and 5 ng/ml recombinant human interleukin 15 (Peprotech, Cat. 200-15) was added to the remaining cells and incubated at 37° C. degrees. On day 7 post-electroporation, edited T cells were collected for flow cytometry analysis.

1.5 RNA Electroporation of an sgRNA Dilution Series

Solutions containing sgRNAs targeting a gene of interest and solutions containing mRNA encoding Nme2 BC22n (SEQ ID No: 822) and mRNA encoding UGI (SEQ ID No. 821) were prepared in P3 electroporation buffer. Single guide RNAs were removed from their storage plates, denatured for 2 minutes at 95° C. and incubated at room temperature for 5 minutes. Forty-eight hours post-activation, T cells were harvested, centrifuged, and resuspended at a concentration of 12.5×10⁶ T cells/mL in P3 electroporation buffer (Lonza Catalog #V4SP-3960). Each sgRNA was serially diluted 1:2 (v/v) in P3 electroporation buffer starting from a final concentration of 5 μM. Subsequently, 1×10⁵ T cells, 20 ng/μL of BC22n mRNA and 20 ng/μL of UGI mRNA were added to serially diluted sgRNAs in a final volume of 20 μL of P3 electroporation buffer. The resulting mix was transferred in duplicate to 96-well Nucleofector™ plates. Cells were electroporated using the manufacturer's pulse code. Immediately post electroporation, cells were recovered in 80 μL of TCGM without cytokines at 37° C. for 15 minutes. After recovery, 80 μL of electroporated T cells were transferred to 96-well plates containing 80 μL of TCGM supplemented with 400 U/mL recombinant human interleukin-2 (Peprotech, Cat. 200-02), 10 ng/ml recombinant human interleukin 7 (Peprotech, Cat. 200-07), and 10 ng/ml recombinant human interleukin 15 (Peprotech, Cat. 200-15) per well. Plates were incubated at 37° C. for 4 days. On day 4 post-editing, T cells were sub-cultured 1:4 (v/v) and on day 7 post-editing, T cells were collected for flow cytometry analysis.

### 1.6 Lipid Nanoparticle Formulation

In general, the lipid nanoparticle (LNP) components were dissolved in 100% ethanol at various molar ratios. The RNA cargos (e.g., Cas9 or base editor mRNA and sgRNA) were dissolved in 25 mM citrate, 100 mM NaCl, pH 5.0, resulting in a concentration of RNA cargo of approximately 0.45 mg/mL. The LNPs used contained ionizable lipid ((9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl(9Z,12Z)-octadeca-9,12-dienoate), also called herein Lipid A, cholesterol, distearoylphosphatidylcholine (DSPC), and 1,2-dimyristoyl-rac-glycero-3-methoxypolyethylene glycol-2000 (PEG2K-DMG) (catalog #GM-020 from NOF, Tokyo, Japan) in a molar ratio of 35 Lipid A: 47.5 cholesterol: 15 DSPC: 2.5 PEG2k-DMG. The LNPs were formulated with a lipid amine to RNA phosphate (N:P) molar ratio of about 6. In some cases, LNPs were prepared with a single RNA species such as a mRNA or a gRNA. In some cases, LNPs were prepared with a mixture of mRNA and a guide RNA.

The LNPs were prepared using a cross-flow technique utilizing impinging jet mixing of the lipid in ethanol with two volumes of RNA solution and one volume of water. First, the lipid in ethanol was mixed through a mixing cross with the two volumes of RNA solution. Then, a fourth stream of water was mixed with the outlet stream of the cross through an inline tee (See WO 2016/010840, FIG. 2). The LNPs were held for at least 1 hour at room temperature, and further diluted with water (approximately 1:1 v/v). Diluted LNPs were buffer exchanged into 50 mM Tris, 45 mM NaCl, 5% (w/v) sucrose, pH 7.5 (TSS) and concentrated as needed by methods known in the art. The resulting mixture was then filtered using a 0.2 m sterile filter. The final LNPs were characterized to determine the encapsulation efficiency, polydispersity index, and average particle size. The final LNP was stored at 4° C. or −80° C. until further use.

### Example 2: Screening of HLA-A Guide RNAs with Nme2 BC22n

Guide RNAs designed for the disruption of the HLA-A gene were screened for editing efficacy in T cells by assessing loss of two allelic versions of the MHC I surface protein, HLA-A2 and HLA-A3. The donor had a heterozygous HLA-A phenotype of A*02:01:01G and 03:01:01G. The percentage of T cells double negative for HLA-A2 and A3 (“% A2-/A3-”) was determined by flow cytometry following editing at the HLA-A locus by electroporation with Nme2 base editor system and each test guide.

2.1 T Cell Preparation and Editing with RNA Electroporation

T cells from a single donor (no. 808) were prepared and activated as described in Example 1. T cells were electroporated with sgRNAs targeting HLA-A, mRNA encoding Nme2 BC22n SEQ ID NO: 822), and mRNA encoding UGI (SEQ ID NO: 821) as described in Example 1 and herein.

### 2.2 Flow Cytometry

On day 7 post-electroporation, edited T cells were phenotyped by flow cytometry to determine HLA-A2 and HLA-A3 protein expression. Briefly, T cells were incubated for 30 min at 4° C. with a mixture of antibodies against CD3 (BioLegend, Cat. No. 316314), CD4 (BioLegend, Cat. No. 317434), CD8 (BioLegend, Cat. No. 301046), Viakrome (Immunotech, Cat. No. C36628), HLA A2 (BioLegend, Cat. No. 343306), and HLA A3 (Fisher Scientific, Cat. No. 501122330) diluted at 1:100, and HLA B7 (Milteny Biotech, Cat. No. 130-120-234) diluted at 1:200 in cell staining buffer. Cells were subsequently washed and resuspended in 100 μL of cell staining buffer. Cells were then processed on a Cytoflex flow cytometer (Beckman Coulter) and analyzed using the FlowJo™ software package. T cells were gated based on size, shape, viability, and CD8 positivity.

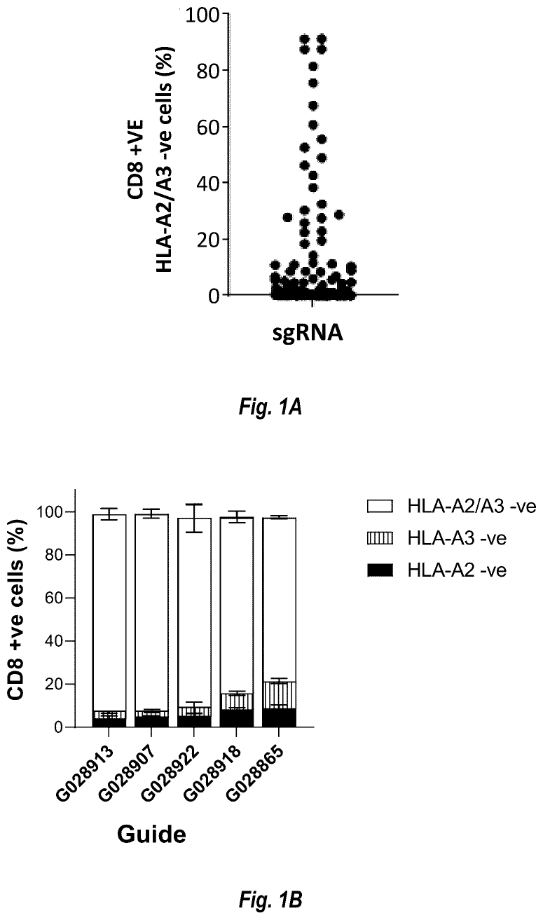

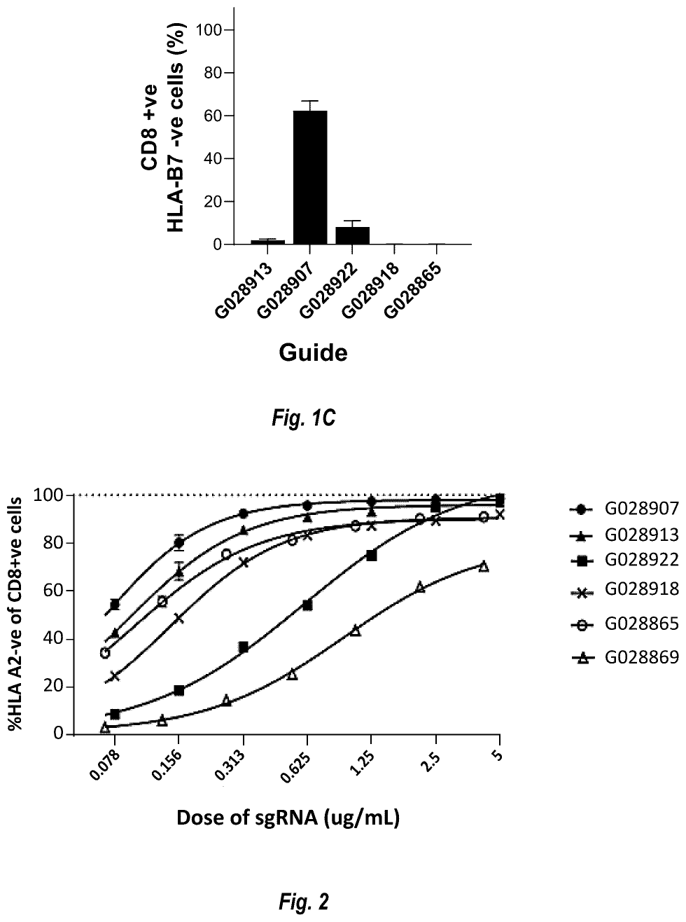

Table 8 and FIG. 1A show the mean percentage of cells double negative for HLA-A2 and HLA-A3 following editing at the HLA-A locus. The percentage of cells that are double or single negative for HLA-A2 and HLA-B3 for select guides, and guide specificity of the select guides to HLA-A over HLA-B are shown in FIGS. 1B and 1C.

TABLE 8
Mean percentage of T cells HLA-A negative (double negative
for HLA-A2 and HLA-A3) following editing at the HLA-A locus

| Guide | % HLA A2/A3 −ve | | |
|---|---|---|---|
| ID | Mean | SD | N |
| G028854 | 0.63 | 0.47 | 2 |
| G028855 | 14.15 | 0.64 | 2 |
| G028856 | 0.62 | 0.01 | 2 |
| G028857 | 1.32 | 0.35 | 2 |
| G028858 | 5.17 | 0.45 | 2 |
| G028859 | 4.51 | 0.59 | 2 |
| G028860 | 1.49 | 0.3 | 2 |
| G028861 | 4.26 | 0.18 | 2 |
| G028862 | 0.21 | 0.04 | 2 |
| G028863 | 0.08 | 0 | 2 |
| G028864 | 0.16 | 0.05 | 2 |
| G028865 | 75.45 | 0.78 | 2 |
| G028866 | 42.6 | 1.7 | 2 |
| G028867 | 11.6 | 1.41 | 2 |
| G028868 | 0.84 | 0.27 | 2 |
| G028869 | 38.3 | 2.4 | 2 |
| G028870 | 2.59 | 0.28 | 2 |
| G028871 | 0.19 | 0.03 | 2 |
| G028872 | 0.07 | 0.03 | 2 |
| G028873 | 0.03 | 0.01 | 2 |
| G028874 | 28.65 | 1.48 | 2 |
| G028875 | 0.37 | 0.14 | 2 |
| G028876 | 0.57 | 0.05 | 2 |
| G028877 | 10.9 | 0.57 | 2 |
| G028878 | 3.59 | 0.01 | 2 |
| G028879 | 1.73 | 0.23 | 2 |
| G028880 | 0.41 | 0.01 | 2 |
| G028881 | 0.06 | 0.03 | 2 |
| G028882 | 0.04 | 0.01 | 2 |
| G028883 | 0.27 | 0.01 | 2 |
| G028884 | 0.01 | 0.01 | 2 |
| G028885 | 0 | 0.01 | 2 |
| G028886 | 2.83 | 1.07 | 2 |
| G028887 | 0.84 | 0.62 | 2 |
| G028888 | 11.25 | 0.21 | 2 |
| G028889 | 0.91 | 0.07 | 2 |
| G028890 | 1.15 | 0.23 | 2 |
| G028891 | 25.7 | 1.84 | 2 |
| G028892 | 5.53 | 0.28 | 2 |
| G028893 | 1.57 | 0.18 | 2 |
| G028894 | 60.6 | 4.38 | 2 |
| G028895 | 1.02 | 0.1 | 2 |
| G028896 | 5.39 | 0.61 | 2 |
| G028897 | 0.37 | 0 | 2 |
| G028898 | 0.28 | 0.15 | 2 |
| G028899 | 52.5 | 5.09 | 2 |
| G028900 | 19.5 | 2.12 | 2 |
| G028901 | 27.45 | 0.49 | 2 |
| G028902 | 22.85 | 0.64 | 2 |
| G028903 | 55.5 | 3.54 | 2 |
| G028904 | 1.92 | 0.02 | 2 |
| G028905 | 6.86 | 0.88 | 2 |
| G028906 | 5.88 | 0.64 | 2 |
| G028907 | 91.1 | 2.26 | 2 |
| G028908 | 8.82 | 0.08 | 2 |
| G028909 | 1.05 | 0.23 | 2 |
| G028910 | 2.43 | 1.03 | 2 |
| G028911 | 0.02 | 0.03 | 2 |
| G028912 | 18.3 | 4.67 | 2 |
| G028913 | 91.15 | 2.76 | 2 |
| G028914 | 30.25 | 2.62 | 2 |
| G028915 | 8.47 | 1.39 | 2 |
| G028916 | 0.59 | 0.21 | 2 |
| G028917 | 0.01 | 0.01 | 2 |
| G028918 | 81.4 | 2.69 | 2 |
| G028919 | 0.49 | 0.44 | 2 |
| G028920 | 22.4 | 2.69 | 2 |
| G028921 | 2.12 | 0.31 | 2 |
| G028922 | 87.5 | 6.51 | 2 |
| G028923 | 67.45 | 13.51 | 2 |
| G028924 | 46.2 | 3.39 | 2 |
| G028925 | 0.07 | 0.06 | 2 |
| G028926 | 0.07 | 0.03 | 2 |
| G028927 | 0.86 | 0.06 | 2 |
| G028928 | 8.48 | 0.93 | 2 |
| G028929 | 2.9 | 0.32 | 2 |
| G028930 | 0.08 | 0.11 | 2 |
| G028933 | 8.31 | 1.34 | 2 |
| G028934 | 27.7 | 1.7 | 2 |

### Example 3: Dose Response Curves (DRC) for Select HLA-A Nme2 Guides

A titration experiment was conducted to evaluate the editing efficacy of sgRNAs designed for the disruption of the HLA-A locus by assessing loss of one allelic version of the MHC I surface protein, HLA-A2. An eight-point dose response curve was generated for each sgRNA by titrating each sgRNA with a fixed concentration of mRNA encoding Nme2 BC22n (SEQ ID NO:822) and mRNA encoding UGI (SEQ ID NO: 821) for electroporation in T cells. T cells were then expanded and phenotyped by flow cytometry to determine the editing efficiency of each sgRNA tested.

T cells were prepared as described in Example 1. T cells were edited with serially diluted sgRNAs by the mRNA electroporation method described in Example 1. On day 7 post-editing, edited T cells were phenotyped by flow cytometry to determine HLA-A2 protein expression. Briefly, T cells were transferred to U-bottom plates, spun at 500 g for 5 minutes and resuspended in 100 μL of FACs buffer (PBS with 2% FBS and 2 mM EDTA) containing the following staining reagents: PerCP/Cy5.5 anti-human CD3 (BioLegend, Cat. 317434) diluted 1:100 (v/v), BV421 anti-human CD4 (BioLegend, Cat. 317434) diluted 1:100 (v/v), BV785 anti-human CD8 (BioLegend, Cat. 301046) diluted 1:100 (v/v), PE anti-human HLA-A2 (BioLegend Inc., Cat. 343306) diluted 1:100 (v/v), FITC anti-human-HLA-B7 (Miltenyi Biotec Inc., Cat. 130-120-234) diluted 1:200 and Viakrome 808 (Beckman Coulter Cat. C36628) diluted 1:100. T cells were incubated in staining solution for 30 minutes at 4° C., washed and resuspended using 100 μL of FACS buffer. Then, T cells were processed in a Beckman Coulter Cytoflex LX flow cytometer, and analyzed using the FlowJo™ software package. T cells were gated based on size, singularity, viability and CD8 positivity. Table 9 and FIG. 2 show the percentage of CD8+ T cells that are negative for the expression of HLA-A2.

TABLE 9
Mean percentage of CD8⁺ HLA-A2⁻ T cells following base editing

| | G028907 | | | G028913 | | | G028869 | | |
|---|---|---|---|---|---|---|---|---|---|
| Guide | Mean | SD | | Mean | SD | | Mean | SD | |
| sgRNA | % CD8⁺ | % CD8⁺ | | % CD8⁺ | % CD8⁺ | | % CD8⁺ | % CD8⁺ | |
| (μg/mL) | Cells | Cells | N | Cells | Cells | N | Cells | Cells | N |
| 5 | 98.65 | 0.07 | 2 | 97.20 | 0.42 | 2 | 70.55 | 0.49 | 2 |
| 2.5 | 98.25 | 0.21 | 2 | 96.15 | 0.07 | 2 | 61.75 | 0.92 | 2 |
| 1.25 | 97.40 | 0.14 | 2 | 93.30 | 0.00 | 2 | 43.75 | 1.91 | 2 |
| 0.625 | 95.70 | 0.42 | 2 | 90.85 | 0.92 | 2 | 25.45 | 0.92 | 2 |
| 0.313 | 92.40 | 0.00 | 2 | 85.55 | 1.77 | 2 | 14.55 | 0.92 | 2 |
| 0.078 | 80.20 | 3.25 | 2 | 68.25 | 3.75 | 2 | 6.20 | 0.33 | 2 |
| 0 | 54.45 | 2.05 | 2 | 42.70 | 1.13 | 2 | 3.19 | 0.40 | 2 |
| EC50 | 0.07038 | | | 0.08998 | | | 0.9213 | | |
| | G028922 | | | G028918 | | | G028865 | | |
| Guide | Mean | SD | | Mean | SD | | Mean | SD | |
| sgRNA | % CD8⁺ | % CD8⁺ | | % CD8⁺ | % CD8⁺ | | % CD8⁺ | % CD8⁺ | |
| (μg/mL) | Cells | Cells | N | Cells | Cells | N | Cells | Cells | N |
| 5 | 98.65 | 0.21 | 2 | 92.15 | 0.07 | 2 | 90.95 | 1.06 | 2 |
| 2.5 | 95.00 | 0.99 | 2 | 89.45 | 1.20 | 2 | 90.20 | 1.27 | 2 |
| 1.25 | 74.90 | 2.12 | 2 | 87.25 | 1.06 | 2 | 87.20 | 0.42 | 2 |
| 0.625 | 54.20 | 0.42 | 2 | 83.40 | 0.71 | 2 | 81.35 | 1.34 | 2 |
| 0.313 | 36.80 | 0.85 | 2 | 72.05 | 0.64 | 2 | 75.45 | 1.06 | 2 |
| 0.078 | 18.60 | 0.42 | 2 | 48.80 | 0.85 | 2 | 55.60 | 2.26 | 2 |
| 0 | 8.73 | 0.02 | 2 | 24.65 | 1.20 | 2 | 34.30 | 1.56 | 2 |

| | | | |
|---|---|---|---|
| EC50 | 0.6028 | 0.1429 | 0.09631 |

### Example 4: Screening of TRAC Guide RNAs with Nme2 Base Editing System

TRAC guide RNAs were screened for Nme2 base editing efficacy in T cells by assessing loss of CD3 cell surface expression by flow cytometry and editing frequency by NGS. CD3 is a cell-surface component of the T cell receptor complex and its presence at the cell surface is used as a surrogate marker for TRAC protein expression.

4.1 T Cell Preparation and Editing with RNA Electroporation

T cells were prepared and activated as described in Example 1. T cells were electroporated with sgRNAs targeting TRAC, mRNA encoding Nme2 BC22n (SEQ ID NO: 822), and mRNA encoding UGI (SEQ ID NO: 821) as described in Example 1 and herein.

### 4.2 Flow Cytometry and NGS Sequencing

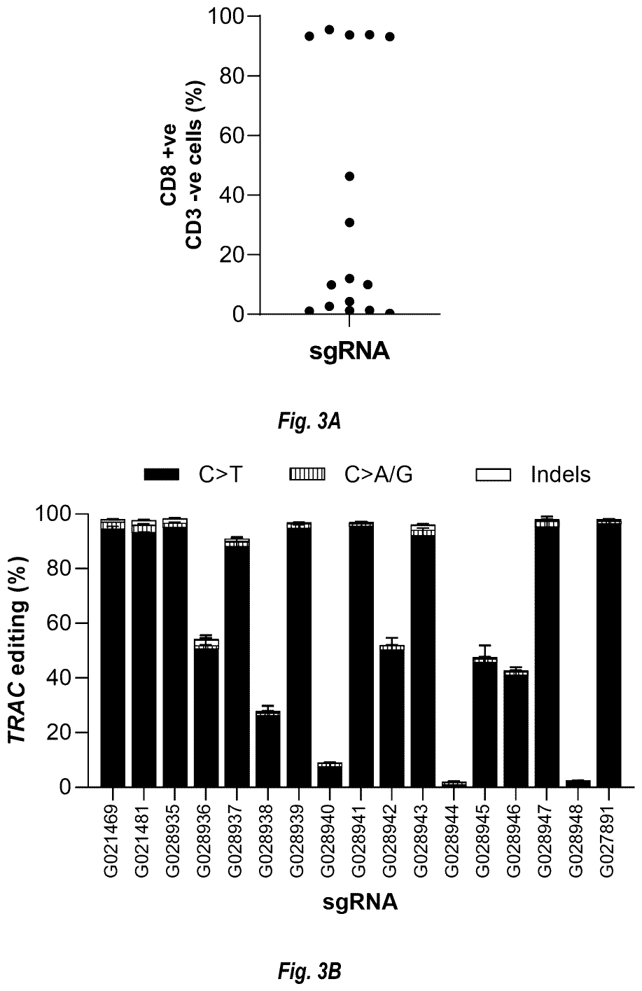

T cells from a single donor (no. 3786) were prepared as described in Example 1. T cells were edited with sgRNA targeting the TRAC locus using mRNA electroporation as described in Example 1. On day 4 post-electroporation, edited T cell samples were subjected to PCR and NGS analysis as described in Example 1. On day 7 post-electroporation, T cells were phenotyped by flow cytometry. Briefly, T cells were incubated for 30 min at 4° C. with a mixture of antibodies against CD3 (BioLegend, Cat. No. 316314), CD4 (BioLegend, Cat. No. 317434), CD8 (BioLegend, Cat. No. 301046), Viakrome (Immunotech, Cat. No. C36628) diluted at 1:100 in cell staining buffer (BioLegend, Cat. No. 420201). Cells were subsequently washed and resuspended in 100 μL of cell staining buffer. Cells were then processed on a Cytoflex flow cytometer (Beckman Coulter) and analyzed using the FlowJo™ software package. T cells were gated based on size, shape, viability, CD8 and CD3 expression. Table 10 and FIG. 3A show the mean percentage of CD3 negative T cells. Mean percent editing in is shown in Table 10 and FIG. 3B.

TABLE 10
Mean percentage of CD3 negative T cells; Mean percent editing
of the TRAC loci as a percentage of total NGS reads. N =
4 represents two technical replicates each with two primer sets.

| | % CD3 −ve | | | % C to T | | | % C to A/G | | | % Indels | | |
|---|---|---|---|---|---|---|---|---|---|---|---|---|
| Guide ID | Mean | SD | N | Mean | SD | N | Mean | SD | N | Mean | SD | N |
| G021469 | 93.25 | 1.20 | 2 | 94.59 | 0.81 | 4 | 2.83 | 0.64 | 4 | 0.72 | 0.09 | 4 |
| G021481 | 95.50 | 1.56 | 2 | 93.52 | 0.31 | 4 | 2.59 | 0.14 | 4 | 1.75 | 0.22 | 4 |
| G028935 | 93.75 | 1.20 | 2 | 95.22 | 0.34 | 4 | 1.67 | 0.35 | 4 | 1.65 | 0.14 | 4 |
| G028936 | 2.66 | 0.73 | 2 | 50.63 | 5.08 | 4 | 1.26 | 0.37 | 4 | 2.48 | 0.35 | 4 |
| G028937 | 46.25 | 8.13 | 2 | 88.13 | 2.82 | 4 | 2.04 | 1.03 | 4 | 0.87 | 0.57 | 4 |
| G028938 | 9.93 | 0.24 | 2 | 26.77 | 3.16 | 4 | 0.91 | 0.06 | 4 | 0.35 | 0.05 | 4 |
| G028939 | 93.10 | 0.99 | 2 | 95.12 | 0.97 | 4 | 1.28 | 0.18 | 4 | 0.55 | 0.08 | 4 |
| G028940 | 1.28 | 0.01 | 2 | 7.76 | 0.85 | 4 | 1.22 | 0.10 | 4 | 0.21 | 0.06 | 4 |
| G028941 | 11.95 | 0.21 | 2 | 95.88 | 1.30 | 4 | 0.92 | 0.19 | 4 | 0.39 | 0.07 | 4 |
| G028942 | 30.75 | 5.73 | 2 | 50.37 | 4.35 | 4 | 1.47 | 0.24 | 4 | 0.21 | 0.08 | 4 |
| G028943 | 93.65 | 5.44 | 2 | 92.43 | 3.16 | 4 | 1.94 | 0.62 | 4 | 1.80 | 0.33 | 4 |
| G028944 | 1.25 | 0.19 | 2 | 1.42 | 0.10 | 4 | 0.73 | 0.11 | 4 | 0.12 | 0.04 | 4 |
| G028945 | 9.89 | 1.86 | 2 | 46.15 | 5.94 | 4 | 1.01 | 0.07 | 4 | 0.58 | 0.11 | 4 |
| G028946 | 4.29 | 0.11 | 2 | 41.35 | 2.67 | 4 | 0.88 | 0.45 | 4 | 0.66 | 0.10 | 4 |
| G028947 | 1.05 | 0.08 | 2 | 95.68 | 1.72 | 4 | 1.83 | 1.65 | 4 | 0.63 | 0.05 | 4 |
| G028948 | 0.27 | 0.19 | 2 | 1.83 | 0.14 | 4 | 0.56 | 0.07 | 4 | 0.20 | 0.09 | 4 |

### Example 5: Dose Response Curves (DRC) for Select TRAC Nme2 Guides

A titration experiment was conducted to evaluate the editing efficacy of select sgRNAs that target the TRAC locus by assessing loss of CD3 cell surface expression. CD3 is a cell-surface component of the T cell receptor complex and its presence at the cell surface is used as a surrogate marker for TRAC protein expression. An eight-point dose response curve was generated for each sgRNA by titrating each sgRNA with a fixed concentration of Nme2 BC22n (SEQ ID NO:822) and UGI mRNA132 (SEQ ID NO: 821) in T cells using electroporation. T cells were then expanded and phenotyped by flow cytometry to determine the editing efficiency of each sgRNA tested.

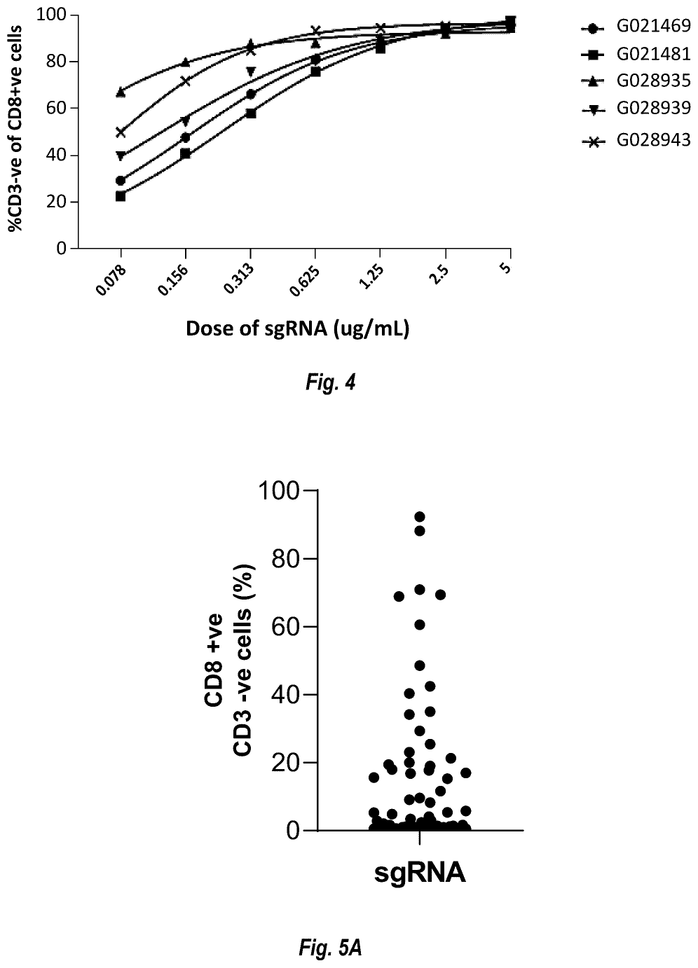

T cells were prepared as described in Example 1. T cells were edited with serially diluted sgRNAs that target TRAC by mRNA electroporation as described in Example 1. On day 7 post-editing, edited T cells were phenotyped by flow cytometry to determine CD3 expression as described in Example 4. T cells were gated based on size, singularity, viability and CD8 positivity. Tables 11 and FIG. 4 show the percentage of CD8+ T cells that are negative for the expression of CD3.

TABLE 11
Mean percent CD3 negative T cells following
base editing at the TRAC locus

| G021469 | | | | G021481 | | | |
|---|---|---|---|---|---|---|---|
| sgRNA | % CD3− of CD8+ | | | sgRNA | % CD3− of CD8+ | | |
| (uM) | Ave | SD | N | (uM) | Ave | SD | N |
| 5.00 | 95.40 | 0.00 | 2.00 | 5.00 | 97.35 | 0.21 | 2 |
| 2.50 | 92.20 | 0.85 | 2.00 | 2.50 | 94.15 | 0.21 | 2 |
| 1.25 | 88.45 | 2.90 | 2.00 | 1.25 | 85.6 | 0.42 | 2 |
| 0.63 | 78.55 | 3.46 | 2.00 | 0.63 | 74.75 | 1.48 | 2 |
| 0.31 | 64.80 | 1.84 | 2.00 | 0.31 | 54.75 | 4.45 | 2 |
| 0.16 | 47.90 | 0.42 | 2.00 | 0.16 | 39.9 | 1.27 | 2 |
| 0.08 | 25.40 | 5.37 | 2.00 | 0.08 | 22.05 | 0.64 | 2 |
| 0.00 | 0.47 | 0.34 | 2.00 | 0.00 | 0.375 | 0.06 | 2 |
| G028935 | | | | G028939 | | | |
| sgRNA | % CD3− of CD8+ | | | sgRNA | % CD3− of CD8+ | | |
| (uM) | Ave | SD | N | (uM) | Ave | SD | N |
| 5.00 | 94.60 | 0.14 | 2.00 | 5.00 | 97.7 | 0.42 | 2 |
| 2.50 | 92.10 | 0.14 | 2.00 | 2.50 | 92.65 | 1.20 | 2 |
| 1.25 | 90.05 | 0.07 | 2.00 | 1.25 | 88.35 | 0.49 | 2 |
| 0.63 | 88.15 | 1.20 | 2.00 | 0.63 | 79.55 | 1.06 | 2 |
| 0.31 | 87.75 | 0.78 | 2.00 | 0.31 | 73.35 | 3.04 | 2 |
| 0.16 | 79.90 | 1.27 | 2.00 | 0.16 | 51.8 | 3.39 | 2 |
| 0.08 | 67.20 | 1.13 | 2.00 | 0.08 | 36.05 | 5.02 | 2 |
| 0.00 | 0.46 | 0.23 | 2.00 | 0.00 | 0.22 | 0.03 | 2 |

G028943

| sgRNA | % CD3− of CD8+ | | |
|---|---|---|---|
| (uM) | Ave | SD | N |
| 5.00 | 97.50 | 0.71 | 2 |
| 2.50 | 95.55 | 0.64 | 2 |
| 1.25 | 91.95 | 3.46 | 2 |
| 0.63 | 91.10 | 2.97 | 2 |
| 0.31 | 85.40 | 0.71 | 2 |
| 0.16 | 70.05 | 2.33 | 2 |
| 0.08 | 50.10 | 0.28 | 2 |
| 0.00 | 0.40 | 0.04 | 2 |

### Example 6: Screening of TRBC 1 and 2 Guide RNAs with Nme2 BC22n

Guide RNAs targeting TRBC 1 and/or TRBC 2 were screened for editing efficacy in T cells by assessing loss of CD3 cell surface expression by flow cytometry and for editing efficacy by NGS, following TRBC editing by mRNA delivery. CD3 is a cell-surface component of the T cell receptor complex and its presence at the cell surface is used as a surrogate marker for TRBC protein expression.

6.1 T Cell Preparation and Editing with RNA Electroporation

T cells were prepared and activated as described in Example 1. T cells were electroporated with sgRNAs targeting TRBC, mRNA encoding Nme2 BC22n (SEQ ID NO:822), and mRNA encoding UGI (SEQ ID NO: 821) as described in Example 1 and herein.

### 6.2 Flow Cytometry and NGS Sequencing

T cells from a single donor (no. 3786) were prepared as described in Example 1. T cells were edited with sgRNA targeting the TRBC1 and/or TRBC 2 locus using mRNA electroporation as described in Example 1. On day 4 post-electroporation, edited T cell samples were subjected PCR and NGS analysis as described in Example 1. On day 7 post-electroporation, T cells were phenotyped by flow cytometry. Briefly, T cells were incubated for 30 min at 4° C. with a mixture of antibodies against CD3 (BioLegend, Cat. No. 316314), CD4 (BioLegend, Cat. No. 317434), CD8 (BioLegend, Cat. No. 301046), and Viakrome (Immunotech, Cat. No. C36628) diluted at 1:100 in cell staining buffer (BioLegend, Cat. No. 420201). Cells were subsequently washed and resuspended in 100 μL of cell staining buffer. Cells were then processed on a Cytoflex flow cytometer (Beckman Coulter) and analyzed using the FlowJo™ software package. T cells were gated based on size, shape, viability, CD8 and CD3 expression.

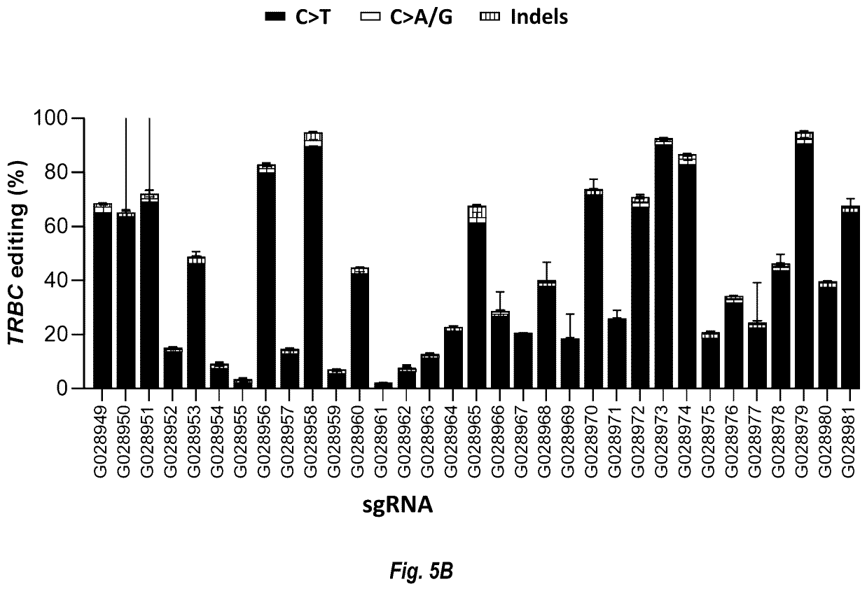

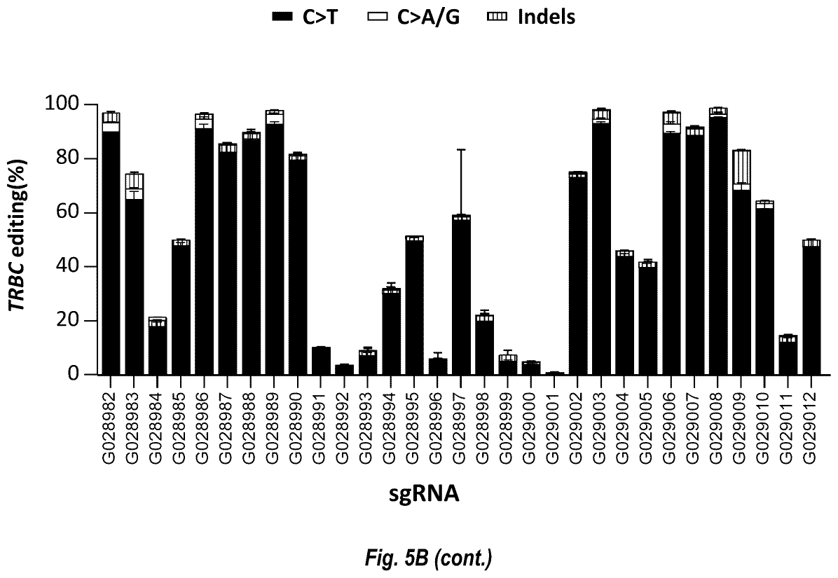

Table 12 and FIG. 5A show the mean percentage of CD3− T cells. Percent editing at the TRBC 1 and/or 2 locus is shown in Table 12 and in FIG. 5B.

TABLE 12
Mean percentage of CD3 negative T cells; Mean percent editing
of the TRBC loci as a percentage of total NGS reads.

| | % CD3 -ve | | | % C to T | | | % C to A/G | | | % Indels | | | Gene |
|---|---|---|---|---|---|---|---|---|---|---|---|---|---|
| Guide ID | Mean | SD | N | Mean | SD | N | Mean | SD | N | Mean | SD | N | target |
| G028952 | 9.65 | 1.06 | 2 | 14.08 | 1.30 | 4 | 0.84 | 0.50 | 4 | 0.36 | 0.06 | 4 | TRBC1 |
| G028953 | 15.30 | 0.99 | 2 | 46.47 | 4.15 | 4 | 1.82 | 0.48 | 4 | 0.61 | 0.12 | 4 | TRBC1 |
| G028968 | 19.45 | 2.05 | 2 | 38.11 | 8.64 | 4 | 1.29 | 0.21 | 4 | 0.74 | 0.04 | 4 | TRBC1 |
| G028969 | 3.41 | 0.49 | 2 | 17.85 | 9.69 | 4 | 0.63 | 0.39 | 4 | 0.15 | 0.05 | 4 | TRBC1 |
| G028970 | 29.30 | 3.54 | 2 | 71.73 | 5.73 | 4 | 1.59 | 0.33 | 4 | 0.54 | 0.08 | 4 | TRBC1 |
| G028977 | 0.99 | 0.07 | 2 | 23.09 | 16.10 | 4 | 0.90 | 1.04 | 4 | 0.49 | 0.59 | 4 | TRBC1 |
| G028978 | 1.67 | 0.25 | 2 | 43.93 | 5.74 | 4 | 1.46 | 0.09 | 4 | 0.82 | 0.24 | 4 | TRBC1 |
| G028979 | 5.32 | 0.29 | 2 | 90.62 | 2.52 | 4 | 2.11 | 0.18 | 4 | 2.32 | 0.28 | 4 | TRBC1 |
| G028980 | 2.41 | 0.00 | 2 | 37.84 | 1.93 | 4 | 1.57 | 0.28 | 4 | 0.36 | 0.10 | 4 | TRBC1 |
| G028981 | 1.08 | ND | 1 | 65.21 | 5.04 | 4 | 1.99 | 0.24 | 4 | 0.55 | 0.03 | 4 | TRBC1 |
| G028982 | 15.60 | 0.14 | 2 | 89.82 | 1.24 | 4 | 3.90 | 0.33 | 4 | 3.30 | 0.57 | 4 | TRBC1 |
| G028983 | 1.42 | 0.35 | 2 | 65.07 | 2.82 | 4 | 4.05 | 0.28 | 4 | 5.47 | 0.59 | 4 | TRBC1 |
| G028984 | 0.22 | 0.08 | 2 | 18.19 | 1.46 | 4 | 1.92 | 0.32 | 4 | 1.14 | 0.17 | 4 | TRBC1 |
| G028985 | 5.78 | 0.12 | 2 | 47.94 | 1.02 | 4 | 1.77 | 0.22 | 4 | 0.45 | 0.15 | 4 | TRBC1 |
| G028986 | 92.30 | 4.53 | 2 | 91.48 | 1.28 | 4 | 3.58 | 0.49 | 4 | 1.83 | 0.17 | 4 | TRBC1 |
| G028987 | 68.85 | 4.45 | 2 | 82.47 | 2.97 | 4 | 2.65 | 0.14 | 4 | 0.59 | 0.16 | 4 | TRBC1 |
| G028989 | 19.00 | 0.57 | 2 | 92.81 | 0.70 | 4 | 3.76 | 0.30 | 4 | 1.43 | 0.16 | 4 | TRBC1 |
| G028990 | 0.73 | 0.25 | 2 | 79.63 | 2.77 | 4 | 1.72 | 0.21 | 4 | 0.75 | 0.19 | 4 | TRBC1 |
| G028991 | 0.31 | 0.26 | 2 | 9.78 | 0.55 | 4 | 0.58 | 0.21 | 4 | 0.10 | 0.03 | 4 | TRBC1 |
| G028992 | 0.19 | 0.08 | 2 | 3.26 | 0.29 | 4 | 0.45 | 0.30 | 4 | 0.10 | 0.04 | 4 | TRBC1 |
| G028993 | 0.16 | 0.16 | 2 | 7.36 | 0.89 | 4 | 1.57 | 1.38 | 4 | 0.56 | 0.42 | 4 | TRBC1 |
| G028949 | 62.95 | 6.43 | 2 | 65.32 | 3.28 | 4 | 2.23 | 0.26 | 4 | 0.98 | 0.14 | 4 | TRBC1/2 |
| G028950 | 65.60 | 4.38 | 2 | 63.75 | 44.44 | 4 | 1.13 | 1.30 | 4 | 0.40 | 0.47 | 4 | TRBC1/2 |
| G028951 | 70.90 | 1.27 | 2 | 69.08 | 46.09 | 4 | 1.25 | 0.83 | 4 | 1.87 | 1.25 | 4 | TRBC1/2 |
| G028954 | 1.48 | ND | 1 | 7.84 | 0.83 | 4 | 1.25 | 0.75 | 4 | 0.16 | 0.06 | 4 | TRBC1/2 |
| G028955 | 0.50 | 0.13 | 2 | 2.35 | 0.43 | 4 | 1.05 | 0.57 | 4 | 0.11 | 0.05 | 4 | TRBC1/2 |
| G028956 | 20.00 | 1.98 | 2 | 80.04 | 3.44 | 4 | 1.82 | 0.32 | 4 | 1.04 | 0.19 | 4 | TRBC1/2 |
| G028957 | 2.09 | 0.39 | 2 | 13.49 | 0.63 | 4 | 1.17 | 0.31 | 4 | 0.23 | 0.06 | 4 | TRBC1/2 |
| G028958 | 69.40 | 0.14 | 2 | 89.79 | 0.26 | 4 | 2.25 | 0.16 | 4 | 2.67 | 0.31 | 4 | TRBC1/2 |
| G028959 | 2.86 | 0.49 | 2 | 5.62 | 0.62 | 4 | 1.32 | 0.30 | 4 | 0.12 | 0.05 | 4 | TRBC1/2 |
| G028960 | 42.50 | 4.24 | 2 | 42.76 | 0.36 | 4 | 1.74 | 0.44 | 4 | 0.25 | 0.08 | 4 | TRBC1/2 |
| G028961 | 1.27 | 0.04 | 2 | 1.64 | 0.43 | 4 | 0.46 | 0.08 | 4 | 0.12 | 0.04 | 4 | TRBC1/2 |
| G028962 | 0.65 | 0.20 | 2 | 6.58 | 2.20 | 4 | 1.12 | 0.57 | 4 | 0.14 | 0.06 | 4 | TRBC1/2 |
| G028963 | 11.65 | 2.19 | 2 | 11.55 | 1.59 | 4 | 0.97 | 0.49 | 4 | 0.25 | 0.05 | 4 | TRBC1/2 |
| G028964 | 23.10 | 1.84 | 2 | 21.45 | 0.91 | 4 | 1.09 | 0.59 | 4 | 0.32 | 0.05 | 4 | TRBC1/2 |
| G028965 | 40.35 | 5.59 | 2 | 61.42 | 3.81 | 4 | 2.10 | 0.15 | 4 | 4.12 | 0.40 | 4 | TRBC1/2 |
| G028966 | 18.00 | 4.67 | 2 | 26.83 | 8.93 | 4 | 1.00 | 0.11 | 4 | 0.78 | 0.46 | 4 | TRBC1/2 |
| G028967 | 21.30 | 2.55 | 2 | 19.82 | 0.85 | 4 | 0.63 | 0.17 | 4 | 0.17 | 0.09 | 4 | TRBC1/2 |
| G028971 | 1.14 | 0.01 | 2 | 25.06 | 3.88 | 4 | 0.49 | 0.06 | 4 | 0.42 | 0.14 | 4 | TRBC1/2 |
| G028972 | 16.85 | 2.05 | 2 | 67.14 | 4.73 | 4 | 2.15 | 1.09 | 4 | 1.63 | 0.27 | 4 | TRBC1/2 |
| G028973 | 4.88 | 0.30 | 2 | 90.36 | 2.03 | 4 | 1.35 | 0.19 | 4 | 0.98 | 0.14 | 4 | TRBC1/2 |
| G028974 | 3.05 | 0.15 | 2 | 83.09 | 1.63 | 4 | 2.11 | 0.75 | 4 | 1.58 | 0.12 | 4 | TRBC1/2 |
| G028975 | 1.01 | 0.04 | 2 | 18.72 | 2.39 | 4 | 1.49 | 0.34 | 4 | 0.60 | 0.12 | 4 | TRBC1/2 |
| G028976 | 0.97 | 0.18 | 2 | 31.70 | 1.75 | 4 | 1.66 | 0.39 | 4 | 0.86 | 0.20 | 4 | TRBC1/2 |
| G028988 | 9.14 | 0.85 | 2 | 87.16 | 3.83 | 4 | 2.33 | 0.27 | 4 | 0.51 | 0.08 | 4 | TRBC1/2 |
| G028994 | 17.70 | 3.82 | 2 | 30.45 | 3.67 | 4 | 1.30 | 0.53 | 4 | 0.54 | 0.06 | 4 | TRBC2 |
| G028995 | 25.45 | 2.47 | 2 | 49.38 | 1.71 | 4 | 1.61 | 0.50 | 4 | 0.64 | 0.03 | 4 | TRBC2 |
| G028996 | 1.64 | 0.28 | 2 | 5.61 | 2.70 | 4 | 0.41 | 0.14 | 4 | 0.19 | 0.06 | 4 | TRBC2 |
| G028997 | 17.00 | 0.85 | 2 | 57.64 | 25.79 | 4 | 1.16 | 0.35 | 4 | 0.54 | 0.17 | 4 | TRBC2 |
| G028998 | 4.13 | 1.70 | 2 | 20.02 | 4.05 | 4 | 2.11 | 0.71 | 4 | 0.24 | 0.08 | 4 | TRBC2 |
| G028999 | 1.51 | 0.12 | 2 | 5.33 | 0.63 | 4 | 1.88 | 2.00 | 4 | 0.16 | 0.03 | 4 | TRBC2 |
| G029000 | 0.58 | 0.06 | 2 | 4.08 | 0.41 | 4 | 0.79 | 0.25 | 4 | 0.32 | 0.08 | 4 | TRBC2 |
| G029001 | 0.25 | 0.01 | 2 | 0.60 | 0.05 | 4 | 0.48 | 0.04 | 4 | 0.11 | 0.03 | 4 | TRBC2 |
| G029002 | 8.26 | 0.19 | 2 | 73.39 | 0.87 | 4 | 1.13 | 0.26 | 4 | 0.73 | 0.05 | 4 | TRBC2 |
| G029003 | 35.00 | 0.28 | 2 | 93.20 | 0.57 | 4 | 1.43 | 0.17 | 4 | 3.78 | 0.38 | 4 | TRBC2 |
| G029004 | 34.20 | 2.12 | 2 | 44.18 | 0.76 | 4 | 1.66 | 0.18 | 4 | 0.35 | 0.07 | 4 | TRBC2 |
| G029005 | 5.40 | 0.40 | 2 | 40.16 | 2.70 | 4 | 1.34 | 0.18 | 4 | 0.48 | 0.06 | 4 | TRBC2 |
| G029006 | 88.15 | 2.76 | 2 | 89.41 | 0.89 | 4 | 3.49 | 0.58 | 4 | 4.54 | 0.26 | 4 | TRBC2 |
| G029007 | 60.55 | 0.64 | 2 | 88.74 | 2.32 | 4 | 2.40 | 0.77 | 4 | 0.84 | 0.23 | 4 | TRBC2 |
| G029008 | 48.55 | 1.48 | 2 | 95.20 | 0.44 | 4 | 1.56 | 0.26 | 4 | 2.05 | 0.23 | 4 | TRBC2 |
| G029009 | 0.95 | 0.22 | 2 | 68.27 | 2.76 | 4 | 2.26 | 0.91 | 4 | 12.78 | 0.16 | 4 | TRBC2 |
| G029010 | 1.42 | 0.25 | 2 | 61.68 | 1.86 | 4 | 1.78 | 0.12 | 4 | 1.13 | 0.10 | 4 | TRBC2 |
| G029011 | 0.25 | 0.01 | 2 | 12.47 | 1.05 | 4 | 2.16 | 0.42 | 4 | 0.18 | 0.02 | 4 | TRBC2 |
| G029012 | 0.17 | 0.01 | 2 | 47.87 | 2.46 | 4 | 2.07 | 0.15 | 4 | 0.22 | 0.05 | 4 | TRBC2 |

“ND” indicates no data.
N = 4 represents two technical replicates each with two primer sets.

### Example 7: Dose Response Curves (DRC) for Select TRBC Nme2 Guides

A titration experiment was conducted to evaluate the editing efficacy of select sgRNAs designed for the disruption of one or both TRBC genes by assessing loss of CD3 cell surface expression. CD3 is a cell-surface component of the T cell receptor complex and its presence at the cell surface is used as a surrogate marker for TRBC protein expression. An eight-point dose response curve was generated for each sgRNA by titrating each sgRNA with a fixed concentration of mRNA encoding Nme2 BC22n (SEQ ID NO:822) and mRNA encoding UGI (SEQ ID NO: 1821 in T cells using electroporation. T cells were then expanded and phenotyped by flow cytometry to determine the editing efficiency of each sgRNA tested.

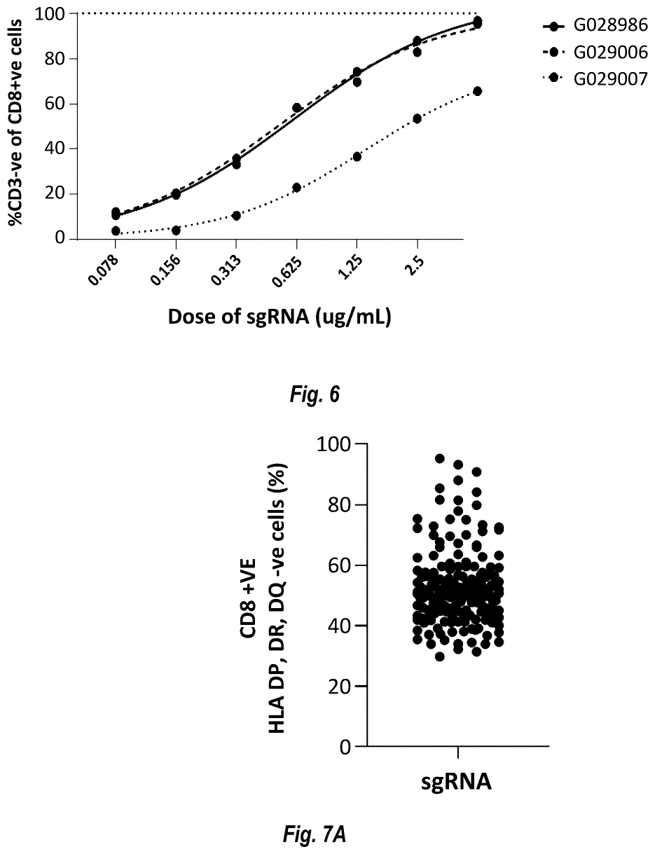

T cells were prepared as described in Example 1. T cells were edited with serially diluted TRBC targeting sgRNAs by mRNA electroporation as described in Example 1 and Table 13. On day 7 post-editing, edited T cells were phenotyped by flow cytometry to determine loss of CD3 expression as described in Example 4. T cells were gated based on size, singularity, viability and CD8 positivity. Table 13 and FIG. 6 show the percentage of CD8+ T cells that are negative for the expression of CD3.

T cells were prepared as described in Example 1. T cells were edited with serially diluted TRBC targeting sgRNAs by mRNA electroporation as described in Example 1. On day 7 post-editing, edited T cells were phenotyped by flow cytometry to determine loss of CD3 expression as described in Example 3. T cells were gated based on size, singularity, viability and CD8 positivity. Tables 13 and FIG. 6 show the percentage of CD8+ T cells that are negative for the expression of CD3.

TABLE 13
Percentage of CD8 positive cells that are
CD3 negative at various doses of TRBC sgRNA

| | % CD3− of | | |
|---|---|---|---|
| | CD8+ | | |
| sgRNA (uM) | Ave | SD | N |

G028986

| | | | |
|---|---|---|---|
| 5.00 | 96.75 | 0.07 | 2 |
| 2.50 | 88.15 | 0.35 | 2 |
| 1.25 | 70.85 | 1.63 | 2 |
| 0.63 | 56 | 3.25 | 2 |
| 0.31 | 34.35 | 1.48 | 2 |
| 0.16 | 20 | 0.28 | 2 |
| 0.08 | 10.16 | 1.05 | 2 |
| 0.00 | 0.36 | 0.06 | 2 |

G029006

| | | | |
|---|---|---|---|
| 5.00 | 95.05 | 0.35 | 2 |
| 2.50 | 82.75 | 0.21 | 2 |
| 1.25 | 68.35 | 8.27 | 2 |
| 0.63 | 55.05 | 4.60 | 2 |
| 0.31 | 37.55 | 2.33 | 2 |
| 0.16 | 19.7 | 1.13 | 2 |
| 0.08 | 13.1 | 1.13 | 2 |
| 0.00 | 0.43 | 0.00 | 2 |

G029007

| | | | |
|---|---|---|---|
| 5.00 | 65.65 | 0.07 | 2 |
| 2.50 | 52.55 | 1.34 | 2 |
| 1.25 | 36.3 | 0.57 | 2 |
| 0.63 | 22.6 | 0.71 | 2 |
| 0.31 | 10.4 | 0.42 | 2 |
| 0.16 | 4.565 | 0.49 | 2 |
| 0.08 | 3.21 | 1.09 | 2 |
| 0.00 | 0.335 | 0.05 | 2 |

### Example 8: Screening of CIITA Guide RNAs with Nme2 BC22n and Nme2 Cas9

8.1 Screen of CIITA sgRNA with Nme2 BC22n

CIITA guide RNAs were screened for editing efficacy in T cells by assessing loss of HLA DP, DQ, DR cell surface expression and editing by NGS, following CIITA editing by mRNA delivery. HLA-DP, DR and DQ are cell surface proteins whose expression requires the transcription factor CIITA and thus their presence at the cell surface is a surrogate marker for CIITA protein expression.

8.1.1 T Cell Preparation and Editing with RNA Electroporation

T cells from a single donor (no. 3786) were prepared and activated as described in Example 1. T cells were electroporated with sgRNAs targeting CIITA, mRNA encoding Nme2 BC22n (SEQ ID NO: 822), and mRNA encoding UGI (SEQ ID NO: 821) as described in Example 1 and herein.

### 8.1.2 Flow Cytometry and NGS Sequencing

T cells were prepared as described in Example 1. T cells were edited with sgRNA targeting the CIITA locus using mRNA electroporation as described in Example 1. On day 4 post-electroporation, edited T cell samples were subjected PCR and NGS analysis as described in Example 1. On day 7 post-electroporation, T cells were phenotyped by flow cytometry as described in Example 1 except the mixture of antibodies used were against CD3 (BioLegend, Cat. No. 316314), CD4 (BioLegend, Cat. No. 317434), CD8 (BioLegend, Cat. No. 301046), Viakrome (Immunotech, Cat. No. C36628) diluted at 1:100, and HLA II-DR, DP, DQ (BioLegend, Cat. No. 361706) diluted at 1:50 in cell staining buffer (BioLegend, Cat. No. 420201). T cells were gated based on size, shape, viability, CD8 and HLA II-DR, DP, DQ, expression.

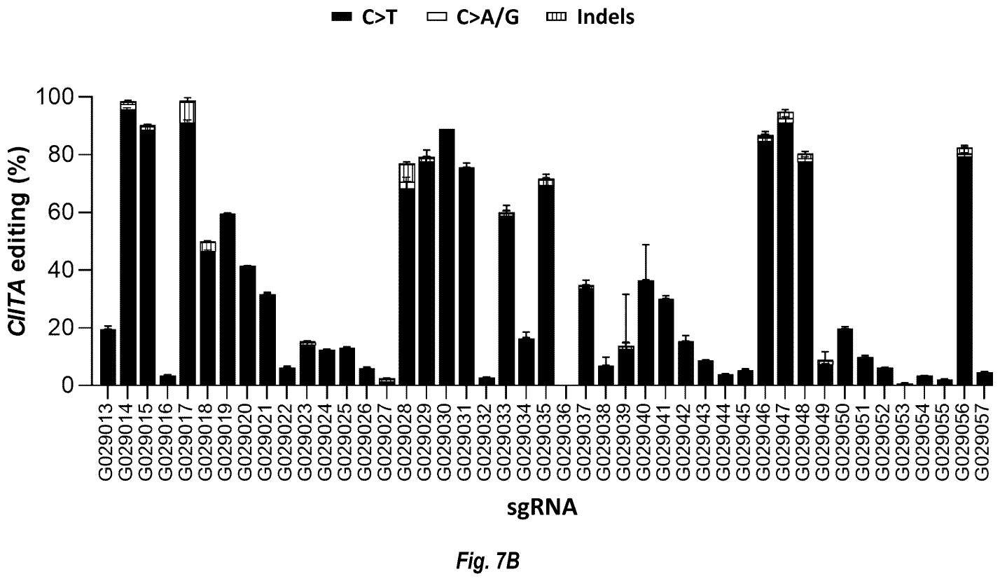

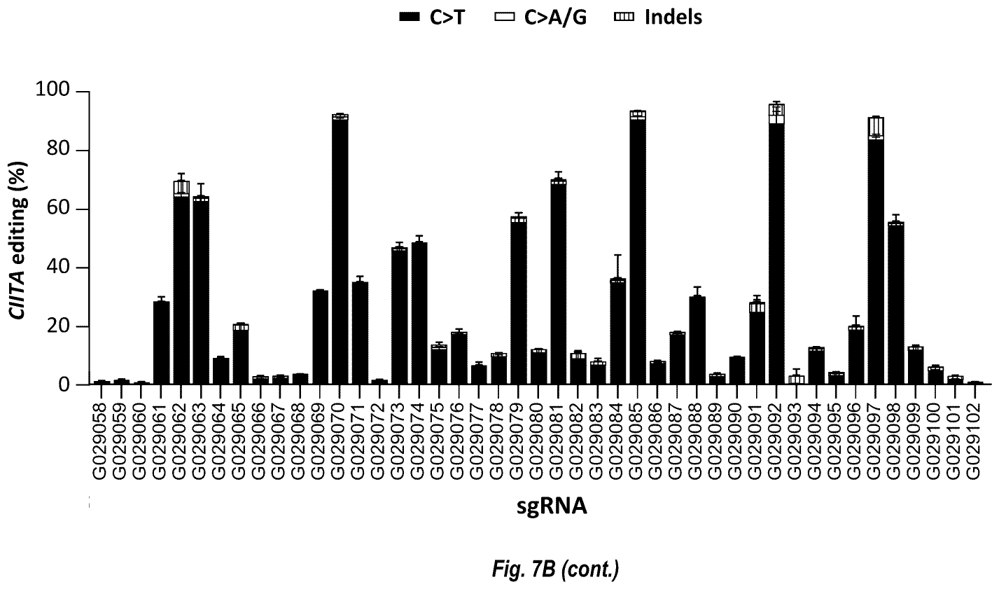

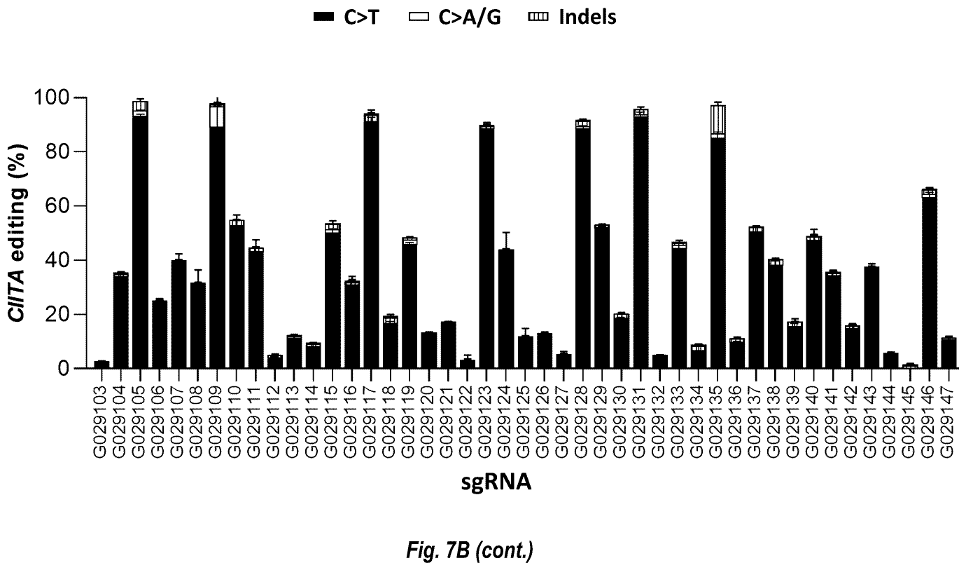

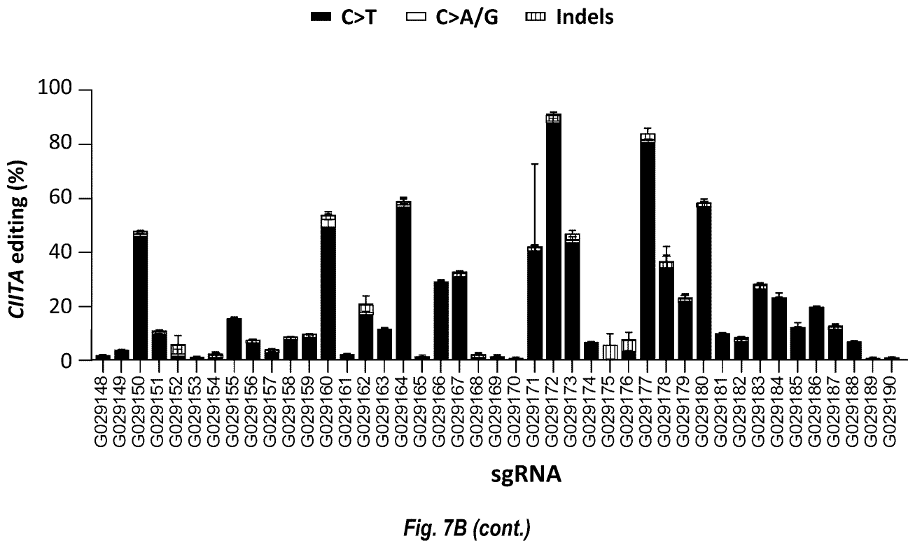

Table 14 and FIG. 7A show the mean percentage of CD3 T cells with loss of the HLA-DP, DQ, DR expression. Percent editing at the CIITA locus is shown in Table 14 and FIG. 7B.

TABLE 14
Mean percentage of HLA II-DP, DR, DQ negative cells; Mean percent
editing of the CIITA locus as a percentage of total NGS reads

| | % HLA | | | | | | | | | |
|---|---|---|---|---|---|---|---|---|---|---|
| | DP, DR, DQ -ve | | | % C to T | | % C to A/G | | % Indels | | |
| Guide ID | Mean | SD | N | Mean | SD | Mean | SD | Mean | SD | N |
| G029013 | 34.40 | 4.95 | 2 | 18.75 | 1.90 | 0.60 | 0.11 | 0.13 | 0.07 | 3 |
| G029014 | 54.60 | 5.80 | 2 | 96.18 | 0.32 | 1.71 | 0.05 | 0.78 | 0.15 | 3 |
| G029015 | 44.85 | 0.07 | 2 | 88.66 | 1.75 | 1.40 | 0.08 | 0.42 | 0.09 | 3 |
| G029016 | 37.05 | 7.99 | 2 | 3.20 | 0.50 | 0.23 | 0.17 | 0.02 | 0.02 | 3 |
| G029017 | 41.75 | 12.66 | 2 | 90.90 | 1.36 | 0.55 | 0.01 | 7.28 | 1.08 | 3 |
| G029018 | 45.05 | 4.74 | 2 | 46.49 | 0.66 | 0.41 | 0.09 | 3.13 | 0.17 | 3 |
| G029019 | 44.70 | 4.81 | 2 | 59.06 | 0.13 | 0.42 | 0.04 | 0.30 | 0.05 | 3 |
| G029020 | 37.85 | 3.46 | 2 | 40.66 | 0.14 | 0.52 | 0.15 | 0.31 | 0.04 | 3 |
| G029021 | 31.35 | 1.91 | 2 | 31.25 | 1.13 | 0.38 | 0.06 | 0.17 | 0.04 | 3 |
| G029022 | 38.60 | 0.42 | 2 | 5.70 | 1.01 | 0.44 | 0.36 | 0.21 | 0.27 | 3 |
| G029023 | 38.90 | 3.68 | 2 | 14.55 | 0.94 | 0.77 | 0.11 | 0.03 | 0.04 | 3 |
| G029024 | 29.75 | 4.17 | 2 | 12.24 | 0.41 | 0.23 | 0.04 | 0.06 | 0.03 | 3 |
| G029025 | 41.05 | 5.30 | 2 | 12.60 | 0.73 | 0.32 | 0.06 | 0.35 | 0.15 | 3 |
| G029026 | 49.35 | 1.34 | 2 | 5.75 | 0.66 | 0.24 | 0.06 | 0.04 | 0.03 | 3 |
| G029027 | 54.25 | 2.90 | 2 | 1.45 | 0.07 | 0.30 | 0.06 | 0.75 | 0.14 | 3 |
| G029028 | 50.50 | 0.71 | 2 | 68.54 | 3.78 | 2.29 | 0.09 | 6.12 | 0.59 | 3 |
| G029029 | 88.00 | 2.69 | 2 | 78.07 | 3.53 | 0.97 | 0.36 | 0.18 | 0.10 | 3 |
| G029030 | 77.85 | 1.48 | 2 | 87.88 | 0.43 | 0.57 | 0.23 | 0.60 | 0.03 | 3 |
| G029031 | 62.70 | 0.42 | 2 | 75.45 | 1.67 | 0.15 | 0.06 | 0.05 | 0.00 | 3 |
| G029032 | 49.60 | 2.12 | 2 | 2.41 | 0.30 | 0.17 | 0.12 | 0.17 | 0.23 | 3 |
| G029033 | 69.60 | 1.84 | 2 | 58.57 | 3.90 | 0.49 | 0.14 | 1.09 | 0.58 | 3 |
| G029034 | 50.90 | 6.08 | 1 | 16.00 | 2.53 | 0.38 | 0.28 | 0.10 | 0.06 | 3 |
| G029035 | 75.00 | 8.91 | 2 | 69.61 | 3.67 | 0.40 | 0.15 | 1.75 | 0.31 | 3 |
| G029036 | 71.65 | 14.78 | 2 | 0.00 | 0.00 | 0.00 | 0.00 | 0.00 | 0.00 | 3 |
| G029037 | 41.85 | 8.27 | 2 | 33.36 | 3.21 | 0.58 | 0.18 | 0.93 | 0.12 | 3 |
| G029038 | 56.00 | 2.55 | 2 | 6.63 | 3.25 | 0.31 | 0.13 | 0.01 | 0.02 | 3 |
| G029039 | 50.15 | 1.20 | 2 | 12.85 | 18.84 | 1.01 | 1.16 | 0.00 | 0.00 | 3 |
| G029040 | 55.50 | 0.57 | 2 | 36.13 | 12.71 | 0.30 | 0.24 | 0.01 | 0.02 | 3 |
| G029041 | 70.00 | 2.97 | 2 | 29.51 | 1.69 | 0.51 | 0.16 | 0.11 | 0.02 | 3 |
| G029042 | 52.10 | 0.85 | 2 | 14.95 | 2.44 | 0.45 | 0.04 | 0.03 | 0.00 | 3 |
| G029043 | 52.65 | 4.45 | 2 | 8.31 | 0.36 | 0.43 | 0.18 | 0.05 | 0.02 | 3 |
| G029044 | 54.40 | 1.13 | 2 | 3.50 | 0.04 | 0.47 | 0.13 | 0.07 | 0.05 | 3 |
| G029045 | 48.30 | 1.56 | 2 | 5.11 | 0.72 | 0.24 | 0.09 | 0.04 | 0.03 | 3 |
| G029046 | 60.60 | 2.83 | 2 | 85.08 | 2.95 | 1.26 | 0.24 | 0.58 | 0.11 | 3 |
| G029047 | 48.00 | 5.80 | 2 | 91.28 | 2.37 | 1.44 | 0.43 | 2.26 | 0.66 | 3 |
| G029048 | 56.35 | 3.32 | 2 | 77.44 | 0.57 | 1.31 | 0.68 | 1.76 | 0.63 | 3 |
| G029049 | 41.15 | 8.56 | 2 | 7.42 | 0.23 | 0.09 | 0.04 | 1.55 | 2.65 | 3 |
| G029050 | 59.15 | 4.74 | 2 | 19.53 | 0.87 | 0.17 | 0.07 | 0.02 | 0.02 | 3 |
| G029051 | 51.60 | 5.66 | 2 | 9.63 | 0.80 | 0.15 | 0.05 | 0.07 | 0.03 | 3 |
| G029052 | 52.60 | 1.98 | 2 | 5.81 | 0.21 | 0.29 | 0.04 | 0.20 | 0.06 | 3 |
| G029053 | 49.25 | 1.48 | 2 | 0.37 | 0.07 | 0.20 | 0.06 | 0.16 | 0.23 | 3 |
| G029054 | 47.60 | 11.03 | 2 | 3.23 | 0.11 | 0.14 | 0.03 | 0.04 | 0.04 | 3 |
| G029055 | 50.10 | 8.77 | 2 | 2.06 | 0.27 | 0.10 | 0.06 | 0.02 | 0.01 | 3 |
| G029056 | 62.45 | 2.76 | 2 | 79.87 | 3.36 | 0.79 | 0.08 | 1.87 | 0.15 | 3 |
| G029057 | 53.20 | 3.82 | 2 | 4.50 | 0.24 | 0.26 | 0.12 | 0.03 | 0.01 | 3 |
| G029058 | 51.95 | 5.16 | 2 | 0.91 | 0.15 | 0.12 | 0.03 | 0.26 | 0.18 | 3 |
| G029059 | 52.55 | 6.29 | 2 | 1.25 | 0.11 | 0.21 | 0.06 | 0.29 | 0.23 | 3 |
| G029060 | 43.25 | 3.46 | 2 | 0.45 | 0.11 | 0.18 | 0.10 | 0.22 | 0.17 | 3 |
| G029061 | 41.30 | 8.63 | 1 | 27.96 | 2.63 | 0.33 | 0.15 | 0.55 | 0.11 | 3 |
| G029062 | 57.55 | 0.21 | 2 | 65.48 | 5.49 | 1.08 | 0.15 | 4.29 | 2.51 | 3 |
| G029063 | 72.90 | 0.71 | 2 | 63.82 | 6.02 | 1.43 | 0.25 | 0.19 | 0.07 | 3 |
| G029064 | 50.15 | 4.31 | 2 | 8.25 | 1.52 | 0.38 | 0.07 | 0.60 | 0.30 | 3 |
| G029065 | 45.90 | 2.12 | 2 | 19.00 | 1.71 | 1.61 | 0.46 | 0.41 | 0.37 | 3 |
| G029066 | 43.80 | 7.07 | 2 | 1.93 | 0.50 | 0.95 | 0.24 | 0.08 | 0.03 | 3 |
| G029067 | 45.75 | 0.78 | 2 | 2.32 | 0.48 | 0.78 | 0.27 | 0.06 | 0.02 | 3 |
| G029068 | 44.55 | 0.35 | 2 | 3.58 | 0.11 | 0.14 | 0.05 | 0.04 | 0.03 | 3 |
| G029069 | 52.75 | 3.46 | 2 | 32.35 | 0.63 | 0.35 | 0.02 | 0.10 | 0.05 | 3 |
| G029070 | 73.30 | 4.38 | 2 | 92.11 | 0.53 | 1.28 | 0.22 | 0.67 | 0.14 | 3 |
| G029071 | 50.65 | 1.06 | 2 | 35.16 | 2.51 | 0.29 | 0.08 | 0.19 | 0.10 | 3 |
| G029072 | 42.35 | 2.33 | 2 | 1.47 | 0.07 | 0.27 | 0.03 | 0.05 | 0.04 | 3 |
| G029073 | 41.30 | 9.48 | 2 | 46.89 | 2.53 | 0.66 | 0.23 | 0.21 | 0.15 | 3 |
| G029074 | 52.20 | 0.14 | 2 | 48.77 | 3.02 | 0.61 | 0.04 | 0.19 | 0.08 | 3 |
| G029075 | 49.30 | 1.13 | 2 | 12.47 | 1.16 | 0.69 | 0.19 | 0.76 | 0.82 | 3 |
| G029076 | 43.45 | 3.04 | 2 | 17.10 | 0.40 | 0.47 | 0.05 | 0.79 | 0.97 | 3 |
| G029077 | 48.20 | 2.55 | 2 | 6.55 | 1.34 | 0.16 | 0.12 | 0.14 | 0.03 | 3 |
| G029078 | 43.30 | 0.42 | 2 | 9.78 | 0.71 | 1.18 | 0.12 | 0.07 | 0.07 | 3 |
| G029079 | 58.65 | 0.35 | 2 | 56.55 | 3.15 | 1.49 | 0.10 | 0.41 | 0.04 | 3 |
| G029080 | 50.35 | 0.21 | 2 | 11.06 | 0.91 | 1.24 | 0.14 | 0.07 | 0.03 | 3 |
| G029081 | 85.40 | 1.84 | 2 | 69.63 | 4.27 | 1.26 | 0.21 | 0.57 | 0.31 | 3 |
| G029082 | 51.85 | 4.17 | 2 | 9.09 | 2.32 | 1.87 | 0.85 | 0.04 | 0.04 | 3 |
| G029083 | 46.55 | 0.35 | 2 | 6.81 | 2.37 | 1.21 | 0.07 | 0.03 | 0.03 | 3 |
| G029084 | 46.60 | 12.16 | 2 | 35.64 | 9.40 | 0.94 | 0.55 | 0.40 | 0.14 | 3 |
| G029085 | 38.40 | 7.50 | 2 | 92.18 | 1.18 | 1.09 | 0.03 | 1.98 | 0.05 | 3 |
| G029086 | 44.85 | 2.47 | 2 | 7.25 | 0.44 | 0.34 | 0.08 | 0.73 | 0.16 | 3 |
| G029087 | 47.30 | 0.28 | 2 | 17.14 | 1.34 | 0.43 | 0.07 | 0.75 | 0.17 | 3 |
| G029088 | 49.85 | 4.17 | 2 | 30.15 | 3.86 | 0.20 | 0.34 | 0.22 | 0.38 | 3 |
| G029089 | 45.00 | 0.28 | 2 | 2.98 | 0.25 | 0.66 | 0.38 | 0.10 | 0.05 | 3 |
| G029090 | 39.10 | 2.26 | 2 | 8.97 | 0.53 | 0.39 | 0.20 | 0.34 | 0.13 | 3 |
| G029091 | 40.05 | 3.18 | 2 | 25.28 | 4.23 | 3.02 | 2.63 | 0.31 | 0.28 | 3 |
| G029092 | 49.50 | 0.99 | 2 | 90.70 | 4.31 | 3.27 | 2.57 | 3.66 | 0.69 | 3 |
| G029093 | 46.70 | 7.07 | 2 | 0.39 | 0.05 | 2.79 | 2.28 | 0.08 | 0.07 | 3 |
| G029094 | 46.40 | 4.81 | 2 | 11.75 | 0.73 | 0.98 | 0.08 | 0.33 | 0.10 | 3 |
| G029095 | 42.75 | 7.57 | 2 | 3.37 | 0.46 | 0.89 | 0.17 | 0.11 | 0.05 | 3 |
| G029096 | 33.95 | 4.60 | 2 | 19.19 | 4.61 | 0.94 | 0.18 | 0.33 | 0.19 | 3 |
| G029097 | 33.90 | 1.70 | 2 | 85.22 | 0.86 | 1.66 | 0.16 | 6.17 | 0.23 | 3 |
| G029098 | 45.70 | 1.41 | 2 | 55.36 | 3.68 | 0.96 | 0.14 | 0.35 | 0.16 | 3 |
| G029099 | 42.75 | 0.64 | 2 | 11.94 | 1.85 | 1.17 | 0.08 | 0.14 | 0.06 | 3 |
| G029100 | 34.55 | 0.49 | 2 | 4.72 | 0.45 | 1.52 | 0.56 | 0.13 | 0.15 | 3 |
| G029101 | 40.95 | 0.21 | 2 | 1.72 | 0.44 | 1.16 | 0.39 | 0.07 | 0.06 | 3 |
| G029102 | 35.40 | 1.98 | 2 | 0.44 | 0.13 | 0.50 | 0.05 | 0.08 | 0.05 | 3 |
| G029103 | 32.20 | 0.99 | 2 | 2.30 | 0.43 | 0.35 | 0.24 | 0.10 | 0.13 | 4 |
| G029104 | 50.40 | 5.52 | 2 | 34.26 | 1.13 | 0.94 | 0.21 | 0.38 | 0.18 | 4 |
| G029105 | 38.05 | 1.34 | 2 | 93.46 | 0.85 | 1.80 | 0.13 | 3.54 | 0.79 | 4 |
| G029106 | 43.05 | 3.18 | 2 | 24.88 | 0.99 | 0.31 | 0.07 | 0.09 | 0.06 | 4 |
| G029107 | 49.40 | 2.97 | 2 | 39.56 | 2.80 | 0.35 | 0.05 | 0.28 | 0.02 | 4 |
| G029108 | 39.05 | 9.69 | 2 | 31.33 | 5.11 | 0.27 | 0.02 | 0.15 | 0.05 | 4 |
| G029109 | 71.15 | 0.92 | 2 | 89.12 | 9.14 | 8.05 | 8.87 | 0.79 | 0.20 | 4 |
| G029110 | 37.25 | 0.78 | 2 | 53.06 | 3.72 | 1.41 | 0.60 | 0.45 | 0.15 | 4 |
| G029111 | 35.20 | 2.40 | 2 | 43.21 | 4.33 | 1.02 | 0.22 | 0.47 | 0.25 | 4 |
| G029112 | 41.90 | 0.42 | 2 | 4.38 | 0.37 | 0.67 | 0.21 | 0.22 | 0.20 | 4 |
| G029113 | 41.30 | 9.19 | 2 | 11.35 | 0.91 | 0.66 | 0.04 | 0.45 | 0.16 | 4 |
| G029114 | 34.00 | 3.96 | 2 | 8.69 | 0.44 | 0.63 | 0.08 | 0.27 | 0.03 | 4 |
| G029115 | 45.35 | 11.67 | 2 | 50.53 | 1.40 | 0.99 | 0.19 | 2.22 | 0.77 | 4 |
| G029116 | 59.55 | 4.88 | 2 | 31.29 | 2.75 | 0.81 | 0.09 | 0.49 | 0.24 | 4 |
| G029117 | 81.45 | 0.35 | 2 | 91.14 | 1.77 | 2.68 | 1.60 | 0.41 | 0.10 | 4 |
| G029118 | 51.90 | 1.27 | 2 | 16.93 | 0.68 | 1.79 | 0.61 | 0.89 | 0.47 | 4 |
| G029119 | 63.15 | 5.30 | 2 | 46.34 | 0.58 | 1.54 | 0.39 | 0.65 | 0.16 | 4 |
| G029120 | 55.30 | 2.69 | 2 | 12.79 | 0.80 | 0.47 | 0.14 | 0.13 | 0.06 | 4 |
| G029121 | 56.15 | 3.32 | 2 | 16.62 | 0.64 | 0.62 | 0.17 | 0.07 | 0.05 | 4 |
| G029122 | 55.30 | 8.34 | 2 | 3.08 | 1.96 | 0.20 | 0.27 | 0.10 | 0.17 | 4 |
| G029123 | 90.80 | 0.99 | 2 | 89.28 | 0.71 | 0.63 | 0.94 | 0.17 | 0.22 | 4 |
| G029124 | 65.90 | 3.39 | 2 | 43.34 | 6.94 | 0.19 | 0.13 | 0.42 | 0.28 | 4 |
| G029125 | 54.35 | 4.31 | 2 | 11.68 | 3.17 | 0.23 | 0.27 | 0.06 | 0.07 | 4 |
| G029126 | 37.75 | 0.49 | 2 | 12.13 | 1.22 | 0.58 | 0.19 | 0.47 | 0.40 | 4 |
| G029127 | 45.20 | 7.07 | 2 | 5.05 | 1.26 | 0.28 | 0.32 | 0.06 | 0.08 | 4 |
| G029128 | 81.55 | 2.05 | 2 | 88.85 | 2.04 | 0.82 | 0.29 | 2.03 | 0.33 | 4 |
| G029129 | 75.35 | 5.02 | 2 | 51.88 | 0.94 | 0.49 | 0.04 | 0.86 | 0.13 | 4 |
| G029130 | 58.20 | 8.77 | 2 | 18.50 | 1.66 | 0.40 | 0.15 | 1.43 | 0.40 | 4 |
| G029131 | 93.15 | 1.91 | 2 | 93.47 | 1.53 | 0.78 | 0.37 | 1.63 | 0.67 | 4 |
| G029132 | 53.50 | 8.20 | 2 | 4.52 | 0.15 | 0.53 | 0.11 | 0.05 | 0.03 | 4 |
| G029133 | 63.55 | 1.91 | 2 | 44.52 | 1.48 | 1.33 | 0.30 | 0.99 | 0.51 | 4 |
| G029134 | 47.70 | 1.98 | 2 | 7.17 | 1.32 | 1.32 | 0.54 | 0.34 | 0.31 | 4 |
| G029135 | 43.25 | 61.16 | 2 | 85.48 | 1.21 | 1.52 | 0.31 | 10.28 | 1.03 | 4 |
| G029136 | 52.75 | 2.47 | 2 | 10.14 | 0.23 | 1.00 | 0.46 | 0.13 | 0.06 | 4 |
| G029137 | 65.90 | 0.57 | 2 | 50.89 | 1.82 | 1.30 | 0.57 | 0.27 | 0.07 | 4 |
| G029138 | 57.60 | 6.79 | 2 | 38.26 | 1.45 | 2.03 | 0.49 | 0.25 | 0.05 | 4 |
| G029139 | 48.30 | 1.27 | 2 | 16.01 | 0.82 | 1.37 | 1.10 | 0.06 | 0.06 | 4 |
| G029140 | 75.15 | 0.07 | 2 | 47.78 | 3.71 | 1.10 | 0.64 | 0.17 | 0.06 | 4 |
| G029141 | 69.90 | 3.82 | 2 | 34.65 | 1.70 | 0.93 | 0.61 | 0.32 | 0.39 | 4 |
| G029142 | 60.95 | 3.75 | 2 | 15.07 | 1.14 | 0.89 | 0.68 | 0.06 | 0.06 | 4 |
| G029143 | 67.65 | 1.06 | 2 | 36.84 | 1.93 | 0.63 | 0.32 | 0.18 | 0.09 | 4 |
| G029144 | 56.70 | 2.55 | 2 | 5.40 | 0.54 | 0.44 | 0.31 | 0.04 | 0.02 | 4 |
| G029145 | 54.40 | 9.48 | 2 | 0.78 | 0.04 | 0.68 | 0.53 | 0.18 | 0.17 | 4 |
| G029146 | 63.20 | 8.63 | 2 | 63.58 | 3.19 | 0.93 | 0.80 | 1.93 | 0.28 | 4 |
| G029147 | 56.05 | 1.20 | 2 | 10.86 | 0.91 | 0.66 | 0.41 | 0.05 | 0.04 | 4 |
| G029148 | 51.95 | 0.49 | 2 | 1.50 | 0.36 | 0.35 | 0.20 | 0.04 | 0.03 | 4 |
| G029149 | 59.30 | 2.69 | 2 | 3.23 | 0.18 | 0.49 | 0.15 | 0.14 | 0.08 | 4 |
| G029150 | 49.40 | 1.70 | 2 | 46.90 | 1.43 | 1.09 | 0.23 | 0.89 | 0.15 | 4 |
| G029151 | 52.95 | 9.83 | 2 | 9.70 | 0.57 | 0.59 | 0.42 | 0.89 | 0.12 | 4 |
| G029152 | 56.90 | 3.11 | 2 | 1.80 | 0.23 | 1.06 | 1.52 | 3.20 | 3.20 | 4 |
| G029153 | 52.90 | 4.95 | 2 | 0.23 | 0.10 | 0.65 | 0.27 | 0.29 | 0.20 | 4 |
| G029154 | 54.45 | 4.88 | 2 | 1.15 | 0.38 | 0.49 | 0.12 | 0.85 | 0.61 | 4 |
| G029155 | 56.70 | 0.28 | 2 | 14.72 | 1.08 | 0.67 | 0.12 | 0.44 | 0.42 | 4 |
| G029156 | 53.75 | 0.35 | 2 | 6.87 | 0.62 | 0.73 | 0.14 | 0.13 | 0.11 | 4 |
| G029157 | 56.25 | .06 | 2 | 3.13 | 0.37 | 0.78 | 0.10 | 0.20 | 0.09 | 4 |
| G029158 | 51.60 | 3.96 | 2 | 7.70 | 0.81 | 1.02 | 0.02 | 0.10 | 0.03 | 4 |
| G029159 | 56.45 | 1.20 | 2 | 8.61 | 0.59 | 1.16 | 0.19 | 0.18 | 0.03 | 4 |
| G029160 | 72.15 | 5.02 | 2 | 50.61 | 2.24 | 3.06 | 2.44 | 1.28 | 0.34 | 4 |
| G029161 | 59.45 | 2.62 | 2 | 1.81 | 0.22 | 0.39 | 0.18 | 0.07 | 0.04 | 4 |
| G029162 | 50.50 | 2.12 | 2 | 17.38 | 1.22 | 0.93 | 0.50 | 3.06 | 2.91 | 4 |
| G029163 | 46.55 | 9.97 | 2 | 11.23 | 0.99 | 0.57 | 0.18 | 0.04 | 0.03 | 4 |
| G029164 | 67.15 | 2.33 | 2 | 58.31 | 3.24 | 0.96 | 0.32 | 0.92 | 0.73 | 4 |
| G029165 | 53.65 | 4.17 | 2 | 0.71 | 0.24 | 0.19 | 0.14 | 0.42 | 0.49 | 4 |
| G029166 | 53.45 | 0.35 | 2 | 29.13 | 1.23 | 0.48 | 0.08 | 0.27 | 0.20 | 4 |
| G029167 | 66.60 | 1.84 | 2 | 32.25 | 1.39 | 1.11 | 0.13 | 0.15 | 0.23 | 4 |
| G029168 | 50.20 | 6.93 | 2 | 1.25 | 0.27 | 0.93 | 0.53 | 0.13 | 0.20 | 4 |
| G029169 | 49.80 | 6.51 | 2 | 0.89 | 1.05 | 0.47 | 0.55 | 0.05 | 0.07 | 4 |
| G029170 | 49.35 | 5.4 | 2 | 0.00 | 0.00 | 0.00 | 0.00 | 0.00 | 0.00 | |
| G029171 | 84.10 | 0.00 | 2 | 0.00 | 0.00 | 0.00 | 0.00 | 0.00 | 0.00 | |
| G029172 | 95.20 | 0.42 | 2 | 90.18 | 1.06 | 1.15 | 0.09 | 1.95 | 0.62 | 4 |
| G029173 | 72.50 | 4.95 | 2 | 45.16 | 1.85 | 0.93 | 0.03 | 1.85 | 1.06 | 4 |
| G029174 | 59.55 | 5.59 | 2 | 6.11 | 0.81 | 0.58 | 0.06 | 0.10 | 0.05 | 4 |
| G029175 | 49.80 | 3.25 | 2 | 0.28 | 0.10 | 0.29 | 0.19 | 5.18 | 4.22 | 4 |
| G029176 | 48.35 | 4.88 | 2 | 3.15 | 0.46 | 0.23 | 0.16 | 4.47 | 2.55 | 4 |
| G029177 | 79.80 | 1.41 | 2 | 83.07 | 2.06 | 0.76 | 0.11 | 2.00 | 1.95 | 4 |
| G029178 | 53.80 | 0.85 | 2 | 34.52 | 8.46 | 0.41 | 0.17 | 2.52 | 2.17 | 4 |
| G029179 | 51.45 | 7.14 | 2 | 22.28 | 2.43 | 0.24 | 0.04 | 1.25 | 1.31 | 4 |
| G029180 | 51.75 | 4.88 | 2 | 58.42 | 2.55 | 1.21 | 0.13 | 0.21 | 0.05 | 4 |
| G029181 | 51.15 | 4.88 | 2 | 9.33 | 0.23 | 0.67 | 0.30 | 0.14 | 0.14 | 4 |
| G029182 | 49.95 | 2.76 | 2 | 7.53 | 0.57 | 0.83 | 0.09 | 0.29 | 0.24 | 4 |
| G029183 | 49.20 | 2.12 | 2 | 27.27 | 1.48 | 1.14 | 0.21 | 0.45 | 0.36 | 4 |
| G029184 | 36.70 | 2.83 | 2 | 23.25 | 2.15 | 0.36 | 0.20 | 0.11 | 0.05 | 4 |
| G029185 | 42.00 | 4.81 | 2 | 11.98 | 2.17 | 0.33 | 0.14 | 0.11 | 0.09 | 4 |
| G029186 | 55.05 | 2.76 | 2 | 19.73 | 0.53 | 0.41 | 0.18 | 0.15 | 0.05 | 4 |
| G029187 | 59.60 | 1.56 | 2 | 12.22 | 1.40 | 0.79 | 0.13 | 0.04 | 0.04 | 4 |
| G029188 | 57.50 | 7.21 | 2 | 6.53 | 0.36 | 0.41 | 0.35 | 0.20 | 0.16 | 4 |
| G029189 | 49.20 | 4.38 | 2 | 0.13 | 0.04 | 0.65 | 0.22 | 0.04 | 0.05 | 4 |
| G029190 | 44.05 | 2.76 | 2 | 0.40 | 0.09 | 0.54 | 0.19 | 0.04 | 0.04 | 4 |

8.2. Screening of CIITA Guide RNAs with Nme2 Cas9

Additional sgRNAs targeting CIITA were screened for efficacy in T cells by assessing loss of HLA-DP/DQ/DR cell surface expression by flow cytometry and editing by NGS, following CIITA editing by mRNA delivery.

8.2.1 T Cell Preparation and Editing with RNA Electroporation

T cells were prepared as described in Example 1 using 2.5% o human AB serum (GeminiBio, Cat. 100-512) in the T Cell growth media. T cells were electroporated with sgRNA targeting the CIITA gene using mRNA electroporation as described in Example 1 except for the following differences. This study used mRNA encoding Nme2 Cas9 (SEQ ID No: 826) instead of mRNAs encoding Nme2 BC22n base editor and UGI, respectively.

Cas9 electroporation mix was prepared with 1×10⁵ T cells, 30 ng/L of Nme2 Cas9 mRNA and 40 pmols of sgRNA in a final volume of 20 μL of P3 electroporation buffer.

### 8.2.2 Flow Cytometry and NGS Sequencing

On day 7 post-electroporation, edited T cell samples were collected and subjected to PCR and NGS analysis as described in Example 1. On day 13 post-editing, cells were phenotyped by flow cytometry to determine HLA II-DR, DP, DQ protein expression. Briefly, T cells were incubated for 30 min at 4° C. with a mixture of antibodies against CD3 (BioLegend, Cat. No. 317338), CD4 (BioLegend, Cat. No. 317434), CD8 (BioLegend, Cat. No. 301046), Viakrome (Beckman Coulter, Cat. No. C36628) diluted at 1:100, and HLA II-DR, DP, DQ (BioLegend, Cat. No. 361706) diluted at 1:50 in cell staining buffer (BioLegend, Cat. No. 420201) to determine HLA-DP, DQ, DR protein expression. T cells were gated based on size, shape, viability, CD3, CD8, and HLA II-DR, DP, DQ, expression.

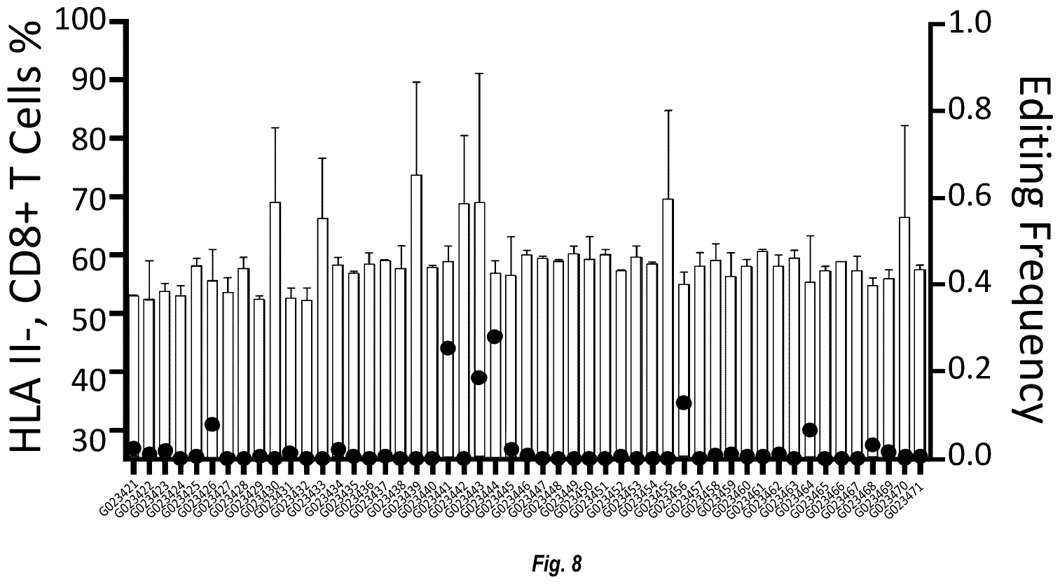

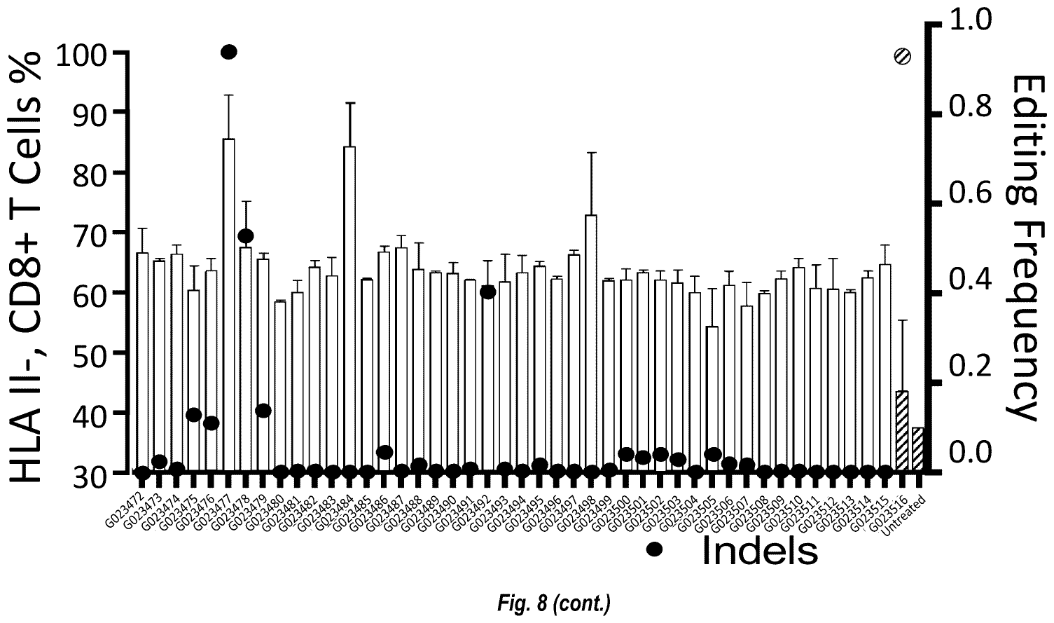

Table 15 shows the percentage of T cells with loss of HLA II and mean percent editing of the CIITA gene. FIG. 8 shows percentage of HLA II-DR, DP, DQ negative cells edited with Nme2 Cas9 on the left vertical axis and mean percent editing on the right vertical axis.

TABLE 15
Mean percentage of HLA II-DP, DR, DQ negative cells; Mean percent
indels oatthe CIITA locus as a percentage of total NGS reads

| | | CD8+, HLA-DP, |
|---|---|---|
| Guide | % INdels | DQ, DR− |

| ID | Mean | SD | Mean | SD | N |
|---|---|---|---|---|---|
| Untreated | | | 40.25 | 0.05 | 2 |
| G023421 | 2.55 | 0.25 | 45.2 | 8.1 | 2 |
| G023422 | 1.1 | 0 | 46.4 | 5.1 | 2 |
| G023423 | 1.7 | 0.4 | 47.7 | 1.7 | 2 |
| G023424 | 0.35 | 0.05 | 44.45 | 1.25 | 2 |
| G023425 | 0.65 | 0.55 | 46.25 | 2.95 | 2 |
| G023426 | 7.7 | 2.1 | 50.15 | 1.55 | 2 |
| G023427 | 0.5 | 0.1 | 46.15 | 4.35 | 2 |
| G023428 | 0.15 | 0.05 | 50.4 | 0.4 | 2 |
| G023429 | 0.9 | 0.1 | 48.15 | 2.35 | 2 |
| G023430 | 0.1 | 0 | 50.3 | 1.5 | 2 |
| G023431 | 1.55 | 0.35 | 50.8 | 1.6 | 2 |
| G023432 | 0.15 | 0.05 | 52.45 | 0.85 | 2 |
| G023433 | 0.15 | 0.05 | 46.25 | 0.65 | 2 |
| G023434 | 2.1 | 0.2 | 49.85 | 0.05 | 2 |
| G023435 | 0.65 | 0.05 | 52.05 | 0.85 | 2 |
| G023436 | 0.35 | 0.05 | 53.5 | 1.4 | 2 |
| G023437 | 0.9 | 0.1 | 52.1 | 0.1 | 2 |
| G023438 | 0.15 | 0.15 | 53.15 | 1.35 | 2 |
| G023439 | 0.1 | 0 | 51.05 | 0.25 | 2 |
| G023440 | 0.35 | 0.15 | 54.35 | 1.05 | 2 |
| G023441 | 25.4 | 1.1 | 59.7 | 4.3 | 2 |
| G023442 | 0.2 | 0 | 57.75 | 1.55 | 2 |
| G023443 | 18.65 | 0.25 | 69.15 | 15.05 | 2 |
| G023444 | 28.15 | 6.05 | 58.65 | 1.85 | 2 |
| G023445 | 2.45 | 0.35 | 55.25 | 0.15 | 2 |
| G023446 | 0.9 | 0.3 | 49.15 | 0.55 | 2 |
| G023447 | 0.35 | 0.15 | 54.45 | 3.75 | 2 |
| G023448 | 0.2 | 0 | 51.5 | 0.6 | 2 |
| G023449 | 0 | 0 | 53.6 | 3 | 2 |
| G023450 | 0.1 | 0 | 54 | 2.3 | 2 |
| G023451 | 0.15 | 0.05 | 51.05 | 2.75 | 2 |
| G023452 | 0.55 | 0.15 | 52.75 | 2.45 | 2 |
| G023453 | 0.1 | 0 | 52.85 | 3.55 | 2 |
| G023454 | 0.35 | 0.15 | 55.65 | 4.35 | 2 |
| G023455 | 0.25 | 0.05 | 53.3 | 4.2 | 2 |
| G023456 | 12.8 | 2 | 56 | 3 | 2 |
| G023457 | 0.35 | 0.05 | 55.85 | 3.75 | 2 |
| G023458 | 1 | 0.3 | 52 | 3.2 | 2 |
| G023459 | 1.3 | 0.4 | 51.15 | 3.05 | 2 |
| G023460 | 0.45 | 0.05 | 53.75 | 1.35 | 2 |
| G023461 | 0.45 | 0.05 | 53.25 | 1.65 | 2 |
| G023462 | 1.05 | 0.15 | 52.2 | 4 | 2 |
| G023463 | 0.2 | 0 | 51.15 | 3.95 | 2 |
| G023464 | 6.6 | 1 | 54.1 | 1 | 2 |
| G023465 | 0.05 | 0.05 | 52.45 | 0.45 | 2 |
| G023466 | 0.2 | 0.2 | 51.2 | 2.7 | 2 |
| G023467 | 0.2 | 0 | 54.75 | 2.45 | 2 |
| G023468 | 3.25 | 0.15 | 58.3 | 3.9 | 2 |
| G023469 | 1.6 | 0.1 | 56.85 | 4.55 | 2 |
| G023470 | 0.1 | 0 | 54.25 | 2.95 | 2 |
| G023471 | 0.55 | 0.25 | 53.65 | 4.05 | 2 |
| G023472 | 0.1 | 0 | 53.2 | 1.7 | 2 |
| G023473 | 2.5 | 1 | 57.2 | 2.3 | 2 |
| G023474 | 0.6 | 0.1 | 57.65 | 1.75 | 2 |
| G023475 | 12.75 | 1.05 | 58.65 | 2.25 | 2 |
| G023476 | 10.9 | 1.3 | 58.25 | 1.05 | 2 |
| G023477 | 93.8 | 1.1 | 80.45 | 3.95 | 2 |
| G023478 | 52.75 | 5.95 | 64.9 | 2.2 | 2 |
| G023479 | 13.75 | 2.05 | 58.85 | 0.25 | 2 |
| G023480 | 0.1 | 0 | 55.5 | 1.2 | 2 |
| G023481 | 0.2 | 0 | 54.8 | 1.4 | 2 |
| G023482 | 0.5 | 0.4 | 52.8 | 2.1 | 2 |
| G023483 | 0.1 | 0 | 53.35 | 2.65 | 2 |
| G023484 | 0.1 | 0 | 59.7 | 8.4 | 2 |
| G023485 | 0.25 | 0.05 | 50.2 | 0.2 | 2 |
| G023486 | 4.5 | 0.2 | 52.45 | 0.65 | 2 |
| G023487 | 0.4 | 0.1 | 52.85 | 3.35 | 2 |
| G023488 | 1.4 | 0.2 | 52 | 2.7 | 2 |
| G023489 | 0.15 | 0.05 | 52.85 | 2.35 | 2 |
| G023490 | 0.15 | 0.15 | 53.2 | 1.8 | 2 |
| G023491 | 1 | 0.2 | 53.45 | 0.95 | 2 |
| G023492 | 40.25 | 12.05 | 59.6 | 0.8 | 2 |
| G023493 | 0.8 | 0.4 | 56.7 | 0.9 | 2 |
| G023494 | 0.3 | 0.2 | 51.55 | 2.35 | 2 |
| G023495 | 1.5 | 1.3 | 53.25 | 1.05 | 2 |
| G023496 | 0.1 | 0 | 52.05 | 3.15 | 2 |
| G023497 | 0.05 | 0.05 | 52.85 | 0.35 | 2 |
| G023498 | 0.1 | 0 | 53 | 0.9 | 2 |
| G023499 | 0.4 | 0 | 53.65 | 4.05 | 2 |
| G023500 | 4.2 | 0.3 | 53.7 | 3 | 2 |
| G023501 | 3.1 | 0.1 | 51.95 | 2.35 | 2 |
| G023502 | 3.95 | 0.35 | 53.95 | 0.95 | 2 |
| G023503 | 2.95 | 0.05 | 53.5 | 3.5 | 2 |
| G023504 | 0.25 | 0.05 | 51.7 | 1.6 | 2 |
| G023505 | 4.1 | 0.1 | 52.9 | 1 | 2 |
| G023506 | 1.95 | 0.15 | 49.8 | 0.9 | 2 |
| G023507 | 1.3 | 1 | 53.2 | 3.4 | 2 |
| G023508 | 0.1 | 0 | 53.15 | 5.35 | 2 |
| G023509 | 0.15 | 0.05 | 51.85 | 0.85 | 2 |
| G023510 | 0.15 | 0.05 | 53.8 | 0.3 | 2 |
| G023511 | 0.25 | 0.05 | 51.75 | 0.25 | 2 |
| G023512 | 0.1 | 0 | 51.75 | 2.65 | 2 |
| G023513 | 0.1 | 0 | 53.85 | 2.35 | 2 |
| G023514 | 0.35 | 0.05 | 53.1 | 2.1 | 2 |
| G023515 | 0.1 | 0 | 51.95 | 0.95 | 2 |

### Example 9: Dose Response Curves (DRC) for Select CIITA Nme2 Guides

A titration experiment was conducted to evaluate the editing efficacy of select sgRNAs designed for the disruption of the CIITA gene by assessing loss of HLA DP, DQ, DR cell surface expression by flow cytometry and editing frequency at CIITA by NGS. HLA-DP, DR and DQ are cell surface proteins whose expression requires the transcription factor CIITA and thus their presence at the cell surface is a surrogate marker for CIITA protein expression.

An eight-point dose response curve was generated for each sgRNA by titrating each sgRNA with a fixed concentration of mRNA encoding Nme2 BC22n (SEQ ID NO:822) and mRNA encoding UGI (SEQ ID NO: 821) in T cells using electroporation. T cells were then expanded and phenotyped by flow cytometry to determine the editing efficiency of each sgRNA tested.

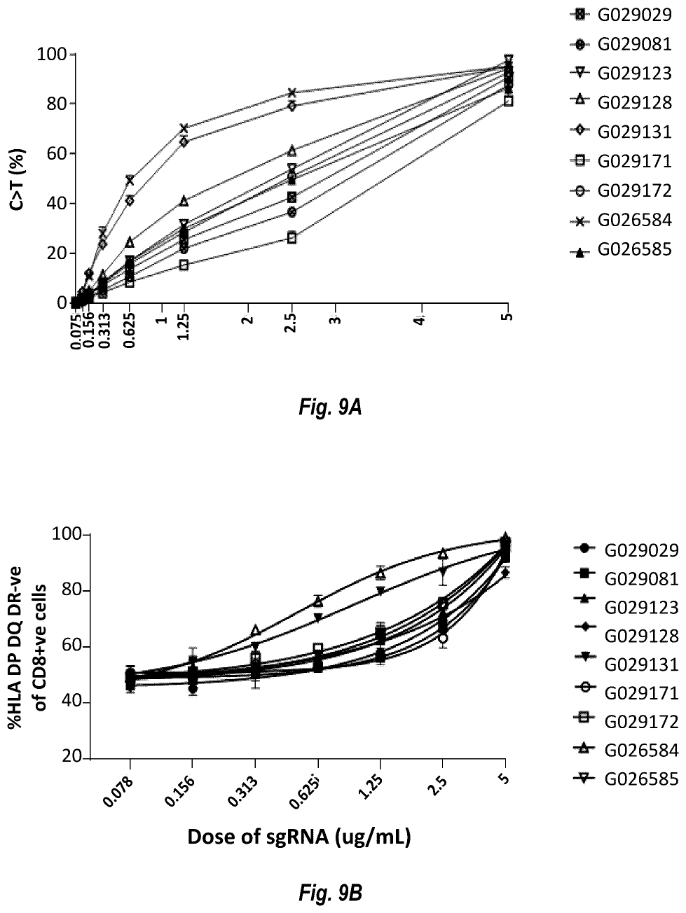

T cells were prepared single donor (no. 808) as described in Example 1. T cells were edited with serially diluted CIITA targeting sgRNAs (0, 0.94, 1.88, 3.75, 7.5, 15, 30, or 60 pmols) by mRNA electroporation as described in Example 1. On day 4 post-editing, T cell samples were collected and subjected to PCR and NGS analysis as described in Example 1. On day 7 post-editing, T cells were phenotyped by flow cytometry to determine HLA-DP, DQ, DR expression as described in Example 8. T cells were gated based on size, singularity, viability and CD8 positivity. Table 16 and FIG. 9A show the NGS editing results. Table 16 and FIG. 9B show the percentage of CD8+ T cells that are negative for HLA-DP, DQ, DR expression

TABLE 16

| | | | | | | | | | | % HLA II-DP, DQ, DR- of | | |
|---|---|---|---|---|---|---|---|---|---|---|---|---|
| sgRNA | C > T | | | C > A/G | | | Indels | | | CD8+ | | |
| (pmols) | Ave | SD | N | Ave | SD | N | Ave | SD | N | Ave | SD | N |

G029029

| | | | | | | | | | | | | |
|---|---|---|---|---|---|---|---|---|---|---|---|---|
| 60 | 90.74% | 1.38% | 2 | 1.62% | 0.27% | 2 | 0.43% | 0.08% | 2 | 91.95 | 1.77 | 2 |
| 30 | 42.49% | 0.45% | 2 | 1.70% | 0.22% | 2 | 0.15% | 0.04% | 2 | 69.45 | 1.20 | 2 |
| 15 | 25.50% | 2.00% | 2 | 1.64% | 0.17% | 2 | 0.11% | 0.05% | 2 | 58.1 | 3.25 | 2 |
| 7.5 | 13.63% | 0.29% | 2 | 1.96% | 0.12% | 2 | 0.05% | 0.00% | 2 | 52.7 | 0.99 | 2 |
| 3.75 | 7.08% | 0.37% | 2 | 1.58% | 0.16% | 2 | 0.07% | 0.00% | 2 | 50.35 | 2.33 | 2 |
| 1.88 | 3.08% | 0.13% | 2 | 1.49% | 0.20% | 2 | 0.04% | 0.00% | 2 | 45.2 | 2.40 | 2 |
| 0.94 | 1.46% | 0.34% | 2 | 1.42% | 0.19% | 2 | 0.03% | 0.01% | 2 | 47.15 | 1.48 | 2 |
| 0 | 0.04% | 0.00% | 2 | 2.51% | 1.11% | 2 | 0.03% | 0.02% | 2 | 45.3 | 4.81 | 2 |

G029081

| | | | | | | | | | | | | |
|---|---|---|---|---|---|---|---|---|---|---|---|---|
| 60 | 87.77% | 0.42% | 2 | 1.29% | 0.03% | 2 | 1.05% | 0.22% | 2 | 92.15 | 1.48 | 2 |
| 30 | 36.59% | 1.71% | 2 | 1.06% | 0.11% | 2 | 0.42% | 0.03% | 2 | 66.85 | 2.33 | 2 |
| 15 | 21.90% | 1.00% | 2 | 0.86% | 0.11% | 2 | 0.19% | 0.03% | 2 | 56.2 | 2.55 | 2 |
| 7.5 | 10.56% | 0.50% | 2 | 0.90% | 0.02% | 2 | 0.17% | 0.01% | 2 | 52.8 | 0.85 | 2 |
| 3.75 | 5.17% | 0.57% | 2 | 1.04% | 0.04% | 2 | 0.06% | 0.01% | 2 | 50.7 | 1.70 | 2 |
| 1.88 | 2.39% | 0.07% | 2 | 0.86% | 0.01% | 2 | 0.04% | 0.00% | 2 | 49.15 | 1.77 | 2 |
| 0.94 | 1.01% | 0.06% | 2 | 0.76% | 0.10% | 2 | 0.02% | 0.01% | 2 | 46.35 | 0.92 | 2 |
| 0 | 0.10% | 0.01% | 2 | 0.90% | 0.06% | 2 | 0.02% | 0.01% | 2 | 50.6 | 2.55 | 2 |

G029123

| | | | | | | | | | | | | |
|---|---|---|---|---|---|---|---|---|---|---|---|---|
| 60 | 97.68% | 0.04% | 2 | 0.47% | 0.66% | 2 | 0.46% | 0.01% | 2 | 95.45 | 0.07 | 2 |
| 30 | 53.86% | 0.40% | 2 | 0.19% | 0.27% | 2 | 0.00% | 0.00% | 2 | 72.5 | 1.70 | 2 |
| 15 | 31.51% | 2.00% | 2 | 0.23% | 0.33% | 2 | 0.00% | 0.00% | 2 | 62.8 | 3.25 | 2 |
| 7.5 | 16.83% | 2.14% | 2 | 0.00% | 0.00% | 2 | 0.00% | 0.00% | 2 | 56.25 | 0.35 | 2 |
| 3.75 | 7.72% | 2.36% | 2 | 0.56% | 0.21% | 2 | 0.18% | 0.25% | 2 | 52.35 | 0.07 | 2 |
| 1.88 | 3.37% | 0.94% | 2 | 0.34% | 0.48% | 2 | 0.00% | 0.00% | 2 | 48.4 | 0.14 | 2 |
| 0.94 | 0.39% | 0.05% | 2 | 0.18% | 0.25% | 2 | 0.00% | 0.00% | 2 | 48.9 | 0.85 | 2 |
| 0 | 0.00% | 0.00% | 2 | 0.00% | 0.00% | 2 | 0.00% | 0.00% | 2 | 47.3 | 3.25 | 2 |

G029128

| | | | | | | | | | | | | |
|---|---|---|---|---|---|---|---|---|---|---|---|---|
| 60 | 94.41% | 0.63% | 2 | 0.50% | 0.08% | 2 | 2.61% | 0.21% | 2 | 86.6 | 1.98 | 2 |
| 30 | 61.36% | 0.04% | 2 | 0.47% | 0.07% | 2 | 1.57% | 0.13% | 2 | 67.6 | 1.98 | 2 |
| 15 | 41.10% | 1.11% | 2 | 0.26% | 0.01% | 2 | 1.11% | 0.11% | 2 | 64.3 | 3.39 | 2 |
| 7.5 | 24.54% | 0.03% | 2 | 0.23% | 0.06% | 2 | 1.05% | 0.08% | 2 | 56.4 | 1.70 | 2 |
| 3.75 | 11.39% | 0.16% | 2 | 0.17% | 0.04% | 2 | 0.72% | 0.03% | 2 | 52.85 | 0.49 | 2 |
| 1.88 | 4.88% | 0.50% | 2 | 0.17% | 0.02% | 2 | 0.67% | 0.08% | 2 | 50.1 | 0.99 | 2 |
| 0.94 | 1.90% | 0.02% | 2 | 0.15% | 0.00% | 2 | 0.63% | 0.06% | 2 | 49.85 | 0.21 | 2 |
| 0 | 0.20% | 0.01% | 2 | 0.16% | 0.07% | 2 | 0.60% | 0.06% | 2 | 46.45 | 1.20 | 2 |

G029131

| | | | | | | | | | | | | |
|---|---|---|---|---|---|---|---|---|---|---|---|---|
| 60 | 94.92% | 0.02% | 2 | 1.48% | 0.09% | 2 | 1.86% | 0.22% | 2 | 95.3 | 0.99 | 2 |
| 30 | 79.21% | 2.08% | 2 | 1.49% | 0.07% | 2 | 1.24% | 0.02% | 2 | 86.75 | 4.74 | 2 |
| 15 | 64.70% | 2.53% | 2 | 1.03% | 0.15% | 2 | 0.65% | 0.32% | 2 | 79.85 | 0.21 | 2 |
| 7.5 | 41.12% | 2.09% | 2 | 1.32% | 0.35% | 2 | 0.38% | 0.02% | 2 | 70.3 | 0.57 | 2 |
| 3.75 | 23.58% | 0.18% | 2 | 1.20% | 0.06% | 2 | 0.27% | 0.05% | 2 | 60.1 | 1.70 | 2 |
| 1.88 | 11.88% | 0.04% | 2 | 1.20% | 0.14% | 2 | 0.23% | 0.09% | 2 | 55.15 | 1.06 | 2 |
| 0.94 | 4.51% | 0.14% | 2 | 1.05% | 0.11% | 2 | 0.15% | 0.01% | 2 | 50.2 | 1.56 | 2 |
| 0 | 0.26% | 0.04% | 2 | 1.07% | 0.15% | 2 | 0.10% | 0.02% | 2 | 44.95 | 0.49 | 2 |

G029171

| | | | | | | | | | | | | |
|---|---|---|---|---|---|---|---|---|---|---|---|---|
| 60 | 81.24% | 3.07% | 2 | 1.68% | 0.07% | 2 | 0.80% | 0.15% | 2 | 94.4 | 1.98 | 2 |
| 30 | 26.12% | 2.63% | 2 | 1.70% | 0.16% | 2 | 0.21% | 0.08% | 2 | 63.25 | 3.46 | 2 |
| 15 | 15.27% | 1.25% | 2 | 1.81% | 0.12% | 2 | 0.29% | 0.07% | 2 | 56.95 | 0.35 | 2 |
| 7.5 | 8.44% | 0.58% | 2 | 1.43% | 0.01% | 2 | 0.21% | 0.08% | 2 | 53.6 | 0.28 | 2 |
| 3.75 | 4.16% | 0.26% | 2 | 1.78% | 0.11% | 2 | 0.17% | 0.05% | 2 | 53.9 | 2.12 | 2 |
| 1.88 | 2.45% | 0.10% | 2 | 1.67% | 0.03% | 2 | 0.20% | 0.00% | 2 | 51 | 4.10 | 2 |
| 0.94 | 1.49% | 0.23% | 2 | 2.86% | 2.01% | 2 | 0.78% | 0.82% | 2 | 50.75 | 2.76 | 2 |
| 0 | 0.16% | 0.02% | 2 | 1.51% | 0.14% | 2 | 0.18% | 0.01% | 2 | 47.15 | 1.06 | 2 |

G029172

| | | | | | | | | | | | | |
|---|---|---|---|---|---|---|---|---|---|---|---|---|
| 60 | 92.73% | 0.76% | 2 | 1.29% | 0.12% | 2 | 2.73% | 0.33% | 2 | 97.2 | 0.28 | 2 |
| 30 | 50.99% | 1.65% | 2 | 0.94% | 0.22% | 2 | 6.70% | 0.11% | 2 | 75.45 | 2.19 | 2 |
| 15 | 28.49% | 1.14% | 2 | 0.96% | 0.02% | 2 | 8.41% | 0.22% | 2 | 64.8 | 2.83 | 2 |
| 7.5 | 15.76% | 0.07% | 2 | 0.69% | 0.07% | 2 | 7.58% | 0.46% | 2 | 59.55 | 0.35 | 2 |
| 3.75 | 7.75% | 0.38% | 2 | 0.77% | 0.21% | 2 | 7.21% | 0.59% | 2 | 55.95 | 0.78 | 2 |
| 1.88 | 3.11% | 0.18% | 2 | 0.75% | 0.04% | 2 | 8.28% | 0.66% | 2 | 50.45 | 5.44 | 2 |
| 0.94 | 0.88% | 0.05% | 2 | 1.26% | 0.75% | 2 | 24.29% | 23.82% | 2 | 49.45 | 3.61 | 2 |
| 0 | 0.08% | 0.02% | 2 | 0.60% | 0.19% | 2 | 7.52% | 0.26% | 2 | 46.2 | 0.99 | 2 |

G026584

| | | | | | | | | | | | | |
|---|---|---|---|---|---|---|---|---|---|---|---|---|
| 60 | 95.17% | 0.99% | 2 | 1.26% | 0.25% | 2 | 2.17% | 0.03% | 2 | 99.05 | 0.07 | 2 |
| 30 | 84.51% | 1.04% | 2 | 1.59% | 0.03% | 2 | 1.86% | 0.12% | 2 | 93.45 | 0.07 | 2 |
| 15 | 70.25% | 0.04% | 2 | 1.26% | 0.05% | 2 | 1.92% | 0.01% | 2 | 86.35 | 2.47 | 2 |
| 7.5 | 49.19% | 2.18% | 2 | 1.19% | 0.32% | 2 | 1.29% | 0.07% | 2 | 76.45 | 2.05 | 2 |
| 3.75 | 27.90% | 2.71% | 2 | 0.84% | 0.15% | 2 | 0.92% | 0.24% | 2 | 66.15 | 0.92 | 2 |
| 1.88 | 10.85% | 0.07% | 2 | 1.01% | 0.14% | 2 | 0.93% | 0.19% | 2 | 52.75 | 7.00 | 2 |
| 0.94 | 3.17% | 0.16% | 2 | 1.11% | 0.06% | 2 | 1.21% | 0.03% | 2 | 49.4 | 1.56 | 2 |
| 0 | 0.08% | 0.03% | 2 | 1.01% | 0.26% | 2 | 1.35% | 0.53% | 2 | 43 | 2.83 | 2 |

G026585

| | | | | | | | | | | | | |
|---|---|---|---|---|---|---|---|---|---|---|---|---|
| 60 | 86.56% | 0.77% | 2 | 1.17% | 0.01% | 2 | 9.62% | 0.42% | 2 | 96.35 | 1.06 | 2 |
| 30 | 49.55% | 0.28% | 2 | 1.23% | 0.02% | 2 | 5.08% | 0.37% | 2 | 74.3 | 3.25 | 2 |
| 15 | 29.91% | 0.35% | 2 | 1.13% | 0.27% | 2 | 3.10% | 0.05% | 2 | 65.15 | 3.61 | 2 |
| 7.5 | 16.67% | 0.14% | 2 | 1.08% | 0.03% | 2 | 1.72% | 0.11% | 2 | 57.2 | 1.13 | 2 |
| 3.75 | 8.49% | 0.35% | 2 | 1.01% | 0.09% | 2 | 1.10% | 0.05% | 2 | 51.85 | 6.43 | 2 |
| 1.88 | 3.16% | 0.60% | 2 | 1.07% | 0.09% | 2 | 0.64% | 0.19% | 2 | 51.15 | 4.31 | 2 |
| 0.94 | 1.04% | 0.15% | 2 | 0.95% | 0.02% | 2 | 0.52% | 0.11% | 2 | 45.95 | 2.19 | 2 |
| 0 | 0.05% | 0.05% | 2 | 0.95% | 0.11% | 2 | 0.40% | 0.05% | 2 | 47.9 | 2.69 | 2 |

### Example 10: Screening of AAVS1 Guides with Nme2Cas9

Guide RNAs targeting AAVS1 were screened for editing efficacy in T cells by assessing editing by NGS, following AAVS1 editing by mRNA delivery.

T Cell Preparation and Editing with RNA Electroporation

T cells single donor (no. 613) were prepared as described in Example 1 using 2.5% human AB serum (GeminiBio, Cat. 100-512) in the T Cell growth media. T cells were electroporated with sgRNA targeting the AAVS1 gene using mRNA electroporation as described in Example 1 with the following exceptions.

This study used mRNA encoding Nme2 Cas9 (SEQ ID No: 825) instead of mRNAs encoding Nme2 BC22n base editor and UGI, respectively. Nme2 Cas9 electroporation mix was prepared with 1×10⁵ T cells, 30 ng/μL of Nme2Cas9 mRNA and 40 pmols of sgRNA in a final volume of 20 μL of P3 electroporation buffer. On day 7 post-electroporation, DNA samples were subjected to PCR and subsequent NGS analysis as described in Example 1.

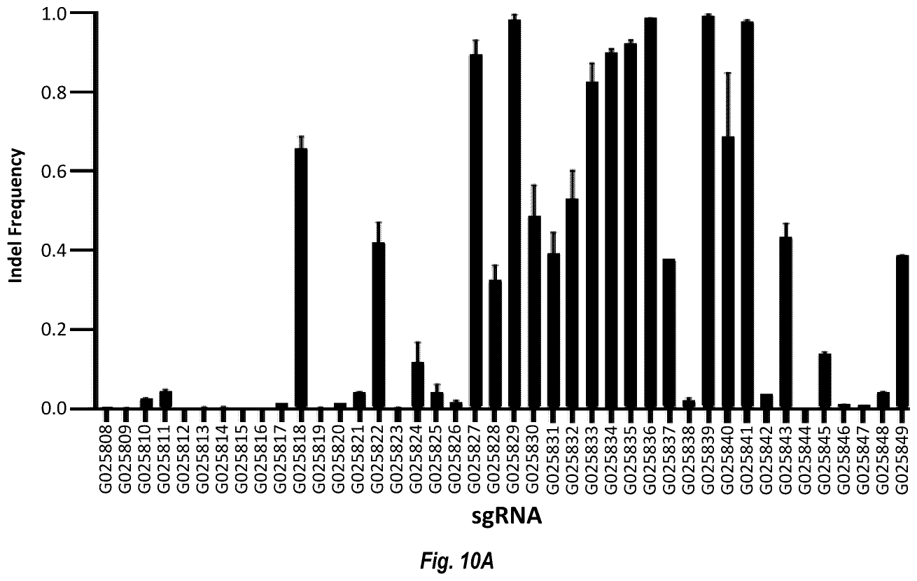

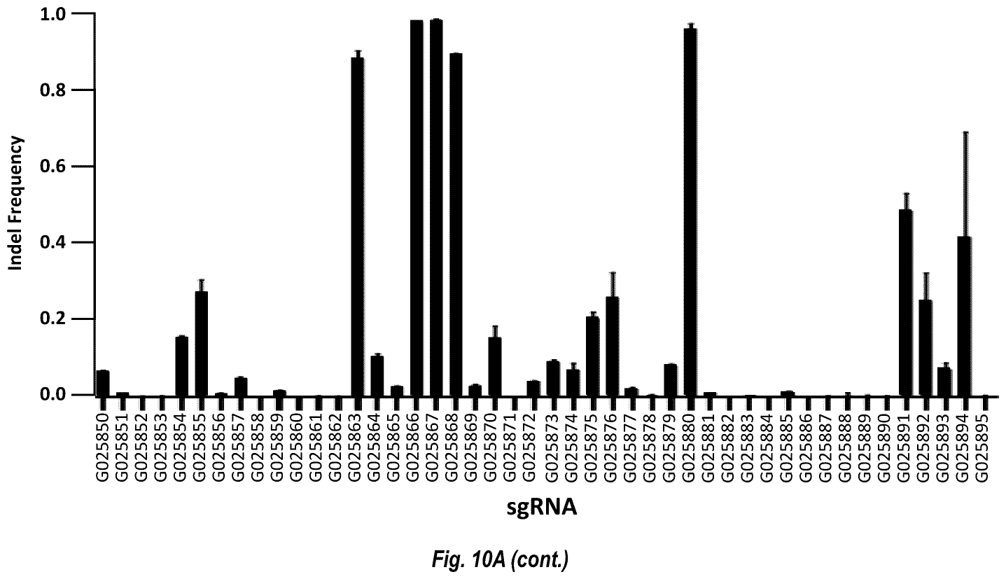

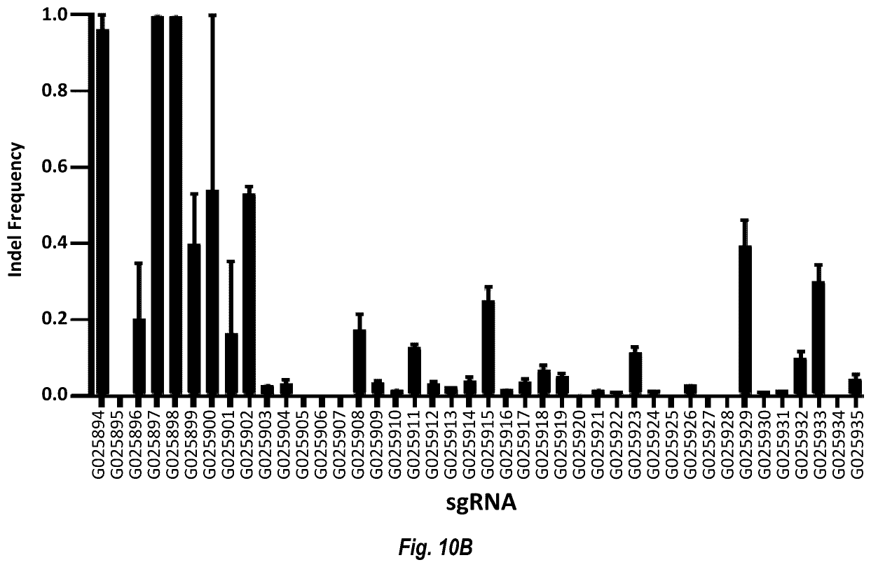

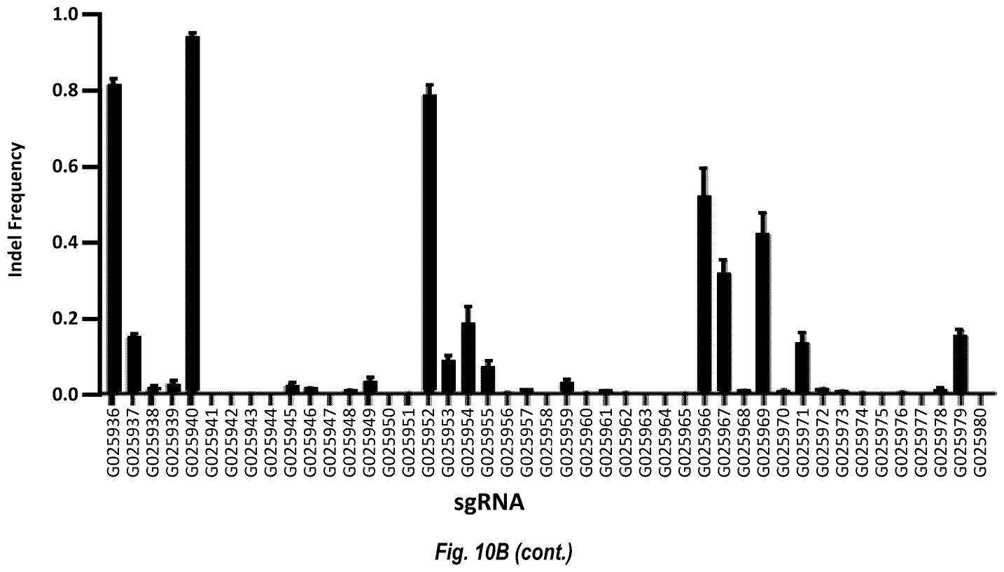

Table 17 shows mean percent indels at the AAVS1 gene with Nme2Cas9 guides. FIGS. 10A-10B show editing at the AAVS1 gene represented as indel frequency.

TABLE 17
Mean percent indels at the AAVS1 locus
as a percentage of total NGS reads

| | | | Guide | | | |
|---|---|---|---|---|---|---|
| Guide ID | Mean | SD | ID | Mean | SD | N |
| G025808 | 0.6 | 0 | G025894 | 95.9 | 4.1 | 2 |
| G025809 | 0.25 | 0.05 | G025895 | 0.15 | 0.05 | 2 |
| G025810 | 2.3 | 0.3 | G025896 | 20.1 | 10.3 | 2 |
| G025811 | 4.25 | 0.45 | G025897 | 99.75 | 0.05 | 2 |
| G025812 | 0.2 | 0 | G025898 | 99.55 | 0.05 | 2 |
| G025813 | 0.3 | 0.1 | G025899 | 39.5 | 9.4 | 2 |
| G025814 | 0.45 | 0.05 | G025900 | 54.1 | 41.9 | 2 |
| G025815 | 0.3 | 0 | G025901 | 16.3 | 13.4 | 2 |
| G025816 | 0 | 0 | G02502 | 52.75 | 1.55 | 2 |
| G025817 | 0.9 | 0.1 | G025903 | 2.7 | 0.2 | 2 |
| G025818 | 65.55 | 2.25 | G025904 | 2.7 | 1.1 | 2 |
| G025819 | 0.35 | 0.05 | G025905 | 0.5 | 0 | 2 |
| G025820 | 1.1 | 0 | G025906 | 0.4 | 0 | 2 |
| G025821 | 3.8 | 0.3 | G025907 | 0.1 | 0 | 2 |
| G025822 | 41.7 | 3.8 | G025908 | 16.9 | 3.1 | 2 |
| G025823 | 0.35 | 0.05 | G025909 | 2.95 | 0.35 | 2 |
| G025824 | 11.25 | 3.95 | G025910 | 1.45 | 0.15 | 2 |
| G025825 | 3.95 | 1.55 | G025911 | 12.3 | 0.5 | 2 |
| G025826 | 1.25 | 0.65 | G025912 | 2.75 | 0.35 | 2 |
| G025827 | 89.3 | 2.6 | G025913 | 1.85 | 0.15 | 2 |
| G025828 | 32.05 | 2.95 | G025914 | 3.7 | 0.4 | 2 |
| G025829 | 98.1 | 1 | G025915 | 24.75 | 2.25 | 2 |
| G025830 | 48.45 | 5.65 | G025916 | 1.4 | 0.2 | 2 |
| G025831 | 39 | 3.9 | G025917 | 3.25 | 0.65 | 2 |
| G025832 | 52.9 | 5.1 | G025918 | 6.15 | 0.85 | 2 |
| G025833 | 82.15 | 3.55 | G025919 | 4.7 | 0.4 | 2 |
| G025834 | 89.75 | 0.75 | G025920 | 0.25 | 0.05 | 2 |
| G025835 | 92 | 0.8 | G025921 | 1.15 | 0.35 | 2 |
| G025836 | 98.55 | 0.05 | G025922 | 0.75 | 0.25 | 2 |
| G025837 | 37.35 | 0.15 | G025923 | 10.9 | 1.1 | 2 |
| G025838 | 1.7 | 0.7 | G025924 | 1.05 | 0.05 | 2 |
| G025839 | 99.2 | 0.3 | G025925 | 0.2 | 0 | 2 |
| G025840 | 68.55 | 11.45 | G025926 | 2.5 | 0.2 | 2 |
| G025841 | 97.45 | 0.45 | G025927 | 0.2 | 0 | 2 |
| G025842 | 3.45 | 0.05 | G025928 | 0.2 | 0 | 2 |
| G025843 | 43 | 2.7 | G025929 | 39.1 | 5 | 2 |
| G025844 | 0.1 | 0 | G025930 | 0.8 | 0.1 | 2 |
| G025845 | 13.5 | 0.6 | G025931 | 1.1 | 0.2 | 2 |
| G025846 | 0.9 | 0.1 | G025932 | 9.7 | 1.2 | 2 |
| G025847 | 0.55 | 0.15 | G025933 | 29.6 | 3.3 | 2 |
| G025848 | 3.9 | 0.3 | G025934 | 0.5 | 0 | 2 |
| G025849 | 38.4 | 0.3 | G025935 | 4.15 | 0.75 | 2 |
| G025850 | 6.7 | 0.2 | G025936 | 85.1 | 1.2 | 2 |
| G025851 | 1 | 0 | G025937 | 15.5 | 0.6 | 2 |
| G025852 | 0.15 | 0.05 | G025938 | 1.25 | 0.35 | 2 |
| G025853 | 0.25 | 0.05 | G025939 | 2.75 | 0.55 | 2 |
| G025854 | 15.85 | 0.05 | G025940 | 98.3 | 0.6 | 2 |
| G025855 | 27.4 | 2.3 | G025941 | 0.1 | 0 | 2 |
| G025856 | 0.95 | 0.05 | G025942 | 0.25 | 0.05 | 2 |
| G025857 | 4.9 | 0.2 | G025943 | 0.25 | 0.05 | 2 |
| G025858 | 0.3 | 0 | G025944 | 0.15 | 0.05 | 2 |
| G025859 | 1.55 | 0.15 | G025945 | 2.2 | 0.3 | 2 |
| G025860 | 0.2 | 0 | G025946 | 1.6 | 0.2 | 2 |
| G025861 | 0.25 | 0.05 | G025947 | 0.2 | 0 | 2 |
| G025862 | 0.15 | 0.05 | G025948 | 1.1 | 0.1 | 2 |
| G025863 | 88.6 | 1.4 | G025949 | 3.45 | 0.55 | 2 |
| G025864 | 10.55 | 0.55 | G025950 | 0.2 | 0 | 2 |
| G025865 | 2.7 | 0.1 | G025951 | 0.35 | 0.05 | 2 |
| G025866 | 98.35 | 0.05 | G025952 | 82.2 | 1.9 | 2 |
| G025867 | 98.75 | 0.05 | G025953 | 9.25 | 0.65 | 2 |
| G025868 | 89.85 | 0.05 | G025954 | 19.2 | 3.3 | 2 |
| G025869 | 3.1 | 0.1 | G025955 | 7.5 | 1.1 | 2 |
| G025870 | 15.45 | 2.15 | G025956 | 0.55 | 0.05 | 2 |
| G025871 | 0.3 | 0 | G025957 | 1 | 0.1 | 2 |
| G025872 | 4.15 | 0.05 | G025958 | 0.5 | 0 | 2 |
| G025873 | 9.1 | 0.4 | G025959 | 3.05 | 0.45 | 2 |
| G025874 | 7.15 | 1.15 | G025960 | 0.4 | 0.1 | 2 |
| G025875 | 20.6 | 1.1 | G025961 | 0.95 | 0.05 | 2 |
| G025876 | 25.95 | 4.65 | G025962 | 0.4 | 0.1 | 2 |
| G025877 | 2.2 | 0.2 | G025963 | 0.1 | 0 | 2 |
| G025878 | 0.55 | 0.05 | G025964 | 0.15 | 0.05 | 2 |
| G025879 | 8.25 | 0.25 | G025965 | 0.35 | 0.05 | 2 |
| G025880 | 96.45 | 0.85 | G025966 | 54.5 | 5.4 | 2 |
| G025881 | 1.2 | 0 | G025967 | 32.95 | 2.55 | 2 |
| G025882 | 0.2 | 0 | G025968 | 1.05 | 0.05 | 2 |
| G025883 | 0.6 | 0 | G025969 | 43.9 | 4 | 2 |
| G025884 | 0.3 | 0 | G025970 | 0.9 | 0.3 | 2 |
| G025885 | 1.35 | 0.05 | G025971 | 14 | 2 | 2 |
| G025886 | 0.1 | 0 | G025972 | 1.55 | 0.05 | 2 |
| G025887 | 0.25 | 0.05 | G025973 | 0.95 | 0.05 | 2 |
| G025888 | 0.45 | 0.45 | G025974 | 0.45 | 0.05 | 2 |
| G025889 | 0.3 | 0.1 | G025975 | 0.25 | 0.05 | 2 |
| G025890 | 0.25 | 0.05 | G025976 | 0.65 | 0.05 | 2 |
| G025891 | 48.8 | 3.1 | G025977 | 0.4 | 0 | 2 |
| G025892 | 25.25 | 5.05 | G025978 | 1 | 0.3 | 2 |
| G025893 | 7.3 | 1.1 | G025979 | 15.95 | 1.05 | 2 |
| G025894 | 41.85 | 19.35 | G025980 | 0.1 | 0 | 2 |
| G025895 | 0.35 | 0.05 | | | | |

### Example 11: Dose Response of AAVS1 Guides with Nme2Cas9

A dilution series of AAVS1 guide RNAs were screened for editing efficacy by dose response in T cells by assessing editing by NGS following AAV S1 editing by mRNA delivery. A four-point dose response curve was generated for each sgRNA by titrating each sgRNA with a fixed concentration of mRNA encoding Nme2 Cas9 (SEQ ID NO: 825) in T cells using mRNA electroporation.

T cells single donor (no. 613) were prepared and activated as described in Example 1 using 2.5% o human AB serum (GeminiBio, Cat. 100-512) in T Cell growth media (TCGM). T cells were electroporated with serially diluted sgRNA targeting the AAVS1 gene using mRNA electroporation as described in Example 1 with the following exceptions. This study used mRNA encoding Nme2 Cas9 instead of mRNAs encoding Nme2 BC22n base editor and UGI, respectively. Cas9 electroporation mix was prepared with 1×10⁵ T cells, 30 ng/L of Nme2Cas9 mRNA and 40, 13.3, 4.4 and 1.5 pmols of sgRNA in a final volume of 20 μL of P3 electroporation buffer. On day 3 post-editing, T cells were harvested and subjected to PCR and NGS sequencing as described in Example 1.

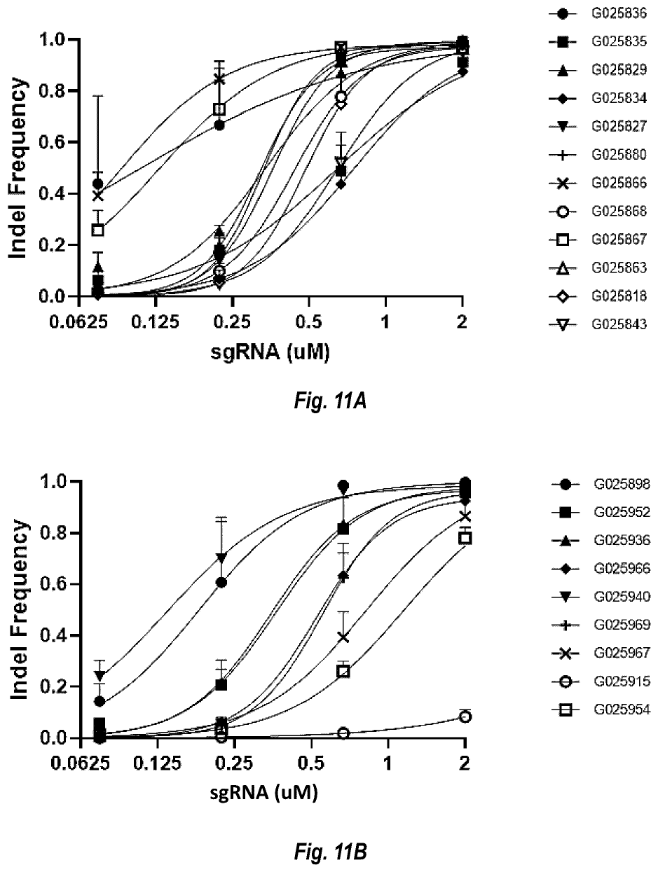

Table 18 and FIGS. 11A-11B show the mean indel frequency at various AASV1 sgRNA concentrations.

TABLE 18
Mean indel frequency at AAVS1

| sgRNA | G025836 | | | G025835 | | | G025829 | | | G025834 | | |
|---|---|---|---|---|---|---|---|---|---|---|---|---|
| (uM) | Mean | SD | N | Mean | SD | N | Mean | SD | N | Mean | SD | N |
| 2.00 | 1.00 | 0.00 | 2 | 0.91 | 0.01 | 2 | 1.00 | 0.00 | 2 | 0.88 | 0.03 | 2 |
| 0.67 | 0.93 | 0.02 | 2 | 0.49 | 0.07 | 2 | 0.87 | 0.03 | 2 | 0.44 | 0.03 | 2 |
| 0.22 | 0.67 | 0.16 | 2 | 0.18 | 0.00 | 2 | 0.25 | 0.02 | 2 | 0.07 | 0.02 | 2 |
| 0.07 | 0.44 | 0.24 | 2 | 0.06 | 0.03 | 2 | 0.12 | 0.04 | 2 | 0.02 | 0.01 | 2 |

| | G025827 | | | G025880 | | | G025866 | | | G025868 | | |
|---|---|---|---|---|---|---|---|---|---|---|---|---|
| sgRNA | Mean % | | | Mean % | | | Mean % | | | Mean % | | |
| (uM) | editing | SD | N | editing | SD | N | editing | SD | N | editing | SD | N |
| 2.00 | 0.99 | 0.00 | 2 | 0.97 | 0.00 | 2 | 0.97 | 0.00 | 2 | 0.97 | 0.00 | 2 |
| 0.67 | 0.90 | 0.03 | 2 | 0.93 | 0.04 | 2 | 0.97 | 0.00 | 2 | 0.78 | 0.09 | 2 |
| 0.22 | 0.14 | 0.00 | 2 | 0.16 | 0.05 | 2 | 0.85 | 0.05 | 2 | 0.10 | 0.02 | 2 |
| 0.07 | 0.03 | 0.00 | 2 | 0.03 | 0.01 | 2 | 0.39 | 0.06 | 2 | 0.02 | 0.01 | 2 |
| | G025867 | | | G025863 | | | G025818 | | | G025843 | | |
| sgRNA | Mean % | | | Mean % | | | Mean % | | | Mean % | | |
| (uM) | editing | SD | N | editing | SD | N | editing | SD | N | editing | SD | N |
| 2.00 | 0.97 | 0.01 | 2 | 0.97 | 0.00 | 2 | 0.97 | 0.00 | 2 | 0.95 | 0.00 | 2 |
| 0.67 | 0.97 | 0.01 | 2 | 0.92 | 0.02 | 2 | 0.75 | 0.08 | 2 | 0.52 | 0.08 | 2 |
| 0.22 | 0.73 | 0.08 | 2 | 0.19 | 0.04 | 2 | 0.05 | 0.01 | 2 | 0.05 | 0.02 | 2 |
| 0.07 | 0.26 | 0.05 | 2 | 0.04 | 0.01 | 2 | 0.01 | 0.00 | 2 | 0.01 | 0.00 | 2 |

### Example 12. Screening of Insertion Guide RNAs with Nme2Cas9 or SpyCas9

A select sgRNA targeting CIITA (G023477) was redesigned for AAV insertion and evaluated for insertion efficacy in T cells. Cells were edited using Nme2Cas9 and an AAV encoding an insertion template including a GFP reporter gene flanked by homology arms to the guide cut site. Insertion efficiency was assessed by measuring luminescence and the absence of HLA-DP, DQ, DR expression by flow cytometry and editing at AAVS1 was assessed by NGS.

12.1 mRNA Electroporation and AAV Transduction of T Cells

T cells from a single donor (no. 613) were prepared and activated as described in Example 1. T cells were electroporated with sgRNA targeting the CIITA gene using mRNA electroporation as described in Example 1 except for the following differences. This study used mRNA encoding Nme2 Cas9 (SEQ ID No: 825) instead of mRNAs encoding Nme2 BC22n base editor and UGI, respectively. Guide G028533 targeting targeting CIITA was editing using mRNA encoding SpCas9 (SEQ ID No: 816). Nme2 Cas9 electroporation mix was prepared with 1×10⁵ T cells in P3 buffer (Lonza), 600 ng of mRNA encoding Cas9 and 20 pmoles of sgRNA. Fifteen minutes after electroporation, T cells were transduced with AAV. Briefly, AAV encoding green fluorescent protein flanked by homology arms bracketing the guide cut site was added to 80 ul of CTS Optimizer T cell growth media supplemented with in new flat-bottom 96-well plates with final multiplicity of infection (MOI) of 300,000. Electroporated T cells were added to the resulting plates and incubated at 37° C. Twenty-four hours post-electroporation and transduction, cells were split 1:2 in 2 U-bottom plates replenished with CTS Optimizer media supplemented with cytokines.

### 12.2 Flow Cytometry and NGS Sequencing

On day 7 post-electroporation, edited T cell samples were subjected to PCR and NGS analysis as described in Example 1.

Edited T cells were phenotyped by flow cytometry 10 days post electroporation to determine HLA II-DR, DP, DQ protein expression. Briefly, T cells were incubated for 30 min at 4° C. with a mixture of antibodies against CD3 (BioLegend, Cat. No. 317338), CD4 (BioLegend, Cat. No. 317434), CD8 (BioLegend, Cat. No. 301046), Viakrome (Beckman Coulter, Cat. No. C36628) diluted at 1:100 and HLA II-DR, DP, DQ (BioLegend, Cat. No. 361711) diluted at 1:50 in cell staining buffer (BioLegend, Cat. No. 420201). Cells were subsequently washed and resuspended in 100 μL of cell staining buffer. Cells were then processed on a Cytoflex flow cytometer (Beckman Coulter) and analyzed using the FlowJo™ software package. T cells were gated based on size, shape, viability, as well as CD8, HLA II-DR, DP, DQ, and GFP expression. Table 19 shows the percentage of T cells with HLA II-DR, DP, DQ negative cells, HLA II-DR, DP, DQ negative cells with GFP, and mean percent indel frequency.

TABLE 19
Mean percentages of HLA II-DP, DR, DQ negative cells and HLA-
DP, DQ, DR negative, GFP positive cells.; Mean percent indel
frequency of the CIITA loci as a percentage of total NGS reads

| | | | | % MHC Class II | | | % MHC Class II | | |
|---|---|---|---|---|---|---|---|---|---|
| | % Indel Frequency | | | negative | | | negative, GFP+ | | |
| Guide | Mean | SD | n | Mean | SD | n | Mean | SD | n |
| Untreated | 0 | n/a | 1 | 46.5 | n/a | 1 | 0 | 0 | 2 |
| AAV only | 0 | n/a | 1 | 47.20 | 0.24 | 2 | 5.05 | 0.50 | 3 |
| G026584 | 0.86 | 0.02 | 3 | 87.10 | 0.82 | 2 | 59.47 | 2.30 | 3 |
| G026588 | 0.98 | 0.002 | 3 | 75.40 | 0.82 | 2 | 14.57 | 14.57 | 3 |

### Example 13. Dose Dependence of Additional TRAC Guide RNAs with Nme2Cas9

Guide RNAs targeting TRAC were screened for editing efficacy in T cells. TRAC editing was assessed by NGS for each concentration of sgRNA. T cells from a single donor were prepared and activated as described in Example 1 using 2.5% human AB serum (GeminiBio, Cat. 100-512) in T Cell growth media (TCGM). T cells were electroporated as described in Example 1 with sgRNAs targeting the TRAC locus at the concentration listed in Table 20 with the following exceptions. This study used 30 ng/μL mRNA encoding Nme2 Cas9 instead of mRNAs encoding Nme2 BC22n base editor and UGI.

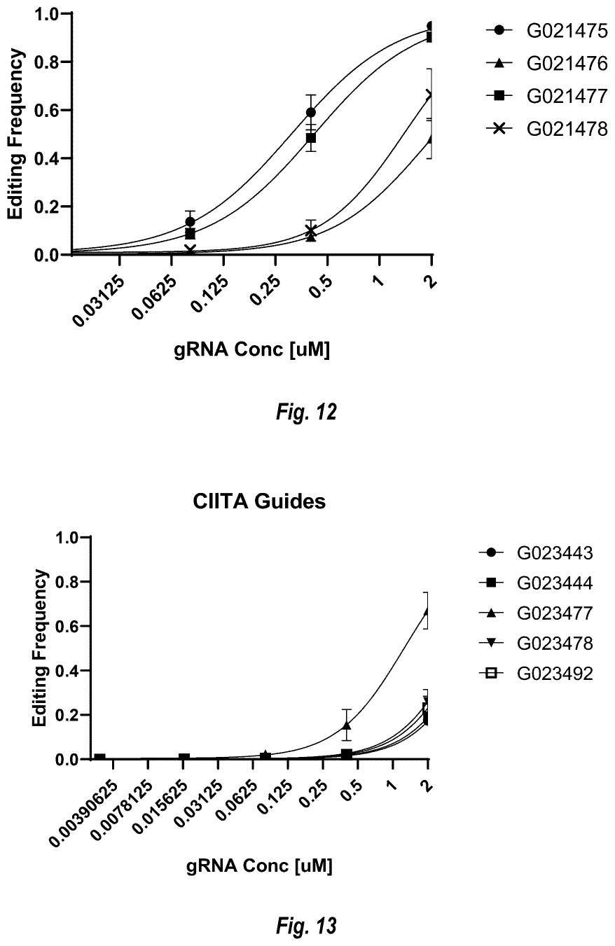

On day 3 post-electroporation, T cells were harvested and subjected to PCR and subsequent NGS analysis as described in Example 1. Table 20 and FIG. 12 show the mean editing frequency at various TRAC sgRNA concentrations.

TABLE 20
Mean editing frequency at the TRAC locus

| sgRNA | G021475 | | | G021476 | | | G021477 | | | G021478 | | |
|---|---|---|---|---|---|---|---|---|---|---|---|---|
| (uM) | Mean | SD | N | Mean | SD | N | Mean | SD | N | Mean | SD | N |
| 2.00 | 0.95 | 0.01 | 2 | 0.48 | 0.06 | 2 | 0.90 | 0.01 | 2 | 0.66 | 0.08 | 2 |
| 0.40 | 0.59 | 0.05 | 2 | 0.08 | 0.01 | 2 | 0.48 | 0.04 | 2 | 0.10 | 0.03 | 2 |
| 0.08 | 0.13 | 0.03 | 2 | 0.01 | 0.00 | 2 | 0.09 | 0.02 | 2 | 0.02 | 0.01 | 2 |
| 0.02 | 0.02 | 0.00 | 2 | 0.00 | 0.00 | 2 | 0.01 | 0.01 | 2 | 0.01 | 0.00 | 2 |

### Example 14. Dose Dependence for Select CIITA Guide RNAs with Nme2Cas9

Guide RNAs targeting CIITA were screened for editing efficacy by dose response in T cells. CIITA editing was assessed by NGS for each concentration of sgRNA. A five-point dilution series was generated for each sgRNA by titrating each sgRNA with a fixed concentration of mRNA encoding Nme2 Cas9 in T cells using electroporation.

T cells were prepared from single donor as described in Example 1. T cells were electroporated with mRNA encoding Nme2 Cas9 and CIITA-targeting sgRNAs at the concentrations listed in Table 21 as described in Example 1. On day 3 post-editing, T cell samples were collected and subjected to PCR and NGS analysis as described in Example 1. Table 21 and FIG. 13 show the NGS editing results.

TABLE 21
Mean editing frequency at the CIITA locus.

| | sgRNA | | | |
|---|---|---|---|---|
| Guide | (uM) | Mean | SD | N |
| G023443 | 2 | 0.17 | 0.01 | 4 |
| | 0.4 | 0.01 | 0.00 | 4 |
| | 0.08 | 0.00 | 0.00 | 4 |
| | 0.016 | 0.00 | 0.00 | 4 |
| | 0.003 | 0.00 | 0.00 | 4 |
| G023444 | 2 | 0.19 | 0.01 | 4 |
| | 0.4 | 0.02 | 0.01 | 4 |
| | 0.08 | 0.00 | 0.00 | 4 |
| | 0.016 | 0.00 | 0.00 | 4 |
| | 0.003 | 0.00 | 0.00 | 4 |
| G023477 | 2 | 0.67 | 0.07 | 4 |
| | 0.4 | 0.16 | 0.06 | 4 |
| | 0.08 | 0.03 | 0.00 | 4 |
| | 0.016 | 0.00 | 0.00 | 4 |
| | 0.003 | 0.00 | 0.00 | 4 |
| G023478 | 2 | 0.26 | 0.05 | 4 |
| | 0.4 | 0.03 | 0.01 | 4 |
| | 0.08 | 0.01 | 0.00 | 4 |
| | 0.016 | 0.00 | 0.00 | 4 |
| | 0.003 | 0.00 | 0.00 | 4 |
| G023492 | 2 | 0.23 | 0.05 | 4 |
| | 0.4 | 0.02 | 0.01 | 4 |
| | 0.08 | 0.00 | 0.00 | 4 |
| | 0.016 | 0.00 | 0.00 | 4 |
| | 0.003 | 0.00 | 0.00 | 4 |

### Example 15. Dose Responsiveness Analysis in Non-Activated T Cells

Guide RNAs were screened for editing efficacy in non-activated T cells by assessing editing frequency by NGS or by flow cytometry following lipid nanoparticle (LNP) delivery.

### 15.1 T Cell Preparation

Isolated, cryopreserved T cells from 3 donors were thawed in a water bath on Day 0 and plated at a density of 1×10⁶ cells/mL in TCAM media containing CTS OpTmizer T Cell Expansion SFM and T Cell Expansion Supplement (ThermoFisher Cat. A1048501), 1× Penicillin-Streptomycin, 1× Glutamax, 10 mM HEPES, 200 U/mL recombinant human interleukin-2 (Peprotech, Cat. 200-02), 5 ng/mL recombinant human interleukin 7 (Peprotech, Cat. 200-07), 5 ng/mL recombinant human interleukin 15 (Peprotech, Cat. 200-15), 2.5% human AB serum (GeminiBio, Cat. 100-512).

### 15.2 T Cell Engineering

LNPs were generally prepared as described in Example 1. Lipid nanoparticles in this example were prepared with molar ratios of 35 Lipid A: 47.5 cholesterol: 15 DSPC: 2.5 PEG2k-DMG. LNPs were made with a lipid amine to RNA phosphate (N:P) molar ratio of about 6. LNPs were formulated with a single RNA species. LNPs were delivered to T cells in TCAM media containing ApoE3 (Peprotech, Cat. 350-02).

On Day 1 (about 24 hours after thaw), T cells were centrifuged, resuspended, and plated at 100,000 cells/well in 50 μL/well TCGM with 2.5% human AB serum. LNPs were applied at 1 μg/mL total RNA cargo of Nme2 base editor mRNA and 0.5 μg/mL total RNA cargo of UGI mRNA along with 10 μg/mL of ApoE3. An LNP formulated with CIITA G026584 and an LNP formulated with HLA-A G028918 were mixed and applied in an eight point 2-fold serial dilution series starting with a high dose of 1.7 μg/mL CIITA G026584 and of 0.7 μg/mL HLA-A G028918. No LNPs were applied to “untreated” samples.

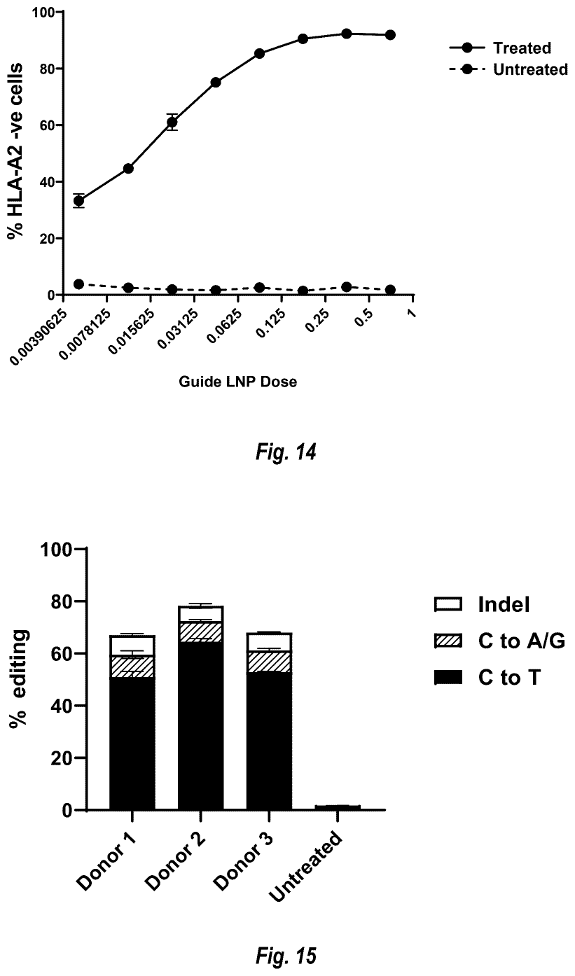

On Day 3 or Day 4, cells were washed and activated with 1:100 dilution of Trans Act (Miltenyi Biotec). Cells were incubated at 37° C. with regular cell splitting and the addition of fresh media. On Day 9, cells were harvested for analysis by flow cytometry and NGS analysis. For flow cytometric analysis, cells were washed in FACS buffer (PBS+2% FBS+2 mM EDTA). Cells were incubated in a cocktail of antibodies targeting CD4 (Biolegend 317434), CD8 (Biolegend 301046), CD3 (Biolegend 317336), HLA-A2 (Biolegend 343320), HLA-A3 (eBioscience, 11-5754-42), HLA-DR, DP, DQ (Biolegend 361712), and ViaKrome 808 Fixable Viability Dye (Beckman Coulter, C36628). T cells were subsequently washed and analyzed on a Cytoflex instrument (Beckman Coulter). Data analysis was performed using FlowJo software package (v.10.6.1). T cells were gated on size, viability, CD4 or CD8 expression, and expression of markers indicated in Table 22. Flow cytometry data for a representative donor that received about 48 hours of LNP exposure are shown in Table 22 and FIG. 14. Similar results were seen in all three donors and for both the 48- and 72-hour LNP exposure periods.

TABLE 22
Mean percent cells negative for HLA-A2 surface
expression for a representative donor.

| | | |
|---|---|---|
| G028918 | Treated | Untreated |

| (μg/mL) | Mean | SD | n | Mean | SD | n |
|---|---|---|---|---|---|---|
| 0.700 | 91.9 | 0.6 | 3 | 1.8 | 0.0 | 1 |
| 0.350 | 92.3 | 0.7 | 3 | 2.8 | 0.0 | 1 |
| 0.175 | 90.5 | 0.4 | 3 | 1.4 | 0.0 | 1 |
| 0.088 | 85.3 | 0.6 | 3 | 2.6 | 0.0 | 1 |
| 0.044 | 75.1 | 1.0 | 3 | 1.6 | 0.0 | 1 |
| 0.022 | 61.1 | 2.9 | 3 | 1.9 | 0.0 | 1 |
| 0.011 | 44.7 | 1.6 | 3 | 2.5 | 0.0 | 1 |
| 0.005 | 33.3 | 2.4 | 3 | 3.8 | 0.0 | 1 |

NGS analysis was performed on samples using 0.85 μg/mL G026584 and 0.35 μg/mL G028918 with 48-hour LNP exposure. NGS results are shown in Table 23 and FIG. 15.

TABLE 23
Mean percent editing at the CIITA locus.

| | C to T | | | C to A/G | | | Indel | | |
|---|---|---|---|---|---|---|---|---|---|
| Sample | Mean | SD | n | Mean | SD | n | Mean | SD | n |
| Donor 1 | 50.9 | 2.2 | 3 | 8.6 | 1.5 | 3 | 7.5 | 0.5 | 3 |
| Donor 2 | 64.5 | 1.2 | 3 | 7.9 | 0.6 | 3 | 5.8 | 0.9 | 3 |
| Donor 3 | 52.6 | 0.3 | 2 | 8.4 | 0.8 | 2 | 6.8 | 0.2 | 2 |
| Untreated | 0.3 | 0.0 | 2 | 1.3 | 0.1 | 2 | 0.2 | 0.0 | 2 |

### Example 16. Dose Response Analysis of Select Guide RNAs with LNP

Select HLA-A, TRAC, TRBC1 & TRBC2, and CIITA guide RNAs were screened for editing efficacy in T cells by assessing editing frequency by NGS and by flow cytometry following lipid nanoparticle (LNP) delivery.

### 16.1 T Cell Preparation & Activation

Isolated, cryopreserved T cells from 2 donors were thawed in a water bath on Day 0 and plated at a density of 1.5×10⁶ cells/mL in TCAM media containing CTS OpTmizer T Cell Expansion SFM and T Cell Expansion Supplement (Gibco Cat. A3705001), 1× Penicillin-Streptomycin, 1× Glutamax, 10 mM HEPES, 200 U/mL recombinant human interleukin-2 (Peprotech, Cat. 200-02), 5 ng/mL recombinant human interleukin 7 (Peprotech, Cat. 200-07), and 5 ng/mL recombinant human interleukin 15 (Peprotech, Cat. 200-15) and 2.5% human AB serum (GeminiBio, Cat. 100-512). On Day 1 (24 hours post thaw) cells were washed and activated with TransAct™ (1:100 dilution, Miltenyi Biotec).

### 16.2 LNP Formulation

LNPs were generally prepared as described in Example 1.6. Lipid nanoparticles in this example were prepared with molar ratios of 35 Lipid A: 47.5 cholesterol: 15 DSPC: 2.5 PEG2k-DMG. LNPs were made with a lipid amine to RNA phosphate (N:P) molar ratio of about 6. LNPs were formulated with a single RNA cargo of Nme2BC22 mRNA, UGI mRNA, or a sgRNA as listed in Table 24 and Table 25. LNPs were delivered to T cells in TCAM media containing ApoE3 (Peprotech, Cat. 350-02).

### 16.3 T Cell Engineering

On Day 3, T cells were centrifuged, resuspended, and plated at 100,000 cells/well in 100 μL/well of TCAM with 2.5% human AB serum to which 100 μL of corresponding LNP formulation were added. LNP formulation containing gRNA was added to T cells in at concentrations listed in Table 24 along with 1 μg/mL total RNA cargo of Nme2 base editor mRNA and 0.5 μg/mL total RNA cargo of UGI mRNA. No LNPs were applied to “untreated” samples.

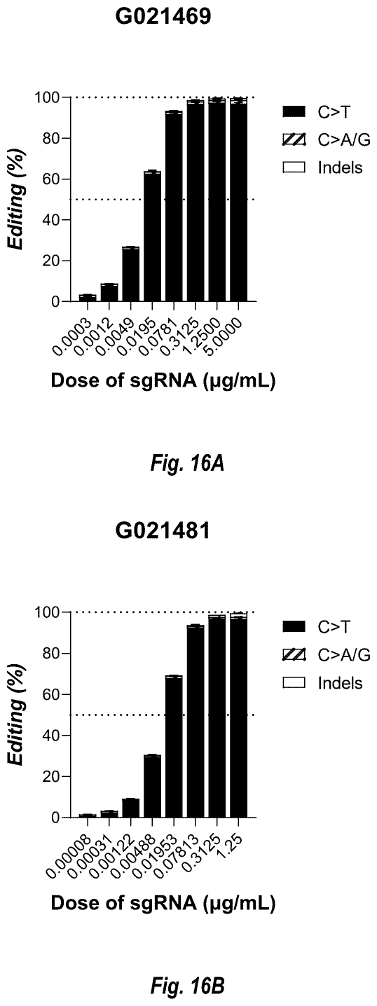

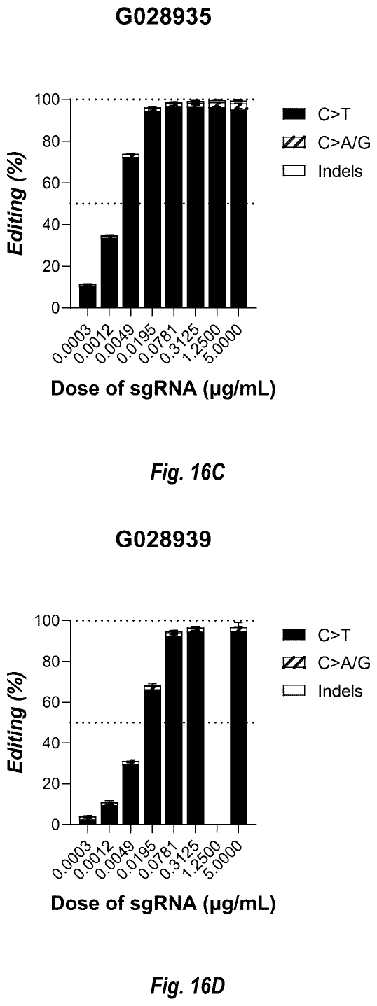

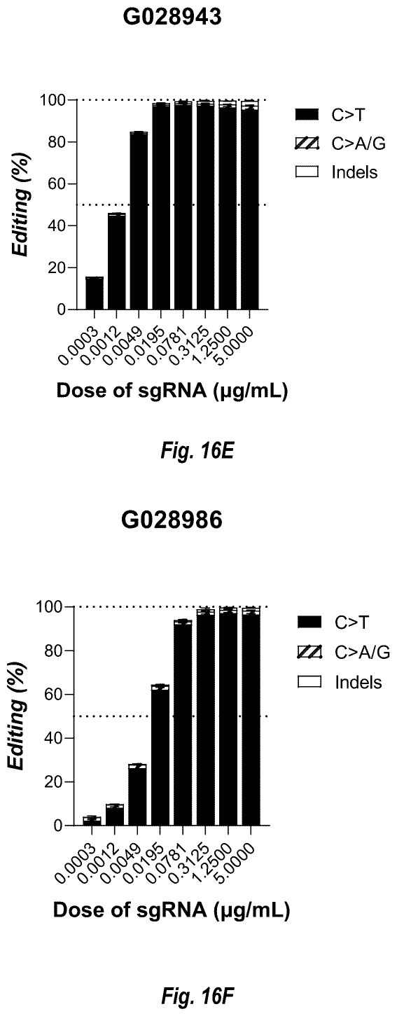

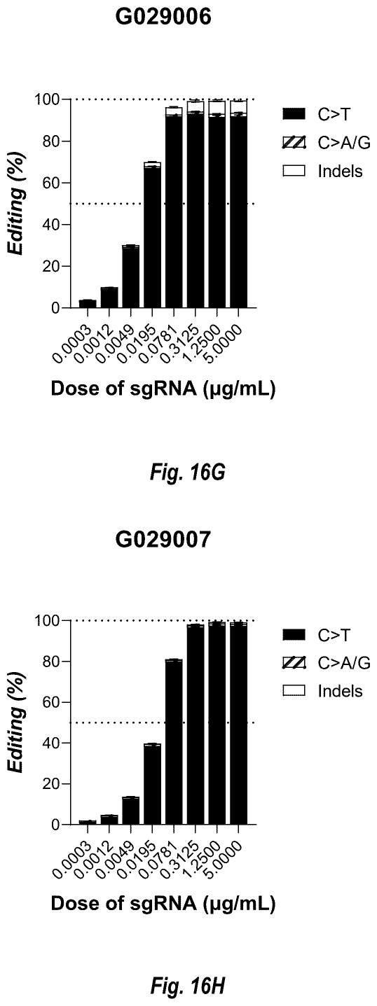

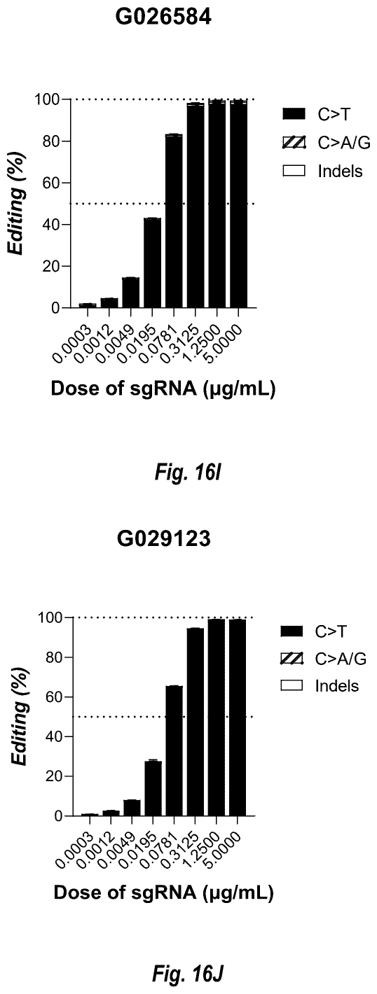

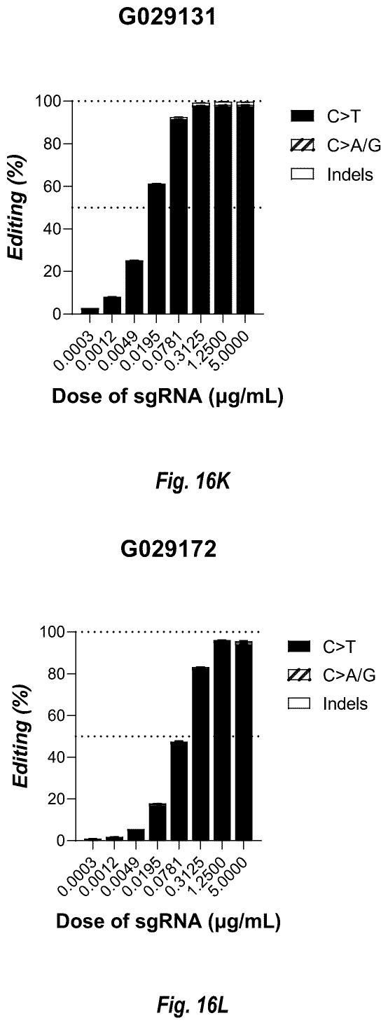

Beginning on Day 4, cells were split, and media refreshed regularly. On Day 7, a portion of cells were harvested for sequencing analysis at TRAC, TRBC1, TRBC2 and CIITA loci. NGS analysis was performed as described in Example 1. Table 24 and FIGS. 16A-16L show mean percent editing for these cells. For each guide, data from one representative donor is shown in Table 24.

TABLE 24
Mean percent editing.

| | sgRNA | C to T | | | C to A/G | | | Indels | | |
|---|---|---|---|---|---|---|---|---|---|---|
| gRNA | dose (μg/mL) | Mean | SD | n | Mean | SD | n | Mean | SD | n |
| G021469 | 0.0003 | 2.27 | 0.28 | 3 | 0.86 | 0.05 | 3 | 0.33 | 0.08 | 3 |
| (TRAC) | 0.0012 | 7.70 | 0.24 | 3 | 0.88 | 0.02 | 3 | 0.24 | 0.05 | 3 |
| | 0.0049 | 25.76 | 1.03 | 3 | 0.87 | 0.03 | 3 | 0.30 | 0.07 | 3 |
| | 0.0195 | 62.68 | 1.69 | 3 | 0.92 | 0.02 | 3 | 0.27 | 0.07 | 3 |
| | 0.0781 | 91.88 | 0.16 | 3 | 1.12 | 0.03 | 3 | 0.52 | 0.08 | 3 |
| | 0.3125 | 96.90 | 0.59 | 3 | 1.12 | 0.07 | 3 | 0.69 | 0.15 | 3 |
| | 1.25 | 97.42 | 0.39 | 3 | 1.24 | 0.02 | 3 | 0.88 | 0.19 | 3 |
| | 5 | 97.01 | 0.40 | 3 | 1.71 | 0.10 | 3 | 0.92 | 0.08 | 3 |
| G021481 | 0.00008 | 0.83 | 0.15 | 3 | 0.55 | 0.11 | 3 | 0.22 | 0.07 | 3 |
| (TRAC) | 0.00031 | 2.57 | 0.49 | 3 | 0.68 | 0.10 | 3 | 0.14 | 0.06 | 3 |
| | 0.00122 | 8.56 | 0.76 | 3 | 0.51 | 0.10 | 3 | 0.19 | 0.08 | 3 |
| | 0.00488 | 29.59 | 1.22 | 3 | 0.56 | 0.09 | 3 | 0.38 | 0.17 | 3 |
| | 0.01953 | 67.99 | 0.88 | 3 | 0.89 | 0.25 | 3 | 0.42 | 0.06 | 3 |
| | 0.07813 | 92.54 | 1.51 | 3 | 0.66 | 0.06 | 3 | 0.58 | 0.18 | 3 |
| | 0.3125 | 97.06 | 0.30 | 3 | 0.71 | 0.02 | 3 | 0.99 | 0.03 | 3 |
| | 1.25 | 96.85 | 0.44 | 3 | 0.93 | 0.07 | 3 | 1.71 | 0.10 | 3 |
| G028935 | 0.0003 | 10.56 | 0.30 | 3 | 0.83 | 0.06 | 3 | 0.12 | 0.01 | 3 |
| (TRAC) | 0.0012 | 33.70 | 1.30 | 3 | 1.05 | 0.06 | 3 | 10.19 | 0.04 | 3 |
| | 0.0049 | 72.47 | 1.20 | 3 | 1.16 | 0.13 | 3 | 0.33 | 0.07 | 3 |
| | 0.0195 | 94.40 | 0.52 | 3 | 1.43 | 0.20 | 3 | 0.42 | 0.10 | 3 |
| | 0.0781 | 96.53 | 0.68 | 3 | 1.78 | 0.25 | 3 | 0.52 | 0.09 | 3 |
| | 0.3125 | 96.50 | 0.86 | 3 | 2.02 | 0.24 | 3 | 0.74 | 0.28 | 3 |
| | 1.25 | 96.32 | 0.28 | 3 | 2.27 | 0.08 | 3 | 1.02 | 0.05 | 3 |
| | 5 | 95.22 | 0.44 | 3 | 2.84 | 0.13 | 3 | 1.49 | 0.12 | 3 |
| G028939 | 0.0003 | 2.63 | 0.49 | 2 | 1.37 | 0.41 | 2 | 10.20 | 0.02 | 2 |
| (TRAC) | 0.0012 | 9.65 | 0.16 | 2 | 1.21 | 0.90 | 2 | 0.13 | 0.02 | 2 |
| | 0.0049 | 29.60 | 1.03 | 2 | 1.42 | 0.69 | 2 | 0.19 | 0.00 | 1 |
| | 0.0195 | 66.33 | 2.98 | 2 | 1.68 | 0.47 | 2 | 0.33 | 0.04 | 2 |
| | 0.0781 | 192.37 | 0.69 | 2 | 1.93 | 0.34 | 2 | 0.60 | 0.30 | 2 |
| | 0.3125 | 94.59 | 0.32 | 2 | 1.65 | 0.91 | 2 | 0.39 | 0.30 | 2 |
| | 1.25 | n.d. | | | | | | | | |
| | 5 | 94.89 | 4.17 | 2 | 1.87 | 0.03 | 2 | 0.21 | 0.08 | 2 |
| G028943 | 0.0003 | 14.74 | 0.27 | 3 | 0.67 | 0.03 | 3 | 0.38 | 0.01 | 3 |
| (TRAC) | 0.0012 | 44.84 | 0.69 | 3 | 0.73 | 0.03 | 3 | 0.50 | 0.04 | 3 |
| | 0.0049 | 83.57 | 0.21 | 3 | 0.67 | 0.06 | 3 | 0.61 | 0.10 | 3 |
| | 0.0195 | 97.05 | 0.26 | 3 | 0.72 | 0.12 | 3 | 0.79 | 0.11 | 3 |
| | 0.0781 | 97.65 | 0.48 | 3 | 0.79 | 0.21 | 3 | 0.93 | 0.21 | 3 |
| | 0.3125 | 197.32 | 0.24 | 3 | 0.97 | 0.13 | 3 | 1.26 | 0.22 | 3 |
| | 1.25 | 96.51 | 0.35 | 3 | 1.37 | 0.09 | 3 | 1.82 | 0.34 | 3 |
| | 5 | 95.41 | 0.32 | 3 | 1.92 | 0.24 | 3 | 2.26 | 0.17 | 3 |
| G028986 | 0.0003 | 2.31 | 0.41 | 3 | 1.60 | 0.54 | 3 | 0.14 | 0.01 | 3 |
| (TRBC1) | 0.0012 | 8.20 | 0.37 | 3 | 1.48 | 0.16 | 3 | 10.15 | 0.03 | 3 |
| | 0.0049 | 26.27 | 1.69 | 3 | 1.69 | 0.31 | 3 | 0.26 | 0.08 | 3 |
| | 0.0195 | 62.21 | 0.95 | 3 | 1.73 | 0.13 | 3 | 0.52 | 0.22 | 3 |
| | 0.0781 | 92.08 | 0.61 | 3 | 1.33 | 0.09 | 3 | 10.65 | 0.20 | 3 |
| | 0.3125 | 96.37 | 0.34 | 3 | 1.48 | 0.06 | 3 | 1.12 | 0.46 | 3 |
| | 1.25 | 97.18 | 0.23 | 3 | 1.52 | 0.21 | 3 | 0.98 | 0.11 | 3 |
| | 5 | 96.58 | 0.35 | 3 | 1.77 | 0.14 | 3 | 1.30 | 0.32 | 3 |
| G029006 | 0.0003 | 3.00 | 0.73 | 3 | 0.55 | 0.02 | 3 | 0.25 | 0.03 | 3 |
| (TRBC2) | 0.0012 | 9.04 | 0.86 | 3 | 0.57 | 0.07 | 3 | 0.34 | 0.03 | 3 |
| | 0.0049 | 28.73 | 0.77 | 3 | 0.59 | 0.06 | 3 | 0.89 | 0.03 | 3 |
| | 0.0195 | 67.16 | 0.57 | 3 | 0.77 | 0.14 | 3 | 2.01 | 0.25 | 3 |
| | 0.0781 | 91.90 | 0.42 | 3 | 0.84 | 0.02 | 3 | 3.50 | 0.36 | 3 |
| | 0.3125 | 93.22 | 10.44 | 3 | 0.95 | 0.08 | 3 | 5.04 | 0.49 | 3 |
| | 1.25 | 91.67 | 10.70 | 3 | 1.42 | 0.13 | 3 | 6.27 | 0.41 | 3 |
| | 5 | 91.91 | 0.59 | 3 | 1.73 | 0.16 | 3 | 5.85 | 0.54 | 3 |
| G029007 | 0.0003 | 1.38 | 0.35 | 3 | 0.61 | 0.01 | 3 | 0.09 | 0.00 | 3 |
| (TRBC2) | 0.0012 | 3.96 | 0.46 | 3 | 0.69 | 0.05 | 3 | 0.11 | 0.01 | 3 |
| | 0.0049 | 12.87 | 0.75 | 3 | 0.68 | 0.13 | 3 | 0.16 | 0.03 | 3 |
| | 0.0195 | 38.66 | 10.91 | 3 | 0.95 | 0.32 | 3 | 0.25 | 0.01 | 3 |
| | 0.0781 | 79.87 | 0.93 | 3 | 0.74 | 0.07 | 3 | 0.43 | 0.08 | 3 |
| | 0.3125 | 96.82 | 0.18 | 3 | 0.75 | 0.03 | 3 | 0.62 | 0.06 | 3 |
| | 1.25 | 97.63 | 0.22 | 3 | 1.01 | 0.26 | 3 | 0.73 | 0.09 | 3 |
| | 5 | 97.44 | 0.25 | 3 | 10.93 | 0.12 | 3 | 0.86 | 0.08 | 3 |
| G026584 | 0.0003 | 1.30 | 0.06 | 3 | 0.66 | 0.03 | 3 | 0.08 | 0.03 | 3 |
| (CIITA) | 0.0012 | 3.97 | 0.40 | 3 | 0.60 | 0.04 | 3 | 0.10 | 0.01 | 3 |
| | 0.0049 | 13.87 | 0.18 | 3 | 0.61 | 0.07 | 3 | 0.10 | 0.04 | 3 |
| | 0.0195 | 42.35 | 0.82 | 3 | 0.59 | 0.04 | 3 | 0.17 | 0.04 | 3 |
| | 0.0781 | 82.44 | 0.99 | 3 | 0.64 | 0.05 | 3 | 0.29 | 0.08 | 3 |
| | 0.3125 | 97.03 | 0.60 | 3 | 0.77 | 0.32 | 3 | 0.43 | 0.08 | 3 |
| | 1.25 | 98.01 | 0.10 | 3 | 0.87 | 0.12 | 3 | 0.66 | 0.08 | 3 |
| | 5 | 97.83 | 0.23 | 3 | 0.97 | 0.03 | 3 | 0.68 | 0.06 | 3 |
| G029123 | 0.0003 | 0.68 | 0.10 | 3 | 10.27 | 0.04 | 3 | 0.12 | 0.01 | 3 |
| (CIITA) | 0.0012 | 2.19 | 0.28 | 3 | 0.31 | 0.06 | 3 | 0.15 | 0.08 | 3 |
| | 0.0049 | 7.60 | 0.52 | 3 | 0.31 | 0.05 | 3 | 0.17 | 0.02 | 3 |
| | 0.0195 | 27.13 | 1.24 | 3 | 0.32 | 0.11 | 3 | 0.15 | 0.04 | 3 |
| | 0.0781 | 65.16 | 0.17 | 3 | 10.27 | 0.02 | 3 | 0.18 | 0.05 | 3 |
| | 0.3125 | 94.17 | 0.27 | 3 | 0.30 | 0.05 | 3 | 0.18 | 0.05 | 3 |
| | 1.25 | 98.54 | 0.52 | 3 | 0.41 | 0.13 | 3 | 0.25 | 0.01 | 3 |
| | 5 | 98.37 | 0.22 | 3 | 0.46 | 0.07 | 3 | 0.17 | 0.04 | 3 |
| G029131 | 0.0003 | 2.43 | 0.08 | 3 | 0.25 | 0.03 | 3 | 0.15 | 0.03 | 3 |
| (CIITA) | 0.0012 | 7.66 | 0.42 | 3 | 0.24 | 0.03 | 3 | 0.19 | 0.07 | 3 |
| | 0.0049 | 24.67 | 0.25 | 3 | 0.34 | 0.08 | 3 | 0.29 | 0.08 | 3 |
| | 0.0195 | 60.40 | 0.99 | 3 | 0.32 | 0.07 | 3 | 0.58 | 0.04 | 3 |
| | 0.0781 | 91.11 | 0.39 | 3 | 0.46 | 0.04 | 3 | 0.95 | 0.08 | 3 |
| | 0.3125 | 97.56 | 0.12 | 3 | 0.42 | 0.10 | 3 | 1.27 | 0.06 | 3 |
| | 1.25 | 97.70 | 0.10 | 3 | 0.52 | 0.01 | 3 | 1.53 | 0.09 | 3 |
| | 5 | 97.59 | 0.29 | 3 | 0.71 | 0.02 | 3 | 1.46 | 0.35 | 3 |
| | 0.0003 | 0.46 | 0.07 | 3 | 0.39 | 0.02 | 3 | 0.15 | 0.03 | 3 |
| | 0.0012 | 1.42 | 0.25 | 3 | 0.39 | 0.03 | 3 | 0.14 | 0.07 | 3 |
| | 0.0049 | 5.11 | 0.21 | 3 | 0.43 | 0.03 | 3 | 0.12 | 0.01 | 3 |
| G029172 | 0.0195 | 17.07 | 0.41 | 3 | 0.59 | 0.21 | 3 | 0.22 | 0.04 | 3 |
| (CIITA) | 0.0781 | 46.68 | 1.19 | 3 | 0.45 | 0.09 | 3 | 0.43 | 0.12 | 3 |
| | 0.3125 | 82.18 | 0.17 | 3 | 0.54 | 0.07 | 3 | 0.52 | 0.09 | 3 |
| | 1.25 | 95.09 | 0.61 | 3 | 0.55 | 0.10 | 3 | 0.54 | 0.08 | 3 |
| | 5 | 94.17 | 0.07 | 3 | 0.89 | 0.30 | 3 | 0.62 | 0.20 | 3 |

“n.d.” indicates no data.

### 16.4 Flow Cytometry

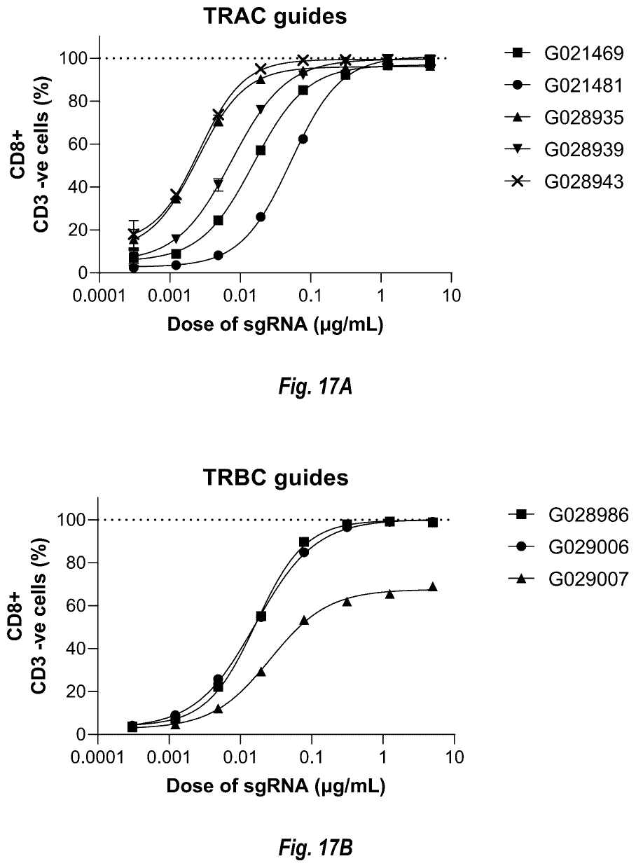

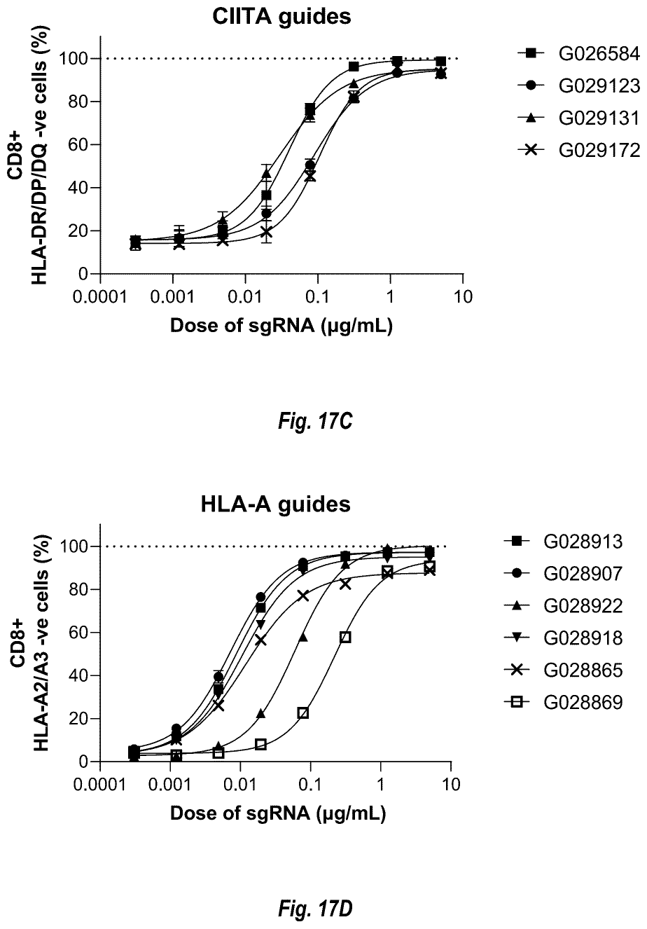

On Day 10, cells were harvested for analysis by flow cytometry. For flow cytometric analysis, cells were washed in FACS buffer (PBS+2% FBS+2 mM EDTA). Cells were incubated in a cocktail of antibodies targeting CD3 (Biolegend 317336), CD4 (Biolegend 300538), CD8 (Biolegend 301051), HLA-A2 (Biolegend 343320), HLA-A3 (Invitrogen, 17-5754-42), HLA-B7 (Miltenyi Biotec, 130-120-234), HLA-DR, DP, DQ (Biolegend 361708), and ViaKrome 808 Fixable Viability Dye (Beckman Coulter, C36628). T cells were subsequently washed and analyzed on a Cytoflex instrument (Beckman Coulter). Data analysis was performed using FlowJo software package (v.10.6.1). T cells were gated based on size, viability, CD8 positivity, and expression of markers indicated in Table 25. Flow cytometry data for CD8+ T cells are shown in Table 25 and FIGS. 17A-17D. CD3-, HLA-A2-/HLA-A3-, and HLA-DR, DP, DQ-cell populations indicate efficient disruption of TRAC, TRBC1 or TRBC2; HLA-A; or CIITA, respectively. Data from one representative donor is shown in Table 25. Similar results were seen in both donors.

TABLE 25
Mean percent CD8+ T cells expressing surface markers in representative
donor. CIITA guides are reported as mean percent HLA-DP, DQ, DR-
cells. HLA-A guides are reported as mean percent HLA-A2/A3- cells.
TRAC and TRBC guides are reported as mean percent CD3− cells.

| | sgRNA | | | | | sgRNA | | | |
|---|---|---|---|---|---|---|---|---|---|
| | dose | | | | | dose | | | |
| Guide | (ug/ml) | Mean | SD | n | Guide | (ug/ml) | Mean | SD | n |
| G026584 | 0.0003 | 14.8 | 2.5 | 3 | G029123 | 0.0003 | 14.3 | 3.4 | 3 |
| CIITA | 0.0012 | 16.3 | 3.7 | 3 | CIITA | 0.0012 | 16.6 | 3.9 | 3 |
| | 0.0049 | 20.5 | 4.2 | 3 | | 0.0049 | 18.4 | 3.6 | 3 |
| | 0.0195 | 36.5 | 6.5 | 2 | | 0.0195 | 28.1 | 3.4 | 3 |
| | 0.0781 | 76.5 | 2.6 | 3 | | 0.0781 | 50.6 | 2.7 | 3 |
| | 0.3125 | 96.3 | 0.2 | 3 | | 0.3125 | 81.4 | 1.9 | 3 |
| | 1.25 | 98.8 | 0.3 | 3 | | 1.25 | 93.2 | 1.1 | 3 |
| | 5 | 98.7 | 0.2 | 3 | | 5 | 92.6 | 0.3 | 3 |
| G029131 | 0.0003 | 16.2 | 1.7 | 3 | G029172 | 0.0003 | 13.9 | 0.7 | 3 |
| CIITA | 0.0012 | 17.5 | 5.0 | 3 | CIITA | 0.0012 | 13.8 | 1.0 | 3 |
| | 0.0049 | 25.2 | 3.6 | 3 | | 0.0049 | 15.5 | 1.4 | 3 |
| | 0.0195 | 46.9 | 4.0 | 3 | | 0.0195 | 19.5 | 5.2 | 3 |
| | 0.0781 | 73.7 | 3.3 | 3 | | 0.0781 | 45.4 | 2.3 | 3 |
| | 0.3125 | 88.5 | 1.3 | 3 | | 0.3125 | 82.1 | 2.8 | 3 |
| | 1.25 | 94.6 | 0.7 | 3 | | 1.25 | 95.8 | 0.4 | 3 |
| | 5 | 94.4 | 1.5 | 3 | | 5 | 93.1 | 1.5 | 3 |
| G028922 | 0.0003 | 2.6 | 0.4 | 3 | G028865 | 0.0003 | 4.7 | 0.2 | 3 |
| HLA-A | 0.0012 | 2.7 | 0.9 | 3 | HLA-A | 0.0012 | 10.2 | 0.5 | 3 |
| | 0.0049 | 7.4 | 0.7 | 3 | | 0.0049 | 26.1 | 1.1 | 3 |
| | 0.0195 | 22.6 | 0.8 | 3 | | 0.0195 | 56.6 | 1.7 | 3 |
| | 0.0781 | 58.1 | 1.0 | 3 | | 0.0781 | 77.1 | 1.7 | 3 |
| | 0.3125 | 91.9 | 0.9 | 3 | | 0.3125 | 82.5 | 1.1 | 3 |
| | 1.25 | 99.1 | 0.1 | 3 | | 1.25 | 87.5 | 1.1 | 3 |
| | 5 | 98.3 | 0.6 | 3 | | 5 | 89.0 | 0.8 | 3 |
| G028869 | 0.0003 | 2.4 | 0.8 | 3 | G028907 | 0.0003 | 5.8 | 0.3 | 3 |
| HLA-A | 0.0012 | 2.1 | 1.3 | 3 | HLA-A | 0.0012 | 15.4 | 0.2 | 3 |
| | 0.0049 | 2.7 | 0.2 | 3 | | 0.0049 | 39.5 | 2.9 | 3 |
| | 0.0195 | 7.1 | 0.5 | 3 | | 0.0195 | 76.5 | 0.5 | 3 |
| | 0.0781 | 19.7 | 2.3 | 3 | | 0.0781 | 92.4 | 0.4 | 3 |
| | 0.3125 | 47.9 | 1.4 | 3 | | 0.3125 | 95.7 | 0.7 | 3 |
| | 1.25 | 68.7 | 0.6 | 3 | | 1.25 | 97.7 | 0.5 | 3 |
| | 5 | 66.9 | 0.6 | 3 | | 5 | 97.0 | 0.2 | 3 |
| G028913 | 0.0003 | 4.3 | 0.7 | 3 | G028918 | 0.0003 | 4.8 | 0.4 | 3 |
| HLA-A | 0.0012 | 11.8 | 0.7 | 3 | HLA-A | 0.0012 | 10.0 | 1.2 | 3 |
| | 0.0049 | 33.7 | 0.5 | 3 | | 0.0049 | 31.0 | 1.0 | 3 |
| | 0.0195 | 71.5 | 0.9 | 3 | | 0.0195 | 63.4 | 0.2 | 3 |
| | 0.0781 | 90.8 | 0.9 | 3 | | 0.0781 | 88.4 | 1.0 | 3 |
| | 0.3125 | 95.4 | 0.5 | 3 | | 0.3125 | 93.5 | 0.4 | 3 |
| | 1.25 | 97.3 | 0.6 | 3 | | 1.25 | 94.3 | 0.5 | 3 |
| | 5 | 97.4 | 0.3 | 3 | | 5 | 94.5 | 0.1 | 3 |
| G029006 | 0.0003 | 4.2 | 0.8 | 3 | G028986 | 0.0003 | 3.8 | 1.2 | 3 |
| TRBC | 0.0012 | 9.0 | 1.3 | 3 | TRBC | 0.0012 | 7.3 | 0.2 | 3 |
| | 0.0049 | 25.8 | 0.9 | 3 | | 0.0049 | 22.2 | 0.3 | 3 |
| | 0.0195 | 54.7 | 1.5 | 3 | | 0.0195 | 55.1 | 1.1 | 3 |
| | 0.0781 | 84.8 | 0.7 | 3 | | 0.0781 | 89.7 | 1.3 | 3 |
| | 0.3125 | 96.6 | 0.2 | 3 | | 0.3125 | 97.8 | 0.2 | 3 |
| | 1.25 | 99.1 | 0.2 | 3 | | 1.25 | 99.1 | 0.1 | 3 |
| | 5 | 99.0 | 0.2 | 3 | | 5 | 98.8 | 0.1 | 3 |
| G029007 | 0.0003 | 3.3 | 0.4 | 3 | G028943 | 0.0003 | 17.97 | 6.36 | 3 |
| TRBC | 0.0012 | 4.7 | 0.7 | 3 | TRAC | 0.0012 | 36.57 | 0.49 | 3 |
| | 0.0049 | 12.1 | 1.5 | 3 | | 0.0049 | 73.73 | 1.19 | 3 |
| | 0.0195 | 29.4 | 1.7 | 3 | | 0.0195 | 95.03 | 0.32 | 3 |
| | 0.0781 | 53.4 | 2.0 | 3 | | 0.0781 | 99.00 | 0.44 | 3 |
| | 0.3125 | 61.9 | 0.8 | 3 | | 0.3125 | 99.27 | 0.06 | 3 |
| | 1.25 | 65.5 | 1.1 | 3 | | 1.25 | 99.33 | 0.25 | 3 |
| | 5 | 69.0 | 1.4 | 3 | | 5 | 98.97 | 0.42 | 3 |
| G021469 | 0.0003 | 7.04 | 3.60 | 3 | G021481 | 0.00008 | 2.43 | 0.47 | 3 |
| TRAC | 0.0012 | 8.84 | 0.27 | 3 | TRAC | 0.00031 | 3.58 | 0.77 | 3 |
| | 0.0049 | 24.50 | 1.04 | 3 | | 0.00122 | 8.21 | 1.05 | 3 |
| | 0.0195 | 57.13 | 1.20 | 3 | | 0.00488 | 26.03 | 0.35 | 3 |
| | 0.0781 | 85.10 | 1.35 | 3 | | 0.01953 | 62.40 | 2.12 | 3 |
| | 0.3125 | 92.30 | 2.12 | 3 | | 0.07813 | 92.70 | 0.70 | 3 |
| | 1.25 | 96.90 | 0.26 | 3 | | 0.3125 | 99.00 | 0.10 | 3 |
| | 5 | 97.57 | 0.40 | 3 | | 1.25 | 99.47 | 0.21 | 3 |
| G028935 | 0.0003 | 15.60 | 4.65 | 3 | G028939 | 0.0003 | 7.87 | 3.58 | 3 |
| TRAC | 0.0012 | 34.63 | 0.83 | 3 | TRAC | 0.0012 | 15.70 | 0.10 | 3 |
| | 0.0049 | 70.97 | 2.47 | 3 | | 0.0049 | 40.90 | 2.88 | 3 |
| | 0.0195 | 90.27 | 0.35 | 3 | | 0.0195 | 75.90 | 1.28 | 3 |
| | 0.0781 | 94.17 | 1.00 | 3 | | 0.0781 | 92.10 | 0.66 | 3 |
| | 0.3125 | 95.57 | 0.76 | 3 | | 0.3125 | 98.43 | 0.06 | 3 |
| | 1.25 | 96.63 | 0.25 | 3 | | 1.25 | 99.70 | 0.10 | 3 |
| | 5 | 96.47 | 0.06 | 3 | | 5 | 99.53 | 0.06 | 3 |

### Example 17. Off-Target Analysis

### 17.1 Biochemical Off-Target Analysis

A biochemical method (See, e.g., Cameron et al., Nature Methods. 6, 600-606; 2017) was used to determine potential off-target genomic sites cleaved by Nme2Cas9 using specific guides targeting HLA-A, TRAC, TRBC1 & TRBC2, and CIITA, respectively. Guide RNAs shown in Table 26 were screened using NA24385 genomic DNA (Coriell Institute) alongside control guides. The number of on target and potential off-target cleavage sites were detected using a guide concentration of 192 nM gRNA and 64 nM Nme2Cas9 protein in the biochemical assay for which results are shown in Table 26.

Digenome sequencing was also performed by methods known in the art for discovery of potential off-target sites for select guides using Nme2 base editor. Data not shown.

TABLE 26
Biochemical Off-Target Analysis

| Guide ID | Target Gene | Sites |
|---|---|---|
| G028865 | HLA-A | 18 |
| G028869 | HLA-A | 11 |
| G028907 | HLA-A | 7 |
| G028913 | HLA-A | 13 |
| G028918 | HLA-A | 4 |
| G028922 | HLA-A | 14 |
| G028935 | TRAC | 4 |
| G028939 | TRAC | 4 |
| G028943 | TRAC | 2 |
| G028986 | TRBC1 | 8 |
| G029006 | TRBC2 | 9 |
| G029007 | TRBC2 | 7 |
| G026584 | CIITA | 6 |
| G029123 | CIITA | 1 |
| G029131 | CIITA | 2 |
| G029172 | CIITA | 4 |
| G021557 | VEGFA | 5 |
| G021558 | VEGFA | 1 |
| G021567 | VEGFA | 2 |

### 17.2 Targeted Sequencing at Potential Off-Target Sites

A two-phase process was applied to identify and then confirm potential off-target editing. In phase one, a computational off-target prediction method Cas-OFFinder (Bae et al., 2014) was combined with the biochemical off-target discovery assay described above to identify potential off-target editing sites. The final phase confirms editing at the potential off-target loci through the detection of C-to-T mutations using NGS in genome-edited cells. A multiplex PCR based rhAMPSeq (RNase H2 dependent PCR Amplification for Next Generation Sequencing) assay or NGS as described in Example 1 was employed to characterize potential off-target editing in cells.

Samples were prepared in triplicate for two T cell donors. T cells were prepared as described in Example 1. Cells were treated simultaneously with 3 LNPs, each formulated with a single RNA cargo of Nme2BC22 mRNA, UGI mRNA, or a select sgRNA as listed in Table 27. LNPs were generally prepared as described in Example 1 with lipid molar ratio of 35 Lipid A:47.5 cholesterol:15 DSPC:2.5 PEG. LNPs were pre-incubated in 10 μg/mL of human ApoE3. Approximately 50,000 T cells were treated with LNPs measured by RNA weight as follows: 100 ng of Nme2BC22 mRNA, 50 ng of UGI mRNA, and sgRNA at the dose shown in Table 27. Cells were incubated at 37° C. for about 24 hours then resuspended in fresh media for further growth. Approximately 96 hours (4 days) after LNP treatment, cells were harvested and NGS analysis was performed generally as described in Example 1 or via rhAmpSeq CRISPR Analysis System (IDT) by the manufacturer's protocol using cell lysate. NGS analysis used primers designed to identify percent C-to-T mutations at predicted off-target sites. Results of the potential off target site sequencing analysis from two donors are summarized in Table 27. Potential off target sites were considered to have confirmed base editing when the mean % C to T editing was statistically significant (P value of 0.05 or less) compared to donor-matched untreated controls. Edited sequence reads were manually inspected for potential off target sites reaching these criteria to confirm edit-relevant C to T repair structures.

TABLE 27
Editing confirmation at potential off target sites by sequencing

| | | | | Loci with |
|---|---|---|---|---|
| | | | Loci | confirmed |
| Guide | Target | LNP Dose (ng) | characterized | base editing |
| G028907 | HLA-A | 23.8 | 7 | 3 |
| G028913 | HLA-A | 31.7 | 6 | 2 |
| G028918 | HLA-A | 28.6 | 4 | 3 |
| G028939 | TRAC | 21.5 | 3 | 0 |
| G028943 | TRAC | 5.8 | 1 | 0 |
| G028986 | TRBC | 37.7 | 7 | 1 |
| G029006 | TRBC | 55.0 | 7 | 3 |
| G026584 | CIITA | 70.6 | 7 | 2 |
| G029131 | CIITA | 96.8 | 1 | 0 |

1. An engineered cell, which has reduced or eliminated surface expression of HLA-A relative to an unmodified cell, comprising a genetic modification in the HLA-A gene, wherein the genetic modification comprises at least one nucleotide within the genomic coordinates chosen from: a) chr6:29942540-29945459: b) chr6:29942891-29942915: chr6:29942609-29942633: chr6:29944266-29944290: chr6:29942889-29942913: chr6:29944471-29944495; and chr6:29944470-29944494; and c) chr6:29942785-29942809.

2. The engineered cell of claim 1, wherein the genetic modification comprises:
a) at least one nucleotide within the genomic coordinates chosen from the genomic coordinates listed in Table 1;
b) at least one nucleotide within the genomic coordinates targeted by a guide RNA comprising a guide sequence of any one of SEQ ID NOs: 66, 61, 2-60, 62-65, 67-80; and/or
c) an indel, a C to T substitution, or an A to G substitution within the genomic coordinates.

3. (canceled)

4. (canceled)

5. (canceled)

6. (canceled)

7. (canceled)

8. (canceled)

9. The engineered cell of claim 1, wherein the cell is: a) homozygous for HLA-C or b) homozygous for HLA-B and homozygous for HLA-C.

10. (canceled)

11. A composition comprising an HLA-A guide RNA, wherein the HLA-A guide RNA comprises
i) a guide sequence chosen from SEQ ID NOs: 66, 61, 2-60, 62-65, and 67-80;
ii) at least 19, 20, 21, 22, 23, or 24 contiguous nucleotides of a sequence chosen from SEQ ID NOs: 66, 61, 2-60, 62-65, and 67-80; or
iii) a guide sequence that is at least 95%, 90%, or 85% identical to a sequence chosen from SEQ ID NOs: 66, 61, 2-60, 62-65, and 67-80;
iv) a sequence that comprises 10 contiguous nucleotides±10 nucleotides of a genomic coordinate listed in Table 1;
v) at least 20, 21, 22, 23, or 24, contiguous nucleotides of a sequence chosen from iv); or
vi) a guide sequence that is at least 95%, 90%, or 85% identical to a sequence chosen from iv).

12. A method of a) making an engineered human cell, which has reduced or eliminated surface expression of HLA-A protein relative to an unmodified cell, or b) reducing surface expression of HLA-A protein in a human cell relative to an unmodified cell, the method comprising contacting a cell with a composition comprising an HLA-A guide RNA, wherein the HLA-A guide RNA comprises
i) a guide sequence chosen from SEQ ID NOs: 66, 61, 2-60, 62-65, and 67-80;
ii) at least 20, 21, 22, 23, or 24 contiguous nucleotides of a sequence chosen from SEQ ID NOs: 66, 61, 2-60, 62-65, and 67-80; or
iii) a guide sequence that is at least 95%, 90%, or 85% identical to a sequence chosen from SEQ ID NOs: 66, 61, 2-60, 62-65, and 67-80;
iv) a sequence that comprises 10 contiguous nucleotides±10 nucleotides of a genomic coordinate listed in Table 1;
v) at least 20, 21, 22, 23, or 24 contiguous nucleotides of a sequence chosen from iv); or
vi) a guide sequence that is at least 95%, 90%, or 85% identical to a sequence chosen from iv).

13. (canceled)

14. The composition of claim 11, wherein the HLA-A guide RNA comprises:
a) a guide sequence chosen from SEQ ID NOs: 66, 61, 13, 55, 70, and 71,
b) a guide sequence chosen from SEQ ID NOs: 61, 66, 13, 17, 55, and 70;
c) a guide sequence comprising the sequence of SEQ ID NO: 61; or
d) a guide sequence comprising the sequence of SEQ ID NO: 66.

15. (canceled)

16. (canceled)

17. (canceled)

18. A population of cells comprising the engineered cells of claim 1.

19. A pharmaceutical composition comprising the engineered cell of claim 1.

20. An engineered human cell, which has reduced or eliminated surface expression of TRAC relative to an unmodified cell, comprising a genetic modification in the TRAC gene, wherein the genetic modification comprises: a) at least one nucleotide within the genomic coordinates chr14:22547505-22551621 or chr14:22547462-22551621; or b) an indel, a C to T substitution, or an A to G substitution within the genomic coordinates chosen from: chr14:22550574-22550598: chr14:22550544-22550568: chr14:22547505-22547529; chr14:22547525-22547549; and chr14:22547674-22547698.

21. The engineered cell of claim 20, wherein the genetic modification comprises: a) at least one nucleotide within the genomic coordinates chosen from the genomic coordinates listed in Table 2 or at least one nucleotide within the genomic coordinates targeted by a guide RNA comprising a guide sequence chosen from SEQ ID NOs: 111, 107, 101-106, 108-110, and 112-120; and/or
b) at least one nucleotide within the genomic coordinates chosen from: chr14:22550574-22550598: chr14:22550544-22550568: chr14:22547505-22547529; chr14:22547525-22547549; and chr14:22547674-22547698.

22. (canceled)

23. (canceled)

24. (canceled)

25. A composition comprising:
a) a TRAC guide RNA comprising a guide sequence that i) targets a TRAC genomic target sequence; or ii) directs an RNA-guided DNA binding agent to induce a double stranded break (DSB) or a single-stranded break (SSB) in a TRAC genomic target sequence comprising at least 10 contiguous nucleotides within the genomic coordinates chosen from: chr14:22550574-22550598; chr14:22550544-22550568; chr14:22547505-22547529; chr14:22547525-22547549; and chr14:22547674-22547698; or
b) a TRAC guide RNA, wherein the TRAC guide RNA comprises:
i) a guide sequence chosen from SEQ ID NOs: 111, 107, 101-106, 108-110, and 112-120;
ii) at least 20, 21, 22, 23, or 24 contiguous nucleotides of a sequence chosen from SEQ ID NOs: 111, 107, 101-106, 108-110, and 112-120; or
iii) a guide sequence that is at least 95%, 90%, or 85% identical to a sequence chosen from SEQ ID NOs: 111, 107, 101-106, 108-110, and 112-120;
iv) a sequence that comprises 10 contiguous nucleotides±10 nucleotides of a genomic coordinate listed in Table 2;
v) at least 20, 21, 22, 23, or 24 contiguous nucleotides of a sequence chosen from iv); or
vi) a guide sequence that is at least 95%, 90%, or 85% identical to a sequence chosen from iv).

26. (canceled)

27. (canceled)

28. (canceled)

29. A method of I) making an engineered human cell, which has reduced or eliminated surface expression of TRAC protein relative to an unmodified cell, or II) reducing surface expression of TRAC protein in a human cell relative to an unmodified cell, the method comprising contacting a cell with a composition comprising a TRAC guide RNA, wherein the TRAC guide RNA comprises:
i) a guide sequence chosen from SEQ ID NOs: 111, 107, 101-106, 108-110, and 112-120;
ii) at least 20, 21, 22, 23, or 24 contiguous nucleotides of a sequence chosen from SEQ ID NOs: 111, 107, 101-106, 108-110, and 112-120; or
iii) a guide sequence that is at least 95%, 90%, or 85% identical to a sequence chosen from SEQ ID NOs: 111, 107, 101-106, 108-110, and 112-120; or
iv) a sequence that comprises 10 contiguous nucleotides±10 nucleotides of a genomic coordinate listed in Table 2;
v) at least 20, 21, 22, 23, or 24 contiguous nucleotides of a sequence chosen from iv); or
vi) a guide sequence that is at least 95%, 90%, or 85% identical to a sequence chosen from iv).

30. (canceled)

31. The composition of claim 25,
wherein the TRAC guide RNA comprises:
a) a guide sequence chosen from SEQ ID NOs: 111, 107, 101, 102, and 103;
b) a guide sequence comprising the sequence of SEQ ID NO: 107; or
c) a guide sequence comprising the sequence of SEQ ID NO: 111.

32. (canceled)

33. (canceled)

34. A pharmaceutical composition comprising the engineered cell of claim 20.

35. An engineered cell, which has reduced or eliminated surface expression of TRBC relative to an unmodified cell, comprising a genetic modification in the TRBC gene, wherein the genetic modification comprises:
I) at least one nucleotide within the genomic coordinates (a)_chr7:142791756-142802543; (b) chr7:142791862-142793149; (c) chr7: 142791756-142792721; or (d) chr7:142801104-142802543; or wherein the genetic modification comprises at least one nucleotide within the genomic coordinates targeted by a guide RNA comprising a guide sequence chosen from SEQ ID NOs: 215, 201-214, and 216-265; or
II) an indel, a C to T substitution, or an A to G substitution within the genomic coordinates chosen from: (a) chr7:142791862-142793149; (b) chr7:142791756-142792721; (c) chr7:142801104-142802543; (d) chr7:142792690-142792714 and chr7:142792693-142792717; (e) chr7:142791756-142791780; chr7:142791761-142791785; chr7:142791820-142791844; chr7:142791939-142791963; chr7:142791940-142791964; and chr7:142792004-142792028; (f) chr7:142801104-142801124; chr7:142802103-142802127; and chr7:142802106-142802130; and (g) chr7:142802103-142802127.

36. (canceled)

37. The engineered cell of claim 35, wherein the genetic modification comprises at least one nucleotide within the genomic coordinates chosen from:
a) chr7:142792690-142792714; and chr7:142792693-142792717;
b) chr7:142791756-142791780; chr7:142791761-142791785; chr7:142791820-142791844; chr7:142791939-142791963; chr7:142791940-142791964; and chr7:142792004-142792028;
c) chr7:142801104-142801124; chr7:142802103-142802127; and chr7:142802106-142802130; and
d) chr7:142802103-142802127; and chr7:142802106-14280213; and
e) the genomic coordinates listed in Table 3.

38. (canceled)

39. (canceled)

40. (canceled)

41. (canceled)

42. (canceled)

43. (canceled)

44. (canceled)

45. A composition comprising (a) a TRBC guide RNA, wherein the TRBC guide RNA comprises:
i) a guide sequence chosen from SEQ ID NOs: 215, 201-214, and 216-265;
ii) at least 20, 21, 22, 23, or 24, contiguous nucleotides of a sequence chosen from SEQ ID NOs: 215, 201-214, and 216-265;
iii) a guide sequence that is at least 95%, 90%, or 85% identical to a sequence chosen from SEQ ID NOs: 215, 201-214, and 216-265;
iv) a sequence that comprises 10 contiguous nucleotides±10 nucleotides of a genomic coordinate listed in Table 3;
v) at least 20, 21, 22, 23, or 24 contiguous nucleotides of a sequence chosen from iv); or
vi) a guide sequence that is at least 95%, 90%, or 85% identical to a sequence chosen from iv).

46. (canceled)

47. (canceled)

48. A method of I) making an engineered human cell, which has reduced or eliminated surface expression of TRBC protein relative to an unmodified cell, or II) reducing surface expression of TRBC protein in a human cell relative to an unmodified cell, the method comprising contacting a cell with TRBC guide RNA and, wherein the TRBC guide RNA comprises:
i) a guide sequence chosen from SEQ ID NOs: 215, 201-214, and 216-265;
ii) at least 20, 21, 22, 23, or 24 contiguous nucleotides of a sequence chosen from SEQ ID NOs: 215, 201-214, and 216-265;
iii) a guide sequence that is at least 95%, 90%, or 85% identical to a sequence chosen from SEQ ID NOs: 215, 201-214, and 216-265;
iv) a sequence that comprises 10 contiguous nucleotides±10 nucleotides of a genomic coordinate listed in Table 3;
v) at least 20, 21, 22, 23, or 24, contiguous nucleotides of a sequence chosen from iv); or
vi) a guide sequence that is at least 95%, 90%, or 85% identical to a sequence chosen from iv).

49. (canceled)

50. The composition of claim 45, wherein the TRBC guide RNA comprises a guide sequence of:
a) any one of SEQ ID NOs: 215, 216, 223, 224, 229, 230, 246, 259, and 260,
b) any one of SEQ ID NOs: 215, 216, 224, 229, 246, 259, and 260; or
c) any one of SEQ ID NOs: 215, 259, and 260.

51. (canceled)

52. (canceled)

53. A pharmaceutical composition comprising the engineered cell of claim 35.

54. An engineered cell, which has reduced or eliminated surface expression of MHC class II relative to an unmodified cell, comprising:
I) a genetic modification in the CIITA gene, wherein the genetic modification comprises at least one nucleotide within the genomic coordinates chosen from: (a) chr16:10877363-10907788; and (b) chr16:10906515-10908136;
II) a genetic modification in the CIITA gene, wherein the genetic modification comprises an indel, a C to T substitution, or an A to G substitution within the genomic coordinates chosen from: (a) chr16:10906643-10906667: chr16:10907504-10907528: chr16:10907508-10907532: chr16:10907539-10907559; chr16:10895658-10895682; chr16:10895668-10895692; chr16:10895750-10895774; chr16:10895753-10895777; chr16:10895754-10895778; chr16:10898684-10898708; chr16:10901529-10901553; chr16:10902121-10902145; chr16:10902701-10902725; chr16:10904726-10904750; chr16:10904760-10904784; chr16:10906493-10906517; chr16:10906515-10906539; chr16:10906631-10906655; chr16:10906636-10906660; chr16:10906770-10906794; chr16:10906788-10906812; chr16:10906789-10906813; chr16:10906816-10906840; chr16:10907148-10907172; chr16:10907254-10907278; chr16:10907331-10907355; chr16:10907477-10907501; chr16:10907497-10907521; chr16:10907503-10907527; and chr16:10907574-10907598; and (b) chr16:10907504-10907528; or
III) a genetic modification in the CIITA gene, wherein the genetic modification comprises an indel, a C to T substitution, or an A to G substitution within the genomic coordinates chosen from: a) chr16:10906643-10906667: chr16:10907504-10907528: chr16:10895658-10895682: chr16:10902701-10902725; chr16:10906493-10906517; chr16:10906631-10906655; chr16:10907477-10907501; chr16:10907497-10907521; chr16:10907504-10907528; and chr16:10907508-10907532; and
b) chr16:10906889-10906913.

55. (canceled)

56. (canceled)

57. (canceled)

58. (canceled)

59. (canceled)

60. (canceled)

61. (canceled)

62. A composition comprising a CIITA guide RNA, wherein the CIITA guide RNA comprises:
i) a guide sequence chosen from SEQ ID NOs: 422, 301-421, and 423-576; or
ii) at least 20, 21, 22, 23, or 24 contiguous nucleotides of a sequence chosen from SEQ ID NOs: 422, 301-421, and 423-576; or
iii) a guide sequence that is at least 95%, 90%, or 85% identical to a sequence chosen from SEQ ID NOs: 422, 301-421, and 423-576; or
iv) a sequence that comprises 10 contiguous nucleotides±10 nucleotides of a genomic coordinate listed in Table 4; or
v) at least 20, 21, 22, 23, or 24 contiguous nucleotides of a sequence chosen from iv); or
vi) a guide sequence that is at least 95%, 90%, or 85% identical to a sequence chosen from iv).

63. (canceled)

64. (canceled)

65. A method of I) making an engineered human cell, which has reduced or eliminated surface expression of MHC class II protein relative to an unmodified cell, or II) reducing surface expression of MHC class II protein in a human cell relative to an unmodified cell, the method comprising contacting a cell with a CIITA guide RNA, wherein the CIITA guide RNA comprises:
i) a guide sequence chosen from SEQ ID NOs: 422, 301-421, and 423-576; or
ii) at least 20, 21, 22, 23, or 24 contiguous nucleotides of a sequence chosen from SEQ ID NOs: 422, 301-421, and 423-576; or
iii) a guide sequence that is at least 95%, 90%, or 85% identical to a sequence chosen from SEQ ID NOs: 422, 301-421, and 423-576; or
iv) a sequence that comprises 10 contiguous nucleotides±10 nucleotides of a genomic coordinate listed in Table 4; or
v) at least 20, 21, 22, 23, or 24 contiguous nucleotides of a sequence chosen from iv); or
vi) a guide sequence that is at least 95%, 90%, or 85% identical to a sequence chosen from iv).

66. (canceled)

67. The composition of claim 62, wherein the CIITA guide RNA comprises a guide sequence of chosen from SEQ ID NOs: 422, 301, 302, 320, 321, 324, 326, 327, 332, 354, 361, 372, 400, 408, 414, 415, 419, 420, 428, 431, 432, 434, 451, 455, 458, 462, 463, 464, and 468.

68. (canceled)

69. A pharmaceutical composition comprising the engineered cell of claim 54.

70. (canceled)

71. (canceled)

72. (canceled)

73. (canceled)

74. (canceled)

75. (canceled)

76. The engineered cell of claim 54,
wherein the cell:
a) has reduced expression of TRAC protein on the surface of the cell;
b) has reduced expression of TRBC protein on the surface of the cell;
c) is an immune cell;
d) is a stem cell;
e) is a primary cell; or
f) is engineered with a genomic editing system.

77. (canceled)

78. (canceled)

79. (canceled)

80. (canceled)

81. (canceled)

82. (canceled)

83. (canceled)

84. (canceled)

85. (canceled)

86. (canceled)

87. (canceled)

88. (canceled)

89. (canceled)

90. (canceled)

91. (canceled)

92. (canceled)

93. (canceled)

94. (canceled)

95. (canceled)

96. (canceled)

97. (canceled)

98. (canceled)

99. (canceled)

100. (canceled)

101. (canceled)

102. (canceled)

103. (canceled)

104. A method of treating a disease or disorder comprising administering the engineered cell, of claim 54 to a subject in need thereof.

105. (canceled)

106. (canceled)

107. (canceled)

108. (canceled)
